SEQUENCE LISTING (3 sequences)

SEQ ID NO: 1 - DNA, 20 bp, Artificial Sequence
  Forward Primer
gtgtctgcggcgttttatca

SEQ ID NO: 2 - DNA, 21 bp, Artificial Sequence
  Reverse Primer
gacaaacgggcaacatacctt

SEQ ID NO: 3 - DNA, 28 bp, Artificial Sequence
  Taqman Probe
cctctkcatcctgctgctatgcctcatc

(19) United States
(12) Patent Application Publication
(10) Pub. No.: US 2024/0279227 A1
(43) Pub. Date: Aug. 22, 2024

(54) FUSED HETEROCYCLIC DERIVATIVES

(71) Applicant: Janssen Sciences Ireland Unlimited Company, Co Cork (IE)

(72) Inventors: Chunliang LU, Shanghai (CN); Gang DENG, Shanghai (CN); Lianzhu LIU, Shanghai (CN); Xiaoyu LI, Shanghai (CN); Zhiguo LIU, Shanghai (CN); Bingqing TANG, Anhui Province (CN); Wei CAI, Shanghai (CN); Shane PLUNKETT, Warminster, PA (US); Philip James PYE, Bridgewater, NJ (US); Lindsey Graham DERATT, North Wales, PA (US); Pierre Jean-Marie Bernard RABOISSON, Wavre (BE); Edgar JACOBY, Vosselaar (BE); Sandrine Céline GROSSE, Turnhout (BE); Zhanling CHENG, Shanghai (CN); Koen VANDYCK, Paal-Beringen (BE); Tim Hugo Maria JONCKERS, Heist-op-den-Berg (BE); Scott D. KUDUK, Harleysville, PA (US)

(21) Appl. No.: 18/565,664

(22) PCT Filed: Jun. 1, 2022
(86) PCT No.: PCT/CN2022/096525

§ 371 Date: Nov. 30, 2023

(30) Foreign Application Priority Data
Jun. 2, 2021 (WO) PCT/CN2021/097848
Apr. 29, 2022 (WO) PCT/CN2022/090237

(51) Int. Cl. C07D 471/14; A61K 31/4985; A61K 31/506; A61K 45/06

(52) U.S. Cl. CPC C07D 471/14; A61K 31/4985; A61K 31/506; A61K 45/06

(57) ABSTRACT

The application describes fused heterocycle derivative compounds, pharmaceutical compositions comprising these compounds, chemical processes for preparing these compounds and their use in the treatment of diseases associated with HBV infection.

This application claims priority to International Application PCT/CN2021/097848, filed on Jun. 2, 2021; and International Application PCT/CN2022/090237, filed on Apr. 29, 2022. Each disclosure is incorporated herein by reference in its entirety.

### FIELD

The application relates to fused heterocyclic derivative compounds, pharmaceutical compositions comprising these compounds, chemical processes for preparing these compounds and their use in the treatment of diseases associated with HBV infection.

### BACKGROUND

Chronic hepatitis B virus (HBV) infection is a significant global health problem, affecting over 5% of the world population (over 350 million people worldwide and 1.25 million individuals in the U.S.).

Despite the availability of a prophylactic HBV vaccine, the burden of chronic HBV infection continues to be a significant unmet worldwide medical problem, due to suboptimal treatment options and sustained rates of new infections in most parts of the developing world. Current treatments do not provide a cure and are limited to only two classes of agents (interferon alpha and nucleoside analogues/inhibitors of the viral polymerase); drug resistance, low efficacy, and tolerability issues limit their impact. The low cure rates of HBV are attributed at least in part to the fact that complete suppression of virus production is difficult to achieve with a single antiviral agent. However, persistent suppression of HBV DNA slows liver disease progression and helps to prevent hepatocellular carcinoma. Current therapy goals for HBV-infected patients are directed to reducing serum HBV DNA to low or undetectable levels, and to ultimately reducing or preventing the development of cirrhosis and hepatocellular carcinoma.

The HBV capsid protein plays essential functions during the viral life cycle. HBV capsid/core proteins form metastable viral particles or protein shells that protect the viral genome during intercellular passage, and also play a central role in viral replication processes, including genome encapsidation, genome replication, and virion morphogenesis and egress. Capsid structures also respond to environmental cues to allow un-coating after viral entry. Consistently, the appropriate timing of capsid assembly and dis-assembly, the appropriate capsid stability and the function of core protein have been found to be critical for viral infectivity.

The crucial function of HBV capsid proteins imposes stringent evolutionary constraints on the viral capsid protein sequence, leading to the observed low sequence variability and high conservation. Consistently, mutations in HBV capsid that disrupt its assembly are lethal, and mutations that perturb capsid stability severely attenuate viral replication. The high functional constraints on the multi-functional HBV core/capsid protein is consistent with a high sequence conservation, as many mutations are deleterious to function. Indeed, the core/capsid protein sequences are >90% identical across HBV genotypes and show only a small number of polymorphic residues. Resistance selection to HBV core/capsid protein binding compounds may therefore be difficult to select without large impacts on virus replication fitness.

Reports describing compounds that bind viral capsids and inhibit replication of HIV, rhinovirus and HBV provide strong pharmacological proof of concept for viral capsid proteins as antiviral drug targets.

There is a need in the art for therapeutic agents that can increase the suppression of virus production and that can treat, ameliorate, and/or prevent HBV infection. Administration of such therapeutic agents to an HBV infected patient, either as monotherapy or in combination with other HBV treatments or ancillary treatments, will lead to significantly reduced virus burden, improved prognosis, diminished progression of the disease and enhanced seroconversion rates.

In view of the clinical importance of HBV, the identification of compounds that can increase the suppression of virus production and that can treat, ameliorate, and/or prevent HBV infection represents an attractive avenue into the development of new therapeutic agents. Such compounds are provided herein.

### SUMMARY

The present disclosure is directed to the general and preferred embodiments defined, respectively, by the independent and dependent claims appended hereto, which are incorporated by reference herein. The present disclosure is directed to compounds capable of capsid assembly modulation. The compounds of the present disclosure may provide a beneficial balance of properties with respect to prior art compounds, e.g. they may display a different profile, display improved solubility, etc.

Thus, in particular, the present disclosure is directed to a compound of Formula (I):

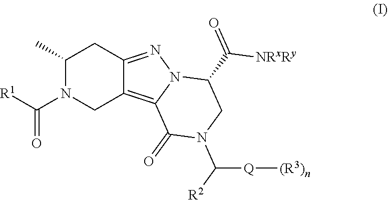

or a stereoisomeric or a tautomeric form thereof, wherein
R¹ is selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₁₋₆alkoxyl, C₃₋₆cycloalkyl and CN, each of C₁₋₆alkyl, C₁₋₆alkoxyl and C₃₋₆cycloalkyl is optionally substituted with 1, 2, 3, 4 or 5 substituents independently selected from the group consisting of halo, hydroxyl and CN;
R² is selected from the group consisting of H, CHF₂, CF₃, C₁₋₆alkyl, C₁₋₆alkylOC₁₋₆alkyl and C₃₋₆cycloalkyl;
Q represents a ring selected from the group consisting of phenyl, 5-membered heteroaryl and 6-membered heteroaryl;
n represents 0, 1, 2 or 3;
each R³ independently represents a substituent selected from the group consisting of CN, C₁₋₆alkyl, C₁₋₆alkoxyl, C₃₋₆cycloalkyl, 5-membered heteroaryl, 6-membered heteroaryl, 4-8 membered heterocyclyl, halo, N(R_{S})₂, S(O)R_{S} and S(O)₂R_{S}, each of C₁₋₆alkyl, C₁₋₆alkoxyl, C₃₋₆cycloalkyl, 5-membered heteroaryl, 6-membered heteroaryl, 4-8 membered heterocyclyl, N(R_{S})₂, S(O)R_{S} and S(O)₂R_{S} is optionally substituted with 1, 2, 3, 4 or 5 substituents, each of said substituents independently selected from the group consisting of halo, hydroxyl, C₁₋₆alkyl and oxo; R_{S} is each independently selected from the group consisting of H, C₁₋₆alkyl, C₁₋₆ alkoxyl and C₃₋₆cycloalkyl;
R^{x} and R^{y} are each independently selected from the group consisting of hydrogen. C₁₋₆alkyl and C₃₋₆cycloalkyl;
   or a pharmaceutically acceptable salt or a solvate thereof.

Further embodiments include pharmaceutically acceptable salts and solvates of compounds of Formula (I), and stereoisomeric and tautomeric forms of the compounds of Formula (I), as well as pharmaceutically acceptable salts thereof.

In embodiments, the compounds of Formula (I) are compounds selected from those species described or exemplified in the detailed description below.

The present disclosure is also directed to pharmaceutical compositions comprising one or more compounds of Formula (I), and pharmaceutically acceptable salts and solvates of compounds of Formula (I). Pharmaceutical compositions may further comprise one or more pharmaceutically acceptable excipients or one or more other agents or therapeutics.

The present disclosure is also directed to methods of using or uses of compounds of Formula (I). In embodiments, compounds of Formula (I) are used to treat or ameliorate hepatitis B viral (HBV) infection, increase the suppression of HBV production, interfere with HBV capsid assembly or other HBV viral replication steps or products thereof. The methods comprise administering to a subject in need of such method an effective amount of at least one compound of Formula (I), and pharmaceutically acceptable salts and solvates of compounds of Formula (I). Additional embodiments of methods of treatment are set forth in the detailed description.

### DETAILED DESCRIPTION

Additional embodiments, features, and advantages of the subject matter of the present disclosure will be apparent from the following detailed description of such disclosure and through its practice. For the sake of brevity, the publications, including patents, cited in this specification are herein incorporated by reference.

In one embodiment, provided herein are compounds of Formula (I),

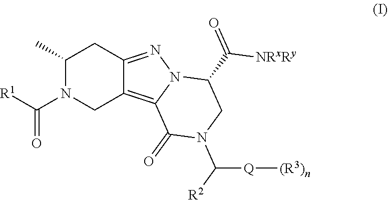

or a stereoisomeric or a tautomeric form thereof, wherein
R¹ is selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₁₋₆alkoxyl, C₃₋₆cycloalkyl and CN, each of C₁₋₆alkyl, C₁₋₆alkoxyl and C₃₋₆cycloalkyl is optionally substituted with 1, 2, 3, 4 or 5 substituents independently selected from the group consisting of halo, hydroxyl and CN;
R² is selected from the group consisting of H, CHF₂, CF₃, C₁₋₆alkyl, C₁₋₆alkylOC₁₋₆alkyl and C₃₋₆cycloalkyl;
Q represents a ring selected from the group consisting of phenyl, 5-membered heteroaryl and 6-membered heteroaryl;
n represents 0, 1, 2 or 3;
each R³ independently represents a substituent selected from the group consisting of CN, C₁₋₆alkyl, C₁₋₆alkoxyl, C₃₋₆cycloalkyl, 5-membered heteroaryl, 6-membered heteroaryl, 4-8 membered heterocyclyl, halo, N(R_{S})₂, S(O)R_{S} and S(O)₂R_{S}, each of C₁₋₆alkyl, C₁₋₆alkoxyl, C₃₋₆cycloalkyl, 5-membered heteroaryl, 6-membered heteroaryl, 4-8 membered heterocyclyl, N(R_{S})₂, S(O)R_{S} and S(O)₂R_{S} is optionally substituted with 1, 2, 3, 4 or 5 substituents, each of said substituents independently selected from the group consisting of halo, hydroxyl, C₁₋₆alkyl and oxo; R_{S} is each independently selected from the group consisting of H, C₁₋₆alkyl, C₁₋₆alkoxyl and C₃₋₆cycloalkyl;
R^{x} and R^{y} are each independently selected from the group consisting of hydrogen, C₁₋₆alkyl, and C₃₋₆cycloalkyl;
   or a pharmaceutically acceptable salt or a solvate thereof.

In an embodiment, the following compound is excluded from the compound of Formula (I) herein:

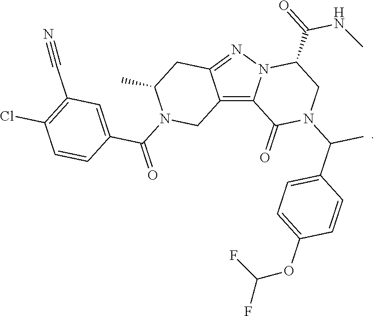 [SMILES: CNC(=O)[C@@H]1CN(C(C)c2ccc(OC(F)F)cc2)C(=O)c2c3c(nn21)C[C@@H](C)N(C(=O)c1ccc(Cl)c(C#N)c1)C3]

The following compounds are also excluded:

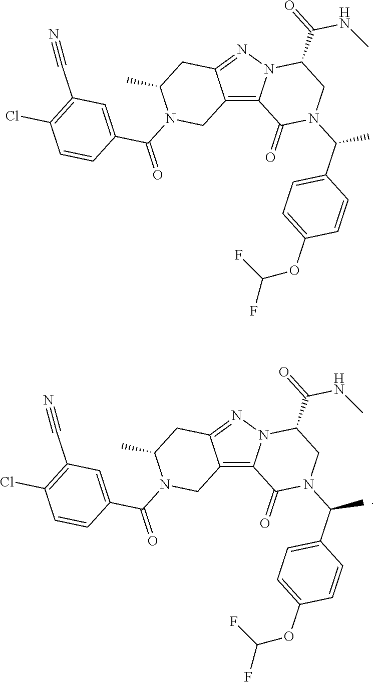 [SMILES: CNC(=O)[C@@H]1CN([C@H](C)c2ccc(OC(F)F)cc2)C(=O)c2c3c(nn21)C[C@@H](C)N(C(=O)c1ccc(Cl)c(C#N)c1)C3 ; CNC(=O)[C@@H]1CN([C@@H](C)c2ccc(OC(F)F)cc2)C(=O)c2c3c(nn21)C[C@@H](C)N(C(=O)c1ccc(Cl)c(C#N)c1)C3]

In an embodiment, R¹ is selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₁₋₆alkoxyl, C₃₋₆cycloalkyl and CN. Each of C₁₋₆alkyl, C₁₋₆alkoxyl and C₃₋₆cycloalkyl is optionally substituted with 1, 2, 3, 4 or 5 (e.g., 1, 2 or 3) substituents independently selected from the group consisting of halo (e.g., F).

In a preferable embodiment, R¹ is selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₃₋₆cycloalkyl, CN, CF₃, CHF₂, OCHF₂ and OCF₃.

In another embodiment, R¹ is selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₃₋₆cycloalkyl, CF₃, CHF₂, OCHF₂, CN and OCF₃. In yet another embodiment, R¹ is selected from the group consisting of phenyl, 5-membered heteroaryl and 6-membered heteroaryl, each of which is substituted with 1, 2 or 3 substituents, each of said substituents independently selected from the group consisting of halo, CN and CF₃.

In an embodiment, R¹ is a ring selected from the group consisting of phenyl, pyridyl, pyrimidinyl, pyridazinyl and pyrazinyl, each of which is substituted with 1, 2 or 3 substituents, each of said substituents independently selected from the group consisting of halo, C₁₋₆alkyl, C₃₋₆cycloalkyl, CN, CF₃, CHF₂, OCHF₂, CN and OCF₃. In a preferable embodiment, R¹ is a ring selected from the group consisting of phenyl, pyridyl, pyrimidinyl, pyridazinyl and pyrazinyl, each of which is substituted with 1, 2 or 3 substituents, each of said substituents independently selected from the group consisting of halo, C₁₋₆alkyl, C₃₋₆cycloalkyl, CF₃, CHF₂, OCHF₂, CN and OCF₃; more preferably each of said substituents independently selected from the group consisting of halo, C₁₋₆alkyl, CF₃, CN and CHF₂; most preferably selected from the group consisting of halo, CN and CF₃.

In a yet preferable embodiment, R¹ is a ring of phenyl. In a further embodiment, R¹ is a ring of phenyl, which is substituted with 1, 2 or 3 substituents, each of said substituents independently selected from the group consisting of halo, CN and CF₃.

In an embodiment, the number of substituents on R¹ is 1 or 2, preferably 2.

In an embodiment, the structural unit

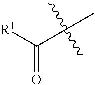

in Formula (I) of the present disclosure satisfies Formula (Ia)

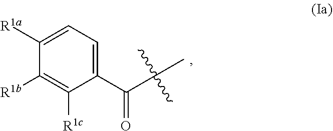

wherein R^{1a}, R^{1b}, and R^{1c}, each independently are selected from the group consisting of hydrogen, halo, C₁₋₆alkyl, C₃₋₆cycloalkyl, CN, CF₃, CHF₂, OCHF₂, CN and OCF₃, with at least one of R^{1a}, R^{1b}, and R^{1c} not being hydrogen;
preferably, wherein R^{1a}, R^{1b}, and R^{1c}, each independently are selected from the group consisting of hydrogen, halo, C₁₋₆alkyl, C₃₋₆cycloalkyl, CF₃, CHF₂, OCHF₂, CN and OCF₃, with at least one of R^{1a}, R^{1b}, and R^{1c} not being hydrogen.

In a specific embodiment, R^{1a} and R^{1b} are independently selected from the group consisting of halo, CN, CHF₂, CF₃, OCHF₂ and OCF₃, and R^{1c} is hydrogen. In another specific embodiment, R^{1a} and R^{1b} are independently selected from the group consisting of halo, CN, CHF₂, and CF₃, and wherein R^{1c} is hydrogen.

In a preferable embodiment, R^{1a} is halo, R^{1b} is selected from the group consisting of halo, CN, CHF₂ and CF₃, and R^{1c} is hydrogen. In a further embodiment, R^{1a} is halo, R^{1b} is halo and R^{1c} is hydrogen.

Preferably, the halo is Cl.

In an embodiment, R² is selected from the group consisting of CHF₂, CF₃, C₁₋₆alkyl, C₁₋₆alkylOC₁₋₆alkyl and C₃₋₆-cycloalkyl; and the structure of Formula (I) has Formula (I-1) or Formula (I-2)

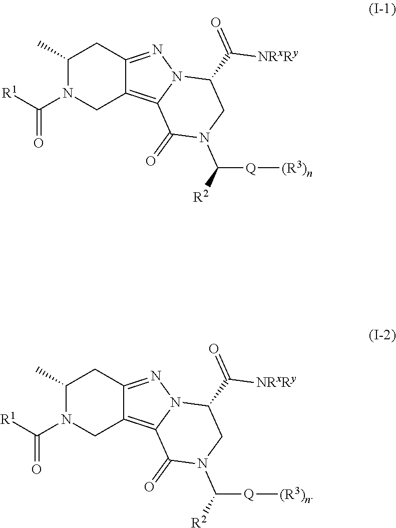

In a further embodiment, R² is C₁₋₆alkyl, preferably methyl or ethyl.

In another embodiment, R² is H.

In an embodiment, Q represents a ring selected from the group consisting of phenyl and 6-membered heteroaryl. In a further embodiment, Q is a ring of 6-membered heteroaryl, which is selected from the group consisting of pyridyl, pyrimidinyl, pyridazinyl and pyrazinyl.

In another embodiment, Q represents a ring selected from the group consisting of 5-membered heteroaryl. In a specific embodiment, Q is a ring selected from the group consisting of pyrazolyl, imidazolyl, and thiazolyl.

In an embodiment, the structural unit

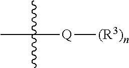

in Formula (I) of the present disclosure satisfies Formula (Ib)

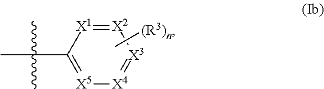

wherein
all of X¹, X², X³, X⁴ and X⁵ are CH.

In the embodiment where all of X¹, X², X³, X⁴ and X⁵ are CH, the ring is a phenyl ring.

In such an embodiment, R³(s) is/are each independently linked to n of X¹-X⁵. Preferably, n is 1 or 2, and R³ (s) is/are is linked to one or two of X¹-X⁵. In a specific embodiment where n is 1, R³ is linked to X³. In an alternative embodiment where n is 2, R³s are linked to two of X¹—X⁵. In an exemplary embodiment where n is 2, one R³ is linked to X³ and other is linked to X¹ or X². It should be noted that when there are more than one R³, they are independently selected and thus may be identical or different.

In an embodiment, the structural unit

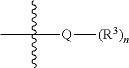

in Formula (I) of the present disclosure satisfies Formula (Ib)

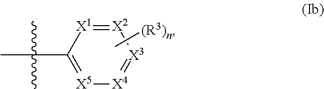

wherein
one or two of X¹, X², X³, X⁴ and X⁵ are N, and rest of them are CH.

In the embodiment where one or two of X¹, X², X³, X⁴ and X⁵ are N, and rest of them are CH, the ring is a 6-membered heteroaryl. In such an embodiment, R³(s) are each independently linked to n of X¹-X⁵, which are CH. Preferably, n is 1 or 2 and R³(s) is/are linked to one or two of X¹-X⁵ which is/are CH. In a specific embodiment where n is 1, R³ is linked to X³ which is CH. In an alternative embodiment where n is 2, R³s are linked to two of X¹-X⁵. In an exemplary embodiment where n is 2, one R³ is linked to X³ (which is CH) and other is linked to X¹ (which is CH), X² (which is CH), X⁴ (which is CH) or X⁵ (which is CH). It should be noted that when there are more than one R³, they are independently selected and thus may be identical or different.

In a specific embodiment, one of X¹-X⁵ is N, and rest of them are CH. In another specific embodiment, two of X¹-X⁵ are N, and rest of them are CH.

In a particular embodiment,
X¹ is N, and X², X³, X⁴, X⁵ are CH; or
X² is N, and X¹, X³, X⁴, X⁵ are CH; or
X³ is N, and X¹, X², X⁴, X⁵ are CH; or
X¹ and X² are N, and X³, X⁴, X⁵ are CH; or
X¹ and X³ are N, and X², X⁴, X⁵ are CH; or
X¹ and X⁴ are N, and X², X³, X⁵ are CH; or
X¹ and X⁵ are N, and X², X³, X⁴ are CH; or
X² and X³ are N, and X¹, X⁴, X⁵ are CH; or
X² and X⁴ are N, and X¹, X³, X⁵ are CH.

In another particular embodiment,
both of X¹ and X² are N, and X⁴ and X⁵ are CH; or
both of X² and X⁴ are N, and X¹ and X⁵ are CH; or
both of X¹ and X⁴ are N, and X² and X⁵ are CH; or
both of X¹ and X⁵ are N, and X² and X⁴ are CH.

In a specific embodiment, R³s is/are linked to one or two of X¹-X⁵, which is/are CH.

In an embodiment, the structural unit

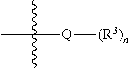

in Formula (I) of the present disclosure satisfies Formula (Ic)

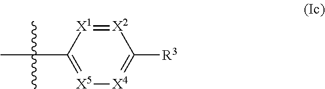

wherein
all of X¹, X², X⁴ and X⁵ are CH.

In a specific embodiment, one or two of X¹, X², X⁴, X⁵ is/are optionally substituted by another R³(s). It should be noted that when there are more than one R³, they are independently selected and thus may be identical or different.

In an embodiment, the structural unit

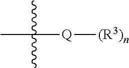

in Formula (I) of the present disclosure satisfies Formula (Ic)

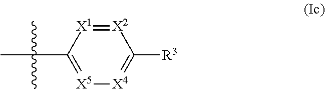

wherein
one of X² and X⁴ is N, and the other is CH.

In a specific embodiment of the above Formula (Ic),
i) one of X¹ and X⁵ is N, and rest of them is CH; or
ii) both of X¹ and X⁵ are CH.

In a specific embodiment, one or two of X¹, X², X⁴, X⁵ (which is/are CH) is/are optionally substituted by another R³(s). It should be noted that when there are more than one R³, they are independently selected and thus may be identical or different.

In an embodiment, the structural unit

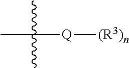

in Formula (I) of the present disclosure satisfies Formula (Ic)

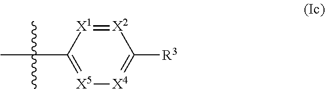

wherein
both of X² and X⁴ are N.

In a specific embodiment of the above Formula (Ic),
both of X¹ and X⁵ are CH.

In a specific embodiment, one or two of X¹, X⁵ (which is/are CH) is/are optionally substituted by another R³(s). It should be noted that when there are more than one R³, they are independently selected and thus may be identical or different.

In an embodiment, the structural unit,

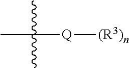

in in Formula (I) of the present disclosure satisfies Formula (Ic)

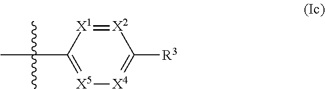

wherein
one of X¹ and X⁵ is N, and the other is CH.

In a specific embodiment of the above Formula (Ic),
i) one of X² and X⁴ is N, and rest of them is CH; or
ii) both of X² and X⁴ are CH.

In a specific embodiment, one or two of X¹, X², X⁴, X⁵ (which is/are CH) is/are optionally substituted by another R³(s). It should be noted that when there are more than one R³, they are independently selected and thus may be identical or different.

In an embodiment, the structural unit

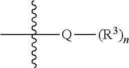

in Formula (I) of the present disclosure satisfies Formula (Ic)

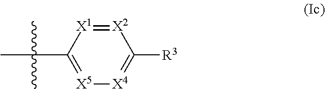

wherein
both of X¹ and X⁵ are N.

In a specific embodiment of the above Formula (Ic),
both of X² and X⁴ are CH.

In a specific embodiment, one or two of X², X⁴ (which is/are CH) is/are optionally substituted by another R³(s). It should be noted that when there are more than one R³, they are independently selected and thus may be identical or different.

In an embodiment, the structural unit

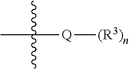

in Formula (I) satisfies Formula (Ic)

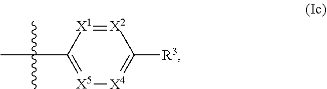

wherein
all of X¹, X², X⁴ and X⁵ are CH; or
X² is N, and X¹, X⁴ and X⁵ are CH; or
X¹ is N, and X², X⁴ and X⁵ are CH.

In a specific embodiment, the structural unit

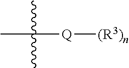

in Formula (I) satisfies Formula (Ic)

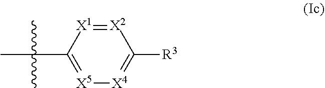

both of X¹ and X² are N, and X⁴ and X⁵ are CH; or
both of X² and X⁴ are N, and X¹ and X⁵ are CH; or
both of X¹ and X⁴ are N, and X² and X⁵ are CH; or
both of X¹ and X⁵ are N, and X² and X⁴ are CH.

In an embodiment, the structural unit

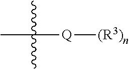

in Formula (I) of the present disclosure satisfies Formula (Ib′)

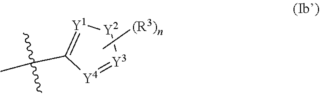

wherein one or two of Y¹, Y², Y³ and Y⁴ are N (or NH) or S, and rest of them are CH.

In a specific embodiment, two of Y¹, Y², Y³ and Y⁴ are N (or NH) or S, and rest of them are CH. In a preferable embodiment, Y¹ and Y² are N (or NH). In another preferable embodiment, Y¹ is N (or NH) and Y⁴ is S; or Y¹ is S and Y⁴ is N (or NH).

In the embodiment where one or two of Y¹, Y², Y³ and Y⁴ are N (or NH) or S, and rest of them are CH, the ring is a 5-membered heteroaryl. In such an embodiment, R³(s) are each independently linked to one or more of Y¹-Y⁴. Preferably, n is 1 or 2 and R³(s) is/are linked to one or two of Y¹-Y⁴.

In an embodiment where n is 1, R³ is linked to Y². In an embodiment where n is 1, R³ is linked to Y¹. In another embodiment, Y¹ and Y² are N (or NH); or Y or Y² is N (or NH); or Y¹ is N and Y⁴ is S; or Y⁴ is N and Y¹ is S.

In a specific embodiment, Q is a ring of

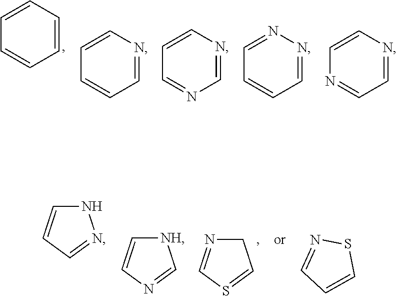

(where H may be absence due to substitution or connection to other part of the molecule), which is substituted with n R³.

In a specific embodiment, the CH or NH moiety as described may be optionally substituted, for example, by R³.

It should be noted that when a CH or NH moiety is present, for example within a ring like phenyl or heteroaryl, the “H” may be absent due to substitution or connection to other part of the molecule. Similarly, when a C or N moiety is mentioned, for example within a ring like phenyl or heteroaryl, a H could be present such that a stable compound structure is satisfied, and the corresponding atom or radical may be described only as C or N. For example, in the Formulae like (I), (I-1), (I-2), (Ib), (Ib′), (Ic) etc, when X (e.g., X¹, X², X³, X⁴, X⁵) is described as CH or NH, it means the group is unsubstituted or optionally substituted or connected to other part of the molecule, provided that a stable compound is achieved. Likewise, when C or N is described, “H” or optional substitution/connection may be added such that a stable compound is satisfied.

In an embodiment, n is 1, 2, or 3, e.g., 1 or 2. It should be noted that when there are more than one R³, they are independently selected and thus may be identical or different. When n is 0, it means Q is not substituted, or all R³ groups are H.

In an embodiment, halo is F, Cl or Br, preferably Cl.

In an embodiment, R³ independently represents a substituent selected from the group consisting of CN, C₁₋₆alkyl, C₁₋₆alkoxyl, C₃₋₆cycloalkyl, 5-membered heteroaryl, 4-8 membered heterocyclyl, halo, N(R_{S})₂, S(O)R_{S} and S(O)₂R_{S}, each of C₁₋₆alkyl, C₁₋₆alkoxyl, C₃₋₆cycloalkyl, 5-membered heteroaryl, 4-8 membered heterocyclyl, N(R_{S})₂, S(O)R_{S} and S(O)₂R_{S} is optionally substituted with 1, 2, 3, 4 or 5 substituents, each of said substituents independently selected from the group consisting of halo, hydroxyl, and C₁₋₆alkyl. R_{S} is each independently selected from the group consisting of H, C₁₋₆alkyl, C₁₋₆alkoxyl and C₃₋₆cycloalkyl.

Preferably, the number of substitutes on any of C₁₋₆alkyl, C₁₋₆alkoxyl, C₃₋₆cycloalkyl, 5-membered heteroaryl, 6-membered heteroaryl, 4-8 membered heterocyclyl, N(R_{S})₂, S(O)R_{S} and S(O)₂R_{S} is 1, 2 or 3.

In a specific embodiment, R³ is independently selected from the group consisting of halo, CH₃,

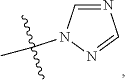 [SMILES: *n1cncn1]

C(CH₃)₂OH, CH₂CF₃, CF₃, OCHF₂, SO₂CH₃

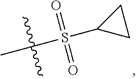 [SMILES: *S(=O)(=O)C1CC1]

### OCH₃, CN,

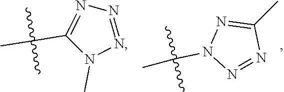

cyclopropyl, NHCH₂CF₃,

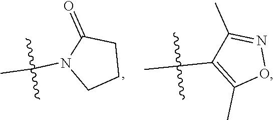

### CHF₂, and

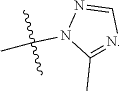 [SMILES: *n1ncnc1C]

In an embodiment, one of R^{x} and R^{Y} is hydrogen, and the other is C₁₋₆alkyl or C₃₋₆cycloalkyl, preferably methyl or C₃cycloalkyl. In another embodiment, R^{x} and R^{Y} are hydrogen. In yet another embodiment, R^{x} and R^{Y} are each independently selected from the group consisting of C₁₋₆alkyl, preferably methyl. In yet another embodiment, R^{x} and R^{Y} are each independently selected from the group consisting of C₁₋₆alkyl (preferably methyl) and C₃₋₆cycloalkyl, (preferably C₃cycloalkyl).

In an embodiment, the heteroaryl (such as, 5-membered heteroaryl or 6-membered heteroaryl) may contain at least one (e.g., 1, 2 or 3, preferably 1 or 2) heteroatoms independently selected from the group consisting of N, O and S, preferably N and S.

In an embodiment, the heterocyclyl (such as, 4-8 membered heterocyclyl) may contain at least one (e.g., 1, 2 or 3, preferably 1 or 2) heteroatoms independently selected from the group consisting of N, O and S, preferably N and S.

A further embodiment of the present disclosure is a compound selected from the group consisting of the compounds described in Table 1 and Table 2 below, a stereoisomer or tautomer thereof, or a pharmaceutically acceptable salt thereof.

TABLE 1

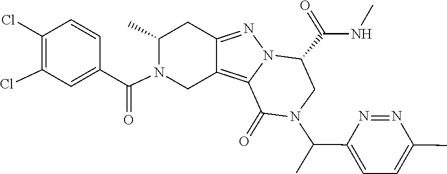 [SMILES: CNC(=O)[C@@H]1CN(C(C)c2ccc(C)nn2)C(=O)c2c3c(nn21)C[C@@H](C)N(C(=O)c1ccc(Cl)c(Cl)c1)C3]

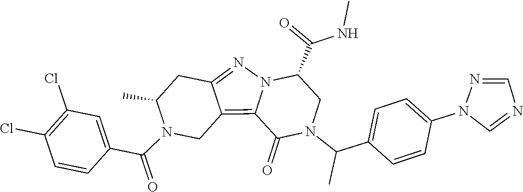 [SMILES: CNC(=O)[C@@H]1CN(C(C)c2ccc(-n3cncn3)cc2)C(=O)c2c3c(nn21)C[C@@H](C)N(C(=O)c1ccc(Cl)c(Cl)c1)C3]

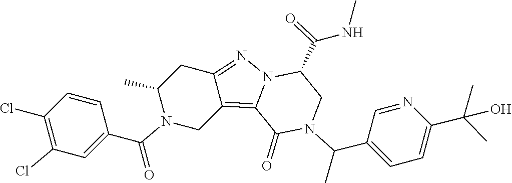 [SMILES: CNC(=O)[C@@H]1CN(C(C)c2ccc(C(C)(C)O)nc2)C(=O)c2c3c(nn21)C[C@@H](C)N(C(=O)c1ccc(Cl)c(Cl)c1)C3]

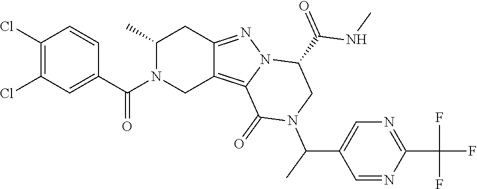 [SMILES: CNC(=O)[C@@H]1CN(C(C)c2cnc(C(F)(F)F)nc2)C(=O)c2c3c(nn21)C[C@@H](C)N(C(=O)c1ccc(Cl)c(Cl)c1)C3]

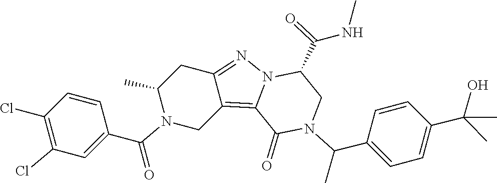 [SMILES: CNC(=O)[C@@H]1CN(C(C)c2ccc(C(C)(C)O)cc2)C(=O)c2c3c(nn21)C[C@@H](C)N(C(=O)c1ccc(Cl)c(Cl)c1)C3]

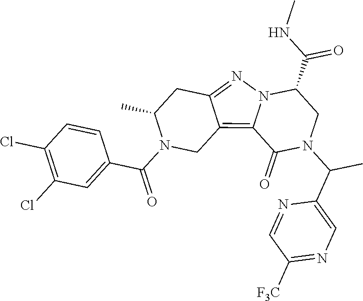 [SMILES: CNC(=O)[C@@H]1CN(C(C)c2cnc(C(F)(F)F)cn2)C(=O)c2c3c(nn21)C[C@@H](C)N(C(=O)c1ccc(Cl)c(Cl)c1)C3]

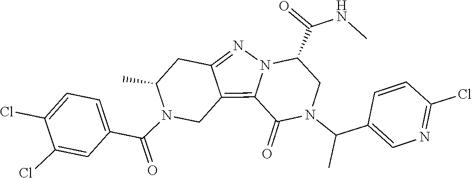 [SMILES: CNC(=O)[C@@H]1CN(C(C)c2ccc(Cl)nc2)C(=O)c2c3c(nn21)C[C@@H](C)N(C(=O)c1ccc(Cl)c(Cl)c1)C3]

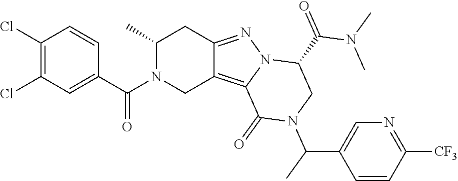 [SMILES: CC(c1ccc(C(F)(F)F)nc1)N1C[C@@H](C(=O)N(C)C)n2nc3c(c2C1=O)CN(C(=O)c1ccc(Cl)c(Cl)c1)[C@H](C)C3]

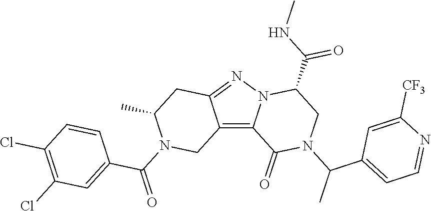 [SMILES: CNC(=O)[C@@H]1CN(C(C)c2ccnc(C(F)(F)F)c2)C(=O)c2c3c(nn21)C[C@@H](C)N(C(=O)c1ccc(Cl)c(Cl)c1)C3]

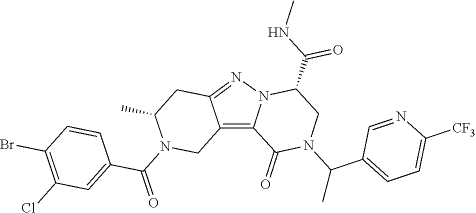 [SMILES: CNC(=O)[C@@H]1CN(C(C)c2ccc(C(F)(F)F)nc2)C(=O)c2c3c(nn21)C[C@@H](C)N(C(=O)c1ccc(Br)c(Cl)c1)C3]

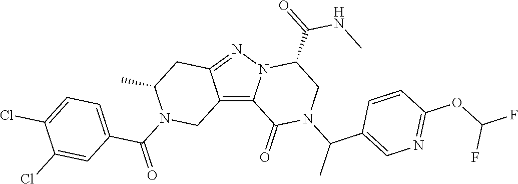 [SMILES: CNC(=O)[C@@H]1CN(C(C)c2ccc(OC(F)F)nc2)C(=O)c2c3c(nn21)C[C@@H](C)N(C(=O)c1ccc(Cl)c(Cl)c1)C3]

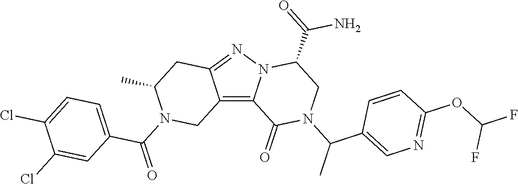 [SMILES: CC(c1ccc(OC(F)F)nc1)N1C[C@@H](C(N)=O)n2nc3c(c2C1=O)CN(C(=O)c1ccc(Cl)c(Cl)c1)[C@H](C)C3]

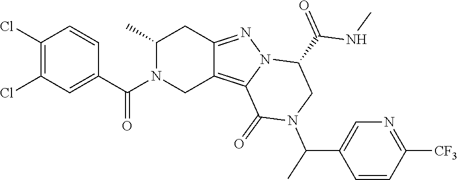 [SMILES: CNC(=O)[C@@H]1CN(C(C)c2ccc(C(F)(F)F)nc2)C(=O)c2c3c(nn21)C[C@@H](C)N(C(=O)c1ccc(Cl)c(Cl)c1)C3]

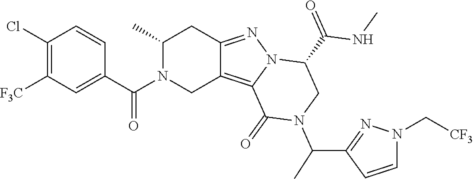 [SMILES: CNC(=O)[C@@H]1CN(C(C)c2ccn(CC(F)(F)F)n2)C(=O)c2c3c(nn21)C[C@@H](C)N(C(=O)c1ccc(Cl)c(C(F)(F)F)c1)C3]

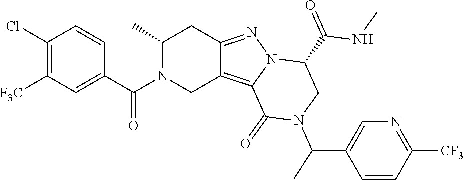 [SMILES: CNC(=O)[C@@H]1CN(C(C)c2ccc(C(F)(F)F)nc2)C(=O)c2c3c(nn21)C[C@@H](C)N(C(=O)c1ccc(Cl)c(C(F)(F)F)c1)C3]

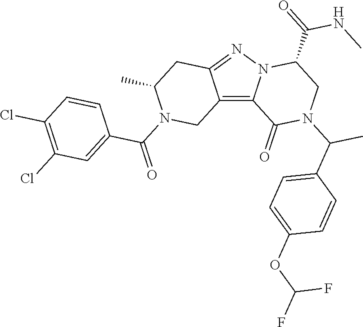 [SMILES: CNC(=O)[C@@H]1CN(C(C)c2ccc(OC(F)F)cc2)C(=O)c2c3c(nn21)C[C@@H](C)N(C(=O)c1ccc(Cl)c(Cl)c1)C3]

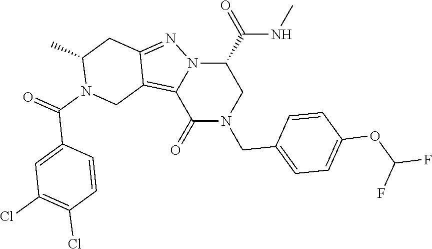 [SMILES: CNC(=O)[C@@H]1CN(Cc2ccc(OC(F)F)cc2)C(=O)c2c3c(nn21)C[C@@H](C)N(C(=O)c1ccc(Cl)c(Cl)c1)C3]

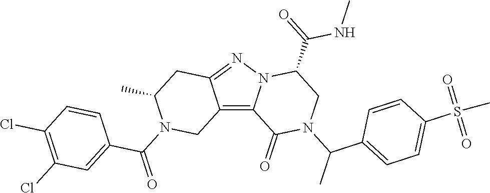 [SMILES: CNC(=O)[C@@H]1CN(C(C)c2ccc(S(C)(=O)=O)cc2)C(=O)c2c3c(nn21)C[C@@H](C)N(C(=O)c1ccc(Cl)c(Cl)c1)C3]

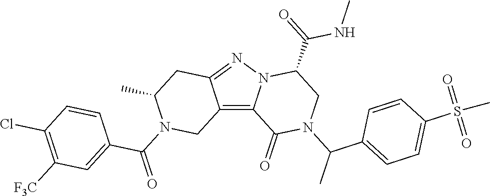 [SMILES: CNC(=O)[C@@H]1CN(C(C)c2ccc(S(C)(=O)=O)cc2)C(=O)c2c3c(nn21)C[C@@H](C)N(C(=O)c1ccc(Cl)c(C(F)(F)F)c1)C3]

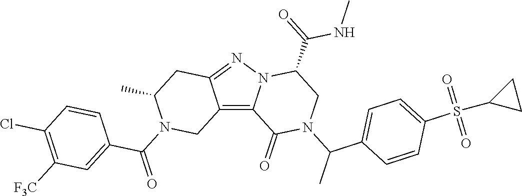 [SMILES: CNC(=O)[C@@H]1CN(C(C)c2ccc(S(=O)(=O)C3CC3)cc2)C(=O)c2c3c(nn21)C[C@@H](C)N(C(=O)c1ccc(Cl)c(C(F)(F)F)c1)C3]

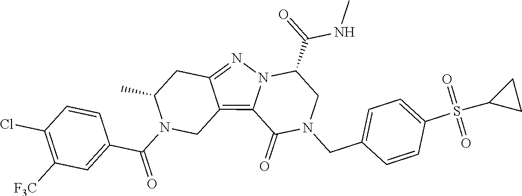 [SMILES: CNC(=O)[C@@H]1CN(Cc2ccc(S(=O)(=O)C3CC3)cc2)C(=O)c2c3c(nn21)C[C@@H](C)N(C(=O)c1ccc(Cl)c(C(F)(F)F)c1)C3]

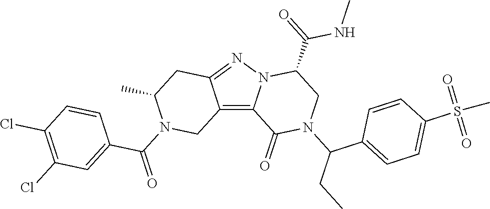 [SMILES: CCC(c1ccc(S(C)(=O)=O)cc1)N1C[C@@H](C(=O)NC)n2nc3c(c2C1=O)CN(C(=O)c1ccc(Cl)c(Cl)c1)[C@H](C)C3]

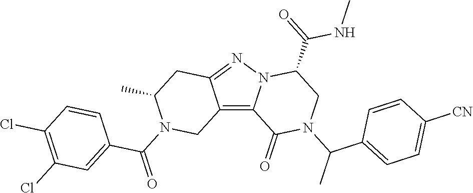 [SMILES: CNC(=O)[C@@H]1CN(C(C)c2ccc(C#N)cc2)C(=O)c2c3c(nn21)C[C@@H](C)N(C(=O)c1ccc(Cl)c(Cl)c1)C3]

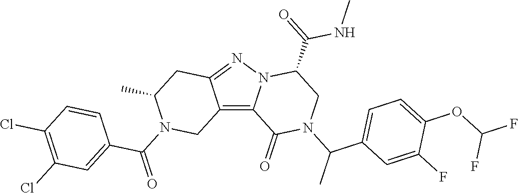 [SMILES: CNC(=O)[C@@H]1CN(C(C)c2ccc(OC(F)F)c(F)c2)C(=O)c2c3c(nn21)C[C@@H](C)N(C(=O)c1ccc(Cl)c(Cl)c1)C3]

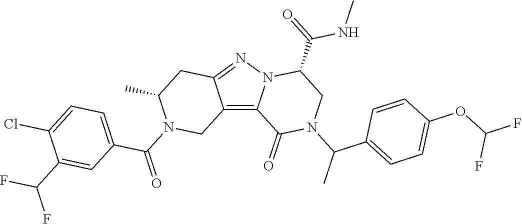 [SMILES: CNC(=O)[C@@H]1CN(C(C)c2ccc(OC(F)F)cc2)C(=O)c2c3c(nn21)C[C@@H](C)N(C(=O)c1ccc(Cl)c(C(F)F)c1)C3]

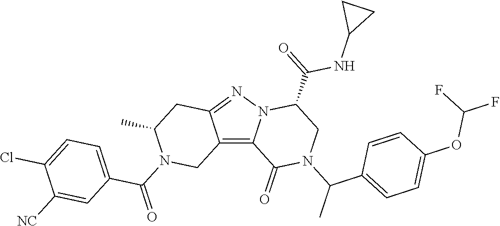 [SMILES: CC(c1ccc(OC(F)F)cc1)N1C[C@@H](C(=O)NC2CC2)n2nc3c(c2C1=O)CN(C(=O)c1ccc(Cl)c(C#N)c1)[C@H](C)C3]

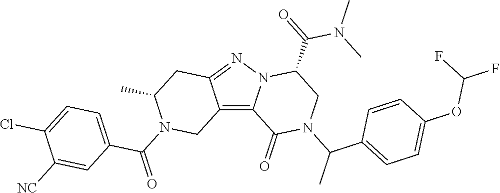 [SMILES: CC(c1ccc(OC(F)F)cc1)N1C[C@@H](C(=O)N(C)C)n2nc3c(c2C1=O)CN(C(=O)c1ccc(Cl)c(C#N)c1)[C@H](C)C3]

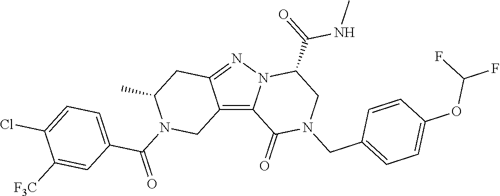 [SMILES: CNC(=O)[C@@H]1CN(Cc2ccc(OC(F)F)cc2)C(=O)c2c3c(nn21)C[C@@H](C)N(C(=O)c1ccc(Cl)c(C(F)(F)F)c1)C3]

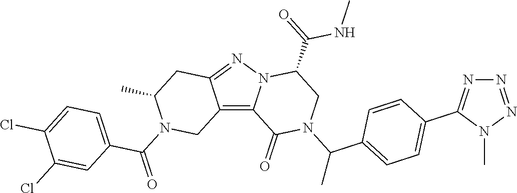 [SMILES: CNC(=O)[C@@H]1CN(C(C)c2ccc(-c3nnnn3C)cc2)C(=O)c2c3c(nn21)C[C@@H](C)N(C(=O)c1ccc(Cl)c(Cl)c1)C3]

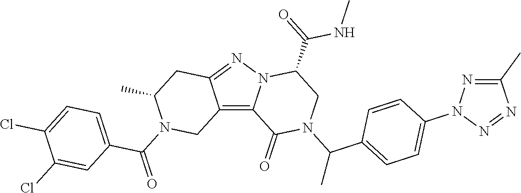 [SMILES: CNC(=O)[C@@H]1CN(C(C)c2ccc(-n3nnc(C)n3)cc2)C(=O)c2c3c(nn21)C[C@@H](C)N(C(=O)c1ccc(Cl)c(Cl)c1)C3]

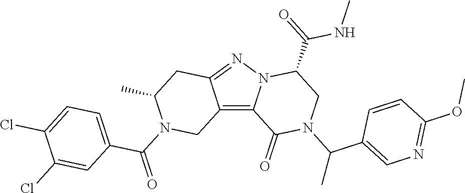 [SMILES: CNC(=O)[C@@H]1CN(C(C)c2ccc(OC)nc2)C(=O)c2c3c(nn21)C[C@@H](C)N(C(=O)c1ccc(Cl)c(Cl)c1)C3]

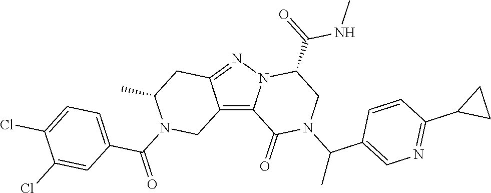 [SMILES: CNC(=O)[C@@H]1CN(C(C)c2ccc(C3CC3)nc2)C(=O)c2c3c(nn21)C[C@@H](C)N(C(=O)c1ccc(Cl)c(Cl)c1)C3]

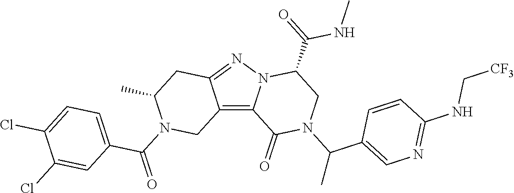 [SMILES: CNC(=O)[C@@H]1CN(C(C)c2ccc(NCC(F)(F)F)nc2)C(=O)c2c3c(nn21)C[C@@H](C)N(C(=O)c1ccc(Cl)c(Cl)c1)C3]

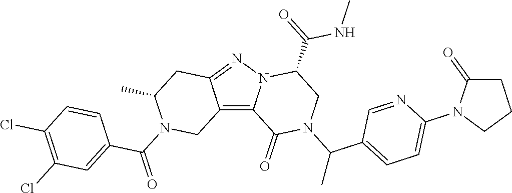 [SMILES: CNC(=O)[C@@H]1CN(C(C)c2ccc(N3CCCC3=O)nc2)C(=O)c2c3c(nn21)C[C@@H](C)N(C(=O)c1ccc(Cl)c(Cl)c1)C3]

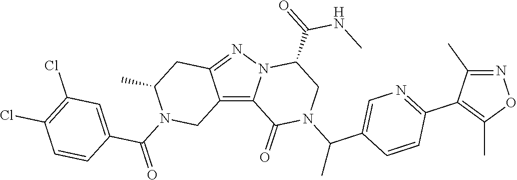 [SMILES: CNC(=O)[C@@H]1CN(C(C)c2ccc(-c3c(C)noc3C)nc2)C(=O)c2c3c(nn21)C[C@@H](C)N(C(=O)c1ccc(Cl)c(Cl)c1)C3]

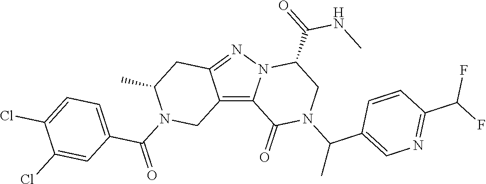 [SMILES: CNC(=O)[C@@H]1CN(C(C)c2ccc(C(F)F)nc2)C(=O)c2c3c(nn21)C[C@@H](C)N(C(=O)c1ccc(Cl)c(Cl)c1)C3]

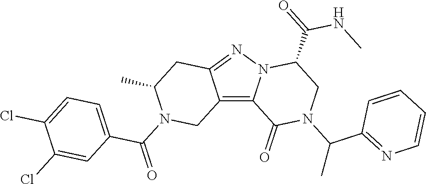 [SMILES: CNC(=O)[C@@H]1CN(C(C)c2ccccn2)C(=O)c2c3c(nn21)C[C@@H](C)N(C(=O)c1ccc(Cl)c(Cl)c1)C3]

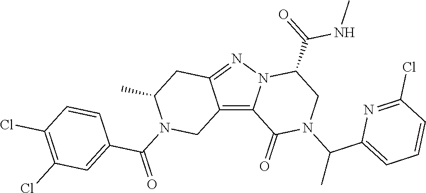 [SMILES: CNC(=O)[C@@H]1CN(C(C)c2cccc(Cl)n2)C(=O)c2c3c(nn21)C[C@@H](C)N(C(=O)c1ccc(Cl)c(Cl)c1)C3]

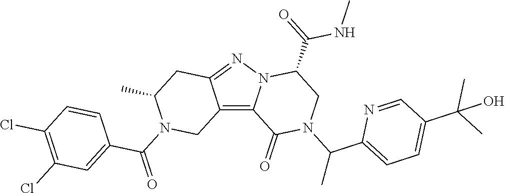 [SMILES: CNC(=O)[C@@H]1CN(C(C)c2ccc(C(C)(C)O)cn2)C(=O)c2c3c(nn21)C[C@@H](C)N(C(=O)c1ccc(Cl)c(Cl)c1)C3]

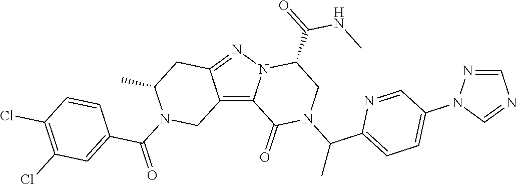 [SMILES: CNC(=O)[C@@H]1CN(C(C)c2ccc(-n3cncn3)cn2)C(=O)c2c3c(nn21)C[C@@H](C)N(C(=O)c1ccc(Cl)c(Cl)c1)C3]

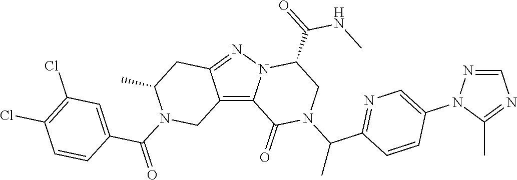 [SMILES: CNC(=O)[C@@H]1CN(C(C)c2ccc(-n3ncnc3C)cn2)C(=O)c2c3c(nn21)C[C@@H](C)N(C(=O)c1ccc(Cl)c(Cl)c1)C3]

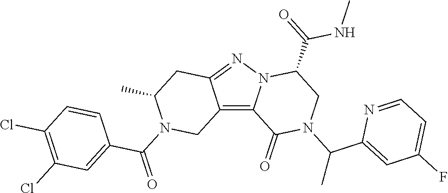 [SMILES: CNC(=O)[C@@H]1CN(C(C)c2cc(F)ccn2)C(=O)c2c3c(nn21)C[C@@H](C)N(C(=O)c1ccc(Cl)c(Cl)c1)C3]

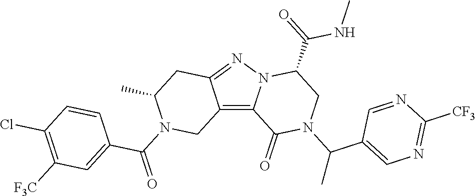 [SMILES: CNC(=O)[C@@H]1CN(C(C)c2cnc(C(F)(F)F)nc2)C(=O)c2c3c(nn21)C[C@@H](C)N(C(=O)c1ccc(Cl)c(C(F)(F)F)c1)C3]

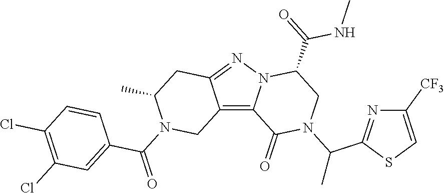 [SMILES: CNC(=O)[C@@H]1CN(C(C)c2nc(C(F)(F)F)cs2)C(=O)c2c3c(nn21)C[C@@H](C)N(C(=O)c1ccc(Cl)c(Cl)c1)C3]

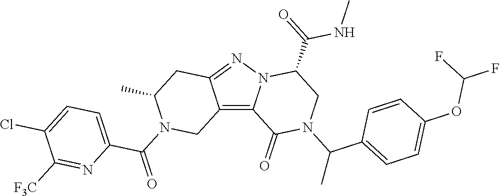 [SMILES: CNC(=O)[C@@H]1CN(C(C)c2ccc(OC(F)F)cc2)C(=O)c2c3c(nn21)C[C@@H](C)N(C(=O)c1ccc(Cl)c(C(F)(F)F)n1)C3]

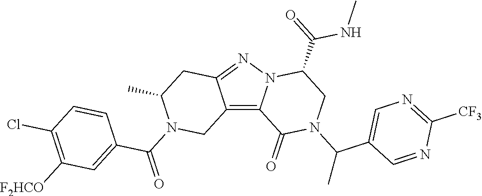 [SMILES: CNC(=O)[C@@H]1CN(C(C)c2cnc(C(F)(F)F)nc2)C(=O)c2c3c(nn21)C[C@@H](C)N(C(=O)c1ccc(Cl)c(OC(F)F)c1)C3]

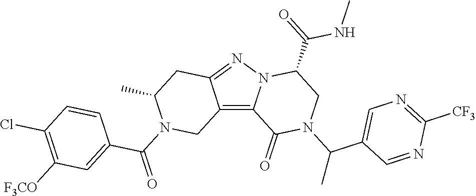 [SMILES: CNC(=O)[C@@H]1CN(C(C)c2cnc(C(F)(F)F)nc2)C(=O)c2c3c(nn21)C[C@@H](C)N(C(=O)c1ccc(Cl)c(OC(F)(F)F)c1)C3]

TABLE 2

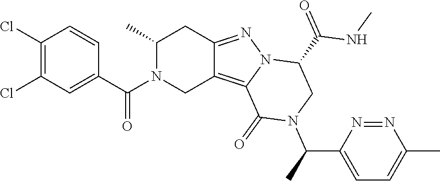 [SMILES: CNC(=O)[C@@H]1CN([C@H](C)c2ccc(C)nn2)C(=O)c2c3c(nn21)C[C@@H](C)N(C(=O)c1ccc(Cl)c(Cl)c1)C3]

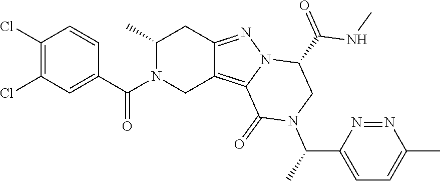 [SMILES: CNC(=O)[C@@H]1CN([C@@H](C)c2ccc(C)nn2)C(=O)c2c3c(nn21)C[C@@H](C)N(C(=O)c1ccc(Cl)c(Cl)c1)C3]

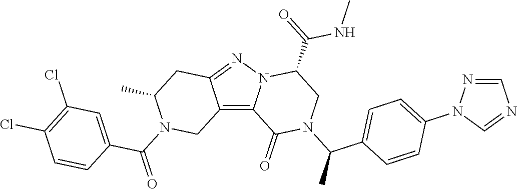 [SMILES: CNC(=O)[C@@H]1CN([C@H](C)c2ccc(-n3cncn3)cc2)C(=O)c2c3c(nn21)C[C@@H](C)N(C(=O)c1ccc(Cl)c(Cl)c1)C3]

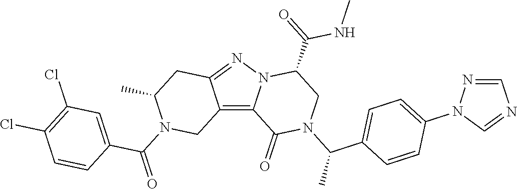 [SMILES: CNC(=O)[C@@H]1CN([C@@H](C)c2ccc(-n3cncn3)cc2)C(=O)c2c3c(nn21)C[C@@H](C)N(C(=O)c1ccc(Cl)c(Cl)c1)C3]

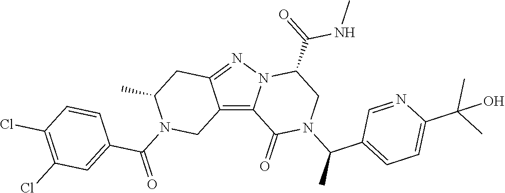 [SMILES: CNC(=O)[C@@H]1CN([C@H](C)c2ccc(C(C)(C)O)nc2)C(=O)c2c3c(nn21)C[C@@H](C)N(C(=O)c1ccc(Cl)c(Cl)c1)C3]

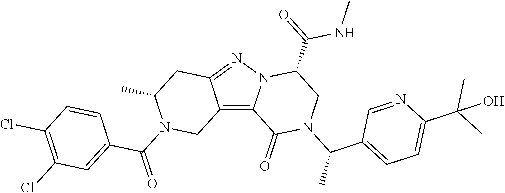 [SMILES: CNC(=O)[C@@H]1CN([C@@H](C)c2ccc(C(C)(C)O)nc2)C(=O)c2c3c(nn21)C[C@@H](C)N(C(=O)c1ccc(Cl)c(Cl)c1)C3]

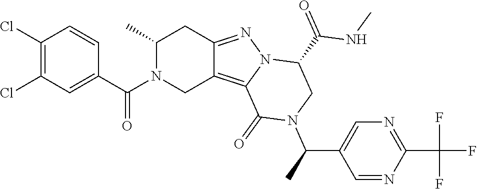 [SMILES: CNC(=O)[C@@H]1CN([C@H](C)c2cnc(C(F)(F)F)nc2)C(=O)c2c3c(nn21)C[C@@H](C)N(C(=O)c1ccc(Cl)c(Cl)c1)C3]

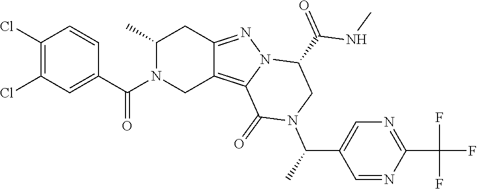 [SMILES: CNC(=O)[C@@H]1CN([C@@H](C)c2cnc(C(F)(F)F)nc2)C(=O)c2c3c(nn21)C[C@@H](C)N(C(=O)c1ccc(Cl)c(Cl)c1)C3]

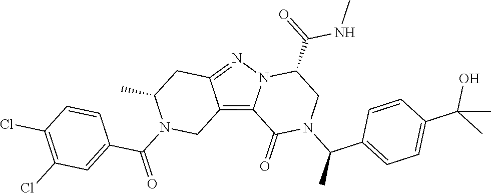 [SMILES: CNC(=O)[C@@H]1CN([C@H](C)c2ccc(C(C)(C)O)cc2)C(=O)c2c3c(nn21)C[C@@H](C)N(C(=O)c1ccc(Cl)c(Cl)c1)C3]

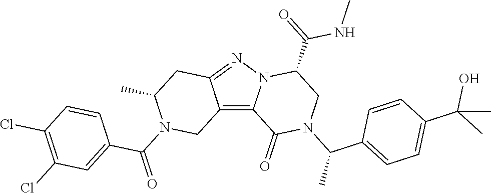 [SMILES: CNC(=O)[C@@H]1CN([C@@H](C)c2ccc(C(C)(C)O)cc2)C(=O)c2c3c(nn21)C[C@@H](C)N(C(=O)c1ccc(Cl)c(Cl)c1)C3]

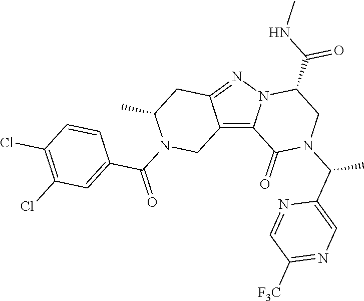 [SMILES: CNC(=O)[C@@H]1CN([C@H](C)c2cnc(C(F)(F)F)cn2)C(=O)c2c3c(nn21)C[C@@H](C)N(C(=O)c1ccc(Cl)c(Cl)c1)C3]

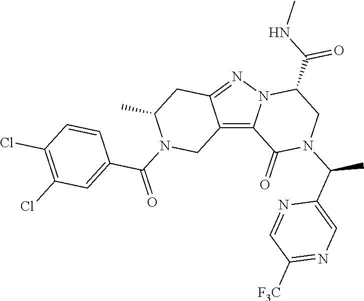 [SMILES: CNC(=O)[C@@H]1CN([C@@H](C)c2cnc(C(F)(F)F)cn2)C(=O)c2c3c(nn21)C[C@@H](C)N(C(=O)c1ccc(Cl)c(Cl)c1)C3]

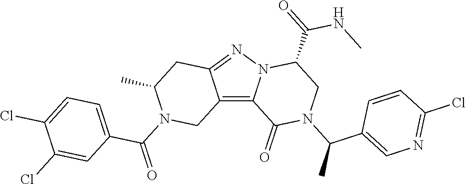 [SMILES: CNC(=O)[C@@H]1CN([C@H](C)c2ccc(Cl)nc2)C(=O)c2c3c(nn21)C[C@@H](C)N(C(=O)c1ccc(Cl)c(Cl)c1)C3]

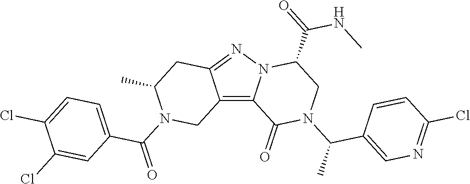 [SMILES: CNC(=O)[C@@H]1CN([C@@H](C)c2ccc(Cl)nc2)C(=O)c2c3c(nn21)C[C@@H](C)N(C(=O)c1ccc(Cl)c(Cl)c1)C3]

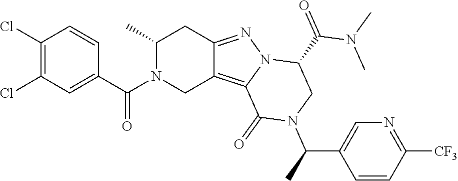 [SMILES: C[C@@H]1Cc2nn3c(c2CN1C(=O)c1ccc(Cl)c(Cl)c1)C(=O)N([C@H](C)c1ccc(C(F)(F)F)nc1)C[C@H]3C(=O)N(C)C]

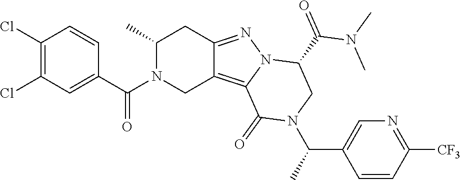 [SMILES: C[C@@H]1Cc2nn3c(c2CN1C(=O)c1ccc(Cl)c(Cl)c1)C(=O)N([C@@H](C)c1ccc(C(F)(F)F)nc1)C[C@H]3C(=O)N(C)C]

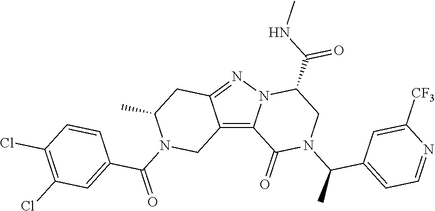 [SMILES: CNC(=O)[C@@H]1CN([C@H](C)c2ccnc(C(F)(F)F)c2)C(=O)c2c3c(nn21)C[C@@H](C)N(C(=O)c1ccc(Cl)c(Cl)c1)C3]

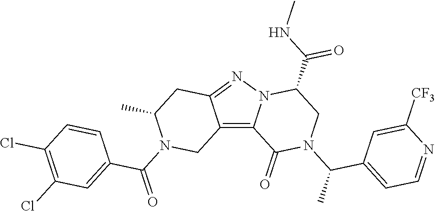 [SMILES: CNC(=O)[C@@H]1CN([C@@H](C)c2ccnc(C(F)(F)F)c2)C(=O)c2c3c(nn21)C[C@@H](C)N(C(=O)c1ccc(Cl)c(Cl)c1)C3]

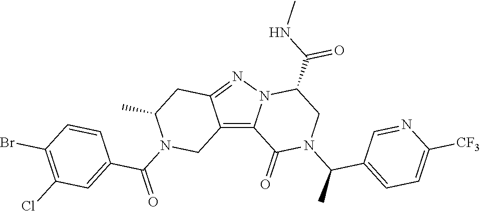 [SMILES: CNC(=O)[C@@H]1CN([C@H](C)c2ccc(C(F)(F)F)nc2)C(=O)c2c3c(nn21)C[C@@H](C)N(C(=O)c1ccc(Br)c(Cl)c1)C3]

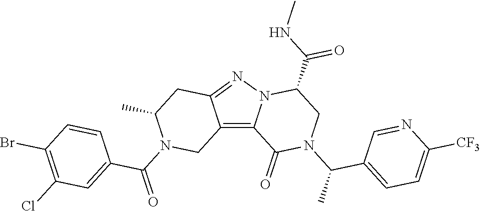 [SMILES: CNC(=O)[C@@H]1CN([C@@H](C)c2ccc(C(F)(F)F)nc2)C(=O)c2c3c(nn21)C[C@@H](C)N(C(=O)c1ccc(Br)c(Cl)c1)C3]

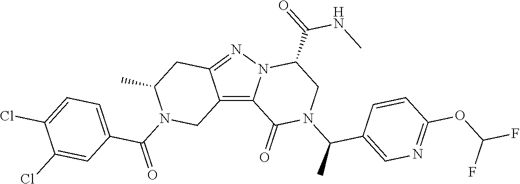 [SMILES: CNC(=O)[C@@H]1CN([C@H](C)c2ccc(OC(F)F)nc2)C(=O)c2c3c(nn21)C[C@@H](C)N(C(=O)c1ccc(Cl)c(Cl)c1)C3]

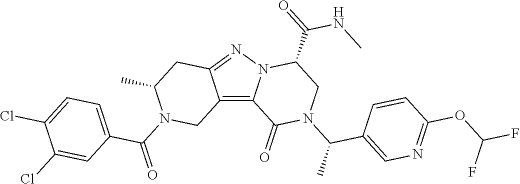 [SMILES: CNC(=O)[C@@H]1CN([C@@H](C)c2ccc(OC(F)F)nc2)C(=O)c2c3c(nn21)C[C@@H](C)N(C(=O)c1ccc(Cl)c(Cl)c1)C3]

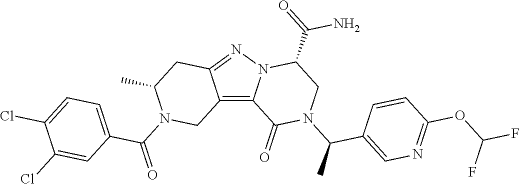 [SMILES: C[C@@H]1Cc2nn3c(c2CN1C(=O)c1ccc(Cl)c(Cl)c1)C(=O)N([C@H](C)c1ccc(OC(F)F)nc1)C[C@H]3C(N)=O]

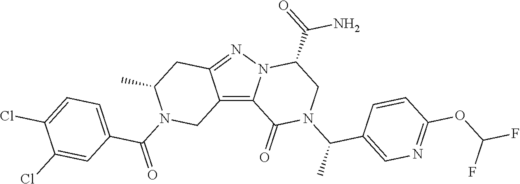 [SMILES: C[C@@H]1Cc2nn3c(c2CN1C(=O)c1ccc(Cl)c(Cl)c1)C(=O)N([C@@H](C)c1ccc(OC(F)F)nc1)C[C@H]3C(N)=O]

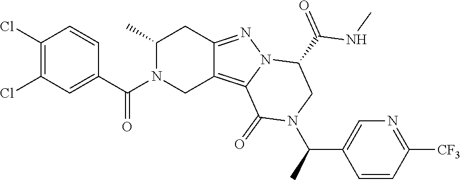 [SMILES: CNC(=O)[C@@H]1CN([C@H](C)c2ccc(C(F)(F)F)nc2)C(=O)c2c3c(nn21)C[C@@H](C)N(C(=O)c1ccc(Cl)c(Cl)c1)C3]

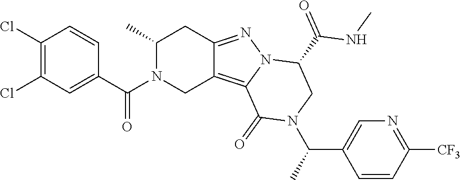 [SMILES: CNC(=O)[C@@H]1CN([C@@H](C)c2ccc(C(F)(F)F)nc2)C(=O)c2c3c(nn21)C[C@@H](C)N(C(=O)c1ccc(Cl)c(Cl)c1)C3]

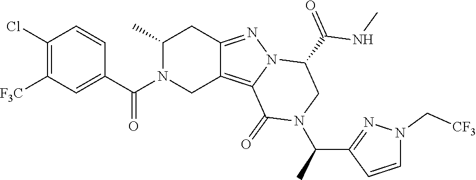 [SMILES: CNC(=O)[C@@H]1CN([C@H](C)c2ccn(CC(F)(F)F)n2)C(=O)c2c3c(nn21)C[C@@H](C)N(C(=O)c1ccc(Cl)c(C(F)(F)F)c1)C3]

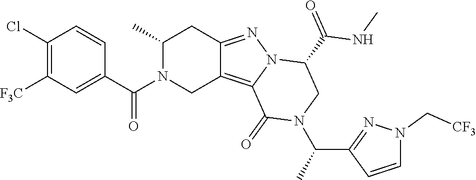 [SMILES: CNC(=O)[C@@H]1CN([C@@H](C)c2ccn(CC(F)(F)F)n2)C(=O)c2c3c(nn21)C[C@@H](C)N(C(=O)c1ccc(Cl)c(C(F)(F)F)c1)C3]

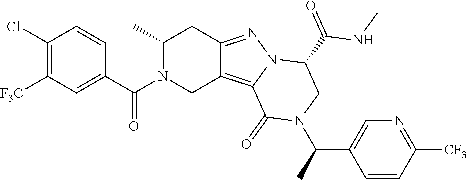 [SMILES: CNC(=O)[C@@H]1CN([C@H](C)c2ccc(C(F)(F)F)nc2)C(=O)c2c3c(nn21)C[C@@H](C)N(C(=O)c1ccc(Cl)c(C(F)(F)F)c1)C3]

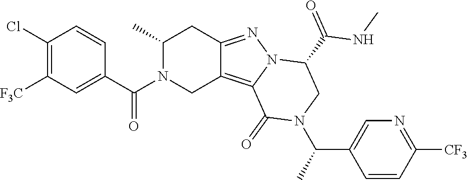 [SMILES: CNC(=O)[C@@H]1CN([C@@H](C)c2ccc(C(F)(F)F)nc2)C(=O)c2c3c(nn21)C[C@@H](C)N(C(=O)c1ccc(Cl)c(C(F)(F)F)c1)C3]

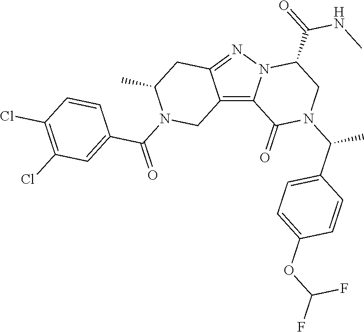 [SMILES: CNC(=O)[C@@H]1CN([C@H](C)c2ccc(OC(F)F)cc2)C(=O)c2c3c(nn21)C[C@@H](C)N(C(=O)c1ccc(Cl)c(Cl)c1)C3]

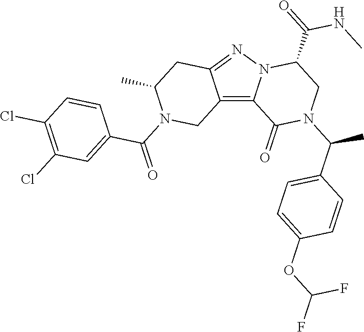 [SMILES: CNC(=O)[C@@H]1CN([C@@H](C)c2ccc(OC(F)F)cc2)C(=O)c2c3c(nn21)C[C@@H](C)N(C(=O)c1ccc(Cl)c(Cl)c1)C3]

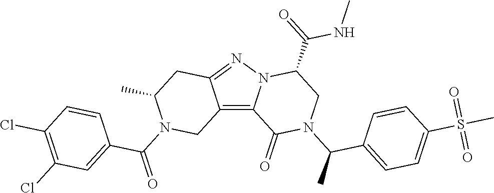 [SMILES: CNC(=O)[C@@H]1CN([C@H](C)c2ccc(S(C)(=O)=O)cc2)C(=O)c2c3c(nn21)C[C@@H](C)N(C(=O)c1ccc(Cl)c(Cl)c1)C3]

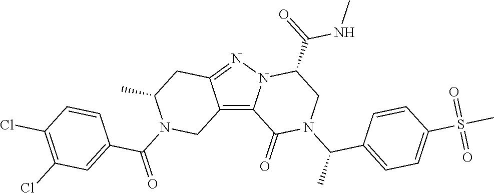 [SMILES: CNC(=O)[C@@H]1CN([C@@H](C)c2ccc(S(C)(=O)=O)cc2)C(=O)c2c3c(nn21)C[C@@H](C)N(C(=O)c1ccc(Cl)c(Cl)c1)C3]

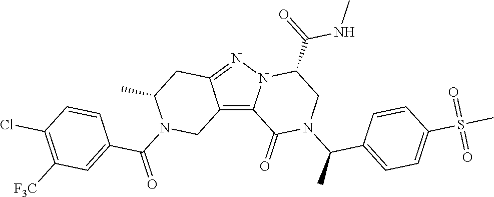 [SMILES: CNC(=O)[C@@H]1CN([C@H](C)c2ccc(S(C)(=O)=O)cc2)C(=O)c2c3c(nn21)C[C@@H](C)N(C(=O)c1ccc(Cl)c(C(F)(F)F)c1)C3]

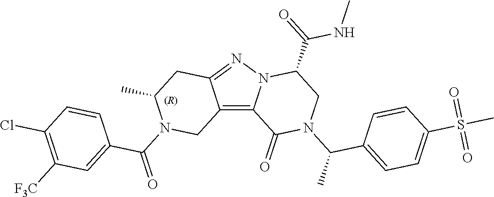

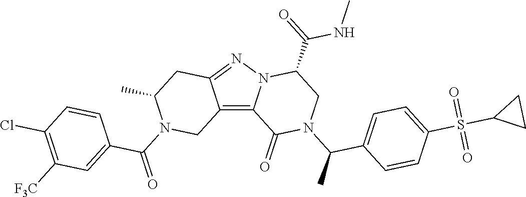 [SMILES: CNC(=O)[C@@H]1CN([C@H](C)c2ccc(S(=O)(=O)C3CC3)cc2)C(=O)c2c3c(nn21)C[C@@H](C)N(C(=O)c1ccc(Cl)c(C(F)(F)F)c1)C3]

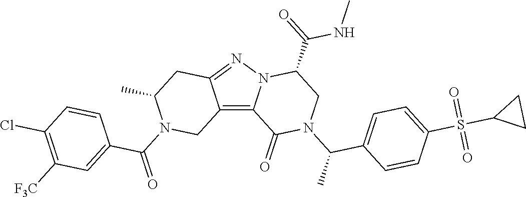 [SMILES: CNC(=O)[C@@H]1CN([C@@H](C)c2ccc(S(=O)(=O)C3CC3)cc2)C(=O)c2c3c(nn21)C[C@@H](C)N(C(=O)c1ccc(Cl)c(C(F)(F)F)c1)C3]

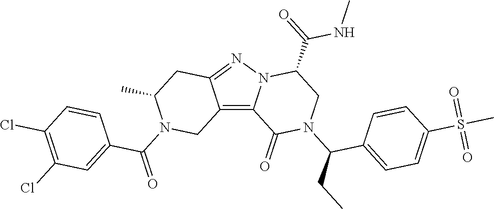 [SMILES: CC[C@H](c1ccc(S(C)(=O)=O)cc1)N1C[C@@H](C(=O)NC)n2nc3c(c2C1=O)CN(C(=O)c1ccc(Cl)c(Cl)c1)[C@H](C)C3]

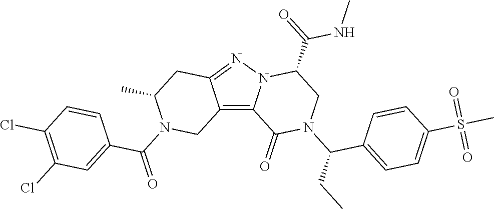 [SMILES: CC[C@@H](c1ccc(S(C)(=O)=O)cc1)N1C[C@@H](C(=O)NC)n2nc3c(c2C1=O)CN(C(=O)c1ccc(Cl)c(Cl)c1)[C@H](C)C3]

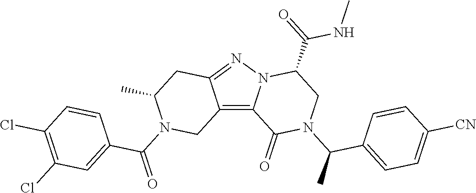 [SMILES: CNC(=O)[C@@H]1CN([C@H](C)c2ccc(C#N)cc2)C(=O)c2c3c(nn21)C[C@@H](C)N(C(=O)c1ccc(Cl)c(Cl)c1)C3]

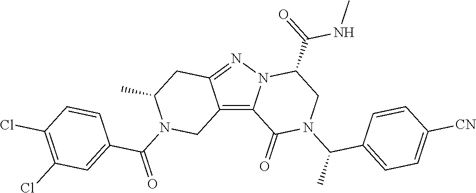 [SMILES: CNC(=O)[C@@H]1CN([C@@H](C)c2ccc(C#N)cc2)C(=O)c2c3c(nn21)C[C@@H](C)N(C(=O)c1ccc(Cl)c(Cl)c1)C3]

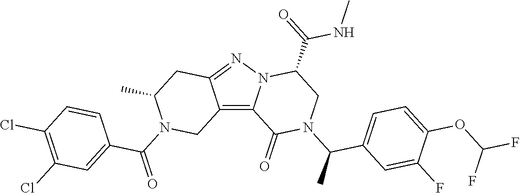 [SMILES: CNC(=O)[C@@H]1CN([C@H](C)c2ccc(OC(F)F)c(F)c2)C(=O)c2c3c(nn21)C[C@@H](C)N(C(=O)c1ccc(Cl)c(Cl)c1)C3]

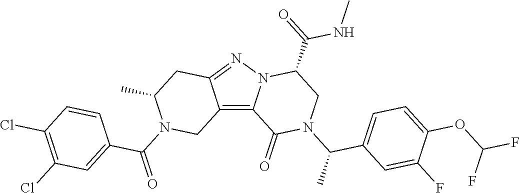 [SMILES: CNC(=O)[C@@H]1CN([C@@H](C)c2ccc(OC(F)F)c(F)c2)C(=O)c2c3c(nn21)C[C@@H](C)N(C(=O)c1ccc(Cl)c(Cl)c1)C3]

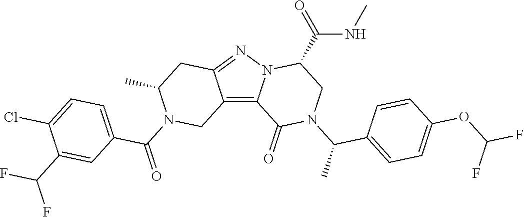 [SMILES: CNC(=O)[C@@H]1CN([C@@H](C)c2ccc(OC(F)F)cc2)C(=O)c2c3c(nn21)C[C@@H](C)N(C(=O)c1ccc(Cl)c(C(F)F)c1)C3]

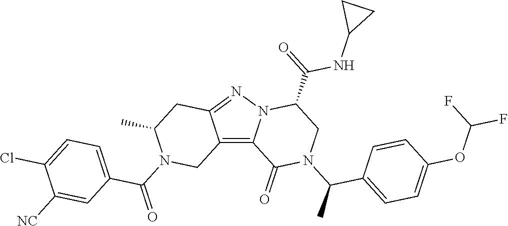 [SMILES: C[C@@H]1Cc2nn3c(c2CN1C(=O)c1ccc(Cl)c(C#N)c1)C(=O)N([C@H](C)c1ccc(OC(F)F)cc1)C[C@H]3C(=O)NC1CC1]

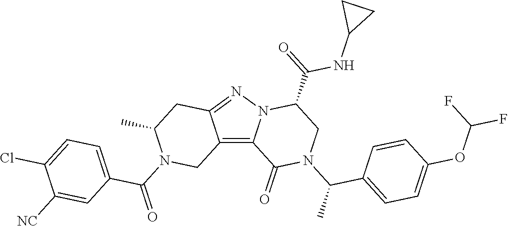 [SMILES: C[C@@H]1Cc2nn3c(c2CN1C(=O)c1ccc(Cl)c(C#N)c1)C(=O)N([C@@H](C)c1ccc(OC(F)F)cc1)C[C@H]3C(=O)NC1CC1]

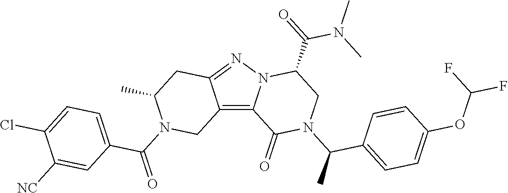 [SMILES: C[C@@H]1Cc2nn3c(c2CN1C(=O)c1ccc(Cl)c(C#N)c1)C(=O)N([C@H](C)c1ccc(OC(F)F)cc1)C[C@H]3C(=O)N(C)C]

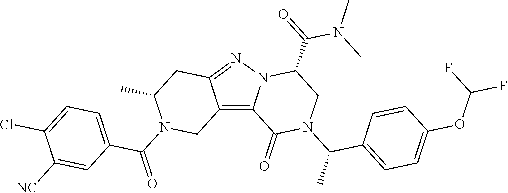 [SMILES: C[C@@H]1Cc2nn3c(c2CN1C(=O)c1ccc(Cl)c(C#N)c1)C(=O)N([C@@H](C)c1ccc(OC(F)F)cc1)C[C@H]3C(=O)N(C)C]

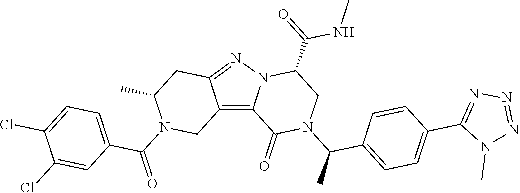 [SMILES: CNC(=O)[C@@H]1CN([C@H](C)c2ccc(-c3nnnn3C)cc2)C(=O)c2c3c(nn21)C[C@@H](C)N(C(=O)c1ccc(Cl)c(Cl)c1)C3]

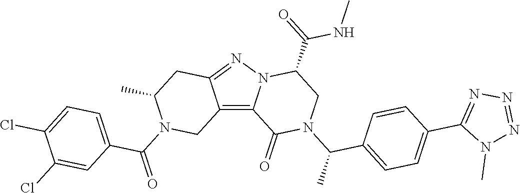 [SMILES: CNC(=O)[C@@H]1CN([C@@H](C)c2ccc(-c3nnnn3C)cc2)C(=O)c2c3c(nn21)C[C@@H](C)N(C(=O)c1ccc(Cl)c(Cl)c1)C3]

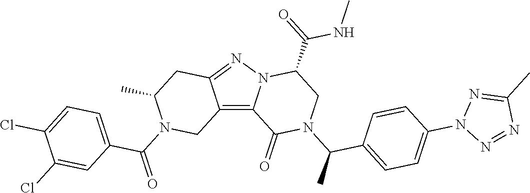 [SMILES: CNC(=O)[C@@H]1CN([C@H](C)c2ccc(-n3nnc(C)n3)cc2)C(=O)c2c3c(nn21)C[C@@H](C)N(C(=O)c1ccc(Cl)c(Cl)c1)C3]

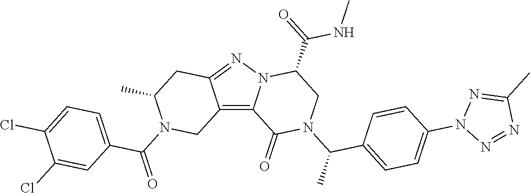 [SMILES: CNC(=O)[C@@H]1CN([C@@H](C)c2ccc(-n3nnc(C)n3)cc2)C(=O)c2c3c(nn21)C[C@@H](C)N(C(=O)c1ccc(Cl)c(Cl)c1)C3]

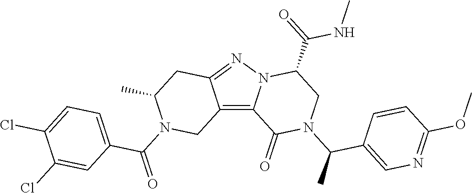 [SMILES: CNC(=O)[C@@H]1CN([C@H](C)c2ccc(OC)nc2)C(=O)c2c3c(nn21)C[C@@H](C)N(C(=O)c1ccc(Cl)c(Cl)c1)C3]

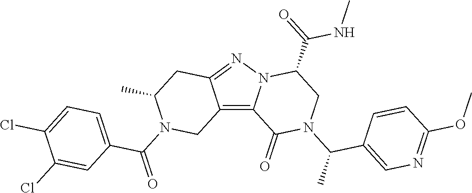 [SMILES: CNC(=O)[C@@H]1CN([C@@H](C)c2ccc(OC)nc2)C(=O)c2c3c(nn21)C[C@@H](C)N(C(=O)c1ccc(Cl)c(Cl)c1)C3]

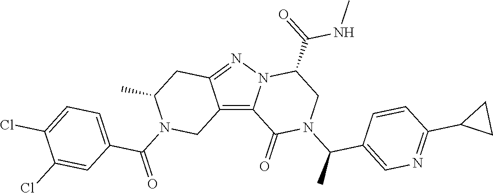 [SMILES: CNC(=O)[C@@H]1CN([C@H](C)c2ccc(C3CC3)nc2)C(=O)c2c3c(nn21)C[C@@H](C)N(C(=O)c1ccc(Cl)c(Cl)c1)C3]

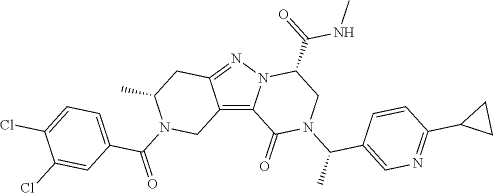 [SMILES: CNC(=O)[C@@H]1CN([C@@H](C)c2ccc(C3CC3)nc2)C(=O)c2c3c(nn21)C[C@@H](C)N(C(=O)c1ccc(Cl)c(Cl)c1)C3]

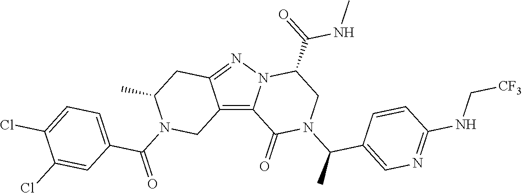 [SMILES: CNC(=O)[C@@H]1CN([C@H](C)c2ccc(NCC(F)(F)F)nc2)C(=O)c2c3c(nn21)C[C@@H](C)N(C(=O)c1ccc(Cl)c(Cl)c1)C3]

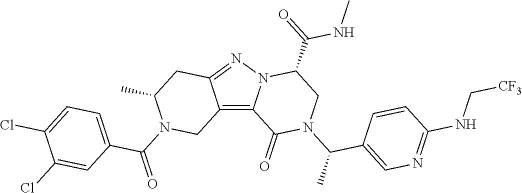 [SMILES: CNC(=O)[C@@H]1CN([C@@H](C)c2ccc(NCC(F)(F)F)nc2)C(=O)c2c3c(nn21)C[C@@H](C)N(C(=O)c1ccc(Cl)c(Cl)c1)C3]

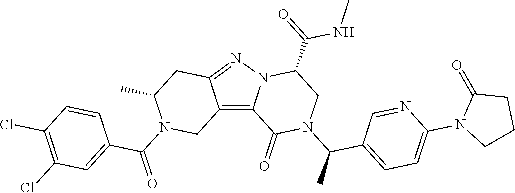 [SMILES: CNC(=O)[C@@H]1CN([C@H](C)c2ccc(N3CCCC3=O)nc2)C(=O)c2c3c(nn21)C[C@@H](C)N(C(=O)c1ccc(Cl)c(Cl)c1)C3]

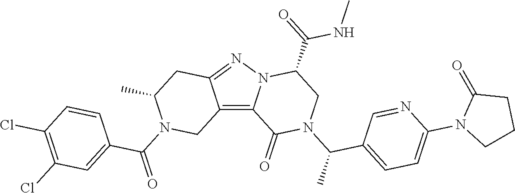 [SMILES: CNC(=O)[C@@H]1CN([C@@H](C)c2ccc(N3CCCC3=O)nc2)C(=O)c2c3c(nn21)C[C@@H](C)N(C(=O)c1ccc(Cl)c(Cl)c1)C3]

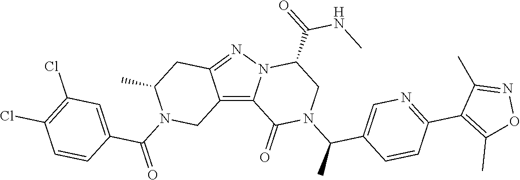 [SMILES: CNC(=O)[C@@H]1CN([C@H](C)c2ccc(-c3c(C)noc3C)nc2)C(=O)c2c3c(nn21)C[C@@H](C)N(C(=O)c1ccc(Cl)c(Cl)c1)C3]

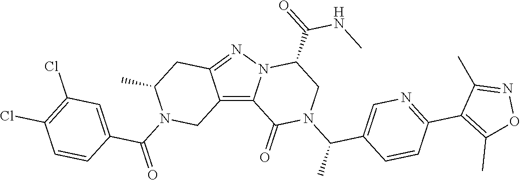 [SMILES: CNC(=O)[C@@H]1CN([C@@H](C)c2ccc(-c3c(C)noc3C)nc2)C(=O)c2c3c(nn21)C[C@@H](C)N(C(=O)c1ccc(Cl)c(Cl)c1)C3]

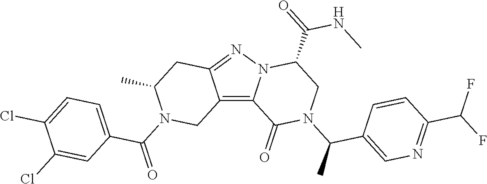 [SMILES: CNC(=O)[C@@H]1CN([C@H](C)c2ccc(C(F)F)nc2)C(=O)c2c3c(nn21)C[C@@H](C)N(C(=O)c1ccc(Cl)c(Cl)c1)C3]

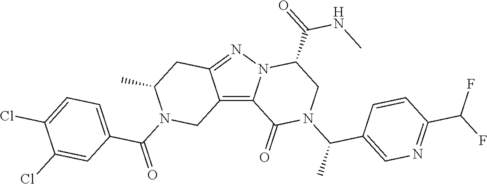 [SMILES: CNC(=O)[C@@H]1CN([C@@H](C)c2ccc(C(F)F)nc2)C(=O)c2c3c(nn21)C[C@@H](C)N(C(=O)c1ccc(Cl)c(Cl)c1)C3]

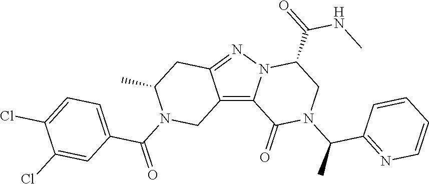 [SMILES: CNC(=O)[C@@H]1CN([C@H](C)c2ccccn2)C(=O)c2c3c(nn21)C[C@@H](C)N(C(=O)c1ccc(Cl)c(Cl)c1)C3]

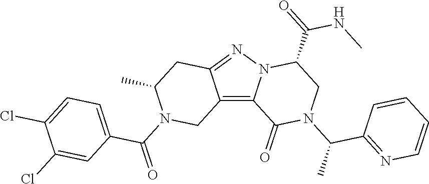 [SMILES: CNC(=O)[C@@H]1CN([C@@H](C)c2ccccn2)C(=O)c2c3c(nn21)C[C@@H](C)N(C(=O)c1ccc(Cl)c(Cl)c1)C3]

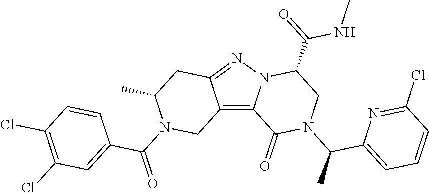 [SMILES: CNC(=O)[C@@H]1CN([C@H](C)c2cccc(Cl)n2)C(=O)c2c3c(nn21)C[C@@H](C)N(C(=O)c1ccc(Cl)c(Cl)c1)C3]

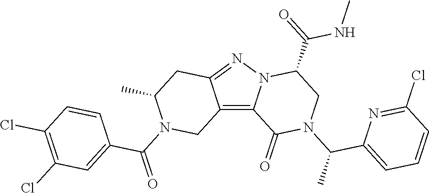 [SMILES: CNC(=O)[C@@H]1CN([C@@H](C)c2cccc(Cl)n2)C(=O)c2c3c(nn21)C[C@@H](C)N(C(=O)c1ccc(Cl)c(Cl)c1)C3]

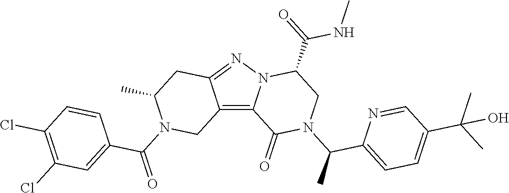 [SMILES: CNC(=O)[C@@H]1CN([C@H](C)c2ccc(C(C)(C)O)cn2)C(=O)c2c3c(nn21)C[C@@H](C)N(C(=O)c1ccc(Cl)c(Cl)c1)C3]

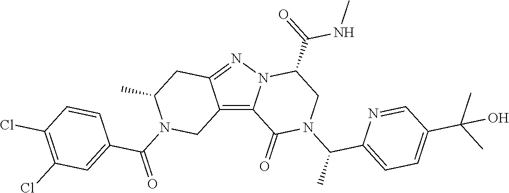 [SMILES: CNC(=O)[C@@H]1CN([C@@H](C)c2ccc(C(C)(C)O)cn2)C(=O)c2c3c(nn21)C[C@@H](C)N(C(=O)c1ccc(Cl)c(Cl)c1)C3]

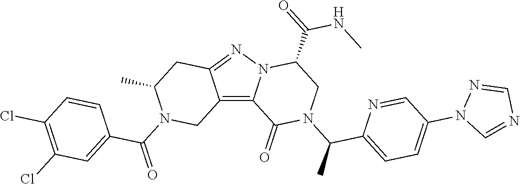 [SMILES: CNC(=O)[C@@H]1CN([C@H](C)c2ccc(-n3cncn3)cn2)C(=O)c2c3c(nn21)C[C@@H](C)N(C(=O)c1ccc(Cl)c(Cl)c1)C3]

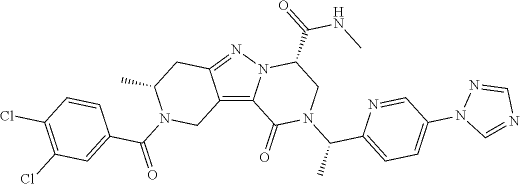 [SMILES: CNC(=O)[C@@H]1CN([C@@H](C)c2ccc(-n3cncn3)cn2)C(=O)c2c3c(nn21)C[C@@H](C)N(C(=O)c1ccc(Cl)c(Cl)c1)C3]

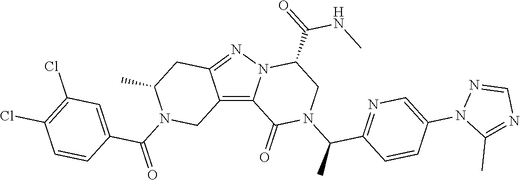 [SMILES: CNC(=O)[C@@H]1CN([C@H](C)c2ccc(-n3ncnc3C)cn2)C(=O)c2c3c(nn21)C[C@@H](C)N(C(=O)c1ccc(Cl)c(Cl)c1)C3]

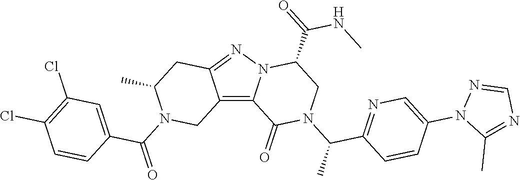 [SMILES: CNC(=O)[C@@H]1CN([C@@H](C)c2ccc(-n3ncnc3C)cn2)C(=O)c2c3c(nn21)C[C@@H](C)N(C(=O)c1ccc(Cl)c(Cl)c1)C3]

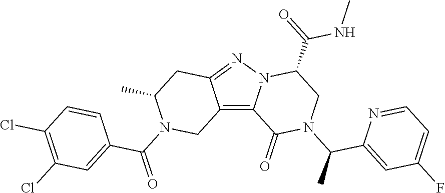 [SMILES: CNC(=O)[C@@H]1CN([C@H](C)c2cc(F)ccn2)C(=O)c2c3c(nn21)C[C@@H](C)N(C(=O)c1ccc(Cl)c(Cl)c1)C3]

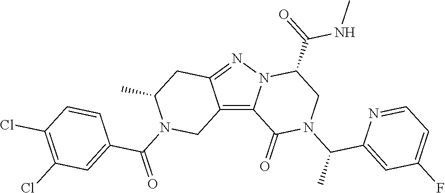 [SMILES: CNC(=O)[C@@H]1CN([C@@H](C)c2cc(F)ccn2)C(=O)c2c3c(nn21)C[C@@H](C)N(C(=O)c1ccc(Cl)c(Cl)c1)C3]

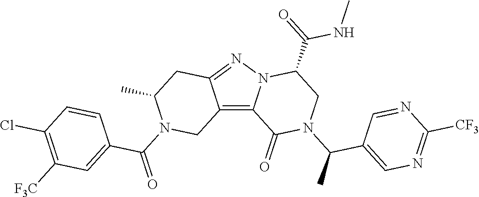 [SMILES: CNC(=O)[C@@H]1CN([C@H](C)c2cnc(C(F)(F)F)nc2)C(=O)c2c3c(nn21)C[C@@H](C)N(C(=O)c1ccc(Cl)c(C(F)(F)F)c1)C3]

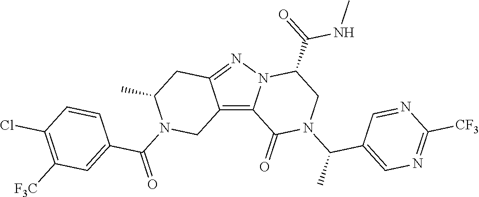 [SMILES: CNC(=O)[C@@H]1CN([C@@H](C)c2cnc(C(F)(F)F)nc2)C(=O)c2c3c(nn21)C[C@@H](C)N(C(=O)c1ccc(Cl)c(C(F)(F)F)c1)C3]

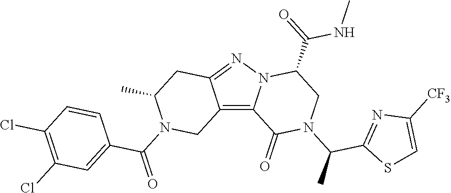 [SMILES: CNC(=O)[C@@H]1CN([C@H](C)c2nc(C(F)(F)F)cs2)C(=O)c2c3c(nn21)C[C@@H](C)N(C(=O)c1ccc(Cl)c(Cl)c1)C3]

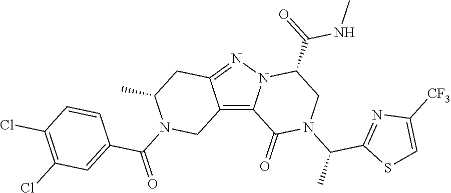 [SMILES: CNC(=O)[C@@H]1CN([C@@H](C)c2nc(C(F)(F)F)cs2)C(=O)c2c3c(nn21)C[C@@H](C)N(C(=O)c1ccc(Cl)c(Cl)c1)C3]

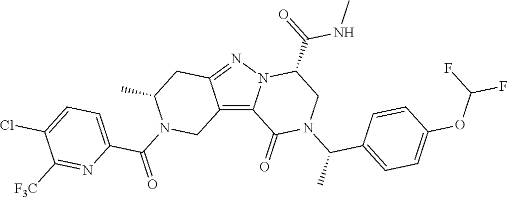 [SMILES: CNC(=O)[C@@H]1CN([C@@H](C)c2ccc(OC(F)F)cc2)C(=O)c2c3c(nn21)C[C@@H](C)N(C(=O)c1ccc(Cl)c(C(F)(F)F)n1)C3]

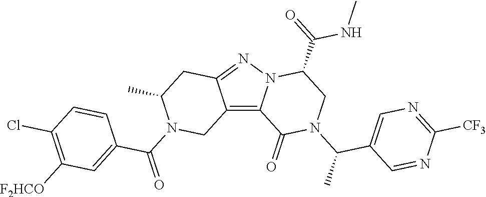 [SMILES: CNC(=O)[C@@H]1CN([C@@H](C)c2cnc(C(F)(F)F)nc2)C(=O)c2c3c(nn21)C[C@@H](C)N(C(=O)c1ccc(Cl)c(OC(F)F)c1)C3]

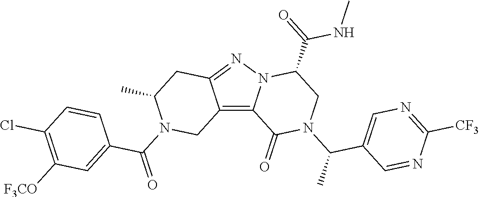 [SMILES: CNC(=O)[C@@H]1CN([C@@H](C)c2cnc(C(F)(F)F)nc2)C(=O)c2c3c(nn21)C[C@@H](C)N(C(=O)c1ccc(Cl)c(OC(F)(F)F)c1)C3]

### Pharmaceutical Compositions

Also disclosed herein are pharmaceutical compositions comprising
(A) at least one compound of any one of Formula (I), in any one of the embodiments defined above, or a pharmaceutically acceptable salt thereof, and
(B) at least one pharmaceutically acceptable excipient.

In embodiments, the pharmaceutical composition comprises at least one additional active or therapeutic agent. Additional active therapeutic agents may include, for example, an anti-HBV agent such as an HBV polymerase inhibitor, interferon, viral entry inhibitor, viral maturation inhibitor, capsid assembly modulator, reverse transcriptase inhibitor, immunomodulatory agent such as a TLR-agonist, or any other agents that affect the HBV life cycle and/or the consequences of HBV infection. The active agents of the present disclosure are used, alone or in combination with one or more additional active agents, to formulate pharmaceutical compositions of the present disclosure.

As used herein, the term “composition” or “pharmaceutical composition” refers to a mixture of at least one compound useful within the present disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the present disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the present disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure and are physiologically acceptable to the patient. Supplementary active compounds may also be incorporated into the compositions. Other additional ingredients that may be included in the pharmaceutical compositions used in the practice of the present disclosure are known in the art and described, for example in Remington's Pharmaceutical Sciences (Genaro, Ed., Mack Publishing Co., 1985, Easton, PA), which is incorporated herein by reference.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the present disclosure can be provided in the form of tablets or capsules, or as a solution, emulsion, or suspension. To prepare the oral compositions, the compounds may be formulated to yield a dosage of, e.g., from about 0.05 to about 100 mg/kg daily, or from about 0.05 to about 35 mg/kg daily, or from about 0.1 to about 10 mg/kg daily. For example, a total daily dosage of about 5 mg to 5 g daily may be accomplished by dosing once, twice, three, or four times per day.

Oral tablets may include a compound according to the present disclosure mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract or may be coated with an enteric coating.

Capsules for oral administration include hard and soft gelatin capsules. To prepare hard gelatin capsules, compounds of the present disclosure may be mixed with a solid, semi-solid, or liquid diluent. Soft gelatin capsules may be prepared by mixing the compound of the present disclosure with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this present disclosure may also be administered by non-oral routes. For example, the compositions may be formulated for rectal administration as a suppository. For parenteral use, including intravenous, intramuscular, intraperitoneal, or subcutaneous routes, the compounds of the present disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compounds of the present disclosure may utilize a patch formulation to affect transdermal delivery.

Compounds of the present disclosure may alternatively be administered in methods of this present disclosure by inhalation, via the nasal or oral routes, e.g., in a spray formulation also containing a suitable carrier.

### Methods of Use

The disclosed compounds are useful in the prevention or treatment of an HBV infection or of an HBV-induced disease in mammal in need thereof, more particularly in a human in need thereof.

In a non-limiting aspect, these compounds may (i) modulate or disrupt HBV assembly and other HBV core protein functions necessary for HBV replication or the generation of infectious particles, (ii) inhibit the production of infectious virus particles or infection, or (iii) interact with HBV capsid to effect defective viral particles with reduced infectivity or replication capacity acting as capsid assembly modulators. In particular, and without being bound to any particular mechanism of action, it is believed that the disclosed compounds are useful in HBV treatment by disrupting, accelerating, reducing, delaying and/or inhibiting normal viral capsid assembly and/or disassembly of immature or mature particles, thereby inducing aberrant capsid morphology leading to antiviral effects such as disruption of virion assembly and/or disassembly, virion maturation, virus egress and/or infection of target cells. The disclosed compounds may act as a disruptor of capsid assembly interacting with mature or immature viral capsid to perturb the stability of the capsid, thus affecting its assembly and/or disassembly. The disclosed compounds may perturb protein folding and/or salt bridges required for stability, function and/or normal morphology of the viral capsid, thereby disrupting and/or accelerating capsid assembly and/or disassembly. The disclosed compounds may bind capsid and alter metabolism of cellular polyproteins and precursors, leading to abnormal accumulation of protein monomers and/or oligomers and/or abnormal particles, which causes cellular toxicity and death of infected cells. The disclosed compounds may cause failure of the formation of capsids of optimal stability, affecting efficient uncoating and/or disassembly of viruses (e.g., during infectivity). The disclosed compounds may disrupt and/or accelerate capsid assembly and/or disassembly when the capsid protein is immature. The disclosed compounds may disrupt and/or accelerate capsid assembly and/or disassembly when the capsid protein is mature. The disclosed compounds may disrupt and/or accelerate capsid assembly and/or disassembly during viral infectivity which may further attenuate HBV viral infectivity and/or reduce viral load. The disruption, acceleration, inhibition, delay and/or reduction of capsid assembly and/or disassembly by the disclosed compounds may eradicate the virus from the host organism. Eradication of HBV from a subject by the disclosed compounds advantageously obviates the need for chronic long-term therapy and/or reduces the duration of long-term therapy.

An additional embodiment of the present disclosure is a method of treating a subject suffering from an HBV infection, comprising administering to a subject in need of such treatment an effective amount of at least one compound of Formula (I).

In another aspect, provided herein is a method of reducing the viral load associated with an HBV infection in an individual in need thereof, comprising administering to the individual a therapeutically effective amount of a compound of Formula (I), or a pharmaceutically acceptable salt thereof.

In another aspect, provided herein is a method of reducing reoccurrence of an HBV infection in an individual in need thereof, comprising administering to the individual a therapeutically effective amount of a compound of Formula (I), or a pharmaceutically acceptable salt thereof.

Additionally, HBV acts as a helper virus to hepatitis delta virus (HDV), and it is estimated that more than 15 million people may be HBV/HDV co-infected worldwide, with an increased risk of rapid progression to cirrhosis and increased hepatic decompensation, than patients suffering from HBV alone (Hughes, S. A. et al. Lancet 2011, 378, 73-85). HDV, infects therefore subjects suffering from HBV infection. In a particular embodiment, the compounds of the present disclosure may be used in the treatment and/or prophylaxis of HBV/HDV co-infection, or diseases associated with HBV/HDV co infection. Therefore, in a particular embodiment, the HBV infection is in particular HBV/HDV co-infection, and the mammal, in particular the human, may be HBV/HDV co-infected, or be at risk of HBV/HDV co infection.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula (I), or a pharmaceutically acceptable salt thereof.

In another aspect, provided herein is a method of reducing an adverse physiological impact of an HBV infection in an individual in need thereof, comprising administering to the individual a therapeutically effective amount of a compound of Formula (I), or a pharmaceutically acceptable salt thereof.

In another aspect, provided herein is a method of inducing remission of hepatic injury from an HBV infection in an individual in need thereof, comprising administering to the individual a therapeutically effective amount of a compound of Formula (I), or a pharmaceutically acceptable salt thereof.

In another aspect, provided herein is a method of reducing the physiological impact of long-term antiviral therapy for HBV infection in an individual in need thereof, comprising administering to the individual a therapeutically effective amount of a compound of Formula (I), or a pharmaceutically acceptable salt thereof.

In another aspect, provided herein is a method of prophylactically treating an HBV infection in an individual in need thereof, wherein the individual is afflicted with a latent HBV infection, comprising administering to the individual a therapeutically effective amount of a compound of Formula (I), or a pharmaceutically acceptable salt thereof.

In embodiments, the disclosed compounds are suitable for monotherapy. In embodiments, the disclosed compounds are effective against natural or native HBV strains.

In embodiments, the disclosed compounds are effective against HBV strains resistant to currently known drugs.

In another embodiment, the compounds provided herein can be used in methods of modulating (e.g., inhibiting or disrupting) the activity, stability, function, and viral replication properties of HBV cccDNA.

In yet another embodiment, the compounds of the present disclosure can be used in methods of diminishing or preventing the formation of HBV cccDNA.

In another embodiment, the compounds provided herein can be used in methods of modulating (e.g., inhibiting or disrupting) the activity of HBV cccDNA.

In yet another embodiment, the compounds of the present disclosure can be used in methods of diminishing the formation of HBV cccDNA.

In another embodiment, the disclosed compounds can be used in methods of modulating, inhibiting, or disrupting the generation or release of HBV RNA particles from within the infected cell.

In a further embodiment, the total burden (or concentration) of HBV RNA particles is modulated. In a preferred embodiment, the total burden of HBV RNA is diminished.

In another embodiment, the methods provided herein reduce the viral load in the individual to a greater extent or at a faster rate compared to the administering of a compound selected from the group consisting of an HBV polymerase inhibitor, interferon, viral entry inhibitor, viral maturation inhibitor, distinct capsid assembly modulator, antiviral compounds of distinct or unknown mechanism, and any combination thereof.

In another embodiment, the methods provided herein cause a lower incidence of viral mutation and/or viral resistance than the administering of a compound selected from the group consisting of an HBV polymerase inhibitor, interferon, viral entry inhibitor, viral maturation inhibitor, distinct capsid assembly modulator, antiviral compounds of distinct or unknown mechanism, and combination thereof.

In another embodiment, the methods provided herein further comprise administering to the individual at least one HBV vaccine, a nucleoside HBV inhibitor, an interferon or any combination thereof.

In an aspect, provided herein is a method of treating an HBV infection in an individual in need thereof, comprising reducing the HBV viral load by administering to the individual a therapeutically effective amount of a compound of Formula (I), or a pharmaceutically acceptable salt thereof, alone or in combination with a reverse transcriptase inhibitor; and further administering to the individual a therapeutically effective amount of HBV vaccine.

An additional embodiment of the present disclosure is a method of treating a subject suffering from an HBV infection, comprising administering to a subject in need of such treatment an effective amount of at least one compound of Formula (I).

In another aspect, provided herein is a method of reducing the viral load associated with an HBV infection in an individual in need thereof, comprising administering to the individual a therapeutically effective amount of a compound of Formula (I), or a pharmaceutically acceptable salt thereof.

In another aspect, provided herein is a method of reducing reoccurrence of an HBV infection in an individual in need thereof, comprising administering to the individual a therapeutically effective amount of a compound of Formula (I), or a pharmaceutically acceptable salt thereof.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula (I), or a pharmaceutically acceptable salt thereof.

In another aspect, provided herein is a method of reducing an adverse physiological impact of an HBV infection in an individual in need thereof, comprising administering to the individual a therapeutically effective amount of a compound of Formula (I), or a pharmaceutically acceptable salt thereof.

In another aspect, provided herein is a method of inducing remission of hepatic injury from an HBV infection in an individual in need thereof, comprising administering to the individual a therapeutically effective amount of a compound of Formula (I), or a pharmaceutically acceptable salt thereof.

In another aspect, provided herein is a method of reducing the physiological impact of long-term antiviral therapy for HBV infection in an individual in need thereof, comprising administering to the individual a therapeutically effective amount of a compound of Formula (I), or a pharmaceutically acceptable salt thereof.

In another aspect, provided herein is a method of prophylactically treating an HBV infection in an individual in need thereof, wherein the individual is afflicted with a latent HBV infection, comprising administering to the individual a therapeutically effective amount of a compound of Formula (I), or a pharmaceutically acceptable salt thereof.

In an embodiment, the methods provided herein further comprise monitoring the HBV viral load of the subject, wherein the method is carried out for a period of time such that the HBV virus is undetectable.

### Combinations

Provided herein are combinations of one or more of the disclosed compounds with at least one additional therapeutic agent. In embodiments, the methods provided herein can further comprise administering to the individual at least one additional therapeutic agent. In embodiments, the disclosed compounds are suitable for use in combination therapy. The compounds of the present disclosure may be useful in combination with one or more additional compounds useful for treating HBV infection. These additional compounds may comprise compounds of the present disclosure or compounds known to treat, prevent, or reduce the symptoms or effects of HBV infection.

In an exemplary embodiment, additional active ingredients are those that are known or discovered to be effective in the treatment of conditions or disorders involved in HBV infection, such as another HBV capsid assembly modulator or a compound active against another target associated with the particular condition or disorder involved in HBV infection, or the HBV infection itself. The combination may serve to increase efficacy (e.g., by including in the combination a compound potentiating the potency or effectiveness of an active agent according to the present disclosure), decrease one or more side effects, or decrease the required dose of the active agent according to the present disclosure. In a further embodiment, the methods provided herein allow for administering of the at least one additional therapeutic agent at a lower dose or frequency as compared to the administering of the at least one additional therapeutic agent alone that is required to achieve similar results in prophylactically treating an HBV infection in an individual in need thereof.

Such compounds include but are not limited to HBV combination drugs, HBV vaccines, HBV DNA polymerase inhibitors, immunomodulatory agents, toll-like receptor (TLR) modulators, interferon alpha receptor ligands, hyaluronidase inhibitors, hepatitis b surface antigen (HBsAg) inhibitors, cytotoxic T-lymphocyte-associated protein 4 (CTLA-4) inhibitors, cyclophilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, famesoid X receptor agonists, HBV antibodies, CCR2 chemokine antagonists, thymosin agonists, cytokines, nucleoprotein modulators, retinoic acid-inducible gene 1 simulators, NOD2 stimulators, phosphatidylinositol 3-kinase (PI3K) inhibitors, indoleamine-2, 3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and any other agent that affects the HBV life cycle and/or affect the consequences of HBV infection or combinations thereof.

In embodiments, the compounds of the present disclosure may be used in combination with an HBV polymerase inhibitor, immunomodulatory agents, interferon such as pegylated interferon, viral entry inhibitor, viral maturation inhibitor, capsid assembly modulator, reverse transcriptase inhibitor, a cyclophilin/TNF inhibitor, immunomodulatory agent such as a TLR-agonist, an HBV vaccine, and any other agent that affects the HBV life cycle and/or affect the consequences of HBV infection or combinations thereof. In particular, the compounds of the present disclosure may be used in combination with one or more agents (or a salt thereof) selected from the group consisting of
HBV reverse transcriptase inhibitors, and DNA and RNA polymerase inhibitors, including but not limited to: lamivudine (3TC, Zeffix, Heptovir, Epivir, and Epivir-HBV), entecavir (Baraclude, Entavir), adefovir dipivoxil (Hepsara, Preveon, bis-POM PMEA), tenofovir disoproxil fumarate (Viread, TDF or PMPA);
interferons, including but not limited to interferon alpha (IFN-α), interferon beta (IFN-β), interferon lambda (IFN-λ), and interferon gamma (IFN-γ);
viral entry inhibitors;
viral maturation inhibitors;
literature-described capsid assembly modulators, such as, but not limited to BAY 41-4109;
reverse transcriptase inhibitor;
an immunomodulatory agent such as a TLR-agonist; and
agents of distinct or unknown mechanism, such as but not limited to AT-61 ((E)-N-(1-chloro-3-oxo-1-phenyl-3-(piperidin-1-yl)prop-1-en-2-yl)benzamide), AT-130 ((E)-N-(1-bromo-1-(2-methoxyphenyl)-3-oxo-3-(piperidin-1-yl)prop-1-en-2-yl)-4-nitrobenzamide), and similar analogs.

In embodiments, the additional therapeutic agent is an interferon. The term “interferon” or “IFN” refers to any member the family of highly homologous species-specific proteins that inhibit viral replication and cellular proliferation and modulate immune response. Human interferons are grouped into three classes; Type I, which include interferon-alpha (IFN-α), interferon-beta (IFN-β), and interferon-omega (IFN-ω), Type II, which includes interferon-gamma (IFN-γ), and Type III, which includes interferon-lambda (IFN-λ). Recombinant forms of interferons that have been developed and are commercially available are encompassed by the term “interferon” as used herein. Subtypes of interferons, such as chemically modified or mutated interferons, are also encompassed by the term “interferon” as used herein. Chemically modified interferons include pegylated interferons and glycosylated interferons. Examples of interferons also include, but are not limited to, interferon-alpha-2a, interferon-alpha-2b, interferon-alpha-n1, interferon-beta-1a, interferon-beta-1b, interferon-lamda-1, interferon-lamda-2, and interferon-lamda-3. Examples of pegylated interferons include pegylated interferon-alpha-2a and pegylated interferon alpha-2b.

Accordingly, in one embodiment, the compounds of Formula I, can be administered in combination with an interferon selected from the group consisting of interferon alpha (IFN-α), interferon beta (IFN-β), interferon lambda (IFN-λ), and interferon gamma (IFN-γ). In one specific embodiment, the interferon is interferon-alpha-2a, interferon-alpha-2b, or interferon-alpha-n1. In another specific embodiment, the interferon-alpha-2a or interferon-alpha-2b is pegylated. In a preferred embodiment, the interferon-alpha-2a is pegylated interferon-alpha-2a (PEGASYS).

In another embodiment, the additional therapeutic agent is selected from immune modulator or immune stimulator therapies, which includes biological agents belonging to the interferon class.

Further, the additional therapeutic agent may be an agent that disrupts the function of other essential viral protein(s) or host proteins required for HBV replication or persistence.

In another embodiment, the additional therapeutic agent is an antiviral agent that blocks viral entry or maturation or targets the HBV polymerase such as nucleoside or nucleotide or non-nucleos(t)ide polymerase inhibitors. In a further embodiment of the combination therapy, the reverse transcriptase inhibitor and/or DNA and/or RNA polymerase inhibitor is Zidovudine, Didanosine, Zalcitabine, ddA, Stavudine, Lamivudine, Abacavir, Emtricitabine, Entecavir, Apricitabine, Atevirapine, ribavirin, acyclovir, famciclovir, valacyclovir, ganciclovir, valganciclovir, Tenofovir, Adefovir, PMPA, cidofovir, Efavirenz, Nevirapine, Delavirdine, or Etravirine.

In an embodiment, the additional therapeutic agent is an immunomodulatory agent that induces a natural, limited immune response leading to induction of immune responses against unrelated viruses. In other words, the immunomodulatory agent can affect maturation of antigen presenting cells, proliferation of T-cells and cytokine release (e.g., IL-12, IL-18, IFN-alpha, -beta, and -gamma and TNF-alpha among others).

In a further embodiment, the additional therapeutic agent is a TLR modulator or a TLR agonist, such as a TLR-7 agonist or TLR-9 agonist. In further embodiment of the combination therapy, the TLR-7 agonist is selected from the group consisting of SM360320 (9-benzyl-8-hydroxy-2-(2-methoxy-ethoxy)adenine) and AZD 8848 (methyl [3-({[3-(6-amino-2-butoxy-8-oxo-7,8-dihydro-9H-purin-9-yl)propyl][3-(4-morpholinyl)propyl]amino}methyl)phenyl]acetate).

In any of the methods provided herein, the method may further comprise administering to the individual at least one HBV vaccine, a nucleoside HBV inhibitor, an interferon or any combination thereof. In an embodiment, the HBV vaccine is at least one of RECOMBIVAX HB, ENGERIX-B, ELOVAC B, GENEVAC-B, or SHANVAC B.

In another aspect, provided herein is method of treating an HBV infection in an individual in need thereof, comprising reducing the HBV viral load by administering to the individual a therapeutically effective amount of a compound of the present disclosure alone or in combination with a reverse transcriptase inhibitor; and further administering to the individual a therapeutically effective amount of HBV vaccine. The reverse transcriptase inhibitor may be one of Zidovudine, Didanosine, Zalcitabine, ddA, Stavudine, Lamivudine, Abacavir, Emtricitabine, Entecavir, Apricitabine, Atevirapine, ribavirin, acyclovir, famciclovir, valacyclovir, ganciclovir, valganciclovir, Tenofovir, Adefovir, PMPA, cidofovir, Efavirenz, Nevirapine, Delavirdine, or Etravirine.

For any combination therapy described herein, synergistic effect may be calculated, for example, using suitable methods such as the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Beneficial Effects

The present compounds have improved human liver microsome stability as well as reasonable anti-HBV activity. As compared to comparative compounds, the half-life (t½) of the present compounds are significantly increased, showing great improvement in human metabolic stability and the advantages in pharmaceutical applications.

### Methods

The present disclosure relates to a method for the preparation of a compound of Formula (I) as described herein.

In an exemplary embodiment, the method comprises the steps of:
1) reacting a compound of Formula (a)

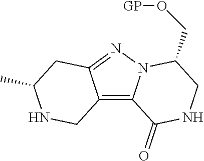

with a compound of Formula (b)

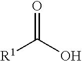

(wherein PG is a protecting group) to give a compound of Formula (c)

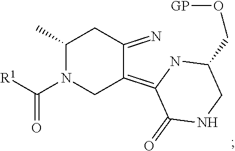

2) reacting the compound of Formula (c) with a compound of Formula (d)

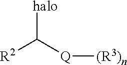

to give a compound of Formula (e)

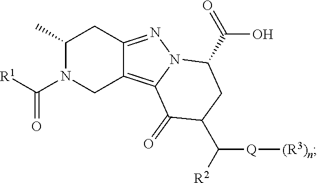

3) reacting the compound of Formula (e) with a compound of Formula (f)

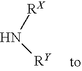

give a compound of Formula (I).
wherein R¹, R², R³, Q, halo, n, R^{x} and R^{y} are as defined herein.

In an embodiment, the compound of Formula (I) may be subjected to chiral separation to give individual enantiopure compound.

In an embodiment, in step 2), the reaction product of the compound of Formula (c) and the compound of Formula (d) is subjected to deprotection and oxidation to give the compound of Formula (e).

PG is a protecting group, which is conventionally used and is preferably TBDPS.

An exemplary scheme is as follows.

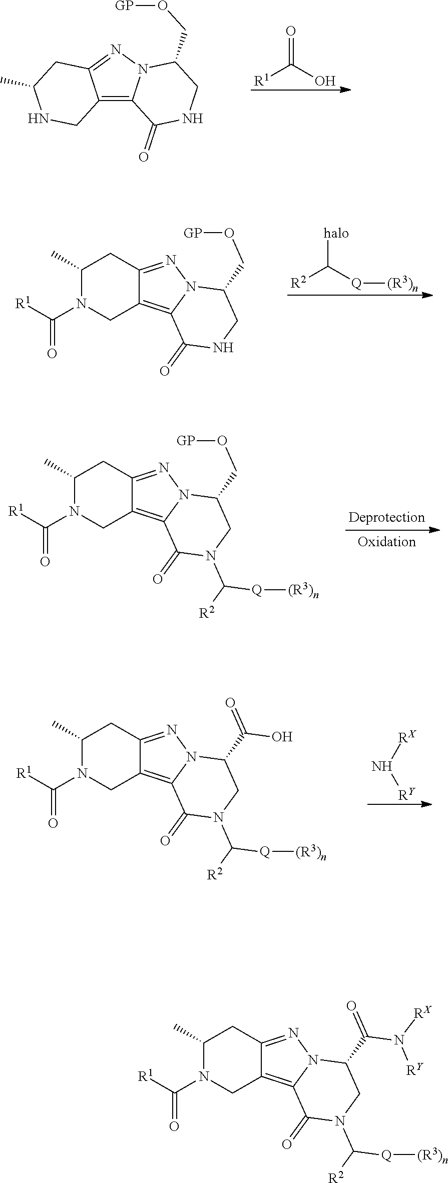

The present disclosure also relates to a method for the preparation of a compound of Formula (I) as described herein with a specific chirality like

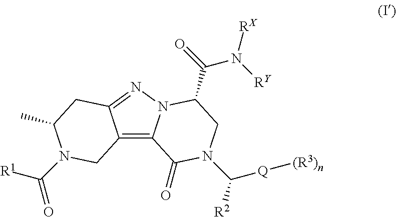

In an exemplary embodiment, the method comprises the steps of.
1) reacting a compound of Formula (g)

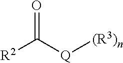

with a compound of

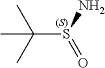

to give a compound of Formula (h)

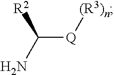

2) reacting the compound of Formula (h) with a compound of

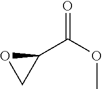

to give a compound of Formula (i)

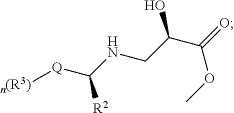

3) reacting the compound of Formula (i) with a compound of Formula (f)

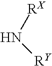

to give a compound of Formula (j)

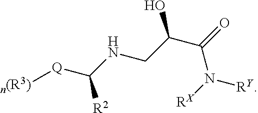

4) reacting the compound of Formula (j) with a compound of

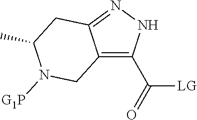

(wherein PG₁ is a protecting group, LG is a leaving group) to give a compound of Formula (k)

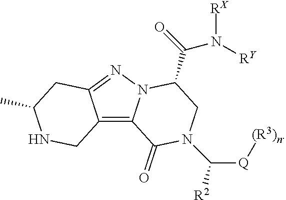

5) reacting the compound of Formula (k) with a compound of Formula (b)

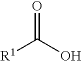

to give the compound of Formula (I′).
wherein R¹, R², R³, Q, n, R^{x} and R^{y} are as defined herein.

In an embodiment, in step 1), the reaction product of the compound of Formula (g) and the compound of

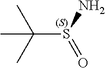

is treated with HCl to give Formula (h).

In an embodiment, in step 3), the reaction product Formula (j) may be further subjected to a step of reacting with a protecting group to give a compound of Formula (j′)

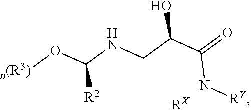

which is used instead of Formula U) step 4).

In an embodiment, in step 4), the reaction product of reacting the compound of Formula (j) with a compound of

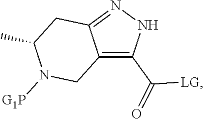

is subjected to ring closing reaction and deprotection to give the compound of Formula (k).

In an embodiment, in step 4), the compound of

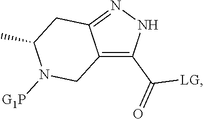

may further comprise a protecting group, as a compound of

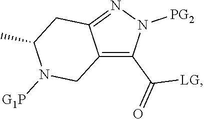

which is used instead of

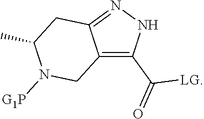

In an embodiment, in step 4) to step 5), the compound of Formula (b)

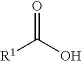

may react with the product of the compound of

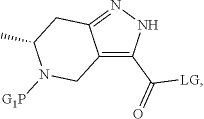

subjected to deprotection, and then with the compound of Formula (j), and the product is subjected to ring closing reaction to give the compound of formula (I′).

PG₁ and PG₂ are each protecting groups, which are conventionally used and may be preferably Boc or

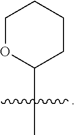 [SMILES: *C1CCCCO1]

LG is a leaving group, which is conventionally used and is preferably

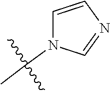 [SMILES: *n1ccnc1]

or hydroxyl.

Other staring compound like

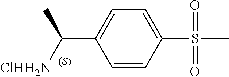

may be used instead of

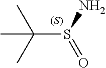

to prepare the corresponding compound, with appropriate modifications of the intermediates.

An exemplary scheme is as follows.

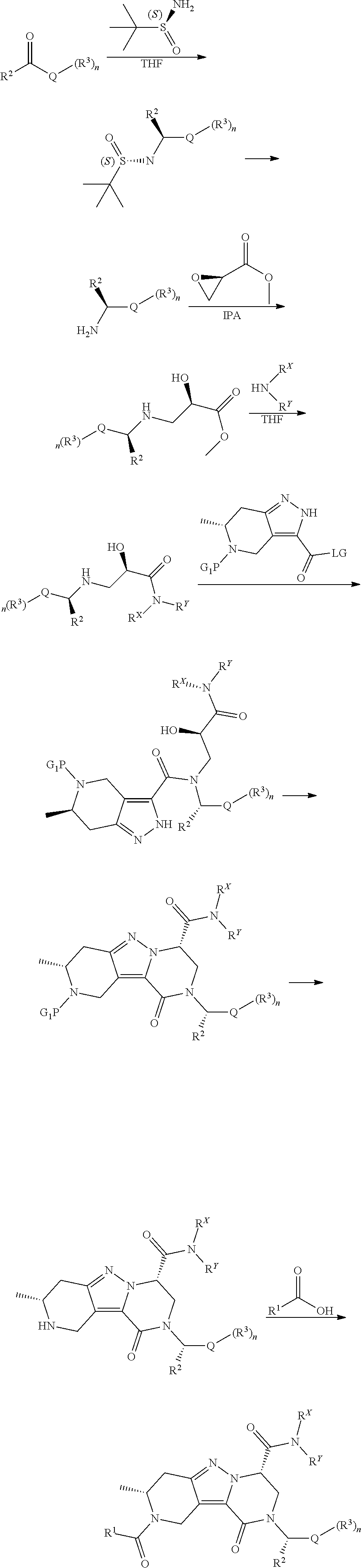

In an exemplary embodiment, the method comprises the step 2′-6′ below instead of steps 2-5:
2′) reacting the compound of Formula (h) with a compound of

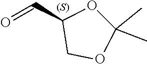

to give a compound of Formula (i′)

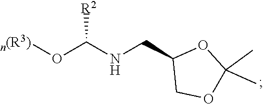

3′) reacting the compound of Formula (i′) with a compound of

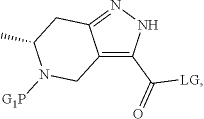

to finally give a compound of Formula (l)

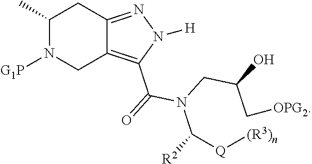

4′) subjecting the compound of Formula (l) to ring closing reaction and deprotection to give a compound of Formula (m)

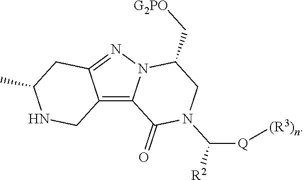

5′) reacting the compound of Formula (m) with a compound of Formula (b)

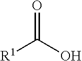

to finally give a compound of Formula (n)

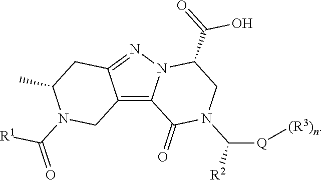

6′) reacting the compound of Formula (n) with a compound of Formula (f)

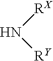

to give the compound of Formula (I′).
wherein R¹, R², R³, Q, n, R^{x} and R^{y} are as defined herein.

In an embodiment, in step 3′), the reaction product of reacting the compound of Formula (i′) with a compound of

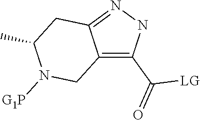

is subjected to deketalization and reacting with a protecting group to give the compound of Formula (l).

In an embodiment, in step 5′), the reaction product of reacting the compound of Formula (m) with a compound of Formula (b)

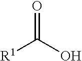

is subjected to deprotection and oxidation to give the compound of Formula (n).

Similarly, PG₁ and PG₂ are each protecting groups, which are conventionally used and may be preferably Boc, TBDPS. LG is a leaving group, which is conventionally used and is preferably

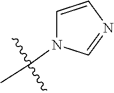 [SMILES: *n1ccnc1]

or hydroxyl.

An exemplary scheme is as follows.

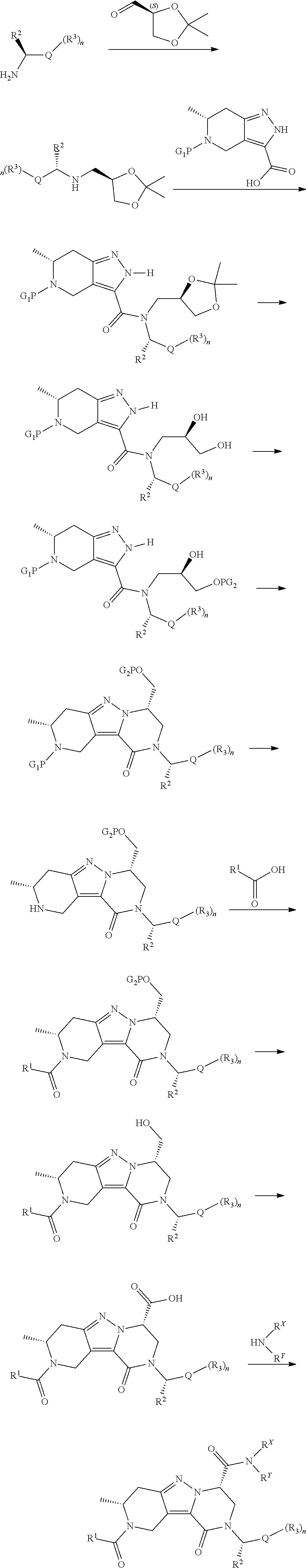

It should be noted that, the chirality of the starting compound may be adjusted so as to obtain the corresponding final product, for example a compound of

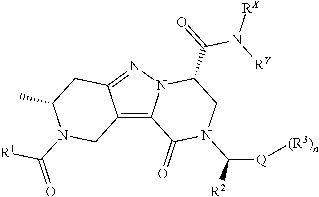

### Formula (I″)

For the preparing methods descried herein, those skilled in the art can modify the starting compounds, reagents, intermediates, sequences and conditions, and/or combine any of the steps or sub-steps according to practical requirements, to give the corresponding final products and such derived methods are also encompassed within the protection scope.

### Definitions

Listed below are definitions of various terms used to describe this present disclosure. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the applicable art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e. to at least one) of the grammatical object of the article. By way of example, “an element” means one element or more than one element. Furthermore, use of the term “including” as well as other forms, such as “include,” “includes,” and “included,” is not limiting.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically acceptable carriers, and excludes other compounds. All ranges disclosed herein are inclusive of the recited endpoint and independently combinable (for example, the range of “from 50 mg to 300 mg” is inclusive of the endpoints, 50 mg and 300 mg, and all the intermediate values). The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “substantially,” cannot be limited to the precise value specified, in some cases. In at least some instances, the approximating language can correspond to the precision of an instrument for measuring the value.

As used herein, the term “at least one” or “one or more” refers to one, two, three, four, five, six, seven, eight, nine or more.

The term “alkyl” as a group or as part of another group, refers to a straight- or branched-chain alkyl group having carbon and hydrogen atoms in the chain. Examples of alkyl groups include methyl (Me, which also may be structurally depicted by the symbol, “/”), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. The term C₁₋₄alkyl as used here refers to a straight- or branched-chain alkyl group having from 1 to 4 carbon atoms in the chain. The term C₁₋₆alkyl as used here refers to a straight- or branched-chain alkyl group having from 1 to 6 carbon atoms in the chain.

The term “alkoxyl” as a group or as part of another group, refers to an alkyl group which is linked to the rest of the molecule via an oxygen, wherein the alkyl is as defined herein. The term C₁₋₄alkoxyl as used here refers to a straight- or branched-chain alkoxyl group having from 1 to 4 carbon atoms in the chain. The term C₁₋₆ alkoxyl as used here refers to a straight- or branched-chain alkoxyl group having from 1 to 6 carbon atoms in the chain. Examples of alkoxyl groups include methoxy, ethoxy, propoxy, butoxy, pentyloxy, hexyloxy, and groups that in light of the ordinary skill in the art and the teachings provided herein would be considered equivalent to any one of the foregoing examples.

The term “C₃₋₆cycloalkyl” refers to a saturated monocyclic carbocycle having from 3 to 6 ring atoms. Illustrative examples of cycloalkyl groups include cyclopropyl, cyclobutyl, cyclopentyl, and cyclohexyl.

The term “phenyl” represents the following moiety.

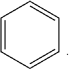

The expression “—NR^{x}R^{y}” herein, alone or in a general formula, refers to

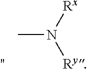

The term “heteroaryl” used herein refers to an aromatic monocyclic or bicyclic aromatic ring system having 5 to 10 ring members and which contains carbon atoms and from 1 to 4 heteroatoms independently selected from the group consisting of N, O, and S. Included within the term heteroaryl are aromatic rings of 5 or 6 members wherein the ring consists of carbon atoms and has at least one (e.g., 1, 2 or 3, preferably 1 or 2) heteroatom member. Suitable heteroatoms include nitrogen (N), oxygen (S), and sulfur (S), preferably nitrogen (N). In the case of 5 membered rings, the heteroaryl ring preferably contains one member of nitrogen, oxygen or sulfur and, in addition, up to 3 additional nitrogens. In the case of 6 membered rings, the heteroaryl ring preferably contains from 1 to 4, e.g. tetrazolyl, more in particular from 1 to 3 nitrogen atoms. For the case wherein the 6 membered ring has 3 nitrogens, at most 2 nitrogen atoms are adjacent. Examples of heteroaryl groups include but not limited to furyl, thienyl, pyrrolyl, oxazolyl, thiazolyl, imidazolyl, pyrazolyl, oxazolyl, thiazolyl, oxadiazolyl, triazolyl, thiadiazolyl, pyridinyl (pyridyl), pyridazinyl, pyrimidinyl, pyrazinyl, indolyl, isoindolyl, benzofuryl, benzothienyl, indazolyl, benzimidazolyl, benzothiazolyl, benzoxazolyl, benzisoxazolyl, benzothiadiazolyl, benzotriazolyl, quinolinyl, isoquinolinyl and quinazolinyl. Unless otherwise noted, the heteroaryl is attached to its pendant group at any heteroatom or carbon atom that results in a stable structure.

Those skilled in the art will recognize that the species of heteroaryl groups listed or illustrated above are not exhaustive, and that additional species within the scope of these defined terms may also be selected.

The term “heterocyclyl” represents a non-aromatic monocyclic or bicyclic system, unless otherwise specified, having for example, 4 to 8 ring members, more usually 5 to 6 ring members. Examples of monocyclic groups are groups containing 4 to 8 ring members, more usually, 5 or 6 ring members. Non-limiting examples of monocyclic heterocyclyl systems containing at least one heteroatom selected from nitrogen, oxygen or sulfur (N, O, S) include, but are not limited to 4- to 8-membered heterocyclyl systems such as oxetanyl, pyrrolidinyl, tetrahydrofuranyl, piperidinyl, piperazinyl, pyranyl, dihydropyranyl, tetrahydropyranyl, morpholinyl, thiomorpholinyl. Unless otherwise specified, each can be bound to the remainder of the molecule through any available ring carbon atom or nitrogen atom, and may optionally be substituted, where possible, on carbon and/or nitrogen atoms according to the embodiments. Optional substituents of 4- to 8-membered monocyclic heterocyclyl, include OH, OC₁₋₄alkyl, halo, COOH, CONHCH₃, NHCOC₁₋₄alkyl, NHCOC₃₋₆cycloalkyl, and C₁₋₄alkyl.

The term “cyano” refers to the group —CN.

The terms “halo” or “halogen” represent chloro (Cl), fluoro (F), bromo (Br) or iodo (I).

The term “oxo” represents ═O.

The term “hydroxyl” represents —OH.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

The terms “para”, “meta”, and “ortho” have the meanings as understood in the art. Thus, for example, a fully substituted phenyl group has substituents at both “ortho” (o) positions adjacent to the point of attachment of the phenyl ring, both “meta” (m) positions, and the one “para” (p) position across from the point of attachment. To further clarify the position of substituents on the phenyl ring, the 2 different ortho positions will be designated as ortho and ortho′ and the 2 different meta positions as meta and meta′ as illustrated below.

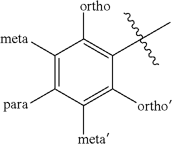

When referring to substituents on a pyridyl group, the terms “para”, “meta”, and “ortho” refer to the placement of a substituent relative to the point of attachment of the pyridyl ring. For example, the structure below is described as 3-pyridyl with the X¹ substituent in the ortho position, the X² substituent in the meta position, and X³ substituent in the para position:

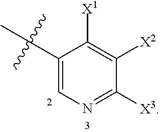

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

The terms “buffered” solution or “buffer” solution are used herein interchangeably according to their standard meaning. Buffered solutions are used to control the pH of a medium, and their choice, use, and function is known to those of ordinary skill in the art. See, for example, G. D. Considine, ed., Van Nostrand's Encyclopedia of Chemistry, p. 261, 5^{th} ed. (2005), describing, inter alia, buffer solutions and how the concentrations of the buffer constituents relate to the pH of the buffer. For example, a buffered solution is obtained by adding MgSO₄ and NaHCO₃ to a solution in a 10:1 w/w ratio to maintain the pH of the solution at about 7.5.

Any formula given herein is intended to represent compounds having structures depicted by the structural formula as well as certain variations or forms. In particular, compounds of any formula given herein may have asymmetric centers and therefore exist in different enantiomeric forms. All optical isomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers.

It is also to be understood that compounds that have the same molecular formula but differ in the nature or sequence of bonding of their atoms or the arrangement of their atoms in space are termed “isomers.”

Stereoisomers that are not mirror images of one another are termed “diastereomers” and those that are non-superimposable mirror images of each other are termed “enantiomers.” When a compound has an asymmetric center, for example, it is bonded to four different groups, and a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either an individual enantiomer or as a mixture thereof. A mixture containing equal proportions of the enantiomers is called a “racemic mixture.”

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenyl nitromethane, that are likewise formed by treatment with acid or base.

Tautomeric forms may be relevant to the attainment of the optimal chemical reactivity and biological activity of a compound of interest.

The compounds of this present disclosure may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

Certain examples contain chemical structures that are depicted as an absolute enantiomer but are intended to indicate enantiopure material that is of unknown configuration. In these cases (R*) or (S*) or (*R) or (*S) is used in the name to indicate that the absolute stereochemistry of the corresponding stereocenter is unknown. Thus, a compound designated as (R*) or (*R) refers to an enantiopure compound with an absolute configuration of either (R) or (S). In cases where the absolute stereochemistry has been confirmed, the structures are named using (R) and (S).

The symbols [symbol] and [symbol] are used as meaning the same spatial arrangement in chemical structures shown herein. Analogously the symbols [symbol] and [symbol] are used as meaning the same spatial arrangement in chemical structures shown herein.

Certain compounds of Formula (I), or pharmaceutically acceptable salts of compounds of Formula (I), may be obtained as solvates. Solvates include those formed from the interaction or complexation of compounds of the present disclosure with one or more solvents, either in solution or as a solid or crystalline form. In some embodiments, the solvent is water and the solvates are hydrates.

Reference to a compound herein stands for a reference to any one of: (a) the actually recited form of such compound, and (b) any of the forms of such compound in the medium in which the compound is being considered when named. For example, reference herein to a compound such as R—COOH, encompasses reference to any one of, for example, R—COOH₍ₛ₎, R—COOH₍ₛₒₗ₎, and R—COO⁻₍ₛₒₗ₎. In this example, R—COOH₍ₛ₎ refers to the solid compound, as it could be for example in a tablet or some other solid pharmaceutical composition or preparation; R—COOH₍ₛₒₗ₎ refers to the undissociated form of the compound in a solvent; and R-COO⁻₍ₛₒₗ₎ refers to the dissociated form of the compound in a solvent, such as the dissociated form of the compound in an aqueous environment, whether such dissociated form derives from R—COOH, from a salt thereof, or from any other entity that yields R—COO⁻ upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form, or forms, of such entity that exists, or exist, in the medium in which such reacting takes place, with (b) the chemically relevant form, or forms, of the compound R—COOH that exists, or exist, in the medium in which such reacting takes place. In this regard, if such entity is for example in an aqueous environment, it is understood that the compound R—COOH is in such same medium, and therefore the entity is being exposed to species such as R—COOH_{(aq)} and/or R-COO⁻_{(aq)}, where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

In another example, a zwitterionic compound is encompassed herein by referring to a compound that is known to form a zwitterion, even if it is not explicitly named in its zwitterionic form. Terms such as zwitterion, zwitterions, and their synonyms zwitterionic compound(s) are standard IUPAC-endorsed names that are well known and part of standard sets of defined scientific names. In this regard, the name zwitterion is assigned the name identification CHEBI:27369 by the Chemical Entities of Biological Interest (ChEBI) dictionary of molecular entities. As generally well known, a zwitterion or zwitterionic compound is a neutral compound that has formal unit charges of opposite sign. Sometimes these compounds are referred to by the term “inner salts”. Other sources refer to these compounds as “dipolar ions”, although the latter term is regarded by still other sources as a misnomer. As a specific example, aminoethanoic acid (the amino acid glycine) has the formula H₂NCH₂COOH, and it exists in some media (in this case in neutral media) in the form of the zwitterion ⁺H₃NCH₂COO⁻. Zwitterions, zwitterionic compounds, inner salts and dipolar ions in the known and well established meanings of these terms are within the scope of this present disclosure, as would in any case be so appreciated by those of ordinary skill in the art. Because there is no need to name each and every embodiment that would be recognized by those of ordinary skill in the art, no structures of the zwitterionic compounds that are associated with the compounds of this present disclosure are given explicitly herein. They are, however, part of the embodiments of this present disclosure. No further examples in this regard are provided herein because the interactions and transformations in a given medium that lead to the various forms of a given compound are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorus, sulfur, fluorine, chlorine, and iodine such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, ¹²⁵I, respectively. Such isotopically labeled compounds are useful in metabolic studies (preferably with ¹⁴C), reaction kinetic studies (with, for example deuterium (i.e., D or ²H); or tritium (i.e., T or ³H)), detection or imaging techniques such as positron emission tomography (PET) or single-photon emission computed tomography (SPECT) including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this present disclosure can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this present disclosure given according to the choices S¹ₑₓₐₘₚₗₑ is S₁ and S²ₑₓₐₘₚₗₑ is S₃; S¹ₑₓₐₘₚₗₑ is S₁ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent assignments described herein. The foregoing convention given herein for substituents extends, when applicable, to members such as R¹, R², R³, R⁴, R⁵, G¹, G², G³, G⁴, G⁵, G⁶, G⁷, G⁸, G⁹, G¹⁰, G¹¹, n, L, R, T, Q, W, X, Y, and Z and any other generic substituent symbol used herein.

Furthermore, when more than one assignment is given for any member or substituent, embodiments of this present disclosure comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this present disclosure for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent assignments described herein. The foregoing convention given herein for substituents extends, when applicable, to members such as R¹, R², R³, R⁴, R⁵, G¹, G², G³, G⁴, G⁵, G⁶, G⁷, G⁸, G⁹, G¹⁰, G¹¹, n, L, R, T, Q, W, X, Y, and Z and any other generic substituent symbol used herein.

The nomenclature “Cᵢ₋ⱼ” with j>i, when applied herein to a class of substituents, is meant to refer to embodiments of this present disclosure for which each and every one of the number of carbon members, from i to j including i and j, is independently realized. By way of example, the term C₁₋₆ refers independently to embodiments that have one carbon member (C₁), embodiments that have two carbon members (C₂), embodiments that have three carbon members (C₃), and embodiments that have four carbon members (C₄), embodiments that have five carbon members (C₅), and embodiments that have six carbon members (C₆).

The term Cₙ₋ₘalkyl refers to an aliphatic chain, whether straight or branched, with a total number N of carbon members in the chain that satisfies n N<m, with m>n. Any disubstituent referred to herein is meant to encompass the various attachment possibilities when more than one of such possibilities are allowed. For example, reference to disubstituent -A-B—, where A≠B, refers herein to such disubstituent with A attached to a first substituted member and B attached to a second substituted member, and it also refers to such disubstituent with A attached to the second substituted member and B attached to the first substituted member.

The present disclosure includes also pharmaceutically acceptable salts of the compounds of Formula (I), preferably of those described above and of the specific compounds exemplified herein, and methods of treatment using such salts.

The term “pharmaceutically acceptable” means approved or approvable by a regulatory agency of Federal or a state government or the corresponding agency in countries other than the United States, or that is listed in the U. S. Pharmacopoeia or other generally recognized pharmacopoeia for use in animals, and more particularly, in humans.

A “pharmaceutically acceptable salt” is intended to mean a salt of a free acid or base of compounds represented by Formula (I) that are non-toxic, biologically tolerable, or otherwise biologically suitable for administration to the subject. It should possess the desired pharmacological activity of the parent compound. See, generally, G. S. Paulekuhn, et al., “Trends in Active Pharmaceutical Ingredient Salt Selection based on Analysis of the Orange Book Database”, J Med. Chem., 2007, 50:6665-72, S. M. Berge, et al., “Pharmaceutical Salts”, J Pharm Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt.

As used herein, the term “composition” or “pharmaceutical composition” refers to a mixture of at least one compound provided herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound provided herein within or to the patient such that it can perform its intended function. Typically, such constructs are carried or transported from one organ, or portion of the body, to another organ, or portion of the body. Each carrier must be “acceptable” in the sense of being compatible with the other ingredients of the formulation, including the compound provided herein, and not injurious to the patient. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound provided herein, and are physiologically acceptable to the patient. Supplementary active compounds can also be incorporated into the compositions. The “pharmaceutically acceptable carrier” can further include a pharmaceutically acceptable salt of the compound provided herein. Other additional ingredients that can be included in the pharmaceutical compositions provided herein are known in the art and described, for example in Remington's Pharmaceutical Sciences (Genaro, Ed., Mack Publishing Co., 1985, Easton, PA), which is incorporated herein by reference.

The term “stabilizer,” as used herein, refers to polymers capable of chemically inhibiting or preventing degradation of a compound disclosed herein. Stabilizers are added to formulations of compounds to improve chemical and physical stability of the compound.

The term “tablet,” as used herein, denotes an orally administrable, single-dose, solid dosage form that can be produced by compressing a drug substance or a pharmaceutically acceptable salt thereof, with suitable excipients (e.g., fillers, disintegrants, lubricants, glidants, and/or surfactants) by conventional tableting processes.

As used herein, the term “capsule” refers to a solid dosage form in which the drug is enclosed within either a hard or soft soluble container or “shell.” The container or shell can be formed from gelatin, starch and/or other suitable substances.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

The term “combination,” “therapeutic combination,” “pharmaceutical combination,” or “combination product” as used herein refer to a non-fixed combination or a kit of parts for the combined administration where two or more therapeutic agents can be administered independently, at the same time or separately within time intervals, especially where these time intervals allow that the combination partners show a cooperative, e.g., synergistic, effect.

The term “modulators” include both inhibitors and activators, where “inhibitors” refer to compounds that decrease, prevent, inactivate, desensitize, or down-regulate HBV assembly and other HBV core protein functions necessary for HBV replication or the generation of infectious particles.

As used herein, the term “capsid assembly modulator” refers to a compound that disrupts or accelerates or inhibits or hinders or delays or reduces or modifies normal capsid assembly (e.g., during maturation) or normal capsid disassembly (e.g., during infectivity) or perturbs capsid stability, thereby inducing aberrant capsid morphology and function. In one embodiment, a capsid assembly modulator accelerates capsid assembly or disassembly, thereby inducing aberrant capsid morphology. In another embodiment, a capsid assembly modulator interacts (e.g. binds at an active site, binds at an allosteric site, modifies and/or hinders folding and the like) with the major capsid assembly protein (CA), thereby disrupting capsid assembly or disassembly. In yet another embodiment, a capsid assembly modulator causes a perturbation in structure or function of CA (e.g., ability of CA to assemble, disassemble, bind to a substrate, fold into a suitable conformation, or the like), which attenuates viral infectivity and/or is lethal to the virus.

As used herein, the term “treatment” or “treating,” is defined as the application or administration of a therapeutic agent, i.e., a compound of the present disclosure (alone or in combination with another pharmaceutical agent), to a patient, or application or administration of a therapeutic agent to an isolated tissue or cell line from a patient (e.g., for diagnosis or ex vivo applications), who has an HBV infection, a symptom of HBV infection or the potential to develop an HBV infection, with the purpose to cure, heal, alleviate, relieve, alter, remedy, ameliorate, improve or affect the HBV infection, the symptoms of HBV infection or the potential to develop an HBV infection. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human or a non-human mammal. Non-human mammals include, for example, livestock and pets, such as ovine, bovine, porcine, canine, feline and murine mammals. Preferably, the patient, subject or individual is human.

In treatment methods according to the present disclosure, an effective amount of a pharmaceutical agent according to the present disclosure is administered to a subject suffering from or diagnosed as having such a disease, disorder, or condition. An “effective amount” means an amount or dose sufficient to generally bring about the desired therapeutic or prophylactic benefit in patients in need of such treatment for the designated disease, disorder, or condition. Effective amounts or doses of the compounds of the present disclosure may be ascertained by routine methods such as modeling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.001 to about 200 mg of compound per kg of subject's body weight per day. An example of a dose of a compound is from about 1 mg to about 2,500 mg.

Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

HBV infections that may be treated according to the disclosed methods include HBV genotype A, B, C, and/or D infections. However, in an embodiment, the methods disclosed may treat any HBV genotype (“pan-genotypic treatment”). HBV genotyping may be performed using methods known in the art, for example, INNO-LIPA® HBV Genotyping, Innogenetics N.V., Ghent, Belgium).

Some exemplary embodiments are provided as follows.

Embodiment 1. A compound of Formula (I),

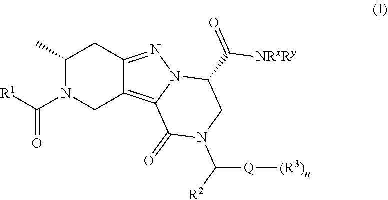

or a stereoisomeric or a tautomeric form thereof, wherein
R¹ is selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₃₋₆cycloalkyl, CN, CF₃, CHF₂, OCHF₂ and OCF₃;
R² is selected from the group consisting of H, CHF₂, CF₃, C₁₋₆alkyl, C₁₋₆alkylOC₁₋₆alkyl and C₃₋₆cycloalkyl;
Q represents a ring selected from the group consisting of phenyl, 5-membered heteroaryl and 6-membered heteroaryl;
n represents 1, 2 or 3;
each R³ independently represents a substituent selected from the group consisting of CN, C₁₋₆alkyl, C₁₋₆alkoxyl, C₃₋₆cycloalkyl, 5-membered heteroaryl, 6-membered heteroaryl, 4-8 membered heterocyclyl and halo, each of C₁₋₆alkyl, C₁₋₆alkoxyl, C₃₋₆cycloalkyl, 5-membered heteroaryl, 6-membered heteroaryl and 4-8 membered heterocyclyl is optionally substituted with 1, 2, 3, 4 or 5 substituents, each of said substituents independently selected from the group consisting of halo, hydroxyl, C₁₋₆alkyl and oxo;
Rₓ and R_{y} are each independently selected from the group consisting of hydrogen and C₁₋₆alkyl;
wherein the following compounds are excluded:

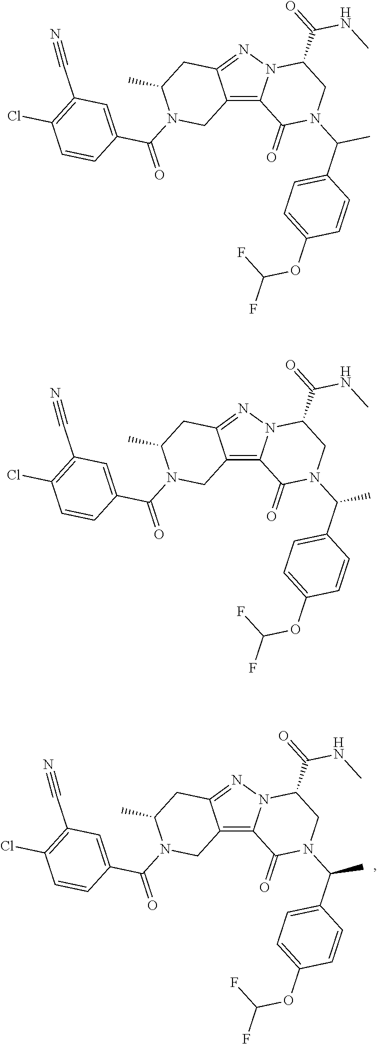 [SMILES: CNC(=O)[C@@H]1CN(C(C)c2ccc(OC(F)F)cc2)C(=O)c2c3c(nn21)C[C@@H](C)N(C(=O)c1ccc(Cl)c(C#N)c1)C3 ; CNC(=O)[C@@H]1CN([C@H](C)c2ccc(OC(F)F)cc2)C(=O)c2c3c(nn21)C[C@@H](C)N(C(=O)c1ccc(Cl)c(C#N)c1)C3 ; CNC(=O)[C@@H]1CN([C@@H](C)c2ccc(OC(F)F)cc2)C(=O)c2c3c(nn21)C[C@@H](C)N(C(=O)c1ccc(Cl)c(C#N)c1)C3]

or a pharmaceutically acceptable salt or a solvate thereof.

Embodiment 2. The compound of Embodiment 1, wherein R¹ is a ring selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₃₋₆cycloalkyl, CF₃, CHF₂, OCHF₂ and OCF₃;
preferably, R¹ is a ring selected from the group consisting of phenyl, pyridyl, pyrimidinyl, pyridazinyl and pyrazinyl, each of which is substituted with 1, 2 or 3 substituents, each of said substituents independently selected from the group consisting of halo, C₁₋₆alkyl, C₃₋₆cycloalkyl, CF₃, CHF₂, OCHF₂ and OCF₃.

Embodiment 3. The compound of any one of the preceding Embodiments, wherein R¹ is phenyl, which is substituted with 1, 2 or 3 substituents, each of said substituents independently selected from the group consisting of halo, C₁₋₆alkyl, CF₃, and CHF₂.

Embodiment 4. The compound of Embodiment 1, wherein the structural unit

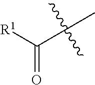

in Formula (I) satisfies Formula (Ia)

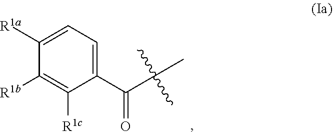

wherein R^{1a}, R^{1b}, and R^{1c}, each independently are selected from the group consisting of hydrogen, halo, C₁₋₆alkyl, C₃₋₆cycloalkyl, CN, CF₃, CHF₂, OCHF₂ and OCF₃, with at least one of R^{1a}, R^{1l}, and R^{1c} not being hydrogen;
preferably wherein R^{1a}, R^{1b}, and R^{1c}, each independently are selected from the group consisting of hydrogen, halo, C₁₋₆alkyl, C₃₋₆cycloalkyl, CF₃, CHF₂, OCHF₂ and OCF₃, with at least one of R^{1a}, R^{1b}, and R^{1c} not being hydrogen.

Embodiment 5. The compound of Embodiment 4, wherein R^{1a} and R^{1b} are independently selected from the group consisting of halo and CF₃, and wherein R^{1c} is hydrogen.

Embodiment 6. The compound of any one of the preceding Embodiments, wherein R² is selected from the group consisting of CHF₂, CF₃, C₁₋₆alkyl, C₁₋₆alkylOC₁₋₆alkyl and C₃₋₆cycloalkyl;
the structure of Formula (I) has Formula (I-1) or Formula (I-2)

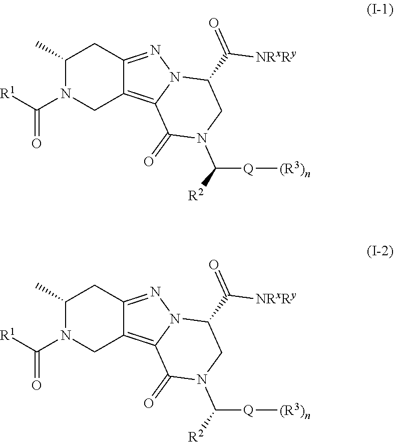

Embodiment 7. The compound of any one of the preceding Embodiments, wherein R² is methyl.

Embodiment 8. The compound of any one of the preceding Embodiments, wherein Q is a ring selected from the group consisting of phenyl, pyridyl, pyrimidinyl, pyridazinyl, pyrazinyl, pyrazolyl, and imidazolyl.

Embodiment 9. The compound of any one of the preceding Embodiments, wherein the structural unit

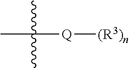

in Formula (I) satisfies Formula (Ib)

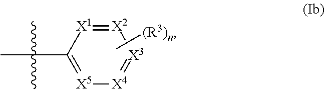

wherein
all of X¹, X², X³, X⁴ and X⁵ are CH; or
one or two of X¹, X², X³, X⁴ and X⁵ are N, and rest of them are CH.

Embodiment 10. The compound of Embodiment 9, wherein the structural unit

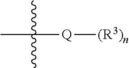

of Formula (I) satisfies Formula (Ic)

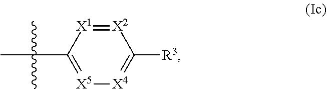

wherein
all of X¹, X², X⁴ and X⁵ are CH;
X² is N, and X¹, X⁴ and X⁵ are CH; or
X¹ is N, and X², X⁴ and X⁵ are CH.

Embodiment 11. The compound of Embodiment 9, wherein
both of X¹ and X² are N, and X⁴ and X⁵ are CH;
both of X² and X⁴ are N, and X¹ and X⁵ are CH;
both of X¹ and X⁴ are N, and X² and X⁵ are CH; or
both of X¹ and X⁵ are N, and X² and X⁴ are CH.

Embodiment 12. The compound of any one of the preceding Embodiments, wherein the structural unit

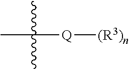

in Formula (I) of satisfies Formula (Ib′)

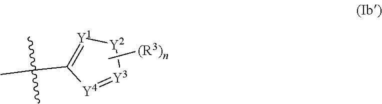

wherein one or two of Y¹, Y², Y³ and Y⁴ are N or NH, and rest of them are C.

Embodiment 13. The compound of any one of the preceding Embodiments, wherein halo is F, Cl or Br.

Embodiment 14. The compound of any one of the preceding Embodiments, wherein n is 1 or 2.

Embodiment 15. The compound of any one of the preceding Embodiments, wherein one of R^{x} and R^{y} is hydrogen, and the other is C₁₋₆alkyl.

Embodiment 16. The compound of any one of the preceding Embodiments, wherein one R³ is independently selected from the group consisting of halo, CH₃,

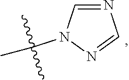 [SMILES: *n1cncn1]

C(CH₃)₂OH, CH₂CF₃, CF₃ and OCHF₂.

Embodiment 17. A compound selected from the group consisting of the compounds in Table 1 and Table 2 and Examples, or a stereoisomeric or a tautomeric form thereof:
or a pharmaceutically acceptable salt, N-oxide, or a solvate thereof.

Embodiment 18. A pharmaceutical composition, which comprises the compound of any one of Embodiments 1 to 17, and which further comprises at least one pharmaceutically acceptable excipient.

Embodiment 19. The compound of any one of Embodiments 1 to 17, or the pharmaceutical composition of Embodiment 18, for use as a medicament.

Embodiment 20. The compound of any one of Embodiments 1 to 17, or the pharmaceutical composition of Embodiment 18, for use in the prevention or treatment of an HBV infection or of an HBV-induced disease in a subject in need thereof.

Embodiment 21. The compound of any one of Embodiments 1 to 17, or the pharmaceutical composition of Embodiment 18, for use in the prevention or treatment of chronic hepatitis B.

Embodiment 22. A method of treating an HBV infection or an HBV-induced disease in an individual in need thereof, comprising administering to the individual a therapeutically effective amount of the compound of any one of Embodiments 1 to 17 or the pharmaceutical composition of Embodiment 18.

Embodiment 23. A product comprising a first compound and a second compound as a combined preparation for simultaneous, separate or sequential use in the prevention or treatment of an HBV infection or of an HBV-induced disease in a subject in need thereof, wherein said first compound is different from said second compound, wherein said first compound is the compound of any one of Embodiments 1 to 17 or the pharmaceutical composition of Embodiment 18, and wherein said second compound is another HBV inhibitor.

Embodiment 24. The product of Embodiment 23, wherein said second compound is another HBV inhibitor which is selected from the group consisting of: therapeutic agents selected from HBV combination drugs, HBV vaccines, HBV DNA polymerase inhibitors, immunomodulatory agents, toll-like receptor (TLR) modulators, interferon alpha receptor ligands, hyaluronidase inhibitors, hepatitis b surface antigen (HBsAg) inhibitors, cytotoxic T-lymphocyte-associated protein 4 (CTLA-4) inhibitors, cyclophilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, famesoid X receptor agonists, HBV antibodies, CCR2 chemokine antagonists, thymosin agonists, cytokines, nucleoprotein modulators, retinoic acid-inducible gene 1 simulators, NOD2 stimulators, phosphatidylinositol 3-kinase (PI3K) inhibitors, indoleamine-2, 3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and other HBV drugs.

Embodiment 25. A compound as defined in any one of Embodiments 1 to 17 or the pharmaceutical composition of claim 18 for use in the prevention or treatment of an HBV infection or an HBV-induced disease in a subject, wherein the compound or pharmaceutical composition is administered to the subject in combination with another HBV inhibitor.

In an attempt to help the reader of the present application, the description has been separated in various paragraphs or sections. These separations should not be considered as disconnecting the substance of a paragraph or section from the substance of another paragraph or section. To the contrary, the present description encompasses all the combinations of the various sections, paragraphs and sentences that can be contemplated.

Each of the relevant disclosures of all references cited herein is specifically incorporated by reference. The following examples are offered by way of illustration, and not by way of limitation.

### Examples

Exemplary compounds useful in methods of the present disclosure will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

Compounds of any one of Formula (I), may be converted to their corresponding salts using methods known to one of ordinary skill in the art. For example, an amine of Formula (I) is treated with trifluoroacetic acid, HCl, or citric acid in a solvent such as Et₂O, CH₂Cl₂, THF, MeOH, chloroform, or isopropanol to provide the corresponding salt form. Alternately, trifluoroacetic acid or formic acid salts are obtained as a result of reverse phase HPLC purification conditions. Crystalline forms of pharmaceutically acceptable salts of compounds of Formula (I), may be obtained in crystalline form by recrystallization from polar solvents (including mixtures of polar solvents and aqueous mixtures of polar solvents) or from non-polar solvents (including mixtures of non-polar solvents).

Where the compounds according to this present disclosure have at least one chiral center, they may accordingly exist as enantiomers. Where the compounds possess two or more chiral centers, they may additionally exist as diastereomers. It is to be understood that all such isomers and mixtures thereof are encompassed within the scope of the present disclosure.

Compounds represented as “stereomeric mixture” (means a mixture of two or more stereoisomers and includes enantiomers, diastereomers and combinations thereof) are separated by SFC resolution.

Compounds may be obtained as single forms, such as single enantiomers, by form-specific synthesis, or by resolution. Compounds may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

### 1. General Information

### Chemical Names

Chemical names were generated using the chemistry software: ACD/ChemSketch, and may follow preferably the IUPAC rules.

### General Procedure for LCMS Methods

The High Performance Liquid Chromatography (HPLC) measurement was performed using a LC pump, a diode-array (DAD) or a UV detector and a column as specified in the respective methods.

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW).

Data acquisition was performed with appropriate software.

Compounds are described by their experimental retention times (Rt) and ions. If not specified differently, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc.). All results were obtained with experimental uncertainties that are commonly associated with the method used. Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica., “Q-Tof” Quadrupole Time-of-flight mass spectrometers, “CLND”, ChemiLuminescent Nitrogen Detector, “ELSD” Evaporative Light Scanning Detector.

### NMR Analysis

¹H NMR spectra were recorded on 1) a Bruker DPX 400 MHz spectrometer or 2) a Bruker Avance 400 MHz spectrometer or c) Bruker Avance III 400 MHz spectrometer or d) Bruker Avance 600 MHz spectrometer or e) a Bruker Avance NEO 400 MHz spectrometer or f) Bruker model AVIII 400 spectrometer g) ZKNJ BIXI-1 300 MHz, Bruker Avance III 400 MHz or h) Bruker AVANCE Neo 400 MHz.

NMR spectra were recorded at ambient temperature unless otherwise stated. Data are reported as follow: chemical shift in parts per million (ppm) relative to TMS (6=0 ppm) on the scale, integration, multiplicity (s=singlet, d=doublet, t=triplet, q=quartet, quin=quintet, sext=sextet, sept=septet, m=multiplet, b=broad, or a combination of these), coupling constant(s) Jin Hertz (Hz).

### MS Analysis

Mass spectra were obtained on a Shimadzu LCMS-2020 MSD or Agilent 1200/G6110A MSD using electrospray ionization (ESI) in positive mode unless otherwise indicated.

### 2. Abbreviations

TABLE 3

| | |
|---|---|
| μw | Microwave |
| ADDP | 1,1′-(Azodicarbonyl)dipiperidine |
| AIBN | 2,2′-azobis(2-methylpropionitrile) |
| AlMe₃ | Trimethylaluminium |
| aq. | Aqueous |
| atm | Atmosphere |
| Boc₂O | Di-tert-butyl dicarbonate |
| BOC | tert-Butyloxycarbonyl |
| BODIPY | Boron-dipyrromethene |
| BPO | Dibenzoyl peroxide |
| br | broad |
| CA | Capsid assembly |
| CDI | Di(1H-imidazol-1-yl)methanone |
| DAST | (Diethylamino)sulfur trifluoride |
| DBAD | Di-tert-butyl azodicarboxylate |
| DBU | 1,8-Diazabicyclo[5.4.0]undec-7-ene |
| DCE | 1,2-Dichloroethane |
| DCM | Dichloromethane |
| dd | Doublet of doublets |
| DEA | Diethylamine |
| DIAD | (E)-diisopropyl diazene-1,2-dicarboxylate |
| DIPE | Diisopropyl ether |
| DIPEA/DIEA | N,N-diisopropylethylamine |
| DMAP | N,N-dimethylpyridin-4-amine |
| DMF | Dimethylformamide |
| DMF-DMA | Dimethylformamide-dimethylacetal |
| DMSO | Dimethylsulfoxide |
| DNA | Deoxyribonucleic Acid |
| EDCI | N-(3-Dimethylaminopropyl)-N′-ethylcarbodiimide |
| | hydrochloride |
| ESI | Electrospray ionization |
| Et₃N | Triethylamine |
| Et₂O | Ether, diethyl ether |
| EtOAc/EA | Ethyl acetate |
| EtOH | Ethanol |
| FCC | Normal-phase silica gel chromatography |
| g | grams |
| h/hr | hour |
| HATU | 2-(7-Aza-1H-benzotriazole-1-yl)-1,1,3,3- |
| | tetramethyluronium hexafluorophosphate |
| HBV | Hepatitis B virus |
| HOAc | Acetic acid |
| HOBT | Benzotriazol-1-ol |
| HPLC | High Performance Liquid Chromatography |
| Hz | Hertz |
| i-PrNH₂ | Isopropylamine |
| i-PrOH/IPA | Isopropyl alcohol |
| KOtBu | Potassium tert-butoxide |
| LAH | Lithium aluminum hydride |
| LCMS | Liquid Chromatography Mass Spectrometry |
| LHMDS, | Lithium bis(trimethylsilyl)amide |
| LiHMDS | |
| M | molar |
| m | multiplet |
| MeCN/ACN | Acetonitrile |
| MeOH | Methanol |
| mg | milligrams |
| MHz | megahertz |
| min | Minutes |
| mL | Milliliters |
| μL | microliters |
| mmol | Millimole |
| μmol | micromole |
| MS | Mass spectra |
| MsCl | Mesityl chloride |
| m/z | Mass to charge ratio |
| N | normal |
| NaOAc/AcONa | Sodium acetate |
| NBS | N-Bromosuccinimide |
| NMR | Nuclear Magnetic Resonance |
| NMP | 1-methylpyrrolidin-2-one |
| o/n | Overnight |
| PCR | Polymerase chain reaction |
| PE | Petroleum ether |
| PMPA | 9-(2-Phosphonyl-methoxypropyl)adenine |
| ppm | Parts per million |
| ppt | precipitate |
| Py | pyridine |
| RNA | Ribonucleic Acid |
| Rₜ | Retention time |
| rt | Room temperature |
| s | singlet |
| sat. | Saturated |
| SFC | Supercritical Fluid Chromatography |
| t | triplet |
| T₃P | Propanephosphonic acid anhydride |
| TBAF | Tetrabutylammonium fluoride |
| TBAI | Tetrabutylammonium iodide |
| TBD | 1,5,7-Triazabicyclo[4.4.0]dec-5-ene |
| TBDPSCl | tert-butylchlorodiphenylsilane |
| TBDPS | tert-butyldiphenylsilyl |
| TEA | triethylamine |
| TEBAC | N-benzyl-N,N-diethylethanaminium chloride |
| TEMPO | 2,2,6,6-Tetramethylpiperidinooxy |
| TFA | Trifluoroacetic acid |
| THF | Tetrahydrofuran |
| TLC | Thin Layer Chromatography |
| TLR | Toll-like receptor |
| TNF | Tumor necrosis factor |
| V or volumes | Volume in milliliters of solvent per gram of substrate |
| Δ | Heating under reflux |
| Cbz | benzyloxycarbonyl |

### Experimental Procedures

Intermediate Int A

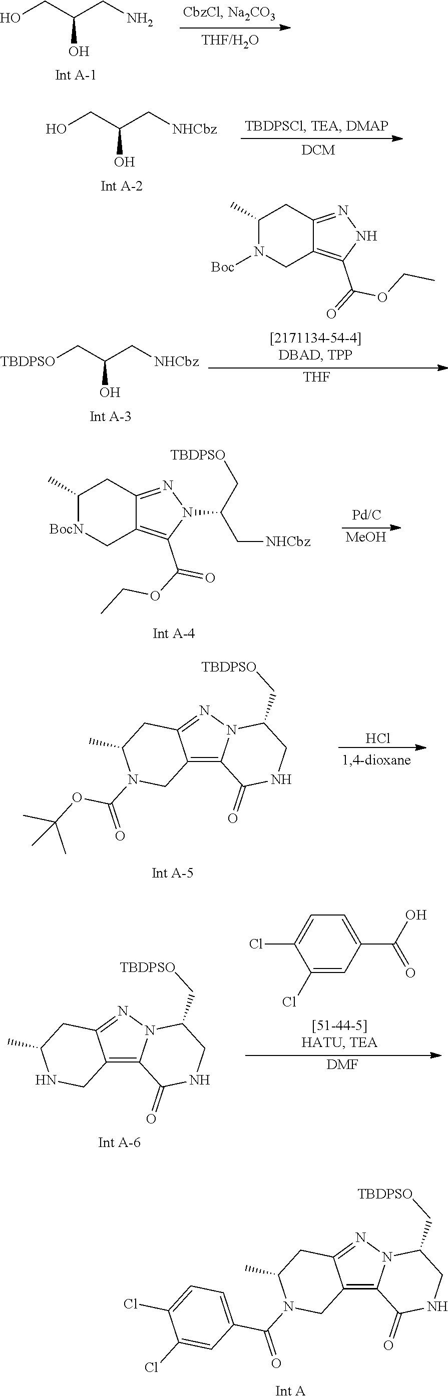

Intermediate Int A-2

(R)-Benzyl (2,3-dihydroxypropyl)carbamate

To a solution of (R)-3-aminopropane-1,2-diol Int A-1 (5 g, 54.9 mmol) in tetrahydrofuran (30 mL) and water (30 mL) was added sodium carbonate (8 g, 75.5 mmol), and dropwise benzyl chloroformate (8.3 mL, 58.4 mmol) at 0° C. under nitrogen atmosphere. After being stirred at room temperature overnight, the mixture was filtered, removed tetrahydrofuran and extracted with ethyl acetate (30 mL) three times. The combined organic layers were washed with brine (30 mL), dried over Na₂SO₄₍ₛ₎, filtered and concentrated to give the title compound (11 g, 90% purity from ¹H NMR, 80% yield) as white solids. ¹H NMR (300 MHz, CDCl₃) 7.48-7.35 (m, 5H), 5.27 (br s, 1H), 5.16 (br s, 2H), 3.89-3.78 (m, 1H), 3.73-3.60 (m, 2H), 3.47-3.31 (m, 2H), 2.47-2.28 (m, 2H).

Intermediate Int A-3

(R)-Benzyl (3-((tert-butyldiphenylsilyl)oxy)-2-hydroxypropyl)carbamate

To a solution of (R)-benzyl (2,3-dihydroxypropyl)carbamate Int A-2 (11 g, 90% purity, 44.0 mmol) in dichloromethane (110 mL) was added triethylamine (6.6 mL, 78.3 mmol), N,N-dimethylpyridin-4-amine (247 mg, 2.02 mmol) and tert-butylchlorodiphenylsilane (12 mL, 46.1 mmol) at 0° C. After being stirred at room temperature overnight, the mixture was concentrated, filtered, and washed with dichloromethane. The filtrate was concentrated and purified by silica gel column chromatography (petroleum ether:ethyl acetate=5:1 to 2:1) to give the title compound (17 g, 90% purity from ¹H NMR, 75% yield) as a colorless oil. ¹H NMR (400 MHz, CDCl₃) 7.64-7.62 (m, 4H), 7.45-7.30 (m, 11H), 5.08 (s, 3H), 3.84-3.79 (m, 1H), 3.70-3.58 (m, 2H), 3.45-3.39 (m, 1H), 3.23-3.16 (m, 1H), 1.06 (s, 9H).

Intermediate Int A-4

(R)-5-tert-Butyl 3-ethyl 2-((R)-10,10-dimethyl-3-oxo-1,9,9-triphenyl-2,8-dioxa-4-aza-9-silaundecan-6-yl)-6-methyl-6,7-dihydro-2H-pyrazolo[4,3-c]pyridine-3,5(4H)-dicarboxylate

To a solution of (R)-benzyl (3-((tert-butyldiphenylsilyl)oxy)-2-hydroxypropyl)carbamate Int A-3 (17 g, 90% purity, 33.0 mmol), (R)-5-tert-butyl 3-ethyl 6-methyl-6,7-dihydro-2H-pyrazolo[4,3-c]pyridine-3,5(4H)-dicarboxylate (15 g, 48.5 mmol) and triphenylphosphine (17 g, 64.8 mmol) in tetrahydrofuran (300 mL) was added di-tert-butyl azodicarboxylate (15 g, 65.1 mmol) at 0° C. After being stirred at 50° C. overnight, the mixture was concentrated and purified by silica gel column chromatography (petroleum ether:ethyl acetate=5:1 to 2:1) to give the crude product, which was further purified by C18 column (acetonitrile:water=95% to 100%) to give the title compound (21 g, 82% purity from LCMS, 69% yield) as a pale yellow oil. LC-MS (ESI): R_{T}=1.40 min, mass calcd. for C₄₂H₅₄N₄O₇Si 754.4, m/z found 755.1 [M+H]⁺.

Intermediate Int A-5

(3R,7S)-tert-Butyl 7-(((tert-butyldiphenylsilyl)oxy)methyl)-3-methyl-10-oxo-3,4,7,8,9,10-hexahydropyrido[4′,3′:3,4]pyrazolo[1,5-a]pyrazine-2(1H)-carboxylate

To a solution of (R)-5-tert-butyl 3-ethyl 2-((R)-10,10-dimethyl-3-oxo-1,9,9-triphenyl-2,8-dioxa-4-aza-9-silaundecan-6-yl)-6-methyl-6,7-dihydro-2H-pyrazolo[4,3-c]pyridine-3,5(4H)-dicarboxylate Int A-4 (13 g, 82% purity, 14.1 mmol) in methanol (150 mL) was added 10% palladium on activated carbon catalyst (1.5 g, 1.41 mmol). After being stirred at room temperature under hydrogen atmosphere overnight, the reaction mixture was filtrated. The filtrate was concentrated and purified by silica gel column chromatography (petroleum ether: ethyl acetate=5:1 to 2:1) to give the title compound (6.7 g, 100% purity from ¹H NMR, 83% yield) as pale yellow solids. LC-MS (ESI): R_{T}=2.03 min, mass calcd. for C₃₂H₄₂N₄O₄Si 574.3, m/z found 575.1 [M+H]⁺. ¹H NMR (400 MHz, DMSO-d₆) 8.13 (br s, 1H), 7.56-7.51 (m, 4H), 7.48-7.40 (m, 6H), 4.90 (d, J=17.2 Hz, 1H), 4.77-4.65 (m, 1H), 4.58-4.54 (m, 1H), 4.12-3.83 (m, 4H), 3.71-3.65 (m, 1H), 2.83 (dd, J=15.6, 6.0 Hz, 1H), 2.56 (d, J=15.6 Hz, 1H), 1.43 (s, 9H), 1.00 (d, J=6.8 Hz, 3H), 0.92 (s, 9H).

Intermediate Int A-6

(3R,7S)-7-(((tert-Butyldiphenylsilyl)oxy)methyl)-3-methyl-1,2,3,4,8,9-hexahydropyrido[4′,3′:3,4]pyrazolo[1,5-a]pyrazin-10(7H)-one hydrochloride

A solution of (3R,7S)-tert-butyl 7-(((tert-butyldiphenylsilyl)oxy)methyl)-3-methyl-10-oxo-3,4,7,8,9,10-hexahydropyrido[4′,3′:3,4]pyrazolo[1,5-a]pyrazine-2(1H)-carboxylate Int A-5 (1.36 g, 2.37 mmol) in 4 M hydrochloride in 1,4-dioxane (20 mL) was stirred at room temperature for 2 hours. The mixture was concentrated under reduced pressure to give the title compound (1.2 g, 93% purity, 92.3% yield) as yellow solids. LC-MS (ESI): R_{T}=1.34 min, mass calcd. for C₂₇H₃₄N₄O₂Si 474.3, m/z found 474.7 [M+H]⁺.

Intermediate Int A

(3R,7S)-7-(((tert-Butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one

To the solution of (3R,7S)-7-(((tert-butyldiphenylsilyl)oxy)methyl)-3-methyl-1,2,3,4,8,9-hexahydropyrido[4′,3′:3,4]pyrazolo[1,5-a]pyrazin-10(7H)-one Int A-6 (1.5 g, 97% purity, 3.07 mmol) in N,N-dimethylformamide (15 mL) was added 3,4-dichlorobenzoic acid (800 mg, 4.19 mmol), 2-(7-Aza-1H-benzotriazole-1-yl)-1,1,3,3-tetramethyluronium hexafluorophosphate (1.8 g, 4.73 mmol) and triethylamine (1.5 mL, 10.8 mmol) at 0° C. The mixture was stirred at room temperature for 2 hours. The mixture was diluted with water (50 mL), acidized with 0.5 M hydrochloride aqueous solution to pH˜6 and extracted with ethyl acetate (80 mL) twice. The combined organic layers were washed with brine (50 mL), dried over Na₂SO₄₍ₛ₎ and filtered. The filtrate was concentrated and purified by C18 column (acetonitrile:water=5% to 100%) to give Int A (1.87 g, 100% purity from LCMS, 94.2% yield) as yellow solids. LC-MS (ESI): R_{T}=2.08 min, mass calcd. for C₃₄H₃₆Cl₂N₄O₃Si 646.2, m/z found 647.1 [M+H]⁺.

Intermediate Int B

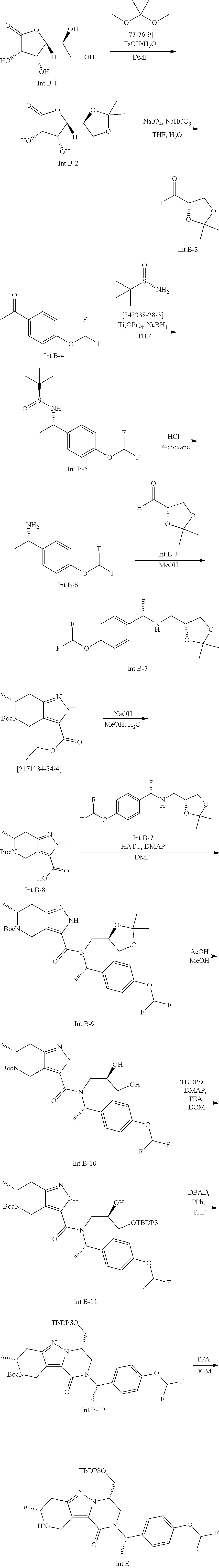

Intermediate Int B-2

(3S,4R,5S)-5-((S)-2,2-Dimethyl-1,3-dioxolan-4-yl)-3,4-dihydroxydihydrofuran-2(3H)-one

To a solution of L-(+)-gulonic acid gamma-lactone Int B-1 (30 g, 168 mmol) in tetrahydrofuran (125 mL) and N,N-dimethylformamide (125 mL) was added 4-methylbenzenesulfonic acid hydrate (3.2 g, 16.8 mmol) and 2,2-dimethoxypropane (23.0 g, 221 mmol) at room temperature. After being stirred at room temperature for 16 hours, sodium carbonate (30.0 g) was added and stirred for 1 hour. The resulting mixture was filtered, and the filter cake was washed with tetrahydrofuran. The filtrate was concentrated under reduced pressure. The resulting brown solids were triturated with toluene (300 mL), filtered, washed with toluene (50 mL), the resulting filter cake was the title product (37.5 g, 85% purity from ¹H NMR, 87% yield) as yellow solids. ¹H NMR (400 MHz, DMSO-d₆) δ 5.88 (d, J=7.2 Hz, 1H), 5.44 (d, J=4.0 Hz, 1H), 4.44-4.41 (m, 1H), 4.30-4.25 (m, 2H), 4.22-4.19 (m, 1H), 4.07 (dd, J=8.8 and 6.4 Hz, 1H), 3.76 (dd, J=8.8 and 6.4 Hz, 1H), 1.35 (s, 3H), 1.29 (s, 3H).

Intermediate Int B-3

(S)-2,2-Dimethyl-1,3-dioxolane-4-carbaldehyde

To a solution of sodium bicarbonate (27.0 g, 321 mmol) in water (150 mL) was added sodium periodate (69.0 g, 323 mmol) and (3S,4R,5S)-5-((S)-2,2-dimethyl-1,3-dioxolan-4-yl)-3,4-dihydroxydihydrofuran-2(3H)-one Int B-2 (36.0 g, 85% purity, 140 mmol) in tetrahydrofuran (160 mL) and water (10 mL) in portions at room temperature. After being stirred for 4.5 h at 35° C., the reaction mixture was cooled to 5° C. and kept at this temperature for 14 h. The solids were removed by filtration and the cake was washed with tetrahydrofuran (100 mL), then the aqueous phase was extracted with tetrahydrofuran (80 mL) twice and ethyl acetate (40 mL) twice. The combined organic layers were dried over Na₂SO₄₍ₛ₎, filtrated, concentrated to give the title product (18.0 g, 70% purity from ¹H NMR, 69% yield) as colorless oil without further purification. ¹H NMR (400 MHz, DMSO-d₆) δ 9.60 (d, J=1.2 Hz, 1H), 4.13-4.06 (m, 1H), 3.93-3.89 (m, 1H), 3.70-3.67 (m, 1H), 1.30 (s, 3H), 1.25 (s, 3H).

Intermediate Int B-5

(S)—N—((S)-1-(4-(Difluoromethoxy)phenyl)ethyl)-2-methylpropane-2-sulfinamide

To a solution of 1-(4-(difluoromethoxy)phenyl)ethanone Int B-4 (25 g, 134 mmol) and (S)-2-methylpropane-2-sulfinamide (33 g, 272 mmol) in tetrahydrofuran (400 mL) was added titanium tetraisopropanolate (83 mL, 280 mmol). After being stirred at 70° C. for 24 hours, the reaction mixture was cooled to 0° C., then sodium borohydride (3.3 g, 87.2 mmol) was added and stirred at 0° C. for 2 hours. The reaction was quenched with brine (40 mL) and filtered with kieselguhr. The cake was washed with ethyl acetate (200 mL). The filtrate was concentrated and purified by silica gel column chromatography (petroleum ether:ethyl acetate=3:1) to give the title compound (20 g, 95% purity from ¹H NMR, 48.6% yield) as yellow oil. LC-MS (ESI): R_{T}=1.60 min, mass calcd. for C₁₃H₁₉F₂NO₂S 291.1, m/z found 292.0 [M+H]⁺. Chiral analysis (Column: Chiralpak IB N-5 5 μm 4.6*250 mm; Mobile Phase: Hex:EtOH=90:10 at 1 mL/min; Temp: 30° C.; Wavelength: 230 nm, R_{T}=5.250 min). ¹H NMR (400 MHz, CDCl₃) δ 7.35 (d, J=8.4 Hz, 2H), 7.10 (d, J=8.4 Hz, 2H), 6.50 (t, J=74.0 Hz, 1H), 4.57-4.52 (m, 1H), 3.38 (br s, 1H), 1.50 (d, J=6.4 Hz, 3H), 1.24 (s, 9H).

Intermediate Int B-6

(S)-1-(4-(Difluoromethoxy)phenyl)ethanamine

To a solution of (S)—N—((S)-1-(4-(difluoromethoxy)phenyl)ethyl)-2-methylpropane-2-sulfinamide Int B-5 (20 g, 95% purity, 65.2 mmol) in methanol (210 mL) was added 4 M hydrochloride in 1,4-dioxane (70 mL) slowly at 0° C. After being stirred at 0° C. for 2 hours, the mixture was concentrated to give the yellow residues, which were dissolved in water (200 mL), extracted with ethyl acetate (40 mL) for three times. The aqueous layer was alkalified with saturated sodium carbonate aqueous solution to pH˜8, extracted with ethyl acetate (300 mL) for five times. The combined organic layers were washed with brine, dried over Na₂SO₄₍ₛ₎ and filtered. The filtrate was concentrated to give the title compound (10.5 g, 90% purity from ¹H NMR, 73.5% yield) as yellow oil. Chiral HPLC (Column: Chiralpak IB N-5 5 μm 4.6*250 mm; Mobile Phase: Hex:EtOH:DEA=85:15:0.2 at 1 mL/min; Temp: 30° C.; Wavelength: 254 nm, R_{T}=7.084 min). ¹H NMR (400 MHz, CDCl₃) δ 7.35 (d, J=8.4 Hz, 2H), 7.08 (d, J=8.4 Hz, 2H), 6.49 (t, J=74.0 Hz, 1H), 4.13 (q, J=6.4 Hz, 1H), 1.79-1.72 (m, 2H), 1.38 (d, J=6.4 Hz, 3H).

Intermediate Int B-7

(S)-1-(4-(Difluoromethoxy)phenyl)-N—(((R)-2,2-dimethyl-1,3-dioxolan-4-yl)methyl)ethanamine

To a mixture of (S)-1-(4-(difluoromethoxy)phenyl)ethanamine Int B-6 (10.0 g, 90% purity, 48.1 mmol) in methanol (50 mL) was added (S)-2,2-dimethyl-1,3-dioxolane-4-carbaldehyde Int B-3 (16.1 g, 70% purity, 86.6 mmol) in tetrahydrofuran (50 mL) and acetic acid (4 mL). After being stirred at room temperature for 10 minutes under nitrogen atmosphere, sodium cyanotrihydroborate (7.6 g, 121 mmol) was added and the mixture was stirred at room temperature for 1 hour under nitrogen atmosphere. Then water (200 mL) was added, and extracted with ethyl acetate (150 mL) for three times. The combined organic layers were concentrated under reduced pressure. The resulting residue was purified by C18 (acetonitrile:water (+0.02% ammonium acetate)=10% to 75%) to give the title product (10.0 g, 100% purity, 69% yield, 97.9% stereopure) as colorless oil. LC-MS (ESI): R_{T}=1.64 min, mass calcd. for C₁₅H₂₁F₂NO₃ 301.2, m/z found 302.0 [M+H]⁺. Chiral analysis (Column: Chiralpak IE 5 μm 4.6*250 mm; Mobile Phase: Hex:EtOH=98:2 at 1 mL/min; Temp: 30° C.; Wavelength: 214 nm, R_{T}=6.421 min).

Intermediate Int B-8

(R)-5-(tert-Butoxycarbonyl)-6-methyl-4,5,6,7-tetrahydro-2H-pyrazolo[4,3-c]pyridine-3-carboxylic acid

To the solution of (R)-5-tert-butyl 3-ethyl 6-methyl-6,7-dihydro-2H-pyrazolo[4,3-c]pyridine-3,5(4H)-dicarboxylate (20 g, 90% purity, 58.2 mmol) in methanol (210 mL) was added sodium hydroxide (7.2 g, 180 mmol) in water (70 mL) at 0° C. After being stirred at 40° C. for 12 hours, water (80 mL) was added into the reaction mixture and concentrated to give the yellow residue, which was added into 1 M hydrochloride aqueous solution until a large amount of solids was precipitated. After filtration, the cake was washed with water (200 mL) and dried under vacuum drying oven at 50° C. for 3 hours, then cooled to room temperature to give the title compound (17.2 g, 95% purity from ¹H NMR, 99.8% yield) as white solids. LC-MS (ESI): R_{T}=1.16 min, mass calcd. for C₁₃H₁₉N₃O₄ 281.1, m/z found 282.1 [M+H]⁺. ¹H NMR (400 MHz, DMSO-d₆) δ 13.14 (s, 2H), 4.89-4.85 (m, 1H), 4.69 (s, 1H), 4.03-3.99 (m, 1H), 2.83 (dd, J=15.6, 5.6 Hz, 1H), 2.54 (d, J=8.0 Hz, 1H), 1.42 (s, 9H), 1.02 (d, J=7.2 Hz, 3H).

Intermediate Int B-9

(R)-tert-Butyl 3-(((S)-1-(4-(difluoromethoxy)phenyl)ethyl)(((R)-2,2-dimethyl-1,3-dioxolan-4-yl)methyl)carbamoyl)-6-methyl-6,7-dihydro-2H-pyrazolo[4,3-c]pyridine-5(4H)-carboxylate

To the solution of (S)-1-(4-(difluoromethoxy)phenyl)-N—(((R)-2,2-dimethyl-1,3-dioxolan-4-yl)methyl)ethanamine Int B-7 (10.5 g, 95% purity, 33.1 mmol) in N,N-dimethylformamide (350 mL) was added (R)-5-(tert-butoxycarbonyl)-6-methyl-4,5,6,7-tetrahydro-2H-pyrazolo[4,3-c]pyridine-3-carboxylic acid Int B-8 (11.7 g, 95% purity, 39.5 mmol), N,N-dimethylpyridin-4-amine (12 g, 98.2 mmol), HATU (19 g, 50.0 mmol) at room temperature. After being stirred at 55° C. for 12 hours, the mixture was diluted with water (1.2 L) and extracted with ethyl acetate (900 mL) twice. The combined organic layers were washed with brine (600 mL), dried over Na₂SO₄₍ₛ₎ and filtered. The filtrate was concentrated and purified by silica gel column chromatography (petroleum ether:ethyl acetate=1:1) to give the title compound (13 g, 95% purity from ¹H NMR, 66.1% yield) as yellow solids. LC-MS (ESI): R_{T}=1.70 min, mass calcd. for C₂₈H₃₈F₂N₄O₆ 564.3, m/z found 565.3 [M+H]⁺. ¹H NMR (400 MHz, CDCl₃) δ 7.45-7.43 (m, 2H), 7.11-7.08 (m, 2H), 6.50 (t, J=73.6 Hz, 1H), 5.02-4.88 (m, 2H), 4.19-4.11 (m, 2H), 4.00-3.98 (m, 1H), 3.58-3.40 (m, 4H), 2.99-2.94 (m, 1H), 2.55 (d, J=16.0 Hz, 1H), 1.73-1.60 (m, 3H), 1.48 (s, 9H), 1.39-1.24 (m, 6H), 1.13 (d, J=6.8 Hz, 3H).

Intermediate Int B-10

(R)-tert-Butyl 3-(((S)-1-(4-(difluoromethoxy)phenyl)ethyl)((R)-2,3-dihydroxypropyl)carbamoyl)-6-methyl-6,7-dihydro-2H-pyrazolo[4,3-c]pyridine-5(4H)-carboxylate

To the solution of (R)-tert-butyl 3-(((S)-1-(4-(difluoromethoxy)phenyl)ethyl)(((R)-2,2-dimethyl-1,3-dioxolan-4-yl)methyl)carbamoyl)-6-methyl-6,7-dihydro-2H-pyrazolo[4,3-c]pyridine-5(4H)-carboxylate Int B-9 (11 g, 95% purity, 18.5 mmol) in methanol (220 mL) and acetic acid (110 mL) at room temperature. After being stirred at 60° C. for 16 hours, the mixture was concentrated and adjust to pH˜8 with saturated sodium bicarbonate aqueous solution, extracted with ethyl acetate (100 mL) twice. The combined organic layers were washed with brine (80 mL) and dried over Na₂SO₄₍ₛ₎ and filtered. The filtrate was concentrated to give the title compound (11 g, 78% purity, 88.4% yield) as yellow oil. LC-MS (ESI): R_{T}=1.50 min, mass calcd. for C₂₅H₃₄F₂N₄O₆ 524.2, m/z found 525.2 [M+H]⁺.

Intermediate Int B-11

(R)-tert-Butyl 3-(((R)-3-((tert-butyldiphenylsilyl)oxy)-2-hydroxypropyl)((S)-1-(4-(difluoromethoxy)phenyl)ethyl)carbamoyl)-6-methyl-6,7-dihydro-2H-pyrazolo[4,3-c]pyridine-5(4H)-carboxylate

To the solution of (R)-tert-butyl 3-(((S)-1-(4-(difluoromethoxy)phenyl)ethyl)((R)-2,3-dihydroxypropyl)carbamoyl)-6-methyl-6,7-dihydro-2H-pyrazolo[4,3-c]pyridine-5(4H)-carboxylate Int B-10 (11 g, 78% purity, 16.4 mmol) in anhydrous dichloromethane (150 mL) was added triethylamine (4.8 mL, 34.4 mmol), N,N-dimethylpyridin-4-amine (200 mg, 1.64 mmol) and tert-butylchlorodiphenylsilane (5.4 g, 19.6 mmol) at 0° C. After being stirred at room temperature for 16 hours, the mixture was diluted with dichloromethane (150 mL) and washed with water (50 mL) twice and brine (50 mL), dried over Na₂SO₄₍ₛ₎ and filtered. The filtrate was concentrated and purified by silica gel column chromatography (petroleum ether:ethyl acetate=1:1) to give the title compound (8.1 g, 100% purity from LCMS, 64.9% yield) as white solids. LC-MS (ESI): R_{T}=1.85 min, mass calcd. for C₄₁H₅₂F₂N₄O₆Si 762.4, m/z found 762.9 [M+H]⁺.

Intermediate Int B-12

(3R,7S)-tert-Butyl 7-(((tert-butyldiphenylsilyl)oxy)methyl)-9-((S)-1-(4-(difluoromethoxy)phenyl)ethyl)-3-methyl-10-oxo-3,4,7,8,9,10-hexahydropyrido[4′,3′:3,4]pyrazolo[1,5-a]pyrazine-2(1H)-carboxylate

To the solution of (R)-tert-butyl 3-(((R)-3-((tert-butyldiphenylsilyl)oxy)-2-hydroxypropyl)((S)-1-(4-(difluoromethoxy)phenyl)ethyl)carbamoyl)-6-methyl-6,7-dihydro-2H-pyrazolo[4,3-c]pyridine-5(4H)-carboxylate Int B-11 (8.1 g, 100% purity, 10.6 mmol) in tetrahydrofuran (160 mL) was added triphenylphosphine (5.5 g, 21.0 mmol) and di-tert-butyl diazene-1,2-dicarboxylate (5.2 g, 22.6 mmol) at 0° C. After being stirred at 0° C. for 2 hours, the mixture was concentrated under reduced pressure to give crude and purified by silica gel column chromatography (petroleum ether:ethyl acetate=2:1) to give the title compound (8.2 g, 95% purity, 98.5% yield) as white solids. LC-MS (ESI): R_{T}=2.07 min, mass calcd. for C₄¹H₅₀F₂N₄O₅Si 744.4, m/z found 744.9 [M+H]⁺. ¹H NMR (400 MHz, CDCl₃) δ 7.61-7.56 (m, 4H), 7.47-7.34 (m, 8H), 7.13-7.11 (m, 2H), 6.50 (t, J=73.6 Hz, 1H), 6.07 (q, J=6.8 Hz, 1H), 5.17-5.12 (m, 1H), 4.90-4.80 (m, 1H), 4.34-4.29 (m, 1H), 4.22-4.18 (m, 1H), 4.10-4.07 (m, 1H), 3.80 (t, J=9.6 Hz, 1H), 3.62 (dd, J=13.2 and 4.8 Hz, 1H), 3.34 (dd, J=13.2 and 4.4 Hz, 1H), 2.90 (dd, J=15.6 and 5.6 Hz, 1H), 3.62 (d, J=15.6 Hz, 1H), 1.53 (d, J=7.2 Hz, 3H), 1.48 (s, 9H), 1.07 (d, J=6.8 Hz, 3H), 1.03 (s, 9H).

Intermediate Int B

(3R,7S)-7-(((tert-Butyldiphenylsilyl)oxy)methyl)-9-((S)-1-(4-(difluoromethoxy)phenyl)ethyl)-3-methyl-1,2,3,4,8,9-hexahydropyrido[4′,3′:3,4]pyrazolo[1,5-a]pyrazin-10(7H)-one

To the solution of (3R,7S)-tert-butyl 7-(((tert-butyldiphenylsilyl)oxy)methyl)-9-((S)-1-(4-(difluoromethoxy)phenyl)ethyl)-3-methyl-10-oxo-3,4,7,8,9,10-hexahydropyrido[4′,3′:3,4]pyrazolo[1,5-a]pyrazine-2(1H)-carboxylate Int B-12 (15.6 g, 95% purity, 19.9 mmol) in dichloromethane (120 mL) was added trifluoroacetic acid (30 mL) at 0° C. After being stirred at 0° C. for 3 hours, the mixture was concentrated and adjusted to pH˜8 with saturated sodium bicarbonate aqueous solution, extracted with ethyl acetate (150 mL) twice. The combined organic layers were washed with brine (100 mL) and dried over Na₂SO₄₍ₛ₎ and filtered. The filtrate was concentrated to give the title compound (9.7 g, 100% purity from LCMS, 75.6% yield) as yellow solids. LC-MS (ESI): R_{T}=1.99 min, mass calcd. for C₃₆H₄₂F₂N₄O₃Si 644.3, m/z found 645.2 [M+H]⁺.

Compounds 1A and 1B

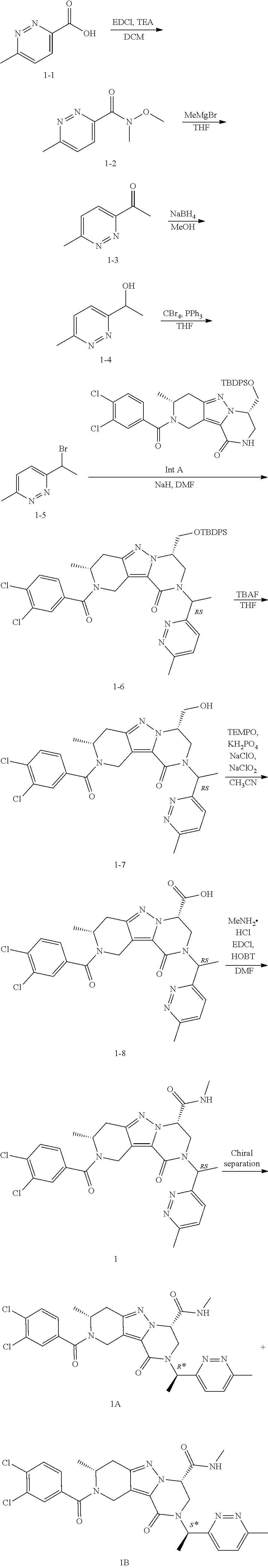

Intermediate 1-2

N-Methoxy-N,6-dimethylpyridazine-3-carboxamide

To a solution of 6-Methylpyridazine-3-carboxylic acid (3.5 g, 25.340 mmol), N,O-dimethylhydroxylamine hydrochloride 1-1 (3.7 g, 37.932 mmol), 1H-benzo[d][1,2,3]triazol-1-ol (4.8 g, 35.523 mmol) and triethylamine (5.3 g, 52.377 mmol) in dichloromethane (20 mL) was added 1-ethyl-3-(3-dimethylaminopropyl)carbodiimide (7.3 g, 38.080 mmol) at room temperature under nitrogen atmosphere. After being stirred at room temperature overnight, the mixture was concentrated and purified by column chromatography on silica gel (petroleum ether:ethyl acetate=1:1) to give the title compound (3 g, 90% purity from ¹H NMR, 59% yield) as a yellow oil. ¹H NMR (400 MHz, CDCl₃) δ 7.85-7.73 (m, 1H), 7.47-7.40 (m, 1H), 3.79 (s, 3H), 3.40 (br s, 3H), 2.78 (s, 3H).

Intermediate 1-3

1-(6-Methylpyridazin-3-yl)ethanone

To the solution of N-methoxy-N,6-dimethylpyridazine-3-carboxamide 1-2 (3 g, 90% purity, 14.901 mmol) in tetrahydrofuran (20 mL) was added 1 M methylmagnesium bromide in tetrahydrofuran (19 mL, 19 mmol) at 0° C. The mixture was stirred at 0° C. for 1 hour. The mixture was quenched with ammonium chloride aqueous solution (10 mL) and extracted with ethyl acetate (10 mL) twice. The combined organic layers were washed with brine (10 mL), dried over Na₂SO₄₍ₛ₎ and filtered. The filtrate was concentrated to give the title compound (2 g, 90% purity from ¹H NMR, 89% yield) as a yellow oil. ¹H NMR (400 MHz, CDCl₃) δ 8.01 (d, J=8.8 Hz, 1H), 7.46 (d, J=8.8 Hz, 1H), 2.86 (s, 3H), 2.80 (s, 3H).

Intermediate 1-4

1-(6-Methylpyridazin-3-yl)ethanol

To a solution of 1-(6-Methylpyridazin-3-yl)ethanone 1-3 (2 g, 90% purity, 13.221 mmol) in methanol (15 mL) was added sodium borohydride (750 mg, 19.824 mmol) at 0° C. After being stirred at 0° C. for 1 hour, the mixture was diluted with 0.5 M hydrochloric acid aqueous solution (10 mL) and extracted with ethyl acetate (10 mL) three times. The combined organic layers were washed with brine (10 mL), dried over Na₂SO₄₍ₛ₎ and filtered. The filtrate was concentrated and purified by silica gel column chromatography (petroleum ether:ethyl acetate=1:1) to give the title compound (1.9 g, 90% purity from ¹H NMR, 94% yield) as a yellow oil. ¹H NMR (400 MHz, CDCl₃) δ 7.44 (d, J=8.8 Hz, 1H), 7.32 (d, J=8.4 Hz, 1H), 5.09 (q, J=6.8 Hz, 1H), 2.69 (s, 3H), 1.54 (d, J=6.8 Hz, 3H).

Intermediate 1-5

3-(1-Bromoethyl)-6-methylpyridazine

To a solution of 1-(6-Methylpyridazin-3-yl)ethanol 1-4 (1.9 g, 90% purity, 12.376 mmol) in tetrahydrofuran (20 mL) were added triphenylphosphine (5 g, 19.063 mmol) and perbromomethane (5 g, 15.077 mmol) at 0° C. After being stirred at 25° C. for 12 hours, the mixture was filtered. The filtrate was concentrated and purified by column chromatography on silica gel (petroleum ether:ethyl acetate=1:1) to give the title compound (1.5 g, 90% purity from ¹H NMR, 54% yield) as a yellow oil. ¹H NMR (400 MHz, CDCl₃) δ 7.64 (d, J=8.8 Hz, 1H), 7.38 (d, J=8.0 Hz, 1H), 5.46 (q, J=6.8 Hz, 1H), 2.74 (s, 3H), 2.11 (d, J=6.8 Hz, 3H).

Intermediate 1-6

(3R,7S)-7-(((tert-Butyldiphenylsilyl)oxy)methyl)-2-(3,4-dichlorobenzoyl)-3-methyl-9-(1-(6-methylpyridazin-3-yl)ethyl)-1,2,3,4,8,9-hexahydropyrido[4′,3′:3,4]pyrazolo[1,5-a]pyrazin-10(7H)-one

To a solution of (3R,7S)-7-(((tert-Butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (900 mg, 87% purity, 1.21 mmol) in N,N-dimethylformamide (6 mL) was added sodium hydride (97 mg, purity 60%, 2.43 mmol) at 0° C. After the mixture was stirred at room temperature for 0.5 hour, a solution of 3-(1-Bromoethyl)-6-methylpyridazine 1-5 (675 mg, 90% purity, 3.02 mmol) in N,N-dimethylformamide (2 mL) was added into the reaction mixture. The resulting reaction mixture was stirred at room temperature for 4 hours. The reaction mixture was poured into 10 mL of cold water and extracted with ethyl acetate (10 mL) for three times. The combined organic layers were washed with brine (20 mL), dried over Na₂SO₄ (s), and filtered. The filtrate was concentrated to give the title compound (1.2 g, 46% purity from LCMS, 59% yield) as white solids. LC-MS (ESI): R_{T}=1.748 min, mass calcd. for C₄₁H₄₄Cl₂N₆O₃Si 766.3, m/z found 769.0 [isotope M+H]⁺.

Intermediate 1-7

(3R,7S)-2-(3,4-Dichlorobenzoyl)-7-(hydroxymethyl)-3-methyl-9-(1-(6-methylpyridazin-3-yl)ethyl)-1,2,3,4,8,9-hexahydropyrido[4′,3′:3,4]pyrazolo[1,5-a]pyrazin-10(7H)-one

To a solution of (3R,7S)-7-(((tert-Butyldiphenylsilyl)oxy)methyl)-2-(3,4-dichlorobenzoyl)-3-methyl-9-(1-(6-methylpyridazin-3-yl)ethyl)-1,2,3,4,8,9-hexahydropyrido[4′,3′:3,4]pyrazolo[1,5-a]pyrazin-10(7H)-one 1-6 (1.2 g, 46% purity, 0.719 mmol) in tetrahydrofuran (8 mL) was added 1 M tetrabutylammonium fluoride in tetrahydrofuran (1 mL, 1 mmol) at room temperature. After being stirred at room temperature for 1 hour, the reaction mixture was quenched with water (10 mL) and extracted with ethyl acetate (10 mL) twice. The combined organic layers were washed with brine (20 mL) twice, dried over Na₂SO₄₍ₛ₎ and filtered. The filtrate was concentrated and purified by column chromatography on silica gel (dichloromethane:ethyl acetate=10:1) to give the title compound (410 mg, 88% purity from LCMS, 95% yield) as white solids. LC-MS (ESI): R_{T}=1.43 min, mass calcd. for C₂₅H₂₆Cl₂N₆O₃ 528.1, m/z found 529.4 [M+H]⁺.

Intermediate 1-8

(3R,7S)-2-(3,4-Dichlorobenzoyl)-3-methyl-9-(1-(6-methylpyridazin-3-yl)ethyl)-10-oxo-1,2,3,4,7,8,9,10-octahydropyrido[4′,3′:3,4]pyrazolo[1,5-a]pyrazine-7-carboxylic acid

To a solution of (3R,7S)-2-(3,4-Dichlorobenzoyl)-7-(hydroxymethyl)-3-methyl-9-(1-(6-methylpyridazin-3-yl)ethyl)-1,2,3,4,8,9-hexahydropyrido[4′,3′:3,4]pyrazolo[1,5-a]pyrazin-10(7H)-one 1-7 (410 mg, 88% purity, 0.682 mmol), sodium chlorite (155 mg, 1.371 mmol) and 2,2,6,6-tetramethylpiperidinooxy (220 mg, 1.399 mmol) in acetonitrile (6 mL) was added saturated potassium phosphate monobasic aqueous solution (6 mL) and sodium hypochlorite solution (1 mL, 10% aqueous solution, 1.679 mmol) at 0° C. After being stirred at room temperature overnight, the reaction mixture was quenched with saturated sodium sulfite aqueous solution (10 mL), acidized with 1 N hydrochloride to pH was 4, and extracted with ethyl acetate (10 mL) twice. The combined organic layers were dried over Na₂SO₄₍ₛ₎, and filtered. The filtrate was concentrated and purified by C18 column (acetonitrile:water=40% to 60%) to give the title compound (390 mg, 93% purity from LCMS, 98% yield) as white solids. LC-MS (ESI): R_{T}=1.22 min, mass calcd. for C₂₅H₂₄Cl₂N₆O₄ 542.1, m/z found 543.2 [M+H]⁺.

Compound 1

(3R,7S)-2-(3,4-Dichlorobenzoyl)-N,3-dimethyl-9-(1-(6-methylpyridazin-3-yl)ethyl)-10-oxo-1,2,3,4,7,8,9,10-octahydropyrido[4′,3′:3,4]pyrazolo[1,5-a]pyrazine-7-carboxamide

To the solution of (3R,7S)-2-(3,4-Dichlorobenzoyl)-3-methyl-9-(1-(6-methylpyridazin-3-yl)ethyl)-10-oxo-1,2,3,4,7,8,9,10-octahydropyrido[4′,3′:3,4]pyrazolo[1,5-a]pyrazine-7-carboxylic acid 1-8 (220 mg, 93% purity, 0.377 mmol) in N,N-dimethylformamide (8 mL) was added methanamine hydrochloride (50 mg, 0.741 mmol), benzotriazol-1-ol (100 mg, 0.740 mmol), 1-ethyl-(3-(3-dimethylamino)propyl)-carbodiimide hydrochloride (140 mg, 0.730 mmol) and triethylamine (187 mg, 1.848 mmol) at 0° C. The mixture was stirred at room temperature for 3 hours. The mixture was diluted with water (10 mL), acidized with 0.5 M hydrochloric acid aqueous solution to pH to 5 and extracted with ethyl acetate (10 mL) twice. The combined organic layers were washed with brine (10 mL), dried over Na₂SO₄₍ₛ₎ and filtered. The filtrate was concentrated and purified by C18 column (acetonitrile:water=30% to 60%) to give the title compound (180 mg, 100% purity from LCMS, 86% yield) as yellow solids. LC-MS (ESI): R_{T}=1.38 min, mass calcd. for C₂₆H₂₇Cl₂N₇O₃ 555.2, m/z found 556.5 [M+H]⁺.

Compounds 1A and 1B

(3R,7S)-2-(3,4-Dichlorobenzoyl)-N,3-dimethyl-9-((R*)-1-(6-methylpyridazin-3-yl)ethyl)-10-oxo-1,2,3,4,7,8,9,10-octahydropyrido[4′,3′:3,4]pyrazolo[1,5-a]pyrazine-7-carboxamide (1A), and (3R,7S)-2-(3,4-Dichlorobenzoyl)-N,3-dimethyl-9-((S*)-1-(6-methylpyridazin-3-yl)ethyl)-10-oxo-1,2,3,4,7,8,9,10-octahydropyrido[4′,3′:3,4]pyrazolo[1,5-a]pyrazine-7-carboxamide (1B)

A racemic mixture of (3R,7S)-2-(3,4-dichlorobenzoyl)-N,3-dimethyl-9-(1-(6-methylpyridazin-3-yl)ethyl)-10-oxo-1,2,3,4,7,8,9,10-octahydropyrido[4′,3′:3,4]pyrazolo[1,5-a]pyrazine-7-carboxamide compound 1 (210 mg, 100% purity, 0.409 mmol) was separated by chiral Prep. HPLC (separation condition: Column: Chiralpak IB 5 μm 30*250 mm; Mobile Phase: ACN=100 at 25 mL/min; Temp: 30° C.; Wavelength: 254 nm) to give compound 1A (57.5 mg, 99.0% purity from LCMS, 32% yield, 100% stereopure) and compound 1B (53.4 mg, 99.6% purity from LCMS, 30% yield, 100% stereopure) as white solids.

Compound 1A

LC-MS (ESI): R_{T}=3.229 min, mass calcd. for C₂₆H₂₇Cl₂N₇O₃ 555.2, m/z found 556.2 [M+H]⁺. Chiral analysis (Column: Chiralpak TB 5 μm 4.6*250 mm; Mobile Phase: ACN=100 at 1 mL/min; Temp: 30° C.; Wavelength: 254 nm; R_{T}=6.903 min). ¹H NMR (400 MHz, CDCl₃) δ 7.53-7.50 (m, 2H), 7.33-7.24 (m, 3H), 6.36-6.18 (m, 1H), 5.96 (br s, 1H), 5.67-5.15 (m, 1H), 4.90-4.88 (m, 1H), 4.74-4.28 (m, 2H), 4.15-4.03 (m, 2H), 3.17-2.92 (m, 1H), 2.73-2.67 (m, 7H), 1.76 (d, J=6.8 Hz, 3H), 1.28 (d, J=6.4 Hz, 3H).

Compound 1B

LC-MS (ESI): R_{T}=3.251 min, mass calcd. for C₂₆H₂₇Cl₂N₇O₃ 555.2, m/z found 556.2 [M+H]⁺. Chiral analysis (Column: Chiralpak IB 5 μm 4.6*250 mm; Mobile Phase: ACN=100 at 1 mL/min; Temp: 30° C.; Wavelength: 254 nm; R_{T}=8.563 min). ¹H NMR (400 MHz, CDCl₃) δ 7.51-7.48 (m, 2H), 7.44-7.35 (m, 1H), 7.31-7.29 (m, 1H), 7.25-7.22 (m, 1H), 6.09-5.83 (m, 2H), 5.70-5.22 (m, 1H), 4.93 (s, 1H), 4.74-4.24 (m, 3H), 3.97-3.94 (m, 1H), 3.14-2.95 (m, 1H), 2.81 (d, J=4.4 Hz, 3H), 2.73-2.65 (m, 4H), 1.74 (d, J=6.8 Hz, 3H), 1.28 (d, J=6.4 Hz, 3H).

Compounds 2A and 2B

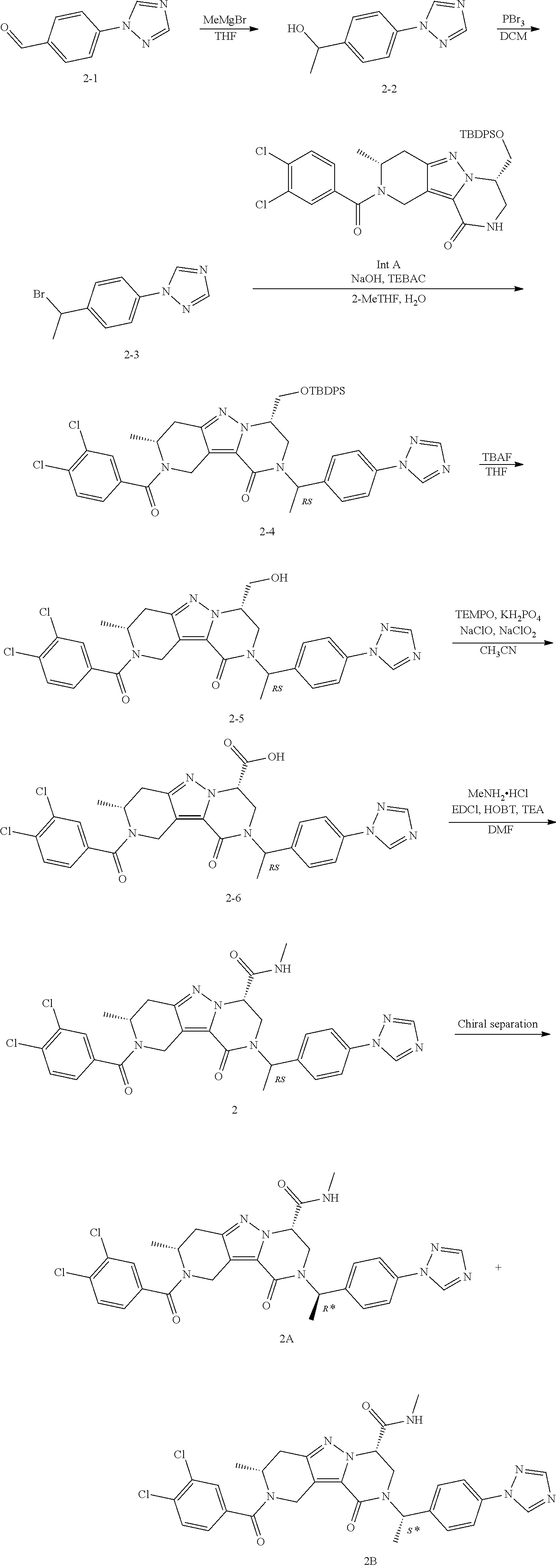

Intermediate 2-2

1-(4-(1H-1,2,4-triazol-1-yl)phenyl)ethanol

To a solution of 4-(1H-1,2,4-triazol-1-yl)benzaldehyde 2-1 (500 mg, 2.89 mmol) in dry tetrahydrofuran (6 mL) was added dropwise 1.0 M methylmagnesium bromide in tetrahydrofuran (4 mL, 4 mmol) at 0° C. under a nitrogen atmosphere. The reaction mixture was stirred at 20° C. for 12 hours. Then, it was quenched with saturated ammonium chloride solution (10 mL), extracted with ethyl acetate (10 mL) for three times, dried over Na₂SO₄₍ₛ₎, and filtered. The filtrate was concentrated to get a residue, which was purified by column chromatography on silica gel (petroleum ether:ethyl acetate=10:1 to 2:1) to give the title product (400 mg, 93% purity from LCMS, 68.1% yield) as white solids. LC-MS (ESI): R_{T}=1.09 min, mass calcd. for C₁₀H₁₁N₃O 189.1 m/z found 190.2 [M+H]⁺. ¹H NMR (400 MHz, CDCl₃) δ 8.54 (s, 1H), 8.10 (s, 1H), 7.66 (d, J=8.4 Hz, 2H), 7.53 (d, J=8.4 Hz, 2H), 4.98 (q, J=6.4 Hz, 1H), 1.6 (s, 1H), 1.54 (d, J=6.0 Hz, 3H).

Intermediate 2-3

1-(4-(l-Bromoethyl)phenyl)-1H-1,2,4-triazole

To a solution of 1-(4-(1H-1,2,4-triazol-1-yl)phenyl)ethanol 2-2 (100 mg, 93% purity, 0.49 mmol) in dichloromethane (2 mL) was added dropwise tribromophosphine (300 mg, 0.37 mmol) in dichloromethane (1 mL) at 0° C. The resulting mixture was stirred at 0° C. for 3 hours. The reaction mixture was concentrated under reduced pressure to give the crude product (540 mg, 71% purity from ¹H NMR, 72.6% yield) as a white solid. The crude was used for next step without purification. LC-MS (ESI): R_{T}=1.65 min, mass calcd. for C₁₀H₁₀BrN₃ 251.0 m/z found 252.0 [M+H]⁺.

Intermediate 2-4

(3R,7S)-9-(1-(4-(1H-1,2,4-Triazol-1-yl)phenyl)ethyl)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (400 mg, 83% purity, 0.51 mmol) and 1-(4-(1-bromoethyl)phenyl)-1H-1,2,4-triazole 2-3 (380 mg, 65% purity, 0.98 mmol) in 2-methyltetrahydrofuran (4 mL) were added sodium hydroxide (4 mL, 50% wt.) and N-benzyl-N,N-diethylethanaminium chloride (10 mg, 0.04 mmol) at 25° C. The resulting mixture was stirred at 50° C. for 2 hours. The reaction mixture was poured into water (10 mL), and extracted with ethyl acetate (10 mL) for three times. The combined organic layers were washed with brine (10 mL), dried over Na₂SO₄₍ₛ₎, and filtered. The filtrate was and concentrated in vacuum to give the crude product (600 mg, 61% purity from LCMS, 87.2% yield) as a yellow oil. The crude used for next step without purification. LC-MS (ESI): R_{T}=1.50 min and 2.11 min, mass calcd. for C₄₄H₄₅N₂C₁₇O₃Si 817.3 m/z found 580.3 [M+H−TBDPS]⁻ and 818.5 [M+H]⁺.

Intermediate 2-5

(3R,7S)-9-(1-(4-(1H-1,2,4-Triazol-1-yl)phenyl)ethyl)-2-(3,4-dichlorobenzoyl)-7-(hydroxymethyl)-3-methyl-1,2,3,4,8,9-hexahydropyrido[4′,3′:3,4]pyrazolo[1,5-a]pyrazin-10(7H)-one

To a solution of (3R,7S)-9-(1-(4-(1H-1,2,4-triazol-1-yl)phenyl)ethyl)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one 2-4 (600 mg, 61% purity, 0.34 mmol) in tetrahydrofuran (6 mL) was added 1.0 M tetrabutylammonium fluoride solution in tetrahydrofuran (0.5 mL, 0.5 mmol) at 0° C. Then the reaction mixture was stirred at 20° C. for 2 hours. The reaction mixture was poured into water (10 mL), and extracted with ethyl acetate (10 mL) for three times. The combined organic layers were washed with brine (10 mL), dried over Na₂SO₄₍ₛ₎, and filtered. The filtrate was and concentrated in vacuum, which was purified by C18 chromatography (acetonitrile:water (+0.02% ammonium bicarbonate)=40-60%) to give the title product (160 mg, 90% purity from ¹H NMR, 55.5% yield) as a colorless solid. LC-MS (ESI): R_{T}=2.44 min, mass calcd. for C₂₈H₂₇Cl₂N₇O₃ 579.2 m/z found 580.1 [M+H]⁺. ¹H NMR (300 MHz, CDCl₃) δ 8.61 (s, 1H), 8.15 (s, 1H), 7.82-7.69 (m, 2H), 7.66 (d, J=6.3 Hz, 1H), 7.59-7.49 (m, 3H), 7.33-7.30 (m, 1H), 6.28-6.06 (m, 1H), 5.93-5.33 (m, 1H), 5.29-4.77 (m, 1H), 4.74-4.40 (m, 2H), 4.37-4.25 (m, 1H), 4.10-3.98 (m, 1H), 3.92-3.71 (m, 1H), 3.69-3.55 (m, 1H), 3.40-3.21 (m, 1H), 3.18-2.97 (m, 1H), 2.72-2.50 (m, 1H), 1.68 (d, J=7.2 Hz, 3H), 1.33-1.28 (m, 3H).

Intermediate 2-6

(3R,7S)-9-(1-(4-(1H-1,2,4-Triazol-1-yl)phenyl)ethyl)-2-(3,4-dichlorobenzoyl)-3-methyl-10-oxo-1,2,3,4,7,8,9,10-octahydropyrido[4′,3′:3,4]pyrazolo[1,5-a]pyrazine-7-carboxylic acid

To a solution of (3R,7S)-9-(1-(4-(1H-1,2,4-triazol-1-yl)phenyl)ethyl)-2-(3,4-dichlorobenzoyl)-7-(hydroxymethyl)-3-methyl-1,2,3,4,8,9-hexahydropyrido[4′,3′:3,4]pyrazolo[1,5-a]pyrazin-10(7H)-one 2-5 (160 mg, 90% purity, 0.25 mmol) in acetonitrile (1.4 mL) were added saturated potassium dihydrogen phosphate (1.4 mL), 2,2,6,6-tetramethylpiperidinooxy (80 mg, 0.51 mmol), sodium chlorite (60 mg, 0.53 mmol) and dropwise 10% sodium hypochlorite solution (370 mg, 0.34 mmol) at 0° C. The reaction mixture was allowed to slowly return to room temperature. After being stirred at room temperature for 14 hours, the reaction mixture was quenched with saturated sodium thiosulfate aqueous solution (2 mL), acidized with 1.0 M hydrochloric acid solution to pH=4˜5, and extracted with ethyl acetate (10 mL) for three times. The combined organic layers were washed with brine (10 mL), dried over Na₂SO₄₍ₛ₎, and filtered. The filtrate was concentrated to get a residue. The residue was triturated with (petroleum ether:dichloromethane=10:1). The resulting solid was filtered through a sand funnel, rinsed with petroleum ether (3×5 mL), and collected to give the title product (160 mg, 92% purity from LCMS, 99.8% yield) as white solids. LC-MS (ESI): R_{T}=1.25 min, mass calcd. for C₂₈H₂₅Cl₂N₇O₄ 593.1 m/z found 594.2 [M+H]⁺.

Compound 2

(3R,7S)-9-(1-(4-(1H-1,2,4-Triazol-1-yl)phenyl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide

To a solution of (3R,7S)-9-(1-(4-(1H-1,2,4-triazol-1-yl)phenyl)ethyl)-2-(3,4-dichlorobenzoyl)-3-methyl-10-oxo-1,2,3,4,7,8,9,10-octahydropyrido[4′,3′:3,4]pyrazolo[1,5-a]pyrazine-7-carboxylic acid 2-6 (160 mg, 90% purity, 0.24 mmol), methylamine hydrochloride (50 mg, 0.74 mmol), N1-((ethylimino)methylene)-N3,N3-dimethylpropane-1,3-diamine hydrochloride (95 mg, 0.5 mmol) and 1H-benzo[d][1,2,3]triazol-1-ol (68 mg, 0.5 mmol) in N,N-dimethylformamide (1.5 mL) was added dropwise triethylamine (160 mg, 1.58 mmol) in N,N-dimethylformamide (0.5 mL) at 0° C. Then the reaction mixture was stirred at 20° C. for 2 hours. The reaction mixture was poured into water (10 mL), and extracted with ethyl acetate (10 mL) for three times. The combined organic layers were washed with brine (10 mL), dried over Na₂SO₄₍ₛ₎, and filtered. The filtrate was and concentrated in vacuum to give a residue, which was purified by C18 chromatography (acetonitrile:water (+0.02% ammonium bicarbonate)=40-60%) to give the title (80 mg, 94% purity from LCMS, 51.1% yield) as a colorless solid. LC-MS (ESI): R_{T}=1.46 min, mass calcd. for C₂₉H₂₈Cl₂N₈O₃ 606.2 m/z found 607.2 [M+H]⁺. H NMR (300 MHz, CDCl₃) δ 8.60 (d, J=3.3 Hz, 1H), 8.15 (d, J=2.4 Hz, 1H), 7.74-7.70 (m, 2H), 7.58-7.56 (m, 3H), 7.51-7.48 (m, 1H), 7.33-7.32 (m, 1H), 6.24-5.99 (m, 2H), 5.82-5.24 (m, 1H), 5.50-4.81 (m, 1H), 4.73-4.31 (m, 1H), 4.02-3.89 (m, 1H), 3.47 (dd, J=13.5, 4.8 Hz, 1H), 3.24 (br s, 1H), 2.85 (d, J=4.5 Hz, 2H), 2.79-2.68 (m, 3H), 1.69-1.66 (m, 3H), 1.36-1.31 (m, 3H).

Compounds 2A and 2B

(3R,7S)-9-((R*)-1-(4-(1H-1,2,4-triazol-1-yl)phenyl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide (2A), and (3R,7S)-9-((S*)-1-(4-(1H-1,2,4-triazol-1-yl)phenyl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide (2B)

The racemic mixture of (3R,7S)-9-(1-(4-(1H-1,2,4-triazol-1-yl)phenyl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide compound 2 (130 mg, 94% purity, 0.2 mmol) was separated by chiral prep HPLC (separation condition: Column: Chiralpak IE 5 μm 20*250 mm; Mobile Phase: MeOH:EtOH=50:50 at 25 mL/min; Temp: 30° C.; Wavelength: 254 nm) to give compound 2A (32.3 mg, 99.6% purity, 26.3% yield, 99.8% stereopure) and compound 2B (66.7 mg, 98.6% purity, 52.2% yield, 100% stereopure) as white solids.

Compound 2A

LC-MS (ESI): R_{T}=3.427 min, mass calcd. for C₂₉H₂₈Cl₂N₈O₃ 606.2 m/z found 607.2 [M+H]⁺. Chiral analysis: (Column: Chiralpak IE 5 μm 4.6*250 mm; Mobile Phase: MeOH:EtOH=50:50 at 1 mL/min; Temp: 30° C.; Wavelength: 254 nm, R_{T}=8.411 min). ¹H NMR (400 MHz, DMSO-d₆) δ 9.32 (s, 1H), 8.24 (s, 1H), 7.85 (d, J=8.4 Hz, 2H), 7.79-7.74 (m, 3H), 7.52-7.32 (m, 3H), 5.91-5.66 (m, 1H), 5.57-5.13 (m, 1H), 4.98 (s, 1H), 4.70-4.40 (m, 1H), 4.32-4.13 (m, 1H), 4.09-3.94 (m, 1H), 3.44-3.33 (m, 1H), 2.97-2.84 (m, 1H), 2.60-2.52 (m, 1H), 2.38 (d, J=4.4 Hz, 3H), 1.62-1.42 (m, 3H), 1.29-1.11 (m, 3H).

Compound 2B

LC-MS (ESI): R_{T}=3.416 min, mass calcd. for C₂₉H₂₈Cl₂N₈O₃ 606.2 m/z found 607.3 [M+H]⁺. Chiral analysis: (Column: Chiralpak IE 5 μm 4.6*250 mm; Mobile Phase: MeOH:EtOH=50:50 at 1 mL/min; Temp: 30° C.; Wavelength: 254 nm, R_{T}=11.881 min). ¹H NMR (400 MHz, DMSO-d₆) δ 9.30 (s, 11), 8.23 (s, 1H), 8.08-7.98 (m, 1H), 7.90-7.81 (m, 2H), 7.75 (d, J=8.4 Hz, 2H), 7.60-7.40 (m, 3H), 5.95-5.67 (m, 1H), 5.53-5.17 (m, 1H), 5.11-4.92 (m, 1H), 4.70-4.36 (m, 1H), 4.29-4.10 (m, 1H), 3.78-3.50 (m, 2H), 2.98-2.83 (m, 1H), 2.64 (d, J=4.4 Hz, 3H), 2.58-2.52 (m, 1H), 1.55-1.38 (m, 3H), 1.31-1.11 (m, 3H).

Compounds 3A and 3B

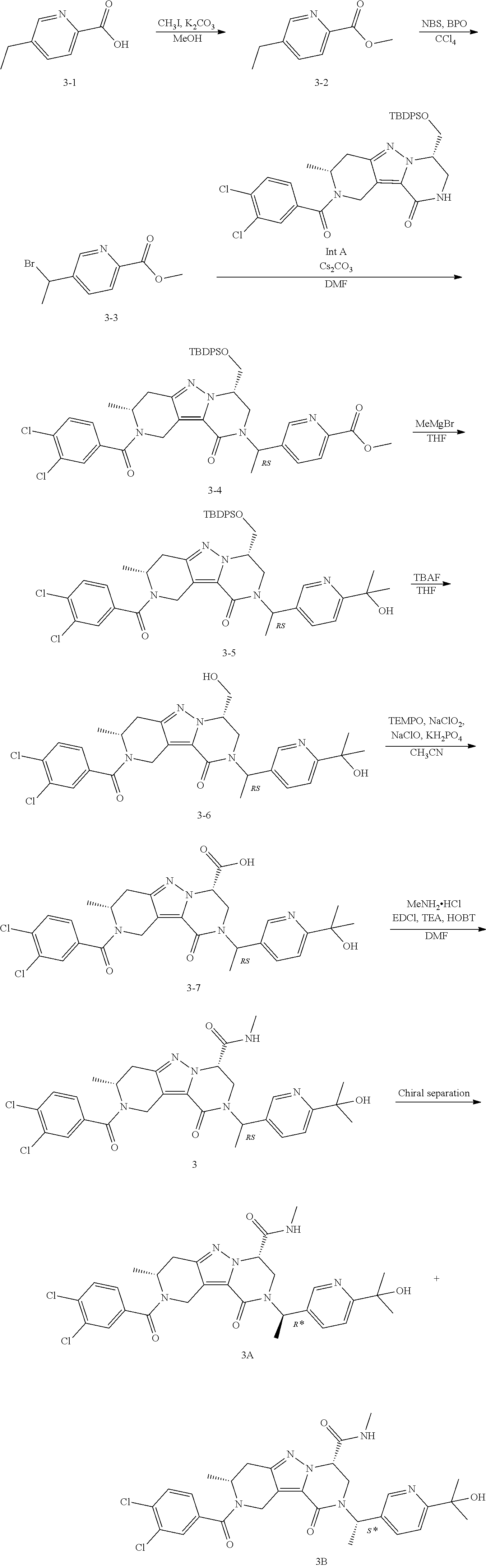

Intermediate 3-2

Methyl 5-ethylpicolinate

To a solution of 5-ethylpicolinic acid 3-1 (500 mg, 3.31 mmol) and potassium carbonate (800 mg, 5.79 mmol) in N, N-dimethylformamide (8 mL) was added iodomethane (800 mg, 5.64 mmol) at 0° C. The reaction mixture was stirred at room temperature for 4 hours, the reaction mixture was quenched with water (20 mL) and extracted with ethyl acetate (20 mL) for three times. The combined organic layers were washed with brine (20 mL), dried over Na₂SO₄₍ₛ₎, and filtered. The filtrate was concentrated in vacuum to give a residue, which was purified by C18 chromatography (acetonitrile:water (+0.02% ammonium bicarbonate)=40-60%) to give the title (280 mg, 100% purity from LCMS, 51.2% yield) as a colorless oil. LC-MS (ESI): R_{T}=1.34 min, mass calcd. for C₉H₁₁NO₂ 165.1 m/z found 166.1 [M+H]⁺.

Intermediate 3-3

Methyl 5-(1-bromoethyl)picolinate

To a solution of methyl 5-ethylpicolinate 3-2 (280 mg, 100% yield, 1.70 mmol) and N-Bromosuccinimide (340 mg, 1.91 mmol) in tetrachloromethane (6 mL) were added dibenzoyl peroxide (50 mg, 0.21 mmol) at 25° C. The resulting mixture was stirred at 80° C. for 2 hours. The reaction mixture was poured into water (10 mL) and extracted with dichloromethane (10 mL) for three times. The combined organic layers were washed with brine (10 mL), dried over Na₂SO₄₍ₛ₎, and filtered. The filtrate was and concentrated in vacuum to give a residue, which was purified by C18 chromatography (acetonitrile:water (+0.02% ammonium bicarbonate)=40-60%) to give the title (380 mg, 100% purity from LCMS, 91.8% yield) as a colorless oil. LC-MS (ESI): R_{T}=1.42 min, mass calcd. for C₉H₁₀BrNO₂ 243.0 m/z found 246.0 [M+H]⁺.

Intermediate 3-4

Methyl 5-(1-((3R,7S)-7-(((tert-butyldiphenylsilyl)oxy)methyl)-2-(3,4-dichlorobenzoyl)-3-methyl-10-oxo-1,2,3,4,7,8-hexahydropyrido[4′,3′:3,4]pyrazolo[1,5-a]pyrazin-9(10H)-yl)ethyl)picolinate

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (750 mg, 90% purity, 1.04 mmol) and methyl 5-(1-bromoethyl)picolinate 3-3 (330 mg, 1.35 mmol) in N,N-dimethylformamide (10 mL) was added cesium carbonate (900 mg, 2.76 mmol) at 20° C. Then the reaction mixture was stirred at 40° C. for 4 hours. The reaction mixture was poured into water (10 mL) and extracted with ethyl acetate (20 mL) for three times. The combined organic layers were washed with brine (20 mL), dried over Na₂SO₄₍ₛ₎, and filtered. The filtrate was and concentrated in vacuum to give a residue, which was purified by C18 chromatography (acetonitrile:water (+0.02% ammonium bicarbonate)=60-90%) to give the title (710 mg, 98.2% purity from LCMS, 82.5% yield) as a white solid. LC-MS (ESI): R_{T}=2.12 min and 2.19 min, mass calcd. for C₄₄H₄₉N₅Cl₂O₄Si 809.3 m/z found 810.6 [M+H]⁺.

Intermediate 3-5

(3R,7S)-7-(((tert-Butyldiphenylsilyl)oxy)methyl)-2-(3,4-dichlorobenzoyl)-9-(1-(6-(2-hydroxypropan-2-yl)pyridin-3-yl)ethyl)-3-methyl-1,2,3,4,8,9-hexahydropyrido[4′,3′:3,4]pyrazolo[1,5-a]pyrazin-10(7H)-one

To a solution of methyl 5-(1-((3R,7S)-7-(((tert-butyldiphenylsilyl)oxy)methyl)-2-(3,4-dichlorobenzoyl)-3-methyl-10-oxo-1,2,3,4,7,8-hexahydropyrido[4′,3′:3,4]pyrazolo[1,5-a]pyrazin-9(10H)-yl)ethyl)picolinate 3-4 (610 mg, 98.2% purity, 0.74 mmol) in tetrahydrofuran (6 mL) was added methylmagnesium bromide (1.0 M in tetrahydrofuran, 3 mL, 3 mmol) at 0° C. under nitrogen. The resulting mixture was allowed to warm to 40° C. and stirred for 13 hours. The reaction mixture was quenched with saturated ammonium chloride solution (10 mL), extracted with ethyl acetate (10 mL) three times, dried over Na₂SO₄₍ₛ₎, and filtered. The filtrate was concentrated to get a residue, which was purified by C18 chromatography (acetonitrile:water (+0.02% ammonium bicarbonate)=70-90%) to give the title product (400 mg, 81% purity from LCMS, 54.1% yield) as a white solid. LC-MS (ESI): R_{T}=1.14 min and 1.19 min, mass calcd. for C₄₄H₄₉N₅Cl₂O₄Si 809.3 m/z found 810.6 [M+H]⁺.

Intermediate 3-6

(3R,7S)-2-(3,4-Dichlorobenzoyl)-7-(hydroxymethyl)-9-(1-(6-(2-hydroxypropan-2-yl)pyridin-3-yl)ethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-9-(1-(6-(2-hydroxypropan-2-yl)pyridin-3-yl)ethyl)-3-methyl-1,2,3,4,8,9-hexahydropyrido[4′,3′:3,4]pyrazolo[1,5-a]pyrazin-10(7H)-one 3-5 (300 mg, 81% purity, 0.3 mmol) in tetrahydrofuran (3 mL) was added 1 M tetrabutylammonium fluoride solution in tetrahydrofuran (0.3 mL, 0.3 mmol) at 0° C. Then the reaction mixture was stirred at 20° C. for 2 hours. The reaction mixture was poured into water (10 mL), and extracted with ethyl acetate (10 mL) three times. The combined organic layers were washed with brine (10 mL), dried over Na₂SO₄₍ₛ₎, and filtered. The filtrate was concentrated in vacuum, which was purified by C18 chromatography (acetonitrile:water (+0.02% ammonium bicarbonate)=40
60%) to give the title product (160 mg, 81% purity from LCMS, 75.5% yield) as a colorless solid. LC-MS (ESI): R_{T}=1.45 min, mass calcd. for C₂₈H₃₁Cl₂N₅O₄ 571.2 m/z found 572.2 [M+H]⁺.

Intermediate 3-7

(3R,7S)-2-(3,4-Dichlorobenzoyl)-9-(1-(6-(2-hydroxypropan-2-yl)pyridin-3-yl)ethyl)-3-methyl-10-oxo-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-7-(hydroxymethyl)-9-(1-(6-(2-hydroxypropan-2-yl)pyridin-3-yl)ethyl)-3-methyl-1,2,3,4,8,9-hexahydropyrido[4′,3′:3,4]pyrazolo[1,5-a]pyrazin-10(7H)-one 3-6 (160 mg, 81% purity, 0.23 mmol) in acetonitrile (1.5 mL) were added saturated potassium dihydrogen phosphate (1.5 mL), 2,2,6,6-tetramethylpiperidinooxy (75 mg, 0.48 mmol), sodium chlorite (45 mg, 0.50 mmol) and dropwise 10% sodium hypochlorite solution (380 mg, 0.51 mmol) at 0° C. The reaction mixture was allowed to slowly return to room temperature. After being stirred at room temperature for 14 hours, the reaction mixture was quenched with saturated sodium thiosulfate aqueous solution (2 mL), acidized with 1.0 M hydrochloric acid solution to pH=4˜5, and extracted with ethyl acetate (10 mL) three times. The combined organic layers were washed with brine (10 mL), dried over Na₂SO₄₍ₛ₎, and filtered. The filtrate was concentrated to get a residue. The residue was purified by C18 chromatography (acetonitrile:water (+0.02% ammonium bicarbonate)=30-50%) to give the title product (130 mg, 100% purity from LCMS, 97.9% yield) as white solids. LC-MS (ESI): R_{T}=1.23 min, mass calcd. for C₂₈H₂₉Cl₂N₅O₅ 585.2 m/z found 586.3 [M+H]⁺. ¹H NMR (300 MHz, CD₃OD) δ 8.64-8.45 (m, 1H), 8.12-7.91 (m, JH), 7.86-7.75 (m, 1H), 7.74-7.67 (m, 2H), 7.47-7.40 (m, 1H), 6.15-5.76 (m, 1H), 5.72-5.32 (m, 1H), 5.26-5.08 (m, 1H), 4.80-4.57 (m, 1H), 4.55-4.27 (m, 1H), 4.10-3.98 (m, 1H), 3.88-3.67 (m, 1H), 3.35-3.23 (m, 1H), 3.15-2.99 (m, 1H), 2.85-2.63 (m, 1H), 1.77-1.65 (m, 3H), 1.62 (s, 3H), 1.58 (s, 3H), 1.43-1.33 (m, 3H).

Compound 3

(3R,7S)-2-(3,4-Dichlorobenzoyl)-9-(1-(6-(2-hydroxypropan-2-yl)pyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide

To a solution of (3R,7S)-2-(3,4-dichlorobenzoyl)-9-(1-(6-(2-hydroxypropan-2-yl)pyridin-3-yl)ethyl)-3-methyl-10-oxo-1,2,3,4,7,8,9,10-octahydropyrido[4′,3′:3,4]pyrazolo[1,5-a]pyrazine-7-carboxylic acid 3-7 (130 mg, 74% purity, 0.22 mmol), methylamine hydrochloride (40 mg, 0.59 mmol), N1-((ethylimino)methylene)-N3,N3-dimethylpropane-1,3-diamine hydrochloride (100 mg, 0.52 mmol) and 1H-benzo[d][1,2,3]triazol-1-ol (80 mg, 0.59 mmol) in N,N-dimethylformamide (1.5 mL) was added dropwise trimethylamine (140 mg, 1.38 mmol) in N,N-dimethylformamide (0.5 mL) at 0° C. Then the reaction mixture was stirred at 20° C. for 2 hours. The reaction mixture was poured into water (10 mL), and extracted with ethyl acetate (10 mL) three times. The combined organic layers were washed with brine (10 mL), dried over Na₂SO₄₍ₛ₎, and filtered. The filtrate was concentrated in vacuum to give a residue, which was purified by C18 chromatography (acetonitrile:water (+0.02% ammonium bicarbonate)=40-60%) to give the title (130 mg, 96% purity from LCMS, 93.9% yield) as a colorless solid. LC-MS (ESI): R_{T}=2.41 min, mass calcd. for C₂₉H₃₂Cl₂N₆O₄ 598.2 m/z found 599.3 [M+H]⁺.

Compounds 3A and 3B

(3R,7S)-2-(3,4-Dichlorobenzoyl)-9-((R*)-1-(6-(2-hydroxypropan-2-yl)pyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide (3A), and (3R,7S)-2-(3,4-Dichlorobenzoyl)-9-((S*)-1-(6-(2-hydroxypropan-2-yl)pyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide (3B)

The racemate (3R,7R)-2-(3,4-dichlorobenzoyl)-9-(1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one compound 3 (130 mg, 96% purity, 0.21 mmol) was separated by chiral prep HPLC (separation condition: Column: Chiralpak IE 5 μm 20*250 mm; Mobile Phase: ACN:IPA:DEA=70:30:0.2 at 25 mL/min; Temp: 30° C.; Wavelength: 254 nm) to give compound 3A (25.8 mg, 98.5% purity, 20.4% yield, 99.7% stereopure) and compound 3B (50 mg, 99.2% purity, 39.7% yield, 99.8% stereopure) all as white solids.

Compound 3A

LC-MS (ESI): R_{T}=3.682 min, mass calcd. for C₂₉H₃₂Cl₂N₆O₄ 598.2 m/z found 599.3 [M+H]⁺. Chiral analysis: (Column: Chiralpak IE 5 μm 4.6*250 mm; Mobile Phase: ACN:IPA:DEA=70:30:0.2 at 1 mL/min; Temp: 30° C.; Wavelength: 254 nm, R_{T}=5.460 min). ¹H NMR (400 MHz, DMSO-d₆) δ 8.36 (s, 1H), 7.78 (dd, J=17.2 and 4.8 Hz, 3H), 7.60 (s, 2H), 7.46 (d, J=7.6 Hz, 111), 5.88-5.63 (m, 1H), 5.49-5.24 (m, 1H), 5.20 (s, 1H), 4.97 (s, 1H), 4.70-4.37 (m, 1H), 4.30-4.13 (m, 1H), 4.09-3.92 (m, 1H), 3.81-3.52 (m, 1H), 2.96-2.84 (m, 1H), 2.65-2.55 (m, 1H), 2.35 (d, J=4.4 Hz, 3H), 1.62-1.45 (m, 3H), 1.42 (s, 6H), 1.30-1.09 (m, 3H).

Compound 3B

LC-MS (ESI): R_{T}=3.761 min, mass calcd. for C₂₉H₃₂Cl₂N₆O₄ 598.2 m/z found 599.2 [M+H]⁺. Chiral analysis: (Column: Chiralpak IE 5 μm 4.6*250 mm; Mobile Phase: ACN:IPA:DEA=70:30:0.2 at 1 mL/min; Temp: 30° C.; Wavelength: 254 nm, R_{T}=7.106 min). ¹H NMR (400 MHz, DMSO-d₆) δ 8.46 (s, 1H), 8.07-7.98 (m, 1H), 7.74 (d, J=8.0 Hz, 3H), 7.60-7.56 (m, 1H), 7.45 (d, J=8.0 Hz, 1H), 5.88-5.60 (m, 1H), 5.51-5.33 (m, 1H), 5.20 (s, 1H), 5.05 (s, 1H), 4.65-4.40 (m, 1H), 4.28-4.10 (m, 1H), 3.83-3.57 (m, 2H), 2.97-2.84 (m, 1H), 2.64 (d, J=4.8 Hz, 3H), 2.51-2.50 (m, 1H), 1.53-1.37 (m, 9H), 1.29-1.10 (m, 3H).

Compounds 4A and 4B (Preparation Method A)

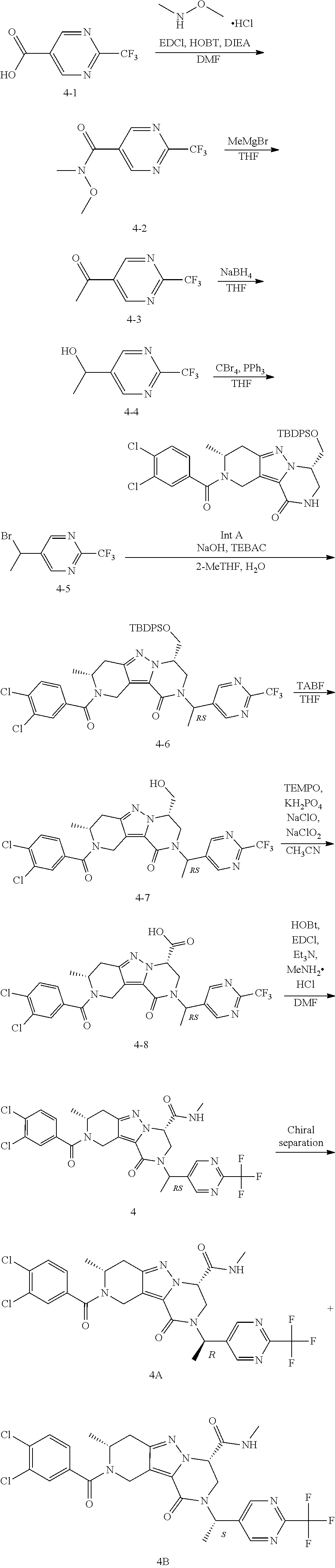

Intermediate 4-2

N-Methoxy-N-methyl-2-(trifluoromethyl)pyrimidine-5-carboxamide

To the solution of 2-(trifluoromethyl)pyrimidine-5-carboxylic acid 4-1 (5.0 g, 26.0 mmol) in N,N-dimethylformamide (55 mL) was added N,O-dimethylhydroxylamine hydrochloride (5.1 g, 52.3 mmol), benzotriazol-1-ol (7.1 g, 52.5 mmol), 1-ethyl-(3-(3-dimethylamino)propyl)-carbodiimide hydrochloride (10.1 g, 52.7 mmol) and N-ethyl-N-isopropylpropan-2-amine (20.5 g, 158.6 mmol) at 0° C. The mixture was stirred at room temperature for 16 hours. The mixture was diluted with water (100 mL), acidized with 0.5 M hydrochloric acid aqueous solution to pH˜5 and extracted with ethyl acetate (100 mL) twice. The combined organic layers were washed with brine (100 mL), dried over Na₂SO₄₍ₛ₎ and filtered. The filtrate was concentrated and purified by C18 column (acetonitrile:water=30% to 60%) to give the title compound (5 g, 100% purity from LCMS, 81.7% yield) as yellow solids. LC-MS (ESI): R_{T}=1.40 min, mass calcd. for C₈H₈F₃N₃O₂ 235.1, m/z found 236.1 [M+H]⁺. ¹H NMR (400 M Hz, CDCl₃) δ 9.24 (s, 2H), 3.62 (s, 3H), 3.44 (s, 3H).

Intermediate 4-3

1-(2-(Trifluoromethyl)pyrimidin-5-yl)ethanone

To a solution of N-Methoxy-N-methyl-2-(trifluoromethyl)pyrimidine-5-carboxamide 4-2 (5 g, 100% purity, 21.3 mmol) in tetrahydrofuran (50 mL) was added 1 M methylmagnesium bromide in 2-methyltetrahydrofuran (33.3 mL, 33.3 mmol) dropwise at 0° C. After being stirred at 0° C. for 2 hours, the reaction mixture was poured into saturated aqueous ammonium chloride solution (50 mL) and extracted with ethyl acetate (50 mL) for twice. The combined organic layers were washed with brine (50 mL), dried over Na₂SO₄₍ₛ₎ and filtered. The filtrate was concentrated to give the title compound (4.5 g, 77% purity from LCMS, 85.7% yield) as a yellow oil. LC-MS (ESI): R_{T}=1.36 min, mass calcd. for C₇H₅F₃N₂O 190.0, m/z found 189.0 [M−H]⁻.

Intermediate 4-4

1-(2-(Trifluoromethyl)pyrimidin-5-yl)ethanol

To the solution of 1-(2-(trifluoromethyl)pyrimidin-5-yl)ethanone 4-3 (4.5 g, 77% purity, 18.2 mmol) in tetrahydrofuran (50 mL) was added sodium borohydride (1.54 g, 40.7 mmol) at 0° C. After being stirred at 0° C. for 1 hour, the mixture was quenched with saturated aqueous ammonium chloride solution (50 mL), which was diluted with ethyl acetate (50 mL), washed with water (50 mL) twice, dried over Na₂SO₄₍ₛ₎ and concentrated under vacuum to give the residue, which was purified by C18 (acetonitrile:water=40% to 65%) to give the desired product (2.3 g, 90% purity, 59.1% yield) as a yellow oil. ¹H NMR (400 M Hz, CDCl₃) δ 8.92 (s, 2H), 5.15-5.06 (m, 1H), 1.62-1.61 (m, 3H).

Intermediate 4-5

5-(1-bromoethyl)-2-(trifluoromethyl)pyrimidine

To a solution of 1-(2-(trifluoromethyl)pyrimidin-5-yl)ethanol 4-4 (2 g, 90% purity, 9.37 mmol) in tetrahydrofuran (20 mL) were added triphenylphosphine (5 g, 19.1 mmol) and perbromomethane (5 g, 15.1 mmol) at 0° C. After being stirred at 25° C. for 0.5 hour, the mixture was filtered. The filtrate was concentrated under reduced pressure to give the residue, which was purified by silica gel column chromatography (petroleum ether:ethyl acetate=100:1) to give the title compound (1 g, 90% purity from ¹H NMR, 37.7% yield) as a yellow oil. ¹H NMR (400 M Hz, CDCl₃) δ 8.96 (s, 2H), 5.18-5.17 (m, 1H), 2.13-2.11 (m, 3H).

Intermediate 4-6

(3R,7S)-7-(((tert-Butyldiphenylsilyl)oxy)methyl)-2-(3,4-dichlorobenzoyl)-3-methyl-9-(1-(2-(trifluoromethyl)pyrimidin-5-yl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (1 g, 100% purity, 1.54 mmol), N-benzyl-N,N-diethylethanaminium chloride (2 mg, 0.009 mmol) and 5-(1-bromoethyl)-2-(trifluoromethyl)pyrimidine 4-5 (1 g, 3.53 mmol) in 2-methyltetrahydrofuran (12 mL) was added 50% wt. sodium hydroxide in water (12 mL) slowly at 30° C. After being stirred at 30° C. for 2 hours, the reaction mixture was diluted with water (15 mL) and extracted with ethyl acetate (15 mL) twice. The combined organic layers were washed with brine (15 mL) twice, dried over Na₂SO₄₍ₛ₎ and filtered. The filtrate was concentrated under reduced pressure to give the title compound (1.2 g, 73% purity, 69% yield) as a yellow oil. LC-MS (ESI): R_{T}=1.94 min, mass calcd. for C₄¹H₄₁Cl₂F₃N₆O₃Si 820.2, m/z found 821.5 [M+H]⁺.

Intermediate 4-7

(3R,7S)-2-(3,4-Dichlorobenzoyl)-7-(hydroxymethyl)-3-methyl-9-(1-(2-(trifluoromethyl)pyrimidin-5-yl)ethyl)-1,2,3,4,8,9-hexahydropyrido[4′,3′:3,4]pyrazolo[1,5-a]pyrazin-10(7H)-one

To the solution of (3R,7S)-7-(((tert-butyldiphenylsilyl)oxy)methyl)-2-(3,4-dichlorobenzoyl)-3-methyl-9-(1-(2-(trifluoromethyl)pyrimidin-5-yl)ethyl)-1,2,3,4,8,9-hexahydropyrido[4′,3′:3,4]pyrazolo[1,5-a]pyrazin-10(7H)-one 4-6 (1.2 g, 73% purity, 1.07 mmol) in tetrahydrofuran (12 mL) was added 1 M tetrabutylammonium fluoride solution (1.6 mL). After being stirred at room temperature for 1 hour, the mixture was diluted with water (20 mL), and extracted with ethyl acetate (20 mL) twice. The combined organic layers were washed with brine (20 mL), dried over Na₂SO₄₍ₛ₎, and filtered. The filtrate was concentrated, and purified by C18 column (acetonitrile:water=20% to 40%) to give the title compound (450 mg, 97% purity, 70% yield) as white solids. LC-MS (ESI): R_{T}=1.59 min, mass calcd. for C₂₅H₂₃Cl₂F₃N₆O₃ 582.1, m/z found 583.4 [M+H]⁺.

Intermediate 4-8

(3R,7S)-2-(3,4-Dichlorobenzoyl)-3-methyl-10-oxo-9-(1-(2-(trifluoromethyl)pyrimidin-5-yl)ethyl)-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-7-(hydroxymethyl)-3-methyl-9-(1-(2-(trifluoromethyl)pyrimidin-5-yl)ethyl)-1,2,3,4,8,9-hexahydropyrido[4′,3′:3,4]pyrazolo[1,5-a]pyrazin-10(7H)-one 4-7 (390 mg, 97% purity, 0.648 mmol) in acetonitrile (5 mL) was added saturated potassium dihydrogen phosphate (5 mL), 2,2,6,6-tetramethylpiperidinooxy (203 mg, 1.3 mmol), sodium chlorite (147 mg, 1.3 mmol), dropwise 5.5% sodium hypochlorite solution (1.5 mL, 1.39 mmol) at 0° C. The reaction mixture was allowed to slowly return to room temperature. After being stirred at room temperature overnight, the reaction mixture was quenched with saturated sodium thiosulfate aqueous solution (10 mL), acidized with 1 M hydrochloric acid solution to pH=4˜5, and extracted with ethyl acetate (10 mL) for three times. The combined organic layers were washed with brine (10 mL), dried over Na₂SO₄₍ₛ₎, and filtered. The filtrate was concentrated and purified by C18 column (acetonitrile:water=5% to 35%) to give the title compound (311 mg, 100% purity, 80% yield) as white solids. LC-MS (ESI): R_{T}=1.28 min, mass calcd. for C₂₅H₂₁Cl₂F₃N₆O₄ 596.1, m/z found 595.1 [M−H]⁻.

Compound 4

(3R,7S)-2-(3,4-Dichlorobenzoyl)-N,3-dimethyl-10-oxo-9-(1-(2-(trifluoromethyl)pyrimidin-5-yl)ethyl)-1,2,3,4,7,8,9,10-octahydropyrido[4′,3′:3,4]pyrazolo[1,5-a]pyrazine-7-carboxamide

To the solution of (3R,7S)-2-(3,4-dichlorobenzoyl)-3-methyl-10-oxo-9-(1-(2-(trifluoromethyl)pyrimidin-5-yl)ethyl)-1,2,3,4,7,8,9,10-octahydropyrido[4′,3′:3,4]pyrazolo[1,5-a]pyrazine-7-carboxylic acid 4-8 (270 mg, 100% purity, 0.452 mmol) in N,N-dimethylformamide (9 mL) was added methanamine hydrochloride (76 mg, 1.13 mmol), 1-hydroxybenzotriazole (122 mg, 0.903 mmol), 1-ethyl-(3-(3-dimethylamino)propyl)-carbodiimide hydrochloride (173 mg, 0.902 mmol) and triethylamine (0.4 mL, 2.88 mmol) at 0° C. The mixture was stirred at room temperature for 3 hours. The mixture was diluted with water (10 mL), acidized with 0.5 M hydrochloric acid aqueous solution to pH˜5 and extracted with ethyl acetate (10 mL) twice. The combined organic layers were washed with brine (10 mL), dried over Na₂SO₄₍ₛ₎ and filtered. The filtrate was concentrated and purified by C18 column (acetonitrile:water=30% to 60%) to give the title compound P1 (210 mg, 100% purity from LCMS, 76% yield) as yellow solids. LC-MS (ESI): R_{T}=1.58 min, mass calcd. for C₂₆H₂₄Cl₂F₃N₇O₃ 609.1, m/z found 610.3 [M+H]⁺.

Compounds 4A and 4B

(3R,7S)-2-(3,4-Dichlorobenzoyl)-N,3-dimethyl-10-oxo-9-((R)-1-(2-(trifluoromethyl)pyrimidin-5-yl)ethyl)-1,2,3,4,7,8,9,10-octahydropyrido[4′,3′:3,4]pyrazolo[1,5-a]pyrazine-7-carboxamide (4A), and (3R,7S)-2-(3,4-dichlorobenzoyl)-N,3-dimethyl-10-oxo-9-((S)-1-(2-(trifluoromethyl)pyrimidin-5-yl)ethyl)-1,2,3,4,7,8,9,10-octahydropyrido[4′,3′:3,4]pyrazolo[1,5-a]pyrazine-7-carboxamide (4B)

A racemic of (3R,7S)-2-(3,4-dichlorobenzoyl)-N,3-dimethyl-10-oxo-9-(1-(2-(trifluoromethyl)pyrimidin-5-yl)ethyl)-1,2,3,4,7,8,9,10-octahydropyrido[4′,3′:3,4]pyrazolo[1,5-a]pyrazine-7-carboxamide compound 4 (280 mg, 100% purity, 0.104 mmol) was separated by chiral Prep. HPLC separation condition: (Column: Chiralpak IC 5 μm 20*250 mm; Mobile Phase: MeOH:EtOH=50:50 at 30 mL/min; Temp: 30° C.; Wavelength: 254 nm) to give compound 4A (33.8 mg, 98.4% purity, 17.6% yield, 99.9% stereopure) as white solids and compound 4B (51.3 mg, 99.3% purity, 27% yield, 100% stereopure) as white solids.

Compound 4A

LC-MS (ESI): R_{T}=3.233 min, mass calcd. for C₂₆H₂₄Cl₂F₃N₇O₃ 609.1, m/z found 610.2 [M+H]⁺. Chiral analysis (Column: Chiralpak IE 5 μm 4.6*250 mm; Mobile Phase: MeOH:EtOH=50:50 at 1 mL/min; Temp: 30° C.; Wavelength: 254 nm; R_{T}=5.195 min). ¹H NMR (400 MHz, CDCl₃) δ 8.83 (s, 2H), 7.53-7.51 (m, 2H), 7.27-7.4 (m, 1H), 5.99-5.44 (m, 3H), 4.89-4.46 (m, 3H), 4.13-4.09 (m, 1H), 3.94-3.90 (m, 1H), 3.05 (br s, 1H), 2.74-2.68 (m, 4H), 1.72-1.70 (m, 3H), 1.32-1.30 (m, 3H). ¹⁹F NMR (376 MHz, CDCl₃) −70.22.

Compound 4B

LC-MS (ESI): R_{T}=2.978 min, mass calcd. for C₂₆H₂₄Cl₂F₃N₇O₃ 609.1, m/z found 610.2 [M+H]⁺. Chiral analysis (Column: Chiralpak ILE 5 μm 4.6*250 mm; Mobile Phase: MeOH:EtOH=50:50 at 1 mL/min; Temp: 30° C.; Wavelength: 254 nm; R_{T}=6.976 min). ¹H NMR (400 MHz, CDCl₃) δ 8.92 (s, 2H), 7.54-7.52 (m, 2H), 7.26-7.24 (m, 1H), 6.09 (br s, 1H), 5.85-5.53 (m, 2H), 4.93-4.30 (m, 4H), 3.64-3.59 (m, 1H), 3.07 (br s, 1H), 2.83-2.70 (m, 4H), 1.76-1.74 (m, 3H), 1.29-1.28 (m, 3H). ¹⁹F NMR (376 MHz, CDCl₃) −70.26.

Compound 4B (Preparation Method B)

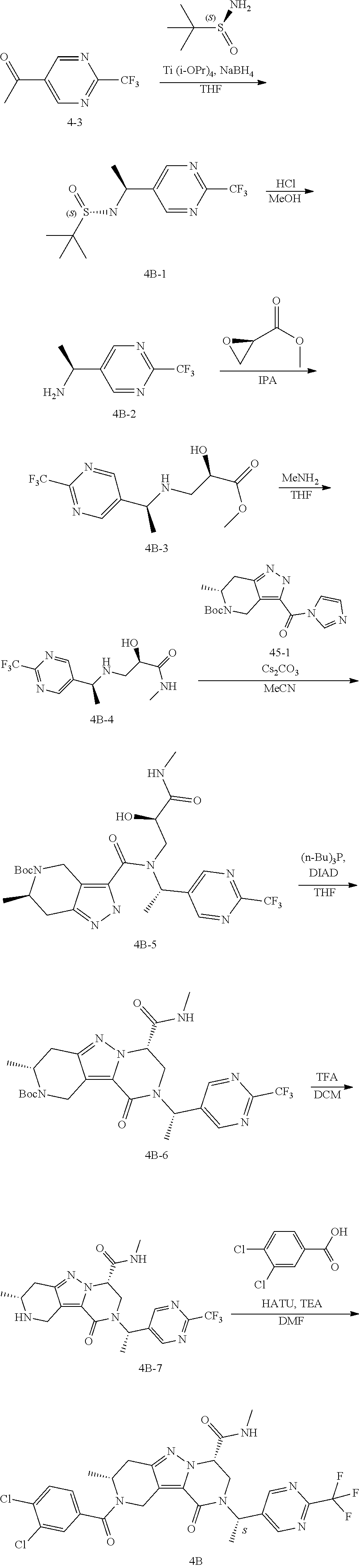

Intermediate 4B-1

(S)-2-Methyl-N—((S)-1-(2-(trifluoromethyl)pyrimidin-5-yl)ethyl)propane-2-sulfinamide

To a solution of 1-(2-(trifluoromethyl)pyrimidin-5-yl)ethanone 4-3 (19 g, 100% purity, 99.9 mmol) and (S)-2-methylpropane-2-sulfinamide (20 g, 165 mmol) in tetrahydrofuran 450 mL) was added titanium(IV) tetraisopropanolate (60 mL, 205 mmol). After stirred at 70° C. for 16 hours, the reaction mixture was cooled to 0° C., then sodium borohydride (2 g, 52.9 mmol) was added and stirred at room temperature for 1 hour. The reaction was quenched with methanol (30 mL), poured into water (60 mL), filtered with kieselguhr. The cake was washed with ethyl acetate (1 L). The filtrate was concentrated, purified by silica gel column chromatography (petroleum ether:ethyl acetate=3:1) to give the title compound (19 g, 96% purity, 62% yield, 100% stereopure) as yellow oil. LC-MS (ESI): R_{T}=1.45 min, mass calcd. for C¹¹H₁₆F₃N₃OS 295.3, m/z found 296.1 [M+H]⁺. Chiral analysis (Column: Chiralpak IG 5 μm 4.6*250 mm; Mobile Phase: Hex:EtOH=80:20 at 1 mL/min; Temp: 30° C.; Wavelength: 254 nm; R_{T}=10.051 min).

Intermediate 4B-2

(S)-1-(2-(trifluoromethyl)pyrimidin-5-yl)ethan-1-amine

To the solution of (S)-2-methyl-N—((S)-1-(2-(trifluoromethyl)pyrimidin-5-yl)ethyl)propane-2-sulfinamide 4B-1 (14 g, 96% purity, 45.5 mmol) in methanol (100 mL) was added 4 M hydrochloride in 1,4-dioxane (50 mL, 200 mmol). The reaction mixture was stirred at 0° C. for 2 hours. The mixture was concentrated and adjusted pH˜8 with saturated sodium bicarbonate aqueous solution, then extracted with ethyl acetate (60 mL) twice. The combined organic layers were washed with brine (120 mL) and dried over Na₂SO₄₍ₛ₎ and filtered. The filtrate was concentrated to give the title compound (7.0 g, 100% purity, 80% yield, 99.7% stereopure) as yellow oil. LC-MS (ESI): R_{T}=1.18 min, mass calcd. for C₇H₈F₃N₃ 191.2, m/z found 192.0 [M+H]⁺. Chiral analysis (Column: Chiralpak ID 5 μm 4.6*250 mm; Mobile Phase: Hex:EtOH:DEA=95:5:0.2 at 1 mL/min; Temp: 30° C.; Wavelength: 254 nm; R_{T}=10.854 min).

Intermediate 4B-3

Methyl-(R)-2-hydroxy-3-(((S)-1-(2-(trifluoromethyl)pyrimidin-5-yl)ethyl)amino)propanoate

(S)-1-(2-(trifluoromethyl)pyrimidin-5-yl)ethan-1-amine 4B-2 (7.0 g, 100% purity, 26.5 mmol), (R)-methyl oxirane-2-carboxylate (5 g, 49.0 mmol) were mixed in propan-2-ol (60 mL) at 30° C. in a sealed tube. After stirred at 60° C. under nitrogen atmosphere for 16 hours, the mixture was concentrated and purified by silica gel column chromatography (petroleum ether:ethyl acetate=1:1) to give the title compound (6.5 g, 79% yield, 95% purity, 99.7% stereopure) as yellow oil. LC-MS (ESI): R_{T}=1.31 min, mass calcd. for C₁₁H₁₄F₃N₃O₃ 293.2, m/z found 294.1 [M+H]⁺. Chiral analysis (Column: Chiralpak IE 5 μm 4.6*250 mm; Mobile Phase: Hex:IPA=90:10 at 1 mL/min; Temp: 30° C.; Wavelength: 254 nm; R_{T}=12.408 min).

Intermediate 4B-4

(R)-2-Hydroxy-N-methyl-3-(((S)-1-(2-(trifluoromethyl)pyrimidin-5-yl)ethyl)amino) propanamide

A solution of methyl-(R)-2-hydroxy-3-(((S)-1-(2-(trifluoromethyl)pyrimidin-5-yl)ethyl)amino)propanoate 4B-3 (6.5 g, 95% purity, 21.1 mmol) in 2 M methylamine in tetrahydrofuran (50 mL, 100 mmol) was stirred at 80° C. for 16 hours in a sealed tube (100 mL). The mixture was concentrated to give the residue and purified by C18 column (acetonitrile:water (+0.02% ammonium acetate)=5% to 100%) to give the title compound (6.2 g, 98% purity, 99% yield) as while solids. LC-MS (ESI): R_{T}=1.16 min, mass calcd. for C¹¹H₁₄F₃N₃O₃ 292.2, m/z found 293.3 [M+H]⁺. Chiral analysis (Column: Chiralpak IH 5 μm 4.6*250 mm; Mobile Phase: Hex:EtOH:DEA=90:10:0.2 at 1 mL/min; Temp: 30° C.; Wavelength: 254 nm; R_{T}=7.772 min).

Intermediate 4B-5

tert-Butyl (6R)-3-(((R)-2-hydroxy-3-(methylamino)-3-oxopropyl)((S)1-(2-(trifluoromethyl)pyrimidin-5-yl)ethyl)carbamoyl)-6-methyl-2,4,6,7-tetrahydro-5H-pyrazolo[4,3-c]pyridine-5-carboxylate

To a solution of tert-butyl (R)-3-(1H-imidazole-1-carbonyl)-6-methyl-2,4,6,7-tetrahydro-5H-pyrazolo[4,3-c]pyridine-5-carboxylate 45-1 (7.5 g, 100% purity, 22.6 mmol), (R)-2-hydroxy-N-methyl-3-(((S)-1-(2-(trifluoromethyl)pyrimidin-5-yl)ethyl) amino)propanamide 4B-4 (6.2 g, 98% purity, 20.8 mmol) in acetonitrile (120 mL) was added cesium carbonate (13 g, 39.9 mmol) at 30° C. After stirred at 45° C. for 3 hours, the mixture was added water (200 mL), then washed with ethyl acetate (200 mL) and brine (200 mL), dried over Na₂SO₄₍ₛ₎ and filtered. The filtrate was concentrated and purified by silica gel column chromatography (dichloromethane:methanol=10:1) to give the title compound (7.6 g, 100% purity, 66% yield) as light yellow oil. LC-MS (ESI): R_{T}=2.29 min, mass calcd. for C₂₄H₃₂F₃N₇O₅ 555.6, m/z found 556.2 [M+H]⁺. Chiral analysis (Column: Chiralpak IC 5 m 4.6*250 mm; Mobile Phase: Hex:EtOH:DEA=85:15:0.2 at 1 mL/min; Temp: 30° C.; Wavelength: 254 nm; R_{T}=14.702 min).

Intermediate 4B-6

tert-Butyl (3R,7S)-3-methyl-7-(methylcarbamoyl)-10-oxo-9-((S)-1-(2-(trifluoromethyl) pyrimidin-5-yl)ethyl)-3,4,7,8,9,10-hexahydropyrido[4′,3′:3,4] pyrazolo[1,5-a]pyrazine-2(1H)-carboxylate

Tert-butyl (6R)-3-(((R)-2-hydroxy-3-(methylamino)-3-oxopropyl)((S)-1-(2-(trifluoromethyl)pyrimidin-5-yl)ethyl)carbamoyl)-6-methyl-2,4,6,7-tetrahydro-5H-pyrazolo[4,3-c]pyridine-5-carboxylate 4B-5 (7.6 g, 100% purity, 13.7 mmol), tributylphosphane (4.2 g, 20.8 mmol) and (E)-diisopropyl diazene-1,2-dicarboxylate (4.2 g, 20.8 mmol) were mixed in tetrahydrofuran (120 mL) at 0° C. After stirred at 0° C. for 2 hours, the reaction mixture was concentrated under reduced pressure to give a residue, which was purified by C18 column (acetonitrile:water (+0.02% ammonium acetate)=5% to 100%) to give the title compound (13 g, 50% purity (including 50% Bu₃PO MS: [219]), 88% yield) as yellow oil. LC-MS (ESI): R_{T}=2.64 min and 2.67 min, mass calcd. for C₂₄H₃₀F₃N₇O₄ 537.5, m/z found 555.2 [M+18]⁺.

Intermediate 4B-7

(3R,7S)—N,3-Dimethyl-10-oxo-9-((S)-1-(2-(trifluoromethyl)pyrimidin-5-yl)ethyl)-1,2,3,4,7,8,9,10-octahydropyrido[4′,3′:3,4]pyrazolo[1,5-a]pyrazine-7-carboxamide

To the solution of tert-butyl (3R,7S)-3-methyl-7-(methylcarbamoyl)-10-oxo-9-((S)-1-(2-(trifluoromethyl)pyrimidin-5-yl)ethyl)-3,4,7,8,9,10-hexahydropyrido[4′,3′:3,4]pyrazolo [1,5-a]pyrazine-2(1H)-carboxylate 4B-6 ((9 g, 50% purity, 8.37 mmol) in dichloromethane (60 mL) was added trifluoroacetic acid (15 mL) at 0° C. After stirred at 0° C. for 2 hours, the mixture was concentrated and adjusted to pH˜8 with saturated sodium bicarbonate aqueous solution, then extracted with dichloromethane (100 mL) twice. The combined organic layers were washed with brine (150 mL) and dried over Na₂SO₄₍ₛ₎ and filtered. The filtrate was concentrated to give the title compound (3 g, 75% yield, 91% purity) as yellow oil. LC-MS (ESI): R_{T}=1.76 min and 1.88 min, mass calcd. for C₁₉H₂₂F₃N₇O₂ 437.1, m/z found 436.5 [M−H]⁻.

Compound 4B

(3R,7S)-2-(3,4-Dichlorobenzoyl)-N,3-dimethyl-10-oxo-9-((S)-1-(2-(trifluoromethyl) pyrimidin-5-yl)ethyl)-1,2,3,4,7,8,9,10-octahydropyrido[4′,3′:3,4]pyrazolo[1,5-a]pyrazine-7-carboxamide

(3R,7S)—N,3-Dimethyl-10-oxo-9-((S)-1-(2-(trifluoromethyl)pyrimidin-5-yl)ethyl)-1,2,3,4,7,8,9,10-octahydropyrido[4′,3′:3,4]pyrazolo[1,5-a]pyrazine-7-carboxamide 4B-7 (3.0 g, 91% purity, 6.2 mmol), 3,4-dichlorobenzoic acid (1.5 g, 7.85 mmol), N-ethyl-N-isopropylpropan-2-amine (3 mL, 16.2 mmol) and O-(7-azabenzotriazol-1-yl)-N,N,N′,N′-tetramethyluronium hexafluorophosphate (3 g, 7.89 mmol) were mixed in N,N-dimethylformamide (45 mL) at 0° C. After stirred at 30° C. for 1 hour, the mixture was concentrated and purified by C18 column (acetonitrile:water (+0.02% ammonium acetate)=5% to 100%) to give the title crude compound (3.2 g, 100% purity, 84% yield) as white solids. LC-MS (ESI): R_{T}=3.26 min, mass calcd. for C₂₆H₂₄Cl₂F₃N₇O₃ 609.1, m/z found 608.6 [M−H]⁻. Then 4B ((3.2 g, 100% purity, 5.24 mmol) was separated by chiral HPLC (separation condition: Column: Chiralpak IC 5 μm 30*250 mm; Mobile Phase: ACN=100 at 20 mL/min; Temp: 30° C.; Wavelength: 214 nm) to afford the title product (2.8 g, 99.8% purity, recovery yield 87.3%, 100% stereopure) as white solids. LC-MS (ESI): R_{T}=8.337 min, mass calcd. for C₂₆H₂₄Cl₂F₃N₇O₃ 609.1, m/z found 610.0 [M+H]⁺. Chiral analysis (Column: Chiralpak IC 5 μm 4.6*250 mm; Mobile Phase: ACN=100% at 1 mL/min; Temp: 30° C.; Wavelength: 214 nm; R_{T}=10.028 min). ¹H NMR (400 MHz, CDCl₃) δ 8.92 (s, 2H), 7.54-7.51 (m, 2H), 7.26-7.24 (m, 1H), 6.08-5.44 (m, 3H), 4.93-4.30 (m, 4H), 3.65-3.60 (m, 1H), 3.14-2.96 (m, 1H), 2.83-2.70 (m, 4H), 1.75 (d, J=7.2 Hz, 3H), 1.28 (d, J=6.8 Hz, 3H). ¹⁹F NMR (376 MHz, CDCl₃) δ −70.28.

Compounds 5A and 5B

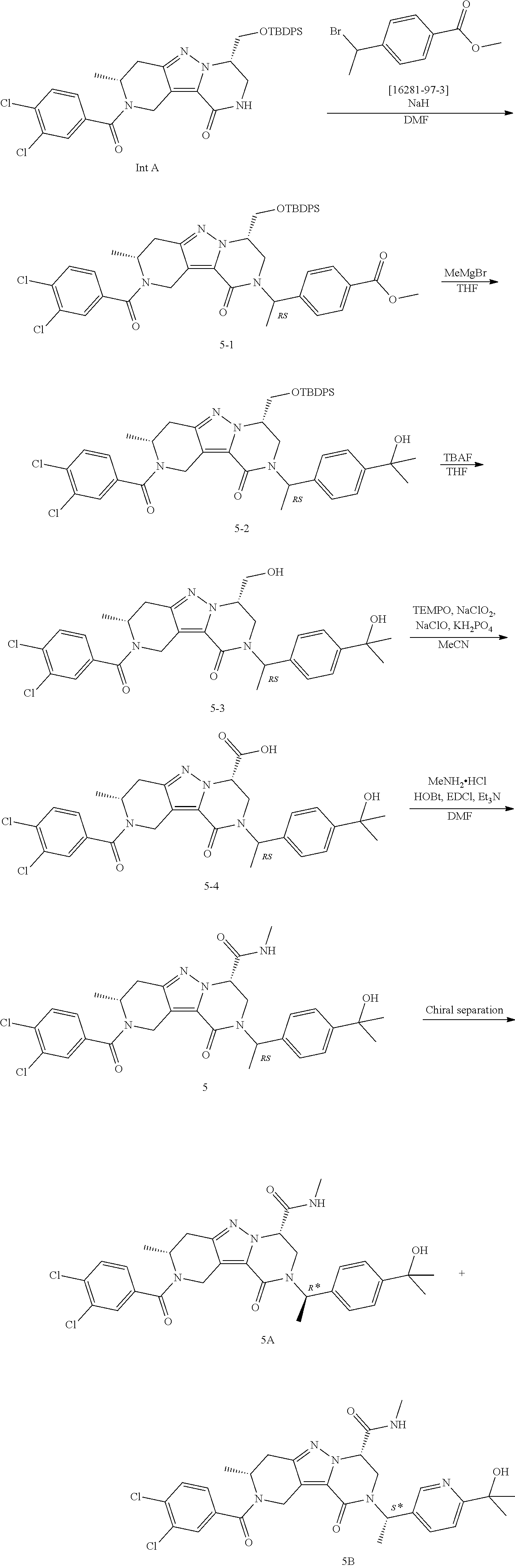

Intermediate 5-1

Methyl 4-(1-((3R,7S)-7-(((tert-butyldiphenylsilyl)oxy)methyl)-2-(3,4-dichlorobenzoyl)-3-methyl-10-oxo-1,2,3,4,7,8-hexahydropyrido[4′,3′:3,4]pyrazolo[1,5-a]pyrazin-9(10H)-yl)ethyl)benzoate

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (1.40 g, 2.03 mmol) in dimethylformamide (15 mL) was added sodium hydride, 60% dispersion in the mineral oil (81 mg, 2.03 mmol). The mixture was stirred at 0° C. for 1 hour. Then methyl 4-(1-bromoethyl)benzoate (490 mg, 2.02 mmol) was added to the mixture. The reaction mixture was stirred at 0° C. for 1 hour. The reaction mixture was quenched by saturated ammonium chloride (30 mL) and extracted with ethyl acetate (50 mL×3). The combined organic layers were washed by water (50 mL×2) and brine (50 mL), dried over Na₂SO₄₍ₛ₎, filtered and concentrated under reduced pressure to give a residue, which was purified by silica gel column (petroleum ether:ethyl acetate=1:1) to give the title compound (900 mg, 95% purity from LCMS, yield 52%) as a yellow solid. LC-MS (ESI): R_{T}=1.77 min & 1.81 min, mass calcd. for C₄₄H₄₆Cl₂N₄O₅Si 808.3 m/z found 808.9 [M+H]+; ¹H NMR (400 MHz, CDCl₃) δ 8.04-7.92 (m, 2H), 7.65-7.32 (m, 14H), 7.24-7.21 (m, 1H), 6.01-5.40 (m, 2H), 4.82-4.29 (m, 3H), 4.08-4.03 (m, 1H), 3.92-3.89 (m, 3H), 3.86-3.30 (m, 3H), 3.10-2.91 (m, 1H), 2.64-2.53 (m, 11H), 1.56-1.55 (m, 3H), 1.21 (br s, 3H), 1.02-0.90 (m, 9H).

Intermediate 5-2

(3R,7S)-7-(((tert-Butyldiphenylsilyl)oxy)methyl)-2-(3,4-dichlorobenzoyl)-9-(1-(4-(2-hydroxypropan-2-yl)phenyl)ethyl)-3-methyl-1,2,3,4,8,9-hexahydropyrido[4′,3′:3,4]pyrazolo[1,5-a]pyrazin-10(7H)-one

The solution of methyl 4-(1-((3R,7S)-7-(((tert-butyldiphenylsilyl)oxy)methyl)-2-(3,4-dichlorobenzoyl)-3-methyl-10-oxo-1,2,3,4,7,8-hexahydropyrido[4′,3′:3,4]pyrazolo[1,5-a]pyrazin-9(10H)-yl)ethyl)benzoate 5-1 (750 mg, 0.87 mmol) in tetrahydrofuran (15 mL) was cooled to 0° C. Then methylmagnesium bromide, 1.0 M solution in tetrahydrofuran (4 mL, 4.00 mmol) was added dropwise within 20 min. The reaction mixture was stirred at 0° C. for 2 hours. The reaction mixture was quenched by saturated aqueous ammonium chloride solution (50 mL) and extracted with ethyl acetate (50 mL×3). The combined organic layers were washed by brine (50 mL), dried over Na₂SO₄₍ₛ₎, filtered and concentrated under reduced pressure to give the title compound (540 mg, 92% purity from LCMS, yield 70%) as a yellow solid. LC-MS (ESI): R_{T}=1.76 min & 1.79 min, mass calcd. for C₄₅H₅₀Cl₂N₄O₄Si 808.3 m/z found 809.0 [M+H]⁺.

Intermediate 5-3

(3R,7S)-2-(3,4-Dichlorobenzoyl)-7-(hydroxymethyl)-9-(1-(4-(2-hydroxypropan-2-yl)phenyl)ethyl)-3-methyl-1,2,3,4,8,9-hexahydropyrido[4′,3′:3,4]pyrazolo[1,5-a]pyrazin-10(7H)-one

The solution of (3R,7S)-7-(((tert-butyldiphenylsilyl)oxy)methyl)-2-(3,4-dichlorobenzoyl)-9-(1-(4-(2-hydroxypropan-2-yl)phenyl)ethyl)-3-methyl-1,2,3,4,8,9-hexahydropyrido[4′,3′:3,4]pyrazolo[1,5-a]pyrazin-10(7H)-one 5-2 (540 mg, 0.610 mmol) in tetrahydrofuran (10 mL) was cooled to 0° C. Then tetrabutylammonium fluoride, 1.0 M solution in THE (1.0 mL, 1.00 mmol) was added dropwise. The reaction mixture was stirred at 0° C. for 1 hour. The reaction mixture was concentrated under reduced pressure and purified by silica gel column (petroleum ether:ethyl acetate=1:2) to give the title compound (340 mg, 91% purity from LCMS, yield 88%) as a yellow solid. LC-MS (ESI): R_{T}=1.32 min & 1.35 min, mass calcd. for C₂₉H₃₂Cl₂N₄O₄ 570.2 m/z found 553.3 [M+H−18]⁺.

Intermediate 5-4

(3R,7S)-2-(3,4-Dichlorobenzoyl)-9-(1-(4-(2-hydroxypropan-2-yl)phenyl)ethyl)-3-methyl-10-oxo-1,2,3,4,7,8,9,10-octahydropyrido[4′,3′:3,4]pyrazolo[1,5-a]pyrazine-7-carboxylic acid

To a suspension of (3R,7S)-2-(3,4-dichlorobenzoyl)-7-(hydroxymethyl)-9-(1-(4-(2-hydroxypropan-2-yl)phenyl)ethyl)-3-methyl-1,2,3,4,8,9-hexahydropyrido[4′,3′:3,4]pyrazolo[1,5-a]pyrazin-10(7H)-one 5-3 (340 mg, 0.54 mmol) in acetonitrile (4 mL) was added saturated potassium dihydrogen phosphate solution in water (4 mL). The mixture was cooled to 0° C. Then 2,2,6,6-tetramethylpiperidinyloxy (170 mg, 1.09 mmol), sodium chlorite (123 mg, 1.09 mmol) and sodium hypochlorite (0.65 mL, 1.09 mmol) were added. The reaction mixture was stirred at room temperature for 16 hours. The reaction mixture was adjusted pH to 5-6 by 1 M hydrochloride solution in water and extracted with dichloromethane (50 mL) three times. The combined organic layers were washed by brine (50 mL), dried over Na₂SO₄₍ₛ₎, filtered and concentrated under reduced pressure to give a residue, which was purified by silica gel column (dichloromethane:methanol=5:1) to give the title compound (250 mg, 93% purity from LCMS, yield 73%) as a yellow solid. LC-MS (ESI): R_{T}=0.68 min, mass calcd. for C₂₉H₃₀Cl₂N₄O₅ 584.2 m/z found 567.0 [M+H−18]⁺.

Compound 5

(3R,7S)-2-(3,4-Dichlorobenzoyl)-9-(1-(4-(2-hydroxypropan-2-yl)phenyl)ethyl)-N,3-dimethyl-10-oxo-1,2,3,4,7,8,9,10-octahydropyrido[4′,3′:3,4]pyrazolo[1,5-a]pyrazine-7-carboxamide

To a mixture of (3R,7S)-2-(3,4-dichlorobenzoyl)-9-(1-(4-(2-hydroxypropan-2-yl)phenyl)ethyl)-3-methyl-10-oxo-1,2,3,4,7,8,9,10-octahydropyrido[4′,3′:3,4]pyrazolo[1,5-a]pyrazine-7-carboxylic acid 5-4 (250 mg, 0.40 mmol) and methylamine hydrochloride (54 mg, 0.80 mmol) in dimethylformamide (4 mL) was added 1-hydroxybenzotrizole (108 mg, 0.80 mmol) and 1-(3-dimethylaminopropyl)-3-ethylcarbodiimide hydrochloride (153 mg, 0.80 mmol). The mixture was cooled to 0° C. and triethylamine (121 mg, 1.20 mmol) solution in dimethylformamide (1 mL) was added dropwise within 30 min. The reaction mixture was stirred at 0° C. for 1 hour. The reaction mixture was quenched by saturated ammonium chloride aqueous solution (40 mL) and extracted with ethyl acetate (40 mL×3). The combined organic layers were washed by water (30 mL×3) and brine (30 mL), dried over Na₂SO₄₍ₛ₎, filtered and concentrated under reduced pressure to give a residue, which was purified by silica gel column (dichloromethane:methanol=12:1) to give the title compound (190 mg, 97% purity from LCMS, yield 78%) as a yellow solid. LC-MS (ESI): R_{T}=0.68 min, mass calcd. for C₃₀H₃₃Cl₂N₅O₄ 597.2 m/z found 580.3 [M+H−18]⁺

Compounds 5A and 5B

(3R,7S)-2-(3,4-Dichlorobenzoyl)-9-((R*)-1-(4-(2-hydroxypropan-2-yl)phenyl)ethyl)-N,3-dimethyl-10-oxo-1,2,3,4,7,8,9,10-octahydropyrido[4′,3′:3,4]pyrazolo[1,5-a]pyrazine-7-carboxamide (5A), and (3R,7S)-2-(3,4-Dichlorobenzoyl)-9-((S*)-1-(4-(2-hydroxypropan-2-yl)phenyl)ethyl)-N,3-dimethyl-10-oxo-1,2,3,4,7,8,9,10-octahydropyrido[4′,3′:3,4]pyrazolo[1,5-a]pyrazine-7-carboxamide (5B)

A racemic of (3R,7S)-2-(3,4-dichlorobenzoyl)-9-(1-(4-(2-hydroxypropan-2-yl)phenyl)ethyl)-N,3-dimethyl-10-oxo-1,2,3,4,7,8,9,10-octahydropyrido[4′,3′:3,4]pyrazolo[1,5-a]pyrazine-7-carboxamide (280 mg, 0.47 mmol) compound 5 (280 mg, 0.47 mmol) was separated by chiral Prep. HPLC separation condition: (Column: Chiralpak IE 250 mm*4.6 mm 5 μm; Mobile Phase: Hex:EtOH=30:70 at 30 mL/min; Temp: 30° C.; Wavelength: 254 nm) to give compound 5A (93.9 mg, 99.0% purity, 34.2% yield, 100% stereopure) as white solids and compound 5B (68.5 mg, 97.1% purity, 24.5% yield, 100% stereopure) as white solids.

Compound 5A

LC-MS (ESI): R_{T}=3.292 min, mass calcd. for C₃₀H₃₃Cl₂N₅O₄ 597.2, m/z found 620.2 [M+Na]⁺. Chiral analysis (Column: Chiralpak IE 5 μm 250 mm*4.6 mm; Mobile Phase: Hex:EtOH=30:70 at 1.0 mL/min; Temp: 30° C.; Wavelength: 254 nm; R_{T}=8.663 min). ¹H NMR (400 MHz, CDCl₃) δ 7.52-7.43 (m, 4H), 7.27-7.24 (m, 4H), 6.12-5.45 (m, 3H), 4.80-4.25 (m, 3H), 3.90-3.73 (m, 2H), 3.17-2.96 (m, 1H), 2.71-2.66 (m, 4H), 1.60 (s, 3H), 1.56 (s, 6H), 1.29-1.28 (m, 3H).

Compound 5B

LC-MS (ESI): R_{T}=4.387 min, mass calcd. for C₃₀H₃₃Cl₂N₅O₄ 597.2, m/z found 620.2 [M+H]⁺. Chiral analysis (Column: Chiralpak IE 5 μm 250 mm*4.6 mm; Mobile Phase: Hex:EtOH=30:70 at 1.0 mL/min; Temp: 30° C.; Wavelength: 254 nm; R_{T}=12.077 min). ¹H NMR (400 MHz, CDCl₃) δ 7.47-7.39 (m, 4H), 7.24-7.19 (m, 4H), 5.92-5.30 (m, 3H), 4.75-4.33 (m, 3H), 4.01-3.98 (m, 1H), 3.35-3.31 (m, 1H), 3.05-2.91 (m, 1H), 2.74-2.72 (m, 3H), 2.66-2.63 (m, 1H), 1.53-1.50 (m, 9H), 1.25-1.23 (m, 3H).

Compounds 6A and 6B (Preparation Method A)

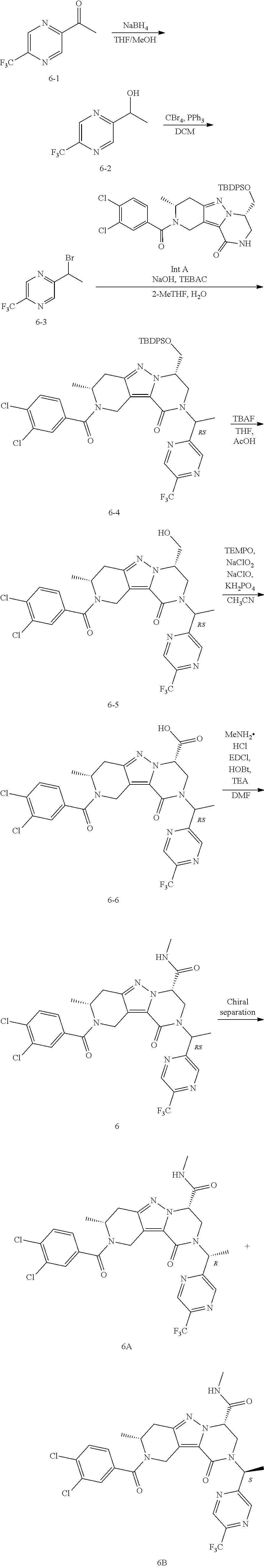

Intermediate 6-2

1-(5-(Trifluoromethyl)pyrazin-2-yl)ethanol

To a solution of 1-(5-(trifluoromethyl)pyrazin-2-yl)ethanone 6-1 (1.2 g, 95% purity, 6.00 mmol) in tetrahydrofuran (24 mL) was added sodium tetrahydroborate (320 mg, 8.46 mmol) and methanol (6 mL). After being stirred at 0° C. for 1 hour, the mixture was quenched with water, and extracted with ethyl acetate (50 mL) for three times. The organic layers were combined, washed with brine (50 mL), dried over Na₂SO₄₍ₛ₎ and filtered. The filtrate was concentrated to get the title compound (1.1 g, 78% purity from LCMS, 74% yield) as a yellow oil. LC-MS (ESI): R_{T}=1.063 min, mass calcd. for C₇H₇F₃N₂O 192.05, m/z found 193.2 [M+H]⁺.

Intermediate 6-3

2-(1-Bromoethyl)-5-(trifluoromethyl)pyrazine

To a solution of 1-(5-(trifluoromethyl)pyrazin-2-yl)ethanol 6-2 (1.1 g, 78% purity, 4.47 mmol) in dichloromethane (22 mL) at 0° C. under nitrogen atmosphere was added carbon tetrabromide (2.5 g, 7.54 mmol) and triphenylphosphine (2.0 g, 7.63 mmol). The reaction mixture was stirred at 0° C. for 1 hour. TLC showed 6-2 was consumed. The reaction mixture was purified by silica gel column chromatography (petroleum ether:ethyl acetate=10:1 to 5:1) to get the title compound (920 mg, 90% purity from ¹H NMR, 73% yield) as a yellow oil. ¹H NMR (400 MHz, CDCl₃) δ 8.89 (s, 1H), 8.85 (s, 1H), 5.29 (q, J=6.8 Hz, 1H), 2.13 (d, J=7.2 Hz, 3H).

Intermediate 6-4

(3R,7S)-7-(((tert-Butyldiphenylsilyl)oxy)methyl)-2-(3,4-dichlorobenzoyl)-3-methyl-9-(1-(5-(trifluoromethyl)pyrazin-2-yl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (700 mg, 90% purity, 0.97 mmol) in 2-methyltetrahydrofuran (6 mL) and 50% wt. sodium hydroxide in water (6 mL) was added 2-(1-bromoethyl)-5-(trifluoromethyl)pyrazine 6-3 (350 mg, 90% purity, 0.35 mmol) and N-benzyl-N,N-diethylethanaminium chloride (35 mg, 0.15 mmol) at 0° C. The mixture was stirred at room temperature for 2 hours. The reaction mixture was quenched with water (50 mL) and extracted with ethyl acetate (50 mL) twice. The combined organic layers were washed with brine (50 mL), dried over Na₂SO₄₍ₛ₎ and filtered. The filtrate was concentrated to give the title compound (1.0 g, 67% purity from LCMS, 84% yield) as a yellow oil. LC-MS (ESI): R_{T}=2.07 min, mass calcd. for C₄₁H₄₁Cl₂F₃N₆O₃Si 820.23, m/z found 821.2 [M+H]⁺.

Intermediate 6-5

(3R,7S)-2-(3,4-Dichlorobenzoyl)-7-(hydroxymethyl)-3-methyl-9-(1-(5-(trifluoromethyl)pyrazin-2-yl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-9-(1-(5-(trifluoromethyl)pyrazin-2-yl)ethyl)-1,2,3,4,8,9-hexahydropyrido[4′,3′:3,4]pyrazolo[1,5-a]pyrazin-10(7H)-one 6-4 (1.5 g, 67% purity, 1.06 mmol) in tetrahydrofuran (10 mL) was added 1 M tetrabutylammonium fluoride in tetrahydrofuran (1.4 mL, 1.4 mmol) and acetic acid (130 mg, 2.17 mmol). The reaction mixture was stirred at room temperature for 2 hours. The saturated aqueous ammonium chloride solution (10 mL) was added to the reaction mixture. The reaction mixture was extracted with ethyl acetate (50 mL) for three times. The combined organic layers were washed with brine (50 mL), dried over Na₂SO₄₍ₛ₎, and filtered. The filtrate was concentrated and purified by C18 column (acetonitrile:water=5% to 90%) to give the title compound (430 mg, 100% purity from LCMS, 70% yield) as a yellow solid. LC-MS (ESI): R_{T}=1.61 min, mass calcd. for C₂₅H₂₃Cl₂F₃N₆O₃ 582.12, m/z found 583.2 [M+H]⁺.

Intermediate 6-6

(3R,7S)-2-(3,4-Dichlorobenzoyl)-3-methyl-10-oxo-9-(1-(5-(trifluoromethyl)pyrazin-2-yl)ethyl)-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-7-(hydroxymethyl)-3-methyl-9-(1-(5-(trifluoromethyl)pyrazin-2-yl)ethyl)-1,2,3,4,8,9-hexahydropyrido[4′,3′:3,4]pyrazolo[1,5-a]pyrazin-10(7H)-one 6-5 (430 mg, 100% purity, 0.74 mmol) in acetonitrile (4 mL) was added saturated potassium dihydrogen phosphate solution (4 mL), 2,2,6,6-tetramethylpiperidinooxy (250 mg, 1.60 mmol), sodium chlorite (190 mg, 1.68 mmol) and dropwise 10% sodium hypochlorite solution (1.0 mL, 1.68 mmol) at 0° C. The reaction mixture was allowed to slowly return to room temperature. After being stirred at room temperature overnight. The reaction mixture was quenched with saturated sodium thiosulfate aqueous solution (20 mL), acidized with 1 M hydrochloric acid solution to pH=4˜5, and extracted with ethyl acetate (50 mL) for three times. The combined organic layers were washed with brine (50 mL), dried over Na₂SO₄₍ₛ₎, and filtered. The filtrate was concentrated and purified by C18 column (acetonitrile:water=5% to 45%) to give the title compound (380 mg, 97% purity from LCMS, 84% yield) as a white solid. LC-MS (ESI): R_{T}=1.33 min, mass calcd. for C₂₅H₂₁Cl₂F₃N₆O₄ 596.10, m/z found 595.2 [M−H]⁻.

Compound 6

(3R,7S)-2-(3,4-Dichlorobenzoyl)-N,3-dimethyl-10-oxo-9-(1-(5-(trifluoromethyl)pyrazin-2-yl)ethyl)-1,2,3,4,7,8,9,10-octahydropyrido[4′,3′:3,4]pyrazolo[1,5-a]pyrazine-7-carboxamide

To a solution of (3R,7S)-2-(3,4-dichlorobenzoyl)-3-methyl-10-oxo-9-(1-(5-(trifluoromethyl)pyrazin-2-yl)ethyl)-1,2,3,4,7,8,9,10-octahydropyrido[4′,3′:3,4]pyrazolo[1,5-a]pyrazine-7-carboxylic acid 6-6 (325 mg, 97% purity, 0.53 mmol), methanamine hydrochloride (92 mg, 1.36 mmol), N1-((ethylimino)methylene)-N3,N3-dimethylpropane-1,3-diamine hydrochloride (210 mg, 1.10 mmol) and 1H-benzo[d][1,2,3]triazol-1-ol (150 mg, 1.11 mmol) in N,N-dimethylformamide (8 mL) was added trimethylamine (0.5 mL, 3.61 mmol) at 0° C. The reaction mixture was allowed to slowly return to room temperature. After being stirred at room temperature under nitrogen atmosphere for 3 hours. The reaction mixture was quenched with water (10 mL) and extracted with ethyl acetate (25 mL) for three times. The combined organic layers were dried over Na₂SO₄₍ₛ₎ and filtered. The filtrate was concentrated and purified by C18 column (acetonitrile:water=5% to 100%) to give the title compound (235 mg, 100% purity from LCMS, 73% yield) as a white solid. LC-MS (ESI): R_{T}=1.60 min, mass calcd. for C₂₆H₂₄Cl₂F₃N₇O₃ 609.13, m/z found 610.2 [M+H]⁺.

Compounds 6A and 6B

(3R,7S)-2-(3,4-Dichlorobenzoyl)-N,3-dimethyl-10-oxo-9-((R)-1-(5-(trifluoromethyl)pyrazin-2-yl)ethyl)-1,2,3,4,7,8,9,10-octahydropyrido[4′,3′:3,4]pyrazolo[1,5-a]pyrazine-7-carboxamide (6A), and (3R,7S)-2-(3,4-Dichlorobenzoyl)-N,3-dimethyl-10-oxo-9-((S)-1-(5-(trifluoromethyl)pyrazin-2-yl)ethyl)-1,2,3,4,7,8,9,10-octahydropyrido[4′,3′:3,4]pyrazolo[1,5-a]pyrazine-7-carboxamide (6B)

A racemic mixture of (3R,7S)-2-(3,4-dichlorobenzoyl)-N,3-dimethyl-10-oxo-9-(1-(5-(trifluoromethyl)pyrazin-2-yl)ethyl)-1,2,3,4,7,8,9,10-octahydropyrido[4′,3′:3,4]pyrazolo[1,5-a]pyrazine-7-carboxamide compound 6 (275 mg, 100% purity, 0.45 mmol) was separated by chiral prep. HPLC (separation condition: Column: Chiralpak IE 5 μm 20*250 mm; Mobile Phase: Hex:EtOH=30:70 at 60 g/min; Temp: 30° C.; Wavelength: 254 nm.) to afford the compound 6A (68.1 mg, 24.6% yield, 99.5% purity, 100% stereopure) as a white solid and compound 6B (89.9 mg, 32.6% yield, 99.7% purity, 99.9% stereopure) as a white solid.

Compound 6A

LC-MS (ESI): R_{T}=3.621 min, mass calcd. for C₂₆H₂₄Cl₂F₃N₇O₃ 609.13, m/z found 610.2 [M+H]⁺. Chiral analysis (Column: Chiralpak IE 5 μm 4.6*250 mm; Mobile Phase: Hex:EtOH=30:70 at 1 mL/min; Col. Temp: 30° C.; Wavelength: 254 nm, R_{T}=7.403 min). ¹H NMR (400 MHz, DMSO-d₆) δ 9.16 (s, 1H), 8.74-8.64 (m, 1H), 7.80-7.74 (m, 3H), 7.46 (d, J=8.0 Hz, 1H), 6.07-5.88 (m, 1H), 5.43-5.23 (m, 1H), 5.03 (s, 1H), 4.62-4.40 (m, 1H), 4.21-4.06 (m, 2H), 3.73-3.59 (m, 1H), 2.97-2.85 (m, 1H), 2.63-2.53 (m, 1H), 2.34 (d, J=4.4 Hz, 3H), 1.60-1.54 (m, 3H), 1.24-1.17 (m, 3H). ¹⁹F NMR (376 MHz, DMSO-d₆) δ −66.20.

Compound 6B

LC-MS (ESI): R_{T}=3.704 min, mass calcd. for C₂₆H₂₄Cl₂F₃N₇O₃ 609.13, m/z found 610.2 [M+H]⁺. Chiral analysis (Column: Chiralpak IE 5 μm 4.6*250 mm; Mobile Phase: Hex:EtOH=30:70 at 1 mL/min; Col. Temp: 30° C.; Wavelength: 254 nm, R_{T}=9.817 min). ¹H NMR (400 MHz, DMSO-d₆) δ 9.15 (s, 1H), 8.97-8.86 (m, 1H), 8.11 (s, 1H), 7.74-7.72 (m, 2H), 7.45-7.42 (m, 1H), 5.86-5.77 (m, 1H), 5.39-5.10 (m, 2H), 4.53-4.11 (m, 2H), 3.93 (s, 2H), 2.98-2.84 (m, 1H), 2.65 (d, J=4.4 Hz, 3H), 2.59-2.55 (m, 1H), 1.62-1.48 (m, 3H), 1.23-1.11 (m, 3H). ¹⁹F NMR (376 MHz, DMSO-d₆) δ −66.13.

Compound 6B (Preparation Method B)

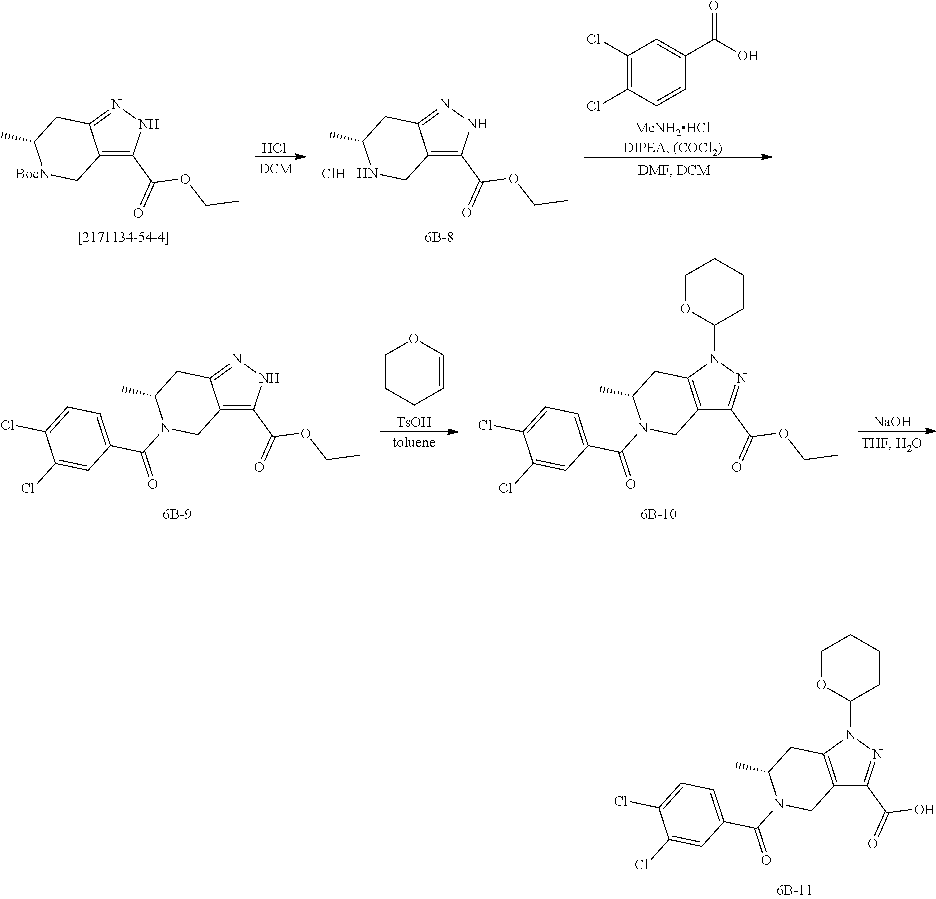

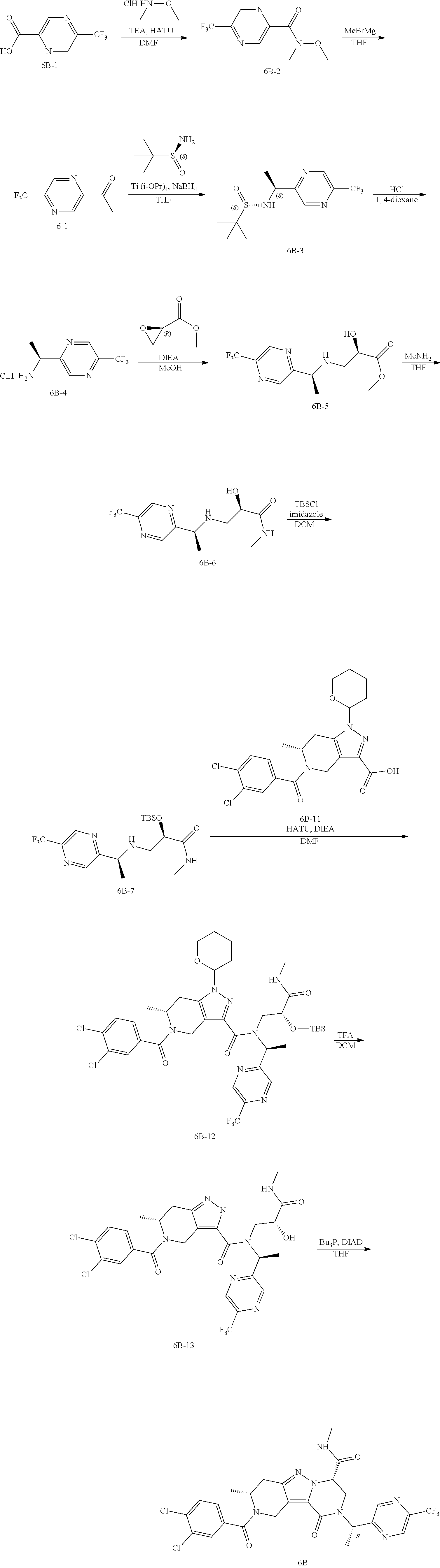

Intermediate 6B-2

N-Methoxy-N-methyl-5-(trifluoromethyl)pyrazine-2-carboxamide

To the solution of 5-(trifluoromethyl)pyrazine-2-carboxylic acid 6B-1 (13 g, 67.7 mmol) and N,O-dimethylhydroxylamine hydrochloride (5.0 g, 81.9 mmol) in N,N-dimethylformamide (70 mL) were added triethylamine (30 mL, 216 mmol) and O-(7-azabenzotriazol-1-yl)-N,N,N′,N′-tetramethyluronium hexafluorophosphate (38 g, 99.9 mmol) at 0° C. After stirred at room temperature overnight, the mixture was diluted with water (250 mL), extracted with ethyl acetate (200 mL) twice. The combined organic layers were washed with brine (100 mL), dried over Na₂SO₄₍ₛ₎, filtered. The filtrate was concentrated, purified by silica gel column chromatography (petroleum ether:acetone=10:1 to 3:1) to give the title compound (14 g, 90% purity from ¹HNMR, 79% yield) as yellow solids. LC-MS (ESI): R_{T}=1.41 min, mass calcd. for C₈H₈F₃N₃O₂ 235.1, m/z found 236.1 [M+H]⁺. ¹H NMR (400 MHz, CDCl₃) δ 8.97 (s, 2H), 3.76 (s, 3H), 3.43 (s, 3H).

Intermediate 6-1

1-(5-(Trifluoromethyl)pyrazin-2-yl)ethanone

To a solution of N-methoxy-N-methyl-5-(trifluoromethyl)pyrazine-2-carboxamide 6B-2 (14.0 g, 90% purity, 53.6 mmol) in tetrahydrofuran (140 mL) at −78° C. was added 1 M methylmagnesium bromide in tetrahydrofuran (70 mL, 70 mmol) under nitrogen atmosphere. The reaction mixture was stirred at −78° C. for 2 hours, then quenched with saturated ammonium chloride aqueous solution (50 mL). The mixture was extracted with dichloromethane (100 mL) for three times. The combined organic phases were washed with brine (100 mL), dried over Na₂SO₄₍ₛ₎, filtered, concentrated to give the crude product which was purified by silica gel column chromatography (petroleum ether:ethyl acetate=20:1 to 5:1) to give the title compound (4.0 g, 95% purity from ¹HNMR, 37% yield) as yellow solids. ¹H NMR (400 MHz, CDCl₃) δ 9.32 (s, 1H), 9.02 (s, 1H), 2.77 (s, 3H).

Intermediate 6B-3

(S)-2-Methyl-N—((S)-1-(5-(trifluoromethyl)pyrazin-2-yl)ethyl)propane-2-sulfinamide

To a solution of 1-(5-(trifluoromethyl)pyrazin-2-yl)ethanone 6-1 (5.0 g, 26.3 mmol) in tetrahydrofuran (50 mL) were added (S)-(−)-2-methyl-2-propanesulfinamide (6.4 g, 52.8 mmol) and titanium(IV) tetraisopropanolate (7.5 g, 26.4 mmol). After stirred at room temperature overnight, the reaction mixture was cooled to 0° C., then sodium tetrahydroborate (1.0 g, 26.4 mmol) was added. After stirred at room temperature for 1 hour, the mixture was diluted with water (100 mL) and extracted with ethyl acetate (100 mL) for three times. The combined organic layers were washed with brine (150 mL), dried over Na₂SO₄₍ₛ₎ and filtered. The filtrate was concentrated to give a residue, which was purified by silica gel column chromatography (petroleum ether:acetone=3:1) to give the title compound (2.7 g, 90% purity from ¹H NMR, 31% yield, 98.5% stereopure) as yellow solids. LC-MS (ESI): R_{T}=1.46 min, mass calcd. for C¹¹H₁₆F₃N₃OS 295.1, m/z found 296.1 [M+H]⁺. Chiral analysis (Column: Chiralpak IA 5 μm 4.6*250 mm; Mobile Phase: Hex:EtOH=60:40 at 1 mL/min; Temp: 30° C.; Wavelength: 254 nm, R_{T}=5.382 min). ¹H NMR (400 MHz, CDCl₃) δ 8.89 (s, 1H), 8.73 (s, 1H), 4.79-4.72 (m, 1H), 4.54 (d, J=6.8 Hz, 1H), 1.58 (d, J=6.8 Hz, 3H), 1.26 (s, 9H).

Intermediate 6B-4

(S)-1-(5-(Trifluoromethyl)pyrazin-2-yl)ethan-1-amine hydrochloride

The solution of (S)-2-methyl-N—((S)-1-(5-(trifluoromethyl)pyrazin-2-yl)ethyl)propane-2-sulfinamide 6B-3 (2.7 g, 90% purity, 8.23 mmol) and hydrochloride in 1,4-dioxane (30 mL, 120 mmol) was stirred at 0° C. for 2 hours, then the reaction mixture was concentrated under reduced pressure to give the residue which was triturated with petroleum ether:ethyl acetate=5:1 (20 mL) to give the title compound (2.0 g, 90% purity from ¹HNMR, 96% yield) as yellow solids. ¹H NMR (400 MHz, DMSO-d₆) δ 9.28 (s, 1H), 9.10 (s, 1H), 8.84 (br s, 3H), 4.81 (br s, 1H), 1.60 (d, J=6.8 Hz, 3H).

Intermediate 6B-5

(R)-Methyl 2-hydroxy-3-(((S)-1-(5-(trifluoromethyl)pyrazin-2-yl)ethyl)amino)propanoate

To a solution of (S)-1-(5-(trifluoromethyl)pyrazin-2-yl)ethan-1-amine hydrochloride 6B-4 (1.0 g, 90% purity, 3.95 mmol) in methanol (20 mL) were added (R)-methyl oxirane-2-carboxylate (480 mg, 4.70 mmol) and N-ethyl-N-isopropylpropan-2-amine (2 mL, 12.1 mmol) at room temperature under nitrogen atmosphere. After stirred at 60° C. overnight, the reaction mixture was concentrated under reduced pressure to give a crude compound (1.2 g, 69% purity from LCMS, 71% yield) as yellow oil, which was used directly in the following step without further purification. LC-MS (ESI): R_{T}=1.36 min, mass calcd. for C₁₁H₁₄F₃N₃O₃ 293.1, m/z found 294.1 [M+H]⁺.

Intermediate 6B-6

(R)-2-Hydroxy-N-methyl-3-(((S)-1-(5-(trifluoromethyl)pyrazin-2-yl)ethyl)amino) propanamide

A solution of (R)-methyl 2-hydroxy-3-(((S)-1-(5-(trifluoromethyl)pyrazin-2-yl)ethyl)amino)propanoate 6B-5 (1.2 g, 69% purity, 2.82 mmol) and 2.0 M methylamine in tetrahydrofuran (12 mL, 24 mmol) was stirred at 60° C. in a sealed tube overnight. The reaction mixture was concentrated under reduced pressure and purified by C18 column (acetonitrile:water (0.1% ammonium bicarbonate)=5% to 60%) to give the title compound (500 mg, 90% purity from ¹HNMR, 55% yield, 89% stereopure) as yellow solids. LC-MS (ESI): R_{T}=0.770 min, mass calcd. for C¹¹H₁₅F₃N₄O₂ 292.1, m/z found 293.1 [M+H]⁺. Chiral analysis (Column: Chiralpak AS-H 5 μm 4.6*250 mm; Mobile Phase: Hex: EtOH:DEA=85:15:0.2 at 1 mL/min; Temp: 30° C.; Wavelength: 254 nm, R_{T}=7.474 min).

Intermediate 6B-7

(R)-2-((tert-Butyldimethylsilyl)oxy)-N-methyl-3-(((S)-1-(5-(trifluoromethyl) pyrazin-2-yl)ethyl)amino)propanamide

To a solution of (R)-2-hydroxy-N-methyl-3-(((S)-1-(5-(trifluoromethyl)pyrazin-2-yl)ethyl)amino)propanamide 6B-6 (2.7 g, 90% purity, 8.32 mmol) in dichloromethane (50 mL) were added imidazole (1.8 g, 26.4 mmol) and tert-butylchlorodiphenylsilane (2.5 g, 16.6 mmol) at 0° C. under nitrogen atmosphere. After stirred at room temperature for 8 hours, the reaction mixture was added water (100 mL) and extracted with dichloromethane (50 mL) for three times. The combined organic layers were washed with brine (100 mL), then dried over Na₂SO₄₍ₛ₎, concentrated and purified by silica gel column chromatography (petroleum ether:ethyl acetate=10:1 to 1:1) to give the title compound (3.0 g, 97.4% purity from LCMS, 86% yield, 100% stereopure) as yellow solids. LC-MS (ESI): R_{T}=1.521 min, mass calcd. for C₁₇H₂₉F₃N₄O₂Si 406.2, m/z found 407.2 [M+H]⁺. Chiral analysis (Column: Chiralpak IA 5 μm 4.6*250 mm; Mobile Phase: Hex:EtOH:DEA=85:15:0.2 at 1 mL/min; Temp: 30° C.; Wavelength: 254 nm, R_{T}=4.574 min).

Intermediate 6B-8

(R)-Ethyl 6-methyl-4,5,6,7-tetrahydro-2H-pyrazolo[4,3-c]pyridine-3-carboxylate hydrochloride

The solution of (R)-5-tert-butyl 3-ethyl 6-methyl-6,7-dihydro-2H-pyrazolo[4,3-c]pyridine-3,5(4H)-dicarboxylate (10.0 g, 100% purity, 32.3 mmol) in 4 M hydrochloride in 1,4-dioxane (100 mL) was stirred at room temperature for 2 hours under nitrogen atmosphere. The mixture was concentrated under reduced pressure to give the title compound (8.0 g, 95% purity from ¹H NMR, 96% yield) as yellow solids without further purification. LC-MS (ESI): R_{T}=0.5 min, mass calcd. for C₁₀H₁₅N₃O₂209.1, m/z found 210.2 [M+H]⁺.

Intermediate 6B-9

Ethyl (R)-5-(3,4-dichlorobenzoyl)-6-methyl-4,5,6,7-tetrahydro-2H-pyrazolo[4,3-c]pyridine-3-carboxylate

To a solution of 3,4-dichlorobenzoic acid (5.62 g, 29.4 mmol) and N,N-dimethylformamide (5 drops, 3.35 mmol) in dichloromethane (200 mL) was added oxalyl chloride (3.73 mL, 44.1 mmol) dropwise at 0° C. under nitrogen atmosphere. Then the mixture was warmed to room temperature and stirred for 3 hours. After the reaction was completed, the mixture was concentrated under reduced pressure to give a residue. To a solution of the residue and (R)-ethyl 6-methyl-4,5,6,7-tetrahydro-2H-pyrazolo[4,3-c]pyridine-3-carboxylate hydrochloride 6B-8 (7.15 g, 29.1 mmol) in dichloromethane (200 mL) was added N-ethyl-N-isopropylpropan-2-amine (14.6 mL, 88.3 mmol) dropwise at 0° C., then the mixture was stirred at room temperature overnight. After the reaction was completed, the solvent was removed under reduced pressure to give a brown residue, which was purified by silica column chromatography (petroleum ether:ethyl acetate=1:1) to give the title compound (8.75 g, 98% purity, 76% yield) as white solids.

Intermediate 6B-10

Ethyl (6R)-5-(3,4-dichlorobenzoyl)-6-methyl-1-(tetrahydro-2H-pyran-2-yl)-4,5,6, 7-tetrahydro-1H-pyrazolo[4,3-c]pyridine-3-carboxylate

To a solution of ethyl (R)-5-(3,4-dichlorobenzoyl)-6-methyl-4,5,6,7-tetrahydro-2H-pyrazolo[4,3-c]pyridine-3-carboxylate 6B-9 (69 g, 90% purity, 0.162 mol) in toluene (500 mL) were added 3,4-dihydro-2H-pyran (50 g, 0.594 mol) and 4-methylbenzenesulfonic acid (4 g, 23.0 mmol) at room temperature under nitrogen atmosphere. After stirred at 80° C. for 20 hours, the mixture was concentrated under reduced pressure to give a crude, which was purified by silica gel column chromatography (petroleum ether:ethyl acetate=10:1 to 1:1) to give the crude title compound (80 g, 71% purity from LCMS, 75% yield) as white solids. The crude 6B-10 (25 g, 38.1 mmol) was dissolved in dichloromethane (20 mL) at room temperature. Then the mixture was filtered. The filtrate was concentrated to give the residue, which was purified by silica gel column chromatography (petroleum ether:acetone=10:1 to 6:1) to give the title compound (16.3 g, 91% purity from LCMS) as yellow solids. LC-MS (ESI): R_{T}=1.517 min, mass calcd. for C₂₂H₂₅Cl₂N₃O₄ 465.1, m/z found 466.1 [M+H]⁺.

Intermediate 6B-11

(6R)-5-(tert-Butoxycarbonyl)-6-methyl-2-(tetrahydro-2H-pyran-2-yl)-4,5,6,7-tetrahydro-2H-pyrazolo[4,3-c]pyridine-3-carboxylic acid

To a solution of ethyl (6R)-5-(3,4-dichlorobenzoyl)-6-methyl-1-(tetrahydro-2H-pyran-2-yl)-4,5,6,7-tetrahydro-1H-pyrazolo[4,3-c]pyridine-3-carboxylate 6B-10 (15.0 g, 91% purity, 29.3 mmol) in tetrahydrofuran (100 mL) was added the solution of sodium hydroxide (3.6 g, 90.0 mmol) in water (70 mL) at 0° C. After stirred at 50° C. for 5 hours, the reaction mixture was adjusted pH to 4 with 1 M hydrochloride aqueous solution and extracted with ethyl acetate (30 mL) for three times. The combined organic layers were washed with brine (10 mL), dried over Na₂SO₄₍ₛ₎ and filtered. The filtrate was concentrated by reduced pressure to give the title compound (13.7 g, 85% purity, 91% yield) as yellow solids. LC-MS (ESI): R_{T}=1.27 min, mass calcd. for C₂₀H₂₁Cl₂N₃O₄ 437.1, m/z found 437.9 [M+H]⁺.

Intermediate 6B-12

(6R)—N—((R)-2-((tert-Butyldimethylsilyl)oxy)-3-(methylamino)-3-oxopropyl)-5-(3,4-dichlorobenzoyl)-6-methyl-2-(tetrahydro-2H-pyran-2-yl)-N—((S)-1-(5-(trifluoromethyl)pyrazin-2-yl)ethyl)-4,5,6,7-tetrahydro-2H-pyrazolo[4,3-c]pyridine-3-carboxamide

To a solution of (6R)-5-(tert-butoxycarbonyl)-6-methyl-2-(tetrahydro-2H-pyran-2-yl)-4,5,6,7-tetrahydro-2H-pyrazolo[4,3-c]pyridine-3-carboxylic acid 6B-11 (3.7 g, 85% purity, 7.18 mmol) in N,N-dimethylacetamide (40 mL) was added O-(7-azabenzotriazol-1-yl)-N,N,N′,N′-tetramethyluronium hexafluorophosphate (3.70 g, 9.73 mmol) at 0° C. After stirred at room temperature for 30 minutes, (R)-2-((tert-butyldimethylsilyl)oxy)-N-methyl-3-(((S)-1-(5-(trifluoromethyl)pyrazin-2-yl)ethyl)amino)propanamide 6B-7 (2.0 g, 97.4% purity, 4.78 mmol) and N,N-diisopropylethylamine (4.0 mL, 24.2 mmol) were added to the reaction mixture. Then the reaction mixture was stirred at 20° C. for 15 hours. Another batch of 6B-11 (1.3 g, 97% purity, 2.52 mmol) and O-(7-azabenzotriazol-1-yl)-N,N,N′,N′-tetramethyluronium hexafluorophosphate (900 mg, 2.37 mmol) were added to the reaction mixture. After stirred at 20° C. for 7 hours, the mixture was diluted with water (150 mL) and extracted with ethyl acetate (100 mL) for three times. The combined organic layers were washed with water (100 mL) and brine (100 mL) for three times, dried over Na₂SO₄₍ₛ₎ and filtered. The filtrate was concentrated and purified by silica gel column chromatography (dichloromethane:ethyl acetate=5:1 to 1:1) to give the title compound (4.0 g, 82% purity from LCMS, 76% yield) as yellow solids. LC-MS (ESI): R_{T}=4.02 min, mass calcd. for C₃₇H₄₈Cl₂F₃N₇O₅Si 825.3, m/z found 826.0 [M+H]⁺.

Intermediate 6B-13

(R)-5-(3,4-dichlorobenzoyl)-N—((R)-2-hydroxy-3-(methylamino)-3-oxopropyl)-6-methyl-N—((S)-1-(5-(trifluoromethyl)pyrazin-2-yl)ethyl)-4,5,6,7-tetrahydro-2H-pyrazolo[4,3-c]pyridine-3-carboxamide

To a solution of (6R)—N—((R)-2-((tert-butyldimethylsilyl)oxy)-3-(methylamino)-3-oxopropyl)-5-(3,4-dichlorobenzoyl)-6-methyl-2-(tetrahydro-2H-pyran-2-yl)-N—((S)-1-(5-(trifluoromethyl)pyrazin-2-yl)ethyl)-4,5,6,7-tetrahydro-2H-pyrazolo[4,3-c]pyridine-3-carboxamide 6B-12 (4.0 g, 82% purity, 3.97 mmol) in dichloromethane (120 mL) was added trifluoroacetic acid (40 mL) at 0° C. under nitrogen atmosphere. After stirred at 0° C. for 5 hours, the reaction mixture was added ice water (150 mL). The organic phase was separated and washed with saturated sodium bicarbonate aqueous solution (150 mL) and brine (150 mL), dried over Na₂SO₄₍ₛ₎, filtered. The filtrate was concentrated to give a residue which was purified by C18 column (acetonitrile:water (0.1% ammonium bicarbonate)=5% to 60%) to give the title compound (2.3 g, 98% purity from LCMS, 90% yield, 92.5% stereopure) as yellow solids. LC-MS (ESI): R_{T}=2.66 min, mass calcd. for C₂₆H₂₆Cl₂F₃N₇O₄ 627.1, m/z found 627.8 [M+H]⁺. Chiral analysis (Column: Chiralpak TB N-5 5 μm 4.6*250 mm; Mobile Phase: Hexane:EtOH:DEA=70:30:0.2 at 1 mL/min; Temp: 30° C.; Wavelength: 254 nm, R_{T}=8.911 min).

Compound 6B

(3R,7S)-2-(3,4-dichlorobenzoyl)-N,3-dimethyl-10-oxo-9-((S)-1-(5-(trifluoromethyl) pyrazin-2-yl)ethyl)-1,2,3,4,7,8,9,10-octahydropyrido[4′,3′:3,4]pyrazolo[1,5-a]pyrazine-7-carboxamide

To a solution of (R)-5-(3,4-dichlorobenzoyl)-N—((R)-2-hydroxy-3-(methylamino)-3-oxopropyl)-6-methyl-N—((S)-1-(5-(trifluoromethyl)pyrazin-2-yl)ethyl)-4,5,6,7-tetrahydro-2H-pyrazolo[4,3-c]pyridine-3-carboxamide 6B-13 (2.6 g, 98% purity, 4.06 mmol) in tetrahydrofuran (52 mL) were added tributylphosphine (1.3 g, 6.43 mmol) and (E)-diisopropyl diazene-1,2-dicarboxylate (1.3 g, 6.43 mmol) at −10° C. under nitrogen atmosphere. After stirred at −10° C. for 1 hour, the mixture was quenched with saturated ammonium chloride aqueous solution (50 mL) at −10° C. The mixture was extracted with ethyl acetate (50 mL) for three times. The combined organic layers were washed with brine (100 mL), dried over Na₂SO₄₍ₛ₎ and filtered. The filtrate was concentrated and purified by silica gel column chromatography (petroleum ether:acetone=10:1 to 2:1) to give the title compound (1.8 g, 92% purity from LCMS, 88% yield, 96.3% stereopure) as white solids. LC-MS (ESI): R_{T}=2.81 min, mass calcd. for C₂₆H₂₄Cl₂F₃N₇O₃ 609.1, m/z found 609.8 [M+H]⁺. Chiral analysis (Column: Chiralpak IC 5 μm 4.6*250 mm; Mobile Phase: ACN=100 at 1 mL/min; Temp: 30° C.; Wavelength: 254 nm, R_{T}=12.684 min). The 6B crude (2.6 g, 89% purity, 3.79 mmol) was further separated by chiral HPLC (separation condition: Column Chiralpak IC 5 μm*30*250 mm; Mobile Phase: MeCN=100 at 20 mL/min; Col. Temp 30° C.; Wavelength 254 nm) to afford the title compound (2.0 g, 99.5% purity from LCMS, 86.3% yield, 99.9% stereopure) as white solids. LC-MS (ESI): R_{T}=8.682 min, mass calcd. for C₂₆H₂₄Cl₂F₃N₇O₃ 609.1, m/z found 610.0 [M+H]⁺. Chiral analysis (Column: Chiralpak IC 5 μm 4.6*250 mm; Mobile Phase: ACN=100 at 1 mL/min; Temp: 30° C.; Wavelength: 254 nm, R_{T} 12.496 min). H NMR (400 MHz, DMSO-d₆) δ 9.15 (s, 1H), 8.99-8.82 (m, 1H), 8.10 (s, 1H), 7.74-7.72 (m, 2H), 7.45-7.42 (m, 1H), 5.97-5.68 (m, 1H), 5.46-5.01 (m, 2H), 4.65-4.06 (m, 2H), 3.93 (s, 2H), 2.98-2.84 (m, 1H), 2.65 (d, J=4.4 Hz, 3H), 2.61-2.52 (m, 1H), 1.55 (br s, 3H), 1.27-1.11 (m, 3H). ¹⁹F NMR (376 MHz, DMSO-d₆) δ −66.14.

Compounds 7A and 7B

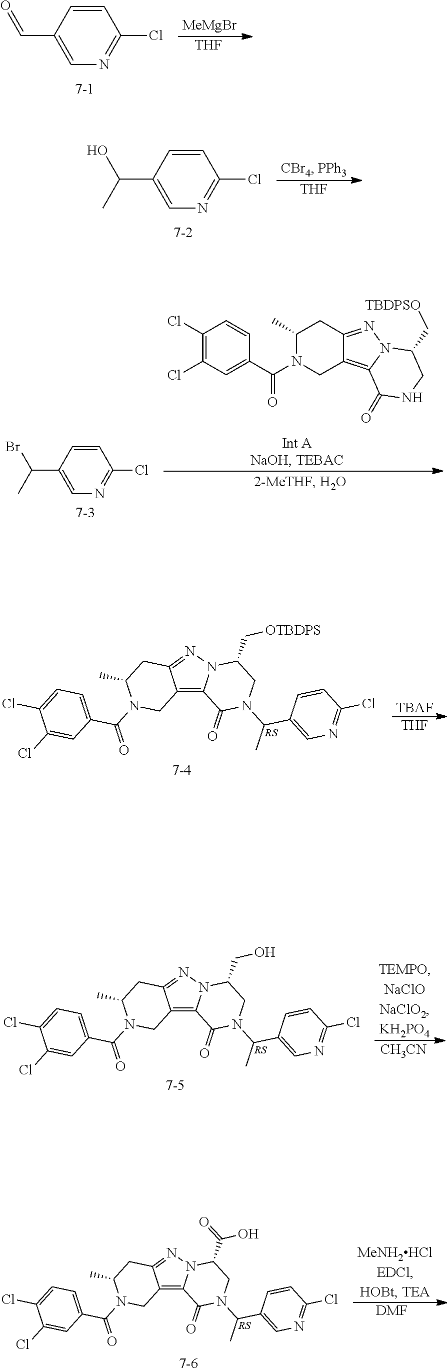

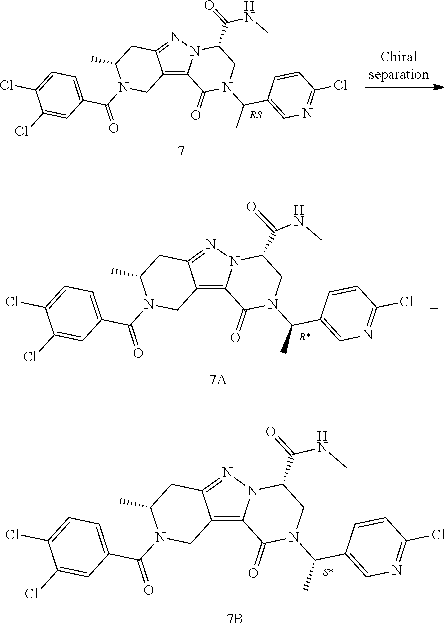

Intermediate 7-2

1-(6-Chloropyridin-3-yl)ethanol

To the solution of 6-chloronicotinaldehyde 7-1 (1.8 g, 12.7 mmol) in tetrahydrofuran (20 mL) was added 1 M methylmagnesium bromide in tetrahydrofuran (25 mL, 25 mmol) at 0° C. The mixture was stirred at 0° C. for 1 hour. The mixture was quenched with saturated aqueous ammonium chloride solution (30 mL) and extracted with ethyl acetate (30 mL) twice. The combined organic layers were washed with brine (40 mL), dried over Na₂SO₄₍ₛ₎ and filtered. The filtrate was concentrated to give the title compound (1.8 g, 90% purity, 80.8% yield) as a yellow oil. ¹H NMR (400 MHz, CDCl₃) δ 8.37 (d, J=6.4 Hz, 1H), 7.71 (dd, J=8.4 and 2.4 Hz, 1H), 7.33 (d, J=8.0 Hz, 1H), 5.03-4.94 (m, 1H), 1.97 (d, J=7.6 Hz, 1H), 1.52 (d, J=5.6 Hz, 3H).

Intermediate 7-3

5-(1-Bromoethyl)-2-chloropyridine

To a solution of 1-(6-chloropyridin-3-yl)ethanol 7-2 (1.6 g, 90% purity, 9.14 mmol) in tetrahydrofuran (20 mL) were added triphenylphosphine (4.8 g, 18.3 mmol) and perbromomethane (4.3 g, 13.0 mmol) at 0° C. After being stirred at 25° C. for 4 hours, the mixture was filtered. The filtrate was concentrated and purified by column chromatography on silica gel (petroleum ether:ethyl acetate=5:1) to give the title compound (1.6, 90% purity from ¹H NMR, 62.3% yield) as a yellow oil. LC-MS (ESI): R_{T}=1.59 min, mass calcd. for C₇H₇BrClN 219.0 found 219.9 [M+H]⁺. ¹H NMR (400 MHz, CDCl₃) δ 8.42 (d, J=2.8 Hz, 1H), 7.77 (dd, J=8.0 and 2.4 Hz, 1H), 7.33 (d, J=8.4 Hz, 1H), 5.16 (q, J=6.8 Hz, 1H), 2.04 (d, J=6.8 Hz, 3H).

Intermediate 7-4

(3R,7S)-7-(((tert-Butyldiphenylsilyl)oxy)methyl)-9-(1-(6-chloropyridin-3-yl)ethyl)-2-(3,4-dichlorobenzoyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (600 mg, 90% purity, 0.834 mmol) in 2-Methyltetrahydrofuran (6 mL) and 50% wt. sodium hydroxide in water (6 mL, 150.0 mmol) was added 5-(1-bromoethyl)-2-chloropyridine 7-3 (320 mg, 90% purity, 1.3 mmol) and N-benzyl-N,N-diethylethanaminium chloride (27 mg, 0.119 mmol) at room temperature. The mixture was stirred at room temperature for 2 hours. The reaction mixture was quenched with water (20 mL) and extracted with ethyl acetate (20 mL) twice. The combined organic layers were washed with brine (30 mL), dried over Na₂SO₄₍ₛ₎ and filtered. The filtrate was concentrated and purified by C18 column (acetonitrile: water=50% to 60%) to give the title compound (600 mg, 51% purity, 46.6% yield) as a white solid. LC-MS (ESI): R_{T}=1.98 min, mass calcd. for C₄₁H₄₂C₁₃N₅O₃Si 785.2 found 786.4 [M+H]⁺.

Intermediate 7-5

(3R,7S)-9-(1-(6-Chloropyridin-3-yl)ethyl)-2-(3,4-dichlorobenzoyl)-7-(hydroxymethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-9-(1-(6-chloropyridin-3-yl)ethyl)-2-(3,4-dichlorobenzoyl)-3-methyl-1,2,3,4,8,9-hexahydropyrido[4′,3′:3,4]pyrazolo[1,5-a]pyrazin-10(7H)-one 7-4 (600 mg, 51% purity, 0.389 mmol) in tetrahydrofuran (6 mL) was added 1 M tetrabutylammonium fluoride in tetrahydrofuran (0.8 mL, 0.8 mmol) at room temperature. After being stirred at room temperature for 1 hour, the reaction mixture was quenched with water (10 mL) and extracted with ethyl acetate (10 mL) twice. The combined organic layers were washed with brine (20 mL) twice, dried over Na₂SO₄₍ₛ₎ and filtered. The filtrate was concentrated and purified by column chromatography on silica gel (dichloromethane:ethyl acetate=10:1) to give desired compound (200 mg, 100% purity, 93.7% yield) as a white solid. LC-MS (ESI): R_{T}=1.56 min, mass calcd. for C₂₅H₂₄C₁₃N₅O₃ 547.1 m/z found 548.2 [M+H]⁺.

Intermediate 7-6

(3R,7S)-9-(1-(6-Chloropyridin-3-yl)ethyl)-2-(3,4-dichlorobenzoyl)-3-methyl-10-oxo-1,2,3,4,7,8,9,10-octahydropyrido[4′,3′:3,4]pyrazolo[1,5-a]pyrazine-7-carboxylic acid

To a solution of (3R,7S)-9-(1-(6-chloropyridin-3-yl)ethyl)-2-(3,4-dichlorobenzoyl)-7-(hydroxymethyl)-3-methyl-1,2,3,4,8,9-hexahydropyrido[4′,3′:3,4]pyrazolo[1,5-a]pyrazin-10(7H)-one 7-5 (200 mg, 100% purity, 0.364 mmol), sodium chlorite (80 mg, 80% purity, 0.708 mmol) and 2,2,6,6-tetramethylpiperidinooxy (115 mg, 0.736 mmol) in acetonitrile (2 mL) was added saturated aqueous potassium phosphate monobasic solution (2 mL) and 10% sodium hypochlorite solution (1 mL, 1.68 mmol) at 0° C. After being stirred at room temperature overnight, the reaction mixture was quenched with saturated aqueous sodium sulfite solution (10 mL), acidized with 1N hydrochloride to pH˜4, and extracted with ethyl acetate (10 mL) twice. The combined organic layers were dried over Na₂SO₄₍ₛ₎, and filtered. The filtrate was concentrated, and purified by C18 column (acetonitrile:water=50% to 60%) to give the title compound (170 mg, 100% purity from LCMS, 82.9% yield) as a white solid. LC-MS (ESI): R_{T}=1.28 min, mass calcd. for C₂₅H₂₂C₁₃N₅O₄ 561.1/z found 562.1[M+H]⁺.

Compound 7

(3R,7S)-9-(1-(6-Chloropyridin-3-yl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide

To a solution of (3R,7S)-9-(1-(6-chloropyridin-3-yl)ethyl)-2-(3,4-dichlorobenzoyl)-3-methyl-10-oxo-1,2,3,4,7,8,9,10-octahydropyrido[4′,3′:3,4]pyrazolo[1,5-a]pyrazine-7-carboxylic acid 7-6 (170 mg 100%, purity, 0.302 mmol), N1-((ethylimino)methylene)-N3,N3-dimethylpropane-1,3-diamine hydrochloride (117 mg, 0.61 mmol), methylamine hydrochloride (50 mg, 0.741 mmol) and 1H-benzo[d][1,2,3]triazol-1-ol (85 mg, 0.629 mmol) in N,N-dimethylformamide (3 mL) was added triethylamine (0.35 mL, 1.97 mmol) at 0° C. After being stirred at room temperature under nitrogen atmosphere for 3 hours, the mixture was quenched with saturated aqueous ammonium chloride solution (10 mL) and extracted with ethyl acetate (10 mL) twice. The combined organic layers were washed with brine (20 mL), dried over Na₂SO₄₍ₛ₎ and filtered. Which was purified by C18 column (acetonitrile: water (0.1% ammonium bicarbonate)=45% to 55%) to give the title compound (110 mg, 100% purity, 63.2% yield) as a yellow solid. LC-MS (ESI): R_{T}=2.60 min and 2.68 min, mass calcd. for C₂₆H₂₅C₁₃N₆O₃ 574.1, m/z found 575.2 [M+H]⁺.

Compounds 7A and 7B

(3R,7S)-9-((R*)-1-(6-Chloropyridin-3-yl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide (7A), and (3R,7S)-9-((S*)-1-(6-Chloropyridin-3-yl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide (7B)

The racemate of (3R,7S)-9-(1-(6-chloropyridin-3-yl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide compound 7 (140 mg, 100% purity, 0.243 mmol) was separated by chiral prep. HPLC (Column: Chiralpak IE 5 μm 30*250 mm; Mobile Phase: ACN:IPA=70:30 at 30 mL/min; Col. Temp: 30° C.; Wavelength: 254 nm) to give the compound 7A (30.6 mg, 99.6% purity, 21.8% yield, 100% stereopure) as a white solid and compound 7B (19.0 mg, 99.7% purity, 13.5% yield, 100% stereopure) as a white solid.

Compound 7A

LC-MS (ESI): R_{T}=3.546 min, mass calcd. for C₂₆H₂₅C₁₃N₆O₃ 574.1, m/z found 575.2 [M+H]⁺. Chiral analysis (Column: Chiralpak IE 5 μm 4.6*250 mm; Mobile Phase: ACN:IPA=70:30 at 1 mL/min; Temp: 30° C.; Wavelength: 254 nm; R_{T}=5.415 min). ¹H NMR (400 MHz, CDCl₃) δ 8.36-8.31 (m, 1H), 7.62-7.58 (m, 1H), 7.53-7.51 (m, 2H), 7.32-7.30 (m, 1H), 7.27-7.26 (m, 1H), 7.26-7.25 (m, 1H), 5.98-5.31 (m, 3H), 4.92-4.83 (m, 1H), 4.58-4.36 (m, 1H), 4.00-3.85 (m, 2H), 3.13-2.97 (m, 1H), 2.76-2.66 (m, 4H), 1.63-1.61 (m, 3H), 1.30 (d, J=6.8 Hz, 3H).

Compound 7B

LC-MS (ESI): R_{T}=3.635 min, mass calcd. for C₂₆H₂₅C₁₃N₆O₃ 574.1, m/z found 575.2 [M+H]⁺. Chiral analysis (Column: Chiralpak IE 5 μm 4.6*250 mm; Mobile Phase: ACN:IPA=70:30 at 1 mL/min; Temp: 30° C.; Wavelength: 254 nm; R_{T}=7.199 min). ¹H NMR (400 MHz, CDCl₃) −δ 8.44-8.36 (m, 1H), 7.72-7.61 (m, 1H), 7.55-7.50 (m, 2H), 7.35-7.31 (m, 1H), 7.29-7.27 (m, 1H), 7.25-7.23 (m, 1H), 6.25-5.97 (m, 2H), 4.92-4.42 (m, 3H), 4.21-4.11 (m, 1H), 3.52-3.39 (m, 1H), 3.14-2.97 (m, 1H), 2.83-2.81 (m, 3H), 2.73-2.69 (m, 1H), 1.69-1.59 (m, 3H), 1.29 (d, J=6.8 Hz, 3H).

Compounds 8A and 8B

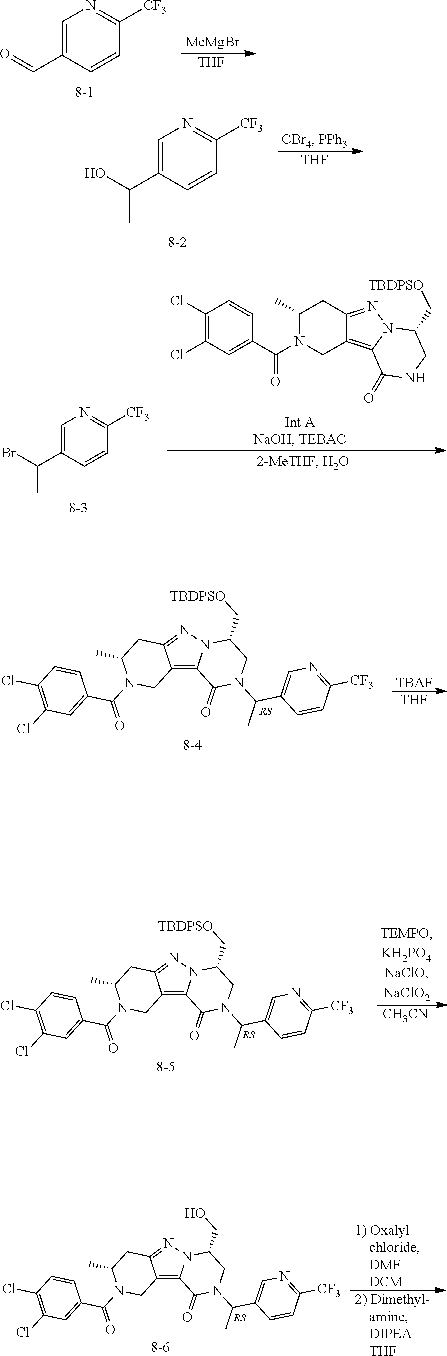

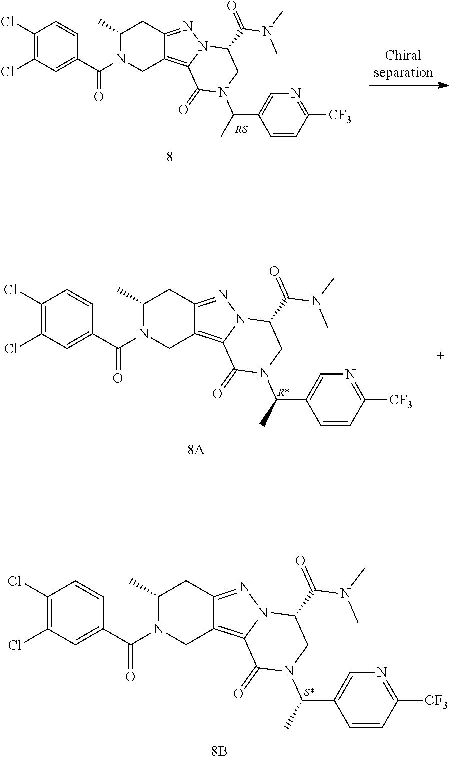

Intermediate 8-2

1-(6-(Trifluoromethyl)pyridin-3-yl)ethanol

To a solution of 6-(trifluoromethyl)nicotinaldehyde (900 mg, 5.14 mmol) in tetrahydrofuran (10 mL) was added dropwise 3 M methylmagnesium bromide in tetrahydrofuran (2.7 mL, 8.10 mmol) at −70° C. for 2 hours. The reaction mixture was poured into saturated ammonium chloride aqueous solution (40 mL) and extracted with ethyl acetate (20 mL) twice. The combined organic layers were washed with brine (20 mL), dried over Na₂SO₄₍ₛ₎ and filtered. The filtrate was concentrated to give the title compound (900 mg, 90% purity from ¹H NMR, 82% yield) as a colorless oil. ¹H NMR (400 MHz, CDCl₃) δ 8.68 (br s, 1H), 7.93-7.91 (m, 1H), 7.67 (d, J=8.0 Hz, 1H), 5.07-5.02 (m, 1H), 2.64 (br s, 1H), 1.55 (d, J=6.4 Hz, 3H).

Intermediate 8-3

5-(1-Bromoethyl)-2-(trifluoromethyl)pyridine

To a solution of 1-(6-(Trifluoromethyl)pyridin-3-yl)ethanol 8-2 (900 mg, 90% purity, 4.24 mmol) in tetrahydrofuran (15 mL) were added triphenylphosphine (2.0 g, 7.63 mmol) and perbromomethane (2.1 g, 6.33 mmol) at 0° C. After being stirred at 25° C. for 0.5 hour, the mixture was filtered. The filtrate was concentrated under reduced pressure to give the residue, which was purified by silica gel column chromatography (petroleum ether:ethyl acetate=100:1) to give the title compound (1.0 g, 90% purity by ¹H NMR, 84% yield) as a colorless oil. ¹H NMR (400 MHz, CDCl₃) δ 8.77 (br s, 1H), 7.98-7.95 (m, 1H), 7.68 (d, J=8.0, 1H), 5.24-5.18 (m, 1H), 2.80 (d, J=6.8 Hz, 3H).

Intermediate 8-4

(3R,7S)-7-(((tert-Butyldiphenylsilyl)oxy)methyl)-2-(3,4-dichlorobenzoyl)-3-methyl-9-(1-(6-(trifluoromethyl)pyridin-3-yl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (500 mg, 100% purity, 0.77 mmol) and 5-(1-bromoethyl)-2-(trifluoromethyl)pyridine 8-3 (400 mg, 100% purity, 1.58 mmol) in 2-methyltetrahydrofuran (2.5 mL) was added 50% wt. Sodium hydroxide in water (2.5 mL) and N-benzyl-N,N-diethylethanaminium chloride (25 mg, 0.11 mmol) slowly at room temperature. After being stirred at room temperature for 3 hours, the mixture was diluted with water (10 mL) and concentrated at room temperature under reduced pressure to remove the volatile. The remained aqueous layer was acidified with 1 M hydrochloride aqueous solution (30 mL) and extracted with ethyl acetate (60 mL) twice. The combined organic layers were washed with brine (50 mL), dried over Na₂SO₄₍ₛ₎ and filtered. The filtrate was concentrated to give the crude compound (730 mg, 84% purity from LCMS, 96.8% yield) as yellow solids. LC-MS (ESI): R_{T}=2.33 min, mass calcd. for C₄₂H₄₂Cl₂F₃N₅O₃Si 819.2, m/z found 820.2 [M+H]⁺.

Intermediate 8-5

(3R,7S)-2-(3,4-Dichlorobenzoyl)-7-(hydroxymethyl)-3-methyl-9-(1-(6-(trifluoromethyl)pyridin-3-yl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-9-(1-(6-(trifluoromethyl)pyridin-3-yl)ethyl)-1,2,3,4,8,9-hexahydropyrido[4′,3′:3,4]pyrazolo[1,5-a]pyrazin-10(7H)-one 8-4 (730 mg, 84% purity, 0.75 mmol) in tetrahydrofuran (10 mL) was added 1 M tetrabutylammonium fluoride in tetrahydrofuran (1 mL, 1 mmol) at 0° C. After being stirred at 0° C. for 1 hour, the mixture was concentrated under reduced pressure to give crude. The crude was purified by silica gel column chromatography (dichloromethane:methanol=10:1) to give the title compound (430 mg, 100% purity from LCMS, 98.8% yield) as yellow solids. LC-MS (ESI): R_{T}=1.59 min, mass calcd. for C₂₆H₂₄Cl₂F₃N₅O₃ 581.1, m/z found 582.0 [M+H]⁺.

Intermediate 8-6

(3R,7S)-2-(3,4-Dichlorobenzoyl)-3-methyl-10-oxo-9-(1-(6-(trifluoromethyl)pyridin-3-yl)ethyl)-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-7-(hydroxymethyl)-3-methyl-9-(1-(6-(trifluoromethyl)pyridin-3-yl)ethyl)-1,2,3,4,8,9-hexahydropyrido[4′,3′:3,4]pyrazolo[1,5-a]pyrazin-10(7H)-one 8-5 (430 mg, 100% purity, 0.74 mmol) in acetonitrile (3 mL) was added 2,2,6,6-tetramethylpiperidinooxy (240 mg, 1.54 mmol), sodium chlorite (160 mg, 80% purity, 1.42 mmol), saturated potassium dihydrogenphosphate aqueous solution (3 mL) and sodium hypochlorite (0.9 mL, 1.51 mmol) at 0° C. After being stirred at 0° C. overnight, the mixture was diluted with sodium sulfite saturated solution (10 mL) and extracted with ethyl acetate (50 mL) twice. The combined organic layers were washed with brine (30 mL), dried over Na₂SO₄₍ₛ₎ and filtered. The filtrate was concentrated and purified by C18 column (acetonitrile:water=5% to 100%) to give the title compound (390 mg, 100% purity from LCMS, 88.6% yield) as yellow solids. LC-MS (ESI): R_{T}=1.27 min and 1.29 min, mass calcd. for C₂₆H₂₂Cl₂F₃N₅O₄ 595.1, m/z found 596.0 [M+H]⁺.

Compound 8

(3R,7S)-2-(3,4-Dichlorobenzoyl)-N,N,3-trimethyl-10-oxo-9-(1-(6-(trifluoromethyl)pyridin-3-yl)ethyl)-1,2,3,4,7,8,9,10-octahydropyrido[4′,3′:3,4]pyrazolo[1,5-a]pyrazine-7-carboxamide

To a solution of (3R,7S)-2-(3,4-Dichlorobenzoyl)-3-methyl-10-oxo-9-(1-(6-(trifluoromethyl)pyridin-3-yl)ethyl)-1,2,3,4,7,8,9,10-octahydropyrido[4′,3′:3,4]pyrazolo[1,5-a]pyrazine-7-carboxylic acid 8-6 (270 mg, 89% purity, 0.403 mmol) in dichloromethane (5 mL) was added oxalyl chloride (173 mg, 1.36 mmol) and N,N-dimethylformamide (0.08 mL) at 0° C. under nitrogen atmosphere. After being stirred at 0° C. for 1 hour, the reaction mixture was concentrated to give a crude product, which was added to a mixture of 1 M dimethylamine in tetrahydrofuran (0.7 mL, 1.4 mmol) and N-ethyl-N-isopropylpropan-2-amine (176 mg, 1.36 mmol) in dichloromethane (5 mL). After being stirred at 0° C. for 1 hour, the mixture was diluted with water (20 mL) and extracted with ethyl acetate (20 mL) twice. The combined organic layers were washed with water (30 mL) for three times and brine (20 mL), dried over Na₂SO₄₍ₛ₎ and filtered. The filtrate was concentrated under reduced pressure to give a residue, which was purified by C18 (acetonitrile:water=40% to 65%) and silica gel column chromatography (dichloromethane:methanol 100:1 to 10:1) to give the title compound (180 mg, 98% purity from LCMS, 70% yield) as an off-white solid. LC-MS (ESI): R_{T}=1.60 min, mass calcd. for C₂₈H₂₇Cl₂F₃N₆O₃ 622.2, m/z found 623.2 [M+H]⁺.

Compounds 8A and 8B

(3R,7S)-2-(3,4-dichlorobenzoyl)-N,N,3-trimethyl-10-oxo-9-((R*)-1-(6-(trifluoromethyl)pyridin-3-yl)ethyl)-1,2,3,4,7,8,9,10-octahydropyrido[4′,3′:3,4]pyrazolo[1,5-a]pyrazine-7-carboxamide (8A), and (3R,7S)-2-(3,4-dichlorobenzoyl)-N,N,3-trimethyl-10-oxo-9-((S*)-1-(6-(trifluoromethyl)pyridin-3-yl)ethyl)-1,2,3,4,7,8,9,10-octahydropyrido[4′,3′:3,4]pyrazolo[1,5-a]pyrazine-7-carboxamide (8B)

A racemic of (3R,7S)-2-(3,4-Dichlorobenzoyl)-N,N,3-trimethyl-10-oxo-9-(1-(6-(trifluoromethyl)pyridin-3-yl)ethyl)-1,2,3,4,7,8,9,10-octahydropyrido[4′,3′:3,4]pyrazolo[1,5-a]pyrazine-7-carboxamide 8 (180 mg, 98% purity, 0.283 mmol) was separated by chiral prep. HPLC (separation condition: Column: Chiralpak IE 5 μm 20*250 mm; Mobile Phase: Hex:EtOH=30:70 at 18 mL min; Temp: 30° C.; Wavelength: 254 nm) to afford compound 8A (18.8 mg, 99% purity from LCMS, 10% yield, 100% stereopure) and compound 8B (53.8 mg, 99% purity from LCMS, 30% yield, 100% stereopure) as an off-white solid.

Compound 8A

LC-MS (ESI): R_{T}=4.081 min, mass calcd. for C₂₈H₂₇Cl₂F₃N₆O₃ 622.2, m/z found 623.3 [M+H]⁺. Chiral analysis (Column: Chiralpak IE 5 μm 4.6*250 mm; Mobile Phase: Hex:EtOH=30:70 at 1 mL/min; Temp: 30° C.; Wavelength: 254 nm, R_{T}=8.426 min). ¹H NMR (400 MHz, CDCl₃) δ 8.68 (s, 1H), 7.84 (d, J=8.0 Hz, 1H), 7.67 (d, J=8.4 Hz, 1H), 7.54-7.50 (m, 2H), 7.28-7.25 (m, 1H), 6.22-6.06 (m, 1H), 5.78-5.18 (m, 2H), 4.85-4.30 (m, 2H), 3.98-3.95 (m, 1H), 3.27-3.24 (m, 1H), 3.10-2.91 (m, 1H), 2.77 (s, 3H), 2.69-2.67 (m, 4H), 1.64 (d, J=6.4 Hz, 3H), 1.30 (d, J=5.2 Hz, 3H). ¹⁹F NMR (376 MHz, CDCl₃) δ −67.92.

Compound 8B

LC-MS (ESI): R_{T}=4.214 min, mass calcd. for C₂₈H₂₇Cl₂F₃N₆O₃ 622.2, m/z found 623.3 [M+H]⁺. Chiral analysis (Column: Chiralpak IE 5 μm 4.6*250 mm; Mobile Phase: Hex:EtOH=30:70 at 1 mL/min; Temp: 30° C.; Wavelength: 254 nm, R_{T}=10.671 min). ¹H NMR (400 MHz, CDCl₃) δ 8.71 (s, 1H), 7.86-7.85 (m, 1H), 7.69 (d, J=8.4 Hz, 1H), 7.54-7.50 (m, 2H), 7.28-7.25 (m, 1H), 6.18-6.04 (m, 1H), 5.78-5.16 (m, 2H), 4.86-4.27 (m, 2H), 3.88-3.83 (m, 1H), 3.39-3.37 (m, 1H), 3.16 (s, 3H), 3.04-2.95 (m, 4H), 2.70-2.65 (m, 1H), 1.63 (d, J=6.8 Hz, 3H), 1.27-1.25 (m, 3H). ¹⁹F NMR (376 MHz, CDCl₃) δ −67.93.

Compounds 9A and 9B

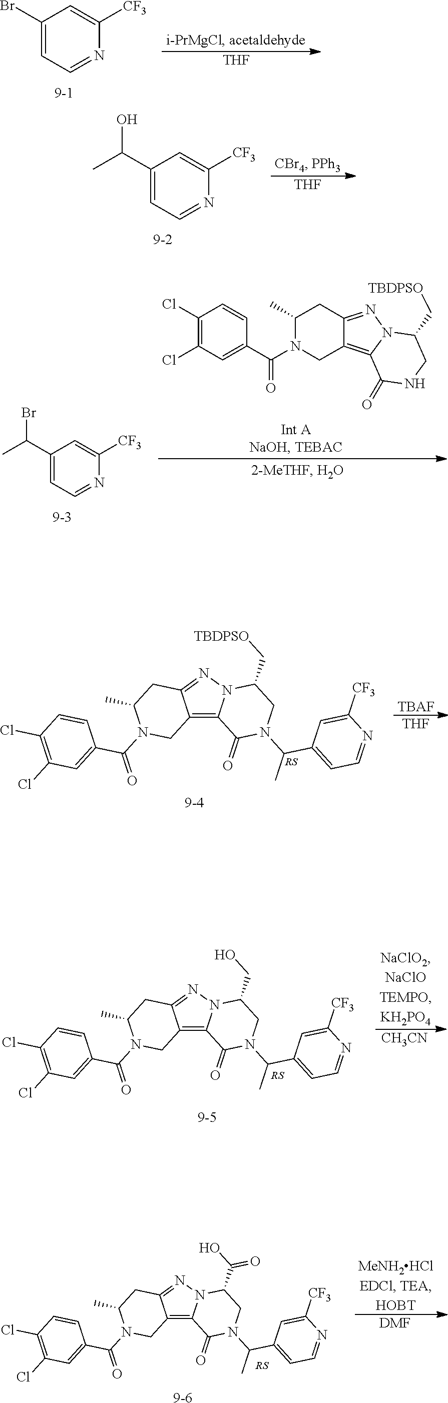

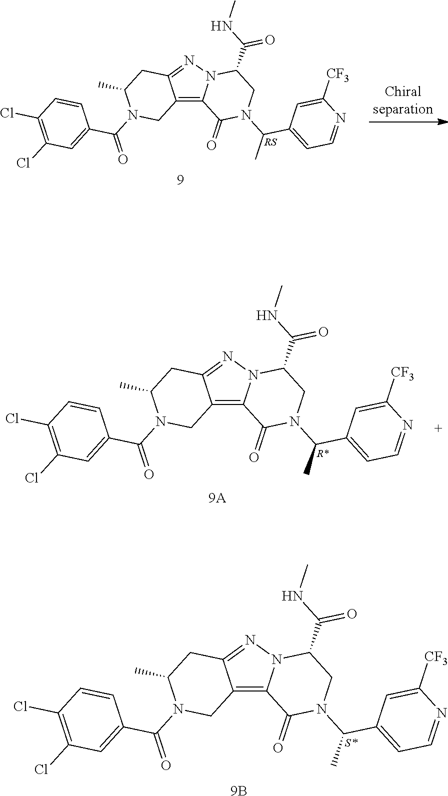

Intermediate 9-2

1-(2-(Trifluoromethyl)pyridin-4-yl)ethanol

To the solution of 4-bromo-2-(trifluoromethyl)pyridine 9-1 (500 mg, 2.21 mmol) in tetrahydrofuran (15 mL) at 0° C. was slowly added isopropylmagnesium chloride (2 mL, 2.60 mmol). The reaction mixture was stirred at room temperature for 30 minutes then acetaldehyde (0.2 ml, 3.56 mmol) was added and the resulting mixture was stirred at 0° C. for 1 hour. It was poured into saturated ammonium chloride aqueous solution (20 mL) and extracted with ethyl acetate (50 mL) for three times. The combined organic layers were washed with brine (20 mL), dried over Na₂SO₄₍ₛ₎ and filtered. The filtrate was concentrated and the residue was purified by C18 (acetonitrile:water (0.1% ammonium bicarbonate)=30% to 80%) to give the title compound (300 mg, 73% purity from LCMS, 52% yield) as white solids. LC-MS (ESI): R_{T}=1.33 min, mass calcd. for C₈H₈F₃NO 191.1 m/z found 192.1[M+H]⁺. ¹H NMR (400 MHz, DMSO-d₆) δ 8.72 (d, J=5.2 Hz, 1H), 7.86 (s, 1H), 7.69 (d, J=4.8 Hz, 1H), 5.64 (br, s, 1H), 4.90 (q, J=6.4 Hz, 1H), 1.39 (d, J=6.8 Hz, 3H).

Intermediate 9-3

4-(1-Bromoethyl)-2-(trifluoromethyl)pyridine

To the solution of 1-(2-(trifluoromethyl)pyridin-4-yl)ethanol 9-2 (1.7 g, 73% purity, 6.49 mmol) in tetrahydrofuran (50 mL) at 0° C. was slowly added perbromomethane (4.3 g, 12.9 mmol). The reaction mixture was stirred at room temperature. Then triphenylphosphine (3.4 g, 12.9 mmol) was added and the reaction mixture was stirred at room temperature for 2 hours. The reaction mixture was filtered. The filtrate was concentrated and the residue was purified by silica gel column chromatography (petroleum ether:ethyl acetate=20:1 to 10:1) to give the title compound (1.3 g, 90% purity from NMR, 71% yield) as white solids. ¹H NMR (400 MHz, CDCl₃) δ 8.71 (d, J=5.2 Hz, 1H), 7.73 (s, 1H), 7.54 (d, J=5.2 Hz, 1H), 5.12 (q, J=6.8 Hz, 1H), 2.05 (d, J=6.8 Hz, 3H).

Intermediate 9-4

(3R,7S)-7-(((tert-Butyldiphenylsilyl)oxy)methyl)-2-(3,4-dichlorobenzoyl)-3-methyl-9-(1-(2-(trifluoromethyl)pyridin-4-yl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (800 mg, 100% purity, 1.24 mmol), 4-(1-bromoethyl)-2-(trifluoromethyl)pyridine 9-3 (520 mg, 90% purity, 1.84 mmol) and N-benzyl-N,N-diethylethanaminium chloride (45 mg, 0.20 mmol) in 2-methyltetrahydrofuran (8 mL) and 50% wt. sodium hydroxide solution (4 mL) at 0° C. Then the reaction mixture was stirred at room temperature for 2 hours. The reaction mixture was quenched with water (20 mL) and extracted with ethyl acetate (20 mL) twice. The combined organic layers were dried over Na₂SO₄₍ₛ₎ and filtered. The filtrate was concentrated under reduced pressure to give crude product, which was purified by C18 column (acetonitrile:water=5% to 90%) to give the title compound (960 mg, 100% purity from LCMS, 95% yield) as a white solid. LC-MS (ESI): R_{T}=1.25 min, mass calcd. for C₄₂H₄₂Cl₂F₃N₅O₃Si 819.2 m/z found 820.2 [M+H]⁺.

Intermediate 9-5

(3R,7S)-2-(3,4-Dichlorobenzoyl)-7-(hydroxymethyl)-3-methyl-9-(1-(2-(trifluoromethyl)pyridin-4-yl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-9-(1-(2-(trifluoromethyl)pyridin-4-yl)ethyl)-1,2,3,4,8,9-hexahydropyrido[4′,3′:3,4]pyrazolo[1,5-a]pyrazin-10(7H)-one 9-4 (960 mg, 100% purity, 1.17 mmol) in tetrahydrofuran (9 mL) was added 1 M tetrabutylammonium fluoride in tetrahydrofuran (2.5 mL, 2.5 mmol) at room temperature, then the mixture was stirred at room temperature for 3 hours. The reaction mixture was poured into ethyl acetate (30 mL) and washed with water (20 mL) three times. The combined organic layers were dried over Na₂SO₄₍ₛ₎ and filtered. The filtrate was concentrated under reduced pressure to give the residue, which was purified by column gel column chromatography (dichloromethane:methanol=40:1) to give the title compound (595 mg, 99% purity from LCMS, 86% yield) as a white solid. LC-MS (ESI): R_{T}=0.736 min, mass calcd. for C₂₆H₂₄Cl₂F₃N₅O₃ 581.1 m/z found 582.0 [M+H]⁺.

Intermediate 9-6

(3R,7S)-2-(3,4-Dichlorobenzoyl)-3-methyl-10-oxo-9-(1-(2-(trifluoromethyl)pyridin-4-yl)ethyl)-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-7-(hydroxymethyl)-3-methyl-9-(1-(2-(trifluoromethyl)pyridin-4-yl)ethyl)-1,2,3,4,8,9-hexahydropyrido[4′,3′:3,4]pyrazolo[1,5-a]pyrazin-10(7H)-one 9-5 (595 mg, 99% purity, 1.01 mmol) in acetonitrile (6 mL) was added saturated aqueous potassium dihydrogen phosphate solution (9 mL), 2,2,6,6-tetramethylpiperidinooxy (320 mg, 2.05 mmol), sodium chlorite (230 mg, purity 80%, 2.03 mmol), 5.5% sodium hypochlorite solution (1.2 mL, 2.02 mmol) at 0° C. The reaction mixture was allowed to slowly return to room temperature. After being stirred at room temperature for 5 hours, the reaction mixture was quenched with saturated sodium thiosulfate aqueous solution (5 mL), acidified with 1 M hydrochloric acid solution to pH=4˜5, and extracted with ethyl acetate (20 mL) for three times. The combined organic layers were washed with brine (20 mL), dried over Na₂SO₄₍ₛ₎, and filtered. The filtrate was concentratede and purified by C18 column (acetonitrile:water=5% to 100%) to give the title compound (540 mg, 99.7% purity from LCMS, 87% yield) as a white solid. LC-MS (ESI): R_{T}=1.18 min, mass calcd. for C₂₆H₂₂Cl₂F₃N₅O₄ 595.1 m/z found 595.9 [M+H]⁺.

Compound 9

(3R,7S)-2-(3,4-Dichlorobenzoyl)-N,3-dimethyl-10-oxo-9-(1-(2-(trifluoromethyl)pyridin-4-yl)ethyl)-1,2,3,4,7,8,9,10-octahydropyrido[4′,3′:3,4]pyrazolo[1,5-a]pyrazine-7-carboxamide

To a solution of (3R,7S)-2-(3,4-dichlorobenzoyl)-3-methyl-10-oxo-9-(1-(2-(trifluoromethyl)pyridin-4-yl)ethyl)-1,2,3,4,7,8,9,10-octahydropyrido[4′,3′:3,4]pyrazolo[1,5-a]pyrazine-7-carboxylic acid 9-6 (540 mg, 99.7% purity, 0.90 mmol), methanamine hydrochloride (185 mg, 2.74 mmol), N1-((ethylimino)methylene)-N3,N3-dimethylpropane-1,3-diamine hydrochloride (347 mg, 1.81 mmol) and 1-hydroxybenzotriazole (245 mg, 1.81 mmol) in N,N-dimethylformamide (12 mL) was added triethylamine (1.3 ml, 9.35 mmol) at 0° C. The reaction mixture was allowed to slowly return to room temperature. After being stirred at room temperature under nitrogen atmosphere for overnight. The reaction mixture was quenched with water (20 mL) and extracted with ethyl acetate (20 mL) for three times. The combined organic layers were dried over Na₂SO₄₍ₛ₎ and filtered. The filtrate was concentratede and purified by C18 column (acetonitrile:water=5% to 100%) to give the title compound (150 mg, 99.2% purity from LCMS, 27% yield) as a white solid. LC-MS (ESI): R_{T}=1.297 min, mass calcd. for C₂₇H₂₅Cl₂F₃N₆O₃ 608.1 m/z found 609.0 [M+H]⁺.

Compounds 9A and 9B

(3R,7S)-2-(3,4-Dichlorobenzoyl)-N,3-dimethyl-10-oxo-9-((R*)-1-(2-(trifluoromethyl)pyridin-4-yl)ethyl)-1,2,3,4,7,8,9,10-octahydropyrido[4′,3′:3,4]pyrazolo[1,5-a]pyrazine-7-carboxamide (9A), and (3R,7S)-2-(3,4-Dichlorobenzoyl)-N,3-dimethyl-10-oxo-9-((S*)-1-(2-(trifluoromethyl)pyridin-4-yl)ethyl)-1,2,3,4,7,8,9,10-octahydropyrido[4′,3′:3,4]pyrazolo[1,5-a]pyrazine-7-carboxamide (9B)

A racemic mixture of (3R,7S)-2-(3,4-dichlorobenzoyl)-N,3-dimethyl-10-oxo-9-(1-(2-(trifluoromethyl)pyridin-4-yl)ethyl)-1,2,3,4,7,8,9,10-octahydropyrido[4′,3′:3,4]pyrazolo[1,5-a]pyrazine-7-carboxamide compound 9 (300 mg, 98.9% purity, 0.49 mmol) was separated by chiral prep. HPLC (Column: Chiralpak IE, 5 μm, 20*250 mm; Mobile Phase: Hex:EtOH=40:60 at 30 g/min; Temp: 30° C.; Wavelength: 254 nm) to afford compound 9A (89 mg, 29% yield, 98.3% purity from LCMS, 100% stereopure) as a white solid and compound 9B (110 mg, 37% yield, 99.8% purity from LCMS, 99.9% stereopure) as a white solid.

Compound 9A

LC-MS (ESI): R_{T}=3.982 min, mass calcd. for C₂₇H₂₅Cl₂F₃N₆O₃ 608.1 m/z found 609.1 [M+H]⁺. Chiral analysis (Column: Chiralpak IE 5 μm 4.6*250 mm; Mobile Phase: Hex:EtOH=40:60 at 1.0 mL/min; Temp: 30° C.; Wavelength: 254 nm; R_{T}=6.721 min). ¹H NMR (400 MHz, DMSO-d₆) δ 8.78-8.70 (m, 1H), 7.85-7.61 (m, 4H), 7.53 (br, s, 1H), 7.46 (d, J=8 Hz, 1H), 5.86-5.72 (m, 1H), 5.44-5.24 (m, 1H), 5.07 (s, 1H), 4.65-4.41 (m, 1H), 4.24-4.02 (m, 2H), 3.51-3.41 (m, 1H), 2.96-2.85 (m, 1H), 2.60-2.52 (m, 1H), 2.40 (d, J=4.4 Hz, 3H), 1.64-1.47 (m, 3H), 1.30-1.12 (m, 3H). ¹⁹F NMR (376 MHz, DMSO-d₆) δ −66.28.

Compound 9B

LC-MS (ESI): R_{T}=4.013 min, mass calcd. for C₂₇H₂₅Cl₂F₃N₆O₃ 608.1 m/z found 609.1 [M+H]⁺. Chiral analysis (Column: Chiralpak IE 5 μm 4.6*250 mm; Mobile Phase: Hex:EtOH=40:60 at 1.0 mL/min; Temp: 30° C.; Wavelength: 254 nm; R_{T}=7.922 min). ¹H NMR (400 MHz, DMSO-d₆) δ 8.73 (s, 1H), 8.10-7.99 (m, 1H), 7.85-7.70 (m, 3H), 7.59 (br s, 1H), 7.44 (d, J=7.6 Hz, 1H), 5.81-5.56 (m, 1H), 5.50-5.16 (m, 1H), 5.07 (s, 1H), 4.67-4.39 (m, 1H), 4.27-4.08 (m, 1H), 3.90-3.66 (m, 2H), 2.97-2.84 (m, 1H), 2.67-2.67 (m, 3H), 2.65-2.60 (m, 1H), 1.62-1.44 (m, 3H), 1.28-1.11 (m, 3H). ¹⁹F NMR (376 MHz, DMSO-d₆) δ −66.29.

Compounds 10A and 10B

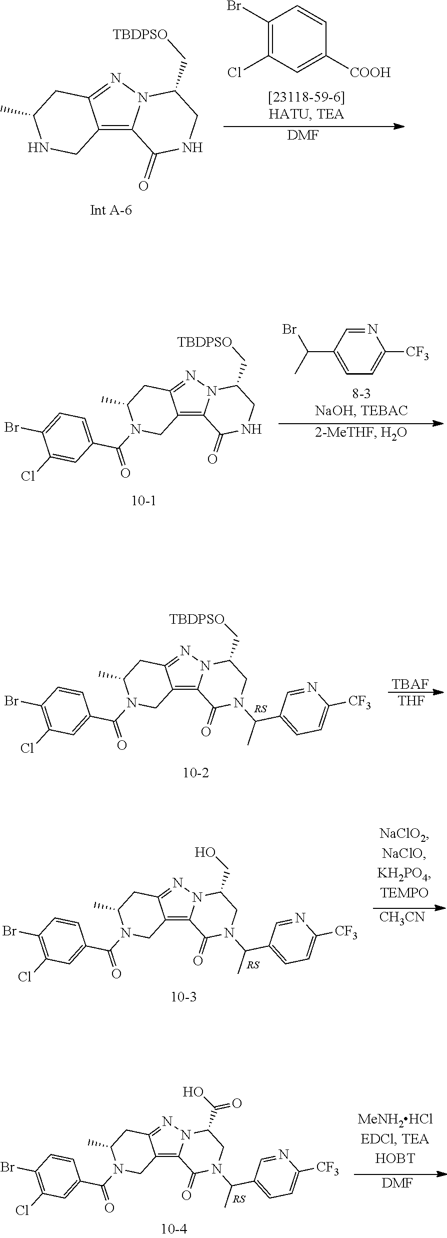

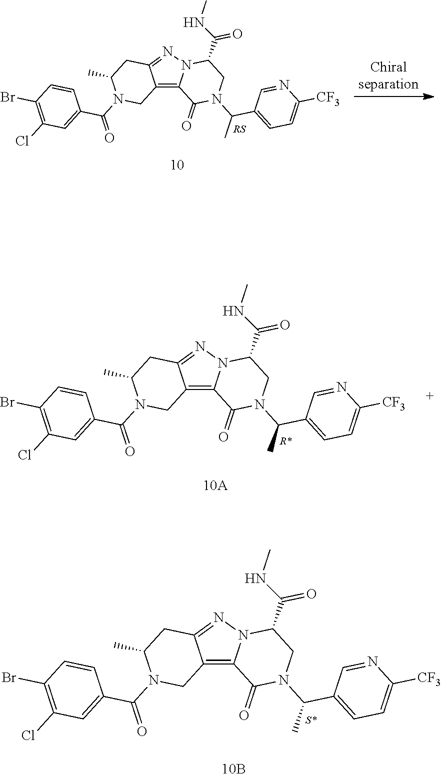 [SMILES: CNC(=O)[C@@H]1CN(C(C)c2ccc(C(F)(F)F)nc2)C(=O)c2c3c(nn21)C[C@@H](C)N(C(=O)c1ccc(Br)c(Cl)c1)C3 ; CNC(=O)[C@@H]1CN([C@H](C)c2ccc(C(F)(F)F)nc2)C(=O)c2c3c(nn21)C[C@@H](C)N(C(=O)c1ccc(Br)c(Cl)c1)C3 ; CNC(=O)[C@@H]1CN([C@@H](C)c2ccc(C(F)(F)F)nc2)C(=O)c2c3c(nn21)C[C@@H](C)N(C(=O)c1ccc(Br)c(Cl)c1)C3]

Intermediate 10-1

(3R,7S)-2-(4-Bromo-3-chlorobenzoyl)-7-(((tert-butyldiphenylsilyl)oxy)methyl)-3-methyl-1,2,3,4,8,9-hexahydropyrido[4′,3′:3,4]pyrazolo[1,5-a]pyrazin-10(7H)-one

To a mixture of (3R,7S)-7-(((tert-butyldiphenylsilyl)oxy)methyl)-3-methyl-1,2,3,4,8,9-hexahydropyrido[4′,3′:3,4]pyrazolo[1,5-a]pyrazin-10(7H)-one Int A-6 (1.5 g, 2.53 mmol, 80% purity), 4-bromo-3-chlorobenzoic acid (0.6 g, 2.55 mmol) and O-(7-azabenzotriazol-1-yl)-N,N,N′,N′-tetramethyluronium hexafluorophosphate (1.45 g, 3.81 mmol) in N,N-dimethylformide (30 mL) was added triethylamine (0.8 g, 7.91 mmol) at room temperature. The mixture was stirred at 20° C. for 12 hours. The reaction mixture was added water (80 mL) and extracted with dichloromethane (80 mL) for three times. The combined organic layers were washed with brine (80 mL) and concentrated in vacuum. The residue was purified by column chromatography on silica gel (petroleum ether:acetone=10:1) to give desired compound (1.6 g, 82% yield, 90% purity from ¹H NMR) as a white solid. ¹H NMR (300 MHz, CDCl₃) δ 7.67-7.62 (m, 5H), 7.60-7.37 (m, 7H), 7.26-7.25 (m, 1H), 7.15-7.15 (m, 1H), 5.68-5.47 (m, 1H), 4.81-4.19 (m, 3H), 4.06-4.01 (m, 1H), 3.94-3.75 (m, 3H), 3.12-2.89 (m, 1H), 2.67-2.57 (m, 1H), 1.26-1.12 (m, 3H), 1.05 (s, 9H).

Intermediate 10-2

(3R,7S)-2-(4-Bromo-3-chlorobenzoyl)-7-(((tert-butyldiphenylsilyl)oxy)methyl)-3-methyl-9-(1-(6-(trifluoromethyl)pyridin-3-yl)ethyl)-1,2,3,4,8,9-hexahydropyrido[4′,3′:3,4]pyrazolo[1,5-a]pyrazin-10(7H)-one

To a solution of (3R,7S)-2-(4-bromo-3-chlorobenzoyl)-7-(((tert-butyldiphenylsilyl)oxy)methyl)-3-methyl-1,2,3,4,8,9-hexahydropyrido[4′,3′:3,4]pyrazolo[1,5-a]pyrazin-10(7H)-one 10-1 (700 mg, 90% purity, 0.91 mmol), 5-(1-bromoethyl)-2-(trifluoromethyl)pyridine 8-3 (390 mg, 90% purity, 1.38 mmol) and N-benzyl-N,N-diethylethanaminium chloride (30 mg, 0.13 mmol) in 2-methyltetrahydrofuran (7 mL) and 50% wt. Sodium hydroxide solution (3.5 mL) at 0° C. Then the reaction mixture was stirred at room temperature overnight. The reaction mixture was quenched with water (30 mL) and extracted with ethyl acetate (30 mL) twice. The combined organic layers were dried over Na₂SO₄₍ₛ₎ and filtered. The filtrate was concentrated under reduced pressure to give crude, which was purified by C18 column (acetonitrile:water=5% to 90%) to give the title compound (600 mg, 100% purity from LCMS, 76% yield) as a colorless oil. LC-MS (ESI): R_{T}=1.34 min, mass calcd. for C₄₂H₄₂BrClF₃N₅O₃Si 863.2 m/z found 864.2 [M+H]⁺.

Intermediate 10-3

(3R,7S)-2-(4-Bromo-3-chlorobenzoyl)-7-(hydroxymethyl)-3-methyl-9-(1-(6-(trifluoromethyl)pyridin-3-yl)ethyl)-1,2,3,4,8,9-hexahydropyrido[4′,3′:3,4]pyrazolo[1,5-a]pyrazin-10(7H)-one

To a solution of (3R,7S)-2-(4-bromo-3-chlorobenzoyl)-7-(((tert-butyldiphenylsilyl)oxy)methyl)-3-methyl-9-(1-(6-(trifluoromethyl)pyridin-3-yl)ethyl)-1,2,3,4,8,9-hexahydropyrido[4′,3′:3,4]pyrazolo[1,5-a]pyrazin-10(7H)-one 10-2 (600 mg, 100% purity, 0.69 mmol) in tetrahydrofuran (6 mL) was added 1 M tetrabutylammonium fluoride in tetrahydrofuran (1.5 mL, 1.5 mmol) at room temperature, then the mixture was stirred at room temperature for 3 hours. The reaction mixture was poured into water (20 mL) and extracted with ethyl acetate (30 mL) for three times. The combined organic layers were dried over Na₂SO₄₍ₛ₎ and filtered. The filtrate was concentrated under reduced pressure to give the residue, which was purified by column gel column chromatography (dichloromethane methanol=40:1) to give the title compound (410 mg, 97% purity from LCMS, 91% yield) as a white solid. LC-MS (ESI): R_{T}=1.426 min, mass calcd. for C₂₆H₂₄BrClF₃N₅O₃ 625.1 m/z found 627.9 [M+H]⁺.

Intermediate 10-4

(3R,7S)-2-(4-Bromo-3-chlorobenzoyl)-3-methyl-10-oxo-9-(1-(6-(trifluoromethyl)pyridin-3-yl)ethyl)-1,2,3,4,7,8,9,10-octahydropyrido[4′,3′:3,4]pyrazolo[1,5-a]pyrazine-7-carboxylic acid

To a solution of (3R,7S)-2-(4-bromo-3-chlorobenzoyl)-7-(hydroxymethyl)-3-methyl-9-(1-(6-(trifluoromethyl)pyridin-3-yl)ethyl)-1,2,3,4,8,9-hexahydropyrido[4′,3′:3,4]pyrazolo[1,5-a]pyrazin-10(7H)-one 10-3 (410 mg, 97% purity, 0.63 mmol) in acetonitrile (4 mL) was added saturated aqueous potassium dihydrogen phosphate solution (6 mL), 2,2,6,6-tetramethylpiperidinooxy (200 mg, 1.28 mmol), sodium chlorite (145 mg, purity 80%, 1.28 mmol), 5.5% sodium hypochlorite solution (0.8 mL, 1.34 mmol) at 0° C. The reaction mixture was allowed to slowly return to room temperature. After being stirred at room temperature for 5 hours, the reaction mixture was quenched with saturated sodium thiosulfate aqueous solution (5 mL), acidified with 1 M hydrochloric acid solution to pH=4˜5, and extracted with ethyl acetate (20 mL) for three times. The combined organic layers were washed with brine (20 mL), dried over Na₂SO₄₍ₛ₎, and filtered. The filtrate was concentrated and purified by C18 column (acetonitrile:water=5% to 100%) to give the title compound (300 mg, 97% purity from LCMS, 72% yield) as a white solid. LC-MS (ESI): R_{T}=1.060 min, mass calcd. for C₂₆H₂₂BrClF₃N₅O₄ 639.1 m/z found 639.9 [M+H]⁺.

Compound 10

(3R,7S)-2-(4-Bromo-3-chlorobenzoyl)-N,3-dimethyl-10-oxo-9-(1-(6-(trifluoromethyl)pyridin-3-yl)ethyl)-1,2,3,4,7,8,9,10-octahydropyrido[4′,3′:3,4]pyrazolo[1,5-a]pyrazine-7-carboxamide

To a solution of (3R,7S)-2-(4-bromo-3-chlorobenzoyl)-3-methyl-10-oxo-9-(1-(6-(trifluoromethyl)pyridin-3-yl)ethyl)-1,2,3,4,7,8,9,10-octahydropyrido[4′,3′:3,4]pyrazolo[1,5-a]pyrazine-7-carboxylic acid 10-4 (270 mg, 97% purity, 0.41 mmol), methanamine hydrochloride (83 mg, 1.23 mmol), N1-((ethylimino)methylene)-N3,N3-dimethylpropane-1,3-diamine hydrochloride (157 mg, 0.82 mmol) and 1-hydroxybenzotriazole (111 mg, 0.82 mmol) in N,N-dimethylformamide (6 mL) was added triethylamine (0.6 ml, 4.32 mmol) at 0° C. The reaction mixture was allowed to slowly return to room temperature. After being stirred at room temperature under nitrogen atmosphere for overnight. The reaction mixture was quenched with water (20 mL) and extracted with ethyl acetate (20 mL) three times. The combined organic layers were dried over Na₂SO₄₍ₛ₎ and filtered. The filtrate was concentratede and purified by C18 column (acetonitrile:water=5% to 100%) to give the title compound (155 mg, 100% purity from LCMS, 58% yield) as a white solid. LC-MS (ESI): R_{T}=1.61 min, mass calcd. for C₂₇H₂₅BrClF₃N₆O₃ 652.1 m/z found 653.3 [M+H]⁺.

Compounds 10A and 10B

(3R,7S)-2-(4-bromo-3-chlorobenzoyl)-N,3-dimethyl-10-oxo-9-((R*)-1-(6-(trifluoromethyl)pyridin-3-yl)ethyl)-1,2,3,4,7,8,9,10-octahydropyrido[4′,3′:3,4]pyrazolo[1,5-a]pyrazine-7-carboxamide (10A), and (3R,7S)-2-(4-bromo-3-chlorobenzoyl)-N,3-dimethyl-10-oxo-9-((S*)-1-(6-(trifluoromethyl)pyridin-3-yl)ethyl)-1,2,3,4,7,8,9,10-octahydropyrido[4′,3′:3,4]pyrazolo[1,5-a]pyrazine-7-carboxamide (10B)

A racemic mixture of (3R,7S)-2-(4-bromo-3-chlorobenzoyl)-N,3-dimethyl-10-oxo-9-(1-(6-(trifluoromethyl)pyridin-3-yl)ethyl)-1,2,3,4,7,8,9,10-octahydropyrido[4′,3′:3,4]pyrazolo[1,5-a]pyrazine-7-carboxamide compound 10 (170 mg, 100% purity, 0.26 mmol) was separated by chiral prep. HPLC (Column: Chiralpak IE, 5 μm, 20*250 mm; Mobile Phase: ACN:IPA=70:30 at 30 g/min; Temp: 30° C.; Wavelength: 254 nm) to afford compound 10A (34 mg, 99.5% purity from LCMS, 20% yield, 100% stereopure) as a white solid and compound 10B (91 mg, 99.5% purity from LCMS, 53% yield, 99.7% stereopure) as a white solid.

Compound 10A

LC-MS (ESI): R_{T}=3.771 min, mass calcd. for C₂₇H₂₅BrClF₃N₆O₃ 652.1 m/z found 653.1 [M+H]⁺. Chiral analysis: (Column: Chiralpak IE 5 μm 4.6*250 mm; Mobile Phase: ACN:IPA=70:30 at 1.0 mL/min; Temp: 30° C.; Wavelength: 254 nm; R_{T}=4.309 min). ¹H NMR (400 MHz, DMSO-d₆) δ 8.62 (s, 1H), 7.99-7.85 (m, 3H), 7.79-7.68 (m, 2H), 7.36 (d, J=7.6 Hz, 1H), 5.98-5.76 (m, 1H), 5.48-5.19 (m, 1H), 5.01 (s, 1H), 4.67-4.43 (m, 1H), 4.25-3.38 (m, 2H), 3.48-3.38 (m, 1H), 2.94-2.85 (m, 1H), 2.62-2.52 (m, 1H), 2.36-2.30 (m, 3H), 1.64-1.48 (m, 3H), 1.27-1.12 (m, 3H). ¹⁹F NMR (376 MHz, DMSO-d₆) δ −66.38.

Compound 10B

LC-MS (ESI): R_{T}=3.855 min, mass calcd. for C₂₇H₂₅BrClF₃N₆O₃ 652.1 m/z found 653.2 [M+H]⁺. Chiral analysis: (Column: Chiralpak IE 5 μm 4.6*250 mm; Mobile Phase: ACN:IPA=70:30 at 1.0 mL/min; Temp: 30° C.; Wavelength: 254 nm; R_{T}=4.752 min). ¹H NMR (400 MHz, DMSO-d₆) δ 8.80-8.68 (m, 1H), 8.04 (s, 2H), 7.94-7.83 (m, 2H), 7.73 (s, 1H), 7.35 (d, J=8.4 Hz, 1H), 5.86-5.66 (m, 1H), 5.46-5.21 (m, 1H), 5.06 (s, 1H), 4.61-4.41 (m, 1H), 4.25-4.09 (m, 1H), 3.88-3.69 (m, 2H), 2.95-2.84 (m, 1H), 2.64 (d, J=4.4 Hz, 3H), 2.58-2.52 (m, 1H), 1.60-1.44 (m, 3H), 1.27-1.11 (m, 3H). ¹⁹F NMR (376 MHz, DMSO-d₆) δ −66.31.

Compounds 11A and 11B

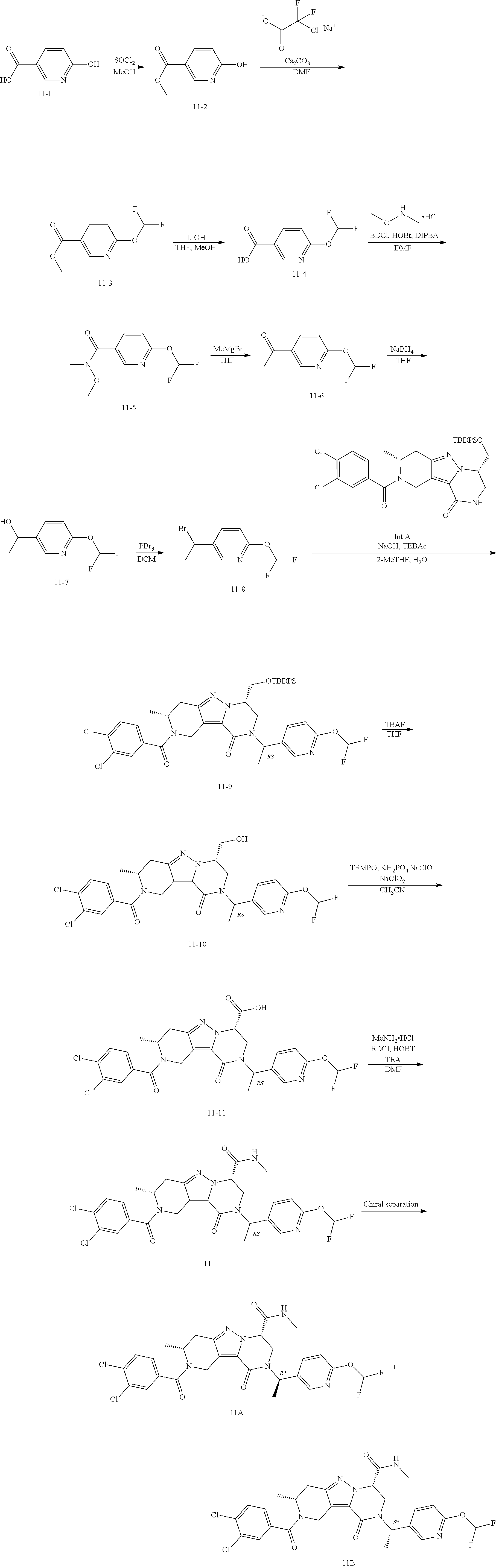

Intermediate 11-2

Methyl 6-hydroxynicotinate

To the solution of 6-hydroxynicotinic acid 11-1 (2.0 g, 14.4 mmol) in methanol (20 mL) was added sulfurous dichloride (2 mL, 27.6 mmol) at 0° C. The mixture was stirred at 75° C. for 4 hours. The mixture was concentrated to give the title compound (2.18 g, 90% purity from NMR, 89.1% yield) as yellow solids. ¹H NMR (300 MHz, DMSO-d₆) δ 10.59 (br s, 1H), 8.05 (d, J=2.1 Hz, 1H), 7.80 (dd, J=9.6, 1.8 Hz, 1H), 6.38 (d, J=9.6 Hz, 1H), 3.77 (s, 3H).

Intermediate 11-3

Methyl 6-(difluoromethoxy)nicotinate

To the solution of methyl 6-hydroxynicotinate 11-2 (1.18 g, 90% purity, 6.94 mmol) in N,N-dimethylformamide (20 mL) was added cesium carbonate (6.9 g, 21.2 mmol) and sodium 2-chloro-2,2-difluoroacetate (2.1 g, 13.8 mmol). Then the mixture was stirred at 95° C. for 4 hours. The mixture was diluted with water (100 mL) and extracted with ethyl acetate (50 mL) twice. The combined organic layers were washed with brine (50 mL), dried over Na₂SO₄₍ₛ₎ and filtered. The filtrate was concentrated and purified by silica gel column chromatography (petroleum ether:ethyl acetate=4:1) to give the title compound (500 mg, 95% purity from NMR, 33.7% yield) as white solids. ¹H NMR (400 MHz, CDCl₃) δ 8.84 (d, J=2.0 Hz, 1H), 8.32 (dd, J=8.4 and 2.4 Hz, 1H), 7.54 (t, J=72.4 Hz, 1H), 6.95 (d, J=9.2 Hz, 1H), 3.94 (s, 3H).

Intermediate 11-4

6-(Difluoromethoxy)nicotinic acid

To a solution of methyl 6-(difluoromethoxy)nicotinate 11-3 (1.2 g, 95% purity, 5.61 mmol) in tetrahydrofuran (5 mL) and methanol (5 mL) was added a solution of lithium hydroxide monohydrate (750 mg, 17.9 mmol) in water (5 mL) at 0° C. After being stirred 0° C. for 1 hour, the mixture was acidified to pH=6 with 0.1 M hydrochloride aqueous solution and extracted with ethyl acetate (30 mL) twice. The combined organic layers were washed with water (30 mL) for three times and brine (30 mL), dried over Na₂SO₄₍ₛ₎ and filtered. The filtrate was concentrated under reduced pressure to give the title compound (1.05 g, 100% purity from LCMS, 98.9% yield) as yellow solids. LC-MS (ESI): R_{T}=0.694 min, mass calcd. for C₇H₅F₂NO₃ 189.1, m/z found 188.1 [M−H]⁻.

Intermediate 11-5

6-(Difluoromethoxy)-N-methoxy-N-methylnicotinamide

A mixture of 6-(difluoromethoxy)nicotinic acid 11-4 (1.05 g, 100% purity, 5.55 mmol), N-ethyl-N-isopropylpropan-2-amine (7.4 mL, 44.8 mmol), N,0-dimethylhydroxylamine hydrochloride (1.17 g, 12.0 mmol), 1-ethyl-(3-(3-dimethylamino)propyl)-carbodiimide hydrochloride (1.7 g, 8.87 mmol) and benzotriazol-1-ol (1.17 g, 8.66 mmol) in N,N-dimethylformamide (10 mL) was stirred at 30° C. under nitrogen for 14 hours. The mixture was acidified to pH=6 with 0.5 M hydrochloride aqueous solution and extracted with ethyl acetate (50 mL) twice. The combined organic layers were washed with water (40 mL) for three times and brine (40 mL), dried over Na₂SO₄₍ₛ₎ and filtered. The filtrate was concentrated under reduced pressure to give a residue, which was purified by C18 column (acetonitrile: water=45% to 75%) to give the title compound (1.12 g, 90% purity from NMR, 78.2% yield) as a yellow oil LC-MS (ESI): R_{T}=1.46 min, mass calcd. for C₉H₁₀F₂N₂O₃ 232.1, m/z found 233.0 [M+H]⁺. ¹H NMR (400 MHz, CDCl₃) δ 8.63 (d, J=2.0 Hz, 1H), 8.13 (dd, J=8.8 and 2.4 Hz, 1H), 7.52 (t, J=72.8 Hz, 1H), 6.93 (d, J=8.8 Hz, 1H), 3.57 (s, 3H), 3.39 (s, 3H).

Intermediate 11-6

1-(6-(Difluoromethoxy)pyridin-3-yl)ethanone

To a solution of 6-(difluoromethoxy)-N-methoxy-N-methylnicotinamide 11-5 (1.12 g, 90% purity, 4.34 mmol) in tetrahydrofuran (5 mL) was added dropwise 3 M methylmagnesium bromide in tetrahydrofuran (3 mL, 9.0 mmol) at 0° C. After being stirred at room temperature for 2 hours, the reaction mixture was poured into saturated aqueous ammonium chloride solution (30 mL) and extracted with ethyl acetate (40 mL) for twice. The combined organic layers were washed with brine (30 mL), dried over Na₂SO₄₍ₛ₎ and filtered. The filtrate was concentrated to give the title compound (900 mg, 90% purity from NMR, 99.7% yield) as a yellow oil. LC-MS (ESI): R_{T}=1.50 min, mass calcd. for C₈H₇F₂NO₂ 187.1, m/z found 188.1 [M+H]⁺. ¹H NMR (400 MHz, CDCl₃) δ 8.78 (d, J=2.4 Hz, 1H), 8.30 (dd, J=8.4 and 2.4 Hz, 1H), 7.54 (t, J=72.0 Hz, 1H), 6.98 (d, J=8.8 Hz, 1H), 2.61 (s, 3H).

Intermediate 11-7

1-(6-(Difluoromethoxy)-3-yl)ethanol

To a solution of 1-(6-(difluoromethoxy)-3-yl)ethanone 11-6 (900 mg, 90% purity, 4.33 mmol) in tetrahydrofuran (5 mL) was added sodium borohydride (500 mg, 13.2 mmol) at 0° C. After addition, the mixture was stirred at room temperature for 2 hours. The mixture was quenched with water (30 mL). The organic phase was separated, and the aqueous phase was extracted with ethyl acetate (30 mL) for three times. The combined organic layers were washed with brine (20 mL), dried over Na₂SO₄₍ₛ₎, and filtered and concentrated under reduced pressure to get the desired product (715 mg, 90% purity, 78.6% yield) as a yellow oil. LC-MS (ESI): R_{T}=1.39 min, mass calcd. for C₈H₉F₂NO₂ 189.1, m/z found 190.0 [M+H]⁺. ¹H NMR (400 MHz, CDCl₃) δ 8.16 (d, J=2.4 Hz, 1H), 7.78 (dd, J=8.4, 2.0 Hz, 1H), 7.45 (t, J=73.2 Hz, 1H), 6.89 (d, J=8.8 Hz, 1H), 4.94 (q, J=6.4 Hz, 1H), 1.51 (d, J=6.4 Hz, 3H).

Intermediate 11-8

5-(1-Bromoethyl)-2-(difluoromethoxy)pyridine

To a solution of 1-(6-(difluoromethoxy)-3-yl)ethanol 11-7 (715 mg, 90% purity, 3.4 mmol) in dichloromethane (5 mL) was added tribromophosphine (0.2 mL, 2.13 mmol) at 0° C. After being stirred at 0° C. for 0.5 hour, the mixture was quenched by the addition of water. The organic phase was washed with saturated aqueous sodium bicarbonate solution, dried over MgSO₄ and filtered. The filtrate was concentrated under reduced pressure to give the crude compound, which was purified by silica gel column chromatography (petroleum ether:ethyl acetate=100:1) to give the title compound (260 mg, 90% purity from ¹H NMR, 27.3% yield) as a yellow oil. LC-MS (ESI): R_{T}=1.72 min, mass calcd. for C₈H₈BrF₂NO 251.0, m/z found 252.1 [M+H]⁺. ¹H NMR (400 MHz, CDCl₃) δ 8.21 (d, J=2.8 Hz, 1H), 7.85 (dd, J=8.8, 2.8 Hz, 1H), 7.46 (t, J=72.8 Hz, 1H), 6.91 (d, J=8.4 Hz, 1H), 5.18 (q, J=6.8 Hz, 1H), 2.04 (d, J=7.2 Hz, 3H).

Intermediate 11-9

(3R,7S)-7-(((tert-Butyldiphenylsilyl)oxy)methyl)-2-(3,4-dichlorobenzoyl)-9-(1-(6-(difluoromethoxy)pyridin-3-yl)ethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (600 mg, 100% purity, 0.926 mmol) in 2-methyltetrahydrofuran (6 mL) and 50% wt. sodium hydroxide in water (6 mL, 333 mmol) was added 5-(1-bromoethyl)-2-(difluoromethoxy)pyridine 11-8 (525 mg, 90% purity, 1.87 mmol) and benzyltriethylammonium chloride (30 mg, 0.132 mmol) at room temperature. After being stirred at room temperature for 2 hours, the reaction mixture was quenched with water (10 mL) and extracted with ethyl acetate (10 mL) twice. The combined organic layers were washed with brine (10 mL), dried over Na₂SO₄₍ₛ₎ and filtered. The filtrate was concentrated to give the title compound (800 mg, 73% purity, 77% yield) as white solids. LC-MS (ESI): R_{T}=2.20 min, mass calcd. for C₄₂H₄₃Cl₂F₂N₅O₄Si 817.2, m/z found 818.5 [M+H]⁺.

Intermediate 11-10

(3R,7S)-2-(3,4-Dichlorobenzoyl)-9-(1-(6-(difluoromethoxy)pyridin-3-yl)ethyl)-7-(hydroxymethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-9-(1-(6-(difluoromethoxy)pyridin-3-yl)ethyl)-3-methyl-1,2,3,4,8,9-hexahydropyrido[4′,3′:3,4]pyrazolo[1,5-a]pyrazin-10(7H)-one 11-9 (800 mg, 73% purity, 0.713 mmol) in tetrahydrofuran (10 mL) was added 1 M tetrabutylammonium fluoride in tetrahydrofuran (1.2 mL, 1.2 mmol) at room temperature. After being stirred at room temperature for 1 hour, the reaction mixture was quenched with water (10 mL) and extracted with ethyl acetate (10 mL) twice. The combined organic layers were washed with brine (20 mL) twice, dried over Na₂SO₄₍ₛ₎ and filtered. The filtrate was concentrated and purified by column chromatography on silica gel (dichloromethane:ethyl acetate=10:1) to give the title compound (380 mg, 90% purity from ¹H NMR, 82.6% yield) as white solids. ¹H NMR (400 MHz, CDCl₃) δ 8.27-8.23 (m, 1H), 7.81-7.71 (m, 1H), 7.60-7.49 (m, 3H), 6.94 (d, J=8.4 Hz, 1H), 6.20-5.96 (m, 1H), 5.26-4.85 (m, 1H), 4.56-4.13 (m, 3H), 4.09-3.79 (m, 2H), 3.74-3.53 (m, 1H), 3.37-3.02 (m, 2H). 2.76-2.62 (m, 1H), 2.21 (s, 2H), 1.74-1.64 (m, 3H), 1.40-1.12 (m, 3H). LC-MS (ESI): R_{T}=1.56 min, mass calcd. for C₂₆H₂₅Cl₂F₂N₅O₄ 579.1, m/z found 580.0 [M+H]⁺.

Intermediate 11-11

(3R,7S)-2-(3,4-Dichlorobenzoyl)-9-(1-(6-(difluoromethoxy)pyridin-3-yl)ethyl)-3-methyl-10-oxo-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-9-(1-(6-(difluoromethoxy)pyridin-3-yl)ethyl)-7-(hydroxymethyl)-3-methyl-1,2,3,4,8,9-hexahydropyrido[4′,3′:3,4]pyrazolo[1,5-a]pyrazin-10(7H)-one 11-10 (380 mg, 90% purity, 0.589 mmol), sodium chlorite (90 mg, 1.21 mmol) and 2,2,6,6-tetramethylpiperidinooxy (185 mg, 1.18 mmol) in acetonitrile (5 mL) was added saturated aqueous potassium phosphate monobasic solution (5 mL) and 10% sodium hypochlorite aqueous solution (0.9 mL, 1.24 mmol) at 0° C. After being stirred at room temperature overnight, the reaction mixture was quenched with saturated sodium sulfite aqueous solution (10 mL), acidized with 1 M hydrochloride aqueous solution to pH˜4, and extracted with ethyl acetate (10 mL) twice. The combined organic layers were dried over Na₂SO₄₍ₛ₎, and filtered. The filtrate was concentrated, and purified by C18 column (acetonitrile:water=50% to 60%) to give the title compound (300 mg, 100% purity from LCMS, 85.7% yield) as white solids. LC-MS (ESI): R_{T}=1.29 min, mass calcd. for C₂₆H₂₃Cl₂F₂N₅O₅ 593.1, m/z found 594.0[M+H]⁺.

Compound 11

(3R,7S)-2-(3,4-Dichlorobenzoyl)-9-(1-(6-(difluoromethoxy)pyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide

To a solution of (3R,7S)-2-(3,4-dichlorobenzoyl)-9-(1-(6-(difluoromethoxy)pyridin-3-yl)ethyl)-3-methyl-10-oxo-1,2,3,4,7,8,9,10-octahydropyrido[4′,3′:3,4]pyrazolo[1,5-a]pyrazine-7-carboxylic acid 11-11 (220 mg 100%, purity, 0.37 mmol), 1-(3-Dimethylaminopropyl)-3-ethylcarbodiimide hydrochloride (143 mg, 0.746 mmol), methanamine hydrochloride (55 mg, 0.815 mmol) and 1H-benzo[d][1,2,3]triazol-1-ol (99 mg, 0.733 mmol) in N,N-dimethylformamide (5 mL) was added triethylamine (0.4 mL, 2.26 mmol) at 0° C. After being stirred at room temperature for 3 hours, the mixture was quenched with saturated ammonium chloride aqueous solution (10 mL) and extracted with ethyl acetate (10 mL) twice. The combined organic layers were washed with brine (20 mL), dried over Na₂SO₄₍ₛ₎ and filtered. The filtrate was concentrated under reduced pressure to give a residue, which was purified by C18 column (acetonitrile:water (0.1% ammonium bicarbonate=45% to 55%) to give the title compound (200 mg, 100% purity, 88.9% yield) as yellow solids. LC-MS (ESI): R_{T}=1.54 min, mass calcd. for C₂₇H₂₆Cl₂F₂N₆O₄ 606.1, m/z found 607.4 [M+H]⁺.

Compounds 1A and 11B

(3R,7S)-2-(3,4-Dichlorobenzoyl)-9-((R*)-1-(6-(difluoromethoxy)pyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide (11A), and (3R,7S)-2-(3,4-dichlorobenzoyl)-9-((S*)-1-(6-(difluoromethoxy)pyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide (11B)

To a solution of (3R,7S)-2-(3,4-dichlorobenzoyl)-9-(1-(6-(difluoromethoxy)pyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide compound 11 (200 mg, 100% purity, 0.329 mmol) was separated by chiral prep. HPLC (Column: Chiralpak IE 5 μm 30*250 mm; Mobile Phase: Hex:EtOH=30:70 at 9 mL/min; Col. Temp: 30° C.; Wavelength: 254 nm) to give the compound 11A (30.3 mg, 99.4% purity, 15.1% yield, 100% stereopure) as white solids and compound 11B (72.9 mg, 98.9% purity, 36.0% yield, 99.6% stereopure) as white solids.

Compound 11A

LC-MS (ESI): R_{T}=3.594 min, mass calcd. for C₂₇H₂₆Cl₂F₂N₆O₄ 606.1, m/z found 607.1 [M+H]⁺. Chiral analysis (Column: Chiralpak IE 5 μm 4.6*250 mm; Mobile Phase: Hex:EtOH=30:70 at 1 mL/min; Temp: 30° C.; Wavelength: 254 nm; R_{T}=6.286 min). ¹H NMR (400 MHz, CDCl₃) δ 8.15 (s, 1H), 7.67-7.64 (m, 1H), 7.53-7.46 (m, 3H), 7.30-7.27 (m, 1H), 7.25-7.21 (m, 1H), 6.88 (d J=8.8 Hz, 1H), 6.01-5.36 (m, 3H), 4.90-4.83 (m, 1H), 4.58-4.33 (m, 1H), 3.97-3.83 (m, 2H), 3.19-2.98 (m, 1H), 2.75-2.64 (m, 4H), 1.68-1.60 (m, 3H), 1.33-1.26 (m, 3H). ¹⁹F NMR (376 MHz, CDCl₃) δ −88.37-−89.62.

Compound 11B

LC-MS (ESI): R_{T}=3.401 min, mass calcd. for C₂₇H₂₆Cl₂F₂N₆O₄ 606.1, m/z found 607.1 [M+H]⁺. Chiral analysis (Column: Chiralpak IE 5 μm 4.6*250 mm; Mobile Phase: Hex:EtOH=30:70 at 1 mL/min; Temp: 30° C.; Wavelength: 254 nm; R_{T}=8.462 min). ¹H NMR (400 MHz, CDCl₃) δ 8.24-8.19 (m, 1H), 7.77-7.71 (m, 1H), 7.63-7.45 (m, 3H), 7.30-7.27 (m, 1H), 7.26-7.24 (m, 1H), 6.89 (d J=8.8 Hz, 1H), 6.14-5.55 (m, 3H), 4.88-4.82 (m, 1H), 4.61-4.42 (m, 1H), 4.19-4.09 (m, 1H), 3.47-3.39 (m, 1H), 3.12-2.96 (m, 1H), 2.80 (d, J=5.2 Hz, 3H), 2.73-2.69 (m, 1H), 1.62-1.60 (m, 3H), 1.34-1.24 (m, 3H). ¹⁹F NMR (376 MHz, CDCl₃) δ −88.98-−89.18.

Compounds 12A and 12B

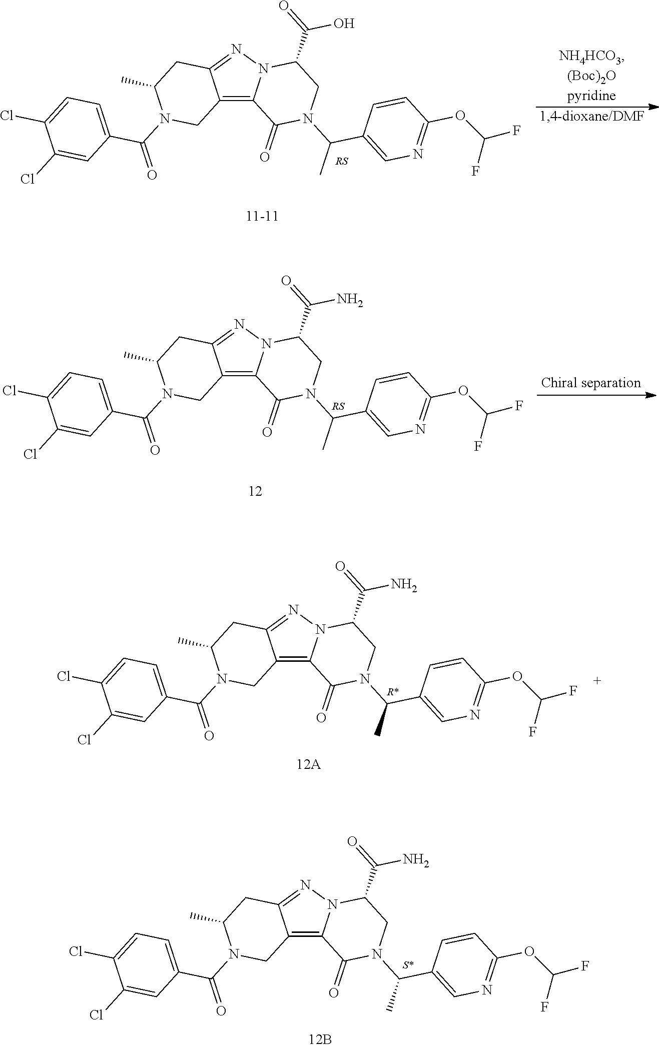

Compound 12

(3R,7S)-2-(3,4-Dichlorobenzoyl)-9-(1-(6-(difluoromethoxy)pyridin-3-yl)ethyl)-3-methyl-10-oxo-1,2,3,4,7,8,9,10-octahydropyrido[4′,3′:3,4]pyrazolo[1,5-a]pyrazine-7-carboxamide

To a solution of (3R,7S)-2-(4-chloro-3-methylbenzoyl)-3-methyl-10-oxo-9-(1-(6-(trifluoromethyl)pyridin-3-yl)ethyl)-1,2,3,4,7,8,9,10-octahydropyrido[4′,3′:3,4]pyrazolo[1,5-a]pyrazine-7-carboxylic acid 11-11 (400 mg, 93% purity, 0.626 mmol), ammonium bicarbonate (100 mg, 1.27 mmol) and di-tert-butyl dicarbonate (265 mg, 1.21 mmol) in 1,4-dioxane (9 mL) and N,N-dimethylformamide (3 mL) was added pyridine (100 mg, 1.26 mmol) at 0° C. After being stirred at 30° C. under nitrogen atmosphere for 1.5 hours, the reaction mixture was quenched with water (10 mL) and extracted with ethyl acetate (10 mL) for three times. The combined organic layers were dried over Na₂SO₄₍ₛ₎ and filtered. The filtrate was concentrated and purified by C18 column (acetonitrile:water=5% to 100%) to give the title compound (340 mg, 100% purity from LCMS, 92% yield) as yellow solids. LC-MS (ESI): R_{T}=1.57 min, mass calcd. for C₂₆H₂₄Cl₂F₂N₆O₄ 592.1, m/z found 593.1 [M+H]⁺.

Compounds 12A and 12B

(3R,7S)-2-(3,4-Dichlorobenzoyl)-9-((R*)-1-(6-(difluoromethoxy)pyridin-3-yl)ethyl)-3-methyl-10-oxo-1,2,3,4,7,8,9,10-octahydropyrido[4′,3′:3,4]pyrazolo[1,5-a]pyrazine-7-carboxamide (12A), and (3R,7S)-2-(3,4-dichlorobenzoyl)-9-((S*)-1-(6-(difluoromethoxy)pyridin-3-yl)ethyl)-3-methyl-10-oxo-1,2,3,4,7,8,9,10-octahydropyrido[4′,3′:3,4]pyrazolo[1,5-a]pyrazine-7-carboxamide (12B)

A racemic mixture of (3R,7S)-2-(3,4-dichlorobenzoyl)-9-(1-(6-(difluoromethoxy)pyridin-3-yl)ethyl)-3-methyl-10-oxo-1,2,3,4,7,8,9,10-octahydropyrido[4′,3′:3,4]pyrazolo[1,5-a]pyrazine-7-carboxamide compound 12 (340 mg, 100% purity, 0.573 mmol) was separated by chiral Prep. HPLC (separation condition: Column: Chiralpak IC 5 μm 30*250 mm; Mobile Phase: ACN=100 at 25 mL/min; Temp: 30° C.; Wavelength: 254 nm) to give compound 12A (44.4 mg, 98.2% purity from LCMS, 13% yield, 99.2% stereopure) and compound 12B (90.8 mg, 98.8% purity from LCMS, 26% yield, 100% stereopure) as white solids.

Compound 12A

LC-MS (ESI): R_{T}=3.517 min, mass calcd. for C₂₆H₂₄Cl₂F₂N₆O₄ 592.1, m/z found 593.1 [M+H]⁺. Chiral analysis (Chiralpak IC 5 um 4.6*250 mm; Mobile Phase: ACN=100 at 1 mL/min; Temp: 30° C.; Wavelength: 254 nm; Rt=5.839 min). ¹H NMR (400 MHz, CDCl₃) δ 8.17 (s, 1H), 7.70-7.69 (m, 1H), 7.64-7.46 (m, 3H), 7.28-7.25 (m, 1H), 6.87 (d, J=8.4 Hz, 1H), 6.07-5.90 (m, 2H), 5.68-5.33 (m, 2H), 4.91-4.89 (m, 1H), 4.62-4.22 (m, 2H), 3.97-3.85 (m, 2H), 3.15-2.97 (m, 1H), 2.69 (d, J=16.4 Hz, 1H), 1.63-1.61 (m, 3H), 1.29-1.28 (m, 3H).

Compound 12B

LC-MS (ESI): R_{T}=3.750 min, mass calcd. for C₂₆H₂₄Cl₂F₂N₆O₄ 592.1, m/z found 593.2 [M+H]⁺. Chiral analysis (Chiralpak IC 5 um 4.6*250 mm; Mobile Phase: ACN=100 at 1 mL/min; Temp: 30° C.; Wavelength: 254 nm; Rt=8.807 min). ¹H NMR (400 MHz, CDCl₃) δ 8.22 (s, 1H), 7.74-7.72 (m, 1H), 7.54-7.45 (m, 3H), 7.28-7.25 (m, 1H), 6.89 (d, J=8.4 Hz, 1H), 6.22-5.93 (m, 2H), 5.76-5.28 (m, 2H), 4.89 (br s, 1H), 4.67-4.29 (m, 2H), 4.10 (d, J=13.6 Hz, 1H), 3.46-3.41 (m, 1H), 3.15-2.87 (m, 1H), 2.70 (d, J=16.0 Hz, 1H), 1.63-1.62 (m, 3H), 1.28 (d, J=6.8 Hz, 3H).

Compounds 13A and 13B (Preparation Method A)

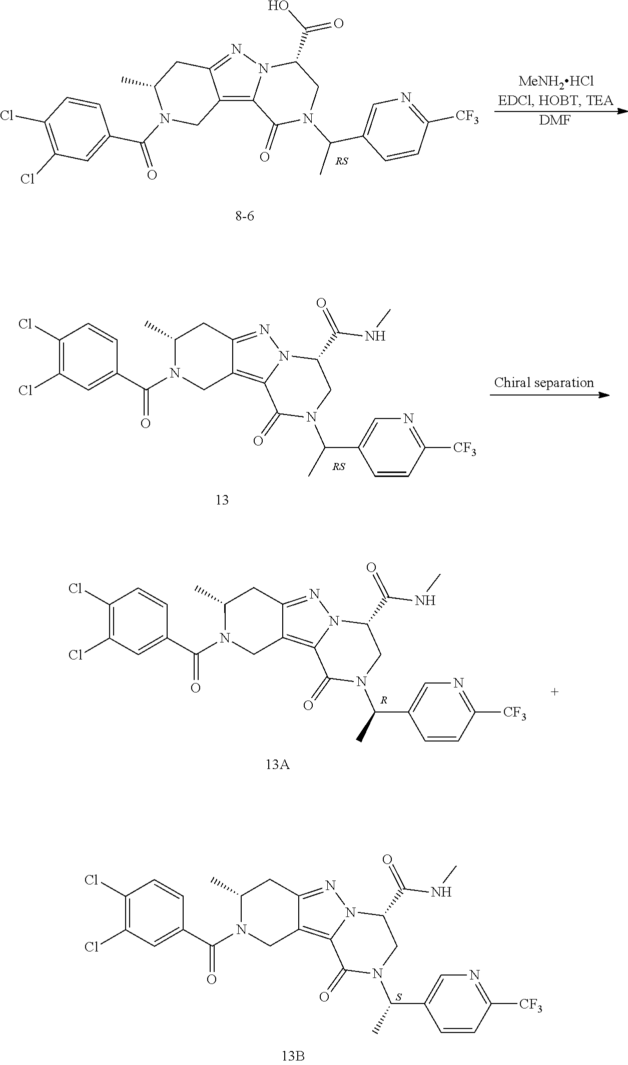

Compound 13

(3R,7S)-2-(3,4-Dichlorobenzoyl)-N,3-dimethyl-10-oxo-9-(1-(6-(trifluoromethyl)pyridin-3-yl)ethyl)-1,2,3,4,7,8,9,10-octahydropyrido[4′,3′:3,4]pyrazolo[1,5-a]pyrazine-7-carboxamide

To the solution of (3R,7S)-2-(3,4-dichlorobenzoyl)-3-methyl-10-oxo-9-(1-(6-(trifluoromethyl)pyridin-3-yl)ethyl)-1,2,3,4,7,8,9,10-octahydropyrido[4′,3′:3,4]pyrazolo[1,5-a]pyrazine-7-carboxylic acid 8-6 (330 mg, 100% purity, 0.55 mmol) in N,N-dimethylformamide (5 mL) was added methanamine hydrochloride (80 mg, 1.19 mmol), 1-hydroxybenzotriazole (150 mg, 1.11 mmol), N1-((ethylimino)methylene)-N3,N3-dimethylpropane-1,3-diamine hydrochloride (210 mg, 1.10 mmol) and triethylamine (0.6 mL, 4.31 mmol) at 0° C. The mixture was stirred at room temperature for 16 hours. The mixture was diluted with water (20 mL), acidized with 0.5 M hydrochloric acid aqueous solution to pH˜5 and extracted with ethyl acetate (50 mL) twice. The combined organic layers were washed with brine (20 mL), dried over Na₂SO₄₍ₛ₎ and filtered. The filtrate was concentrated and purified by C18 column (acetonitrile:water=5% to 100%) to give the title compound (145 mg, 100% purity from LCMS, 43.0% yield) as yellow solids. LC-MS (ESI): R_{T}=1.60 min, mass calcd. for C₂₇H₂₅Cl₂F₃N₆O₃ 608.1, m/z found 609.1 [M+H]⁺.

Compounds 13A and 13B

(3R,7S)-2-(3,4-Dichlorobenzoyl)-N,3-dimethyl-10-oxo-9-((R)-1-(6-(trifluoromethyl)pyridin-3-yl)ethyl)-1,2,3,4,7,8,9,10-octahydropyrido[4′,3′:3,4]pyrazolo[1,5-a]pyrazine-7-carboxamide (13A), and (3R,7S)-2-(3,4-dichlorobenzoyl)-N,3-dimethyl-10-oxo-9-((S)-1-(6-(trifluoromethyl)pyridin-3-yl)ethyl)-1,2,3,4,7,8,9,10-octahydropyrido[4′,3′:3,4]pyrazolo[1,5-a]pyrazine-7-carboxamide (13B)

The racemate of (3R,7S)-2-(3,4-dichlorobenzoyl)-N,3-dimethyl-10-oxo-9-(1-(6-(trifluoromethyl)pyridin-3-yl)ethyl)-1,2,3,4,7,8,9,10-octahydropyrido[4′,3′:3,4]pyrazolo[1,5-a]pyrazine-7-carboxamide compound 13 (145 mg, 100% purity, 0.24 mmol) was separated by chiral Prep. HPLC (separation condition: Column: Chiralpak IE 5 μm 30*250 mm, Mobile Phase: Hex:EtOH=30:70 at 30 mL/min; Temp: 30° C.; Wavelength: 254 nm) to afford the crude P1 and P2. The crude P1 was purified by C18 column (acetonitrile:water=5% to 100%) to give compound 13A (30 mg, 98.2% purity, 20.3% yield) as white solids and another crude peak, which was purified by C18 column (acetonitrile:water=5% to 100%) to give compound 13B (75 mg, 98.6% purity, 51.0% yield) as white solids.

Compound 13A

LC-MS (ESI): R_{T}=3.146 min, mass calcd. for C₂₇H₂₅Cl₂F₃N₆O₃ 608.1, m/z found 609.0 [M+H]⁺. Chiral HPLC (Column: Chiralpak IE 5 μm 4.6*250 mm; Mobile Phase: Hex:EtOH=30:70 at 1 mL/min; Temp: 30° C.; Wavelength: 254 nm, R_{T}=6.292 min). ¹H NMR (400 MHz, CDCl₃) δ 8.66 (s, 1H), 7.83 (d, J=7.6 Hz, 1H), 7.67 (d, J=8.4 Hz, 1H), 7.52 (d, J=9.2 Hz, 2H), 7.27-7.27 (m, 1H), 7.26-7.25 (m, 1H), 5.97-5.43 (m, 3H), 4.89-4.29 (m, 3H), 4.03-3.89 (m, 2H), 3.05 (br s, 1H), 2.76-2.64 (m, 4H), 1.67 (d, J=7.2 Hz, 3H), 1.30 (d, J=6.4 Hz, 3H). ¹⁹F NMR (376 MHz, CDCl₃) δ −67.90.

Compound 13B

LC-MS (ESI): R_{T}=3.261 min, mass calcd. for C₂₇H₂₅Cl₂F₃N₆O₃ 608.1, m/z found 609.0 [M+H]⁺. Chiral HPLC (Column: Chiralpak IE 5 μm 4.6*250 mm; Mobile Phase: Hex:EtOH=30:70 at 1 mL/min; Temp: 30° C.; Wavelength: 254 nm, R_{T}=8.681 min). ¹H NMR (400 MHz, CDCl₃) δ 8.74 (s, 1H), 7.88 (d, J=6.8 Hz, 1H), 7.68 (d, J=8.0 Hz, 1H), 7.54-7.52 (m, 2H), 7.27-7.27 (m, 1H), 7.25-7.25 (m, 1H), 6.01-5.42 (m, 3H), 4.88-4.44 (m, 3H), 4.20 (d, J=11.2 Hz, 1H), 3.48 (dd, J=12.8 and 4.8 Hz, 1H), 3.04 (br s, 1H), 2.82-2.70 (m, 4H), 1.69 (d, J=7.2 Hz, 3H), 1.33-1.25 (m, 3H). ¹⁹F NMR (376 MHz, CDCl₃) δ −67.96.

Compound 13B (Preparation Method B)

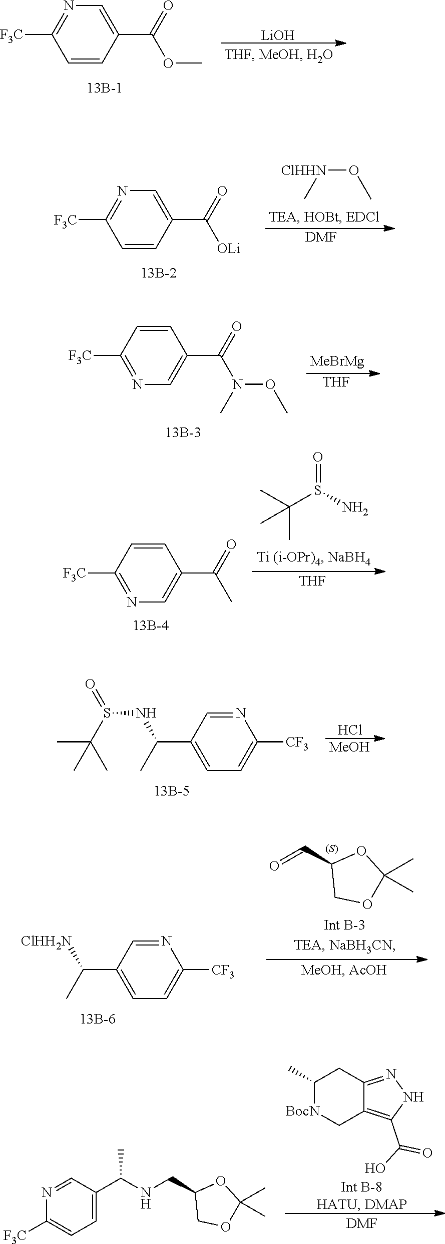

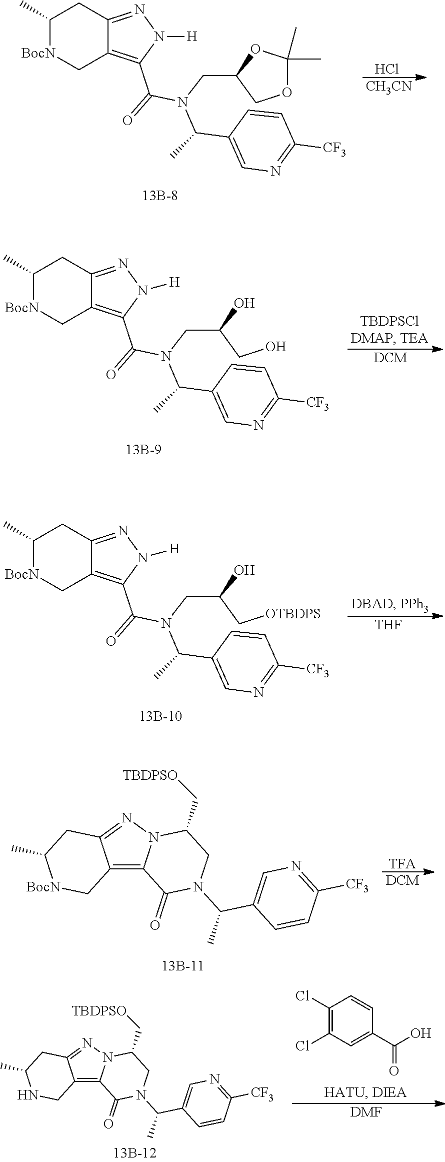

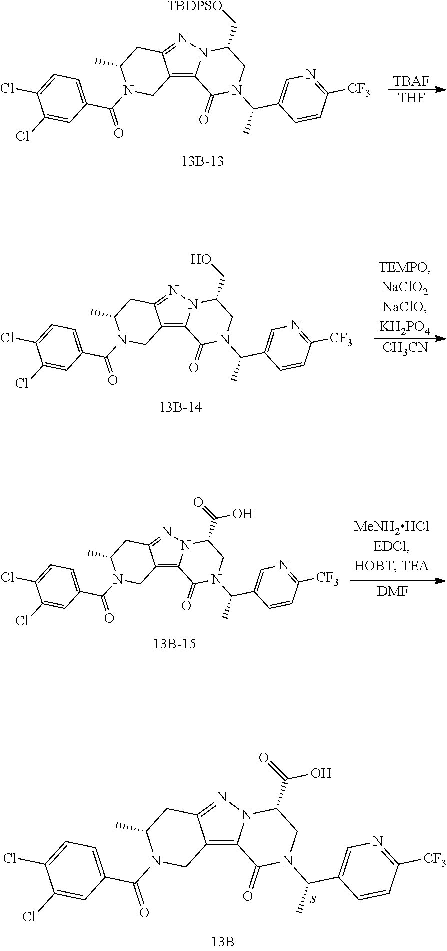

Intermediate 13B-2

Lithium 6-(trifluoromethyl)nicotinate

To the solution of methyl 6-(trifluoromethyl)nicotinate 13B-1 (40 g, 195 mmol) in tetrahydrofuran (50 mL), methanol (20 mL) and water (10 mL) was added lithium hydroxide monohydrate (10 g, 238 mmol) under nitrogen atmosphere. After stirred at room temperature for 3 hours, the reaction mixture was concentrated at room temperature to give the title compound (42 g, 90% purity, 98% yield) as white solids. ¹H NMR (400 MHz, DMSO-d₆) δ 9.14 (s, 1H), 8.41 (d, J=8.0 Hz, 1H), 7.83 (d, J=7.6 Hz, 1H).

Intermediate 13B-3

N-Methoxy-N-methyl-6-(trifluoromethyl)nicotinamide

To a solution of lithium 6-(trifluoromethyl)nicotinate 13B-2 (42 g, 90% purity, 192 mmol), N,O-dimethylhydroxylamine hydrochloride (18 g, 295 mmol), 1H-benzo[d][1,2,3]triazol-1-ol (39 g, 289 mmol) and triethylamine (39 g, 385 mmol) in N,N-dimethylformamide (300 mL) was added 1-ethyl-3-(3-dimethylaminopropyl)carbodiimide (55 g, 287 mmol) at room temperature under nitrogen atmosphere. After stirred at room temperature overnight, the mixture was diluted with water (120 mL) and extracted with ethyl acetate (130 mL) for three times. The combined organic layers were washed with brine (100 mL) for three times, dried over Na₂SO₄₍ₛ₎ and filtered. The filtrate was concentrated to give the title compound (45 g, 90% purity from ¹H NMR, 90% yield) as light yellow oil. ¹H NMR (400 MHz, CDCl₃) δ 9.03 (s, 1H), 8.21-8.18 (m, 1H), 7.73 (d, J=8.0 Hz, 1H), 3.55 (s, 3H), 3.40 (s, 3H).

Intermediate 13B-4

1-(6-(Trifluoromethyl)pyridin-3-yl)ethanone

To the solution of N-methoxy-N-methyl-6-(trifluoromethyl)nicotinamide 13B-3 (45 g, 90% purity, 172.948 mmol) in tetrahydrofuran (200 mL) was added 1 M methylmagnesium bromide in tetrahydrofuran (210 mL, 210 mmol) at 0° C. The mixture was stirred at 0° C. for 1 hour, then the reaction was quenched with ammonium chloride aqueous solution (100 mL) and extracted with ethyl acetate (100 mL) twice. The combined organic layers were washed with brine (100 mL), dried over Na₂SO₄₍ₛ₎ and filtered. The filtrate was concentrated to give the title compound (36 g, 90% purity from ¹H NMR, 99% yield) as yellow oil. ¹H NMR (400 MHz, CDCl₃) δ 9.25 (s, 1H), 8.43-8.41 (m, 1H), 7.82 (d, J=8.0 Hz, 1H), 2.70 (s, 3H).

Intermediate 13B-5

2-Methyl-N—((S)-1-(6-(trifluoromethyl)pyridin-3-yl)ethyl)propane-2-sulfinamide

To a solution of 1-(6-(trifluoromethyl)pyridin-3-yl)ethanone 13B-4 (13 g, 90% purity, 61.8 mmol) and (S)-2-methylpropane-2-sulfinamide (15 g, 123.8 mmol) in tetrahydrofuran (80 mL) was added titanium(IV) tetraisopropanolate (19 mL, 64.8 mmol). After stirred at 70° C. for 24 hours, the reaction mixture was cooled to 0° C., then sodium borohydride (2 g, 52.9 mmol) was added, the reaction mixture was stirred at 0° C. for 2 hours. The reaction was quenched with saturated ammonium chloride (80 mL) and filtered with kieselguhr. The cake was washed with ethyl acetate (80 mL). The filtrate was concentrated and purified by silica gel column chromatography (petroleum ether:ethyl acetate=1:1) to give the title compound (16 g, 90% purity from ¹H NMR, 79% yield, 99.7% stereopure) as yellow oil. Chiral analysis (Column: IC 5 μm 250 mm*4.6 mm; Mobile Phase: Hexane:EtOH=90:10 at 1 mL/min; Col. Temp: 30° C.; Wavelength: 254 nm, R_{T}=9.082 min). ¹H NMR (400 MHz, CDCl₃) δ 8.73 (s, 1H), 7.91-7.88 (m, 1H), 7.68 (d, J=8.0 Hz, 1H), 4.70-4.64 (m, 1H), 3.53 (s, 1H), 1.59 (d, J=6.4 Hz, 3H), 1.25 (s, 9H).

Intermediate 13B-6

(S)-1-(6-(Trifluoromethyl)pyridin-3-yl)ethanamine dihydrochloride

To a solution of (S)-2-methyl-N—((S)-1-(6-(trifluoromethyl)pyridin-3-yl)ethyl)propane-2-sulfinamide 13B-5 (16 g, 90% purity, 48.9 mmol) in methanol (50 mL) was added 4 M hydrochloride in methanol (20 mL, 80 mmol) slowly at 0° C. After stirred at 0° C. for 2 hours, the mixture was concentrated to give the title compound (15 g, 90% purity from ¹H NMR, 92% yield) as yellow oil. Chiral analysis (Column: IF 5 μm 250 mm*4.6 mm; Mobile Phase: Hex:EtOH=90:10 at 1 mL/min; Col. Temp: 30° C.; Wavelength: 254 nm, R_{T}=9.611 min, 98.7% stereopure). ¹H NMR (400 MHz, DMSO-d₆) δ 8.92 (s, 1H), 8.78 (br s. 2H), 8.29 (dd, J=8.0 and 2.0 Hz, 1H), 8.03 (d, J=8.0 Hz, 1H), 4.56-4.67 (m, 1H), 1.58 (d, J=6.8 Hz, 3H). In order to compare with literature reference compound, 13B-6 (3 g, 13.3 mmol) was dissolved in water (5 mL), then 1 M sodium hydroxide aqueous solution was added to adjust pH˜10 and extracted the solution with ethyl acetate (30 mL) for three times. The combined organic layers were washed with brine (30 mL), dried over anhydrous sodium sulfate, filtered and the filtrate was concentrated by reduced pressure to give the free base of 13B-6 (2 g, 67% yield, 98.5% stereopure) as colorless oil. LC-MS (ESI): R_{T}=1.06 min, mass calcd. for C₂₇H₃₆F₃N₅O₅ 190.2 m/z found 191.1 [M+H]⁺. Chiral analysis (Column: AD-H 5 μm 4.6 mm*250 mm; Mobile Phase: Hex:IPA:DEA=95:5:0.2 at 1 mL/min; Col. Temp: 30° C.; Wavelength: 254 nm, R_{T}: 9.723 min). Optical: −23.98° (c 0.5, CHCl₃) at [a]²⁰_{D} (Ref.: for R, +200 (c 0.5, CHCl₃) at [a]²⁰_{D}.

Intermediate 13B-7

(S)—N—(((R)-2,2-Dimethyl-1,3-dioxolan-4-yl)methyl)-1-(6-(trifluoromethyl)pyridin-3-yl)ethanamine

To a solution of (S)-1-(6-(trifluoromethyl)pyridin-3-yl)ethanamine dihydrochloride 13B-6 (4.00 g, 90% purity, 15.9 mmol) in methanol (40 mL) were added triethylamine (3.83 g, 37.8 mmol), (S)-2,2-dimethyl-1,3-dioxolane-4-carbaldehyde Int B-3 (5.00 g, 70% purity, 26.9 mmol) and acetic acid (2 mL) at room temperature. After stirred at room temperature for 2 hours, sodium cyanotrihydroborate (2.38 g, 37.9 mmol) was added into the mixture, then the stirring continued at room temperature for 1 hour under nitrogen atmosphere. The reaction mixture was quenched with water (50 mL) and extracted with ethyl acetate (50 mL) twice. The combined organic layers were washed with brine (50 mL), dried over Na₂SO₄₍ₛ₎ and filtered. The filtrate was concentrated and purified by silica gel column chromatography (petroleum ether:ethyl acetate=5:1 to 2:1) to give the title compound (3.90 g, 90% purity from ¹H NMR, 84% yield, 98.9% stereopure) as white solids. LC-MS (ESI): R_{T}=1.53 min, mass calcd. for C₁₄H₁₉F₃N₂O₂ 304.3, m/z found 305.2 [M+H]⁺. Chiral analysis (Column: Chiralpak IF 5 μm 4.6 mm*250 mm; Mobile Phase: Hex:EtOH=98:2 at 1 mL/min; Col. Temp: 30° C.; Wavelength: 254 nm, R_{T}=9.255 min). ¹H NMR (300 MHz, CDCl₃) b 8.70-8.64 (m, 1H), 7.92-7.87 (m, 1H), 7.65 (d, J=8.1 Hz, 1H), 4.25-4.14 (m, 1H), 4.03-3.89 (m, 3H), 3.57-3.52 (m, 1H), 2.68-2.61 (m, 1H), 2.47-2.42 (m, 1H), 1.40 (s, 6H), 1.35 (s, 3H).

Intermediate 13B-8

(R)-tert-Butyl 3-((((R)-2,2-dimethyl-1,3-dioxolan-4-yl)methyl)((S)-1-(6-(trifluoromethyl)pyridin-3-yl)ethyl)carbamoyl)-6-methyl-6,7-dihydro-2H-pyrazolo[4,3-c]pyridine-5(4H)-carboxylate

To a solution of (R)-5-(tert-butoxycarbonyl)-6-methyl-4,5,6,7-tetrahydro-2H-pyrazolo[4,3-c]pyridine-3-carboxylic acid Int B-8 (19.3 g, 95% purity, 65.2 mmol) in N,N-dimethylformamide (300 mL) were added N,N-dimethylpyridin-4-amine (18.6 g, 152 mmol), 2-(3H-[1,2,3]triazolo[4,5-b]pyridin-3-yl)-1,1,3,3-tetramethyluronium hexafluorophosphate (V) (33.1 g, 87.1 mmol) and (S)—N—(((R)-2,2-dimethyl-1,3-dioxolan-4-yl)methyl)-1-(6-(trifluoromethyl)pyridin-3-yl)ethanamine 13B-7 (14.7 g, 90% purity, 43.5 mmol) at room temperature under nitrogen atmosphere. After heated at 35° C. for 1 hour, then 45° C. for 1 hour and 55° C. overnight, the mixture was diluted with water (200 mL) and extracted with ethyl acetate (200 mL) for three times. The combined organic layers were washed with water (200 mL), dried over Na₂SO₄₍ₛ₎ and filtered. The filtrate was concentrated and purified by silica gel column chromatography (petroleum ether:ethyl acetate=5:1 to 2:1) to give the title compound (21.5 g, 58% purity from LCMS, 51% yield) as yellow solids. LC-MS (ESI): R_{T}=1.70 min, mass calcd. for C₂₇H₃₆F₃N₅O₅ 567.3, m/z found 568.4 [M+H]⁺.

Intermediate 13B-9

(R)-tert-Butyl 3-(((R)-2,3-dihydroxypropyl)((S)-1-(6-(trifluoromethyl)pyridin-3-yl)ethyl)carbamoyl)-6-methyl-6,7-dihydro-2H-pyrazolo[4,3-c]pyridine-5(4H)-carboxylate

To a solution of (R)-tert-butyl 3-((((R)-2,2-dimethyl-1,3-dioxolan-4-yl)methyl)((S)-1-(6-(trifluoromethyl)pyridin-3-yl)ethyl)carbamoyl)-6-methyl-6,7-dihydro-2H-pyrazolo[4,3-c]pyridine-5(4H)-carboxylate 13B-8 (17.0 g, 58% purity, 17.4 mmol) in acetonitrile (170 mL) was added 2 M hydrochloride aqueous solution (170 mL, 340 mmol) at 0° C. under nitrogen atmosphere. After stirred at 0° C. for 1 hour, the reaction mixture was added saturated sodium bicarbonate aqueous solution (200 mL) and extracted with ethyl acetate (200 mL) for three times. The combined organic layers were washed with brine (500 mL), then dried over Na₂SO₄₍ₛ₎, concentrated and purified by silica gel column chromatography (petroleum ether: ethyl acetate=10:1 to 1:2) to give the title compound (10.5 g, 82% purity from LCMS, 94% yield) as white solids. LC-MS (ESI): R_{T}=1.39 min, mass calcd. for C₂₄H₃₂F₃N₅O₅ 527.2, m/z found 528.6 [M+H]⁺.

Intermediate 13B-10

tert-butyl (R)-3-(((R)-3-((tert-butyldiphenylsilyl)oxy)-2-hydroxypropyl)((S)-1-(6-(trifluoromethyl)pyridin-3-yl)ethyl)carbamoyl)-6-methyl-2,4,6,7-tetrahydro-5H-pyrazolo[4,3-c]pyridine-5-carboxylate

To a solution of (R)-tert-butyl 3-(((R)-2,3-dihydroxypropyl)((S)-1-(6-(trifluoromethyl)pyridin-3-yl)ethyl)carbamoyl)-6-methyl-6,7-dihydro-2H-pyrazolo[4,3-c]pyridine-5(4H)-carboxylate 13B-9 (16.7 g, 82% purity, 26.0 mmol) in dichloromethane (167 mL) was added triethylamine (5.3 g, 52.4 mmol), N,N-dimethylpyridin-4-amine (3.5 g, 28.6 mmol) and tert-butylchlorodiphenylsilane (8.56 g, 31.1 mmol) at 0° C. under nitrogen atmosphere. After stirred at room temperature for 1 hour, the reaction mixture was added water (300 mL) and extracted with dichloromethane (100 mL) for three times. The combined organic layers were washed with brine (300 mL), then dried over Na₂SO₄₍ₛ₎, concentrated and purified by silica gel column chromatography (petroleum ether:ethyl acetate=10:1 to 1:1) to give the title compound (20 g, 97% purity from LCMS, 98% yield) as white solids. LC-MS (ESI): R_{T}=1.61 min, mass calcd. for C₄₀H₅₀F₃N₅O₅Si 765.4, m/z found 766.5 [M+H]⁺.

Intermediate 13B-11

(3R,7S)-tert-butyl 7-(((tert-butyldiphenylsilyl)oxy)methyl)-3-methyl-10-oxo-9-((S)-1-(6-(trifluoromethyl)pyridin-3-yl)ethyl)-3,4,7,8,9,10-hexahydropyrido[4′,3′:3,4]pyrazolo[1,5-a]pyrazine-2(1H)-carboxylate

To a solution of tert-butyl (R)-3-(((R)-3-((tert-butyldiphenylsilyl)oxy)-2-hydroxypropyl)((S)-1-(6-(trifluoromethyl)pyridin-3-yl)ethyl)carbamoyl)-6-methyl-2,4,6,7-tetrahydro-5H-pyrazolo[4,3-c]pyridine-5-carboxylate 13B-10 (21 g, 97% purity, 26.6 mmol) in tetrahydrofuran (210 mL) were added triphenylphosphine (17.4 g, 66.3 mmol) and di-tert-butyl diazene-1,2-dicarboxylate (15.3 g, 66.4 mmol) at 0° C. under nitrogen atmosphere. After stirred at 0° C. for 2 hours, the reaction mixture was added water (200 mL) and extracted with ethyl acetate (200 mL) for three times. The combined organic layers were washed with brine (300 mL) then dried over Na₂SO₄₍ₛ₎, concentrated and purified by silica gel column chromatography (petroleum ether:ethyl acetate=10:1 to 1:1) to give the title compound (30 g, 45% purity from ¹HNMR, 68% yield) as white solids. LC-MS (ESI): R_{T}=1.26 min, mass calcd. for C₄₀H₄₈F₃N₅O₄Si 747.3, m/z found [M+H]+ 748.6. ¹H NMR (400 MHz, CDCl₃) δ 8.75 (s, 1H), 7.85 (d, J=6.4 Hz, 1H), 7.68 (d, J=8.4 Hz, 1H), 7.60-7.56 (m, 4H), 7.44-7.38 (m, 6H), 6.17-6.14 (m, 1H), 5.16-5.06 (m, 1H), 4.91-4.80 (m, 1H), 4.39-4.33 (m, 1H), 4.18-4.08 (m, 2H), 3.83 (t, J=9.6 Hz, 1H), 3.73-3.68 (m, 1H), 3.39-3.34 (m, 1H), 2.93-2.88 (m, 1H), 2.54-2.50 (m, 1H), 1.62 (d, J=7.2 Hz, 3H), 1.47 (s, 9H), 1.07 (d, J=6.8 Hz, 3H), 1.03 (s, 9H).

Intermediate 13B-12

(3R,7S)-7-(((tert-butyldiphenylsilyl)oxy)methyl)-3-methyl-9-((S)-1-(6-(trifluoromethyl)pyridin-3-yl)ethyl)-1,2,3,4,8,9-hexahydropyrido[4′,3′:3,4]pyrazolo[1,5-a]pyrazin-10(7H)-one

To a solution of (3R,7S)-tert-butyl 7-(((tert-butyldiphenylsilyl)oxy)methyl)-3-methyl-10-oxo-9-((S)-1-(6-(trifluoromethyl)pyridin-3-yl)ethyl)-3,4,7,8,9,10-hexahydropyrido [4′,3′:3,4]pyrazolo[1,5-a]pyrazine-2(1H)-carboxylate 13B-11 (15 g, 45% purity, 9.03 mmol) in dichloromethane (75 mL) was added trifluoroacetic acid (37 mL) under nitrogen atmosphere. After stirred at 0° C. for 1 hour, the reaction mixture was added saturated sodium bicarbonate aqueous solution (300 mL) and extracted with dichloromethane (100 mL) for three times. The combined organic layers were washed with brine (300 mL), then dried over Na₂SO₄₍ₛ₎, concentrated and purified by silica gel column chromatography (petroleum ether: ethyl acetate=10:1 to 1:10) to give the title compound (5.7 g, 100% purity from LCMS, 97% yield) as white solids. LC-MS (ESI): R_{T}=1.14 min, mass calcd. for C₃₅H₄₀F₃N₅O₂Si 647.3, m/z found 648.4 [M+H]⁺. ¹H NMR (400 MHz, CDCl₃) δ 8.74 (s, 1H), 7.85-7.83 (m, 1H), 7.67 (d, J=8.4 Hz, 1H), 7.59-7.56 (m, 4H), 7.46-7.36 (m, 6H), 6.15-6.10 (m, 1H), 4.35-4.27 (m, 2H), 4.15-4.08 (m, 2H), 4.01 (d, J=16.4 Hz, 1H), 3.77 (t, J=9.6 Hz, 1H), 3.71-3.66 (m, 1H), 3.39-3.34 (m, 1H), 2.98-2.94 (m, 1H), 2.75-2.70 (m, 1H), 2.30-2.23 (m, 1H), 1.59 (d, J=6.8 Hz, 3H), 1.25 (d, J=5.6 Hz, 3H), 1.03 (s, 9H).

Intermediate 13B-13

(3R,7S)-7-(((tert-butyldiphenylsilyl)oxy)methyl)-2-(3,4-dichlorobenzoyl)-3-methyl-9-((S)-1-(6-(trifluoromethyl)pyridin-3-yl)ethyl)-1,2,3,4,8,9-hexahydropyrido [4′,3′:3,4]pyrazolo[1,5-a]pyrazin-10(7H)-one

To the solution of 3,4-dichlorobenzoic acid (4.48 g, 23.5 mmol) in N,N-dimethylformamide (100 mL) were added N-ethyl-N-isopropylpropan-2-amine (9.5 g, 73.5 mmol), 2-(3H-[1,2,3]triazolo[4,5-b]pyridin-3-yl)-1,1,3,3-tetramethylisouronium hexafluorophosphate (V) (11.2 g, 29.5 mmol) and (3R,7S)-7-(((tert-butyldiphenylsilyl)oxy)methyl)-3-methyl-9-((S)-1-(6-(trifluoromethyl)pyridin-3-yl)ethyl)-1,2,3,4,8,9-hexahydropyrido[4′,3′:3,4]pyrazolo[1,5-a]pyrazin-10(7H)-one 13B-12 (9.5 g, 100% purity, 14.7 mmol) at 0° C. under nitrogen atmosphere. After stirred at 0° C. for 1 hour, the reaction mixture was added water (200 mL) and extracted with ethyl acetate (200 mL) for three times. The combined organic layers were washed with brine (300 mL) then dried over Na₂SO₄₍ₛ₎, concentrated and purified by silica gel column chromatography (petroleum ether:ethyl acetate=10:1 to 2:1) to give the title compound (11.9 g, 100% purity from LCMS, 99% yield) as white solids. LC-MS (ESI): R_{T}=1.30 min, mass calcd. for C₄₂H₄₂Cl₂F₃N₅O₃Si 819.2, m/z found [M+H]+no Ms. ¹H NMR (400 MHz, CDCl₃) δ 8.72 (s, 1H), 7.90-7.77 (m, 1H), 7.68 (d, J=8.0 Hz, 1H), 7.60-7.56 (m, 4H), 7.52-7.38 (m, 8H), 7.25-7.23 (m, 1H), 6.19-6.01 (m, 1H), 5.67-4.80 (m, 2H), 4.49-4.30 (m, 2H), 4.10-4.07 (m, 1H), 3.83 (t, J=9.6 Hz, 1H), 3.75-3.69 (m, 1H), 3.39-3.35 (m, 1H), 3.04-2.92 (m, 1H), 2.64-2.55 (m, 1H), 1.61 (d, J=6.8 Hz, 3H), 1.20-1.16 (m, 3H), 1.03 (s, 9H).

Intermediate 13B-14

(3R,7S)-2-(3,4-dichlorobenzoyl)-7-(hydroxymethyl)-3-methyl-9-((S)-1-(6-(trifluoromethyl)pyridin-3-yl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-9-((S)-1-(6-(trifluoromethyl)pyridin-3-yl)ethyl)-1,2,3,4,8,9-hexahydropyrido[4′,3′:3,4]pyrazolo[1,5-a]pyrazin-10(7H)-one 13B-13 (12.9 g, 100% purity, 15.7 mmol) in tetrahydrofuran (129 mL) was added 1 M tetrabutylammonium fluoride in tetrahydrofuran (21.5 mL, 21.5 mmol) dropwise and the reaction mixture was stirred at 0° C. for 1 hour. Then the reaction mixture was added water (300 mL) and extracted with ethyl acetate (150 mL) for three times. The combined organic layers were washed with brine (300 mL), then dried over Na₂SO₄₍ₛ₎, concentrated and purified by silica gel column chromatography (methanol:ethyl acetate=1:100 to 1:20) to give the title product (9.15 g, 100% purity from LCMS, 99% yield) as white solids. LC-MS (ESI): R_{T}=1.61 min, mass calcd. for C₂₆H₂₄Cl₂F₃N₅O₃ 581.1, m/z found 582.4 [M+H]⁺. ¹H NMR (400 MHz, CDCl₃) δ 8.73 (s, 1H), 7.87-7.82 (m, 1H), 7.70 (d, J=8.0 Hz, 1H), 7.54-7.51 (m, 2H), 7.28-7.27 (m, 1H), 6.23-6.05 (m, 1H), 5.71-4.80 (m, 2H), 4.51-4.31 (m, 2H), 4.06-3.96 (m, 2H), 3.71-3.66 (m, 1H), 3.27-3.17 (m, 1H), 3.11-3.03 (m, 1H), 2.93-2.87 (m, 1H), 2.72-2.64 (m, 1H), 1.68 (d, J=6.8 Hz, 3H), 1.25 (d, J=7.2 Hz, 3H).

Intermediate 13B-15

(3R,7S)-2-(3,4-dichlorobenzoyl)-3-methyl-10-oxo-9-((S)-1-(6-(trifluoromethyl)pyridin-3-yl)ethyl)-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-7-(hydroxymethyl)-3-methyl-9-((S)-1-(6-(trifluoromethyl)pyridin-3-yl)ethyl)-1,2,3,4,8,9-hexahydropyrido[4′,3′:3,4]pyrazolo [1,5-a]pyrazin-10(7H)-one 13B-14 (4.4 g, 99.2% purity, 7.49 mmol) in acetonitrile (44 mL) were added saturated potassium dihydrogenphosphate aqueous (44 mL), sodium chlorite (1.71 g, 80% purity, 15.1 mmol), 2,2,6,6-tetramethylpiperidinooxy (2.37 g, 15.2 mmol) and sodium hypochlorite (9 mL, 10% purity, 15.1 mmol). After stirred at 0° C. for 6 hours, the reaction mixture was added water (50 mL) and extracted with ethyl acetate (50 mL) for three times. The combined organic layers were washed with brine (60 mL) then dried Na₂SO₄ (s), concentrated and triturated with ACN (90 mL) to give the title product (4.4 g, 100% purity from LCMS, 98% yield) as white solids. LC-MS (ESI): R_{T}=1.22 min, mass calcd. for C₂₆H₂₂C₁₂F₃N₅O₄ 595.1, m/z found 596.2 [M+H]⁺.

Compound 13B

(3R,7S)-2-(3,4-dichlorobenzoyl)-N,3-dimethyl-10-oxo-9-((S)-1-(6-(trifluoromethyl) pyridin-3-yl)ethyl)-1,2,3,4,7,8,9,10-octahydropyrido[4′,3′:3,4]pyrazolo[1,5-a]pyrazine-7-carboxamide

To the solution of (3R,7S)-2-(3,4-dichlorobenzoyl)-3-methyl-10-oxo-9-((S)-1-(6-(trifluoromethyl)pyridin-3-yl)ethyl)-1,2,3,4,7,8,9,10-octahydropyrido[4′,3′:3,4]pyrazolo[1,5-a]pyrazine-7-carboxylic acid 13B-15 (4.4 g, 100% purity, 7.38 mmol), methylamine hydrochloride (1.32 g, 19.6 mmol), N1-((ethylimino)methylene)-N3,N3-dimethylpropane-1,3-diamine hydrochloride (3.0 g, 15.6 mmol) and 1-Hydroxybenzotrizole (2.11 g, 15.6 mmol) in N,N-dimethylformamide (147 mL) was added triethylamine (5.1 g, 50.4 mmol) dropwise at 0° C. under nitrogen atmosphere. After stirred at 0° C. for 1 hour, the mixture was diluted with water (300 mL) and extracted with ethyl acetate (300 mL) for three times. The combined organic layers were washed with 1 M hydrochloride aqueous solution (50 mL) and brine (300 mL), dried over Na₂SO₄₍ₛ₎ and filtered. The filtrate was concentrated and purified by C18 column (acetonitrile:water (0.1% ammonium bicarbonate)=5% to 95%) to give the title compound (2.82 g, 99.6% purity from LCMS, 64% yield, 99.7% stereopure) as white solids. LC-MS (ESI): R_{T}=8.142 min, mass calcd. for C₂₇H₂₅C₁₂F₃N₆O₃ 608.1, m/z found 609.1 [M+H]⁺. Chiral analysis (Column: Chiralpak IC 5 μm 4.6 mm*250 mm; Mobile Phase: ACN:IPA=80:20 at 1 mL/min; Col. Temp: 30° C.; Wavelength: 254 nm, R_{T}=5.378 min). ¹H NMR (400 MHz, CDCl₃) δ 8.74 (s, 1H), 7.92-7.84 (m, 1H), 7.68 (d, J=8.0 Hz, 1H), 7.54-7.51 (m, 2H), 7.27-7.25 (m, 2H), 6.12-5.28 (m, 3H), 4.92-4.43 (m, 3H), 4.24-4.13 (m, 1H), 3.50-3.46 (m, 1H), 3.12-2.97 (m, 1H), 2.81 (d, J=5.2 Hz, 3H), 2.73-2.66 (m, 1H), 1.68 (d, J=7.2 Hz, 3H), 1.29 (d, J=6.8 Hz, 3H).

Compounds 14A and 14B

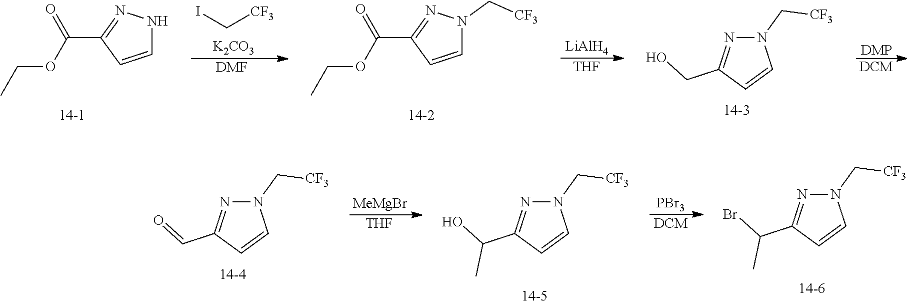

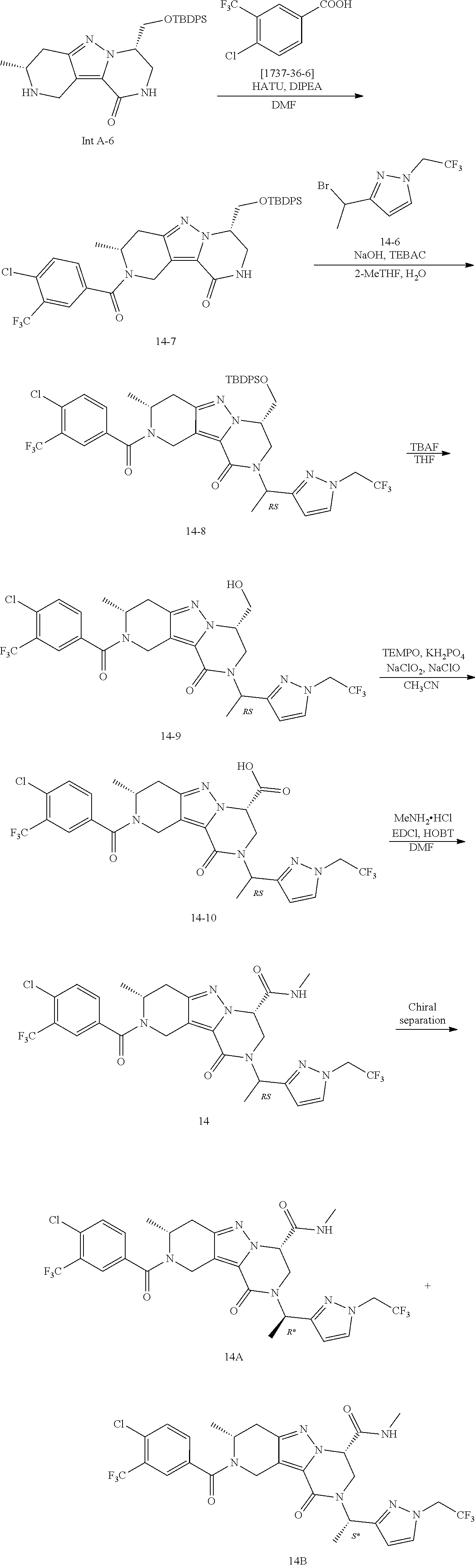

Intermediate 14-2

Ethyl 1-(2,2,2-trifluoroethyl)-1H-pyrazole-3-carboxylate

To a solution of ethyl pyrazole-3-carboxylate 14-1 (2 g, 14.3 mmol) and 2-iodo-1,1,1-trifluoroethane (2.4 mL, 24.4 mmol) in N,N-dimethylformamide (30 mL) was added potassium carbonate (6 g, 43.4 mmol). After being stirred at 100° C. for 5 hours, the mixture was cooled down, diluted with water (80 mL), and extracted with ethyl acetate (30 mL) twice. The combined organic layers were dried over Na₂SO₄₍ₛ₎, and filtered. The filtrate was concentrated and purified by C18 column (acetonitrile:water=50% to 60%) to give the title compound (1.68 g, 100% purity from LCMS, 53% yield) as a colorless oil. LC-MS (ESI): R_{T}=1.47 min, mass calcd. for C₈H₉F₃N₂O₂ 222.1, m/z found 223.0 [M+H]⁺. ¹H NMR (400 MHz, CDCl₃) δ 7.56 (d, J=2.0 Hz, 1H), 6.91 (d, J=2.4 Hz, 1H), 4.81 (q, J=8.4 Hz, 2H), 4.42 (q, J=7.2 Hz, 2H), 1.41 (t, J=7.2 Hz, 3H).

Intermediate 14-3

(1-(2,2,2-Trifluoroethyl)-1H-pyrazol-3-yl)methanol

To a solution of ethyl 1-(2,2,2-trifluoroethyl)-1H-pyrazole-3-carboxylate 14-2 (1.6 g, 90% purity, 6.48 mmol) in tetrahydrofuran (20 mL) was added lithium aluminum hydride (300 mg, 7.90 mmol) at 0° C. After being stirred at room temperature for 1 hour, the reaction mixture was quenched with sodium sulfate decahydrate (800 mg), and filtered. The filtrate was concentrated to give the title compound (1.1 g, 94% purity from LCMS, 89% yield) as a colorless oil. LC-MS (ESI): R_{T}=0.96 min, mass calcd. for C₆H₇F₃N₂O 180.1, m/z found 181.0 [M+H]⁺. ¹H NMR (400 MHz, CDCl₃) δ 7.47 (d, J=2.0 Hz, 1H), 6.36 (d, J=2.4 Hz, 1H), 4.71-4.64 (m, 4H), 2.21 (t, J=6.0 Hz, 1H).

Intermediate 14-4

1-(2,2,2-Trifluoroethyl)-1H-pyrazole-3-carbaldehyde

To a solution of (1-(2,2,2-trifluoroethyl)-1H-pyrazol-3-yl)methanol 14-3 (1.1 g, 94% purity, 5.74 mmol) in dichloromethane (20 mL) was added dess-martin periodinane (3 g, 7.07 mmol) at 0° C. After being stirred at room temperature for 3 hours, the mixture was quenched with saturated sodium thiosulfate aqueous solution (20 mL), and extracted with dichloromethane (20 mL) twice. The combined organic layers were washed with saturated sodium bicarbonate aqueous solution (20 mL) and brine (10 mL), dried over Na₂SO₄₍ₛ₎, and filtered. The filtrate was concentrated to give the title compound (1.8 g, 50% purity from ¹H NMR, 88% yield) as white solids. ¹H NMR (400 MHz, CDCl₃) δ 10.00 (d, J=0.8 Hz, 1H), 7.59 (d, J=2.8 Hz, 1H), 6.90 (d, J=2.4 Hz, 1H), 4.82 (q, J=8.4 Hz, 2H).

Intermediate 14-5

1-(1-(2,2,2-Trifluoroethyl)-1H-pyrazol-3-yl)ethanol

To a solution of 1-(2,2,2-trifluoroethyl)-1H-pyrazole-3-carbaldehyde 14-4 (1.8 g, 50% purity, 5.05 mmol) in tetrahydrofuran (20 mL) was added methylmagnesium bromide (3.6 mL, 3 M in 2-methyltetrahydrofuran, 10.8 mmol) at 0° C. under nitrogen atmosphere. It was warmed to room temperature gradually and stirred for 3 hours. The mixture was quenched with water (10 mL), and extracted with ethyl acetate (15 mL) three times. The combined organic layers were dried over Na₂SO₄₍ₛ₎, and filtered. The filtrate was concentrated and purified by C18 column (acetonitrile:water=20% to 40%) to give the title compound (570 mg, 90% purity from ¹H NMR, 52% yield) as a colorless oil. ¹H NMR (400 MHz, CDCl₃) δ 7.45 (d, J=2.0 Hz, 1H), 6.32 (d, J=2.4 Hz, 1H), 4.96 (q, J=6.4 Hz, 1H), 4.66 (q, J=8.4 Hz, 2H), 2.30 (br s, 1H), 1.53 (d, J=6.8 Hz, 3H).

Intermediate 14-6

3-(1-Bromoethyl)-1-(2,2,2-trifluoroethyl)-1H-pyrazole

To a solution of 1-(1-(2,2,2-trifluoroethyl)-1H-pyrazol-3-yl)ethanol 14-5 (500 mg, 90% purity, 2.32 mmol) in dichloromethane (5 mL) was added phosphorus tribromide (400 mg, 1.48 mmol) at 0° C. After being stirred at room temperature for 1 hour, the mixture was quenched with saturated sodium bicarbonate aqueous solution (10 mL), and extracted with dichloromethane (10 mL) twice. The combined organic layers were washed with brine (5 mL), dried over Na₂SO₄₍ₛ₎, and filtered. The filtrate was concentrated to give the title compound (640 mg, 90% purity from ¹H NMR, 97% yield) as a brown oil. ¹H NMR (400 MHz, CDCl₃) δ 7.45 (d, J=1.6 Hz, 1H), 6.44 (d, J=2.4 Hz, 1H), 5.27 (q, J=7.2 Hz, 1H), 4.66 (dq, J=8.4, 1.2 Hz, 2H), 2.05 (d, J=6.8 Hz, 3H).

Intermediate 14-7

(3R,7S)-7-(((tert-Butyldiphenylsilyl)oxy)methyl)-2-(4-chloro-3-(trifluoromethyl)benzoyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-3-methyl-1,2,3,4,8,9-hexahydropyrido[4′,3′:3,4]pyrazolo[1,5-a]pyrazin-10(7H)-one Int A-6 (650 mg, 78% purity, 1.07 mmol) and 4-chloro-3-(trifluoromethyl)benzoic acid (312 mg, 1.39 mmol) in N,N-dimethylformamide (6 mL) was added 2-(3H-[1,2,3]triazolo[4,5-b]pyridin-3-yl)-1,1,3,3-tetramethyluronium hexafluorophosphate(V) (568 mg, 1.50 mmol) and N,N-diisopropylethylamine (415 mg, 3.21 mmol). After being stirred at 25° C. for overnight, the mixture was concentrated to give residue. The residue was purified by silica gel column chromatography (dichloromethane:methanol=100:1) to give the title compound (700 mg, 94% purity from LCMS, 90% yield) as white solids. LC-MS (ESI): R_{T}=2.04 min, mass calcd. for C₃₅H₃₆ClF₃N₄O₃Si 680.2, m/z found 680.8 [M+H]⁺. ¹H NMR (400 MHz, DMSO-d₆) 8.18-8.12 (m, 1H), 7.92-7.79 (m, 3H), 7.56-7.40 (m, 10H), 5.43-5.24 (m, 1H), 4.58-4.49 (m, 2H), 4.16-4.12 (m, 1H), 3.99-3.69 (m, 4H), 3.00-2.95 (m, 1H), 2.68-2.56 (m, 1H), 1.09 (br s, 3H), 0.92 (s, 9H).

Intermediate 14-8

(3R,7S)-7-(((tert-Butyldiphenylsilyl)oxy)methyl)-2-(4-chloro-3-(trifluoromethyl)benzoyl)-3-methyl-9-(1-(1-(2,2,2-trifluoroethyl)-1H-pyrazol-3-yl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(4-chloro-3-(trifluoromethyl)benzoyl)-3-methyl-1,2,3,4,8,9-hexahydropyrido[4′,3′:3,4]pyrazolo[1,5-a]pyrazin-10(7H)-one 14-7 (500 mg, 100% purity, 0.734 mmol) and 3-(1-bromoethyl)-1-(2,2,2-trifluoroethyl)-1H-pyrazole 14-6 (400 mg, 90% purity, 1.40 mmol) in 2-methyltetrahydrofuran (5 mL) was added 50% wt. sodium hydroxide aqueous solution (5 mL) and benzyltriethylammonium chloride (20 mg, 0.088 mmol). After being stirred at 20° C. for 5 hours, the reaction mixture was diluted with water (5 mL), and extracted with ethyl acetate (5 mL) three times. The combined organic layers were washed with brine (5 mL), dried over Na₂SO₄₍ₛ₎, and filtered. The filtrate was concentrated to give a mixture of P1 and P2 (800 mg, 55% purity from LCMS, 70% yield) as yellow solids. LC-MS (ESI): R_{T}=2.04 min and 2.10 min, mass calcd. for C₄₂H₄₃ClF₆N₆O₃Si 856.3, m/z found 857.1 [M+H]⁺.

Intermediate 14-9

(3R,7S)-2-(4-Chloro-3-(trifluoromethyl)benzoyl)-7-(hydroxymethyl)-3-methyl-9-(1-(1-(2,2,2-trifluoroethyl)-1H-pyrazol-3-yl)ethyl)-1,2,3,4,8,9-hexahydropyrido[4′,3′:3,4]pyrazolo[1,5-a]pyrazin-10(7H)-one

To the solution of (3R,7S)-7-(((tert-butyldiphenylsilyl)oxy)methyl)-2-(4-chloro-3-(trifluoromethyl)benzoyl)-3-methyl-9-(1-(1-(2,2,2-trifluoroethyl)-1H-pyrazol-3-yl)ethyl)-1,2,3,4,8,9-hexahydropyrido[4′,3′:3,4]pyrazolo[1,5-a]pyrazin-10(7H)-one 14-8 (900 mg, 55% purity, 0.577 mmol) in tetrahydrofuran (5 mL) was added tetrabutylammonium fluoride solution (1 mL, 1 M in tetrahydrofuran, 1 mmol). After being stirred at room temperature for 1 hour, the reaction mixture was concentrated, and purified by purified by silica gel column chromatography (dichloromethane:methanol=10:1) and C18 column (acetonitrile:water=50% to 65%) to give the title compound (320 mg, 100% purity from LCMS, 90% yield) as white solids. LC-MS (ESI): R_{T}=1.59 min, mass calcd. for C₂₆H₂₅ClF₆N₆O₃ 618.2, m/z found 619.0 [M+H]⁺.

Intermediate 14-10

(3R,7S)-2-(4-Chloro-3-(trifluoromethyl)benzoyl)-3-methyl-10-oxo-9-(1-(1-(2,2,2-trifluoroethyl)-1H-pyrazol-3-yl)ethyl)-1,2,3,4,7,8,9,10-octahydropyrido[4′,3′:3,4]pyrazolo[1,5-a]pyrazine-7-carboxylic acid

To a solution of (3R,7S)-2-(4-chloro-3-(trifluoromethyl)benzoyl)-7-(hydroxymethyl)-3-methyl-9-(1-(1-(2,2,2-trifluoroethyl)-1H-pyrazol-3-yl)ethyl)-1,2,3,4,8,9-hexahydropyrido[4′,3′:3,4]pyrazolo[1,5-a]pyrazin-10(7H)-one 14-9 (320 mg, 100% purity, 0.517 mmol), sodium chlorite (120 mg, 1.06 mmol) and 2,2,6,6-tetramethylpiperidinooxy (160 mg, 1.02 mmol) in acetonitrile (5 mL) was added saturated aqueous potassium phosphate monobasic solution (5 mL) and sodium hypochlorite solution (0.6 mL, 10% aqueous solution, 1.01 mmol) at 0° C. After being stirred at room temperature overnight, the reaction mixture was quenched with saturated aqueous sodium sulfite solution (10 mL), acidized with 1N hydrochloride to pH˜4, and extracted with ethyl acetate (10 mL) twice. The combined organic layers were dried over Na₂SO₄₍ₛ₎, and filtered. The filtrate was concentrated, and purified by C18 column (acetonitrile:water=40% to 60%) to give the title compound (330 mg, 95% purity from LCMS, 96% yield) as white solids. LC-MS (ESI): R_{T}=1.31 min, mass calcd. for C₂₆H₂₃ClF₆N₆O₄ 632.1, m/z found 633.0 [M+H]⁺.

Compound 14

(3R,7S)-2-(4-Chloro-3-(trifluoromethyl)benzoyl)-N,3-dimethyl-10-oxo-9-(1-(1-(2,2,2-trifluoroethyl)-1H-pyrazol-3-yl)ethyl)-1,2,3,4,7,8,9,10-octahydropyrido[4′,3′:3,4]pyrazolo[1,5-a]pyrazine-7-carboxamide

To the solution of (3R,7S)-2-(4-chloro-3-(trifluoromethyl)benzoyl)-3-methyl-10-oxo-9-(1-(1-(2,2,2-trifluoroethyl)-1H-pyrazol-3-yl)ethyl)-1,2,3,4,7,8,9,10-octahydropyrido[4′,3′:3,4]pyrazolo[1,5-a]pyrazine-7-carboxylic acid 14-10 (330 mg, 95% purity, 0.495 mmol) and methylamine hydrochloride (80 mg, 1.13 mmol) in N,N-dimethylformamide (5 mL) was added triethylamine (0.3 mL, 2.16 mmol), 3-(ethyliminomethylideneamino)-N,N-dimethylpropan-1-amine hydrochloride (150 mg, 0.782 mmol) and 1-hydroxybenzotrizole (110 mg, 0.814 mmol) at 0° C. After being stirred at room temperature overnight, the mixture was diluted with water (10 mL), and extracted with ethyl acetate (10 mL) twice. The combined organic layers were concentrated, and purified by C18 column (acetonitrile:water=45% to 60%) to give the title compound (150 mg, 92% purity from LCMS, 43% yield) as white solids. LC-MS (ESI): R_{T}=1.57 min, mass calcd. for C₂₇H₂₆ClF₆N₇O₃ 645.2, m/z found 646.1 [M+H]⁺.

Compounds 14A and 14B

(3R,7S)-2-(4-chloro-3-(trifluoromethyl)benzoyl)-N,3-dimethyl-10-oxo-9-((R*)-1-(1-(2,2,2-trifluoroethyl)-1H-pyrazol-3-yl)ethyl)-1,2,3,4,7,8,9,10-octahydropyrido[4′,3′:3,4]pyrazolo[1,5-a]pyrazine-7-carboxamide (14A), and (3R,7S)-2-(4-chloro-3-(trifluoromethyl)benzoyl)-N,3-dimethyl-10-oxo-9-((S*)-1-(1-(2,2,2-trifluoroethyl)-1H-pyrazol-3-yl)ethyl)-1,2,3,4,7,8,9,10-octahydropyrido[4′,3′:3,4]pyrazolo[1,5-a]pyrazine-7-carboxamide (14B)

A racemic of (3R,7S)-2-(4-chloro-3-(trifluoromethyl)benzoyl)-N,3-dimethyl-10-oxo-9-(1-(1-(2,2,2-trifluoroethyl)-1H-pyrazol-3-yl)ethyl)-1,2,3,4,7,8,9,10-octahydropyrido[4′,3′:3,4]pyrazolo[1,5-a]pyrazine-7-carboxamide compound 14 (150 mg, 92% purity, 0.214 mmol) was separated by chiral Prep. HPLC (separation condition: Column: Chiralpak IA 5 um 30*250 mm; Mobile Phase: Hex:EtOH=40:60, at 55 mL/min; Col. Temp: 30° C.; Wavelength: 230 nm) to afford the crude P1 and crude P2. The crude P1 was purified by C18 column (acetonitrile:water(+0.2% ammonium bicarbonate)=5% to 95%) to give compound 14A (35 mg, 99.5% purity from LCMS, 25% yield) as white solids. The crude P2 was purified by C18 column (acetonitrile:water (+0.2% ammonium bicarbonate)=5% to 95%) to give compound 14B (65 mg, 99.8% purity from LCMS, 47% yield) as white solids.

Compound 14A

LC-MS (ESI): R_{T}=3.821 min, mass calcd. for C₂₇H₂₆ClF₆N₇O₃ 645.2, m/z found 646.4 [M+H]⁺. Chiral HPLC (Column: Chiralpak IA 5 μm 4.6*250 mm; Mobile Phase: Hex:EtOH=40:60 at 1 mL/min; Temp: 30° C.; Wavelength: 230 nm), R_{T}=4.742 min). ¹H NMR (400 MHz, CDCl₃) δ 7.78 (d, J=1.2 Hz, 1H), 7.58 (d, J=8.0 Hz, 1H), 7.55-7.52 (m, 1H), 7.46 (d, J=2.0 Hz, 1H), 7.26-7.25 (m, 1H), 6.21 (d, J=1.6 Hz, 1H), 6.10-5.42 (m, 3H), 4.86-4.33 (m, 4H), 4.01-3.88 (m, 2H), 3.08 (br s, 1H), 2.75-2.71 (m, 4H), 1.59-1.57 (m, 3H), 1.31 (d, J=6.4 Hz, 3H). ¹⁹F NMR (376 MHz, CDCl₃) δ −62.82, −71.64.

Compound 14B

LC-MS (ESI): R_{T}=3.015 min, mass calcd. for C₂₇H₂₆ClF₆N₇O₃ 645.2, m/z found 646.3 [M+H]⁺. Chiral HPLC (Column: Chiralpak IA 5 μm 4.6*250 mm; Mobile Phase: Hex:EtOH=40:60 at 1 mL/min; Temp: 30° C.; Wavelength: 230 nm), R_{T}=6.612 min). ¹H NMR (400 MHz, CDCl₃) δ 7.78 (d, J=1.6 Hz, 1H), 7.58 (d, J=7.6 Hz, 1H), 7.53 (dd, J=8.0 and 1.6 Hz, 1H), 7.44 (d, J=2.4 Hz, 1H), 7.26-7.25 (m, 1H), 6.27 (s, J=1H), 6.14-5.39 (m, 3H), 4.87-4.34 (m, 4H), 4.16 (d, J=12.8 Hz, 1H), 3.64 (dd, J=13.6 and 5.6 Hz, 1H), 3.07 (br s, 1H), 2.80 (d, J=4.8 Hz, 3H), 2.73 (d, J=16.0 Hz, 1H), 1.57-1.55 (m, 3H), 1.31 (d, J=6.4 Hz, 3H). ¹⁹F NMR (376 MHz, CDCl₃) δ −62.83, −71.76.

Compounds 15A and 15B (Preparation Method A)

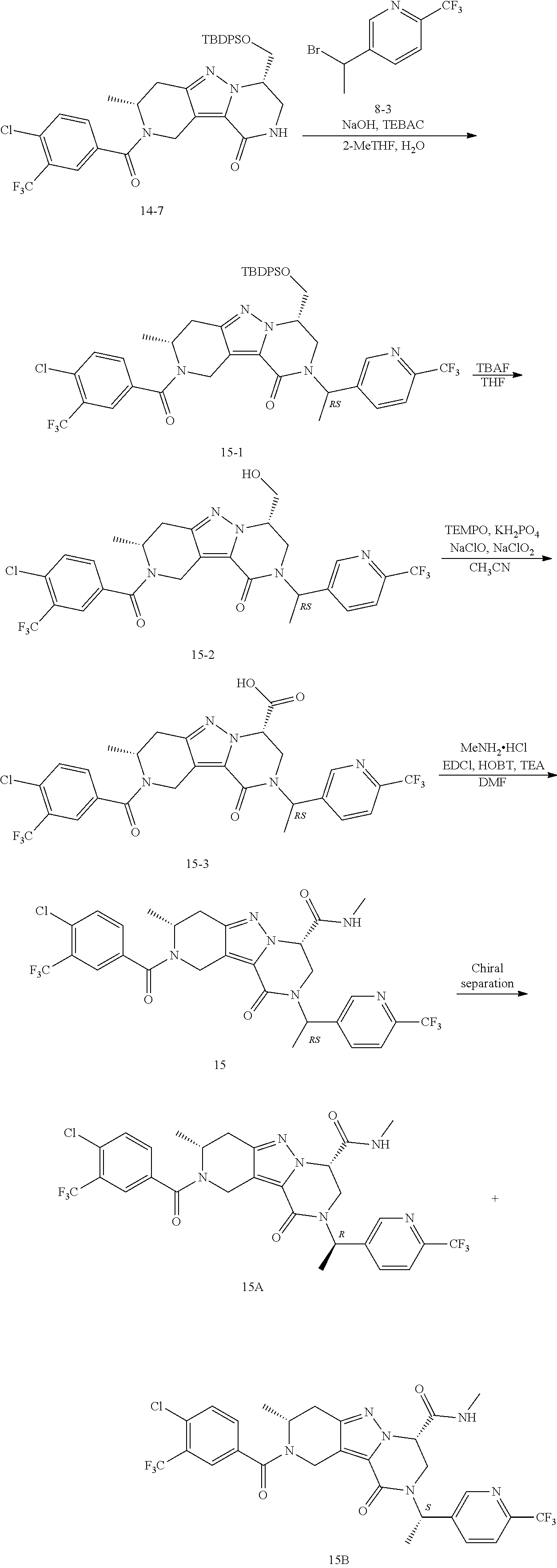

Intermediate 15-1

(3R,7S)-7-(((tert-Butyldiphenylsilyl)oxy)methyl)-2-(4-chloro-3-(trifluoromethyl)benzoyl)-3-methyl-9-(1-(6-(trifluoromethyl)pyridin-3-yl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(4-chloro-3-(trifluoromethyl)benzoyl)-3-methyl-1,2,3,4,8,9-hexahydropyrido[4′,3′:3,4]pyrazolo[1,5-a]pyrazin-10(7H)-one 14-7 (500 mg, 100% purity, 0.73 mmol) and 5-(1-bromoethyl)-2-(trifluoromethyl)pyridine 8-3 (370 mg, 100% purity, 1.46 mmol) in 2-methyltetrahydrofuran (2.5 mL) was added 50% wt. sodium hydroxide in water (2.5 mL) and N-benzyl-N,N-diethylethanaminium chloride (25 mg, 0.11 mmol) slowly at room temperature. After being stirred at room temperature for 3 hours, the mixture was diluted with water (5 mL) and concentrated at room temperature under reduced pressure to remove the volatile. The remained aqueous layer was acidified with 1 M hydrochloride aqueous solution (30 mL) and extracted with ethyl acetate (50 mL) twice. The combined organic layers were washed with brine (30 mL), dried over Na₂SO₄₍ₛ₎ and filtered. The filtrate was concentrated to give the crude compound (760 mg, 81% purity from LCMS, 98.2% yield) as yellow solids. LC-MS (ESI): R_{T}=2.31 min, mass calcd. for C₄₃H₄₂ClF₆N₅O₃Si 853.3, m/z found 854.2 [M+H]⁺.

Intermediate 15-2

(3R,7S)-2-(4-Chloro-3-(trifluoromethyl)benzoyl)-7-(hydroxymethyl)-3-methyl-9-(1-(6-(trifluoromethyl)pyridin-3-yl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(4-chloro-3-(trifluoromethyl)benzoyl)-3-methyl-9-(1-(6-(trifluoromethyl)pyridin-3-yl)ethyl)-1,2,3,4,8,9-hexahydropyrido[4′,3′:3,4]pyrazolo[1,5-a]pyrazin-10(7H)-one 15-1 (760 mg, 81% purity, 0.72 mmol) in tetrahydrofuran (10 mL) was added 1 M tetrabutylammonium fluoride in tetrahydrofuran (1 mL, 1 mmol) at 0° C. After being stirred at 0° C. for 1 hour, the mixture was concentrated under reduced pressure to give crude. The crude was purified by silica gel column chromatography (dichloromethane:methanol=10:1) to give the title compound (450 mg, 96% purity from LCMS, 97.3% yield) as yellow solids. LC-MS (ESI): R_{T}=1.62 min, mass calcd. for C₂₇H₂₄ClF₆N₅O₃ 615.2, m/z found 616.0 [M+H]⁺.

Intermediate 15-3

(3R,7S)-2-(4-Chloro-3-(trifluoromethyl)benzoyl)-3-methyl-10-oxo-9-(1-(6-(trifluoromethyl)pyridin-3-yl)ethyl)-1,2,3,4,7,8,9,10-octahydropyrido[4′,3′:3,4]pyrazolo[1,5-a]pyrazine-7-carboxylic acid

To a solution of (3R,7S)-2-(4-chloro-3-(trifluoromethyl)benzoyl)-7-(hydroxymethyl)-3-methyl-9-(1-(6-(trifluoromethyl)pyridin-3-yl)ethyl)-1,2,3,4,8,9-hexahydropyrido[4′,3′:3,4]pyrazolo[1,5-a]pyrazin-10(7H)-one 15-2 (450 mg, 96% purity, 0.70 mmol) in acetonitrile (3 mL) was added 2,2,6,6-Tetramethylpiperidinooxy (230 mg, 1.47 mmol), sodium chlorite (160 mg, 80% purity, 1.42 mmol), saturated potassium dihydrogenphosphate aqueous solution (3 mL) and sodium hypochlorite (0.9 mL, 1.51 mmol) at 0° C. After being stirred at 0° C. overnight, the mixture was diluted with sodium sulfite saturated solution (20 mL) and extracted with ethyl acetate (50 mL) twice. The combined organic layers were washed with brine (30 mL), dried over Na₂SO₄₍ₛ₎ and filtered. The filtrate was concentrated and purified by C18 column (acetonitrile:water=5% to 100%) to give the title compound (400 mg, 100% purity from LCMS, 90.5% yield) as yellow solids. LC-MS (ESI): R_{T}=1.34 min, mass calcd. for C₂₇H₂₂ClF₆N₅O₄ 629.1, m/z found 630.0 [M+H]⁺.

Compound 15

(3R,7S)-2-(4-Chloro-3-(trifluoromethyl)benzoyl)-N,3-dimethyl-10-oxo-9-(1-(6-(trifluoromethyl)pyridin-3-yl)ethyl)-1,2,3,4,7,8,9,10-octahydropyrido[4′,3′:3,4]pyrazolo[1,5-a]pyrazine-7-carboxamide

To the solution of (3R,7S)-2-(4-chloro-3-(trifluoromethyl)benzoyl)-3-methyl-10-oxo-9-(1-(6-(trifluoromethyl)pyridin-3-yl)ethyl)-1,2,3,4,7,8,9,10-octahydropyrido[4′,3′:3,4]pyrazolo[1,5-a]pyrazine-7-carboxylic acid 15-3 (340 mg, 100% purity, 0.54 mmol) in N,N-dimethylformamide (5 mL) was added methanamine hydrochloride (80 mg, 1.19 mmol), 1-hydroxybenzotriazole (150 mg, 1.11 mmol), N1-((ethylimino)methylene)-N3,N3-dimethylpropane-1,3-diamine hydrochloride (220 mg, 1.15 mmol) and triethylamine (0.6 mL, 4.31 mmol) at 0° C. The mixture was stirred at room temperature for 16 hours. The mixture was diluted with water (20 mL), acidized with 0.5 M hydrochloric acid aqueous solution to pH˜5 and extracted with ethyl acetate (50 mL) twice. The combined organic layers were washed with brine (20 mL), dried over Na₂SO₄₍ₛ₎ and filtered. The filtrate was concentrated and purified by C18 column (acetonitrile:water=5% to 100%) to give the title compound P1 (175 mg, 100% purity from LCMS, 50.4% yield) as yellow solids. LC-MS (ESI): R_{T}=1.61 min, mass calcd. for C₂₈H₂₅ClF₆N₆O₃ 642.2, m/z found 643.1 [M+H]⁺.

Compounds 15A and 15B

(3R,7S)-2-(4-Chloro-3-(trifluoromethyl)benzoyl)-N,3-dimethyl-10-oxo-9-((R)-1-(6-(trifluoromethyl)pyridin-3-yl)ethyl)-1,2,3,4,7,8,9,10-octahydropyrido[4′,3′:3,4]pyrazolo[1,5-a]pyrazine-7-carboxamide (15A), and (3R,7S)-2-(4-chloro-3-(trifluoromethyl)benzoyl)-N,3-dimethyl-10-oxo-9-((S)-1-(6-(trifluoromethyl)pyridin-3-yl)ethyl)-1,2,3,4,7,8,9,10-octahydropyrido[4′,3′:3,4]pyrazolo[1,5-a]pyrazine-7-carboxamide (15B)

The racemate of (3R,7S)-2-(4-chloro-3-(trifluoromethyl)benzoyl)-N,3-dimethyl-10-oxo-9-(1-(6-(trifluoromethyl)pyridin-3-yl)ethyl)-1,2,3,4,7,8,9,10-octahydropyrido[4′,3′:3,4]pyrazolo[1,5-a]pyrazine-7-carboxamide compound 15 (175 mg, 100% purity, 0.27 mmol) was separated by chiral Prep. HPLC (separation condition: Column: Chiralpak IE 5 μm 30*250 mm, Mobile Phase: Hex:EtOH=30:70 at 30 mL/min; Temp: 30° C.; Wavelength: 254 nm) to afford the crude isomers, which were further purified by C18 column (acetonitrile:water=5% to 100%) to give compound 15A (30 mg, 98.2% purity, 16.8% yield, 100% stereopure) as white solids and compound 15B (68 mg, 98.5% purity, 38.3% yield, 99.6% stereopure) as white solids.

Compound 15A

LC-MS (ESI): R_{T}=3.962 min, mass calcd. for C₂₈H₂₅ClF₆N₆O₃ 642.2, m/z found 643.3 [M+H]⁺. Chiral HPLC (Column: Chiralpak IE 5 μm 4.6*250 mm; Mobile Phase: Hex:EtOH=30:70 at 1 mL/min; Temp: 30° C.; Wavelength: 254 nm, R_{T}=5.031 min). ¹H NMR (400 MHz, CDCl₃) δ 8.66 (s, 1H), 7.83 (d, J=7.6 Hz, 1H), 7.78 (d, J=1.2 Hz, 1H), 7.67 (d, J=8.4 Hz, 1H), 7.61-7.53 (m, 2H), 7.26-7.25 (m, 1H), 5.96-5.46 (m, 3H), 4.90-4.31 (m, 2H), 4.03-3.89 (m, 2H), 3.06 (br s, 1H), 2.75-2.67 (m, 4H), 1.67 (d, J=6.8 Hz, 3H), 1.32 (d, J=6.8 Hz, 3H). ¹⁹F NMR (376 MHz, CDCl₃) δ −62.85, −67.91.

Compound 15B

LC-MS (ESI): R_{T}=4.042 min, mass calcd. for C₂₈H₂₅ClF₆N₆O₃ 642.2, m/z found 643.4 [M+H]⁺. Chiral HPLC (Column: Chiralpak IE 5 μm 4.6*250 mm, Mobile Phase: Hex:EtOH=30:70 at 1 mL/min; Temp: 30° C.; Wavelength: 254 nm, R_{T}=6.442 min). ¹H NMR (400 MHz, CDCl₃) δ 8.74 (s, 1H), 7.87 (d, J=6.8 Hz, 1H), 7.78 (s, 1H), 7.68 (d, J=8.4 Hz, 1H), 7.61-7.53 (m, 2H), 7.26-7.25 (m, 1H), 6.01-5.45 (m, 3H), 4.88-4.46 (m, 2H), 4.20 (d, J=11.6 Hz, 1H), 3.48 (dd, J=4.4, 12.8 Hz, 1H), 3.05 (br s, 1H), 2.82-2.71 (m, 4H), 1.69 (d, J=7.2 Hz, 3H), 1.31 (d, J=6.4 Hz, 3H). ¹⁹F NMR (376 MHz, CDCl₃) δ −62.84, −67.97.

Compound 15B (Preparation Method B)

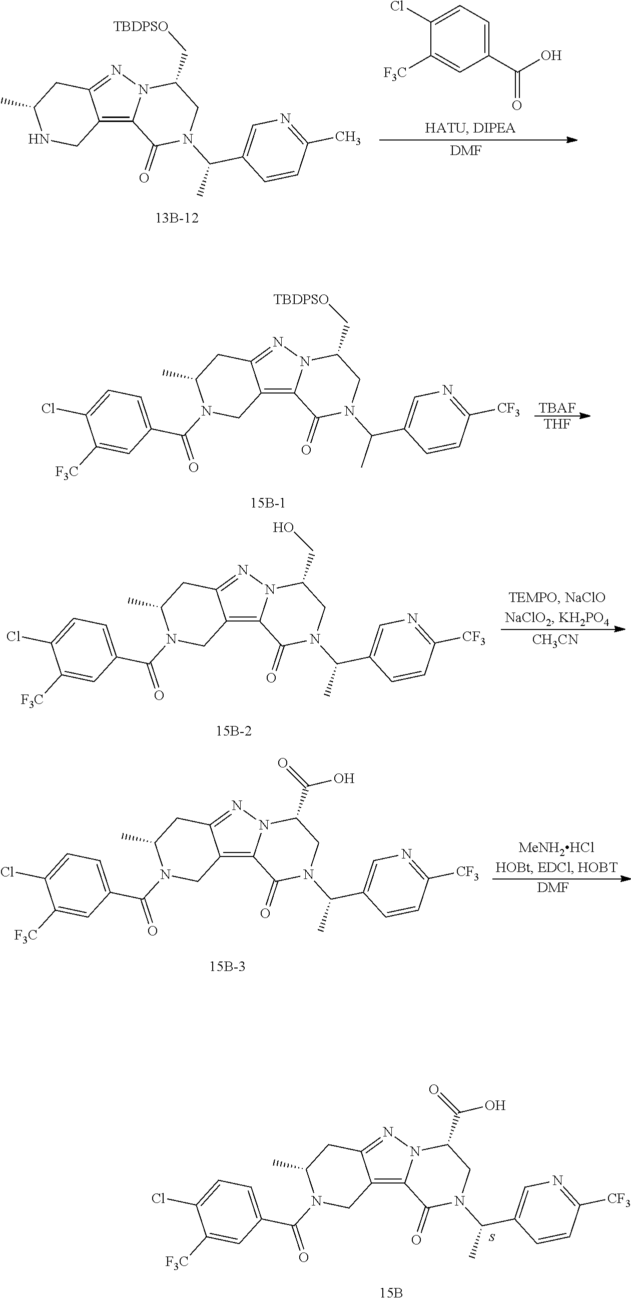

Intermediate 15B-1

(3R,7S)-7-(((tert-Butyldiphenylsilyl)oxy)methyl)-2-(4-chloro-3-(trifluoromethyl)benzoyl)-3-methyl-9-((S)-1-(6-(trifluoromethyl)pyridin-3-yl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-3-methyl-9-((S)-1-(6-(trifluoromethyl)pyridin-3-yl)ethyl)-1,2,3,4,8,9-hexahydropyrido[4′,3′:3,4]pyrazolo[1,5-a]pyrazin-10(7H)-one 13B-12 (10.8 g, 95% purity, 15.8 mmol) and 4-chloro-3-(trifluoromethyl)benzoic acid (5.30 g, 23.6 mmol) in N,N-dimethylformamide (100 mL) were added 2-(7-azabenzotriazol-1-yl)-N,N,N′,N′-tetramethyluronium hexafluorophosphate (12.2 g, 32.1 mmol) and N-ethyl-N-isopropylpropan-2-amine (13 mL, 76.7 mmol) at 0° C. under nitrogen atmosphere. After stirred overnight at room temperature, the reaction was quenched with water (100 mL) and extracted with ethyl acetate (100 mL) for three times. The organic layers were washed with brine (50 mL), dried over Na₂SO₄₍ₛ₎ and filtered. The filtrate was concentrated under reduced pressure. The residue was purified by silica gel column chromatography (petroleum ether:ethyl acetate=3:1 to 1:1) to give the compound (13.6 g, 95% purity from ¹H NMR, 99% yield) as white solids. LC-MS (ESI): R_{T}=2.15 min, mass calcd. for C₄₃H₄₂ClF₆N₅O₃Si 853.3, m/z found 854.3[M+H]⁺. ¹H NMR (400 MHz, CDCl₃) δ 8.72 (s, 1H), 7.87-7.76 (m, 2H), 7.69 (d, J=8.0 Hz, 1H), 7.60-7.57 (m, 5H), 7.53-7.37 (m, 7H), 6.18-6.01 (m, 1H), 5.76-5.26 (m, 1H), 4.80-4.35 (m, 3H), 4.09-4.08 (m, 1H), 3.84 (t, J=9.6 Hz, 1H), 3.73-3.70 (m, 1H), 3.39 (dd, J=13.2 and 4.4 Hz, 1H), 3.09-2.91 (m, 1H), 2.63-2.59 (m, 1H), 1.62 (s, 3H), 1.60 (s, 3H), 1.03 (s, 9H).

Intermediate 15B-2

(3R,7S)-2-(4-Chloro-3-(trifluoromethyl)benzoyl)-7-(hydroxymethyl)-3-methyl-9-((S)-1-(6-(trifluoromethyl)pyridin-3-yl)ethyl)-1,2,3,4,8,9-hexahydropyrido[4′,3′:3,4]pyrazolo[1,5-a]pyrazin-10(7H)-one

To a solution of (3R,7S)-7-(((tert-Butyldiphenylsilyl)oxy)methyl)-2-(4-chloro-3-(trifluoromethyl)benzoyl)-3-methyl-9-((S)-1-(6-(trifluoromethyl)pyridin-3-yl)ethyl)-1,2,3,4,8,9-hexahydropyrido[4′,3′:3,4]pyrazolo[1,5-a]pyrazin-10(7H)-one 15B-1 (13.6 g, 95% purity, 15.1 mmol) in tetrahydrofuran (135 mL) was added 1 M tetrabutylammonium fluoride in tetrahydrofuran (23.5 mL, 23.5 mmol) at 0° C. After stirred at 0° C. for 1 hour, the reaction mixture was quenched with water (50 mL) and extracted with ethyl acetate (50 mL) twice. The combined organic layers were washed with brine (50 mL) twice, dried over Na₂SO₄₍ₛ₎ and filtered. The filtrate was concentrated and purified by column chromatography on silica gel (petroleum ether:ethyl acetate=3:1 to 0:1) to give compound and the residue was recrystallized from (petroleum ether/ethyl acetate)=(v/v=75/50 mL) to afford title compound (7.0 g, 95% purity from LCMS, 71% yield) as white solids. LC-MS (ESI): R_{T}=3.023 min, mass calcd. for C₂₇H₂₄ClF₆N₅O₃ 615.2, m/z found 616.2 [M+H]⁺.

Intermediate 15B-3

(3R,7S)-2-(4-Chloro-3-(trifluoromethyl)benzoyl)-3-methyl-10-oxo-9-((S)-1-(6-(trifluoromethyl)pyridin-3-yl)ethyl)-1,2,3,4,7,8,9,10-octahydropyrido[4′,3′:3,4]pyrazolo[1,5-a]pyrazine-7-carboxylic acid

To a solution of (3R,7S)-2-(4-chloro-3-(trifluoromethyl)benzoyl)-7-(hydroxymethyl)-3-methyl-9-((S)-1-(6-(trifluoromethyl)pyridin-3-yl)ethyl)-1,2,3,4,8,9-hexahydropyrido[4′,3′:3,4]pyrazolo[1,5-a]pyrazin-10(7H)-one 15B-2 (6.28 g, 95% purity, 9.69 mmol) in acetonitrile (95 mL) were added saturated potassium dihydrogenphosphate aqueous (95 mL), sodium chlorite (2.20 g, 80% purity, 19.5 mmol), 2,2,6,6-tetramethylpiperidinooxy (3.14 g, 20.1 mmol) and sodium hypochlorite (11.9 mL, 10% purity, 19.9 mmol). After stirred at 0° C. for 8 hours, the reaction mixture was filtered, washed with acetonitrile (30 mL), the reaction was quenched with saturated sodium sulfite aqueous solution (10 mL), acidized with 1 M hydrochloride aqueous solution to pH˜4, then extracted with ethyl acetate (50 mL) twice. The combined organic layers were washed with brine (50 mL), dried over Na₂SO₄₍ₛ₎, filtered. The filtrate was concentrated to give a residue, which was triturated with (petroleum ether/ethyl acetate)=(v/v=10/2, 30 mL). The resulting solids was filtered through a funnel, washed with (petroleum ether/ethyl acetate)=(v/v=60/12 mL), and collected to afford the title compound (6.20 g, 95% purity from LCMS, 96% yield) as white solids. LC-MS (ESI): R_{T}=1.42 min, mass calcd. for C₇H₂₂ClF₆N₅O₄ 629.1, m/z found 630.4 [M+H]⁺.

Compound 15B

(3R,7S)-2-(4-chloro-3-(trifluoromethyl)benzoyl)-N,3-dimethyl-10-oxo-9-((S)-1-(6-(trifluoromethyl)pyridin-3-yl)ethyl)-1,2,3,4,7,8,9,10-octahydropyrido[4′,3′:3,4]pyrazolo[1,5-a]pyrazine-7-carboxamide

To a solution of (3R,7S)-2-(4-chloro-3-(trifluoromethyl)benzoyl)-3-methyl-10-oxo-9-((S)-1-(6-(trifluoromethyl)pyridin-3-yl)ethyl)-1,2,3,4,7,8,9,10-octahydropyrido[4′,3′:3,4]pyrazolo[1,5-a]pyrazine-7-carboxylic acid 15B-3 (7.19 g, 95% purity, 11.4 mmol), methylamine hydrochloride (1.93 g, 28.6 mmol), 1-hydroxybenzotriazole (3.09 g, 22.9 mmol) and N1-((ethylimino)methylene)-N3,N3-dimethylpropane-1,3-diamine hydrochloride (4.38 g, 22.8 mmol) in N,N-dimethylformamide (200 mL) was added a solution of triethylamine (10.4 mL, 74.6 mmol) in N,N-dimethyl formamide (15 mL) dropwise over 2 hours at 0° C. The reaction mixture was diluted with water (200 mL) and extracted with ethyl ether (100 mL) for three times. The combined organic phase was acidified to pH=5 with 0.5 M hydrochloride aqueous solution and extracted with ethyl acetate (300 mL) twice. The combined organic layers were washed with water (100 mL) for three times and brine (100 mL), dried over Na₂SO₄₍ₛ₎ and filtered. The filtrate was concentrated under reduced pressure, purified by C18 column (acetonitrile:water=30% to 70%) to give the title compound (6.90 g, 98.6% purity from LCMS, 82% yield, 99.3% stereopure) as white solids. LC-MS (ESI): R_{T}=8.509 min, mass calcd. for C₂₈H₂₅ClF₆N₆O₃ 642.2, m/z found 643.1 [M+H]⁺. Chiral analysis (Column: Chiralpak IE 5 μm 4.6*250 mm; Mobile Phase: Hex:EtOH=30:70 at 1.0 mL/min; Temp: 30° C.; Wavelength: 254 nm, R_{T}=6.431 min). ¹H NMR (400 MHz, CDCl₃) δ 8.74 (s, 1H), 7.88-7.86 (m, 1H), 7.78 (d, J=1.6 Hz, 1H), 7.69 (d, J=8.4 Hz, 1H), 7.61-7.59 (m, 1H), 7.55-7.52 (m, 1H), 7.26-7.25 (m, 1H), 6.18-6.00 (m, 2H), 5.72-5.11 (m, 1H), 4.88 (s, 1H), 4.55-4.37 (m, 1H), 4.21 (d, J=11.2 Hz, 1H), 3.50 (dd, J=12.8, 4.8 Hz, 1H), 3.14-2.97 (m, 1H), 2.81 (d, J=4.8 Hz, 3H), 2.75-2.71 (m, 1H), 1.69 (d, J=7.2 Hz, 3H), 1.31 (d, J=6.8 Hz, 3H). ¹⁹F NMR (376 MHz, CDCl₃) δ −62.84, −67.97. Compounds 16A and 16B

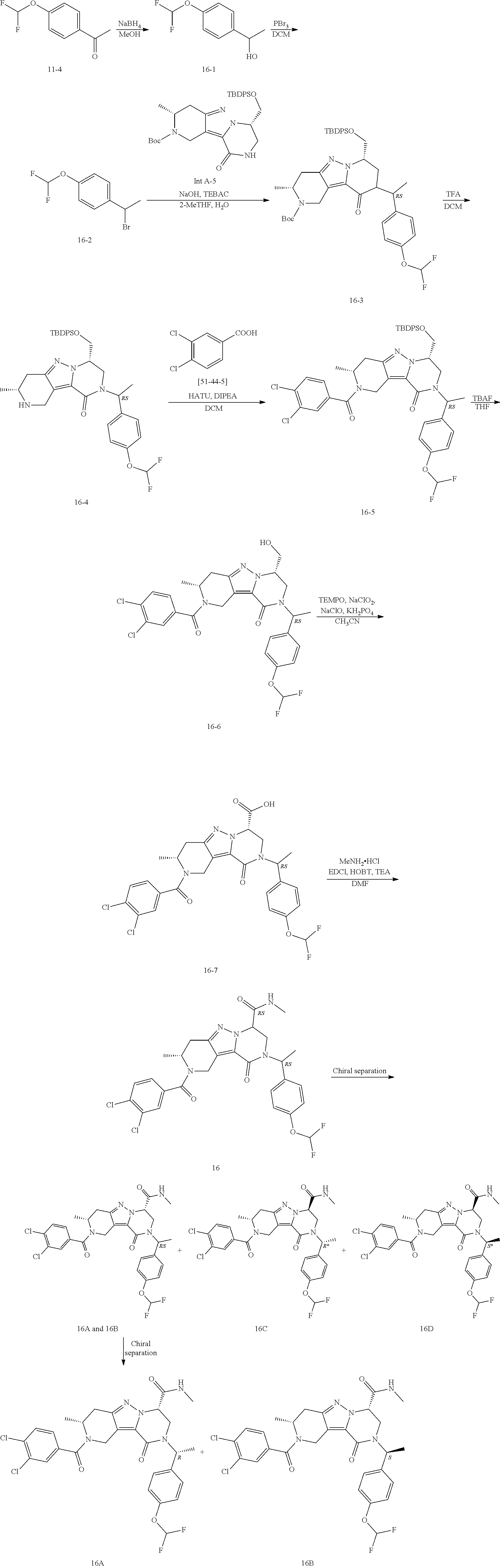

Intermediate 16-1

1-(4-(Difluoromethoxy)phenyl)ethanol

To a solution of 1-(4-(difluoromethoxy)phenyl)ethanone 11-4 (5.0 g, 26.9 mmol) in methanol (50 mL) was slowly added sodium borohydride (3.0 g, 79.3 mmol) at 0° C. After being stirred at room temperature for 3 hours, the reaction mixture was slowly quenched with acetone (50 mL) and concentrated to give a residue. The residue was dissolved into ethyl acetate (50 mL), washed with brine (50 mL) twice, dried over Na₂SO₄₍ₛ₎ and filtered. The filtrate was concentrated to give the title compound (5.3 g, 90% purity from ¹H NMR, 94% yield) as a colorless oil. ¹H NMR (400 MHz, CDCl₃) δ 7.38 (d, J=8.4 Hz, 2H), 7.10 (d, J=8.4 Hz, 1H), 6.50 (t, J=74.0 Hz, 1H), 4.91 (q, J=6.0 Hz, 1H), 1.80 (d, J=3.6 Hz, 1H), 1.49 (d, J=6.4 Hz, 3H).

Intermediate 16-2

1-(1-Bromoethyl)-4-(difluoromethoxy)benzene

To a solution of 1-(4-(difluoromethoxy)phenyl)ethanol 16-1 (500 mg, 90% purity, 2.39 mmol) in dichloromethane (5 mL) was slowly added phosphorus(III) bromide (650 mg, 2.40 mmol) at 0° C. After being stirred at room temperature for 2 hours, the reaction mixture was poured into ice water (20 mL) and extracted with dichloromethane (30 mL) twice. The combined organic layers were washed with saturated sodium bicarbonate solution (50 mL×2), brine (50 mL×2), dried over Na₂SO₄₍ₛ₎ and filtered. The filtrate was concentrated under reduced pressure to give the title compound (590 mg, 95% purity from ¹H NMR, 93% yield) as a colorless oil. ¹H NMR (400 MHz, CDCl₃) δ 7.44 (d, J=8.4 Hz, 2H), 7.09 (d, J=8.8 Hz, 2H), 6.51 (t, J=73.6 Hz, 1H), 5.20 (q, J=7.2 Hz, 1H), 2.03 (d, J=6.8 Hz, 3H).

Intermediate 16-3

(3R,7S)-tert-Butyl 7-(((tert-butyldiphenylsilyl)oxy)methyl)-9-(1-(4-(difluoromethoxy)phenyl)ethyl)-3-methyl-10-oxo-3,4,7,8,9,10-hexahydropyrido[4′,3′:3,4]pyrazolo[1,5-a]pyrazine-2(1H)-carboxylate

To a solution of (3R,7S)-tert-butyl 7-(((tert-butyldiphenylsilyl)oxy)methyl)-3-methyl-10-oxo-3,4,7,8,9,10-hexahydropyrido[4′,3′:3,4]pyrazolo[1,5-a]pyrazine-2(1H)-carboxylate Int A-5 (1 g, 100% purity, 1.74 mmol) in 2-methyltetrahydrofuran (4 mL) was added 1-(1-bromoethyl)-4-(difluoromethoxy)benzene 16-2 (1.4 g, 5.02 mmol), N-benzyl-N,N-diethylethanaminium chloride (198 mg, 0.869 mmol) and 50% wt. sodium hydroxide in water (4 mL) at 0° C. After being stirred at 30° C. for 2 hours, the mixture was diluted with water (8 mL), and extracted with ethyl acetate (10 mL) for three times. The combined organic layers were washed with brine (8 mL), dried over Na₂SO₄₍ₛ₎, and filtered. The filtrate was concentrated and purified by C18 column (acetonitrile:water=5% to 100%) to give the title compound (940 mg, 100% purity from LCMS, 73% yield) as white solids. LC-MS (ESI): R_{T}=2.27 min and 2.33 min, mass calcd. for C₄₁H₅₀F₂N₄O₅Si 744.4, m/z found 745.0 [M+H]⁺.

Intermediate 16-4

(3R,7S)-7-(((tert-Butyldiphenylsilyl)oxy)methyl)-9-(1-(4-(difluoromethoxy)phenyl)ethyl)-3-methyl-1,2,3,4,8,9-hexahydropyrido[4′,3′:3,4]pyrazolo[1,5-a]pyrazin-10(7H)-one

To a solution of (3R,7S)-tert-butyl 7-(((tert-butyldiphenylsilyl)oxy)methyl)-9-(1-(4-(difluoromethoxy)phenyl)ethyl)-3-methyl-10-oxo-3,4,7,8,9,10-hexahydropyrido[4′,3′:3,4]pyrazolo[1,5-a]pyrazine-2(1H)-carboxylate 16-3 (940 mg, 100% purity, 1.26 mmol) in dichloromethane (4 mL) was added 2,2,2-trifluoroacetic acid (2 mL) at 0° C. After being stirred at 0° C. for 1 hour, the reaction mixture was quenched with saturated sodium bicarbonate aqueous solution (10 mL) to pH=10˜11, and extracted with dichloromethane (15 mL) for three times. The organic layers were combined, washed with brine (10 mL), dried over Na₂SO₄₍ₛ₎ and filtered. The filtrate was concentrated to give the title compound (800 mg, 100% purity from LCMS, 98% yield) as a colorless oil. LC-MS (ESI): R_{T}=0.77&0.82 min, mass calcd. for C₃₆H₄₂F₂N₄O₃Si 644.3, m/z found 645.5 [M+H]⁺.

Intermediate 16-5

(3R,7S)-7-(((tert-Butyldiphenylsilyl)oxy)methyl)-2-(3,4-dichlorobenzoyl)-9-(1-(4-(difluoromethoxy)phenyl)ethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-9-(1-(4-(difluoromethoxy)phenyl)ethyl)-3-methyl-1,2,3,4,8,9-hexahydropyrido[4′,3′:3,4]pyrazolo[1,5-a]pyrazin-10(7H)-one 16-4 (100 mg, 99% purity, 0.15 mmol), 3,4-dichlorobenzoic acid (40 mg, 0.21 mmol) and 2-(3H-[1,2,3]triazolo[4,5-b]pyridin-3-yl)-1,1,3,3-tetramethyl uronium hexafluorophosphate(V) (80 mg, 0.21 mmol) in N,N-dimethylformamide (1 mL), and N-ethyl-N-isopropylpropan-2-amine (70 mg, 0.54 mmol) was added at room temperature. Then the reaction mixture was stirred at room temperature under nitrogen atmosphere for 2 hours. The reaction mixture was concentrated and purified by C18 column (acetonitrile:water=5% to 90%) to give the title compound (130 mg, 90% purity from ¹H NMR, 93% yield) as white solids. LC-MS (ESI): R_{T}=1.97 and 2.04 min, mass calcd. for C₄₃H₄₄Cl₂F₂N₄O₄Si 816.24, m/z found 816.8 [M+H]⁺. ¹H NMR (400 MHz, CDCl₃) δ 7.60-7.49 (m, 5H), 7.46-7.29 (m, 10H), 7.11 (d, J=8.0 Hz, 1H), 6.94 (d, J=8.4 Hz, 1H), 6.50 (t, J=73.6 Hz, 1H), 6.12-5.92 (m, 1H), 5.70-5.39 (m, 1H), 4.78-4.31 (m, 3H), 4.11-4.01 (m, 1H), 3.82-3.73 (m, 1H), 3.63-3.53 (m, 1H), 3.42-3.32 (m, 1H), 3.02-2.60 (m, 2H), 1.56-1.52 (m, 3H), 1.24-1.12 (m, 3H), 1.03 (s, 6H), 0.94 (s, 3H).

Intermediate 16-6

(3R,7S)-2-(3,4-Dichlorobenzoyl)-9-(1-(4-(difluoromethoxy)phenyl)ethyl)-7-(hydroxymethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-9-(1-(4-(difluoromethoxy)phenyl)ethyl)-3-methyl-1,2,3,4,8,9-hexahydropyrido[4′,3′:3,4]pyrazolo[1,5-a]pyrazin-10(7H)-one 16-5 (1 g, 90% purity, 1.10 mmol) in tetrahydrofuran (10 mL) was added 1 M tetrabutylammonium fluoride (0.75 mL, 0.75 mmol) at room temperature, then the mixture was stirred at room temperature for 1 hour. The reaction mixture was poured into water (10 mL) and extracted with ethyl acetate (20 mL) for three times. The combined organic layers were dried over Na₂SO₄₍ₛ₎ and filtered. The filtrate was concentrated under reduced pressure to give the residue, which was purified by column chromatography with silica gel (dichloromethane:methanol=50:1) to give the title compound (670 mg, 95% purity from LCMS, 100% yield) as yellow solids. LC-MS (ESI): R_{T}=1.721 min, mass calcd. for C₂₇H₂₆Cl₂F₂N₄O₄ 578.13, m/z found 578.9 [M+H]⁺.

Intermediate 16-7

(3R,7S)-2-(3,4-Dichlorobenzoyl)-9-(1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-9-(1-(4-(difluoromethoxy)phenyl)ethyl)-7-(hydroxymethyl)-3-methyl-1,2,3,4,8,9-hexahydropyrido[4′,3′:3,4]pyrazolo[1,5-a]pyrazin-10(7H)-one 16-6 (555 mg, 95% purity, 0.91 mmol) in acetonitrile (4 mL) was added saturated potassium dihydrogenphosphate aqueous solution (4 mL), 2,2,6,6-tetramethylpiperidinooxy (285 mg, 1.82 mmol), sodium chlorite (210 mg, 1.86 mmol), 5.5% sodium hypochlorite solution (1.1 mL, 1.85 mmol) at 0° C. The reaction mixture was allowed to slowly return to room temperature. After being stirred at room temperature for 8 hours, the reaction mixture was quenched with saturated sodium thiosulfate aqueous solution (5 mL), acidized with 1 M hydrochloric acid solution to pH=4˜5, and extracted with ethyl acetate (15 mL) for three times. The combined organic layers were washed with brine (5 mL), dried over Na₂SO₄₍ₛ₎, and filtered. The filtrate was concentrated and purified by C18 column (acetonitrile:water=5% to 100%) to give the title compound (480 mg, 100% purity from LCMS, 89% yield) as white solids. LC-MS (ESI): R_{T}=1.35 min, mass calcd. for C₂₇H₂₄Cl₂F₂N₄O₅ 592.1, m/z found 592.7 [M+H]⁺.

Compound 16

(3R)-2-(3,4-dichlorobenzoyl)-9-(1-(4-(difluoromethoxy)phenyl)ethyl)-N,3-dimethyl-10-oxo-1,2,3,4,7,8,9,10-octahydropyrido[4′,3′:3,4]pyrazolo[1,5-a]pyrazine-7-carboxamide

To a solution of (3R,7S)-2-(3,4-dichlorobenzoyl)-9-(1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-7-carboxylic acid 16-7 (400 mg, 100% purity, 0.67 mmol), methanamine hydrochloride (148 mg, 2.19 mmol), N1-((ethylimino)methylene)-N3,N3-dimethyl propane-1,3-diamine hydrochloride (246 mg, 1.28 mmol) and 1H-benzo[d][1,2,3]triazol-1-ol (246 mg, 1.82 mmol) in N,N-dimethylformamide (7 mL) was added triethylamine (395 mg, 3.90 mmol) at 0° C. The reaction mixture was allowed to slowly return to room temperature. After being stirred at room temperature under nitrogen atmosphere for overnight, the reaction mixture was quenched with water (10 mL) and extracted with ethyl acetate (20 mL) for three times. The combined organic layers were dried over Na₂SO₄₍ₛ₎ and filtered. The filtrate was concentrated and purified by C18 column (acetonitrile:water=5% to 100%) to give the title compound (340 mg, 100% purity from LCMS, 83% yield) as white solids. LC-MS (ESI): R_{T}=1.437 min, mass calcd. for C₂₈H₂₇Cl₂F₂N₅O₄ 605.1, m/z found 606.2 [M+H]⁺.

Compounds 16A, 16B, 16C, and 16D

(3R,7S)-2-(3,4-Dichlorobenzoyl)-9-(1-(4-(difluoromethoxy)phenyl)ethyl)-N,3-dimethyl-10-oxo-1,2,3,4,7,8,9,10-octahydropyrido[4′,3′:3,4]pyrazolo[1,5-a]pyrazine-7-carboxamide (16A and 16B), (3R,7R)-2-(3,4-dichlorobenzoyl)-9-((R*)-1-(4-(difluoromethoxy)phenyl)ethyl)-N,3-dimethyl-10-oxo-1,2,3,4,7,8,9,10-octahydropyrido[4′,3′:3,4]pyrazolo[1,5-a]pyrazine-7-carboxamide (16C), and (3R,7R)-2-(3,4-dichlorobenzoyl)-9-((S*)-1-(4-(difluoromethoxy)phenyl)ethyl)-N,3-dimethyl-10-oxo-1,2,3,4,7,8,9,10-octahydropyrido[4′,3′:3,4]pyrazolo[1,5-a]pyrazine-7-carboxamide (16D)

A mixture of (3R,7S)-2-(3,4-dichlorobenzoyl)-9-(1-(4-(difluoromethoxy)phenyl)ethyl)-N,3-dimethyl-10-oxo-1,2,3,4,7,8,9,10-octahydropyrido[4′,3′:3,4]pyrazolo[1,5-a]pyrazine-7-carboxamide compound 16 (440 mg, 96% purity, 0.70 mmol) was separated by chiral prep. HPLC (separation condition: Column: Chiralpak IE 5 μm 20*250 mm; Mobile Phase: MeOH:DCM=60:40 at 1 mL/min; Temp: 30° C.; Wavelength: 254 nm) to afford compound 16C (3 mg, 0.7% yield) as white solids and compound 16D (4 mg, 0.8% yield) as white solids and the title mixture compounds 16A and 16B (330 mg, 78% yield, 100% purity from LCMS) as white solids.

Compounds 16A and 16B

LC-MS (ESI): R_{T}=2.93 min, mass calcd. for C₂₈H₂₇Cl₂F₂N₅O₄ 605.14, m/z found 605.8 [M+H]⁺.

Compound 16C

LC-MS (ESI): R_{T}=1.65 min, mass calcd. for C₂₈H₂₇Cl₂F₂N₅O₄ 605.14, m/z found 622.8 [M+18]⁺. ¹H NMR (400 MHz, DMSO-d₆) δ 7.98 (s, 1H), 7.77-7.75 (m, 2H), 7.49-7.39 (m, 3H), 7.23-7.05 (m, 3H), 6.06-5.50 (m, 2H), 5.25-5.04 (m, 1H), 4.56-4.10 (m, 2H), 3.69-3.56 (m, 2H), 2.98-2.94 (m, 1H), 2.62 (s, 3H), 2.21-2.08 (m, 1H), 1.40 (s, 3H), 1.18-1.12 (m, 3H). ¹⁹F NMR (376 MHz, DMSO-d₆) δ −82.08.

Compound 16D

LC-MS (ESI): R_{T}=1.62 min, mass calcd. for C₂₈H₂₇Cl₂F₂N₅O₄ 605.14, m/z found 622.8 [M+18]⁺. ¹H NMR (400 MHz, CDCl₃) δ 7.56-7.52 (m, 2H), 7.30-7.28 (m, 4H), 7.09-7.07 (m, 2H), 6.50 (t, J=74.0 Hz, 1H), 5.91-5.44 (m, 3H), 4.86-4.36 (m, 2H), 3.91-3.79 (m, 2H), 3.08 (br s, 1H), 2.71-2.67 (m, 4H), 1.68-1.61 (m, 3H), 1.26-1.25 (m, 3H). ¹⁹F NMR (376 MHz, CDCl₃) δ −80.85.

Compounds 16A and 16B (Preparation Method A)

(3R,7S)-2-(3,4-dichlorobenzoyl)-9-((R)-1-(4-(difluoromethoxy)phenyl)ethyl)-N,3-dimethyl-10-oxo-1,2,3,4,7,8,9,10-octahydropyrido[4′,3′:3,4]pyrazolo[1,5-a]pyrazine-7-carboxamide (16A), and (3R,7S)-2-(3,4-dichlorobenzoyl)-9-((S)-1-(4-(difluoromethoxy)phenyl)ethyl)-N,3-dimethyl-10-oxo-1,2,3,4,7,8,9,10-octahydropyrido[4′,3′:3,4]pyrazolo[1,5-a]pyrazine-7-carboxamide (16B)

A mixture of (3R,7S)-2-(3,4-dichlorobenzoyl)-9-(1-(4-(difluoromethoxy)phenyl)ethyl)-N,3-dimethyl-10-oxo-1,2,3,4,7,8,9,10-octahydropyrido[4′,3′:3,4]pyrazolo[1,5-a]pyrazine-7-carboxamide compounds 16A and 16B (330 mg, 100% purity, 0.544 mmol) was separated by chiral prep. HPLC (separation condition: Column: Chiralpak IE 5 μm 20*250 mm; Mobile Phase: HEX:EtOH=40:60 at 1 mL/min; Temp: 30° C.; Wavelength: 254 nm) to afford compound 16A (66.8 mg, 20% yield, 98.5% purity, 100% stereopure) as white solids and compound 16B (90.7 mg, 27% yield, 99.4% purity, 99.9% stereopure) as white solids.

Compound 16A

LC-MS (ESI): R_{T}=3.366 min, mass calcd. for C₂₈H₂₇Cl₂F₂N₅O₄ 606, m/z found 607.2 [M+H]⁺. Chiral analysis (Column: Chiralpak IE 5 μm 4.6*250 mm; Mobile Phase: Hex:EtOH=40:60 at 1 mL/min; Col. Temp: 30° C.; Wavelength: 254 nm, Rt=8.184 min). ¹H NMR (400 MHz, CDCl₃) δ 7.54-7.51 (m, 2H), 7.31-7.27 (m, 4H), 7.09 (d, J=8.8 Hz, 2H), 6.51 (t, J=74.0 Hz, 1H), 6.07-5.44 (m, 3H), 4.86-4.42 (m, 2H), 3.92-3.81 (m, 2H), 3.06 (br s, 1H), 2.73-2.67 (m, 4H), 1.60-1.58 (m, 3H), 1.30 (d, J=6.4 Hz, 3H). ¹⁹F NMR (376 MHz, CDCl₃) δ −80.83.

Compound 16B

LC-MS (ESI): R_{T}=3.093 min, mass calcd. for C₂₈H₂₇Cl₂F₂N₅O₄ 606, m/z found 607.2 [M+H]⁺. Chiral analysis (Column: Chiralpak IE 5 μm 4.6*250 mm; Mobile Phase: Hex:EtOH=40:60 at 1 mL/min; Col. Temp: 30° C.; Wavelength: 254 nm, Rt=10.821 min). ¹H NMR (400 MHz, CDCl₃) δ 7.54-7.52 (m, 2H), 7.37-7.28 (m, 4H), 7.11 (d, J=8.4 Hz, 2H), 6.50 (t, J=73.6 Hz, 1H), 5.99-5.44 (m, 3H), 4.83-4.40 (m, 2H), 4.08-3.92 (m, 1H), 3.40-3.36 (m, 1H), 3.04 (br s, 1H), 2.80-2.69 (m, 4H), 1.58-1.56 (m, 3H), 1.30 (d, J=6.4 Hz, 3H). ¹⁹F NMR (376 MHz, CDCl₃) δ −81.03.

Compound 16B (Preparation Method B)

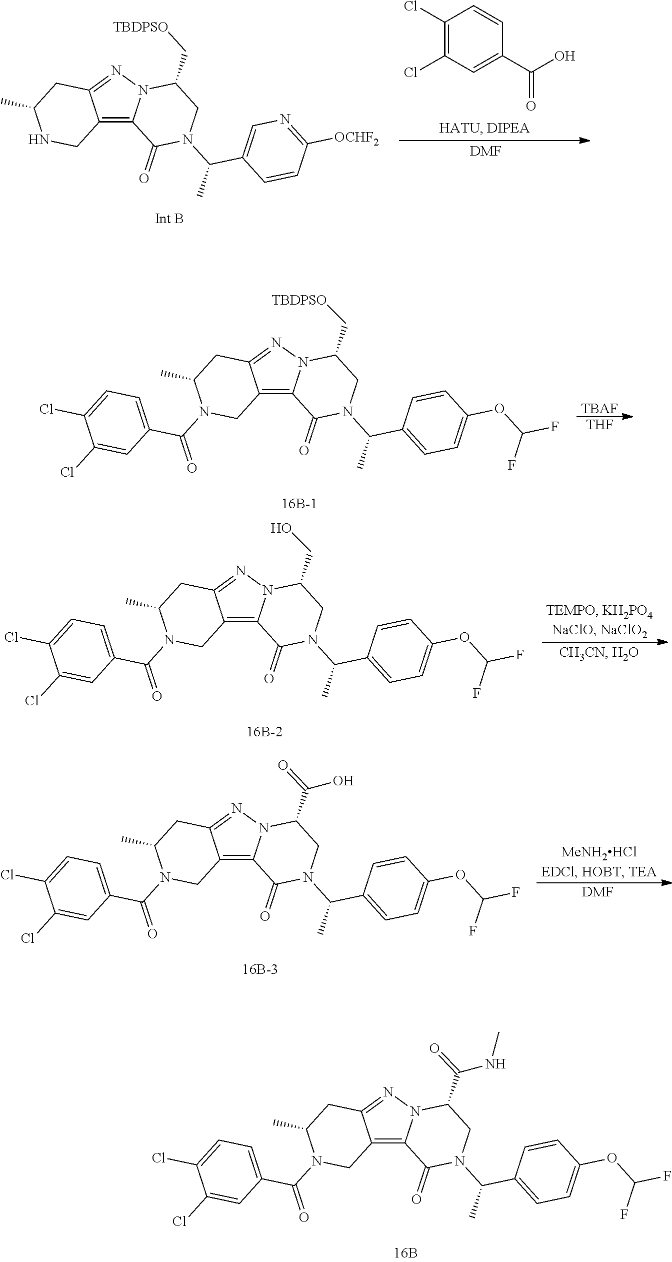

Intermediate 16B-1

(3R,7S)-7-(((tert-Butyldiphenylsilyl)oxy)methyl)-2-(3,4-dichlorobenzoyl)-9-((S)-1-(4-(difluoromethoxy)phenyl)ethyl)-3-methyl-1,2,3,4,8,9-hexahydropyrido [4′,3′:3,4]pyrazolo[1,5-a]pyrazin-10(7H)-one

To a solution of (3R,7S)-7-(((tert-butyldiphenylsilyl)oxy)methyl)-9-((S)-1-(4-(difluoro methoxy)phenyl)ethyl)-3-methyl-1,2,3,4,8,9-hexahydropyrido[4′,3′:3,4]pyrazolo[1,5-a]pyrazin-10(7H)-one Int B (10 g, 97% purity, 15.0 mmol) in N,N-dimethyl formamide (100 mL) were added 3,4-dichlorobenzoic acid (3.5 g, 18.3 mmol), N-ethyl-N-isopropylpropan-2-amine (9.5 mL, 51.5 mmol) and O-(7-azabenzotriazol-1-yl)-N,N, N′,N′-tetramethyluronium hexafluorophosphate (9.0 g, 23.7 mmol) at 0° C. After stirred at 30° C. for 1 hour, the mixture was concentrated and purified by C18 column (acetonitrile:water (+0.02% ammonium acetate)=5% to 100%) to give the title compound (8.0 g, 100% purity, 65% yield) as while solids. LC-MS (ESI): R_{T}=2.06 min, mass calcd. for C₄₃H₄₄Cl₂F₂N₄O₄Si 816.3, m/z found: no mass.

Intermediate 16B-2

(3R,7S)-2-(3,4-Dichlorobenzoyl)-9-((S)-1-(4-(difluoromethoxy)phenyl)ethyl)-7-(hydroxymethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-9-((S)-1-(4-(difluoromethoxy)phenyl)ethyl)-3-methyl-1,2,3,4,8,9-hexahydropyrido[4′,3′:3,4]pyrazolo[1,5-a]pyrazin-10(7H)-one 16B-1 (15.0 g, 100% purity, 18.3 mmol) in tetrahydrofuran (150 mL) was added 1 M tetrabutylammonium fluoride (25 mL, 25 mmol) in tetrahydrofuran at 0° C. After stirred at 0° C. for 4 hours, the mixture was filtered and concentrated under reduced pressure to give a residue, which was added ethyl acetate (20 mL) and petroleum ether (40 mL) at 30° C. and stirred at 30° C. for 1 hour. The reaction mixture was cooled to room temperature and filtered. The cake was collected by filtration to give the title compound (8.4 g, 100% purity, 79% yield) as white solids. LC-MS (ESI): R_{T}=1.63 min, mass calcd. for C₂₇H₂₆Cl₂F₂N₄O₄ 578.1, m/z found 579.2 [M+H]⁺.

Intermediate 16B-3

(3R,7S)-2-(3,4-dichlorobenzoyl)-9-((S)-1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-9-((S)-1-(4-(difluoromethoxy)phenyl) ethyl)-7-(hydroxymethyl)-3-methyl-1,2,3,4,8,9-hexahydropyrido[4′,3′:3,4]pyrazolo [1,5-a]pyrazin-10(7H)-one 16B-2 (4.0 g, 100% purity, 6.90 mmol) in acetonitrile (60 mL) were added 2,2,6,6-tetramethylpiperidinooxy (2.2 g, 14.1 mmol), sodium hypochlorite (8.4 mL, 14.1 mmol), sodium chlorite (1.6 g, 14.1 mmol), saturated sodium dihydrogenphosphate aqueous solution (60 mL) at 0° C. After stirred at 25° C. for 16 hours, the mixture was diluted with water (100 mL) and extracted with ethyl acetate (100 mL) twice. The combined organic layers were washed with brine (200 mL), dried over Na₂SO₄₍ₛ₎ and filtered. The filtrate was concentrated to give the title compound (4 g, 93% purity, 91% yield) as yellow solids. The crude 16B-3 (8.2 g, 93% purity, 12.9 mmol) was dissolved in ethyl acetate (20 mL) and petroleum ether (40 mL) at 30° C. After stirred at 30° C. for 1 hour, the reaction mixture was cooled to room temperature and filtered. The cake was collected by filtration to give the title compound (7.5 g, 100% purity, 98% yield) white solids. LC-MS (ESI): R_{T}=2.23 min, mass calcd. for C₂₇H₂₄Cl₂F₂N₄O₅ 592.1, m/z found 593.2 [M+H]⁺.

Compound 16B

(3R,7S)-2-(3,4-Dichlorobenzoyl)-9-((S)-1-(4-(difluoromethoxy)phenyl)ethyl)-N,3-dimethyl-10-oxo-1,2,3,4,7,8,9,10-octahydropyrido[4′,3′:3,4]pyrazolo[1,5-a]pyrazine-7-carboxamide

To a solution of (3R,7S)-2-(3,4-dichlorobenzoyl)-9-((S)-1-(4-(difluoromethoxy)phenyl) ethyl)-3-methyl-10-oxo-1,2,3,4,7,8,9,10-octahydropyrido[4′,3′:3,4]pyrazolo[1,5-a]pyrazine-7-carboxylic acid 16B-3 (4.0 g, 100% purity, 6.74 mmol) in N,N-dimethylformamide (120 mL) were added methylamine hydrochloride (1.2 g, 16.9 mmol), 1H-benzo[d][1,2,3]triazol-1-ol (1.85 g, 13.7 mmol), N1-((ethylimino)methylene)-N3,N3-dimethylpropane-1,3-diamine hydrochloride (2.6 g, 13.6 mmol) and triethylamine (6.4 mL, 44.3 mmol) at 0° C. After stirred at 0° C. for 2 hours, the mixture was added ethyl acetate (150 mL), washed with water (150 mL) twice and brine (200 mL), dried over Na₂SO₄₍ₛ₎ and filtered. The filtrate was concentrated under reduced pressure to give a crude (4.0 g, 98% yield, 100% purity) as yellow solids. The crude 16 (7.5 g, 100% purity, 12.4 mmol) was purified by C18 column (acetonitrile:water (+0.02% ammonium acetate)=5% to 100%) to give the title compound (5.5 g, 72.7% yield, 99.2% purity, ¹H NMR including 0.03% of acetonitrile) and (700 mg, 9.2% yield, 98.3% purity) as white solids. The crude (5.32 g, purity 99.2%, 8.77 mmol, including 0.03% acetonitrile) was further dried in vacuum drying oven at 40° C. for 2 hours, then cooled to room temperature to give the title compound (5.3 g, 99.2% purity, 98.8% yield, 99.6% stereopure) as white solids. LC-MS (ESI): R_{T}=8.800 min, mass calcd. for C₂₈H₂₇Cl₂F₂N₅O₄ 605.1, m/z found 606.1 [M+H]⁺. Chiral analysis (Column: Chiralpak IE 5 μm 4.6*250 mm; Mobile Phase: Hex:EtOH=40:60 at 1 mL/min; Col. Temp: 30° C.; Wavelength: 254 nm, 99.8% stereopure at Rt=10.736 min). Chiral analysis (Column: Chiralpak IE 5 μm 4.6*250 mm; Mobile Phase: MeOH:DCM=60:40 at 1 mL/min; Temp: 30° C.; Wavelength: 254 nm; 99.6% stereopure at Rt=3.984 min). ¹H NMR (400 MHz, CDCl₃) δ 7.54-7.52 (m, 2H), 7.37-7.35 (m, 2H), 7.28-7.26 (m, 1H), 7.12-7.09 (m, 2H), 6.50 (t, J=74.0 Hz, 1H), 6.00-5.42 (m, 3H), 4.82-4.36 (m, 3H), 4.09-4.05 (m, 1H), 3.37 (dd, J=13.2 and 4.8 Hz, 1H), 3.12-2.94 (m, 1H), 2.80-2.69 (m, 4H), 1.58-1.56 (m, 3H), 1.29 (d, J=6.8 Hz, 3H). ¹⁹F NMR (376 MHz, CDCl₃) δ −81.06.

Compound 17

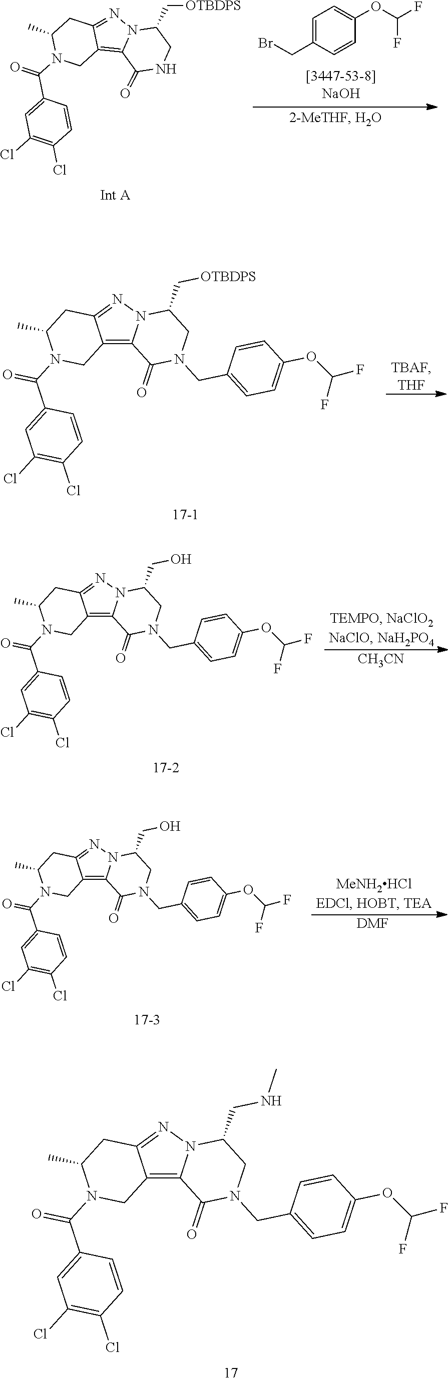

Intermediate 17-1

(3R,7S)-7-(((tert-Butyldiphenylsilyl)oxy)methyl)-2-(3,4-dichlorobenzoyl)-9-(4-(difluoromethoxy)benzyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (200 mg, 0.309 mmol, 100% purity) and 1-(bromomethyl)-4-(difluoromethoxy)benzene (130 mg, 0.548 mmol) in 2-methyltetrahydrofuran (5 mL) was added 50% wt. sodium hydroxide in water (5 mL) slowly at 30° C. After being stirred at 30° C. for 2 hours, the mixture was diluted with water (50 mL) and concentrated at room temperature under reduced pressure to remove the volatile. The remained aqueous layer was acidified with 2M hydrochloride aqueous solution (20 mL) and extracted with ethyl acetate (30 mL) twice and brine (60 mL), dried over Na₂SO₄₍ₛ₎ and filtered. The filtrate was concentrated to give the crude (300 mg, purity 51%, 60% yield) as a yellow oil. LC-MS (ESI): R_{T}=2.30 min, mass calcd. for C₄₂H₄₂Cl₂F₂N₄O₄Si 802.2, m/z found 803.1 [M+H]⁺.

Intermediate 17-2

(3R,7S)-2-(3,4-Dichlorobenzoyl)-9-(4-(difluoromethoxy)benzyl)-7-(hydroxymethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-9-(4-(difluoromethoxy)benzyl)-3-methyl-1,2,3,4,8,9-hexahydropyrido[4′,3′:3,4]pyrazolo[1,5-a]pyrazin-10(7H)-one 17-1 (450 mg, 51% purity, 0.286 mmol) in tetrahydrofuran (6 mL) was added 1 M tetrabutylammonium fluoride (1 mL, 1 mmol) in tetrahydrofuran at 0° C. After being stirred at 0° C. for 4 hours, the mixture was filtered and concentrated under reduced pressure to give crude. The crude was purified by C18 column (acetonitrile:water=5% to 100%) to give the title compound (170 mg, 98% yield, 93% purity) as a yellow oil. LC-MS (ESI): R_{T}=1.62 min, mass calcd. for C₂₆H₂₄Cl₂F₂N₄O₄ 564.1, m/z found 565.1 [M+H]⁺.

Intermediate 17-3

(3R,7S)-2-(3,4-Dichlorobenzoyl)-9-(4-(difluoromethoxy)benzyl)-3-methyl-10-oxo-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-9-(4-(difluoromethoxy)benzyl)-7-(hydroxymethyl)-3-methyl-1,2,3,4,8,9-hexahydropyrido[4′,3′:3,4]pyrazolo[1,5-a]pyrazin-10(7H)-one 17-2 (170 mg, 93% purity, 0.280 mmol) in acetonitrile (4 mL) was added 2,2,6,6-tetramethylpiperidinooxy (90 mg, 0.576 mmol), sodium hypochlorite (0.35 mL, 0.588 mmol), sodium chlorite (70 mg, 0.619 mmol), saturated sodium dihydrogenphosphate aqueous solution (4 mL) at 0° C. After being stirred at 0° C. for 1 hour, the mixture was diluted with water (20 mL) and extracted with ethyl acetate (20 mL) twice. The combined organic layers were washed with brine (40 mL), dried over Na₂SO₄₍ₛ₎ and filtered. The filtrate was concentrated to give the residue, which was dissolved in acetonitrile (4 mL). After being stirred at 40° C. for 1 hour, the mixture was cooled to room temperature and filtered. The white solids were collected by filtration to give the title compound (160 mg, 100% purity, 99% yield). LC-MS (ESI): R_{T}=1.32 min, mass calcd. for C₂₆H₂₂Cl₂F₂N₄O₅ 578.1, m/z found 579.0 [M+H]⁺.

Compound 17

(3R,7S)-2-(3,4-Dichlorobenzoyl)-9-(4-(difluoromethoxy)benzyl)-N,3-dimethyl-10-oxo-1,2,3,4,7,8,9,10-octahydropyrido[4′,3′:3,4]pyrazolo[1,5-a]pyrazine-7-carboxamide

To a solution of (3R,7S)-2-(3,4-dichlorobenzoyl)-9-(4-(difluoromethoxy)benzyl)-3-methyl-10-oxo-1,2,3,4,7,8,9,10-octahydropyrido[4′,3′:3,4]pyrazolo[1,5-a]pyrazine-7-carboxylic acid 17-3 (160 mg, 0.276 mmol, purity 100%) in N,N-dimethylformamide (4 mL) was added methylamine hydrochloride (55 mg, 0.774 mmol), 1H-benzo[d][1,2,3]triazol-1-ol (70 mg, 0.518 mmol), N1-((ethylimino)methylene)-N3,N3-dimethylpropane-1,3-diamine hydrochloride (110 mg, 0.574 mmol) and triethylamine (0.3 mL, 2.08 mmol) at 0° C. After being stirred at 0° C. for 2 hours, the mixture was added ethyl acetate (50 mL), washed with water (50 mL) twice, brine (50 mL), dried over Na₂SO₄₍ₛ₎ and filtered. The filtrate was concentrated under reduced pressure to give crude. The crude was purified by C18 column (acetonitrile:water=5% to 100%) to give the title compound (70 mg, 42.4% yield, 99.1% purity) as a yellow oil. LC-MS (ESI): R_{T}=3.292 min, mass calcd. for C₂₇H₂₅Cl₂F₂N₅O₄ 591.1, m/z found 592.0 [M+H]⁺. ¹H NMR (400 MHz, CDCl₃) δ 7.53-7.51 (m, 2H), 7.32-7.30 (m, 2H), 7.27-7.26 (m, 1H), 7.25-7.24 (m, 1H), 7.09 (d, J=8.4 Hz, 2H), 6.49 (t, J=73.6 Hz, 1H), 6.14-6.32 (m, 2H), 4.90-4.42 (m, 4H), 4.19-4.15 (m, 1H), 3.87 (dd, J=13.2 and 4.0 Hz, 1H), 3.13-2.97 (m, 1H), 2.78-2.70 (m, 4H), 1.29 (d, J=6.8 Hz, 3H). ¹⁹F NMR (376 MHz, CDCl₃) δ −80.95.

Compounds 18A and 18B

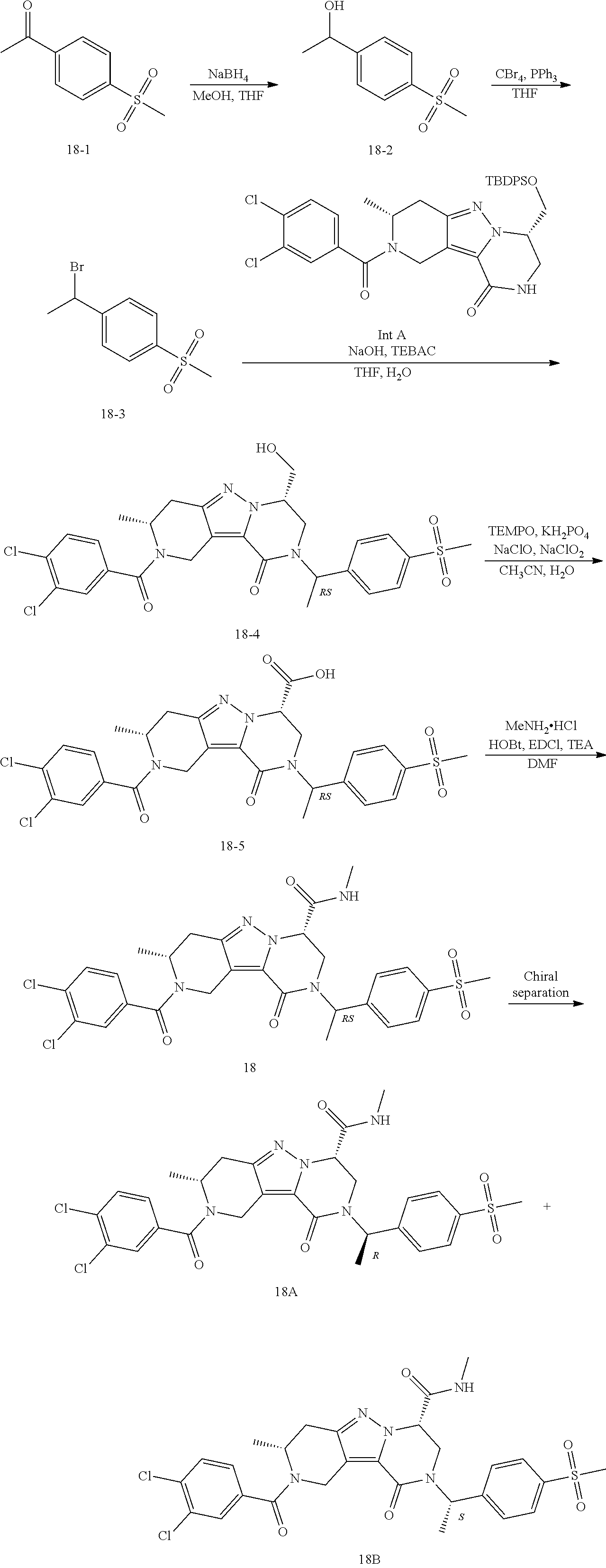

Intermediate 18-2

1-(4-(Methylsulfonyl)phenyl)ethan-1-ol

1-(4-(methylsulfonyl)phenyl)ethenone 18-1 (2.0 g, 100% purity, 10.1 mmol) was dissolved in a mixture of tetrahydrofuran (10 mL) and methanol (20 mL), then the solution was cooled to 0° C. and sodium tetrahydroborate (305 mg, 8.06 mmol) was added. The reaction was allowed to warm to room temperature and stirred for 2 hours, then quenched by the addition of water and extracted with ethyl acetate (40 mL) for 3 times. The organic layers were combined, washed with brine, dried over anhydrous Na₂SO₄₍ₛ₎ and concentrated under reduced pressure to give the title compound (1.9 g, 95% purity from ¹H NMR, 89% yield) as white solids. ¹H NMR (DMSO-d₆, 400 MHz) δ 7.87 (d, J=8.8 Hz, 2H), 7.60 (d, J=8.0 Hz, 2H), 5.40 (d, J=4.0 Hz, 1H), 4.85-4.80 (m, 1H), 3.19 (s, 3H), 1.34 (d, J=6.4 Hz, 3H).

Intermediate 18-3

1-(1-Bromoethyl)-4-(methylsulfonyl)benzene

To a solution of 1-(4-(methylsulfonyl)phenyl)ethanol 18-2 (1.6 g, 95% purity, 7.59 mmol) in tetrahydrofuran (20 mL) was added perbromomethane (4.5 g, 13.6 mmol) and triphenylphosphine (3.6 g, 13.7 mmol) at room temperature. After being stirred for 3 hours at 40° C. under nitrogen atmosphere, the mixture was concentrated under reduced pressure, then was purified by silica gel column chromatography (petroleum ether:ethyl acetate=1000:1 to 1:1) to give the title compound (1.8 g, 95% purity from ¹H NMR, 86% yield) as white solids. ¹H NMR (400 MHz, DMSO-d₆): δ 7.92 (d, J=8.4 Hz, 2H), 7.78 (d, J=8.0 Hz, 2H), 5.58 (q, J=6.8 Hz, 1H), 3.23 (s, 3H), 2.00 (d, J=6.8 Hz, 3H).

Intermediate 18-4

(3R,7S)-2-(3,4-Dichlorobenzoyl)-7-(hydroxymethyl)-3-methyl-9-(1-(4-(methylsulfonyl)phenyl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (600 mg, 95% purity, 0.880 mmol) and 1-(1-bromoethyl)-4-(methylsulfonyl)benzene 18-3 (425 mg, 95% purity, 1.53 mmol) in tetrahydrofuran (6 mL) at 0° C. was added benzyltriethylammonium chloride (208 mg, 0.913 mmol) and saturated sodium hydroxide aqueous solution (4 mL) dropwise. After being stirred at room temperature for 2 hours, water (30 mL) was added, extracted with ethyl acetate (60 mL). The combined organic layers were concentrated under reduced pressure, the resulting residue was purified by silica gel column chromatography (dichloromethane:methanol=100:1 to 16:1) to give the title compound (400 mg, 100% purity from LCMS, 77% yield) as white solids. LC-MS (ESI): R_{T}=1.49 min, mass calcd. for C₂₇H₂₈Cl₂N₄O₅S 590.1, m/z found 591.1 [M+H]⁺.

Intermediate 18-5

(3R,7S)-2-(3,4-Dichlorobenzoyl)-3-methyl-9-(1-(4-(methylsulfonyl)phenyl)ethyl)-10-oxo-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-7-(hydroxymethyl)-3-methyl-9-(1-(4-(methylsulfonyl)phenyl)ethyl)-1,2,3,4,8,9-hexahydropyrido[4′,3′:3,4]pyrazolo[1,5-a]pyrazin-10(7H)-one 18-4 (400 mg, 100% purity, 0.676 mmol) in acetonitrile (6.0 mL) was added saturated potassium dihydrogenphosphate aqueous solution (4.0 mL), sodium chlorite (153 mg, 1.35 mmol), 2,2,6,6-tetramethylpiperidinooxy (213 mg, 1.36 mmol) and sodium hypochlorite aqueous solution (1.5 mL, 1.39 mmol) at 0° C. After being stirred at 0° C. for 1 hour and stirred at room temperature for 2 hours, the mixture was diluted with sodium sulfite saturated solution (5 mL), acidified to pH=3 with 1 M hydrochloride aqueous solution and extracted with ethyl acetate (20 mL) for three times. The combined organic layers were washed with brine (15 mL), dried over Na₂SO₄₍ₛ₎, filtered and concentrated to give the desired product (370 mg, 100% purity from LCMS, 90% yield) as white solids. LC-MS (ESI): R_{T}=1.30 min, mass calcd. for C₂₇H₂₆Cl₂N₄O₆S 604.1, m/z found 605.1 [M+H]⁺.

Compound 18

(3R,7S)-2-(3,4-dichlorobenzoyl)-N,3-dimethyl-9-(1-(4-(methylsulfonyl)phenyl)ethyl)-10-oxo-1,2,3,4,7,8,9,10-octahydropyrido[4′,3′:3,4]pyrazolo[1,5-a]pyrazine-7-carboxamide′

A racemic mixture of (3R,7S)-2-(3,4-dichlorobenzoyl)-3-methyl-9-(1-(4-(methyl sulfonyl)phenyl)ethyl)-10-oxo-1,2,3,4,7,8,9,10-octahydropyrido[4′,3′:3,4]pyrazolo[1,5-a]pyrazine-7-carboxylic acid 18-5 (320 mg, 100% purity, 0.528 mol), methylamine hydrochloride (89 mg, 1.32 mmol), 1-(3-dimethylaminopropyl)-3-ethylcarbodiimide hydrochloride (203 mg, 1.06 mmol) and 1H-benzo[d][1,2,3]triazol-1-ol (143 mg, 1.06 mmol) in N,N-dimethylformamide (4 mL) was added trimethylamine (240 mg, 2.37 mmol) at 0° C. dropwise. After being stirred at room temperature under nitrogen atmosphere for 2 hours, the mixture was acidified to pH=3 with 1 M hydrochloride aqueous solution and extracted with ethyl acetate (30 mL) for three times. The combined organic layers were concentrated under reduced pressure to give a residue, which was purified by C18 column (acetonitrile:water=5% to 80%) to give the title compound (270 mg, 100% purity LCMS, 83% yield) as white solids. LC-MS (ESI): R_{T}=1.40 min, mass calcd. for C₂₈H₂₉Cl₂N₅O₅S 617.1, m/z found 618.1 [M+H]⁺.

Compounds 18A and 18B (Preparation Method A)

(3R,7S)-2-(3,4-Dichlorobenzoyl)-N,3-dimethyl-9-((R)-1-(4-(methylsulfonyl)phenyl)ethyl)-10-oxo-1,2,3,4,7,8,9,10-octahydropyrido[4′,3′:3,4]pyrazolo[1,5-a]pyrazine-7-carboxamide (18A), and (3R,7S)-2-(3,4-dichlorobenzoyl)-N,3-dimethyl-9-((S)-1-(4-(methylsulfonyl)phenyl)ethyl)-10-oxo-1,2,3,4,7,8,9,10-octahydropyrido[4′,3′:3,4]pyrazolo[1,5-a]pyrazine-7-carboxamide (18B)

The racemate of (3R,7S)-2-(3,4-dichlorobenzoyl)-N,3-dimethyl-9-(1-(4-(methylsulfonyl)phenyl)ethyl)-10-oxo-1,2,3,4,7,8,9,10-octahydropyrido[4′,3′:3,4]pyrazolo[1,5-a]pyrazine-7-carboxamide 1 (300 mg, 100% purity, 0.485 mmol) was separated by chiral Prep. HPLC (separation method: Column: Chiralpak IB, m 30*250 mm; Mobile Phase: Hexane:ethanol=30:70 at 60 mL/min; Col. Temp: 30° C.; Wavelength: 254 nm) to give the title products 18A (64.6 mg, 98.9% purity, 21% yield, 100% stereopure) and 18B (114.3 mg, 99.0% purity, 38% yield, 100% stereopure) as white solids.

18A

LC-MS (ESI): R_{T}=3.424 min, mass calcd. for C₂₈H₂₉Cl₂N₅O₅S 617.3, m/z found 618.0 [M+H]⁺. Chiral analysis (Column: Chiralpak IB, 5 μm 4.6*250 mm; Mobile Phase: Hexane:ethanol=30:70 at 1 mL/min; Temp: 30° C.; Wavelength: 254 nm; R_{T}=5.628 min). ¹H NMR (400 MHz, CDCl₃) δ 7.91 (d, J=8.8 Hz, 2H), 7.57-7.47 (m, 4H), 7.27-7.25 (m, 1H), 6.12-5.31 (m, 3H), 4.99-4.24 (m, 3H), 4.02-3.83 (m, 2H), 3.16-2.98 (m, 4H), 2.77-2.72 (m, 1H), 2.69 (d, J=5.2 Hz, 3H), 1.64 (d, J=7.2 Hz, 3H), 1.30 (d, J=6.8 Hz, 3H).

18B

LC-MS (ESI): R_{T}=3.199 min, mass calcd. for C₂₈H₂₉Cl₂N₅O₅S 617.3, m/z found 618.0 [M+H]⁺. Chiral analysis (Column: Chiralpak IB, 5 μm 4.6*250 mm; Mobile Phase: Hexane:ethanol=30:70 at 1 mL/min; Temp: 30° C.; Wavelength: 254 nm; R_{T}=8.082 min). ¹H NMR (400 MHz, CDCl₃) δ 7.93 (d, J=8.4 Hz, 2H), 7.62-7.50 (m, 4H), 7.28-7.23 (m, 1H), 6.26-5.29 (m, 3H), 4.85-4.26 (m, 3H), 4.12 (d, J=13.2 Hz, 1H), 3.43 (dd, J=13.2 and 4.8 Hz, 1H), 3.16-2.95 (m, 4H), 2.80 (d, J=4.4 Hz, 3H), 2.71 (d, J=16.0 Hz, 1H), 1.64 (d, J=6.8 Hz, 3H), 1.30 (d, J=6.8 Hz, 3H).

Compound 18B (Preparation Method B)

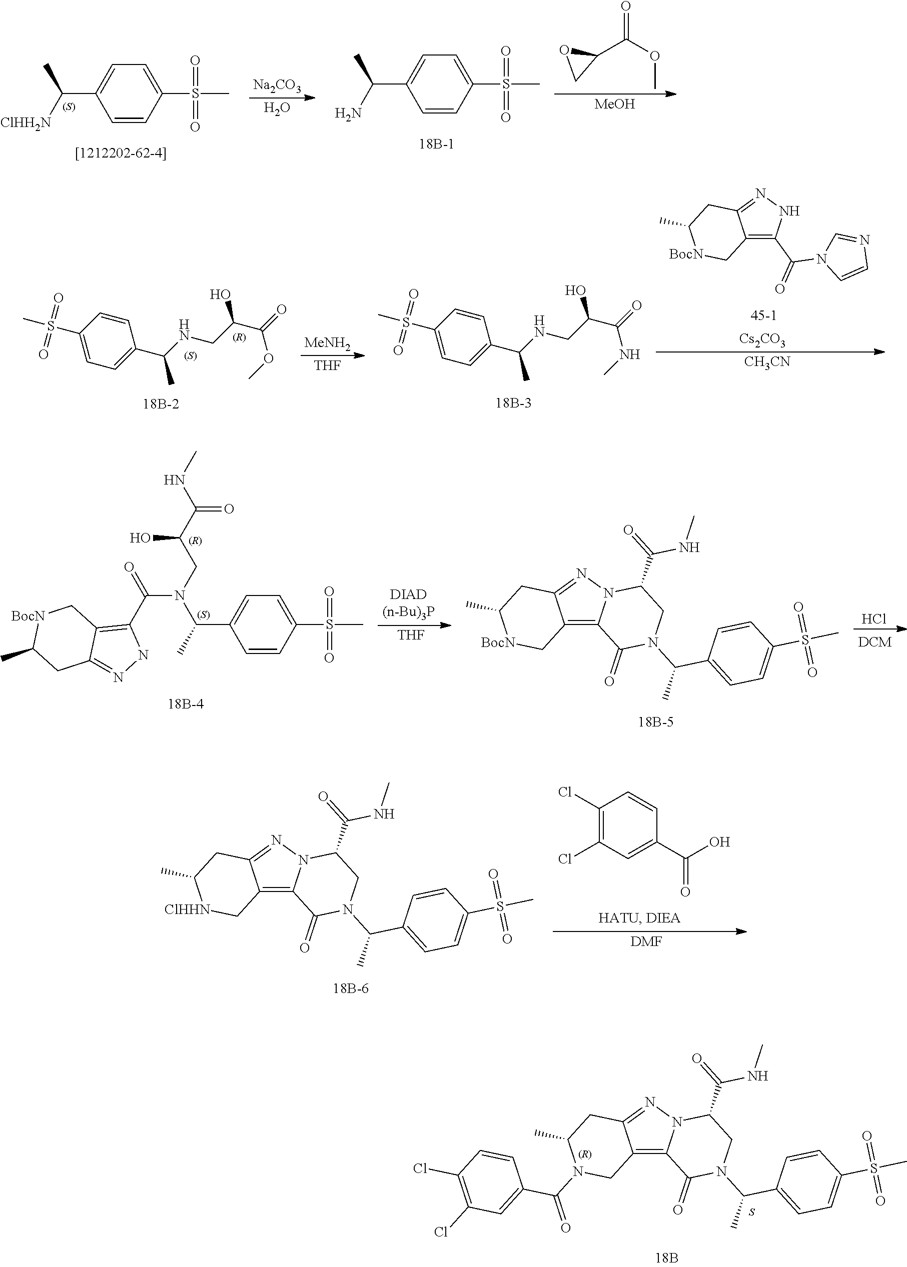

Intermediate 18B-1

(S)-1-(4-(Methylsulfonyl)phenyl)ethan-1-amine

(S)-1-(4-(methylsulfonyl)phenyl)ethan-1-amine hydrochloride (4.0 g, 17.0 mmol) was dissolved with saturated sodium carbonate aqueous solution (20 mL), then extracted with the solution of dichloromethane and methanol (10:1, 20 mL) for four times. The combined organic layers were dried over Na₂SO₄₍ₛ₎, filtered. The filtrate was concentrated to give the title compound (3.5 g, 95% yield from ¹H NMR, 98% yield) as yellow oil. ¹H NMR (400 MHz, CDCl₃) δ 7.90 (d, J=8.0 Hz, 2H), 7.57 (d, J=8.4 Hz, 2H), 4.24 (q, J=6.4 Hz, 1H), 3.05 (s, 3H), 1.40 (d, J=6.8 Hz, 3H).

Intermediate 18B-2

Methyl (R)-2-hydroxy-3-(((S)-1-(4-(methylsulfonyl)phenyl)ethyl)amino)propanoate

To the solution of (S)-1-(4-(methylsulfonyl)phenyl)ethan-1-amine 18B-1 (10.0 g, 42.4 mmol) in methanol (100 mL) was added methyl (R)-oxirane-2-carboxylate (4.7 g, 46.0 mmol) at room temperature. After stirred at 60° C. for 15 hours, the solution was concentrated to give the title compound (13.5 g, 84% purity from LCMS, 93% yield) as yellow oil. LC-MS (ESI): R_{T}=1.13 min, mass calcd. for C₁₃H₁₉NO₅S 301.1, m/z found 302.0 [M+H]⁺.

Intermediate 18B-3

(R)-2-Hydroxy-N-methyl-3-(((S)-1-(4-(methylsulfonyl)phenyl)ethyl)amino) propenamide

To a sealed tube loaded methyl (R)-2-hydroxy-3-(((S)-1-(4-(methylsulfonyl)phenyl) ethyl)amino)propanoate 18B-2 (13.5 g, 84% purity, 37.6 mmol) was added 2 M methylamine in tetrahydrofuran (60 mL, 120 mmol). After stirred at 70° C. for 24 hours, the mixture solution was concentrated to give the title compound (11.0 g, 90% purity from ¹H NMR, 84% yield) as colorless oil. ¹H NMR (400 MHz, CDCl₃) δ 7.89 (d, J=8.4 Hz, 2H), 7.48 (d, J=8.4 Hz, 2H), 6.97 (br s, 1H), 3.93 (t, J=5.2 Hz, 1H), 3.85 (q, J=6.4 Hz, 1H), 3.05 (s, 3H), 2.94-2.89 (m, 1H), 2.84-2.78 (m, 4H), 2.73-2.69 (m, 1H), 1.38 (d, J=6.4 Hz, 3H).

Intermediate 18B-4

tert-Butyl (R)-3-(((R)-2-hydroxy-3-(methylamino)-3-oxopropyl)((S)-1-(4-(methyl sulfonyl)phenyl)ethyl)carbamoyl)-6-methyl-2,4,6,7-tetrahydro-5H-pyrazolo[4,3-c]pyridine-5-carboxylate

To a solution of (R)-2-hydroxy-N-methyl-3-(((S)-1-(4-(methylsulfonyl)phenyl)ethyl) amino)propanamide 18B-3 (11.0 g, 90% purity, 33.0 mmol) and tert-butyl (R)-3-(1H-imidazole-1-carbonyl)-6-methyl-2,4,6,7-tetrahydro-5H-pyrazolo[4,3-c]pyridine-5-carboxylate 45-1 (12.5 g, 98% purity, 37.0 mmol) in acetonitrile (150 mL) was added cesium carbonate (16.5 g, 50.6 mmol). After stirred at 80° C. for 3 hours, the reaction mixture was filtered, concentrated. The filtrate was purified by silica gel column chromatography (dichloromethane:acetone=3:1) to give the title compound (10.8 g, 97% purity from LCMS, 56% yield) as white solids. LC-MS (ESI): R_{T}=1.33 min, mass calcd. for C₂₆H₃₇N₅O₇S 563.2, m/z found 564.1 [M+H]⁺.

Intermediate 18B-5

tert-Butyl (3R,7S)-3-methyl-7-(methylcarbamoyl)-9-((S)-1-(4-(methylsulfonyl)phenyl)ethyl)-10-oxo-3,4,7,8,9,10-hexahydropyrido[4′,3′:3,4]pyrazolo[1,5-a]pyrazine-2(1H)-carboxylate

To a solution of tert-butyl (R)-3-(((R)-2-hydroxy-3-(methylamino)-3-oxopropyl)((S)-1-(4-(methylsulfonyl)phenyl)ethyl)carbamoyl)-6-methyl-2,4,6,7-tetrahydro-5H-pyrazolo[4,3-c]pyridine-5-carboxylate 18B-4 (10.8 g, 97% purity, 18.6 mmol) in tetrahydrofuran (300 mL) was added tributylphosphine (7 mL, 27.5 mmol) and diisopropyl azodicarboxylate (5.5 mL, 28.0 mmol) at 0° C. under nitrogen atmosphere. After stirred at room temperature for 1 hour, the mixture was concentrated, purified by silica gel column chromatography (dichloromethane:acetonitrile=3:1) to give the title compound (9.5 g, 100% purity from LCMS, 94% yield) as white solids. LC-MS (ESI): R_{T}=1.39 min, mass calcd. for C₂₆H₃₅N₅O₆S 545.2, m/z found 544.0 [M−H]⁻.

Intermediate 18B-6

(3R,7S)—N,3-Dimethyl-9-((S)-1-(4-(methylsulfonyl)phenyl)ethyl)-10-oxo-1,2,3,4,7,8,9,10-octahydropyrido[4′,3′:3,4]pyrazolo[1,5-a]pyrazine-7-carboxamide hydrochloride

To a solution of tert-butyl (3R,7S)-3-methyl-7-(methylcarbamoyl)-9-((S)-1-(4-(methyl sulfonyl)phenyl)ethyl)-10-oxo-3,4,7,8,9,10-hexahydropyrido[4′,3′:3,4]pyrazolo[1,5-a]pyrazine-2(1H)-carboxylate 18B-5 (9.5 g, 100% purity, 17.4 mmol) in dichloromethane (30 mL) was added 4 M hydrochloride in 1,4-dioxane (70 mL, 280 mmol) at 0° C. After stirred for 1 hour, the mixture was concentrated to give the colorless oil. The residues were washed with ethyl acetate (50 mL), dissolved in water (50 mL), then extracted with ethyl acetate (50 mL) twice. The aqueous layers were concentrated to give the title compound (8.0 g, 81% purity from LCMS, 77% yield) as off-white solids. LC-MS (ESI): R_{T}=1.01 min, mass calcd. for C₂₁H₂₇N₅O₄S 445.2, m/z found 446.0 [M+H]⁺.

Compound 18B

(3R,7S)-2-(3,4-Dichlorobenzoyl)-N,3-dimethyl-9-((S)-1-(4-(methylsulfonyl)phenyl) ethyl)-10-oxo-1,2,3,4,7,8,9,10-octahydropyrido[4′,3′:3,4]pyrazolo[1,5-a]pyrazine-7-carboxamide

To the solution of (3R,7S)—N,3-dimethyl-9-((S)-1-(4-(methylsulfonyl)phenyl) ethyl)-10-oxo-1,2,3,4,7,8,9,10-octahydropyrido[4′,3′:3,4]pyrazolo[1,5-a]pyrazine-7-carboxamide hydrochloride 18B-6 (8.0 g, 81% purity, 13.4 mmol) and 3,4-dichlorobenzoic acid (3.8 g, 20.3 mmol) in N,N-dimethylformamide (100 mL) were added N,N-diisopropylethylamine (7 mL, 40.2 mmol) and O-(7-azabenzotriazol-1-yl)-N,N,N′,N′-tetramethyluronium hexafluorophosphate (7.7 g, 20.3 mmol). After stirred at room temperature for 1 hour, the mixture was diluted with water (200 mL), extracted with ethyl acetate (150 mL) twice. The combined organic layers were washed with water (200 mL) and brine (100 mL), dried over Na₂SO₄₍ₛ₎, filtered. The filtrate was concentrated, purified by C18 column (acetonitrile:water=50% to 70%) to give the title compound (7.5 g, 65% purity from SFC, 59% yield) as white solids. LC-MS (ESI): R_{T}=1.42 min, mass calcd. for C₂₈H₂₉Cl₂N₅O₅S 617.1, m/z found 618.0 [M+H]⁺. Chiral analysis (Column: Chiralpak IH 5 μm 4.6*250 mm; Mobile Phase: CO₂:ACN=60:40, at 4 mL/min; Col. Temp: 40° C.; Wavelength: 230 nm; Rt=11.38 min). The crude 18B (7.5 g, 65% purity, 7.88 mmol) was purified by chiral Prep. HPLC (separation condition: Column: Chiralpak IH 5 μm 30*250 mm; Mobile Phase: CO₂:ACN=60:40 at 25 mL/min; Col. Temp: 40° C.; Wavelength: 230 nm) to give the title compound (3.8 g, 99.6% purity from LCMS, 78% yield, 99.6% stereopure) as white solids. LC-MS (ESI): R_{T}=9.292 min, mass calcd. for C₂₈H₂₉Cl₂N₅O₅S 617.3, m/z found 618.0 [M+H]⁺. Chiral analysis (Column: Chiralpak IH 5 μm 4.6*250 mm; Mobile Phase: CO₂:ACN=60:40 at 4 mL/min; Col. Temp: 40° C.; Wavelength: 230 nm; Rt=11.49 min). H NMR (400 MHz, CDCl₃) δ 7.92 (d, J=8.0 Hz, 2H), 7.57-7.51 (m, 4H), 7.27-7.25 (m, 1H), 6.02-5.33 (m, 3H), 4.85-4.38 (m, 3H), 4.11 (d, J=12.8 Hz, 1H), 3.43 (dd, J=13.2 and 4.8 Hz, 1H), 3.12-2.94 (m, 4H), 2.80 (d, J=4.8 Hz, 3H), 2.71 (d, J=16.4 Hz, 1H), 1.63 (d, J=7.2 Hz, 3H), 1.29 (d, J=6.8 Hz, 3H).

Compounds 19A and 19B

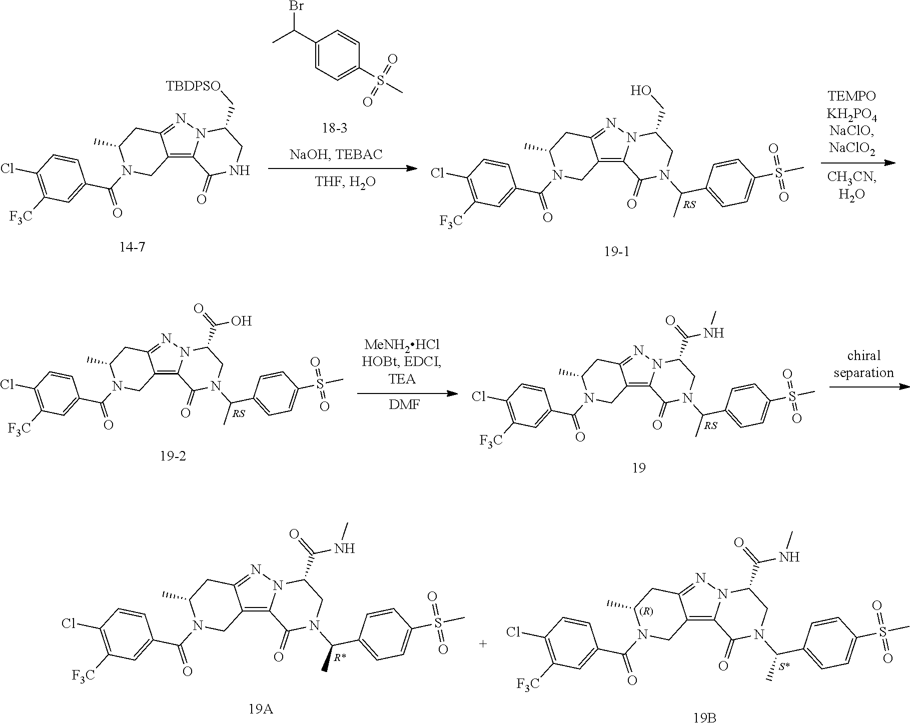

Intermediate 19-1

(3R,7S)-2-(4-Chloro-3-(trifluoromethyl)benzoyl)-7-(hydroxymethyl)-3-methyl-9-(1-(4-(methylsulfonyl)phenyl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(4-chloro-3-(trifluoromethyl)benzoyl)-3-methyl-1,2,3,4,8,9-hexahydropyrido[4′,3′:3,4]pyrazolo[1,5-a]pyrazin-10(7H)-one 14-7 (3.2 g, 100% purity, 4.70 mmol) and 1-(1-bromoethyl)-4-(methylsulfonyl)benzene 18-3 (2.28 g, 95% purity, 8.23 mmol) in tetrahydrofuran (30 mL) at 0° C. was added benzyltriethylammonium chloride (1.1 g, 4.83 mmol) and dropwise saturated sodium hydroxide aqueous solution (25 mL). After being stirred at room temperature for 2 hours, the mixture was added water (80 mL) and extracted with ethyl acetate (20 mL) for three times. The combined organic layers were concentrated under reduced pressure. The resulting residue was purified by C18 column (acetonitrile:water=5% to 65%) to give the title compound (1.65 g, 96% purity from LCMS, 54% yield) as white solids. LC-MS (ESI): R_{T}=1.44 min and 1.46 min, mass calcd. for C₂₈H₂₈ClF₃N₄O₅S 624.1, m/z found 625.0 [M+H]⁺.

Intermediate 19-2

(3R,7S)-2-(4-Chloro-3-(trifluoromethyl)benzoyl)-3-methyl-9-(1-(4-(methylsulfonyl)phenyl)ethyl)-10-oxo-1,2,3,4,7,8,9,10-octahydropyrido[4′,3′:3,4]pyrazolo[1,5-a]pyrazine-7-carboxylic acid

To a solution of (3R,7S)-2-(4-chloro-3-(trifluoromethyl)benzoyl)-7-(hydroxymethyl)-3-methyl-9-(1-(4-(methylsulfonyl)phenyl)ethyl)-1,2,3,4,8,9-hexahydropyrido[4′,3′:3,4]pyrazolo[1,5-a]pyrazin-10(7H)-one 19-1 (200 mg, 97% purity, 0.307 mmol) in acetonitrile (2.0 mL) was added saturated potassium dihydrogenphosphate aqueous solution (2.0 mL), sodium chlorite (69 mg, 0.610 mmol), 2,2,6,6-tetramethylpiperidinooxy (97 mg, 0.617 mmol) and sodium hypochlorite aqueous solution (0.7 mL, 0.646 mmol) at 0° C. After being stirred at 0° C. for 1 hour and stirred at room temperature for 2 hours, the mixture was diluted with sodium sulfite saturated solution (20 mL). The mixture was acidified to pH=3 with 1 M hydrochloride aqueous solution and extracted with ethyl acetate (20 mL) for three times. The combined organic layers were washed with brine (15 mL), dried over Na₂SO₄₍ₛ₎ and filtered. The filtrate was concentrated to give the desired product (200 mg, 92% purity from LCMS, 94% yield) as white solids. LC-MS (ESI): R_{T}=2.08 min and 2.11 min, mass calcd. for C₂₈H₂₆ClF₃N₄O₆S 638.1, m/z found 639.0 [M+H]⁺.

Compound 19

(3R,7S)-2-(4-Chloro-3-(trifluoromethyl)benzoyl)-N,3-dimethyl-9-(1-(4-(methylsulfonyl)phenyl)ethyl)-10-oxo-1,2,3,4,7,8,9,10-octahydropyrido[4′,3′:3,4]pyrazolo[1,5-a]pyrazine-7-carboxamide

To the mixture of (3R,7S)-2-(4-chloro-3-(trifluoromethyl)benzoyl)-3-methyl-9-(1-(4-(methylsulfonyl)phenyl)ethyl)-10-oxo-1,2,3,4,7,8,9,10-octahydropyrido[4′,3′:3,4]pyrazolo[1,5-a]pyrazine-7-carboxylic acid 19-2 (200 mg, 92% purity, 0.288 mmol), methanamine hydrochloride (47 mg, 0.696 mmol), 1-(3-dimethylaminopropyl)-3-ethylcarbodiimide hydrochloride (110 mg, 0.574 mmol) and 1H-benzo[d][1,2,3]triazol-1-ol (78 mg, 0.577 mmol) in N,N-dimethylformamide (4 mL) at 0° C. was added trimethylamine (131 mg, 1.30 mmol) dropwise. After being stirred at 0° C. under nitrogen atmosphere for 1 hour, the mixture was acidified to pH=5 with 0.5 M hydrochloride aqueous solution and extracted with ethyl acetate (10 mL) twice. The combined organic layers were concentrated under reduced pressure to give a residue, which was purified by C18 column (acetonitrile:water=05% to 65%) to give the title compound (135 mg, 100% purity LCMS, 72% yield) as white solids. LC-MS (ESI): R_{T}=1.70 min, mass calcd. for C₂₉H₂₉ClF₃N₅O₅S 651.2, m/z found 652.0 [M+H]⁺.

Compounds 19A and 19B

(3R,7S)-2-(4-Chloro-3-(trifluoromethyl)benzoyl)-N,3-dimethyl-9-((R*)-1-(4-(methylsulfonyl)phenyl)ethyl)-10-oxo-1,2,3,4,7,8,9,10-octahydropyrido[4′,3′:3,4]pyrazolo[1,5-a]pyrazine-7-carboxamide (19A), and (3R,7S)-2-(4-chloro-3-(trifluoromethyl)benzoyl)-N,3-dimethyl-9-((S*)-1-(4-(methylsulfonyl)phenyl)ethyl)-10-oxo-1,2,3,4,7,8,9,10-octahydropyrido[4′,3′:3,4]pyrazolo[1,5-a]pyrazine-7-carboxamide (19B)

(3R,7S)-2-(4-chloro-3-(trifluoromethyl)benzoyl)-N,3-dimethyl-9-(1-(4-(methylsulfonyl)phenyl)ethyl)-10-oxo-1,2,3,4,7,8,9,10-octahydropyrido[4′,3′:3,4]pyrazolo[1,5-a]pyrazine-7-carboxamide 19 (170 mg, 100% purity, 0.261 mmol) was separated by chiral Prep. HPLC (separation method: Column: Chiralpak IB N-5, 5 m 30*250 mm; Mobile Phase: acetonitrile: isopropyl alcohol: =90:10 at 60 mL/min; Col. Temp: 30° C.; Wavelength: 254 nm) to give the title compound 19A (32.4 mg, 96.6% purity, 18% yield, 99.9% stereopure) as white solids and 19B (82.9 mg, 99.8% purity, 49% yield, 99.9% stereopure) as white solids.

19A

LC-MS (ESI): R_{T}=3.424 min, mass calcd. for C₂₉H₂₉ClF₃N₅O₅S 651.2, m/z found 652.2 [M+H]⁺. Chiral analysis (Column: Chiralpak IB N-5, 5 m 4.6*250 mm; Mobile Phase: acetonitrile: isopropyl alcohol=90:10 at 1 mL/min; Temp: 30° C.; Wavelength: 254 nm; R_{T}=3.730 min). ¹H NMR (400 MHz, CDCl₃) δ 7.91 (d, J=8.4 Hz, 2H), 7.77 (d, J=1.6 Hz, 1H), 7.60-7.50 (m, 4H), 6.17-5.35 (m, 3H), 4.93-4.34 (m, 3H), 3.99-3.86 (m, 2H), 3.18-2.98 (m, 4H), 2.81-2.64 (m, 4H), 1.64 (d, J=7.2 Hz, 3H), 1.32 (d, J=6.8 Hz, 3H). ¹⁹F NMR (376 MHz, CDCl₃) δ −62.84.

19B

LC-MS (ESI): R_{T}=3.424 min, mass calcd. for C₂₉H₂₉ClF₃N₅O₅S 651.2, m/z found 652.2 [M+H]⁺. Chiral analysis (Column: Chiralpak TB N-5, 5 m 4.6*250 mm; Mobile Phase: acetonitrile: isopropyl alcohol=90:10 at 1 mL/min; Temp: 30° C.; Wavelength: 254 nm; R_{T}=4.882 min). ¹H NMR (400 MHz, CDCl₃) δ 7.93 (d, J=8.0 Hz, 2H), 7.77 (d, J=1.6 Hz, 1H), 7.61-7.52 (m, 4H), 6.28-5.33 (m, 3H), 4.98-4.28 (m, 3H), 4.13 (d, J=10.8 Hz, 1H), 3.44 (dd, J=13.2 and 4.8 Hz, 1H), 3.15-2.99 (m, 4H), 2.81 (d, J=4.8 Hz, 3H), 2.73 (d, J=15.6 Hz, 1H), 1.64 (d, J=7.2 Hz, 3H), 1.31 (d, J=6.4 Hz, 3H). ¹⁹F NMR (376 MHz, CDCl₃) δ −62.83.

Compounds 20A and 20B and 21

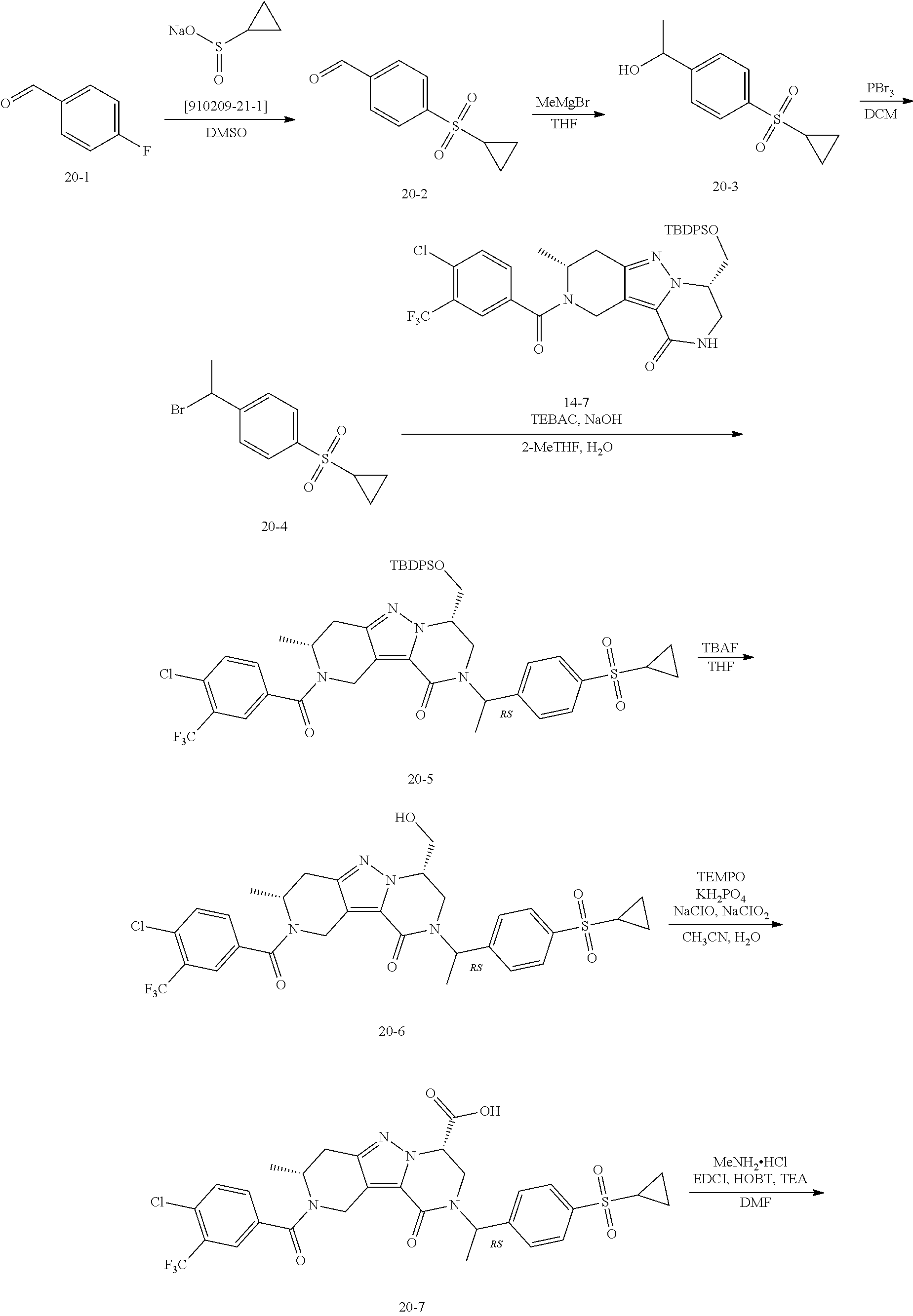

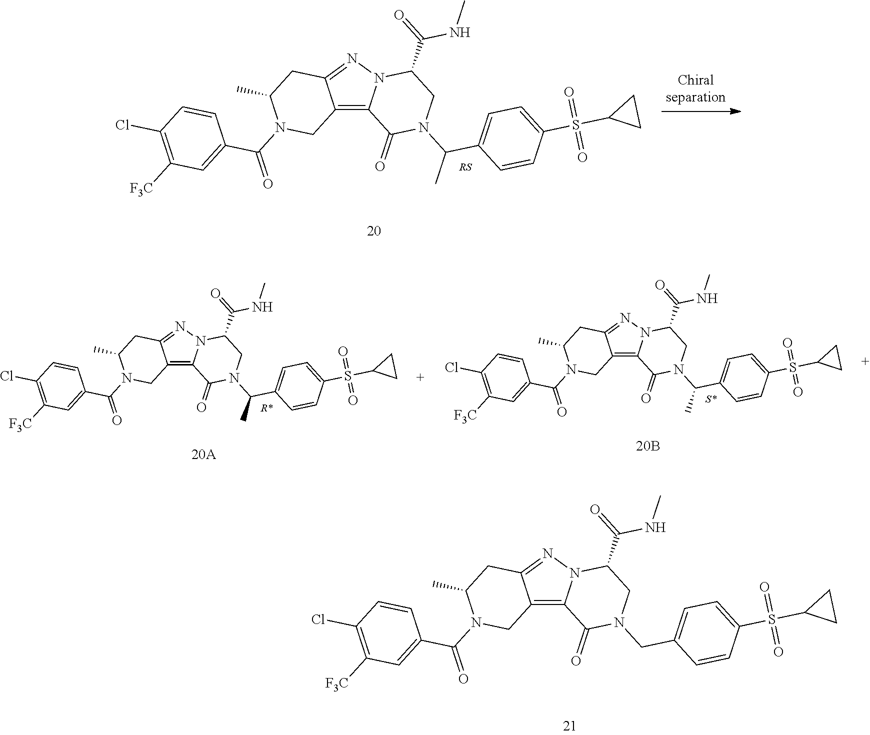 [SMILES: CNC(=O)[C@@H]1CN(C(C)c2ccc(S(=O)(=O)C3CC3)cc2)C(=O)c2c3c(nn21)C[C@@H](C)N(C(=O)c1ccc(Cl)c(C(F)(F)F)c1)C3 ; CNC(=O)[C@@H]1CN([C@H](C)c2ccc(S(=O)(=O)C3CC3)cc2)C(=O)c2c3c(nn21)C[C@@H](C)N(C(=O)c1ccc(Cl)c(C(F)(F)F)c1)C3 ; CNC(=O)[C@@H]1CN([C@@H](C)c2ccc(S(=O)(=O)C3CC3)cc2)C(=O)c2c3c(nn21)C[C@@H](C)N(C(=O)c1ccc(Cl)c(C(F)(F)F)c1)C3 ; CNC(=O)[C@@H]1CN(Cc2ccc(S(=O)(=O)C3CC3)cc2)C(=O)c2c3c(nn21)C[C@@H](C)N(C(=O)c1ccc(Cl)c(C(F)(F)F)c1)C3]

Intermediate 20-2

4-(Cyclopropylsulfonyl)benzaldehyde

To a solution of 4-fluorobenzaldehyde 20-1 (300 mg, 2.42 mmol) in dimethyl sulfoxide (12 ml) was added cyclopropanesulfinic acid sodium salt (370 mg, 2.89 mmol). The reaction mixture was put on microwave irradiation oven and heated at 140° C. for 1 hour. The reaction mixture was quenched with water (10 mL), extracted with ethyl acetate (10 mL) for three times, dried over Na₂SO₄₍ₛ₎, filtered. The filtrate was concentrated under reduced pressure to give the title product (500 mg, 90% purity from ¹H NMR, 88.5% yield) as yellow oil. ¹H NMR (300 MHz, CDCl₃) δ 10.17 (s, 1H), 8.18-8.06 (m, 4H), 2.60-2.47 (m, 1H), 1.50-1.39 (m, 2H), 1.19-1.06 (m, 2H).

Intermediate 20-3

1-(4-(Cyclopropylsulfonyl)phenyl)ethan-1-ol

To a solution of 4-(cyclopropylsulfonyl)benzaldehyde 20-2 (400 mg, 90% purity, 1.71 mmol) in dry tetrahydrofuran (4 mL) was added dropwise 1 M methylmagnesium bromide in tetrahydrofuran (4 mL, 4 mmol) at 0° C. under a nitrogen atmosphere. After being stirred at 0° C. for 2 hours, the reaction mixture was quenched with saturated ammonium chloride aqueous solution (20 mL), extracted with ethyl acetate (20 mL) for three times, dried over Na₂SO₄₍ₛ₎ and filtered. The filtrate was concentrated to give the title compound (400 mg, 90% purity from ¹H NMR, 92.9% yield) as colorless oil. ¹H NMR (300 MHz, CDCl₃) δ 7.92 (d, J=8.4 Hz, 2H), 7.62 (d, J=8.1 Hz, 2H), 5.09-5.03 (m, 1H), 2.60-2.44 (m, 1H), 1.84-1.67 (m, 1H), 1.58 (d, J=6.6 Hz, 3H), 1.47-1.36 (m, 2H), 1.10-1.08 (m, 2H).

Intermediate 20-4

1-(1-Bromoethyl)-4-(cyclopropylsulfonyl)benzene

To a solution of 1-(4-(cyclopropylsulfonyl)phenyl)ethan-1-ol 20-3 (450 mg, 90% purity, 1.79 mmol) in dichloromethane (5 mL) was added dropwise tribromophosphine (450 mg, 1.66 mmol) in dichloromethane (5 mL) at 0° C. The resulting mixture was stirred at 0° C. for 2 hours, then was poured into water (20 mL), and extracted with dichloromethane (20 mL) for three times. The combined organic layers were washed with brine (10 mL), dried over Na₂SO₄₍ₛ₎ and filtered. The filtrate was concentrated in vacuum to give the title compound (500 mg, 80% purity from ¹H NMR, 77.3% yield) as yellow oil. ¹H NMR (300 MHz, CDCl₃) δ 8.01-7.91 (m, 2H), 7.71-7.55 (m, 2H), 5.83-5.53 (m, 1H), 2.60-2.43 (m, 1H), 2.43-2.29 (m, 3H), 1.48-1.36 (m, 2H), 1.11-1.09 (m, 2H).

Intermediate 20-5

(3R,7S)-7-(((tert-Butyldiphenylsilyl)oxy)methyl)-2-(4-chloro-3-(trifluoromethyl) benzoyl)-9-(1-(4-(cyclopropylsulfonyl)phenyl)ethyl)-3-methyl-1,2,3,4,8,9-hexa hydropyrido[4′,3′:3,4]pyrazolo[1,5-a]pyrazin-10(7H)-one

To a solution of (3R,7S)-7-(((tert-butyldiphenylsilyl)oxy)methyl)-2-(4-chloro-3-(trifluoromethyl)benzoyl)-3-methyl-1,2,3,4,8,9-hexahydropyrido[4′,3′:3,4]pyrazolo[1,5-a]pyrazin-10(7H)-one 14-7 (420 mg, 100% yield, 0.62 mmol) and 1-(1-bromoethyl)-4-(cyclopropylsulfonyl)benzene 20-4 (320 mg, 80% purity, 0.89 mmol) in 2-methyltetrahydrofuran (4 mL) were added sodium hydroxide in water (2 mL, 50% wt) and benzyltriethylammonium chloride (20 mg, 0.09 mmol). After being stirred at 50° C. for 2 hours, the reaction was poured into water (10 mL), extracted with ethyl acetate (10 mL) for three times. The combined organic layers were washed with brine (10 mL), dried over Na₂SO₄₍ₛ₎ and filtered. The filtrate was concentrated in vacuum to give the title compound (750 mg, 45% purity from LCMS, 61.5% yield) as yellow solids. LC-MS (ESI): R_{T}=1.58 min, 2.08 min and 2.16 min, mass calcd. for C₄₆H₄₈ClF₃N₄O₅SSi 888.3, m/z found 668.0 [M−TBDPS+H₃O]⁺, 889.1 [M+H]⁺.

Intermediate 20-6

(3R,7S)-2-(4-Chloro-3-(trifluoromethyl)benzoyl)-9-(1-(4-(cyclopropylsulfonyl) phenyl)ethyl)-7-(hydroxymethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(4-chloro-3-(trifluoromethyl)benzoyl)-9-(1-(4-(cyclopropylsulfonyl)phenyl)ethyl)-3-methyl-1,2,3,4,8,9-hexahydropyrido[4′,3′:3,4]pyrazolo[1,5-a]pyrazin-10(7H)-one 20-5 (750 mg, 45% purity, 0.38 mmol) in tetrahydrofuran (10 mL) was added 1 M tetrabutylammonium fluoride solution in tetrahydrofuran (0.4 mL, 0.4 mmol). After being stirred at 20° C. for 2 hours, the reaction was poured into saturated ammonium chloride aqueous solution (10 mL), extracted with ethyl acetate (20 mL) for three times. The combined organic layers were washed with brine (20 mL), dried over Na₂SO₄₍ₛ₎, and filtered. The filtrate was concentrated in vacuum, which was purified by C18 chromatography (acetonitrile:water (+0.02% ammonium bicarbonate)=40-60%) to give the title compound (220 mg, 100% purity from LCMS, 89.1% yield) as white solids. LC-MS (ESI): R_{T}=1.57 min, mass calcd. for C₃₀H₃₀ClF₃N₄O₅S 650.2, m/z found 668.0 [M+H₃O]⁺.

Intermediate 20-7

(3R,7S)-2-(4-Chloro-3-(trifluoromethyl)benzoyl)-9-(1-(4-(cyclopropylsulfonyl) phenyl)ethyl)-3-methyl-10-oxo-1,2,3,4,7,8,9,10-octahydropyrido[4′,3′:3,4] pyrazolo[1,5-a]pyrazine-7-carboxylic acid

To a solution of (3R,7S)-2-(4-chloro-3-(trifluoromethyl)benzoyl)-9-(1-(4-(cyclopropylsulfonyl)phenyl)ethyl)-7-(hydroxymethyl)-3-methyl-1,2,3,4,8,9-hexahydropyrido[4′,3′:3,4]pyrazolo[1,5-a]pyrazin-10(7H)-one 20-6 (220 mg, 100% purity, 0.34 mmol) in acetonitrile (2 mL) were added saturated potassium dihydrogen phosphate aqueous solution (2 mL), 2,2,6,6-tetramethylpiperidinooxy (110 mg, 0.70 mmol), sodium chlorite (80 mg, 0.71 mmol) and dropwise 10% sodium hypochlorite aqueous solution (500 mg, 0.67 mmol) at 0° C. The reaction was allowed to slowly return to room temperature. After being stirred at room temperature for 3 hours, the reaction mixture was quenched with saturated sodium thiosulfate (2 mL), extracted with ethyl acetate (10 mL) for three times. The combined organic layers were washed with brine (10 mL), dried over Na₂SO₄₍ₛ₎ and filtered. The filtrate was concentrated to get a residue, which was purified by C18 chromatography (acetonitrile:water (+0.02% ammonium bicarbonate)=35-55%) to give the title compound (190 mg, 100% purity from LCMS, 84.5% yield) as white solids. LC-MS (ESI): R_{T}=1.31 min, mass calcd. for C₃₀H₂₈ClF₃N₄O₆S 664.1, m/z found 665.6 [M+H]⁺.

Compound 20

(3R,7S)-2-(4-Chloro-3-(trifluoromethyl)benzoyl)-9-(1-(4-(cyclopropylsulfonyl) phenyl)ethyl)-N,3-dimethyl-10-oxo-1,2,3,4,7,8,9,10-octahydropyrido[4′,3′:3,4]pyrazolo[1,5-a]pyrazine-7-carboxamide

To a solution of (3R,7S)-2-(4-chloro-3-(trifluoromethyl)benzoyl)-9-(1-(4-(cyclopropylsulfonyl)phenyl)ethyl)-3-methyl-10-oxo-1,2,3,4,7,8,9,10-octahydropyrido[4′,3′:3,4]pyrazolo[1,5-a]pyrazine-7-carboxylic acid 20-7 (190 mg, 100% purity, 0.29 mmol), 1-(3-dimethylaminopropyl)-3-ethylcarbodiimide hydrochloride (110 mg, 0.57 mmol), 1H-benzo[d][1,2,3]triazol-1-ol (80 mg, 0.59 mmol) and methylamine hydrochloride (60 mg, 0.89 mmol) in N,N-dimethylformamide (2 mL) was added dropwise trimethylamine (190 mg, 1.88 mmol) in N,N-dimethylformamide (2 mL) at 0° C. After being stirred at 0° C. for 2 hours, the mixture was poured into water (10 mL), extracted with ethyl acetate (10 mL) for three times. The combined organic layers were washed with brine (10 mL), dried over Na₂SO₄₍ₛ₎ and filtered. The filtrate was concentrated in vacuum to give a residue, which was purified by C18 chromatography (acetonitrile:water (+0.02% ammonium bicarbonate)=40-60%) to give the title compound (170 mg, 94% purity from LCMS, 82.5% yield) as white solids. Comments: Chiral showed 88.3% 20 and 11.7% 21. LC-MS (ESI): R_{T}=1.56 min, mass calcd. for C₃₁H₃₁ClF₃N₅O₅S 677.2, m/z found 676.6 [M−H]⁻.

Compounds 20A and 20B and 21

(3R,7S)-2-(4-Chloro-3-(trifluoromethyl)benzoyl)-9-((R*)-1-(4-(cyclopropylsulfonyl)phenyl)ethyl)-N,3-dimethyl-10-oxo-1,2,3,4,7,8,9,10-octahydropyrido[4′,3′:3,4]pyrazolo[1,5-a]pyrazine-7-carboxamide (20A), and (3R,7S)-2-(4-Chloro-3-(trifluoromethyl)benzoyl)-9-((S*)-1-(4-(cyclopropylsulfonyl)phenyl)ethyl)-N,3-dimethyl-10-oxo-1,2,3,4,7,8,9,10-octahydropyrido[4′,3′:3,4]pyrazolo[1,5-a]pyrazine-7-carboxamide (20B), and (3R,7S)-2-(4-Chloro-3-(trifluoromethyl)benzoyl)-9-(4-(cyclopropylsulfonyl) benzyl)-N,3-dimethyl-10-oxo-1,2,3,4,7,8,9,10-octahydropyrido[4′,3′:3,4]pyrazolo[1,5-a]pyrazine-7-carboxamide (21)

The racemate (3R,7S)-2-(4-chloro-3-(trifluoromethyl)benzoyl)-9-(1-(4-(cyclopropylsulfonyl)phenyl)ethyl)-N,3-dimethyl-10-oxo-1,2,3,4,7,8,9,10-octahydropyrido[4′,3′:3,4]pyrazolo[1,5-a]pyrazine-7-carboxamide with (3R,7S)-2-(4-chloro-3-(trifluoromethyl)benzoyl)-9-(4-(cyclopropylsulfonyl)benzyl)-N,3-dimethyl-10-oxo-1,2,3,4,7,8,9,10-octahydropyrido[4′,3′:3,4]pyrazolo[1,5-a]pyrazine-7-carboxamide 20 (200 mg, 94% purity, 0.28 mmol) was separated by chiral prep HPLC (separation condition: Column: Chiralpak IB N-5 5 μm 20*250 mm; Mobile Phase: Hex:EtOH=30:70 at 25 mL/min; Temp: 30° C.; Wavelength: 254 nm) to give the title compounds 20A (47.5 mg, 99.7% purity, 25.2% yield, 99.9% stereopure), 20B (71.2 mg, 99.5% purity, 37.7% yield, 99.5% stereopure) and 21 (12.5 mg, 94.7% purity, 6.4% yield) as white solids.

20A

LC-MS (ESI): R_{T}=3.424 min, mass calcd. for C₃₁H₃₁ClF₃N₅O₅S 677.2, m/z found 678.2 [M+H]⁺. Chiral analysis (Column: Chiralpak IBN-5 5 μm 4.6*250 mm; Mobile Phase: Hex:EtOH=30:70 at 1 mL/min; Temp: 30° C.; Wavelength: 254 nm, R_{T}=6.532 min). ¹H NMR (400 MHz, DMSO-d₆) δ 7.93 (s, 1H), 7.86-7.85 (m, 3H), 7.80-7.79 (m, 2H), 7.57-7.44 (m, 2H), 5.94-5.68 (m, 1H), 5.50-5.19 (m, 1H), 5.05-4.93 (m, 1H), 4.70-4.35 (m, 1H), 4.29-3.93 (m, 2H), 3.44-3.34 (m, 1H), 2.98-2.81 (m, 2H), 2.67-2.53 (m, 1H), 2.36 (d, J=3.6 Hz, 3H), 1.63-1.43 (m, 3H), 1.30-1.17 (m, 3H), 1.12-1.11 (m, 2H), 1.04-1.02 (m, 2H). ¹⁹F NMR (376 MHz, DMSO-d₆) δ −61.31.

20B

LC-MS (ESI): R_{T}=3.459 min, mass calcd. for C₃₁H₃₁ClF₃N₅O₅S 677.2, m/z found 678.3 [M+H]⁺. Chiral analysis (Column: Chiralpak IBN-5 5 μm 4.6*250 mm; Mobile Phase: Hex:EtOH=30:70 at 1 mL/min; Temp: 30° C.; Wavelength: 254 nm, R_{T}=10.778 min). ¹H NMR (400 MHz, DMSO-d₆) δ 8.09-8.00 (m, 1H), 7.92 (s, 1H), 7.90-7.84 (m, 3H), 7.82-7.75 (m, 1H), 7.65-7.50 (m, 2H), 5.92-5.63 (m, 1H), 5.51-5.18 (m, 1H), 5.11-4.97 (m, 1H), 4.68-4.37 (m, 1H), 4.26-4.11 (m, 1H), 3.78-3.56 (m, 2H), 2.98-2.78 (m, 2H), 2.64 (d, J=4.8 Hz, 3H), 2.61-2.51 (m, 1H), 1.55-1.38 (m, 3H), 1.30-1.18 (m, 3H), 1.15-1.09 (m, 2H), 1.04-1.02 (m, 2H). ¹⁹F NMR (376 MHz, DMSO-d₆) δ −61.34.

21

LC-MS (ESI): R_{T}=3.553 min, mass calcd. for C₃₀H₂₉ClF₃N₅O₅S 663.2, m/z found 664.2 [M+H]⁺. ¹H NMR (400 MHz, DMSO-d₆) δ 7.98-7.96 (m, 1H), 7.92 (d, J=1.6 Hz, 1H), 7.86-7.79 (m, 4H), 7.57-7.41 (m, 2H), 5.52-5.18 (m, 1H), 5.12-4.91 (m, 2H), 4.65-4.32 (m, 2H), 4.26-4.04 (m, 2H), 3.81-3.63 (m, 1H), 2.94 (dd, J=16.4, 4.4 Hz, 1H), 2.87-2.80 (m, 1H), 2.64-2.51 (m, 4H), 1.30-1.15 (m, 3H), 1.14-1.08 (m, 2H), 1.07-0.97 (m, 2H). ¹⁹F NMR (376 MHz, DMSO-d₆) δ −61.33.

Compounds 22A and 22B

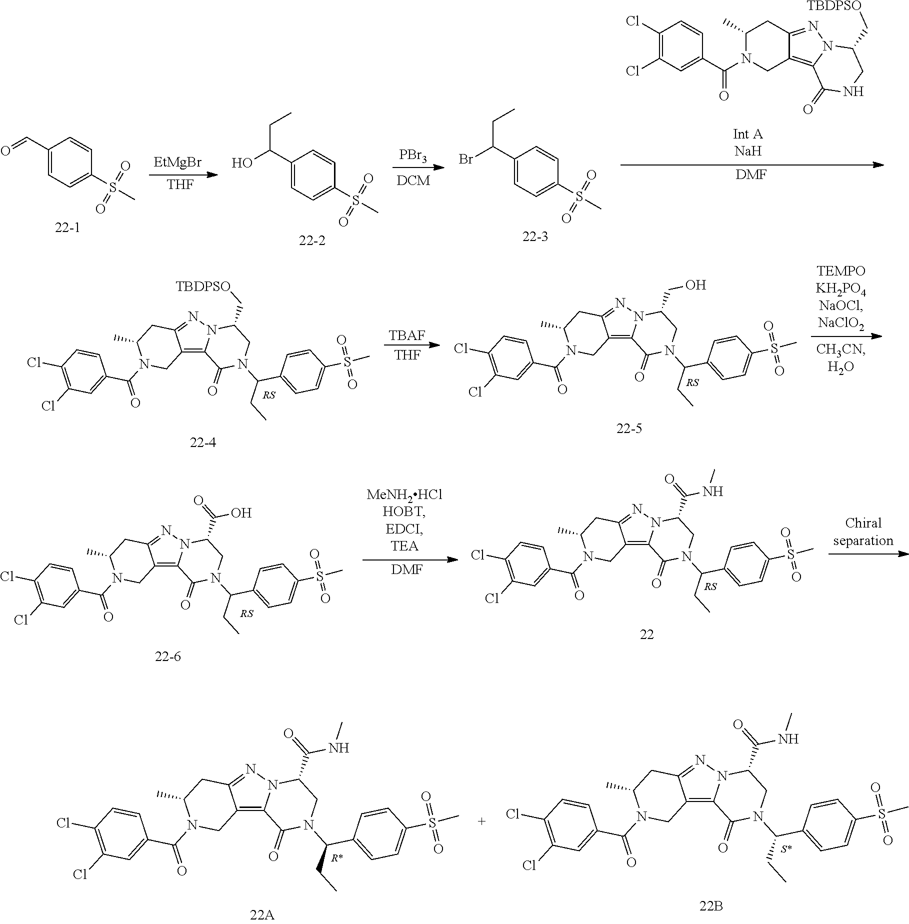

Intermediate 22-2

1-(4-(Methylsulfonyl)phenyl)propan-1-ol

To a solution of 4-(methylsulfonyl)benzaldehyde 22-1 (3.0 g, 16.2 mmol) in tetrahydrofuran (50 mL) was added 2.0 M ethylmagnesium bromide in tetrahydrofuran (12 mL, 24 mmol) at −78° C. under nitrogen atmosphere. The resulting mixture was stirred for 2 hours, the reaction mixture was quenched with saturated ammonium chloride aqueous solution (100 mL), extracted with ethyl acetate (50 mL) for three times, dried over Na₂SO₄₍ₛ₎ and filtered. The filtrate was concentrated to get a residue, which was purified by C18 chromatography (acetonitrile:water (+0.02% ammonium bicarbonate)=15-30%) to give the title compound (2.0 g, 90% purity from ¹H NMR, 51.5% yield) as white solids. ¹H NMR (400 MHz, CDCl₃) δ 7.93 (d, J=8.4 Hz, 2H), 7.56 (d, J=8.0 Hz, 2H), 4.76-4.72 (m, 1H), 3.05 (s, 3H), 1.99-1.97 (m, 1H), 1.84-1.75 (m, 2H), 0.95 (d, J=8.0 Hz, 3H).

Intermediate 22-3

1-(1-Bromopropyl)-4-(methylsulfonyl)benzene

To a solution of 1-(4-(methylsulfonyl)phenyl)propan-1-ol 22-2 (1.5 g, 90% purity, 6.3 mmol) in dichloromethane (40 mL) was added phosphorus(III) bromide (3 g, 11.1 mmol) at 0° C. under nitrogen atmosphere. After being stirred for 1 hour at room temperature, the reaction mixture was concentrated under reduced pressure to get a crude, the crude was diluted with ethyl acetate (50 mL) then washed with saturated sodium bicarbonate solution (50 mL) twice and dried over Na₂SO₄₍ₛ₎ and filtered. The filtrate was concentrated under reduced pressure to give title compound (600 mg, 90% purity from 1H NMR, 31% yield) as yellow oil. ¹H NMR (400 MHz, CDCl₃) δ 7.93 (d, J=8.4 Hz, 2H), 7.59 (d, J=8.4 Hz, 2H), 4.88 (t, J=6.9 Hz, 1H), 3.07 (s, 3H), 2.35-2.11 (m, 2H), 1.02 (t, J=7.2 Hz, 3H).

Intermediate 22-4

(3R,7S)-7-(((tert-butyldiphenylsilyl)oxy)Methyl)-2-(3,4-dichlorobenzoyl)-3-methyl-9-(1-(4-(methylsulfonyl)phenyl)propyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (800 mg, 89% purity, 1.10 mmol) in N,N-dimethylformamide (20 mL) was added 60% sodium hydride in mineral oil (200 mg, 4.58 mmol). After being stirred at 0° C. for 30 minutes, 1-(1-bromopropyl)-4-(methylsulfonyl)benzene 22-3 (600 mg, 90% purity, 1.95 mmol) was added to the mixture. After being stirred at 0° C. for 1 hour, the reaction mixture was quenched with water (50 mL) and extracted with ethyl acetate (50 mL) twice. The combined organic layers were washed with brine (100 mL) twice, dried over Na₂SO₄₍ₛ₎, filtered and concentrated under reduced pressure to give crude mixture compounds (1 g, 83% purity from LCMS, 89% yield) as yellow solids. LC-MS (ESI): R_{T}=1.45 min, mass calcd. for C₄₄H₄₈Cl₂N₄O₅SSi 842.25, m/z found 605.4 [M−OTBDPS]⁺.

Intermediate 22-5

(3R,7S)-2-(3,4-Dichlorobenzoyl)-7-(hydroxymethyl)-3-methyl-9-(1-(4-(methylsulfonyl)phenyl)propyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-9-(1-(4-(methylsulfonyl)phenyl)propyl)-1,2,3,4,8,9-hexahydropyrido[4′,3′:3,4]pyrazolo[1,5-a]pyrazin-10(7H)-one 22-4 (1 g, 83% purity, 0.983 mmol) in tetrahydrofuran (10 mL) was added 1 M tetrabutylammonium fluoride in tetrahydrofuran (1.5 mL, 1.5 mmol) at 0° C. After being stirred at 0° C. for 1 hour, the reaction mixture was concentrated under reduced pressure to give a crude, which was purified by silica gel column chromatography (dichloromethane:ethyl acetate=5:1 to 3:1) to give the title compound (400 mg, 86% purity from LCMS, 58% yield) as white solids. LC-MS (ESI): R_{T}=1.51 min, mass calcd. for C₂₈H₃₀Cl₂N₄O₅S 604.13, m/z found 605.0 [M+H]⁺.

Intermediate 22-6

(3R,7S)-2-(3,4-Dichlorobenzoyl)-3-methyl-9-(1-(4-(methylsulfonyl)phenyl)propyl)-10-oxo-1,2,3,4,7,8,9,10-octahydropyrido[4′,3′:3,4]pyrazolo[1,5-a]pyrazine-7-carboxylic acid

To a suspension of (3R,7S)-2-(3,4-dichlorobenzoyl)-7-(hydroxymethyl)-3-methyl-9-(1-(4-(methylsulfonyl)phenyl)propyl)-1,2,3,4,8,9-hexahydropyrido[4′,3′:3,4] pyrazolo[1,5-a]pyrazin-10(7H)-one 22-5 (400 mg, 86% purity, 0.568 mmol) in acetonitrile (10 mL) and saturated potassium dihydrogen phosphate aqueous solution (10 mL) was added 2,2,6,6-tetramethylpiperidinyloxy (200 mg, 1.28 mmol), sodium chlorite (140 mg, 1.24 mmol) and sodium hypochlorite aqueous solution (0.8 mL, 1.34 mmol) was added at 0° C. After being stirred at room temperature for 1 hour, the reaction mixture was adjusted PH to 5-6 by 1M hydrochloride aqueous solution and extracted with ethyl acetate (50 mL) twice. The combined organic layers were washed with brine (50 mL), dried over Na₂SO₄, filtered and concentrated under reduced pressure to give crude compound (400 mg, 87% purity from LCMS, 99% yield) as yellow solids. LC-MS (ESI): R_{T}=1.25 and 1.27 mins, mass calcd. for C₂₈H₂₈Cl₂N₄O₆S 618.11, m/z found 619.8 [M+H]⁺.

Compound 22

(3R,7S)-2-(3,4-Dichlorobenzoyl)-N,3-dimethyl-9-(1-(4-(methylsulfonyl)phenyl) propyl)-10-oxo-1,2,3,4,7,8,9,10-octahydropyrido[4′,3′:3,4]pyrazolo[1,5-a]pyrazine-7-carboxamide

To a solution of (3R,7S)-2-(3,4-dichlorobenzoyl)-3-methyl-9-(1-(4-(methylsulfonyl)phenyl)propyl)-10-oxo-1,2,3,4,7,8,9,10-octahydropyrido[4′,3′:3,4] pyrazolo [1,5-a]pyrazine-7-carboxylic acid 22-6 (400 mg, 87% purity, 0.562 mmol), methanamine hydrochloride (130 mg, 1.93 mmol), 1-Hydroxybenzotriazole (200 mg, 1.48 mmol) and 1-(3-dimethylaminopropyl)-3-ethylcarbodiimide hydrochloride (300 mg, 1.57 mmol) in N,N-dimethylformamide (10 mL) was added dropwise triethylamine (0.7 mL, 5.04 mmol) at 0° C. After being stirred for 1 hour at 0° C., the mixture was diluted with water (50 mL) and extracted with ethyl acetate (50 mL) twice. The combined organic layers were dried over Na₂SO₄₍ₛ₎ and filtered. The filtrate was concentrated under reduced pressure to give the crude, which was purified by silica gel column chromatography (dichloromethane:acetone=20:1 to 5:1) to give compound (250 mg, 73% purity from LCMS, 51% yield) as white solids. LC-MS (ESI): R_{T}=1.48 min, mass calcd. for C₂₉H₃₁Cl₂N₅O₅S 631.14, m/z found 648.9 [M+H₂O]⁺.

Compounds 22A and 22B

(3R,7S)-2-(3,4-Dichlorobenzoyl)-N,3-dimethyl-9-((R*)-1-(4-(methylsulfonyl)phen yl)propyl)-10-oxo-1,2,3,4,7,8,9,10-octahydropyrido[4′,3′:3,4]pyrazolo[1,5-a]pyrazine-7-carboxamide (22A), and (3R,7S)-2-(3,4-dichlorobenzoyl)-N,3-dimethyl-9-((S*)-1-(4-(methylsulfonyl)phen yl)propyl)-10-oxo-1,2,3,4,7,8,9,10-octahydropyrido[4′,3′:3,4]pyrazolo[1,5-a]pyrazine-7-carboxamide (22B)

A racemic mixture of (3R,7S)-2-(3,4-dichlorobenzoyl)-N,3-dimethyl-9-(1-(4-(methylsulfonyl)phenyl)propyl)-10-oxo-1,2,3,4,7,8,9,10-octahydropyrido[4′,3′:3,4]pyrazolo[1,5-a]pyrazine-7-carboxamide 22 (250 mg, 73% purity, 0.289 mmol) was separated by chiral prep. HPLC (separation condition: Column: Chiralpak IB N-5 250 mm*20 mm 5 μm; Mobile Phase: ACN:IPA=90:10 at 15 mL/min; Temp: 30° C.; Wavelength: 254 nm), then further purified by Prep. HPLC (Column: Xbridge C18 (5 μm 19*150 mm), Mobile Phase A: Water (+0.1% ammonium bicarbonate), Mobile Phase B: acetonitrile, UV: 214 nm, Flow rate: 15 mL/min, Gradient: 30-60% (% B)), to give the title compounds 22A (23.9 mg, 99.7% purity, 54% yield, 99.9% stereopure) as white solids and 22B (46.3 mg, 99.8% purity, 60% yield, 99.5% stereopure) as white solids.

22A

LC-MS (ESI): R_{T}=3.349 min, mass calcd. for C₂₉H₃₁Cl₂N₅O₅S 631.14 m/z found 632.2 [M+H]⁺. Chiral analysis (Chiralpak IB N-5 250 mm*4.6 mm 5 μm: ACN:IPA=90:10 at 1 mL/min; Temp: 30° C.; Wavelength: 254 nm, R_{T}=4.214 min). ¹H NMR (400 MHz, CDCl₃) δ 7.91 (d, J=8.0 Hz, 2H), 7.54-7.51 (m, 4H), 7.30-7.27 (m, 111), 6.09-5.40 (m, 3H), 4.93-4.45 (m, 3H), 3.90 (d, J=4.0 Hz, 2H), 3.05 (s, 3H), 3.01-2.95 (m, 1H), 2.73-2.69 (m, 1H), 2.64 (d, J=4.8 Hz, 3H), 2.14-2.00 (m, 2H), 1.29 (d, J=6.8 Hz, 3H), 1.01 (t, J=6.4 Hz, 3H).

22B

LC-MS (ESI): R_{T}=3.404 min, mass calcd. for C₂₉H₃₁Cl₂N₅O₅S 631.14 m/z found 632.2 [M+H]⁺. Chiral analysis (Chiralpak IB N-5 250 mm*4.6 mm 5 μm: ACN:IPA=90:10 at 1 mL/min; Temp: 30° C.; Wavelength: 254 nm, R_{T}=5.754 min). 11H NMR (400 MHz, CDCl₃) δ 7.93 (d, J=8.4 Hz, 2H), 7.62-7.57 (m, 2H), 7.54-7.52 (m, 2H), 7.29-7.27 (m, 111), 5.96-5.38 (m, 3H), 4.84-4.38 (m, 3H), 4.14-4.10 (m, 111), 3.36-3.31 (m, 111), 3.05 (s, 3H), 3.02-2.96 (m, 1H), 2.80 (d, J=4.8 Hz, 3H), 2.73-2.69 (m, 1H), 2.12-2.04 (m, 2H), 1.32 (d, J=7.2 Hz, 3H), 0.95 (t, J=7.2 Hz, 3H).

Compounds 23A and 23B

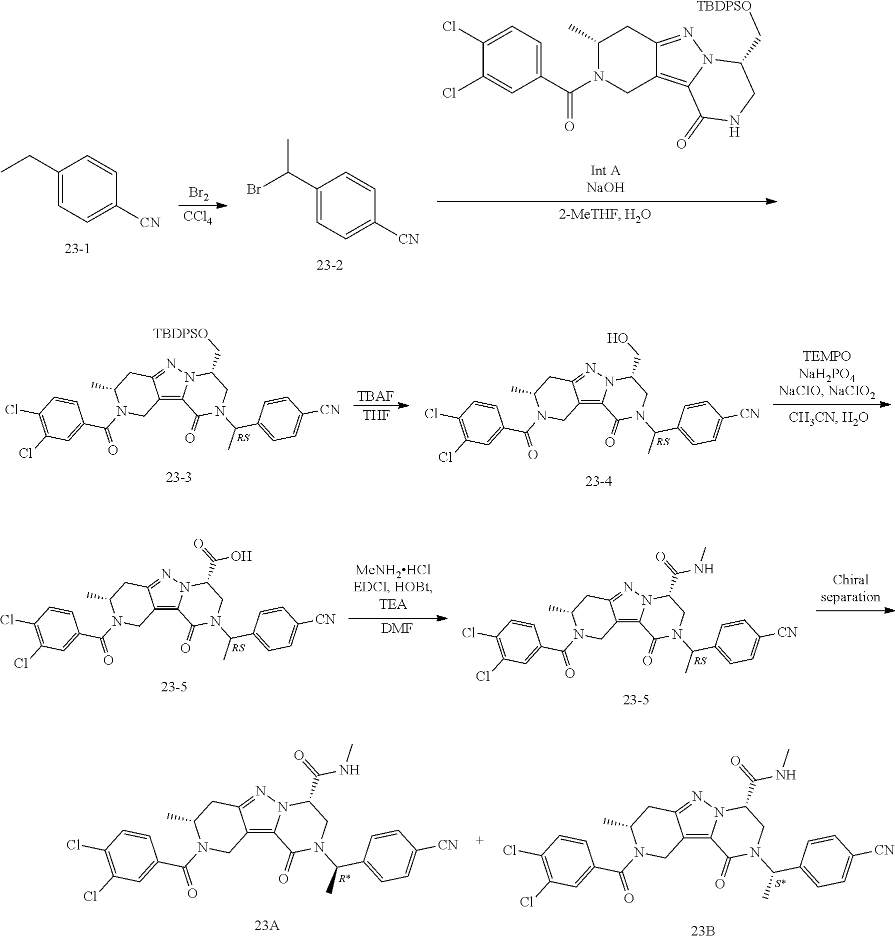

Intermediate 23-2

4-(1-Bromoethyl)benzonitrile

A solution of 4-ethylbenzonitrile 23-1 (1.2 g, 9.1 mmol) in carbon tetrachloride (5.0 mL) was heated to reflux. Then bromine (1.5 g, 9.39 mmol) was added slowly over 30 minutes under UV irradiation. The reaction mixture was refluxed for 4 hours and cooled to room temperature. Then the mixture was diluted with dichloromethane (50 mL) and washed with saturated sodium bicarbonate aqueous solution (50 mL) and brine (50 mL). The combined organic layers were dried over Na₂SO₄₍ₛ₎ and the solvents were removed under reduced pressure to give the title product (2.0 g, 60% purity from 1H NMR, 62% yield) as a brown liquid. ¹H NMR (400 MHz, CDCl₃) δ 7.65-7.63 (m, 2H), 7.55-7.53 (m, 2H), 5.16 (q, J=7.2 Hz, 1H), 2.04 (d, J=7.2 Hz, 31H).

Intermediate 23-3

4-(1-((3R,7S)-7-(((tert-Butyldiphenylsilyl)oxy)methyl)-2-(3,4-dichlorobenzoyl)-3-methyl-10-oxo-1,2,3,4,7,8-hexahydropyrido[4′,3′:3,4]pyrazolo[1,5-a]pyrazin-9(10H)-yl)ethyl)benzonitrile

To a solution of (3R,7S)-7-(((tert-butyldiphenylsilyl)oxy)methyl)-2-(3,4-di chlorobenzoyl)-3-methyl-1,2,3,4,8,9-hexahydropyrido[4′,3′:3,4]pyrazolo[1,5-a]pyrazin-10(7H)-one Int A (1.0 g, 90% purity, 1.39 mmol) in 2-methyl tetrahydrofuran (10 mL) was added benzyltriethylammonium chloride (65 mg, 0.285 mmol), 4-(1-bromoethyl)benzonitrile 23-2 (1.3 g, 60% purity, 3.71 mmol) and 50% wt. sodium hydroxide in water (10 mL) at 0° C. After being stirred at room temperature for 6 hours, the mixture was diluted with water (200 mL), extracted with ethyl acetate (100 mL) for three times. The combined organic layers were washed with brine (250 mL), dried over Na₂SO₄₍ₛ₎ and filtered. The filtrate was concentrated to give the crude compound (1.9 g, 45% purity from LCMS, 79% yield) as yellow oil. LC-MS (ESI): R_{T}=4.30 min and 4.40 min, mass calcd. for C₄₃H₄₃Cl₂N₅O₃Si 775.3, m/z found 776.1 [M+H]⁺.

Intermediate 23-4

4-(1-((3R,7S)-2-(3,4-Dichlorobenzoyl)-7-(hydroxymethyl)-3-methyl-10-oxo-1,2,3,4,7,8-hexahydropyrido[4′,3′:3,4]pyrazolo[1,5-a]pyrazin-9(10H)-yl)ethyl)benzonitrile

To a solution of 4-(1-((3R,7S)-7-(((tert-butyldiphenylsilyl)oxy)methyl)-2-(3,4-dichlorobenzoyl)-3-methyl-10-oxo-1,2,3,4,7,8-hexahydropyrido[4′,3′:3,4]pyrazolo[1,5-a]pyrazin-9(10H)-yl)ethyl)benzonitrile 23-3 (1.9 g, 45% purity, 1.10 mmol) in tetrahydrofuran (15 mL) was added 1 M tetrabutylammonium fluoride in tetrahydrofuran (2.5 mL, 2.5 mmol) at 0° C. After being stirred at 0° C. for 1 hour and then at room temperature for 1 hour, the mixture was quenched with saturated ammonium chloride (80 mL), extracted with ethyl acetate (100 mL) for three times. The combined organic layers were washed with brine (100 mL), dried over Na₂SO₄₍ₛ₎, filtered. The filtrate was concentrated and purified by silica gel column chromatography (ethyl acetate: petroleum ether=1:1 to 5:1) to give the title compound (628 mg, 72% purity from LCMS, 76% yield) as yellow solids. LC-MS (ESI): R_{T}=2.81 min and 2.83 min, mass calcd. for C₂₇H₂₅Cl₂N₅O₃ 537.1, m/z found 538.1 [M+H]⁺.

Intermediate 23-5

(3R,7S)-9-(1-(4-Cyanophenyl)ethyl)-2-(3,4-dichlorobenzoyl)-3-methyl-10-oxo-1,2,3,4,7,8,9,10-octahydropyrido[4′,3′:3,4]pyrazolo[1,5-a]pyrazine-7-carboxylic acid

To a solution of 4-(1-((3R,7S)-2-(3,4-dichlorobenzoyl)-7-(hydroxymethyl)-3-methyl-10-oxo-1,2,3,4,7,8-hexahydropyrido[4′,3′:3,4]pyrazolo[1,5-a]pyrazin-9(10H)-yl)ethyl)benzonitrile 23-4 (307 mg, 72% purity, 0.411 mmol) in acetonitrile (3 mL) was added saturated potassium dihydrogen phosphate aqueous solution (3 mL), 2,2,6,6-tetramethylpiperidinooxy (150 mg, 0.960 mmol), sodium chlorite (120 mg, 1.061 mmol) and dropwise 5.5% sodium hypochlorite aqueous solution (0.6 mL, 1.01 mmol) at 0° C. The reaction was allowed to slowly cool down to room temperature. After being stirred at room temperature for 4 hours, the reaction mixture was quenched with saturated sodium thiosulfate (8 mL), acidified with 1 M hydrochloride aqueous solution (about 4 mL) to pH 3-4, extracted with ethyl acetate (10 mL) for three times. The combined organic layers were washed with brine (10 mL), dried over Na₂SO₄₍ₛ₎, filtered. The filtrate was concentrated and purified by triturated with petroleum ether:ethyl acetate=5:1 (10 mL) at room temperature. After being stirred for 30 minutes, the mixture was filtered to give the title compound (225 mg, 90% purity from LCMS, 89% yield) as white solids. LC-MS (ESI): R_{T}=2.19 min and 2.22 min, mass calcd. for C₂₇H₂₃Cl₂N₅O₄ 551.1, m/z found 552.1 [M+H]⁺.

Compound 23

(3R,7S)-9-(1-(4-Cyanophenyl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide

To a solution of (3R,7S)-9-(1-(4-cyanophenyl)ethyl)-2-(3,4-dichlorobenzoyl)-3-methyl-10-oxo-1,2,3,4,7,8,9,10-octahydropyrido[4′,3′:3,4]pyrazolo[1,5-a]pyrazine-7-carboxylic acid 23-5 (225 mg, 90% purity, 0.367 mmol) in N,N-dimethylformamide (5 mL) was added 1-(3-dimethylaminopropyl)-3-ethyl carbodiimide hydrochloride (150 mg, 0.782 mmol), 1-hydroxybenzotriazole (100 mg, 0.740 mmol), methanamine hydrochloride (75 mg, 1.11 mmol) and dropwise triethylamine (0.4 mL, 2.88 mmol) in N,N-dimethylformamide (5 mL) at 0° C.

After being stirred at 0° C. for 10 minutes, the mixture was added water (50 mL), extracted with ethyl acetate (40 mL) for three times. The organic layers were washed with brine (20 mL), dried over Na₂SO₄₍ₛ₎, filtered. The filtrate was concentrated and purified by C18 column (acetonitrile:water=5% to 65%) to give the title compound (190 mg, 94% purity from LCMS, 86% yield) as white solids. LC-MS (ESI): R_{T}=2.78 min and 2.82 min, mass calcd. for C₂₈H₂₆Cl₂N₆O₃ 564.1, m/z found 565.1 [M+H]⁺.

Compounds 23A and 23B

(3R,7S)-9-((R*)-1-(4-Cyanophenyl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide (23A), and (3R,7S)-9-((S*)-1-(4-cyanophenyl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide (23B)

A racemic mixture of (3R,7S)-2-(3,4-dichlorobenzoyl)-9-(1-(6-methoxypyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide 23 (325 mg, 99% purity, 0.569 mmol) was purified by chiral HPLC (separation condition: Column: Chiralpak IA 5 μm 30*250 mm; Mobile Phase: Hex:EtOH=30:70 at 25 mL/min; Temp: 30° C.; Wavelength: 254 nm) to get the title compounds 23A (100 mg, 99.2% purity, 31% yield, 100% stereopure) as white solids and 23B (130 mg, 98.5% purity, 40% yield, 100% stereopure) as white solids.

23A

LC-MS (ESI): R_{T}=3.714 min, mass calcd. for C₂₈H₂₆Cl₂N₆O₃ 564.1, m/z found 565.2 [M+H]⁺. Chiral analysis (Column: Chiralpak IA 5 μm 4.6*250 mm; Mobile Phase: Hex:EtOH=30:70 at 1 mL/min; Temp: 30° C.; Wavelength: 254 nm, R_{T}=7.044 min). ¹H NMR (400 MHz, DMSO-d₆) δ 7.90-7.76 (m, 5H), 7.53-7.35 (m, 3H), 5.89-5.64 (m, 1H), 5.47-5.11 (m, 1H), 5.00 (s, 1H), 4.66-3.92 (m, 3H), 3.41-3.36 (m, 1H), 2.98-2.82 (m, 1H), 2.64-2.52 (m, 1H), 2.40 (d, J=4.0 Hz, 3H), 1.60-1.40 (m, 3H), 1.29-1.11 (m, 3H).

23B

LC-MS (ESI): R_{T}=3.763 min, mass calcd. for C₂₈H₂₆Cl₂N₆O₃ 564.1, m/z found 565.2 [M+H]⁺. Chiral analysis (Column: Chiralpak IA 5 μm 4.6*250 mm; Mobile Phase: Hex:EtOH=30:70 at 1 mL/min; Temp: 30° C.; Wavelength: 254 nm, R_{T}=9.973 min). ¹H NMR (400 MHz, DMSO-d₆) δ 8.03 (s, 1H), 7.89-7.70 (m, 4H), 7.59-7.38 (m, 3H), 5.89-5.60 (m, 1H), 5.49-5.14 (m, 1H), 5.04 (s, 1H), 4.65-4.04 (m, 2H), 3.81-3.54 (m, 2H), 2.97-2.83 (m, 1H), 2.63 (d, J=4.8 Hz, 3H), 2.60-2.55 (m, 1H), 1.46 (s, 3H), 1.26-1.11 (m, 3H).

Compounds 24A and 24B

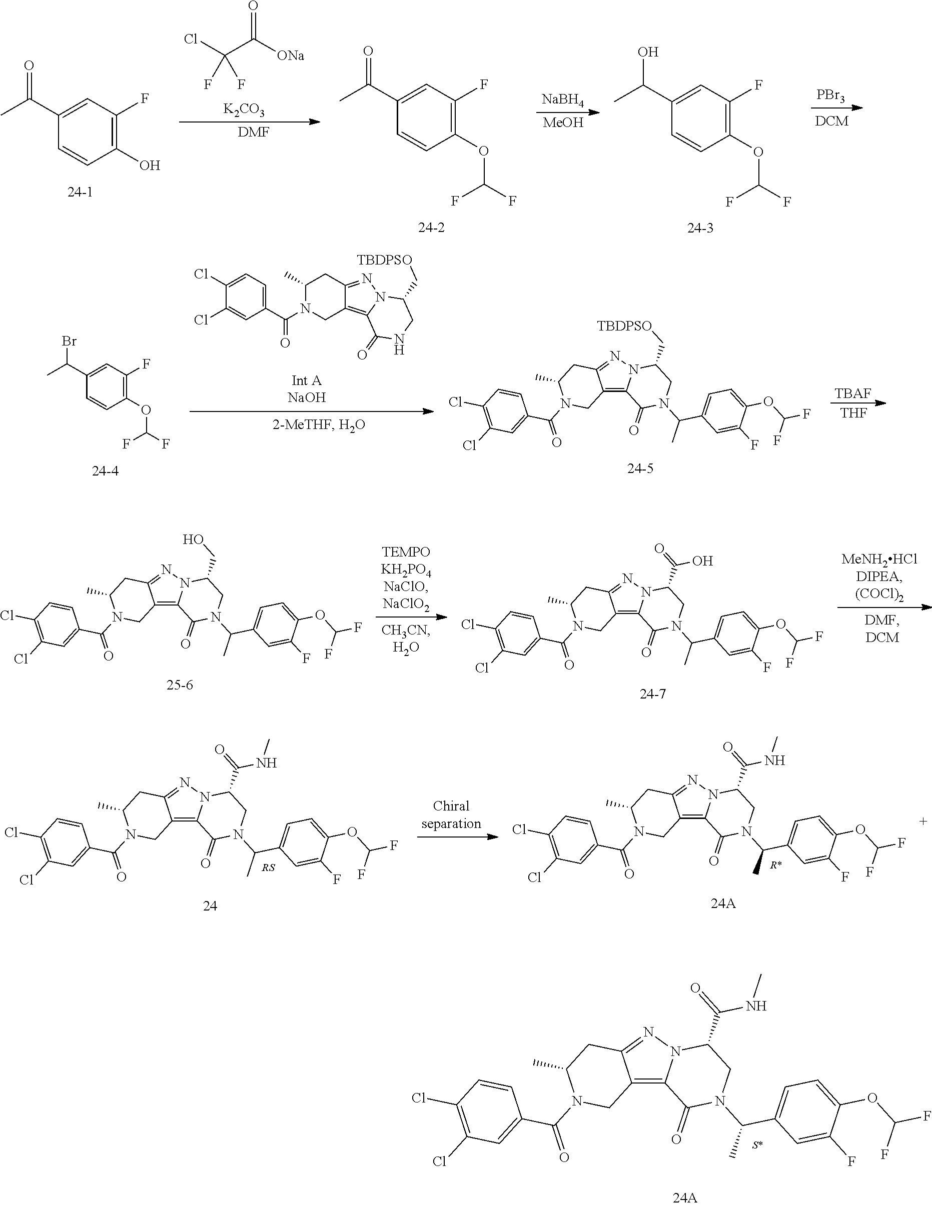

Intermediate 24-2

1-(4-(Difluoromethoxy)-3-fluorophenyl)ethanone

A mixture of 1-(3-fluoro-4-hydroxyphenyl)ethanone 24-1 (1.5 g, 9.73 mmol) N,N-dimethylformamide (15 mL) and potassium carbonate (1.6 g, 11.6 mmol) was stirred for 10 minutes under nitrogen atmosphere. Sodium 2-chloro-2,2-difluoroacetate (1.8 g, 11.8 mmol) was added and the mixture was heated to 100° C. overnight. The mixture was cooled to room temperature and treated with water (30 mL). The reaction mixture was extracted with ethyl acetate (30 mL) for three times, dried over Na₂SO₄₍ₛ₎, filtered and concentrated. The residue was purified by column chromatography on silica gel (petroleum ether:ethyl acetate=10:1) to give the title compound (1.3 g, 100% purity from ¹H NMR, 65.4% yield) as colorless oil. ¹H NMR (300 MHz, CDCl₃) δ 7.84-7.78 (m, 2H), 7.37-7.31 (m, 1H), 6.68 (t, J=72.6 Hz, 1H), 2.64 (s, 3H).

Intermediate 24-3

1-(4-(Difluoromethoxy)-3-fluorophenyl)ethanol

To a solution of 1-(4-(difluoromethoxy)-3-fluorophenyl)ethanone 24-2 (1.3 g, 6.37 mmol) in methanol (15 mL) was added sodium borohydride (150 mg, 3.97 mmol) at 0° C., then the mixture was stirred for 2 hours. The solution was quenched with water (30 mL) and extracted with ethyl acetate (50 mL) for three times, washed with brine (30 mL), dried over Na₂SO₄₍ₛ₎ and filtered. The filtrate was concentrated to give the title product (1.3 g, 100% purity from ¹H NMR, 99.0% yield) as colorless oil. ¹H NMR (300 MHz, CDCl₃) δ 7.31-7.23 (m, 2H), 7.16 (d, J=8.7 Hz, 1H), 6.58 (t, J=73.5 Hz, 1H), 4.93 (q, J=6.3 Hz, 1H), 1.99 (s, 1H), 1.52 (d, J=6.3 Hz, 3H).

Intermediate 24-4

4-(1-Bromoethyl)-1-(difluoromethoxy)-2-fluorobenzene

To a solution of 1-(4-(1-methyl-1H-pyrazol-3-yl)phenyl)ethanol 24-3 (600 mg, 100% purity, 2.91 mmol) in dichloromethane (6 mL) was added dropwise tribromophosphine (600 mg, 2.22 mmol) in dichloromethane (4 mL) at 0° C. After being stirred at 0° C. for 3 hours, the reaction was poured into water (10 mL), extracted with dichloromethane (10 mL) for three times. The combined organic layers were washed with brine (10 mL), dried over Na₂SO₄₍ₛ₎, and filtered. The filtrate was concentrated under reduced pressure to give the title product (700 mg, 90% purity from ¹H NMR, 80.5% yield) as yellow oil. ¹H NMR (300 MHz, CDCl₃) δ 7.35-7.31 (m, 2H), 7.26-7.24 (m, 1H), 6.60 (t, J=73.5 Hz, 1H), 5.18 (q, J=6.9 Hz, 1H), 2.23 (d, J=6.9 Hz, 3H).

Intermediate 24-5

(3R,7S)-7-(((tert-Butyldiphenylsilyl)oxy)methyl)-2-(3,4-dichlorobenzoyl)-9-(1-(4-(difluoromethoxy)-3-fluorophenyl)ethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (750 mg, 100% yield, 1.16 mmol) and 4-(1-bromoethyl)-1-(difluoromethoxy)-2-fluorobenzene 24-4 (700 mg, 90% purity, 2.34 mmol) in 2-methyltetrahydrofuran (6 mL) were added sodium hydroxide in water (3 mL, 50% wt) and benzyltriethylammonium chloride (30 mg, 0.13 mmol) at 25° C. After being stirred at 50° C. for 2 hours, the reaction was poured into water (10 mL), extracted with ethyl acetate (10 mL) for three times. The combined organic layers were washed with brine (10 mL), dried over Na₂SO₄₍ₛ₎, and filtered. The filtrate was concentrated in under reduced pressure to give the crude product (1.6 g, 58% purity from LCMS, 95.9% yield) as yellow oil. The crude was used for next step without further purification. LC-MS (ESI): R_{T}=2.36 min, mass calcd. for C₄₃H₄₃Cl₂F₃N₄O₄Si 834.2, m/z found 835.4 [M+H]⁺.

Intermediate 24-6

(3R,7S)-2-(3,4-Dichlorobenzoyl)-9-(1-(4-(difluoromethoxy)-3-fluorophenyl)ethyl)-7-(hydroxymethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-9-(1-(4-(difluoromethoxy)-3-fluorophenyl)ethyl)-3-methyl-1,2,3,4,8,9-hexahydropyrido[4′,3′:3,4]pyrazolo[1,5-a]pyrazin-10(7H)-one 24-5 (1.6 g, 58% purity, 1.11 mmol) in tetrahydrofuran (20 mL) was added 1 M tetrabutylammonium fluoride solution in tetrahydrofuran (1.2 mL, 1.20 mmol) at 0° C. After being stirred at 20° C. for 2 hours, the reaction was poured into water (20 mL), extracted with ethyl acetate (50 mL) for three times. The combined organic layers were washed with brine (20 mL), dried over Na₂SO₄₍ₛ₎, and filtered. The filtrate was concentrated in vacuum, then was purified by silica gel column chromatography (petroleum ether:ethyl acetate=5:1 to 2:1) to give the title product (640 mg, 91% purity from LCMS, 87.8% yield) as colorless solids. LC-MS (ESI): R_{T}=1.67 min, mass calcd. for C₂₇H₂₅Cl₂F₃N₄O₄ 596.1 m/z found 597.1 [M+H]⁺. ¹H NMR (300 MHz, CDCl₃) δ 7.59-7.54 (m, 2H), 7.37-7.32 (m, 1H), 7.30-7.29 (m, 1H), 7.28-7.26 (m, 2H), 6.60 (t, J=72.9 Hz, 1H), 6.08 (s, 1H), 5.89-5.24 (m, 1H), 5.09-4.22 (m, 3H), 4.09-3.99 (m, 1H), 3.95-3.74 (m, 1H), 3.68-3.50 (m, 1H), 3.36-3.19 (m, 1H), 3.15-2.96 (m, 1H), 2.76-2.73 (m, 1H), 2.68-2.57 (m, 1H) 1.62-1.57 (m, 3H), 1.31 (d, J=5.7 Hz, 3H).

Intermediate 24-7

(3R,7S)-2-(3,4-Dichlorobenzoyl)-9-(1-(4-(difluoromethoxy)-3-fluorophenyl)ethyl)-3-methyl-10-oxo-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-9-(1-(4-(difluoromethoxy)-3-fluorophenyl)ethyl)-7-(hydroxymethyl)-3-methyl-1,2,3,4,8,9-hexahydropyrido[4′,3′:3,4]pyrazolo[1,5-a]pyrazin-10(7H)-one 24-6 (300 mg, 91% purity, 0.46 mmol) in acetonitrile (3 mL) were added saturated potassium dihydrogen phosphate aqueous solution (3 mL), 2,2,6,6-tetramethylpiperidinooxy (150 mg, 0.96 mmol), sodium chlorite (85 mg, 0.94 mmol) and dropwise 10% sodium hypochlorite aqueous solution (700 mg, 0.94 mmol) at 0° C. The reaction was allowed to slowly return to room temperature. After being stirred at room temperature for 4 hours, the reaction mixture was quenched with saturated sodium thiosulfate (7 mL), acidized with 1 M hydrochloride aqueous solution to pH=4-5, extracted with ethyl acetate (10 mL) for three times. The combined organic layers were washed with brine (10 mL), dried over Na₂SO₄₍ₛ₎, filtered. The filtrate was concentrated and purified by C18 column (acetonitrile:water=5% to 40%) to give the title compound (210 mg, 100% purity from LCMS, 75.2% yield) as white solids. LC-MS (ESI): R_{T}=1.35 min, mass calcd. for C₂₇H₂₃Cl₂F₃N₄O₅ 610.1, m/z found 611.1 [M+H]⁺.

Compound 24

(3R,7S)-2-(3,4-Dichlorobenzoyl)-9-(1-(4-(difluoromethoxy)-3-fluorophenyl)ethyl)-N,3-dimethyl-10-oxo-1,2,3,4,7,8,9,10-octahydropyrido[4′,3′:3,4]pyrazolo[1,5-a]pyrazine-7-carboxamide

To a solution of (3R,7S)-2-(3,4-dichlorobenzoyl)-9-(1-(4-(difluoromethoxy)-3-fluorophenyl)ethyl)-3-methyl-10-oxo-1,2,3,4,7,8,9,10-octahydropyrido[4′,3′:3,4]pyrazolo[1,5-a]pyrazine-7-carboxylic acid 24-7 (210 mg, 100% purity, 0.34 mmol) in dichloromethane (3 mL) was added oxalyl dichloride (130 mg, 1.02 mmol) dropwise and N,N-dimethylformamide (10 mg, 0.14 mmol) at 0° C. After being stirred at 20° C. for 1 hour, the mixture was cooled to 0° C. and 2 M methylamine solution in tetrahydrofuran (1 mL, 2.00 mmol), N-ethyl-N-isopropylpropan-2-amine (150 mg, 1.16 mmol) were added dropwise at 0° C. The resulting mixture was stirred at 20° C. for 3 hours, then poured into water (10 mL), and extracted with dichloromethane (10 mL) for three times. The combined organic layers were washed with brine (10 mL), dried over Na₂SO₄₍ₛ₎, and filtered. The filtrate was concentrated in vacuum to give a residue, which was purified by C18 chromatography (acetonitrile:water (+0.02% ammonium bicarbonate)=40-60%) to give the title compound (190 mg, 100% purity from LCMS, 88.6% yield) as colorless solids. LC-MS (ESI): R_{T}=1.63 min, mass calcd. for C₂₈H₂₆Cl₂F₃N₅O₄ 623.1, m/z found 624.1 [M+H]⁺.

Compounds 24A and 24B

(3R,7S)-2-(3,4-Dichlorobenzoyl)-9-((R*)-1-(4-(difluoromethoxy)-3-fluorophenyl)ethyl)-N,3-dimethyl-10-oxo-1,2,3,4,7,8,9,10-octahydropyrido[4′,3′:3,4]pyrazolo[1,5-a]pyrazine-7-carboxamide (24A), and (3R,7S)-2-(3,4-dichlorobenzoyl)-9-((S*)-1-(4-(difluoromethoxy)-3-fluorophenyl)ethyl)-N,3-dimethyl-10-oxo-1,2,3,4,7,8,9,10-octahydropyrido[4′,3′:3,4]pyrazolo[1,5-a]pyrazine-7-carboxamide (24B)

The racemate of (3R,7S)-2-(3,4-dichlorobenzoyl)-9-(1-(4-(difluoromethoxy)-3-fluorophenyl)ethyl)-N,3-dimethyl-10-oxo-1,2,3,4,7,8,9,10-octahydropyrido[4′,3′:3,4]pyrazolo[1,5-a]pyrazine-7-carboxamide 24 (190 mg, 100% purity, 0.25 mmol) was separated by chiral prep HPLC (separation condition: Column: Chiralpak IE 5 μm 20*250 mm; Mobile Phase: Hex:EtOH=40:60 at 25 mL/min; Temp: 30° C.; Wavelength: 254 nm) to give the title compound 24A (50.3 mg, 99.4% purity, 26.3% yield, 100% stereopure) and compound 24B (61.1 mg, 99.6% purity, 32.0% yield, 99.9% stereopure) as white solids.

Compound 24A

LC-MS (ESI): R_{T}=4.104 min, mass calcd. for C₂₈H₂₆Cl₂F₃N₅O₄ 623.1, m/z found 624.1 [M+H]⁺. Chiral analysis (Column: Chiralpak IE 5 μm 4.6*250 mm; Mobile Phase: Hex:EtOH=40:60 at 1 mL/min; Temp: 30° C.; Wavelength: 254 nm, R_{T}=7.463 min). ¹H NMR (400 MHz, DMSO-d₆) δ 7.83 (d, J=4.4 Hz, 1H), 7.75 (d, J=8.4 Hz, 2H), 7.47-7.06 (m, 5H), 5.85-5.57 (m, 1H), 5.51-5.14 (m, 1H), 5.06-4.95 (m, 1H), 4.69-4.37 (m, 1H), 4.29-4.12 (m, 1H), 4.10-3.92 (m, 1H), 3.43-3.31 (m, 1H), 2.98-2.83 (m, 1H), 2.60-2.52 (m, 1H), 2.41 (d, J=4.4 Hz, 3H), 1.61-1.37 (m, 3H), 1.32-1.09 (m, 3H). ¹⁹F NMR 6 (376 MHz, DMSO-d₆) δ −82.60, −130.84.

Compound 24B

LC-MS (ESI): R_{T}=3.972 min, mass calcd. for C₂₈H₂₆Cl₂F₃N₅O₄ 623.1 m/z found 624.2 [M+H]⁺. Chiral analysis (Column: Chiralpak IE 5 μm 4.6*250 mm; Mobile Phase: Hex:EtOH=40:60 at 1 mL/min; Temp: 30° C.; Wavelength: 254 nm, R_{T}=9.853 min). ¹H NMR (400 MHz, DMSO-d₆) δ 8.02 (s, 1H), 7.74 (d, J=8.4 Hz, 2H), 7.46-7.05 (m, 5H), 5.85-5.56 (m, 1H), 5.51-5.15 (m, 1H), 5.09-4.95 (m, 1H), 4.67-4.41 (m, 1H), 4.29-4.07 (m, 1H), 3.77-3.54 (m, 2H), 2.98-2.83 (m, 1H), 2.63 (d, J=4.4 Hz, 3H), 2.59-2.52 (m, 1H), 1.51-1.34 (m, 3H), 1.30-1.09 (m, 3H). ¹⁹F NMR (376 MHz, DMSO-d₆) δ −82.32, −130.74.

Compound 25

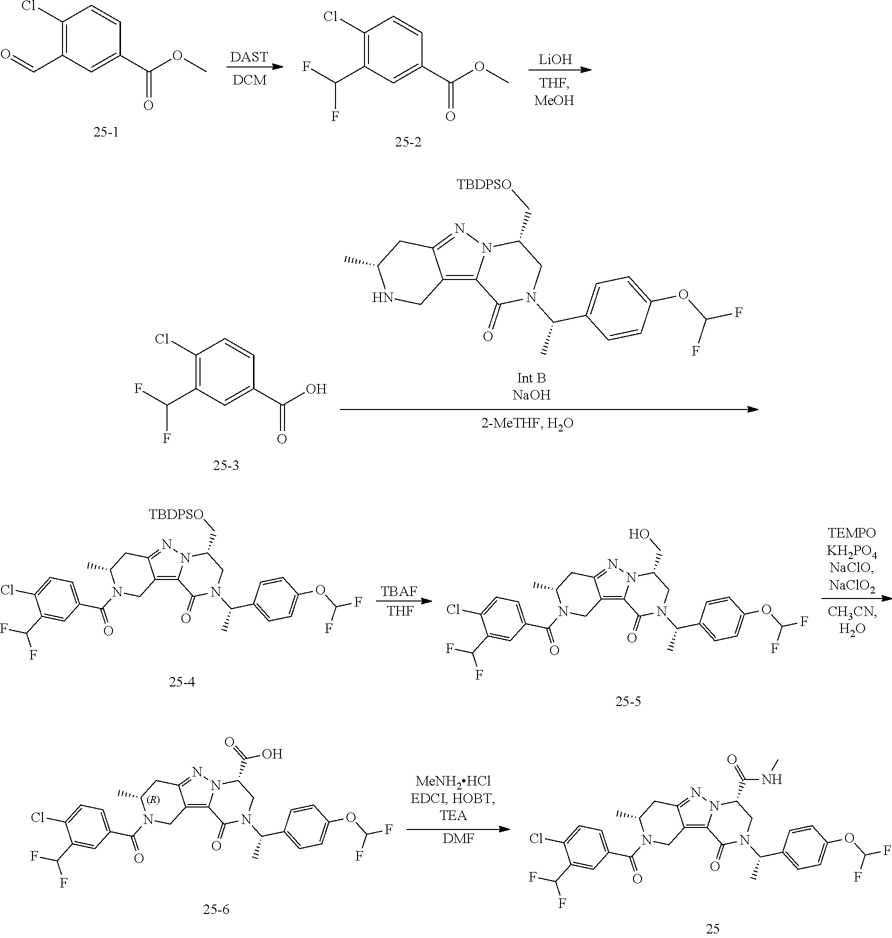

Intermediate 25-2

Methyl 4-chloro-3-(difluoromethyl)benzoate

To the solution of methyl 4-chloro-3-formylbenzoate 25-1 (200 mg, 1.01 mmol) in dichloromethane (2 mL) was added diethylaminosulfur trifluoride (0.5 mL, 3.79 mmol) at 0° C. After being stirred at room temperature overnight, the mixture was quenched with sodium bicarbonate aqueous solution (10 mL), and extracted with dichloromethane (10 mL) twice. The combined organic layers were washed with brine (20 mL), dried over Na₂SO₄₍ₛ₎ and filtered. The filtrate was concentrated and purified by silica gel column chromatography (petroleum ether:ethyl acetate=10:1) to give the title compound (120 mg, 95% purity from ¹H NMR, 51.3% yield) as yellow solids. ¹H NMR (400 MHz, CDCl₃) δ 8.34 (s, 1H), 8.09 (d, J=8.4 Hz, 1H), 7.52 (d, J=8.4 Hz, 1H), 6.96 (t, J=54.8 Hz, 1H), 3.95 (s, 3H).

Intermediate 25-3

4-Chloro-3-(difluoromethyl)benzoic acid

To a solution of methyl 4-chloro-3-(difluoromethyl)benzoate 25-2 (200 mg, 95% purity, 0.86 mmol) in tetrahydrofuran (2 mL) and methanol (2 mL) was added a solution of lithium hydroxide monohydrate (120 mg, 2.86 mmol) in water (2 mL) at 0° C. After being stirred 0° C. for 2 hours, the mixture was acidified to pH˜6 with 0.1 M hydrochloride aqueous solution and extracted with ethyl acetate (30 mL) twice. The combined organic layers were washed with water (30 mL) for three times and brine (30 mL), dried over Na₂SO₄₍ₛ₎ and filtered. The filtrate was concentrated under reduced pressure to give the title compound (180 mg, 95% purity from ¹H NMR, 96.1% yield) as yellow solids. ¹H NMR (400 MHz, CDCl₃) δ 13.47 (br s, 1H), 8.17 (s, 1H), 8.09 (d, J=8.8 Hz, 1H), 7.76 (d, J=8.8 Hz, 1H), 7.28 (t, J=54.4 Hz, 1H).

Intermediate 25-4

(3R,7S)-7-(((tert-Butyldiphenylsilyl)oxy)methyl)-2-(4-chloro-3-(difluoromethyl)benzoyl)-9-((S)-1-(4-(difluoromethoxy)phenyl)ethyl)-3-methyl-1,2,3,4,8,9-hexahydropyrido[4′,3′:3,4]pyrazolo[1,5-a]pyrazin-10(7H)-one

To the solution of (3R,7S)-7-(((tert-butyldiphenylsilyl)oxy)methyl)-9-((S)-1-(4-(difluoromethoxy)phenyl)ethyl)-3-methyl-1,2,3,4,8,9-hexahydropyrido[4′,3′:3,4]pyrazolo[1,5-a]pyrazin-10(7H)-one Int B (380 mg, 82.2% purity, 0.48 mmol) in N,N-dimethylformamide (5 mL) was added 4-chloro-3-(difluoromethyl)benzoic acid 25-3 (150 mg, 95% purity, 0.73 mmol), 2-(7-Aza-1H-benzotriazole-1-yl)-1,1,3,3-tetramethyluronium hexafluorophosphate aqueous solution (300 mg, 0.79 mmol) and trimethylamine (0.2 mL, 1.44 mmol) at 0° C. After being stirred at room temperature overnight, the mixture was diluted with water (30 mL), acidized with 0.5 M hydrochloric acid aqueous solution to pH˜6 and extracted with ethyl acetate (30 mL) twice. The combined organic layers were washed with brine (30 mL), dried over Na₂SO₄₍ₛ₎ and filtered. The filtrate was concentrated and purified by C18 column (acetonitrile:water=5% to 100%) to give the title compound (330 mg, 90% purity from ¹H NMR, 73.6% yield) as yellow solids. LC-MS (ESI): R_{T}=2.13 min, mass calcd. for C₄₄H₄₅ClF₄N₄O₄Si 832.3, m/z found 833.3 [M+H]⁺.

Intermediate 25-5

(3R,7S)-2-(4-Chloro-3-(difluoromethyl)benzoyl)-9-((S)-1-(4-(difluoromethoxy)phenyl)ethyl)-7-(hydroxymethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(4-chloro-3-(difluoromethyl)benzoyl)-9-((S)-1-(4-(difluoromethoxy)phenyl)ethyl)-3-methyl-1,2,3,4,8,9-hexahydropyrido[4′,3′:3,4]pyrazolo[1,5-a]pyrazin-10(7H)-one 25-4 (330 mg, 90% purity, 0.36 mmol) in tetrahydrofuran (3 mL) was added 1 M tetrabutylammonium fluoride in tetrahydrofuran (1.2 mL, 1.2 mmol) at 0° C. After being stirred at 0° C. for 1 hour, the mixture was concentrated under reduced pressure to give a crude. The crude was purified by C18 column (acetonitrile:water=30% to 70%) to give the title compound (200 mg, 90% purity from ¹H NMR, 84.9% yield) as yellow solids. LC-MS (ESI): R_{T}=1.59 min, mass calcd. for C₂₈H₂₇ClF₄N₄O₄ 594.2, m/z found 595.1 [M+H]⁺. ¹H NMR (400 MHz, CDCl₃) δ 7.75 (s, 1H), 7.50 (s, 2H), 7.34-7.33 (m, 2H), 7.13-6.82 (m, 3H), 6.52 (t, J=73.6 Hz, 1H), 6.11-5.94 (m, 1H), 5.82-5.31 (m, 1H), 5.03-4.26 (m, 3H), 4.04-3.92 (m, 2H), 3.55 (t, J=12.8 Hz, 1H), 3.20-2.96 (m, 3H), 2.72-2.59 (m, 1H), 1.74-1.57 (m, 3H), 1.28-1.24 (m, 3H).

Intermediate 25-6

(3R,7S)-2-(4-Chloro-3-(difluoromethyl)benzoyl)-9-((S)-1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-7-carboxylic acid

To a solution of (3R,7S)-2-(4-chloro-3-(difluoromethyl)benzoyl)-9-((S)-1-(4-(difluoromethoxy)phenyl)ethyl)-7-(hydroxymethyl)-3-methyl-1,2,3,4,8,9-hexahydropyrido[4′,3′:3,4]pyrazolo[1,5-a]pyrazin-10(7H)-one 25-5 (200 mg, 90% purity, 0.30 mmol) in acetonitrile (3 mL) was added 2,2,6,6-tetramethylpiperidinooxy (95 mg, 0.61 mmol), sodium chlorite (90 mg, 80% purity, 0.62 mmol), saturated potassium dihydrogenphosphate aqueous solution (3 mL) and sodium hypochlorite aqueous solution (0.70 mL, 5.5% purity, 0.65 mmol) at 0° C. After being stirred at 20° C. overnight, the mixture was diluted with sodium sulfite saturated solution (15 mL) and extracted with ethyl acetate (30 mL) twice. The combined organic layers were washed with brine (30 mL), dried over Na₂SO₄₍ₛ₎ and filtered. The filtrate was concentrated and purified by C18 (acetonitrile: water=30% to 55%) to give the desired product (165 mg, 90% purity from ¹H NMR, 80.6% yield) as white solids. LC-MS (ESI): R_{T}=1.219 min, mass calcd. for C₂₈H₂₅ClF₄N₄O₅ 608.1, m/z found 606.9 [M−H]⁻. ¹H NMR (400 MHz, CDCl₃) δ 7.73 (s, 1H), 7.53-7.48 (m, 2H), 7.37-7.26 (m, 2H), 7.12-6.83 (m, 3H), 6.50 (t, J=73.6 Hz, 1H), 6.13-6.01 (m, 1H), 5.93-5.43 (m, 1H). 4.97-4.31 (m, 3H), 3.82-3.75 (m, 1H), 3.50 (dd, J=14.0, 4.8 Hz, 1H), 2.89-2.67 (m, 2H), 1.50 (d, J=6.8 Hz, 3H), 1.28-1.24 (m, 3H).

Compound 25

(3R,7S)-2-(4-Chloro-3-(difluoromethyl)benzoyl)-9-((S)-1-(4-(difluoromethoxy)phenyl)ethyl)-N,3-dimethyl-10-oxo-1,2,3,4,7,8,9,10-octahydropyrido[4′,3′:3,4]pyrazolo[1,5-a]pyrazine-7-carboxamide (25)

To a solution of (3R,7S)-2-(4-chloro-3-(difluoromethyl)benzoyl)-9-((S)-1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-7-carboxylic acid 25-6 (165 mg, 90% purity, 0.24 mmol), methanamine hydrochloride (40 mg, 0.59 mmol), 1-ethyl-(3-(3-dimethylamino)propyl)-carbodiimide hydrochloride (100 mg, 0.52 mmol) and benzotriazol-1-ol (70 mg, 0.52 mmol) in N,N-dimethylformamide (3 mL) at 0° C. was added triethylamine (0.2 mL, 1.44 mmol). After being stirred at 30° C. under nitrogen atmosphere for 2 hours, the mixture was acidified to pH˜6 with 0.5 M hydrochloride aqueous solution and extracted with ethyl acetate (40 mL) twice. The combined organic layers were washed with water (30 mL) for three times and brine (30 mL), dried over Na₂SO₄₍ₛ₎ and filtered. The filtrate was concentrated under reduced pressure to give a residue, which was purified by C18 column (acetonitrile:water=55% to 75%) to give the title compound (82.5 mg, 98.6% purity, 53.6% yield) as white solids. LC-MS (ESI): R_{T}=4.413 min, mass calcd. for C₂₉H₂₈ClF₄N₅O₄ 621.2, m/z found 622.2 [M+H]⁺. ¹H NMR (400 MHz, CDCl₃) δ 7.73 (s, 1H), 7.53-7.48 (m, 2H), 7.40-7.34 (m, 2H), 7.11-7.10 (m, 2H), 6.97 (t, J=54.4 Hz, 1H), 6.50 (t, J=73.6 Hz, 1H), 5.99 (br s, 2H), 5.72-5.11 (m, 1H), 4.83-4.05 (m, 4H), 3.38 (dd, J=13.6, 4.8 Hz, 1H), 3.17-2.95 (m, 1H), 2.80 (d, J=4.8 Hz, 3H), 2.74-2.62 (m, 1H), 1.65-1.56 (m, 3H), 1.39-1.23 (m, 3H). ¹⁹F NMR (376 MHz, CDCl₃) δ −81.05.

Compounds 26A and 26B

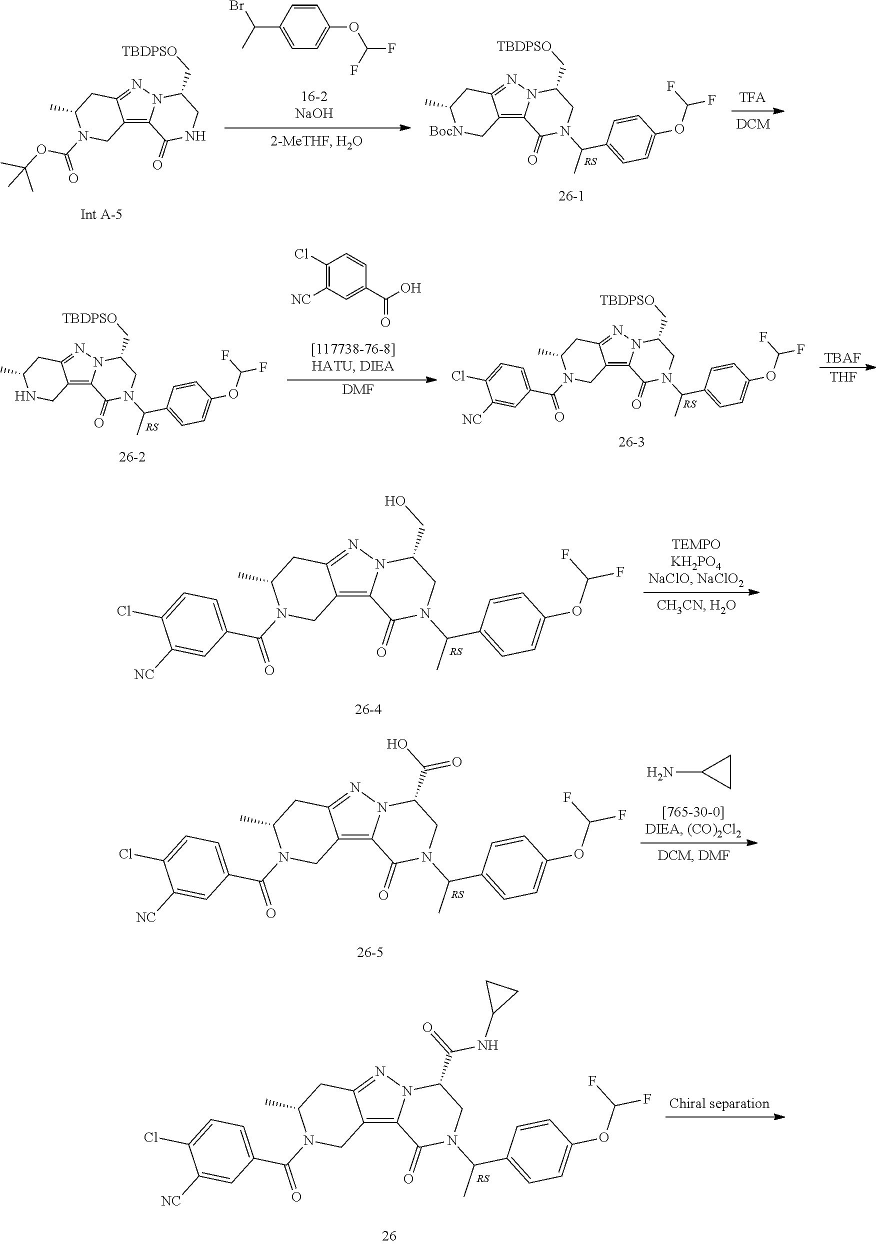

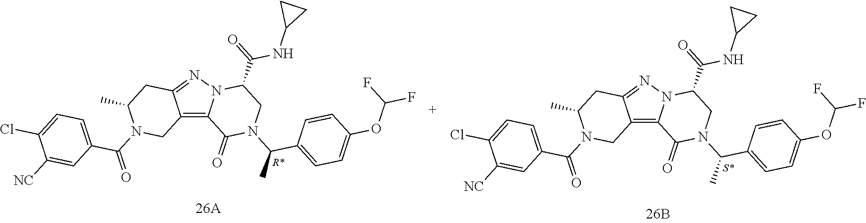 [SMILES: C[C@@H]1Cc2nn3c(c2CN1C(=O)c1ccc(Cl)c(C#N)c1)C(=O)N([C@H](C)c1ccc(OC(F)F)cc1)C[C@H]3C(=O)NC1CC1 ; C[C@@H]1Cc2nn3c(c2CN1C(=O)c1ccc(Cl)c(C#N)c1)C(=O)N([C@@H](C)c1ccc(OC(F)F)cc1)C[C@H]3C(=O)NC1CC1]

Intermediate 26-1

(3R,7S)-tert-Butyl 7-(((tert-butyldiphenylsilyl)oxy)methyl)-9-(1-(4-(difluoromethoxy)phenyl)ethyl)-3-methyl-10-oxo-3,4,7,8,9,10-hexahydropyrido[4′,3′:3,4]pyrazolo[1,5-a]pyrazine-2(1H)-carboxylate

To a solution of (3R,7S)-tert-butyl 7-(((tert-butyldiphenylsilyl)oxy)methyl)-3-methyl-10-oxo-3,4,7,8,9,10-hexahydropyrido[4′,3′:3,4]pyrazolo[1,5-a]pyrazine-2(1H)-carboxylate IntA-5 (1 g, 100% purity, 1.74 mmol) in 2-methyltetrahydrofuran (4 mL) was added 1-(1-bromoethyl)-4-(difluoromethoxy)benzene 16-2 (1.4 g, 5.02 mmol), benzyltriethylammonium chloride (198 mg, 0.869 mmol) and 50% sodium hydroxide in water (4 mL) at 0° C. After being stirred at 30° C. for 2 hours, the mixture was diluted with water (8 mL), extracted with ethyl acetate (10 mL) for three times. The combined organic layers were washed with brine (8 mL), dried over Na₂SO₄₍ₛ₎, filtered. The filtrate was concentrated and purified by C18 column (acetonitrile: water=5% to 100%) to give the title compound (940 mg, 100% purity from LCMS, 73% yield) as white solids. LC-MS (ESI): R_{T}=2.27 min and 2.33 min, mass calcd. for C₄₁H₅₀F₂N₄O₅Si 744.4, m/z found 745.0 [M+H]⁺.

Intermediate 26-2

(3R,7S)-7-(((tert-Butyldiphenylsilyl)oxy)methyl)-9-(1-(4-(difluoromethoxy)phenyl)ethyl)-3-methyl-1,2,3,4,8,9-hexahydropyrido[4′,3′:3,4]pyrazolo[1,5-a]pyrazin-10(7H)-one

To a solution of (3R,7S)-tert-butyl 7-(((tert-butyldiphenylsilyl)oxy)methyl)-9-(1-(4-(difluoromethoxy)phenyl)ethyl)-3-methyl-10-oxo-3,4,7,8,9,10-hexahydropyrido[4′,3′:3,4]pyrazolo[1,5-a]pyrazine-2(1H)-carboxylate 26-1 (940 mg, 100% purity, 1.26 mmol) in dichloromethane (4 mL) was added 2,2,2-trifluoroacetic acid (2 mL) at 0° C. After being stirred at 0° C. for 1 hour, the reaction mixture was quenched with saturated sodium bicarbonate (10 mL) to pH=10˜11, extracted with dichloromethane (15 mL) for three times. The organic layers were combined, washed with brine (10 mL), dried over Na₂SO₄₍ₛ₎ and filtered. The filtrate was concentrated to give the title compound (800 mg, 100% purity from LCMS, 98% yield) as colorless oil. LC-MS (ESI): R_{T}=0.77 and 0.82 min, mass calcd. for C₃₆H₄₂F₂N₄O₃Si 644.3, m/z found 645.5 [M+H]⁺.

Intermediate 26-3

5-((3R,7S)-7-(((tert-Butyldiphenylsilyl)oxy)methyl)-9-(1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-2-carbonyl)-2-chlorobenzonitrile

To a solution of (3R,7S)-7-(((tert-butyldiphenylsilyl)oxy)methyl)-9-(1-(4-(difluoromethoxy)phenyl)ethyl)-3-methyl-1,2,3,4,8,9-hexahydropyrido[4′,3′:3,4]pyrazolo[1,5-a]pyrazin-10(7H)-one 26-2 (800 mg, 100% purity, 1.24 mmol) in N,N-dimethylformamide (12 mL) was added 4-chloro-3-cyanobenzoic acid (293 mg, 1.61 mmol), 2-(3H-[1,2,3]triazolo[4,5-b]pyridin-3-yl)-1,1,3,3-tetramethylisouronium hexafluorophosphate(V) (613 mg, 1.61 mmol), N-ethyl-N-isopropylpropan-2-amine (0.8 mL, 4.95 mmol) at 0° C. After being stirred at room temperature overnight, the mixture was added water (50 mL), extracted with ethyl acetate (25 mL) for three times. The organic layers were washed with brine (25 mL), dried over Na₂SO₄₍ₛ₎, filtered and concentrated to give the title compound (900 mg, 99% purity from LCMS, 88% yield) as pale yellow solids. LC-MS (ESI): R_{T}=2.12 min and 2.17 min, mass calcd. for C₄₄H₄₄ClF₂N₅O₄Si 807.3, m/z found 808.5 [M+H]⁺.

Compound 26-4

2-Chloro-5-((3R,7S)-9-(1-(4-(difluoromethoxy)phenyl)ethyl)-7-(hydroxymethyl)-3-methyl-10-oxo-1,2,3,4,7,8,9,10-octahydropyrido[4′,3′:3,4]pyrazolo[1,5-a]pyrazine-2-carbonyl)benzonitrile

To a solution of 5-((3R,7S)-7-(((tert-butyldiphenylsilyl)oxy)methyl)-9-(1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-2-carbonyl)-2-chlorobenzonitrile 26-3 (900 mg, 99% purity, 1.10 mmol) in tetrahydrofuran (2 mL) was added 1 M tetrabutylammonium fluoride in tetrahydrofuran (1.5 mL, 1.5 mmol) at 0° C. After being stirred at room temperature for 2 hours, the mixture was diluted with water (5 mL) and extracted with ethyl acetate (10 mL) for three times. The combined organic layers were dried over Na₂SO₄₍ₛ₎ and filtered. The filtrate was concentrated and purified by C18 column (acetonitrile:water=5% to 100%) to give the title compound (600 mg, 98% purity from LCMS, 94% yield) as pale yellow solids. LC-MS (ESI): R_{T}=1.60 min, mass calcd. for C₂₈H₂₆ClF₂N₅O₄ 569.2, m/z found 570.2 [M+H]⁺.

Intermediate 26-5

(3R,7S)-2-(4-chloro-3-cyanobenzoyl)-9-(1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-7-carboxylic acid

To a solution of 2-chloro-5-((3R,7S)-9-(1-(4-(difluoromethoxy)phenyl)ethyl)-7-(hydroxymethyl)-3-methyl-10-oxo-1,2,3,4,7,8,9,10-octahydropyrido[4′,3′:3,4]pyrazolo[1,5-a]pyrazine-2-carbonyl)benzonitrile 26-4 (3 g, 90% purity, 4.74 mmol) in acetonitrile (25 mL) was added saturated potassium dihydrogen phosphate aqueous solution (25 mL), 2,2,6,6-tetramethylpiperidinooxy (1.5 g, 9.60 mmol), sodium chlorite (1.1 g, 9.73 mmol), dropwise 5.5% sodium hypochlorite aqueous solution (11.3 mL, 10.4 mmol) at 0° C. The reaction was allowed to slowly return to room temperature. After being stirred at room temperature overnight, the reaction mixture was quenched with saturated sodium thiosulfate (15 mL), acidized with 1 M hydrochloric acid solution to pH=4-5, extracted with ethyl acetate (30 mL) for three times. The combined organic layers were washed with brine (30 mL), dried over Na₂SO₄₍ₛ₎, filtered. The filtrate was concentrated and purified by C18 column (acetonitrile:water=5% to 35%) to give the title compound (2.0 g, 100% purity, 72.3% yield) as yellow solids. LC-MS (ESI): R_{T}=1.31 min, mass calcd. for C₂₈H₂₄ClF₂N₅O₅ 583.1, m/z found 583.9 [M+H]⁺.

Compound 26

(3R,7S)-2-(4-Chloro-3-cyanobenzoyl)-N-cyclopropyl-9-(1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-7-carboxamide

To a solution of (3R,7S)-2-(4-chloro-3-cyanobenzoyl)-9-(1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-7-carboxylic acid 26-5 (100 mg, 100% purity, 0.171 mmol) in dichloromethane (2 mL) was added oxalyl chloride (59 mg, 0.465 mmol and N,N-dimethylformamide (5 mg, 0.068 mmol) at 0° C. under nitrogen atmosphere. After being stirred at 0° C. for 1 hour, the reaction mixture was concentrated to give crude peak1. To a mixture of cyclopropanamine (53 mg, 0.928 mmol) and N-ethyl-N-isopropylpropan-2-amine (60 mg, 0.464 mmol) in dichloromethane (2 mL) was added peak1. After being stirred at 0° C. for 1 hour, the mixture was diluted with water (10 mL) and extracted with ethyl acetate (10 mL) twice. The combined organic layers were washed with water (10 mL) for three times and brine (10 mL), dried over Na₂SO₄₍ₛ₎ and filtered. The filtrate was concentrated under reduced pressure to give the residue, which was purified by C18 (acetonitrile:water=40% to 65%) to give the desired product (65 mg, 100% purity, 60.9% yield) as white solids. LC-MS (ESI): R_{T}=1.59 min, mass calcd. for C₃₁H₂₉ClF₂N₆O₄ 622.2, m/z found 622.9 [M+H]⁺.

Compounds 26A and 26B

(3R,7S)-2-(4-Chloro-3-cyanobenzoyl)-N-cyclopropyl-9-((R*)-1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-7-carboxamide (26A), and (3R,7S)-2-(4-chloro-3-cyanobenzoyl)-N-cyclopropyl-9-((S*)-1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-7-carboxamide (26B)

A racemic mixture of (3R,7S)-2-(4-chloro-3-cyanobenzoyl)-N-cyclopropyl-9-(1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-7-carboxamide 26 (65 mg, 100% purity, 0.104 mmol) was separated by chiral Prep. HPLC separation condition: (Column: Chiralpak IE 5 μm 20*250 mm; Mobile Phase: Hex:EtOH=40:60 at 30 mL/min; Temp: 30° C.; Wavelength: 254 nm) to give the title compounds 26A (6.4 mg, 99.6% purity, 9.8% yield, 100% stereopure) as white solids and 26B (13.1 mg, 99.5% purity, 20.1% yield, 100% stereopure) as white solids.

Compound 26A

LC-MS (ESI): R_{T}=9.516 min, mass calcd. for C₃₁H₂₉ClF₂N₆O₄ 622.2, m/z found 623.2 [M+H]⁺. Chiral analysis (Column: Chiralpak IE 5 μm 4.6*250 mm; Mobile Phase: Hex:EtOH=40:60 at 1 mL/min; Temp: 30° C.; Wavelength: 254 nm; R_{T}=9.561 min). ¹H NMR (400 MHz, CDCl₃) δ 7.74 (s, 1H), 7.60 (s, 2H), 7.31 (d, J=8.4 Hz, 2H), 7.10 (d, J=8.4 Hz, 2H), 6.51 (t, J=73.6 Hz, 1H), 6.18-5.91 (m, 2H), 5.47 (br s, 1H), 4.82-4.52 (m, 3H), 3.86 (s, 2H), 3.10 (br s, 1H), 2.74-2.70 (m, 1H), 2.54 (s, 1H), 1.59-1.58 (m, 3H), 1.30-1.29 (m, 3H), 0.74-0.72 (m, 2H), 0.36-0.26 (m, 2H). ¹⁹F NMR (376 MHz, CDCl₃) δ −80.87.

Compound 26B

LC-MS (ESI): R_{T}=9.737 min, mass calcd. for C₃₁H₂₉ClF₂N₆O₄ 622.2, m/z found 623.2 [M+H]⁺. Chiral analysis (Column: Chiralpak IE 5 μm 4.6*250 mm; Mobile Phase: Hex:EtOH=40:60 at 1 mL/min; Temp: 30° C.; Wavelength: 254 nm; R_{T}=13.489 min). ¹H NMR (400 MHz, CDCl₃) δ 7.74 (s, 1H), 7.61 (s, 2H), 7.36-7.34 (m, 2H), 7.10 (d, J=8.4 Hz, 2H), 6.50 (t, J=73.6 Hz, 1H), 6.13-6.00 (m, 2H), 5.50 (br s, 1H), 4.79-4.54 (m, 3H), 4.08-4.05 (m, 1H), 3.42-3.56 (m, 1H), 3.07 (br s, 1H), 2.74-2.65 (m, 2H), 1.59-1.58 (m, 3H), 1.30-1.29 (m, 3H), 0.81-0.76 (m, 2H), 0.41-0.37 (m, 2H). ¹⁹F NMR (376 MHz, CDCl₃) δ −81.62.

Compounds 27A and 27B (Preparation Method A)

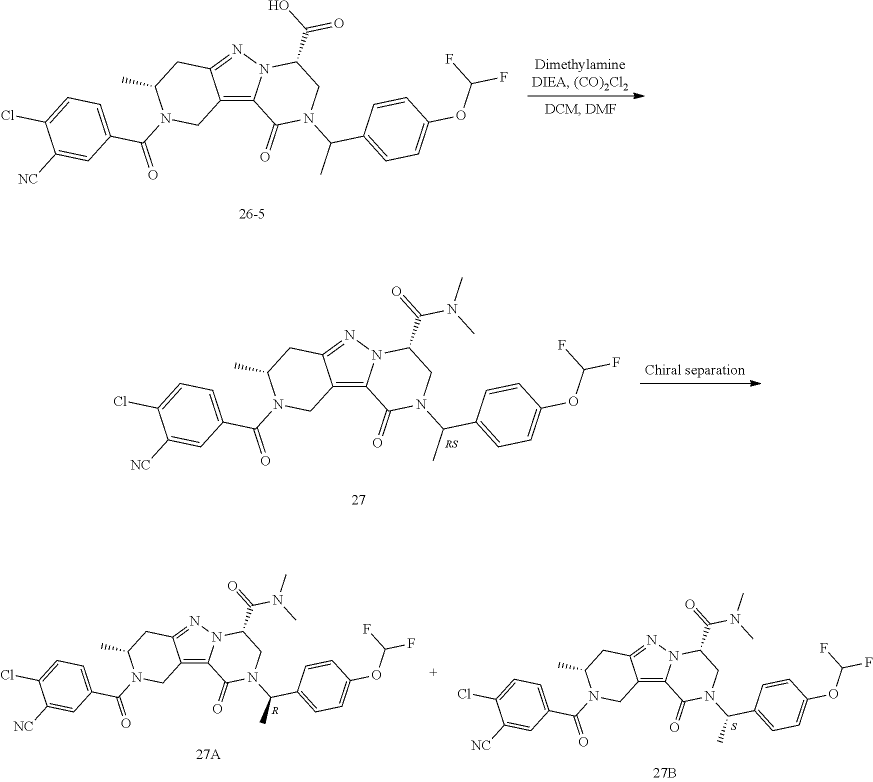

Compound 27

(3R,7S)-2-(4-Chloro-3-cyanobenzoyl)-9-(1-(4-(difluoromethoxy)phenyl)ethyl)-N,N,3-trimethyl-10-oxo-1,2,3,4,7,8,9,10-octahydropyrido[4′,3′:3,4]pyrazolo[1,5-a]pyrazine-7-carboxamide

To a solution of (3R,7S)-2-(4-chloro-3-cyanobenzoyl)-9-(1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-7-carboxylic acid 26-5 (100 mg, 100% purity, 0.171 mmol) in dichloromethane (2 mL) was added oxalyl chloride (59 mg, 0.465 mmol) and N,N-dimethylformamide (5 mg, 0.068 mmol) at 0° C. under nitrogen atmosphere. After being stirred at 0° C. for 1 hour, the reaction mixture was concentrated to give crude peak1. To a mixture of 1 M dimethylamine in tetrahydrofuran (1 mL, 1 mmol) and N-ethyl-N-isopropylpropan-2-amine (60 mg, 0.464 mmol) in dichloromethane (2 mL) was added peak1. After being stirred at 0° C. for 1 hour, the mixture was diluted with water 10 mL and extracted with ethyl acetate (10 mL) twice. The combined organic layers were washed with water (10 mL) for three times and brine (10 mL), dried over Na₂SO₄₍ₛ₎ and filtered. The filtrate was concentrated under reduced pressure to give the residue, which was purified by C18 (acetonitrile:water=40% to 65%) to give the desired product (70 mg, 100% purity, 66.9% yield) as white solids. LC-MS (ESI): R_{T}=1.57 min, mass calcd. for C₃₀H₂₉ClF₂N₆O₄ 610.2, m/z found 610.8 [M+H]⁻.

Compounds 27A and 27B

(3R,7S)-2-(4-Chloro-3-cyanobenzoyl)-9-((R)-1-(4-(difluoromethoxy)phenyl)ethyl)-N,N,3-trimethyl-10-oxo-1,2,3,4,7,8,9,10-octahydropyrido[4′,3′:3,4]pyrazolo[1,5-a]pyrazine-7-carboxamide (27A), and (3R,7S)-2-(4-chloro-3-cyanobenzoyl)-9-((S)-1-(4-(difluoromethoxy)phenyl)ethyl)-N,N,3-trimethyl-10-oxo-1,2,3,4,7,8,9,10-octahydropyrido[4′,3′:3,4]pyrazolo[1,5-a]pyrazine-7-carboxamide (27B)

A racemic mixture of (3R,7S)-2-(4-chloro-3-cyanobenzoyl)-9-(1-(4-(difluoromethoxy)phenyl)ethyl)-N,N,3-trimethyl-10-oxo-1,2,3,4,7,8,9,10-octahydropyrido[4′,3′:3,4]pyrazolo[1,5-a]pyrazine-7-carboxamide 27 (70 mg, 100% purity, 0.115 mmol) was separated by chiral Prep. HPLC separation condition: (Column: Chiralpak IA 5 μm 20*250 mm; Mobile Phase: CO₂: IPA (0.2% DEA)=60:40 at 30 g/min; Col. Temp: 40° C.; Wavelength: 254 nm, Back pressure: 100 bar) to give the title compounds 27A (13.8 mg, 99.7% purity, 19.7% yield, 99.4% stereopure) as white solids and 27B (22.9 mg, 99.8% purity, 32.6% yield, 100% stereopure) as white solids.

Compound 27A

LC-MS (ESI): R_{T}=9.422 min, mass calcd. for C₃₀H₂₉ClF₂N₆O₄ 610.2, m/z found 611.2 [M+H]⁺. Chiral analysis (Column: Chiralpak IA 5 μm 4.6*250 mm; Mobile Phase: CO₂: IPA (0.2% DEA)=60:40 at 3 g/min; Temp: 40° C.; Wavelength: 254 nm; Back Pressure: 100 Bar; R_{T}=6.4 min). ¹H NMR (400 MHz, CDCl₃) δ 7.74 (s, 1H), 7.63-7.58 (s, 2H), 7.31 (d, J=8.0 Hz, 2H), 7.12 (d, J=8.4 Hz, 2H), 6.53 (t, J=73.2 Hz, 1H), 5.97 (br s, 1H), 5.77-5.4 (m, 1H), 5.25-5.23 (m, 1H), 4.67-4.63 (m, 2H), 3.91-3.87 (m, 1H), 3.34-3.30 (m, 1H), 3.07 (br s, 1H), 2.73-2.69 (m, 7H), 1.56-1.53 (m, 3H), 1.33-1.32 (m, 3H). ¹⁹F NMR (376 MHz, CDCl₃) δ −81.29.

Compound 27B

LC-MS (ESI): R_{T}=9.578 min, mass calcd. for C₃₀H₂₉ClF₂N₆O₄ 610.2, m/z found 611.2 [M+H]⁺. Chiral analysis (Column: Chiralpak IA 5 μm 4.6*250 mm; Mobile Phase: CO₂: IPA (0.2% DEA)=60:40 at 3 g/min; Temp: 40° C.; Wavelength: 254 nm; Back Pressure: 100 Bar; R_{T}=4.19 min). ¹H NMR (400 MHz, CDCl₃) δ 7.73 (s, 1H), 7.63-7.58 (s, 2H), 7.32 (m, 2H), 7.11 (d, J=8.4 Hz, 2H), 6.52 (t, J=73.6 Hz, 1H), 6.02 (br s, 1H), 5.78-5.38 (m, 1H), 5.12 (s, 1H), 4.80-4.56 (m, 2H), 3.79-3.74 (m, 1H), 3.37-3.32 (m, 1H), 3.11-3.03 (m, 7H), 2.72-2.68 (m, 1H), 1.53-1.51 (m, 3H), 1.30-1.28 (m, 3H). ¹⁹F NMR (376 MHz, CDCl₃) δ −81.22.

Compound 27B (Preparation Method B)

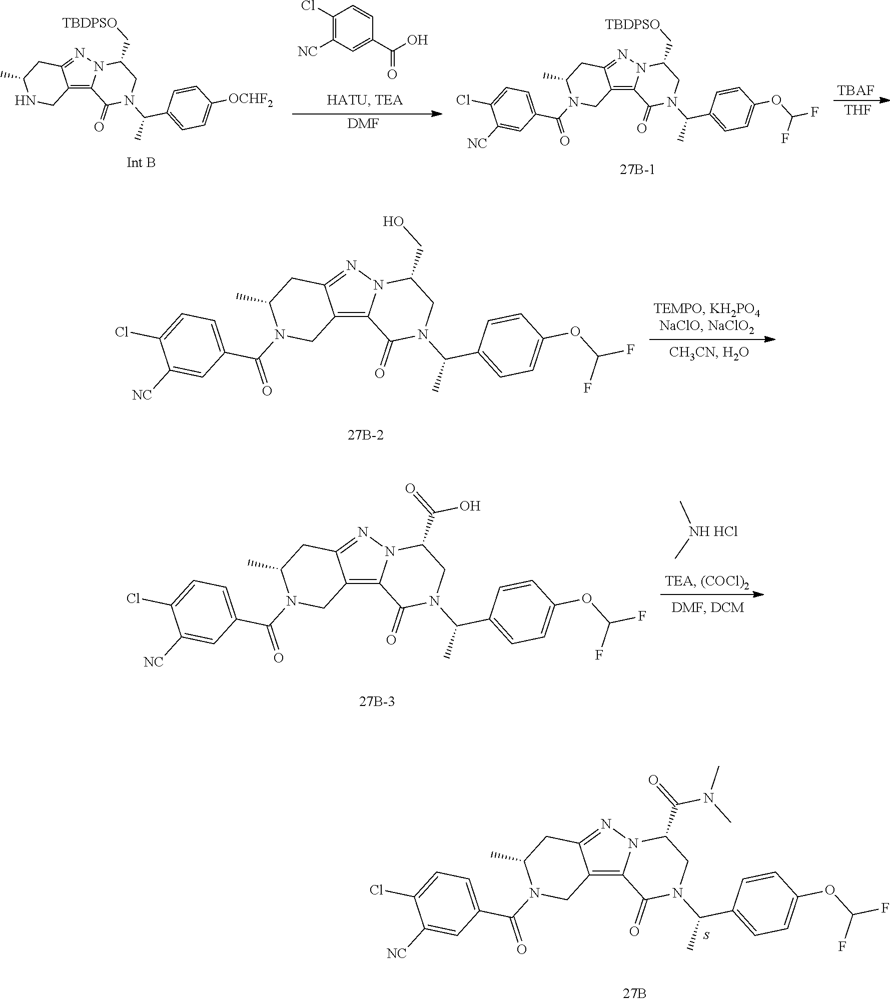

Intermediate 27B-1

5-((3R,7S)-7-(((tert-Butyldiphenylsilyl)oxy)methyl)-9-((S)-1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-2-carbonyl)-2 chlorobenzonitrile

To the solution of (3R,7S)-7-(((tert-butyldiphenylsilyl)oxy)methyl)-9-((S)-1-(4-(difluoromethoxy)phenyl)ethyl)-3-methyl-1,2,3,4,8,9-hexahydropyrido[4′,3′:3,4]pyrazolo[1,5-a]pyrazin-10(7H)-one Int B (9.7 g, 100% purity, 14.2 mmol) in N,N-dimethylformamide (100 mL) were added 4-chloro-3-cyanobenzoic acid (3 g, 16.5 mmol), 2-(7-aza-1H-benzotriazole-1-yl)-1,1,3,3-tetramethyluronium hexafluorophosphate (8.2 g, 21.6 mmol) and triethylamine (6 mL, 43.0 mmol) at 0° C. After stirred at room temperature for 20 minutes, the mixture was diluted with water (500 mL), acidized with 0.5 M hydrochloric acid aqueous solution to pH˜6 and extracted with ethyl acetate (500 mL) twice. The combined organic layers were washed with brine (500 mL), dried over Na₂SO₄₍ₛ₎ and filtered. The filtrate was concentrated and purified by C18 column (acetonitrile:water=5% to 100%) to give the title compound (8 g, 100% purity from LCMS, 69.5% yield) as yellow solids. LC-MS (ESI): R_{T}=2.02 min, mass calcd. for C₄₄H₄₄ClF₂N₅O₄Si 807.3, m/z found 807.8 [M+H]⁺.

Intermediate 27B-2

2-Chloro-5-((3R,7S)-9-((S)-1-(4-(difluoromethoxy)phenyl)ethyl)-7-(hydroxymethyl)-3-methyl-10-oxo-1,2,3,4,7,8,9,10-octahydropyrido[4′,3′:3,4]pyrazolo[1,5-a]pyrazine-2-carbonyl)benzonitrile

To a solution of 5-((3R,7S)-7-(((tert-butyldiphenylsilyl)oxy)methyl)-9-((S)-1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-2-carbonyl)-2-chlorobenzonitrile 27B-1 (8 g, 100% purity, 9.90 mmol) in tetrahydrofuran (80 mL) was added 1 M tetrabutylammonium fluoride in tetrahydrofuran (14.5 mL, 14.5 mmol) at 0° C. After stirred at 0° C. for 1 hour, the mixture was concentrated under reduced pressure to give a residue, which was purified by C18 column (acetonitrile:water=5% to 100%) to give the crude compound. The crude compound was added acetonitrile (20 mL) and stirred at room temperature for 20 minutes and filtered. The obtained white solids were collected by filtration to give the title compound (5.5 g, 100% purity from LCMS, 97.5% yield, 100% stereopure) as white solids. LC-MS (ESI): R_{T}=1.57 min, mass calcd. for C₂₈H₂₆ClF₂N₅O₄ 569.2, m/z found 569.8 [M+H]⁺. Chiral analysis (Column: Chiralpak IA 5 μm 4.6*250 m Back pressure: 100 bar; Mobile Phase: CO₂: EtOH=60:40 at 3 g/min; Col. Temp: 40° C.; Wavelength: 214 nm, R_{T}=3.6 min).

Intermediate 27B-3

(3R,7S)-2-(4-Chloro-3-cyanobenzoyl)-9-((S)-1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-7-carboxylic acid

To a solution of 2-chloro-5-((3R,7S)-9-((S)-1-(4-(difluoromethoxy)phenyl)ethyl)-7-(hydroxymethyl)-3-methyl-10-oxo-1,2,3,4,7,8,9,10-octahydropyrido[4′,3′:3,4]pyrazolo[1,5-a]pyrazine-2-carbonyl)benzonitrile 27B-2 (5.5 g, 100% purity, 9.65 mmol) in acetonitrile (100 mL) were added 2,2,6,6-tetramethylpiperidinooxy (3 g, 19.2 mmol), sodium chlorite (2.2 g, 80% purity, 19.5 mmol), saturated potassium dihydrogenphosphate aqueous solution (100 mL) and sodium hypochlorite (12 mL, 20.2 mmol) at 0° C. After stirred at 0° C. overnight, the mixture was diluted with sodium sulfite saturated solution (50 mL) and extracted with ethyl acetate (150 mL) twice. The combined organic layers were washed with brine (100 mL), dried over Na₂SO₄₍ₛ₎ and filtered. The filtrate was concentratede to give a residue, which was added acetonitrile (20 mL) and stirred at room temperature for 20 minutes and filtered. The obtained white solids were collected by filtration to give the title compound (5.6 g, 100% purity from LCMS, 99.4% yield, 100% stereopure). LC-MS (ESI): R_{T}=1.25 min mass calcd. for C₂₈H₂₄ClF₂N₅O₅ 583.1, m/z found 584.1 [M+H]⁺. Chiral analysis (Column: Chiralpak IA 5 μm 4.6*250 m Back pressure: 100 bar; Mobile Phase: CO₂: EtOH (0.2% TFA)=60:40 at 3 g/min; Col. Temp: 40° C.; Wavelength: 254 nm, R_{T}=2.65 min).

Compound 27B

(3R,7S)-2-(4-Chloro-3-cyanobenzoyl)-9-((S)-1-(4-(difluoromethoxy)phenyl)ethyl)-N,N,3-trimethyl-10-oxo-1,2,3,4,7,8,9,10-octahydropyrido[4′,3′:3,4]pyrazolo[1,5-a]pyrazine-7-carboxamide

To a solution of (3R,7S)-2-(4-chloro-3-cyanobenzoyl)-9-((S)-1-(4-(difluoromethoxy) phenyl)ethyl)-3-methyl-10-oxo-1,2,3,4,7,8,9,10-octahydropyrido[4′,3′:3,4]pyrazolo [1,5-a]pyrazine-7-carboxylic acid 27B-3 (200 mg, 100% purity, 0.342 mmol) in dichloromethane (4 mL) were added oxalyl chloride (66 mg, 0.520 mmol) and N,N-dimethylformamide (5 mg, 0.068 mmol) at 0° C. under nitrogen atmosphere. After stirred at room temperature for 1 hour, the mixture was concentrated to give yellow solids A. To the solution of 2 M dimethylamine in tetrahydrofuran (0.4 mL, 0.8 mmol) in dichloromethane (4 mL) were added the solution of A in dichloromethane (4 mL) and triethylamine (120 mg, 1.19 mmol) at 0° C. dropwise. After stirred at room temperature for 2 hours, the solution was diluted with dichloromethane (20 mL), washed with water (10 mL) and brine (10 mL), dried over Na₂SO₄₍ₛ₎, filtered. The filtrate was concentrated, purified by C18 column (acetonitrile:water=50% to 70%) to give the title compound (200 mg, 100% purity from LCMS, 96% yield, 97.3% stereopure) as white solids. LC-MS (ESI): R_{T}=1.58 min, mass calcd. for C₃₀H₂₉ClF₂N₆O₄ 610.2, m/z found 611.2 [M+H]⁺. Chiral analysis (Column: Chiralpak OJ-H 5 μm 4.6*250 mm; Mobile Phase: Hex:EtOH=40:60 at 1 mL/min; Temp: 30° C.; Wavelength: 254 nm; Rt=8.574 min).

Crude 27B (300 mg, 97% purity, 0.476 mmol, from 2 batches) was separated by chiral Prep. HPLC (separation condition: Column: Chiralpak OJ-H 5 μm 30*250 mm; Mobile Phase: hexane:EtOH=40:60 at 25 mL/min; Col. Temp: 30° C.; Wavelength: 254 nm) to afford the crude, which was purified by C18 column (acetonitrile:water (+0.2% ammonium bicarbonate)=50% to 70%) to give the title compound 27B in two fractions: Fraction 1 (100 mg, 99.7% purity from LCMS, 34% yield, 99.8% stereopure) as white solids and Fraction 2 (130 mg, 99.2% purity from LCMS, 44% yield, 99.8% stereopure) as white solids.

Fraction 1: LC-MS (ESI): R_{T}=8.532 min, mass calcd. for C₃₀H₂₉ClF₂N₆O₄ 610.2, m/z found 611.2 [M+H]⁺. Chiral analysis (Column: Chiralpak OJ-H 5 μm 4.6*250 mm; Mobile Phase: Hex:EtOH=40:60 at 1 mL/min; Temp: 30° C.; Wavelength: 254 nm; Rt=8.672 min). ¹H NMR (400 MHz, CDCl₃) δ 7.73 (s, 1H), 7.62-7.59 (m, 2H), 7.32 (d, J=7.2 Hz, 2H), 7.12 (d, J=7.2 Hz, 2H), 6.52 (t, J=73.6 Hz, 1H), 5.99 (br s, 1H), 5.82-5.33 (m, 1H), 5.11 (s, 1H), 4.85-4.18 (m, 2H), 3.76 (q, J=6.8 Hz, 1H), 3.35 (d, J=11.2 Hz, 1H), 3.11-3.03 (m, 7H), 2.70 (d, J=15.6 Hz, 1H), 1.52 (d, J=7.2 Hz, 3H), 1.29 (d, J=5.6 Hz, 3H). ¹⁹F NMR (376 MHz, CDCl₃) δ −81.21.

Fraction 2: LC-MS (ESI): R_{T}=8.543 min, mass calcd. for C₃₀H₂₉ClF₂N₆O₄ 610.2, m/z found 611.2 [M+H]⁺. Chiral analysis (Column: Chiralpak OJ-H 5 μm 4.6*250 mm; Mobile Phase: Hex:EtOH=40:60 at 1 mL/min; Temp: 30° C.; Wavelength: 254 nm; Rt=8.697 min). ¹H NMR (400 MHz, CDCl₃) δ 7.73 (s, 1H), 7.63-7.58 (m, 2H), 7.32 (d, J=7.2 Hz, 2H), 7.12 (d, J=8.4 Hz, 2H), 6.52 (t, J=73.2 Hz, 1H), 5.99 (br s, 1H), 5.79-5.38 (m, 1H), 5.11 (s, 1H), 4.79-4.25 (m, 2H), 3.76 (q, J=6.8 Hz, 1H), 3.35 (d, J=10.8 Hz, 1H), 3.11-3.03 (m, 7H), 2.70 (d, J=16.4 Hz, 1H), 1.52 (d, J=7.2 Hz, 3H), 1.29 (d, J=5.6 Hz, 3H)¹⁹F NMR (376 MHz, CDCl₃) δ −81.21.

Compound 28

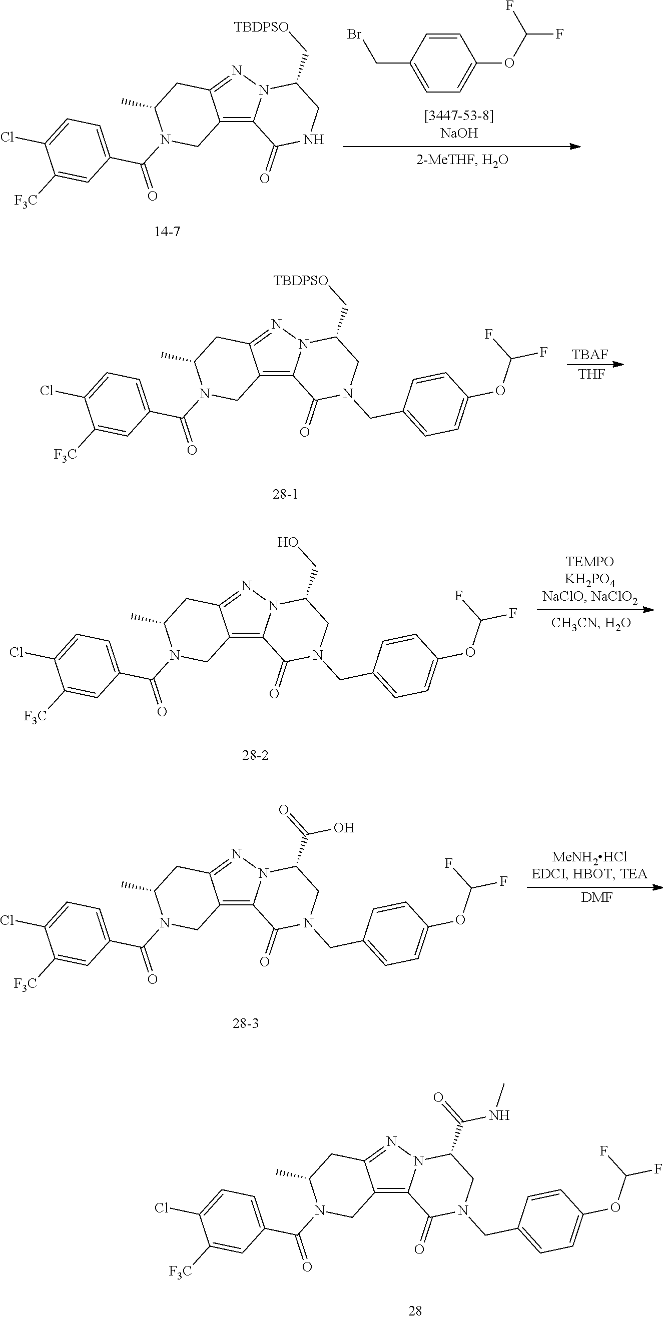

Intermediate 28-1

(3R,7S)-7-(((tert-Butyldiphenylsilyl)oxy)methyl)-2-(4-chloro-3-(trifluoromethyl)benzoyl)-9-(4-(difluoromethoxy)benzyl)-3-methyl-1,2,3,4,8,9-hexahydropyrido[4′,3′:3,4] pyrazolo[1,5-a]pyrazin-10(7H)-one

To a solution of (3R,7S)-7-(((tert-butyldiphenylsilyl)oxy)methyl)-2-(3-chloro-4-(trifluoromethyl)benzoyl)-3-methyl-1,2,3,4,8,9-hexahydropyrido[4′,3′:3,4]pyrazolo[1,5-a]pyrazin-10(7H)-one 14-7 (200 mg, 0.294 mmol, 100% purity) and 1-(bromomethyl)-4-(difluoromethoxy)benzene (130 mg, 0.548 mmol) in 2-methyltetrahydrofuran (5 mL) was added 50% wt. sodium hydroxide in water (5 mL) slowly at 30° C. After being stirred at 30° C. for 2 hours, the mixture was diluted with water (50 mL) and concentrated at room temperature under reduced pressure to remove the volatile. The remained aqueous layer was acidified with 2 M hydrochloride aqueous solution (20 mL) and extracted with ethyl acetate (30 mL) twice and brine (60 mL), dried over Na₂SO₄₍ₛ₎ and filtered. The filtrate was concentrated to give the crude (250 mg, 83% purity, 84% yield) as yellow oil. LC-MS (ESI): R_{T}=2.27 min, mass calcd. for C₄₃H₄₂ClF₅N₄O₄Si 836.3, m/z found 837.2 [M+H]⁺.

Intermediate 28-2

(3R,7S)-2-(4-Chloro-3-(trifluoromethyl)benzoyl)-9-(4-(difluoromethoxy)benzyl)-7-(hydroxymethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3-chloro-4-(trifluoromethyl)benzoyl)-9-(4-(difluoromethoxy)benzyl)-3-methyl-1,2,3,4,8,9-hexahydropyrido[4′,3′:3,4]pyrazolo[1,5-a]pyrazin-10(7H)-one 28-1 (370 mg, 83% purity, 0.367 mmol) in tetrahydrofuran (3 mL) was added 1 M tetrabutylammonium fluoride (0.5 mL, 0.5 mmol) in tetrahydrofuran at 0° C. After being stirred at 0° C. for 4 hours, the mixture was filtered and concentrated under reduced pressure to give the crude. The crude was purified by C18 column (acetonitrile:water=5% to 100%) to give the title compound (180 mg, 95% purity, 78% yield) as yellow oil. LC-MS (ESI): R_{T}=1.63 min, mass calcd. for C₂₇H₂₄ClF₅N₄O₄ 598.1, m/z found 599.1 [M+H]⁺.

Intermediate 28-3

(3R,7S)-2-(4-Chloro-3-(trifluoromethyl)benzoyl)-9-(4-(difluoromethoxy)benzyl)-3-methyl-10-oxo-1,2,3,4,7,8,9,10-octahydropyrido[4′,3′:3,4]pyrazolo[1,5-a]pyrazine-7-carboxylic acid

To a solution of (3R,7S)-2-(3-chloro-4-(trifluoromethyl)benzoyl)-9-(4-(difluoromethoxy)benzyl)-7-(hydroxymethyl)-3-methyl-1,2,3,4,8,9-hexahydropyrido[4′,3′:3,4]pyrazolo[1,5-a]pyrazin-10(7H)-one 28-2 (180 mg, 95% purity, 0.286 mmol) in acetonitrile (4 mL) was added 2,2,6,6-tetramethylpiperidinooxy (90 mg, 0.576 mmol), sodium hypochlorite aqueous solution (0.35 mL, 0.588 mmol), Sodium chlorite (65 mg, 0.575 mmol), saturated sodium dihydrogenphosphate aqueous solution (4 mL) at 0° C. After being stirred at 0° C. for 1 hour, the mixture was diluted with water (10 mL) and extracted with ethyl acetate (20 mL) twice. The combined organic layers were washed with brine (30 mL), dried over Na₂SO₄₍ₛ₎ and filtered. The filtrate was concentrated to give the crude. The crude was dissolved in acetonitrile (2 mL) at 40° C. After being stirred at 40° C. for 1 hour, the reaction mixture was cooled to room temperature and filtered. The white solids were collected by filtration to give the title compound (150 mg, 100% purity, 86% yield). LC-MS (ESI): R_{T}=1.32 min, mass calcd. for C₂₇H₂₂ClF₅N₄O₅ 612.1, m/z found 613.0 [M+H]⁺.

Compound 28

(3R,7S)-2-(4-Chloro-3-(trifluoromethyl)benzoyl)-9-(4-(difluoromethoxy)benzyl)-N,3-dimethyl-10-oxo-1,2,3,4,7,8,9,10-octahydropyrido[4′,3′:3,4]pyrazolo[1,5-a]pyrazine-7-carboxamide

To a solution of (3R,7S)-2-(4-chloro-3-(trifluoromethyl)benzoyl)-9-(4-(difluoromethoxy)benzyl)-3-methyl-10-oxo-1,2,3,4,7,8,9,10-octahydropyrido[4′,3′:3,4]pyrazolo[1,5-a]pyrazine-7-carboxylic acid 28-3 (150 mg, 100% purity, 0.245 mmol) of N,N-dimethylformamide (5 mL) was added methylamine hydrochloride (50 mg, 0.741 mmol), 1H-benzo[d][1,2,3]triazol-1-ol (70 mg, 0.518 mmol), 1-(3-dimethylaminopropyl)-3-ethylcarbodiimide hydrochloride (100 mg, 0.522 mmol) and triethylamine (0.2 mL, 1.38 mmol) at 0° C. After being stirred at 0° C. for 2 hours, the mixture was added ethyl acetate (20 mL), washed with water (20 mL) twice, and then washed with brine (20 mL), dried over Na₂SO₄₍ₛ₎ and filtered. The filtrate was concentrated under reduced pressure to give crude. The crude was purified by C18 column (acetonitrile:water=5% to 100%) to give the title compound (70 mg, 98.4% purity, 45% yield) as yellow oil. LC-MS (ESI): R_{T}=1.63 min, mass calcd. for C₂₈H₂₅ClF₅N₅O₄ 625.2, m/z found 626.1 [M+H]⁺. ¹H NMR (400 MHz, CDCl₃) δ 7.78 (s, 1H), 7.60-7.52 (m, 2H), 7.31 (d, J=7.6 Hz, 2H), 7.09 (d, J=8.0 Hz, 2H), 6.49 (t, J=73.6 Hz, 1H), 6.25-5.38 (m, 2H), 5.03-4.42 (m, 5H), 4.19-4.15 (m, 1H), 3.87 (dd, J=12.0 and 5.2 Hz, 1H), 3.19-2.95 (m, 1H), 2.78-2.71 (m, 4H), 1.31 (d, J=6.8 Hz, 3H). ¹⁹F NMR (376 MHz, CDCl₃) δ −62.84, −80.98.

Compounds 29A and 29B

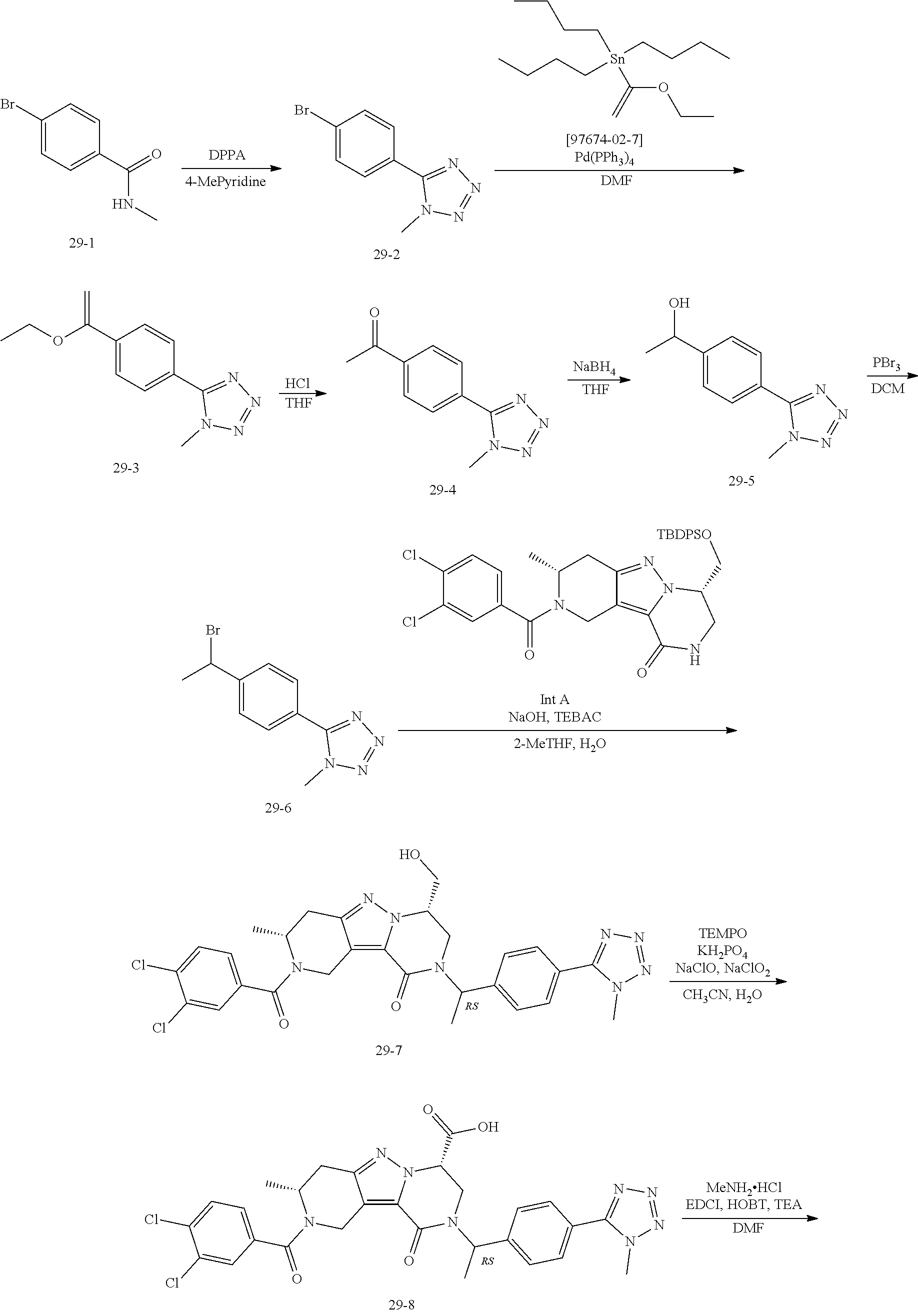

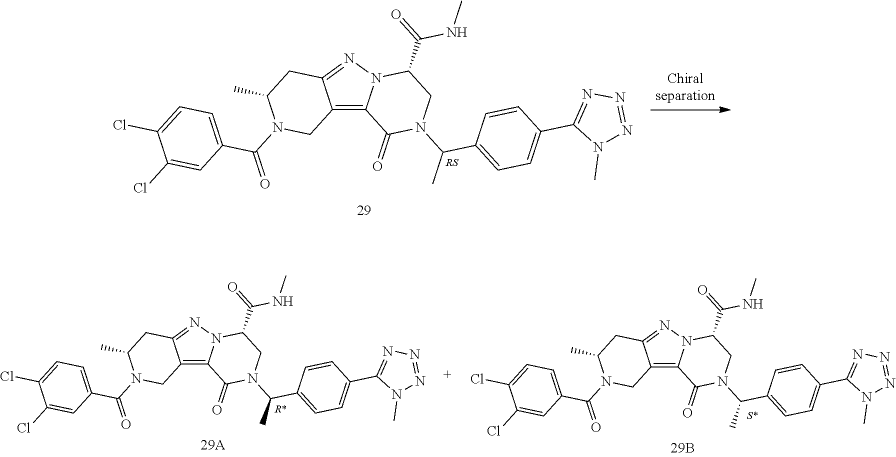 [SMILES: CNC(=O)[C@@H]1CN(C(C)c2ccc(-c3nnnn3C)cc2)C(=O)c2c3c(nn21)C[C@@H](C)N(C(=O)c1ccc(Cl)c(Cl)c1)C3 ; CNC(=O)[C@@H]1CN([C@H](C)c2ccc(-c3nnnn3C)cc2)C(=O)c2c3c(nn21)C[C@@H](C)N(C(=O)c1ccc(Cl)c(Cl)c1)C3 ; CNC(=O)[C@@H]1CN([C@@H](C)c2ccc(-c3nnnn3C)cc2)C(=O)c2c3c(nn21)C[C@@H](C)N(C(=O)c1ccc(Cl)c(Cl)c1)C3]

Intermediate 29-2

5-(4-Bromophenyl)-1-methyl-1H-tetrazole

To a solution of 4-bromo-N-methylbenzamide 29-1 (2.0 g, 9.34 mmol) in 4-methylpyridine (17 mL) was added diphenyl azidophosphate aqueous solution (10 mL, 46.4 mmol) at 25 TC. After being stirred at 149° C. for 21 hours, the reaction mixture was cooled down to room temperature. The solution was poured into 2 M hydrochloride aqueous solution (40 mL) and extracted with ethyl acetate (40 mL) twice. The organic layers were washed with saturated sodium bicarbonate aqueous solution (30 mL) and brine (30 mL), dried over Na₂SO₄₍ₛ₎ and filtered. The filtrate was concentrated under reduced pressure to give the crude compound, which was purified by C18 column (acetonitrile:water=30% to 55%) to give the title compound (1.23 g, 90% purity from ¹H NMR, 49.6% yield) as white solids. LC-MS (ESI): R_{T}=1.42 min, mass calcd. for C₈H₇BrN₄ 238.0, m/z found 239.0 [M+H]⁺. ¹H NMR (400 MHz, CDCl₃) δ 7.73 (dd, J=6.4 and 1.6 Hz, 2H), 7.64 (dd, J=4.4 and 2.0 Hz, 2H), 4.18 (s, 3H).

Intermediate 29-3

5-(4-(1-Ethoxyvinyl)phenyl)-1-methyl-1H-tetrazole

To a degassed solution of 5-(4-bromophenyl)-1-methyl-1H-tetrazole 29-2 (1.23 g, 90% purity, 4.63 mmol) and tributyl(1-ethoxyethenyl)stannane (2 mL, 5.92 mmol) in N,N-dimethylformamide (20 mL) was added tetrakis(triphenylphosphine) palladium (850 mg, 0.74 mmol). After being stirred at 80° C. overnight, the reaction mixture was cooled down to room temperature and treated with potassium fluoride aqueous solution (80 mL). After being stirred vigorously for 1 hour, the mixture was filtered with kieselguhr. The filtrate was diluted with ethyl acetate (50 mL), washed with saturated sodium bicarbonate solution (50 mL), brine (50 mL), dried over Na₂SO₄₍ₛ₎ and filtered. The filtrate was concentrated to give the crude (1.1 g, 67.5% purity, 69.6% yield) as yellow oil. LC-MS (ESI): R_{T}=1.084 min, mass calcd. for C₂H₁₄N₄O 230.0, m/z found 231.2 [M+H]⁺.

Intermediate 29-4

1-(4-(1-Methyl-1H-tetrazol-5-yl)phenyl)ethan-1-one

The mixture of 5-(4-(1-ethoxyvinyl)phenyl)-1-methyl-1H-tetrazole 29-3 (1.1 g, 67.5% purity, 3.23 mmol) in 4 M hydrochloride aqueous solution (10 mL, 40 mmol) was stirred at room temperature for 2 hours. Then it was adjusted to pH−7 with 2 M sodium hydroxide aqueous solution, extracted with dichloromethane (30 mL) twice. The combined organic layers were washed with brine (30 mL) and dried over Na₂SO₄₍ₛ₎ and filtered. The filtrate was concentrated to give the crude, which was purified by C18 column (acetonitrile:water=5% to 90%) to give the title compound (386 mg, 90% purity from ¹H NMR, 53.3% yield) as yellow solids. LC-MS (ESI): R_{T}=1.12 min, mass calcd. for C₁₀H₁₀N₄O 202.2, m/z found 203.1 [M+H]⁺. 4H NMR (400 MHz, CDCl₃) δ 8.15 (d, J=8.4 Hz, 2H), 7.89 (d, J=8.8 Hz, 2H), 4.22 (s, 3H), 2.69 (s, 3H).

Intermediate 29-5

1-(4-(1-Methyl-1H-tetrazol-5-yl)phenyl)ethan-1-ol

To a solution of 1-(4-(1-methyl-1H-tetrazol-5-yl)phenyl)ethan-1-one 29-4 (286 mg, 90% purity, 1.27 mmol) in tetrahydrofuran (1.5 mL) and methanol (0.5 mL) was added sodium borohydride (100 mg, 2.64 mmol) at 0° C. After being stirred at 0° C. for 1 hour, the mixture was quenched with water (20 mL). The organic phase was separated and the aqueous phase was extracted with ethyl acetate (30 mL) for three times. The combined organic layers were washed with brine (30 mL), dried over Na₂SO₄₍ₛ₎ and filtered. The filtrate was concentrated under reduced pressure to get the title compound (300 mg, 80% purity from ¹H NMR, 92.3% yield) as yellow oil. LC-MS (ESI): R_{T}=1.21 min, mass calcd. for C₁₀H₁₂N₄O 204.2, m/z found 205.1 [M+H]⁺. ¹H NMR (400 MHz, CDCl₃) δ 7.64 (d, J=8.4 Hz, 2H), 7.51 (d, J=8.0 Hz, 2H), 4.94 (q, J=8.0 Hz, 1H), 4.10 (s, 3H), 1.47 (d, J=6.0 Hz, 3H).

Intermediate 29-6

5-(4-(1-bromoethyl)phenyl)-1-methyl-1H-tetrazole

To a solution of 1-(4-(1-Methyl-1H-tetrazol-5-yl)phenyl)ethan-1-ol 29-5 (300 mg, 80% purity, 1.18 mmol) in dichloromethane (1 mL) was added tribromophosphine (260 mg, 0.96 mmol) at 0° C. After being stirred at 0° C. for 1 hour, the mixture was quenched by the addition of water. The organic phase was washed with saturated aqueous sodium bicarbonate, dried over Na₂SO₄₍ₛ₎ and filtered. The filtrate was concentrated under reduced pressure to give the residue, which was purified by silica gel column chromatography (petroleum ether:ethyl acetate=3:1) to give the title compound (138 mg, 85% purity by ¹H NMR, 37.4% yield) as white solids. LC-MS (ESI): R_{T}=1.51 min, mass calcd. for C₁₀H₁₁BrN₄ 266.0, m/z found 267.0 [M+H]⁺. ¹H NMR (400 MHz, CDCl₃) δ 7.75-7.73 (m, 2H), 7.66-7.64 (m, 2H), 5.28-5.22 (m, 1H), 4.20 (d, J=3.6 Hz, 3H), 2.11-2.08 (m, 3H).

Intermediate 29-7

(3R,7S)-2-(3,4-Dichlorobenzoyl)-7-(hydroxymethyl)-3-methyl-9-(1-(4-(1-methyl-1H-tetrazol-5-yl)phenyl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichloro benzoyl)-3-methyl-1,2,3,4,8,9-hexahydropyrido[4′,3′:3,4]pyrazolo[1,5-a]pyrazin-10(7H)-one Int A (250 mg, 93% purity, 0.36 mmol) and 5-(4-(1-bromoethyl)phenyl)-1-methyl-1H-tetrazole 29-6 (138 mg, 85% purity, 0.44 mmol) in 2-methyltetrahydrofuran (4 mL) was added benzyltriethyl ammonium (15 mg, 0.07 mmol) and 50% wt. sodium hydroxide in water (4 mL) slowly at 0° C. After being stirred at room temperature overnight, the reaction mixture was diluted with water (30 mL) and extracted with ethyl acetate (30 mL) twice. The combined organic layers were washed with brine (30 mL) twice, dried over Na₂SO₄₍ₛ₎ and filtered. The filtrate was concentrated to give the title compound (200 mg, 88.1% purity from LCMS, 82.4% yield) as yellow solids. LC-MS (ESI): R_{T}=1.49 min, mass calcd. for C₂₈H₂₈Cl₂N₈O₃ 594.1, m/z found 595.2 [M+H]⁺.

Intermediate 29-8

(3R,7S)-2-(3,4-Dichlorobenzoyl)-3-methyl-9-(1-(4-(1-methyl-1H-tetrazol-5-yl)phenyl)ethyl)-10-oxo-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-7-(hydroxymethyl)-3-methyl-9-(1-(4-(1-methyl-1H-tetrazol-5-yl)phenyl)ethyl)-1,2,3,4,8,9-hexahydropyrido[4′,3′:3,4]pyrazolo[1,5-a]pyrazin-10(7H)-one 29-7 (200 mg, 88% purity, 0.30 mmol) in acetonitrile (2 mL) was added 2,2,6,6-tetramethylpiperidinooxy (95 mg, 0.61 mmol), sodium chlorite (68 mg, 80% purity, 0.60 mmol), saturated potassium dihydrogenphosphate aqueous solution (2 mL) and sodium hypochlorite aqueous solution (0.35 mL, 10% purity, 0.59 mmol) at 0° C. After being stirred at 0° C. for 2 hours, the mixture was diluted with sodium sulfite saturated solution (20 mL) and extracted with ethyl acetate (30 mL) twice. The combined organic layers were washed with brine (30 mL), dried over Na₂SO₄₍ₛ₎ and filtered. The filtrate was concentrated under reduced pressure to give title compound (200 mg, 85.6% purity from LCMS, 95.0% yield) as yellow solids. LC-MS (ESI): R_{T}=1.27 min, mass calcd. for C₂₈H₂₆Cl₂N₈O₄ 608.2, m/z found 609.1 [M+H]⁺.

Compound 29

(3R,7S)-2-(3,4-Dichlorobenzoyl)-N,3-dimethyl-9-(1-(4-(1-methyl-1H-tetrazol-5-yl)phenyl)ethyl)-10-oxo-1,2,3,4,7,8,9,10-octahydropyrido[4′,3′:3,4]pyrazolo[1,5-a]pyrazine-7-carboxamide

To a mixture of (3R,7S)-2-(3,4-dichlorobenzoyl)-3-methyl-9-(1-(4-(1-methyl-1H-tetrazol-5-yl)phenyl)ethyl)-10-oxo-1,2,3,4,7,8,9,10-octahydropyrido[4′,3′:3,4]pyrazolo[1,5-a]pyrazine-7-carboxylic acid 29-8 (200 mg, 85.6% purity, 0.28 mmol), methanamine hydrochloride (45 mg, 0.67 mmol), 1-ethyl-(3-(3-dimethylamino)propyl)-carbodiimide hydrochloride (115 mg, 0.60 mmol) and benzotriazol-1-ol (95 mg, 0.70 mmol) in N,N-dimethylformamide (2 mL) was added triethylamine (0.25 mL, 1.78 mmol) at 0° C. After being stirred at 0° C. under nitrogen atmosphere for 2 hours, the mixture was acidified to pH=6 with 0.5 M hydrochloride aqueous solution and extracted with ethyl acetate (30 mL) twice. The combined organic layers were washed with water (30 mL) for three times and brine (30 mL), dried over Na₂SO₄₍ₛ₎ and filtered. The filtrate was concentrated under reduced pressure to give a residue, which was purified by C18 column (acetonitrile:water=55% to 75%) to give the title compound (120 mg, 90% purity from ¹H NMR, 61.7% yield) as white solids. LC-MS (ESI): R_{T}=1.49 min, mass calcd. for C₂₉H₂₉Cl₂N₉O₃ 621.2, m/z found 622.1 [M+H]⁺. ¹H NMR (400 MHz, CDCl₃) δ 7.77-7.71 (m, 2H), 7.58-7.50 (m, 4H), 7.28-7.26 (m, 1H), 6.12-5.93 (m, 2H), 5.29-4.87 (m, 2H), 4.46-4.12 (m, 5H), 3.96-3.86 (m, 1H), 3.48-3.42 (m, 1H), 3.13-2.97 (m, 1H), 2.82-2.68 (m, 4H), 1.65 (d, J=7.2 Hz, 3H), 1.31 (d, J=6.8 Hz, 3H).

Compounds 29A and 29B

(3R,7S)-2-(3,4-Dichlorobenzoyl)-N,3-dimethyl-9-((R*)-1-(4-(1-methyl-1H-tetrazol-5-yl)phenyl)ethyl)-10-oxo-1,2,3,4,7,8,9,10-octahydropyrido[4′,3′:3,4]pyrazolo[1,5-a]pyrazine-7-carboxamide (29A), and (3R,7S)-2-(3,4-dichlorobenzoyl)-N,3-dimethyl-9-((S*)-1-(4-(1-methyl-1H-tetrazol-5-yl)phenyl)ethyl)-10-oxo-1,2,3,4,7,8,9,10-octahydropyrido[4′,3′:3,4]pyrazolo[1,5-a]pyrazine-7-carboxamide (29B)

A racemic mixture of (3R,7S)-2-(3,4-dichlorobenzoyl)-N,3-dimethyl-9-(1-(4-(1-methyl-1H-tetrazol-5-yl)phenyl)ethyl)-10-oxo-1,2,3,4,7,8,9,10-octahydropyrido[4′,3′:3,4]pyrazolo[1,5-a]pyrazine-7-carboxamide 29 (120 mg, 90% purity, 0.17 mmol) was separated by chiral Prep. HPLC (separation conditon: Column: Chiralpak IB N-5 5 μm 20*250 mm; Mobile Phase: 100% ACN at 25 mL/min; Temp: 30° C.; Wavelength: 254 nm) to give the title compounds 29A (30 mg, 99.8% purity from LCMS, 27.7% yield, 100% stereopure) and 29B (40 mg, 99.8% purity from LCMS, 37.0% yield, 99.3% stereopure) as white solids.

Compound 29A

LC-MS (ESI): R_{T}=3.449 min, mass calcd. for C₂₉H₂₉Cl₂N₉O₃ 621.2, m/z found 622.2 [M+H]⁺. Chiral analysis (Column: Chiralpak IB N-5 5 μm 4.6*250 mm; Mobile Phase: 100% ACN at 1 mL/min; Col. Temp: 30° C.; Wavelength: 254 nm; Rt=5.644 min). ¹H NMR (400 MHz, CDCl₃) δ 7.72 (d, J=8.4 Hz, 2H), 7.54-7.50 (m, 4H), 7.28-7.26 (m, 1H), 5.98-5.48 (m, 3H), 4.88-4.42 (m, 3H), 4.19 (s, 3H), 3.92 (d, J=4.0 Hz, 2H), 3.17-2.97 (m, 1H), 2.74-2.67 (m, 4H), 1.66 (d, J=6.8 Hz, 3H), 1.31 (d, J=6.8 Hz, 3H).

Compound 29B

LC-MS (ESI): R_{T}=3.641 min, mass calcd. for C₂₉H₂₉Cl₂N₉O₃ 621.2, m/z found 622.2 [M+H]⁺. Chiral analysis (Column: Chiralpak IB N-5 5 μm 4.6*250 mm; Mobile Phase: 100% ACN at 1 mL/min; Col. Temp: 30° C.; Wavelength: 254 nm; Rt=9.617 min). ¹H NMR (400 MHz, CDCl₃) δ 7.75 (d, J=8.0 Hz, 2H), 7.59-7.52 (m, 4H), 7.28-7.26 (m, 1H), 6.10-5.45 (m, 3H), 4.86-4.38 (m, 3H), 4.18-4.13 (m, 4H), 3.46 (dd, J=13.2, 4.8 Hz, 1H), 3.12-2.98 (m, 1H), 2.81 (d, J=4.8 Hz, 3H), 2.74-2.70 (m, 1H), 1.65 (d, J=6.8 Hz, 3H), 1.31 (d, J=6.8 Hz, 3H).

Compounds 30A and 30B

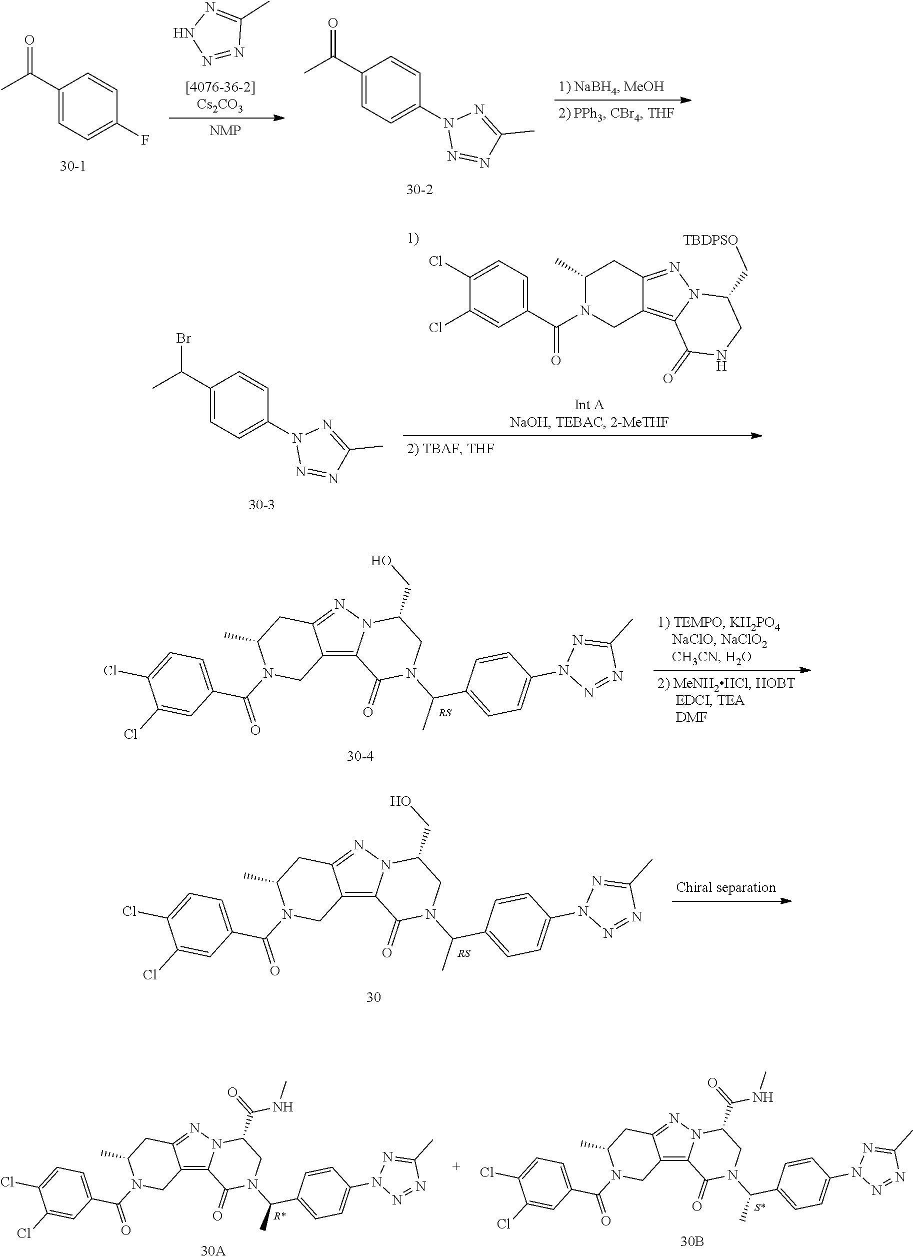

Intermediate 30-2

1-(4-(5-Methyl-2H-tetrazol-2-yl)phenyl)ethan-1-one

A solution of 1-(4-fluorophenyl)ethan-1-one 30-1 (3 g, 21.7 mmol), 5-methyl-2H-tetrazole (2.4 g, 28.5 mmol) and cesium carbonate (14 g, 43.0 mmol) in 1-methylpyrrolidin-2-one (30 mL) was stirred at 130° C. for 48 hours. Then, the mixture was diluted with water (100 mL) and extracted with ethyl acetate (50 mL) twice. The combined organic layers were washed with water (50 mL) for three times and brine (50 mL), dried over Na₂SO₄₍ₛ₎ and filtered. The crude was purified by C18 column (acetonitrile:water=30% to 80%) to give the title compound (400 mg, 90% purity from ¹H NMR, 8% yield) as yellow oil. ¹H NMR (400 MHz, DMSO-d₆) δ 8.14-8.21 (m, 4H), 2.66 (s, 3H), 2.62 (s, 3H).

Intermediate 30-3

(4-(1-Bromoethyl)phenyl)-5-methyl-2H-tetrazole

To a solution of 1-(4-(5-methyl-2H-tetrazol-2-yl)phenyl)ethan-1-one 30-2 (400 mg, 90% purity, 1.78 mmol) in methanol (2 mL) and tetrahydrofuran (4 mL) was added sodium borohydride (27 mg, 0.714 mmol) at 0° C. After being stirred at 0° C. for 0.5 hour, the reaction mixture was quenched with saturated ammonium chloride aqueous solution (2 mL), dried over Na₂SO₄₍ₛ₎ and filtered. The filtrate was concentrated under reduced pressure to give a residue, which was diluted with tetrahydrofuran (6 mL), then triphenylphosphine (607 mg, 2.31 mmol) and carbon tetrabromide (666 mg, 2.01 mmol) was added into the solution. After being stirred at 0° C. for 2 hours under nitrogen atmosphere, the reaction mixture was concentrated and purified by silica gel column chromatography (petroleum ether:ethyl acetate=6:1) to give the title compound (400 mg, 90% purity from ¹H NMR, 76% yield) as yellow oil. ¹H NMR (400 MHz, DMSO-d₆) δ 8.06 (d, J=8.8 Hz, 2H), 7.79 (d, J=8.8 Hz, 2H), 5.62 (q, J=6.8 Hz, 1H), 2.59 (s, 3H), 2.03 (d, J=6.8 Hz, 3H).

Intermediate 30-4

(3R,7S)-2-(3,4-Dichlorobenzoyl)-7-(hydroxymethyl)-3-methyl-9-(1-(4-(5-methyl-2H-tetrazol-2-yl)phenyl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichloro benzoyl)-3-methyl-1,2,3,4,8,9-hexahydropyrido[4′,3′:3,4]pyrazolo[1,5-a]pyrazin-10(7H)-one Int A (750 mg, 100% purity, 1.16 mmol) and 2-(4-(1-bromoethyl)phenyl)-5-methyl-2H-tetrazole 30-3 (400 mg, 90% purity, 1.35 mmol) in 2-methyltetrahydrofuran (5 mL) was added 50% wt. sodium hydroxide aqueous solution (5 mL) and benzyl triethylammonium chloride (53 mg, 0.232 mmol). After being stirred at 20° C. for 2 hours, the mixture was added into water (50 mL) and extracted with dichloromethane (50 mL) twice. The combined organic layers were washed with water (50 mL), brine (50 mL), dried over Na₂SO₄₍ₛ₎ and filtered. The filtrate was concentrated under reduced pressure to give a residue, which was diluted in tetrahydrofuran (20 mL). 1 M tetrabutylammonium fluoride in tetrahydrofuran (2.5 mL, 2.5 mmol) was added into the mixture. After being stirred at 20° C. for 3 hours. The reaction mixture was diluted with water (30 mL) and extracted with ethyl acetate (30 mL) twice. The combined organic layers were washed with water (30 mL) for three times, and brine (30 mL), dried over Na₂SO₄₍ₛ₎ and filtered. The filtrate was concentrated to give a residue, which was purified by C18 column (acetonitrile:water=5% to 95%) to give the title compound (450 mg, 100% purity from LCMS, 65% yield) as white solids. LC-MS (ESI): R_{T}=1.60 min, mass calcd. for C₂₈H₂₈Cl₂N₈O₃ 594.2 mz found 595.1[M+H]⁺.

Compound 30

(3R,7S)-2-(3,4-Dichlorobenzoyl)-N,3-dimethyl-9-(1-(4-(5-methyl-2H-tetrazol-2-yl)phenyl)ethyl)-10-oxo-1,2,3,4,7,8,9,10-octahydropyrido[4′,3′:3,4]pyrazolo[1,5-a]pyrazine-7-carboxamide

To a solution of (3R,7S)-2-(3,4-dichlorobenzoyl)-7-(hydroxymethyl)-3-methyl-9-(1-(4-(5-methyl-2H-tetrazol-2-yl)phenyl)ethyl)-1,2,3,4,8,9-hexahydropyrido[4′,3′:3,4]pyrazolo[1,5-a]pyrazin-10(7H)-one 30-4 (300 mg, 100% purity, 0.504 mmol) in saturated potassium phosphate monobasic aqueous solution (4.5 mL) and acetonitrile (4.5 mL) were added sodium chlorite (114 mg, 80% purity, 1.01 mmol), 2,2,6,6-tetramethylpiperidinooxy (158 mg, 1.01 mmol) and 10% sodium hypochlorite aqueous solution (0.6 mL, 1.01 mmol) at 0° C. After being stirring at 0° C. for 4 hours, the reaction mixture was diluted with 0.1 M hydrochloride aqueous solution (40 mL) and extracted with ethyl acetate (40 mL) twice. The combined organic layers were washed with brine (40 mL) and dried over Na₂SO₄₍ₛ₎ and filtered. The filtrate was concentrated to give a residue, which was washed with (petroleum ether:ethyl acetate, 3 mL:1 mL) and filtered to give a residue, which was diluted with N,N-dimethylformamide (3 mL). Then methylamine hydrochloride (61 mg, 0.903 mmol), benzotriazol-1-ol (97 mg, 0.718 mmol), 1-(3-dimethylaminopropyl)-3-ethylcarbodiimide hydrochloride (139 mg, 0.725 mmol) and triethylamine (257 mg, 2.54 mmol) were added into the solution at 0° C. After being stirred at 0° C. for 2 hours, the mixture was acidified to pH=6 with 0.05 M hydrochloride aqueous solution and extracted with ethyl acetate (60 mL) twice. The combined organic layers were washed with water (60 mL) for three times and brine (20 mL), dried over Na₂SO₄ (s) and filtered. The filtrate was concentrated under reduced pressure to give a residue, which was purified by C18 column (acetonitrile:water=55% to 60%) to give the title compound (150 mg, 100% purity, 67% yield) as white solids. LC-MS (ESI): R_{T}=1.60 min, mass calcd. for C₂₉H₂₉Cl₂N₉O₃ 621.2 mz found 622.1[M+H]⁺.

Compounds 30A and 30B

(3R,7S)-2-(3,4-Dichlorobenzoyl)-N,3-dimethyl-9-((R*)-1-(4-(5-methyl-2H-tetrazol-2-yl)phenyl)ethyl)-10-oxo-1,2,3,4,7,8,9,10-octahydropyrido[4′,3′:3,4]pyrazolo[1,5-a]pyrazine-7-carboxamide (30A), and (3R,7S)-2-(3,4-dichlorobenzoyl)-N,3-dimethyl-9-((S*)-1-(4-(5-methyl-2H-tetrazol-2-yl)phenyl)ethyl)-10-oxo-1,2,3,4,7,8,9,10-octahydropyrido[4′,3′:3,4]pyrazolo[1,5-a]pyrazine-7-carboxamide (30B)

A racemic mixture of (3R,7S)-2-(3,4-dichlorobenzoyl)-N,3-dimethyl-9-(1-(4-(5-methyl-2H-tetrazol-2-yl)phenyl)ethyl)-10-oxo-1,2,3,4,7,8,9,10-octahydropyrido[4′,3′:3,4]pyrazolo[1,5-a]pyrazine-7-carboxamide 30 (150 mg, 100% purity, 0.241 mmol) was separated by chiral HPLC (separation condition: Column Chiralpak IE 5 μm 20*250 mm; Mobile Phase: CAN:IPA=70:30 at 15 mL/min; Temp: 30° C.; Wavelength: 254 nm) to give the title compound 30A (31.1 mg, 98.3% purity, 20% yield, 99.98% stereopure) as white solids and compound 30B (52.0 mg, 99.2% purity, 34% yield, 99.9% stereopure) as white solids.

Compound 30A

LC-MS (ESI): R_{T}=3.700 min, mass calcd. for C₂₉H₂₉Cl₂N₉O₃ 621.2, mz found 622.1[M+H]⁺. Chiral analysis (Column: Superchiral IE 5 μm 4.6*250 mm; Mobile Phase: ACN:IPA=70:30 at 1 mL/min; Temp: 30° C.; Wavelength: 254 nm; Rt=8.617 min), ¹H NMR (400 MHz, CDCl₃) δ 8.08 (d, J=8.8 Hz, 2H), 7.54-7.46 (m, 4H), 7.28-7.26 (m, 1H), 6.01-5.47 (m, 3H), 4.88-4.47 (m, 3H), 3.97-3.88 (m, 2H), 3.13-2.97 (m, 1H), 2.74-2.68 (m, 4H), 2.64 (s, 3H), 1.65 (d, J=7.2 Hz, 3H), 1.31 (d, J=6.8 Hz, 3H).

Compound 30B

LC-MS (ESI): R_{T}=3.772 min, mass calcd. for C₂₉H₂₉Cl₂N₉O₃ 621.2, mz found 622.1[M+H]⁺.

Chiral analysis (Column: Superchiral IE 5 μm 4.6*250 mm; Mobile Phase: ACN:IPA=70:30 at 1 mL/min; Temp: 30° C.; Wavelength: 254 nm; Rt=10.642 min). ¹H NMR (400 MHz, CDCl₃) δ 8.08 (d, J=8.4 Hz, 2H), 7.54-7.52 (m, 4H), 7.28-7.27 (m, 1H), 6.08-5.39 (m, 3H), 4.85-4.38 (m, 3H), 4.14-4.10 (m, 1H), 3.46-3.41 (m, 1H), 3.13-2.98 (m, 1H), 2.80 (d, J=4.8 Hz, 3H), 2.74-2.68 (m, 1H), 2.64 (s, 3H), 1.64 (d, J=7.2 Hz, 3H), 1.31 (d, J=6.8 Hz, 3H).

Compounds 31A and 31B

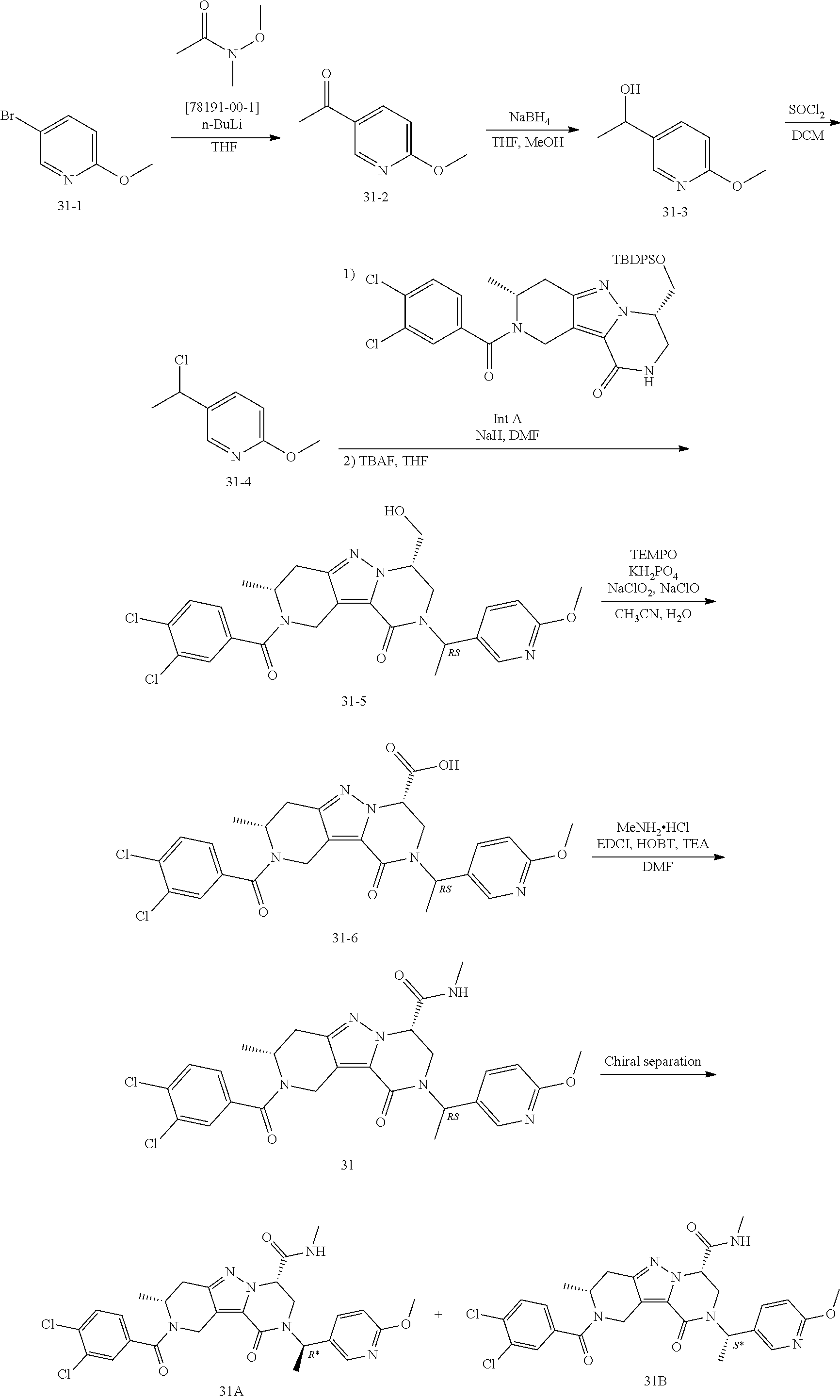

Intermediate 31-2

1-(6-Methoxypyridin-3-yl)ethanone

To a solution of 5-bromo-2-methoxypyridine 31-1 (8 g, 42.5 mmol) in tetrahydrofuran (80 mL) was added dropwise 2.5 M n-butyllithium in hexane (20 mL, 50 mmol) at −78° C. under nitrogen atmosphere. After being stirred for 0.5 hour at −78° C., the mixture was added N-methoxy-N-methylacetamide (9 mL, 84.7 mmol) and the mixture was kept at −78° C. for 1 hour. After being stirred at room temperature for 2 hours, the mixture was quenched with saturated ammonium chloride aqueous solution, extracted with ethyl acetate (80 mL) for three times. The organic layers were combined, washed with brine (80 mL), dried over Na₂SO₄₍ₛ₎ and filtered. The filtrate was concentrated to get a residue, which was purified by silica gel column chromatography (petroleum ether:ethyl acetate=40:1 to 10:1) to give the title compound (4.7 g, 100% purity from LCMS, 69% yield) as white solids. LC-MS (ESI): R_{T}=1.30 min, mass calcd. for C₈H₉NO₂ 151.1, m/z found 152.2 [M+H]⁺.

Intermediate 31-3

1-(6-Methoxypyridin-3-yl)ethanol

To a solution of 1-(6-methoxypyridin-3-yl)ethan-1-one 31-2 (2 g, 95% purity, 12.6 mmol) in tetrahydrofuran (20 mL) were added sodium borohydride (550 mg, 14.5 mmol) and methanol (5 mL). After being stirred at 0° C. for 1 hour, the mixture was quenched with saturated ammonium chloride aqueous solution, extracted with ethyl acetate (20 mL) for three times. The organic layers were combined, washed with brine (10 mL), dried over Na₂SO₄₍ₛ₎ and filtered. The filtrate was concentrated to get the title compound (1.9 g, 90% purity from NMR, 89% yield) as colorless oil. ¹H NMR (300 MHz, DMSO-d₆) δ 8.09 (d, J=2.4 Hz, 1H), 7.67 (dd, J=8.4 and 2.4 Hz, 1H), 6.76 (d, J=8.8 Hz, 1H), 5.16 (d, J=4.4 Hz, 1H), 4.74-4.68 (m, 1H), 3.82 (s, 3H), 1.32 (d, J=6.4 Hz, 3H).

Intermediate 31-4

5-(1-Chloroethyl)-2-methoxypyridine

To a solution of 1-(6-methoxypyridin-3-yl)ethanol 31-3 (1.9 g, 90% purity, 11.2 mmol) in dichloromethane (20 mL) was added thionyl chloride (0.9 mL, 12.4 mmol) at 0° C. After being stirred at 40° C. for 2 hours, the solvent was removed. The mixture was basified to pH 7-8 with saturated sodium bicarbonate aqueous solution, extracted with dichloromethane (20 mL) for twice. The organic layers were washed with brine (10 mL), dried over Na₂SO₄₍ₛ₎, filtered and concentrated to give the title compound (1.4 g, 95% purity from ¹H NMR, 69% yield) as yellow oil. ¹H NMR (300 MHz, CDCl₃) δ 8.15 (d, J=2.1 Hz, 1H), 7.68 (dd, J=8.7, 2.7 Hz, 1H), 6.76 (d, J=8.7 Hz, 1H), 5.09 (q, J=6.6 Hz, 1H), 3.94 (s, 3H), 1.84 (d, J=6.6 Hz, 3H).

Intermediate 31-5

(3R,7S)-2-(3,4-Dichlorobenzoyl)-7-(hydroxymethyl)-9-(1-(6-methoxypyridin-3-yl)ethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (1.7 g, 83% purity, 2.18 mmol) in N,N-dimethylformamide (15 mL) was added 60% wt. Sodium hydride in mineral oil (170 mg, 4.25 mmol) at 0° C. After being stirred at 0° C. for 30 minutes, the reaction mixture was added 5-(1-chloroethyl)-2-methoxypyridine 31-4 (591 mg, 95% purity, 3.27 mmol) at 0° C. After being stirred at room temperature (25° C.) overnight, the reaction mixture was quenched with brine (40 mL), extracted with ethyl acetate (20 mL) for three times, dried over Na₂SO₄₍ₛ₎, filtered and concentrated to give the crude product. To the crude product in tetrahydrofuran (3 mL) was added 1 M tetrabutylammonium fluoride in tetrahydrofuran (1.1 mL, 1.1 mmol) at 0° C. After being stirred at room temperature for 1 hour, the mixture was concentrated and purified by silica gel column chromatography (petroleum ether:acetone=4:1 to 2:1) to give the title compound (700 mg, 100% purity from LCMS, 59% yield) as white solids. LC-MS (ESI): R_{T}=1.54 min, mass calcd. for C₂₆H₂₇Cl₂N₅O₄ 543.1, m/z found 544.3 [M+H]⁺.

Intermediate 31-6

(3R,7S)-2-(3,4-Dichlorobenzoyl)-9-(1-(6-methoxypyridin-3-yl)ethyl)-3-methyl-10-oxo-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-7-(hydroxymethyl)-9-(1-(6-methoxypyridin-3-yl)ethyl)-3-methyl-1,2,3,4,8,9-hexahydropyrido[4′,3′:3,4]pyrazolo[1,5-a]pyrazin-10(7H)-one 31-5 (700 mg, 100% purity, 1.29 mmol) in acetonitrile (7 mL) were added saturated potassium dihydrogen phosphate aqueous solution (7 mL), 2,2,6,6-tetramethylpiperidinooxy (448 mg, 2.87 mmol), sodium chlorite (340 mg, 3.01 mmol) and dropwise 5.5% sodium hypochlorite aqueous solution (1.8 mL, 3.02 mmol) at 0° C. The reaction was allowed to slowly return to room temperature. After being stirred at room temperature for 4 hours, the reaction mixture was quenched with saturated sodium thiosulfate (14 mL), acidized with 1 M hydrochloride aqueous solution to pH 4-5, extracted with ethyl acetate (10 mL) for three times. The combined organic layers were washed with brine (10 mL), dried over Na₂SO₄₍ₛ₎, and filtered. The filtrate was concentrated and purified by triturated with petroleum ether:ethyl acetate=5:1 (10 mL) at room temperature. After being stirred for 30 minutes, the mixture was filtered to give the title compound (700 mg, 78% purity from LCMS, 76% yield) as white solids. LC-MS (ESI): R_{T}=1.163 min, mass calcd. for C₂₆H₂₅Cl₂N₅O₅ 557.1, m/z found 558.0 [M+H]⁺.

Compound 31

(3R,7S)-2-(3,4-Dichlorobenzoyl)-9-(1-(6-methoxypyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide

To a solution of (3R,7S)-2-(3,4-dichlorobenzoyl)-9-(1-(6-methoxypyridin-3-yl)ethyl)-3-methyl-10-oxo-1,2,3,4,7,8,9,10-octahydropyrido[4′,3′:3,4]pyrazolo[1,5-a]pyrazine-7-carboxylic acid 31-6 (800 mg, 78% purity, 1.12 mmol) in N,N-dimethylformamide (9 mL) were added 1-(3-dimethylaminopropyl)-3-ethylcarbodiimide hydrochloride (472 mg, 2.46 mmol), 1-hydroxybenzotriazole (312 mg, 2.31 mmol), methylamine hydrochloride (192 mg, 2.84 mmol) and dropwise triethylamine (1.1 mL, 7.91 mmol) in N,N-dimethylformamide (9 mL) at 0° C. After being stirred at 0° C. for 10 minutes, the mixture was added water (50 mL), extracted with ethyl acetate (20 mL) for three times. The organic layers were washed with brine (20 mL), dried over Na₂SO₄₍ₛ₎, filtered. The filtrate was concentrated and purified by C18 column (acetonitrile:water=5% to 65%) to give the title compound (600 mg, 96% purity from LCMS, 90% yield) as pale yellow solids. LC-MS (ESI): R_{T}=1.50 min, mass calcd. for C₂₇H₂₈Cl₂N₆O₄ 570.2, m/z found 571.3 [M+H]⁺.

Compounds 31A and 31B

(3R,7S)-2-(3,4-Dichlorobenzoyl)-9-((R*)-1-(6-methoxypyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide (31A), and (3R,7S)-2-(3,4-dichlorobenzoyl)-9-((S*)-1-(6-methoxypyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide (31B)

A racemate of (3R,7S)-2-(3,4-dichlorobenzoyl)-9-(1-(6-methoxypyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide 31 (600 mg, 96% purity, 1.01 mmol) was purified by chiral HPLC (separation condition: Column: Chiralpak IB N-5 5 μm 30*250 mm; Mobile Phase: ACN-100 at 25 mL/min; Temp: 30° C.; Wavelength: 214 nm) to afford the title compounds 31A (233 mg, 98.2% purity from QC, 40% yield, 100% stereopure) as white solids and 31B (355 mg, 99.7% purity from QC, 61% yield, 99.9% stereopure) as white solids.

Compound 31A

LC-MS (ESI): R_{T}=8.526 min, mass calcd. for C₂₇H₂₈Cl₂N₆O₄ 570.2, m/z found 571.1 [M+H]⁺. Chiral analysis (Column: Chiralpak IB N-5 5 μm 4.6*250 mm; Mobile Phase: ACN-100 at 1 mL/min; Temp: 30° C.; Wavelength: 254 nm; Rt=7.707 min). ¹H NMR (400 MHz, DMSO-d₆) δ 8.12-7.99 (m, 1H), 7.81-7.74 (m, 3H), 7.56-7.45 (m, 2H), 6.80-6.78 (m, 1H), 5.82-4.96 (m, 3H), 4.68-3.91 (m, 3H), 3.84 (s, 3H), 3.43-3.32 (m, 1H), 2.99-2.82 (m, 1H), 2.67-2.50 (m, 1H), 2.37 (d, J=4.0 Hz, 3H), 1.50-1.17 (m, 6H).

Compound 31B

LC-MS (ESI): R_{T}=8.819 min, mass calcd. for C₂₇H₂₈Cl₂N₆O₄ 570.2, m/z found 571.1 [M+H]⁺. Chiral analysis (Column: Chiralpak IB N-5 5 μm 4.6*250 mm; Mobile Phase: ACN-100 at 1 mL/min; Temp: 30° C.; Wavelength: 254 nm; Rt=10.316 min). ¹H NMR (400 MHz, DMSO-d₆) δ 8.21-8.02 (m, 2H), 7.76-7.44 (m, 4H), 6.87-6.73 (m, 1H), 5.85-4.94 (m, 3H), 4.64-4.07 (m, 2H), 3.84 (s, 3H), 3.76-3.47 (m, 2H), 2.96-2.84 (m, 1H), 2.63 (d, J=4.4 Hz, 3H), 2.58-2.51 (m, 1H), 1.47-1.12 (m, 6H).

Compounds 32A and 32B

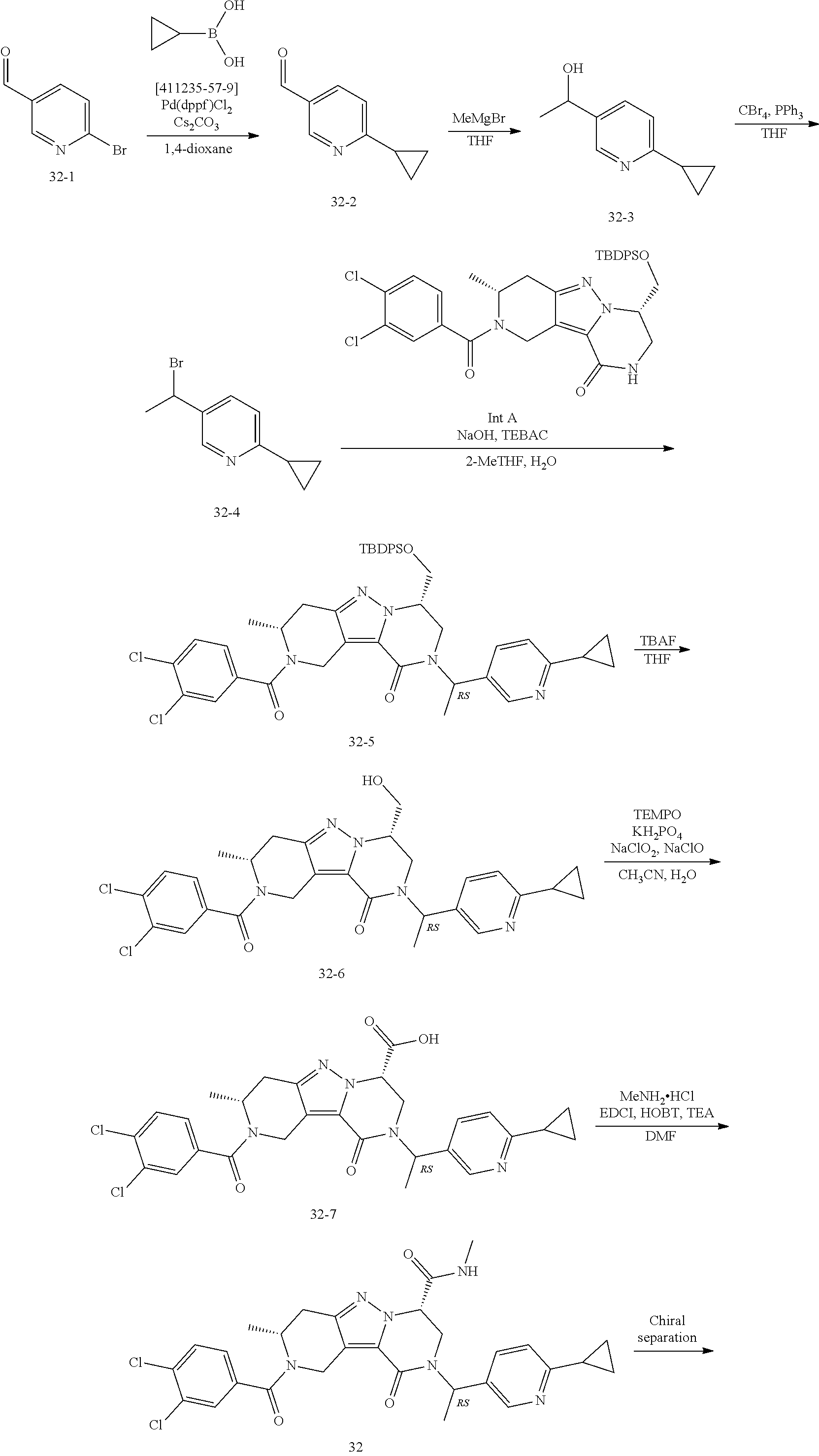

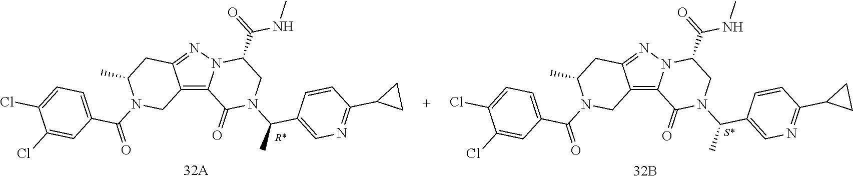 [SMILES: CNC(=O)[C@@H]1CN([C@H](C)c2ccc(C3CC3)nc2)C(=O)c2c3c(nn21)C[C@@H](C)N(C(=O)c1ccc(Cl)c(Cl)c1)C3 ; CNC(=O)[C@@H]1CN([C@@H](C)c2ccc(C3CC3)nc2)C(=O)c2c3c(nn21)C[C@@H](C)N(C(=O)c1ccc(Cl)c(Cl)c1)C3]

Intermediate 32-2

6-Cyclopropylnicotinaldehyde

To a solution of 6-bromonicotinaldehyde 32-1 (2.0 g, 10.8 mmol) in 1,4-dioxane (20 mL) was added cyclopropylboronic acid (1.5 g, 17.5 mmol), cesium carbonate (10.5 g, 32.2 mmol) and (1,1′-bis(diphenylphosphino)ferrocene)palladium (II) dichloride (200 mg). The mixture was stirred at 90° C. under nitrogen atmosphere overnight. Then the mixture was diluted with water (200 mL), extracted with ethyl acetate (200 mL) twice. The combined organic layers were washed with brine (20 mL), dried over Na₂SO₄₍ₛ₎, filtered and concentrated under vacuum and purified by silica gel column chromatography (petroleum ether:ethyl acetate=10:1) to give the title compound (500 mg, 40% purity from ¹H NMR, 13% yield) as yellow solids. ¹H NMR (400 MHz, CDCl₃) δ 10.02 (s, 1H), 8.86 (d, J=1.6 Hz, 1H), 8.04-8.00 (m, 1H), 7.29 (d, J=8.4 Hz, 1H), 2.16-2.10 (m, 1H), 1.20-1.10 (m, 4H).

Intermediate 32-3

1-(6-Cyclopropylpyridin-3-yl)ethanol

To a solution of 6-cyclopropylnicotinaldehyde 32-2 (500 mg, 40% purity, 1.36 mmol) in tetrahydrofuran (10 mL) was added 1 M methylmagnesium bromide in tetrahydrofuran (3.0 mL, 3.0 mmol) at 0° C. under nitrogen atmosphere. The reaction mixture was stirred at 0° C. for 1 hour. The reaction mixture was quenched with saturated ammonium chloride aqueous solution (15 mL) and the mixture was extracted with dichloromethane (20 mL) for three times. The combined organic layers were washed with brine (20 mL), dried over Na₂SO₄₍ₛ₎, filtered, concentrated and purified by silica gel column chromatography (petroleum ether:ethyl acetate=10:1 to 2:1) to give the title compound (150 mg, 90% purity from ¹H NMR, 61% yield) as yellow oil. ¹H NMR (400 MHz, DMSO-d₆) δ 8.33 (d, J=2.0 Hz, 1H), 7.59-7.56 (m, 1H), 7.21 (d, J=8.0 Hz, 1H), 5.17 (d, J=4.4 Hz, 1H), 4.74-4.68 (m, 1H), 2.08-2.02 (m, 1H), 1.32 (d, J=6.4 Hz, 3H), 0.93-0.85 (m, 4H).

Intermediate 32-4

5-(1-Bromoethyl)-2-cyclopropylpyridine

To a solution of 1-(6-cyclopropylpyridin-3-yl)ethanol 32-3 (5.30 g, 90% purity, 29.2 mmol) in tetrahydrofuran (50 mL) was added triphenylphosphine (15.5 g, 59.1 mmol) and perbromomethane (14.5 g, 43.7 mmol) at 0° C. After being stirred at room temperature for 3 hours, the reaction mixture was concentrated and purified by silica gel column chromatography (petroleum ether:ethyl acetate=20:1 to 8:1) to give the title compound (1.80 g, 70% purity from ¹H NMR, 19% yield) as yellow oil. ¹H NMR (400 MHz, CDCl₃) δ 8.46 (d, J=2.4 Hz, 1H), 7.66 (dd, J=8.0 and 3.2 Hz, 1H), 7.13 (d, J=8.0 Hz, 1H), 5.21-5.15 (m, 1H), 2.4-2.02 (m, 3H), 1.52 (d, J=6.0 Hz, 1H), 1.03-1.00 (m, 4H).

Intermediate 32-5

(3R,7S)-7-(((tert-Butyldiphenylsilyl)oxy)methyl)-9-(1-(6-cyclopropylpyridin-3-yl)ethyl)-2-(3,4-dichlorobenzoyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichloro benzoyl)-3-methyl-1,2,3,4,8,9-hexahydropyrido[4′,3′:3,4]pyrazolo[1,5-a]pyrazin-10(7H)-one Int A (500 mg, 90% purity, 0.695 mmol) and 5-(1-bromoethyl)-2-cyclopropylpyridine 32-4 (360 mg, 70% purity, 1.11 mmol) in 2-methyltetrahydrofuran (10 mL) was added 50% wt. sodium hydroxide aqueous solution (2 mL) and benzyltriethylammonium chloride (110 mg, 0.483 mmol). After being stirred at 20° C. for 4 hours, the mixture was added into water (10 mL) and extracted with dichloromethane (20 mL) twice. The combined organic layers were washed with water (10 mL), brine (10 mL), dried over Na₂SO₄₍ₛ₎ and filtered. The filtrate was concentrated under reduced pressure to get a residue, which was purified by C18 column (acetonitrile:water=20% to 95%) to give the title compound (340 mg, 90% purity from ¹H NMR, 56% yield) as yellow solids. LC-MS (ESI): R_{T}=2.00 min and 2.13 min, mass calcd. for C₄₄H₄₇Cl₂N₅O₃Si 791.3, m/z found 792.4 [M+H]⁺.

Intermediate 32-6

(3R,7S)-9-(1-(6-Cyclopropylpyridin-3-yl)ethyl)-2-(3,4-dichlorobenzoyl)-7-(hydroxymethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-9-(1-(6-cyclopropylpyridin-3-yl)ethyl)-2-(3,4-dichlorobenzoyl)-3-methyl-1,2,3,4,8,9-hexahydropyrido[4′,3′:3,4]pyrazolo[1,5-a]pyrazin-10(7H)-one 32-5 (340 mg, 90% purity, 0.386 mmol) in tetrahydrofuran (3 mL) was added 1 M tetrabutylammonium fluoride in tetrahydrofuran (0.6 mL, 0.6 mmol) at room temperature. After being stirred at room temperature for 1 hour, the reaction mixture was quenched with water (10 mL) and extracted with ethyl acetate (10 mL) twice. The combined organic layers were washed with brine (10 mL) twice, dried over Na₂SO₄₍ₛ₎ and filtered. The filtrate was concentrated and purified by column chromatography on silica gel (DCM:MeOH=20:1) to give desired compound (210 mg, 90% purity from ¹H NMR, 88% yield) as white solids. LC-MS (ESI): R_{T}=1.57 min, mass calcd. for C₂₈H₂₉Cl₂N₅O₃ 553.2, m/z found 554.3 [M+H]⁺.

Intermediate 32-7

(3R,7S)-9-(1-(6-Cyclopropylpyridin-3-yl)ethyl)-2-(3,4-dichlorobenzoyl)-3-methyl-10-oxo-1,2,3,4,7,8,9,10-octahydropyrido[4′,3′:3,4]pyrazolo[1,5-a]pyrazine-7-carboxylic acid

To a solution of (3R,7S)-9-(1-(6-cyclopropylpyridin-3-yl)ethyl)-2-(3,4-dichloro benzoyl)-7-(hydroxymethyl)-3-methyl-1,2,3,4,8,9-hexahydropyrido[4′,3′:3,4]pyrazolo[1,5-a]pyrazin-10(7H)-one 32-6 (210 mg, 90% purity, 0.341 mmol) in acetonitrile (2 mL) were added saturated potassium dihydrogenphosphate aqueous (2 mL), sodium chlorite (85 mg, 80% purity, 0.752 mmol), 2,2,6,6-tetramethyl piperidinooxy (110 mg, 0.704 mmol) and chlorosylsodium (0.5 mL, 10% purity, 0.84 mmol). After being stirred at 0° C. overnight, the reaction mixture was added water (10 mL) and extracted with ethyl acetate (10 mL) for three times. The combined organic layers were washed with brine (15 mL) then dried over Na₂SO₄₍ₛ₎, concentrated and purified by C18 column (acetonitrile:water (0.1% ammonium bicarbonate)=5% to 95%) to give the title product (170 mg, 90% purity from ¹H NMR, 79% yield) as white solids. ¹H NMR (400 MHz, CD₃OD) δ 8.41-8.31 (m, 1H), 7.78-7.58 (m, 3H), 7.43-7.40 (m, 1H), 7.30-7.20 (m, 1H), 5.97-5.36 (m, 2H), 4.70-4.36 (m, 2H), 3.95-3.58 (m, 4H), 3.07-2.97 (m, 1H), 2.78-2.63 (m, 1H), 1.69-1.31 (m, 6H), 1.10-0.97 (m, 4H).

Compound 32

(3R,7S)-9-(1-(6-Cyclopropylpyridin-3-yl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide

A mixture of (3R,7S)-9-(1-(6-cyclopropylpyridin-3-yl)ethyl)-2-(3,4-dichloro benzoyl)-3-methyl-10-oxo-1,2,3,4,7,8,9,10-octahydropyrido[4′,3′:3,4]pyrazolo[1,5-a]pyrazine-7-carboxylic acid 32-7 (235 mg, 90% purity, 0.372 mmol), methylamine hydrochloride (90 mg, 1.33 mmol), 1-hydroxybenzotriazole (175 mg, 1.295 mmol) and 1-(3-dimethylaminopropyl)-3-ethylcarbodiimide hydrochloride (175 mg, 0.913 mmol) in N,N-dimethylformamide (3 mL) at 0° C. was added triethylamine (0.3 mL, 2.15 mmol) dropwise. After being stirred at 0° C. under nitrogen atmosphere for 2 hours, the mixture was acidified to pH=6 with 0.5 M hydrochloride aqueous solution and extracted with ethyl acetate (10 mL) twice. The combined organic layers were washed with water (10 mL) for three times and brine (10 mL), dried over Na₂SO₄₍ₛ₎ and filtered. The filtrate was concentrated under reduced pressure to give a residue, which was purified by C18 column (acetonitrile:water=5% to 95%) to give the title compound (150 mg, 90% purity from ¹H NMR, 52% yield) as white solids. ¹H NMR (400 MHz, CDCl₃) δ 8.43 (d, J=25.6 Hz, 1H), 7.54-7.44 (m, 3H), 7.27-7.24 (m, 1H), 7.12 (d, J=8.0 Hz, 1H), 5.96-5.28 (m, 3H), 5.12-4.22 (m, 3H), 4.11-4.09 (m, 1H), 3.92-3.80 (m, 1H), 3.42-3.38 (m, 1H), 3.11-3.01 (m, 1H), 2.80-2.68 (m, 4H), 2.04-1.98 (m, 1H), 1.57-1.52 (m, 2H), 1.30-1.29 (m, 3H), 0.99-0.97 (m, 4H).

Compounds 32A and 32B

(3R,7S)-9-((R*)-1-(6-Cyclopropylpyridin-3-yl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide (32A), and (3R,7S)-9-((S*)-1-(6-cyclopropylpyridin-3-yl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide (32B)

A racemic mixture of (3R,7S)-9-(1-(6-cyclopropylpyridin-3-yl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide 32 (200 mg, 90% purity, 0.31 mmol) was separated by chiral Prep. HPLC (Column: Chiralpak IB N-5, 5 m 30*250 mm, Mobile Phase: ACN=100% at 20 mL/min, Temp: 30° C., Wavelength: 214 nm), then further purified by Prep. HPLC (Column: sunfire waters C18 (5 μm 19*150 mm), Mobile Phase A: Water (0.1% ammonium bicarbonate), Mobile Phase B: acetonitrile, UV: 214 nm, Flow rate: 15 mL/min, Gradient: 35-85% (% B)) concentrated under reduced pressure to give the title compounds 32A (25.2 mg, 99.5% purity, 14% yield, 100% stereopure) as white solids and 32B (24.9 mg, 99.5% purity, 14% yield, 99.9% stereopure) as white solids.

Compound 32A

LC-MS (ESI): R_{T}=3.696 min, mass calcd. for C₂₉H₃₀Cl₂N₆O₃ 580.2, m/z found 581.3 [M+H]⁺. Chiral analysis (Column: Chiralpak IB N-5, 5 m 4.6*250 mm, Mobile Phase: ACN=100% at 1 mL/min, Temp: 30° C., Wavelength: 254 nm, R_{T}=8.211 min). ¹H NMR (400 MHz, CDCl₃) δ 8.37 (s, 1H), 7.54-7.51 (m, 2H), 7.47 (d, J=7.6 Hz, 1H), 7.27-7.25 (m, 1H), 7.12 (d, J=8.4 Hz, 1H), 6.14-5.83 (m, 2H), 5.64-5.01 (m, 1H), 4.84 (t, J=4.4 Hz, 1H), 4.60-4.33 (m, 1H), 3.90-3.80 (m, 2H), 3.14-2.96 (m, 1H), 2.73-2.68 (m, 4H), 2.07-2.01 (m, 1H), 1.58 (s, 3H), 1.31 (d, J=6.8 Hz, 3H), 1.05-0.97 (m, 4H).

Compound 32B

LC-MS (ESI): R_{T}=3.892 min, mass calcd. for C₂₉H₃₀Cl₂N₆O₃ 580.2, m/z found 581.3 [M+H]⁺. Chiral analysis (Column: Chiralpak IB N-5, 5 m 4.6*250 mm, Mobile Phase: ACN=100% at 1 mL/min, Temp: 30° C., Wavelength: 254 nm, R_{T}=10.881 min). ¹H NMR (400 MHz, CDCl₃) δ 8.43 (s, 1H), 7.54-7.50 (m, 3H), 7.28-7.26 (m, 1H), 7.12 (d, J=8.0 Hz, 1H), 6.08-5.87 (m, 2H), 5.68-5.12 (m, 1H), 4.86-4.80 (m, 1H), 4.57-4.35 (m, 1H), 4.12 (d, J=14.4 Hz, 1H), 3.43 (dd, J=13.6 Hz, 5.2 Hz, 1H), 3.13-2.96 (m, 1H), 2.80 (d, J=5.2 Hz, 3H), 2.73 (d, J=16.4 Hz, 1H), 2.06-1.99 (m, 1H), 1.57 (s, 3H), 1.30 (d, J=6.8 Hz, 3H), 1.00 (d, J=6.8 Hz, 4H).

Compounds 33A and 33B

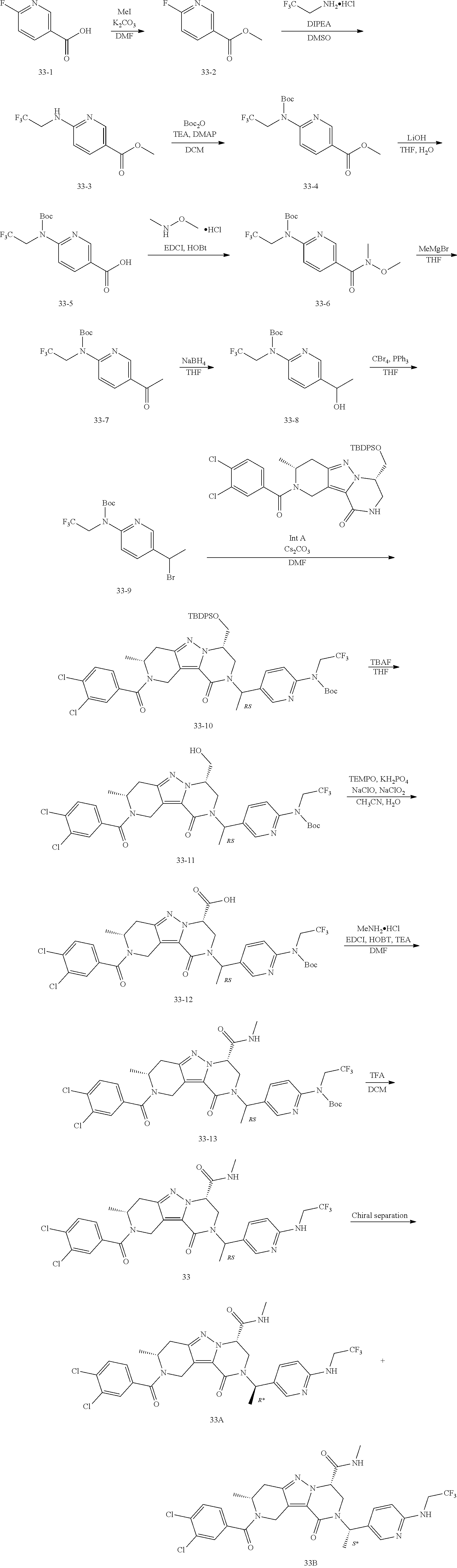

Intermediate 33-2

Methyl 6-fluoronicotinate

To a solution of 6-fluoronicotinic acid 33-1 (3 g, 21.30 mmol) and potassium carbonate (9 g, 65.10 mmol) in N,N-dimethylformamide (50 mL) was added iodomethane (6 g, 42.30 mmol). The mixture was stirred at 20° C. for 3 hours. The reaction mixture was poured into water (100 mL) and extracted with dichloromethane (100 mL) for three times. The organic phase was washed with brine (100 mL) for three times and concentrated in vacuum to give the desired compound (3.3 g, 90% yield, 90% purity from ¹H NMR) as yellow solids. ¹H NMR (300 MHz, CDCl₃) δ 8.89 (s, 1H), 8.44-8.38 (m, 1H), 7.03-6.99 (m, 1H), 3.96 (s, 3H).

Intermediate 33-3

Methyl 6-((2,2,2-trifluoroethyl)amino)nicotinate

To a solution of methyl 6-fluoronicotinate 33-2 (3.3 g, 19.10 mmol, 90% purity) and 2,2,2-trifluoroethanamine hydrochloride (14 g, 103.00 mmol) in dimethyl sulfoxide (100 mL) was added N-ethyl-N-isopropylpropan-2-amine (40 mL, 242 mmol). The mixture was stirred at 120° C. for 12 hours. The reaction mixture was poured into water (150 mL) and extracted with dichloromethane (100 mL) for three times. The organic phase was washed with brine (100 mL) and concentrated in vacuum. The residue was purified by column chromatography on silica gel (petroleum ether:ethyl acetate=10:1) to give desired compound (3.4 g, 75.8% yield, 100% purity from LCMS) as yellow solids. LC-MS (ESI): R_{T}=1.47 min, mass calcd. for C₉H₉F₃N₂O₂234.2, m/z found 235.1 [M+H]⁺.

Intermediate 33-4

Methyl 6-((tert-butoxycarbonyl)(2,2,2-trifluoroethyl)amino)nicotinate

To a solution of methyl 6-((2,2,2-trifluoroethyl)amino)nicotinate 33-3 (3.4 g, 14.50 mmol, 100% purity) and di-tert-butyl dicarbonate (4 g, 18.30 mmol) in dichloromethane (50 mL) was added triethylamine (3 g, 29.60 mmol) and 4-dimethylaminopyridine (180 mg, 1.47 mmol). The mixture was stirred at 20° C. for 2 hours. The reaction mixture was poured into water (100 mL) and extracted with dichloromethane (100 mL) for three times. The organic phase was washed with brine (100 mL) and concentrated in vacuum to give desired compound (4.7 g, 96.8% yield, 100% purity from LCMS) as yellow solids. LC-MS (ESI): R_{T}=1.87 min, mass calcd. for C₁₄H₁₇F₃N₂O₄ 334.3, m/z found 335.2 [M+H]⁺.

Intermediate 33-5

6-((tert-Butoxycarbonyl)(2,2,2-trifluoroethyl)amino)nicotinic acid

To a solution of methyl 6-((tert-butoxycarbonyl)(2,2,2-trifluoroethyl)amino)nicotinate 33-4 (4.7 g, 14.10 mmol, 100% purity) in tetrahydrofuran (50 mL) and water (20 mL) was added lithium hydroxide hydrate (1.2 g, 28.60 mmol). The mixture was stirred at room temperature for 2 hours. The reaction mixture was acidized to pH=4 with 1 M hydrogen chloride and extracted with ethyl acetate (100 mL) for three times. The organic phase was washed with brine (100 mL) and concentrated in vacuum to afford the desired product (4.1 g, 91% yield, 100% purity from LCMS) as yellow solids. LC-MS (ESI): R_{T}=1.24 min, mass calcd. for C₁₃H₁₅F₃N₂O₄ 320.3, m/z found 319.0 [M−H]⁻.

Intermediate 33-6

tert-Butyl (5-(methoxy(methyl)carbamoyl)pyridin-2-yl)(2,2,2-trifluoroethyl)carbamate

To a mixture of 6-((tert-butoxycarbonyl)(2,2,2-trifluoroethyl)amino)nicotinic acid 33-5 (2.0 g, 6.25 mmol, 100% purity), N,O-dimethylhydroxylamine hydrochloride (1.8 g, 18.50 mmol), 1-(3-dimethylaminopropyl)-3-ethylcarbodiimide hydrochloride (2.4 g, 12.50 mmol) and 1H-benzo[d][1,2,3]triazol-1-ol (1.7 g, 12.60 mmol) in dichloromethane (40 mL) was added triethylamine (3.8 g, 37.60 mmol) at room temperature. The mixture was stirred at room temperature for 12 hours. The reaction mixture was added water (100 mL) and extracted with dichloromethane (100 mL) for three times. The organic phase was washed with brine (100 mL) and concentrated in vacuum. The residue was purified by column chromatography on silica gel (petroleum ether:ethyl acetate=7:1) to give desired compound (1.9 g, 83.7% yield, 100% purity from LCMS) as yellow oil. LC-MS (ESI): R_{T}=1.69 min, mass calcd. for C₁₅H₂₀F₃N₃O₄ 363.3, m/z found 364.1 [M+H]⁺.

Intermediate 33-7

tert-Butyl (5-acetylpyridin-2-yl)(2,2,2-trifluoroethyl)carbamate

To a solution of tert-butyl (5-(methoxy(methyl)carbamoyl)pyridin-2-yl)(2,2,2-trifluoroethyl)carbamate 33-6 (1.9 g, 5.23 mmol, 100% purity) in tetrahydrofuran (20 mL) was added 1 M methylmagnesium bromide in tetrahydrofuran (10 mL, 10.00 mmol) at 0° C. The mixture was stirred at 0° C. for 2 hours. The reaction mixture was added ammonium chloride aqueous solution (60 mL) and extracted with dichloromethane (60 mL) for three times. The organic phase was washed with brine (60 mL) and concentrated in vacuum. The residue was purified by column chromatography on silica gel (petroleum ether:ethyl acetate=10:1) to give desired compound (1.4 g, 84% yield, 100% purity from LCMS) as yellow solids. LC-MS (ESI): R_{T}=1.76 min, mass calcd. for C₁₄H₁₇F₃N₂O₃ 318.3, m/z found 319.1 [M+H]⁺.

Intermediate 33-8

tert-Butyl (5-(1-hydroxyethyl)pyridin-2-yl)(2,2,2-trifluoroethyl)carbamate

To a solution of tert-butyl (5-acetylpyridin-2-yl)(2,2,2-trifluoroethyl)carbamate 33-7 (1.4 g, 100% purity, 4.40 mmol) in tetrahydrofuran (15 mL) was added sodium tetrahydroborate (200 mg, 5.29 mmol). The mixture was stirred at room temperature for 2 hours. The mixture was added water (80 mL) and extracted with ethyl acetate (80 mL) for three times. The combined organic layers were washed with brine (80 mL) and concentrated to get the desired compound (1.4 g, 94% yield, 95% purity from ¹H NMR) as yellow oil. ¹H NMR (300 MHz, CDCl₃) δ 8.38-8.35 (m, 1H), 7.74-7.71 (m, 1H), 7.62-7.59 (m, 1H), 5.00-4.92 (m, 1H), 4.76 (q, J=8.7 Hz, 2H), 1.54 (s, 3H), 1.52 (s, 9H).

Intermediate 33-9

tert-Butyl (5-(1-bromoethyl)pyridin-2-yl)(2,2,2-trifluoroethyl)carbamate

To a solution of tert-butyl (5-(1-hydroxyethyl)pyridin-2-yl)(2,2,2-trifluoroethyl)carbamate 33-8 (1.4 g, 95% purity, 4.15 mmol) and perbromomethane (2.1 g, 6.33 mmol) in tetrahydrofuran (20 mL) was added triphenylphosphine (1.64 g, 6.25 mmol) at 0° C. The mixture was stirred at room temperature for 12 hours. The mixture was purified by column chromatography on silica gel (petroleum ether:ethyl acetate=10:1) to give the title compound (1.3 g, 90% purity from ¹H NMR, 73.5% yield) as yellow oil. ¹H NMR (300 MHz, CDCl₃) δ 8.38-8.37 (m, 1H), 7.80-7.76 (m, 1H), 7.70-7.67 (m, 1H), 5.19 (q, J=6.9 Hz, 1H), 4.79 (q, J=8.7 Hz, 2H), 2.05 (d, J=6.9 Hz, 3H), 1.53 (s, 9H).

Intermediate 33-10

tert-Butyl (5-(1-((3R,7S)-7-(((tert-butyldiphenylsilyl)oxy)methyl)-2-(3,4-dichlorobenzoyl)-3-methyl-10-oxo-1,2,3,4,7,8-hexahydropyrido[4′,3′:3,4]pyrazolo[1,5-a]pyrazin-9(10H)-yl)ethyl)pyridin-2-yl)(2,2,2-trifluoroethyl)carbamate

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (1.5 g, 100% purity, 2.32 mmol) and tert-butyl (5-(1-bromoethyl)pyridin-2-yl)(2,2,2-trifluoroethyl)carbamate 33-9 (1.3 g, 90% purity, 3.05 mmol) in N,N-dimethylformamide (30 mL) was added cesium carbonate (2.3 g, 7.06 mmol). The mixture was stirred at 30° C. for 5 hours. The mixture was added water (80 mL) and extracted with ethyl acetate (80 mL) for three times. The combined organic layers were washed with water (80 mL), brine (80 mL) and concentrated. The residue was purified by column chromatography on silica gel (petroleum ether:acetone=10:1) to get the desired compound (2 g, 81.8% yield, 90% purity from ¹H NMR) as white solids. ¹H NMR (300 MHz, DMSO-d₆) δ 8.46-8.31 (m, 1H), 7.88-7.35 (m, 15H), 5.96-5.73 (m, 1H), 5.50-5.18 (m, 1H), 4.85-4.46 (m, 4H), 4.24-3.64 (m, 4H), 3.57-3.37 (m, 1H), 3.00-2.87 (m, 1H), 2.55-2.51 (m, 1H), 1.66-1.51 (m, 3H), 1.46 (s, 9H), 1.20-1.02 (m, 3H), 0.88-0.80 (m, 9H).

Intermediate 33-11

tert-Butyl (5-(1-((3R,7S)-2-(3,4-dichlorobenzoyl)-7-(hydroxymethyl)-3-methyl-10-oxo-1,2,3,4,7,8-hexahydropyrido[4′,3′:3,4]pyrazolo[1,5-a]pyrazin-9(10H)-yl)ethyl)pyridin-2-yl)(2,2,2-trifluoroethyl)carbamate

To a solution of tert-butyl (5-(1-((3R,7S)-7-(((tert-butyldiphenylsilyl)oxy)methyl)-2-(3,4-dichlorobenzoyl)-3-methyl-10-oxo-1,2,3,4,7,8-hexahydropyrido[4′,3′:3,4]pyrazolo[1,5-a]pyrazin-9(10H)-yl)ethyl)pyridin-2-yl)(2,2,2-trifluoroethyl)carbamate 33-10 (2 g, 90% purity, 1.90 mmol) in tetrahydrofuran (20 mL) was added tetrabutylammonium fluoride (5 mL, 5.00 mmol). The mixture was stirred at room temperature for 2 hours. The mixture was added water (50 mL) and extracted with ethyl acetate (50 mL) for three times. The combined organic layers were washed with brine (50 mL) and concentrated. The residue was purified by column chromatography on silica gel (petroleum ether:acetone=5:1) to get the desired compound (1.1 g, 80% yield, 98% purity from LCMS) as white solids. LC-MS (ESI): R_{T}=1.572 min, mass calcd. for C₃₂H₃₅Cl₂F₃N₆O₅ 710.2, m/z found 712.0 [M+H]⁺.

Intermediate 33-12

(3R,7S)-9-(1-(6-((tert-Butoxycarbonyl)(2,2,2-trifluoroethyl)amino)pyridin-3-yl)ethyl)-2-(3,4-dichlorobenzoyl)-3-methyl-10-oxo-1,2,3,4,7,8,9,10-octahydropyrido[4′,3′:3,4]pyrazolo[1,5-a]pyrazine-7-carboxylic acid

To a solution of saturated potassium dihydrogenphosphate aqueous in water (6 mL), sodium chlorite (320 mg, 80% purity, 2.83 mmol) in acetonitrile (10 mL) and 5.5% sodium hypochlorite in water (6 mL) was added 2,2,6,6-tetramethylpiperidinooxy (440 mg, 2.82 mmol) at 0° C. Then tert-butyl (5-(1-((3R,7S)-2-(3,4-dichlorobenzoyl)-7-(hydroxymethyl)-3-methyl-10-oxo-1,2,3,4,7,8-hexahydropyrido[4′,3′:3,4]pyrazolo[1,5-a]pyrazin-9(10H)-yl)ethyl)pyridin-2-yl)(2,2,2-trifluoroethyl)carbamate 33-11 (1.0 g, 98% purity, 1.38 mmol) was added into the solution. After being stirring at room temperature for 5 hours, the reaction mixture was quenched with water (80 mL) and extracted with ethyl acetate (100 mL) twice. The combined organic layers were washed with brine (80 mL) and concentrated. The residue was purified by C18 column (acetonitrile:water=5% to 80%) to give the title compound (930 mg, 94% purity from LCMS, 87% yield) as yellow solids. LC-MS (ESI): R_{T}=1.41 min, mass calcd. for C₃₂H₃₃Cl₂F₃N₆O₆ 724.2, m/z found 725.4 [M+H]⁺.

Intermediate 33-13

tert-Butyl (5-(1-((3R,7S)-2-(3,4-Dichlorobenzoyl)-3-methyl-7-(methylcarbamoyl)-10-oxo-1,2,3,4,7,8-hexahydropyrido[4′,3′:3,4]pyrazolo[1,5-a]pyrazin-9(10H)-yl)ethyl)pyridin-2-yl)(2,2,2-trifluoroethyl)carbamate

To a mixture of (3R,7S)-9-(1-(6-((tert-butoxycarbonyl)(2,2,2-trifluoroethyl)amino)pyridin-3-yl)ethyl)-2-(3,4-dichlorobenzoyl)-3-methyl-10-oxo-1,2,3,4,7,8,9,10-octahydropyrido[4′,3′:3,4]pyrazolo[1,5-a]pyrazine-7-carboxylic acid 33-12 (500 mg, 0.65 mmol, 94% purity), methanamine hydrochloride (137 mg, 2.03 mmol), 1-(3-dimethylaminopropyl)-3-ethylcarbodiimide hydrochloride (253 mg, 1.32 mmol) and 1H-benzo[d][1,2,3]triazol-1-ol (180 mg, 1.33 mmol) in N,N-dimethylformamide (12 mL) was added triethylamine (400 mg, 3.95 mmol) at 0° C. The mixture was stirred at room temperature for 12 hours. The reaction mixture was added water (50 mL) and extracted with ethyl acetate (50 mL) for three times. The organic phase was washed with brine (50 mL) and concentrated in vacuum. The residue was purified by C18 column (acetonitrile:water=5% to 80%) to give the title compound (320 mg, 100% purity from LCMS, 66.8% yield) as white solids. LC-MS (ESI): R_{T}=1.77 min, mass calcd. for C₃₃H₃₆Cl₂F₃N₇O₅ 737.2, m/z found 738.3 [M+H]⁺.

Intermediate 33

(3R,7S)-2-(3,4-Dichlorobenzoyl)-N,3-dimethyl-10-oxo-9-(1-(6-((2,2,2-trifluoroethyl)amino)pyridin-3-yl)ethyl)-1,2,3,4,7,8,9,10-octahydropyrido[4′,3′:3,4]pyrazolo[1,5-a]pyrazine-7-carboxamide

To a solution of tert-butyl (5-(1-((3R,7S)-2-(3,4-dichlorobenzoyl)-3-methyl-7-(methylcarbamoyl)-10-oxo-1,2,3,4,7,8-hexahydropyrido[4′,3′:3,4]pyrazolo[1,5-a]pyrazin-9(10H)-yl)ethyl)pyridin-2-yl)(2,2,2-trifluoroethyl)carbamate 33 (320 mg, 100% purity, 0.43 mmol) in dichloromethane (3 mL) was added 2,2,2-trifluoroacetic acid (1.5 mL). The mixture was stirred at room temperature for 2 hours. The mixture was basified with 2 M sodium bicarbonate aqueous solution to pH=8 and extracted with ethyl acetate (50 mL) for three times. The combined organic layers were washed with brine (50 mL) and concentrated to get desired product (260 mg, 94% yield, 100% purity from LCMS) as white solids. LC-MS (ESI): R_{T}=1.56 min, mass calcd. for C₂₈H₂₈Cl₂F₃N₇O₃ 637.2, m/z found 638.2 [M+H]⁺.

Compounds 33A and 33B

(3R,7S)-2-(3,4-Dichlorobenzoyl)-N,3-dimethyl-10-oxo-9-((R*)-1-(6-((2,2,2-trifluoroethyl)amino)pyridin-3-yl)ethyl)-1,2,3,4,7,8,9,10-octahydropyrido[4′,3′:3,4]pyrazolo[1,5-a]pyrazine-7-carboxamide (33A), and (3R,7S)-2-(3,4-dichlorobenzoyl)-N,3-dimethyl-10-oxo-9-((S*)-1-(6-((2,2,2-trifluoroethyl)amino)pyridin-3-yl)ethyl)-1,2,3,4,7,8,9,10-octahydropyrido[4′,3′:3,4]pyrazolo[1,5-a]pyrazine-7-carboxamide (33B)

A racemate of (3R,7S)-2-(3,4-dichlorobenzoyl)-N,3-dimethyl-10-oxo-9-(1-(6-((2,2,2-trifluoroethyl)amino)pyridin-3-yl)ethyl)-1,2,3,4,7,8,9,10-octahydropyrido[4′,3′:3,4]pyrazolo[1,5-a]pyrazine-7-carboxamide 33 (300 mg, 100% purity, 0.47 mmol) was separated by chiral Prep. HPLC separation condition: (Column: Chiralpak IC m 30*250 mm; Mobile Phase: ACN:IPA=70:30 at 25 mL/min; Temp: 30° C.; Wavelength: 254 nm) to give the title compounds 33A (35 mg, 99.6% purity, 11.6% yield, 99.8% stereopure) and 33B (152 mg, 99.6% purity, 50.4% yield, 99.9% stereopure) as white solids.

Compound 33A

LC-MS (ESI): R_{T}=3.340 min, mass calcd. For C₂₈H₂₈Cl₂F₃N₇O₃ 637.2, m/z found 638.2 [M+H]⁺. Chiral analysis (Column: Chiralpak IC 5 μm 4.6*250 mm; Mobile Phase: ACN:IPA=70:30 at 1 mL/min; Temp: 30° C.; Wavelength: 254 nm, R_{T}=3.548 min). ¹H NMR (400 MHz, DMSO-d₆) δ 7.97-7.87 (m, 1H), 7.82-7.74 (m, 3H), 7.50-7.43 (m, 1H), 7.30-7.14 (m, 2H), 6.62-6.55 (m, 1H), 5.73-5.52 (m, 1H), 5.45-5.17 (m, 1H), 4.98-4.87 (m, 1H), 4.65-4.40 (m, 1H), 4.24-3.87 (m, 4H), 3.43-3.32 (m, 1H), 2.96-2.83 (m, 1H), 2.69-2.52 (m, 1H), 2.40-2.31 (m, 3H), 1.52-1.33 (m, 3H), 1.28-1.10 (m, 3H). ¹⁹F NMR (376 MHz, DMSO-d₆) δ −70.87.

Compound 33B

LC-MS (ESI): R_{T}=3.355 min, mass calcd. For C₂₈H₂₈Cl₂F₃N₇O₃ 637.2, m/z found 638.2 [M+H]⁺. Chiral analysis (Column: Chiralpak IC 5 μm 4.6*250 mm; Mobile Phase: ACN:IPA=70:30 at 1 mL/min; Temp: 30° C.; Wavelength: 254 nm, R_{T}=7.013 min). ¹H NMR (400 MHz, DMSO-d₆) δ 8.06-7.94 (m, 2H), 7.76-7.74 (m, 2H), 7.46-7.31 (m, 2H), 7.22-7.13 (m, 1H), 6.65-6.54 (m, 1H), 5.74-5.54 (m, 1H), 5.46-5.18 (m, 1H), 5.06-4.95 (m, 1H), 4.61-4.43 (m, 1H), 4.24-4.08 (m, 3H), 3.71-3.43 (m, 2H), 2.96-2.84 (m, 1H), 2.69-2.54 (m, 4H), 1.42-1.29 (m, 3H), 1.26-1.10 (m, 3H). ¹⁹F NMR (376 MHz, DMSO-d₆) δ −70.83.

Compounds 34A and 34B

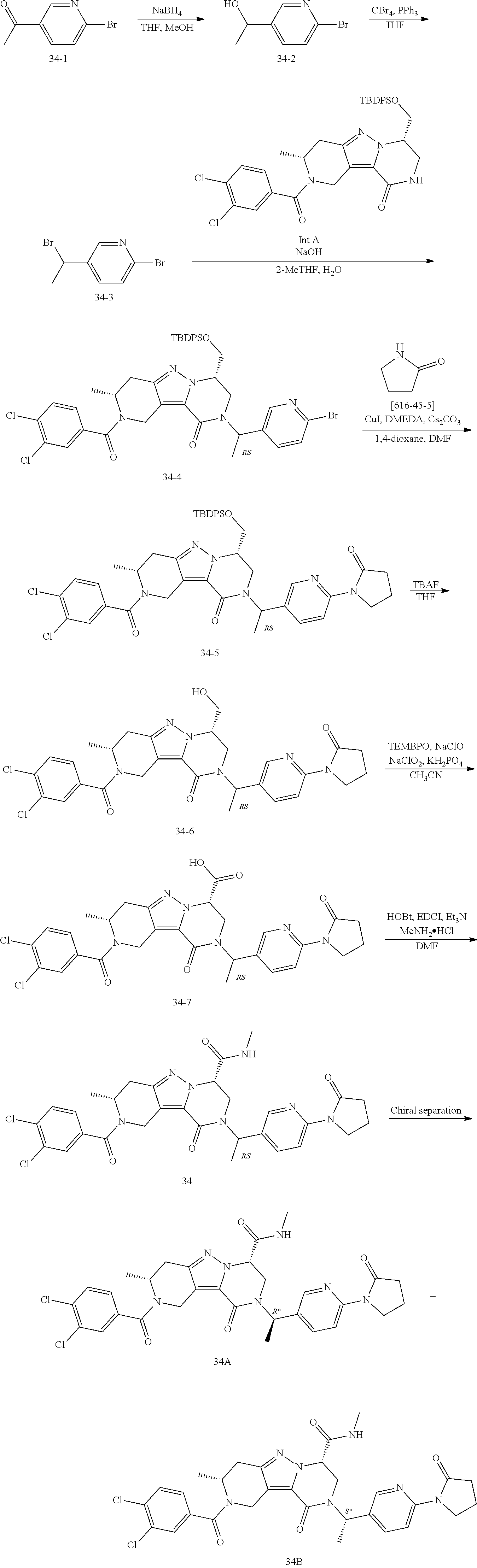

Intermediate 34-2

1-(6-Bromopyridin-3-yl)ethanol

To a solution of 1-(6-bromopyridin-3-yl)ethan-1-one 34-1 (1.0 g, 5.00 mmol) in tetrahydrofuran (10 mL) and methanol (1 mL) was added sodium tetrahydroborate (100 mg, 2.64 mmol). After being stirred at 25° C. for 1 hour, the mixture was quenched with water, and extracted with ethyl acetate (100 mL) for three times. The combined organic layers were washed with brine (100 mL), dried over Na₂SO₄₍ₛ₎, and filtered. The filtrate was concentrated to give the title compound (1.1 g, 87% purity from LCMS, 94.7% yield) as a yellow oil. LC-MS (ESI): R_{T}=1.27 min, mass calcd. for C₇H₈BrNO 202.1, m/z found 203.9 [M+H]⁺.

Intermediate 34-3

2-Bromo-5-(1-bromoethyl)pyridine

To a solution of 1-(6-bromopyridin-3-yl)ethan-1-ol 34-2 (1.1 g, 87% purity, 4.74 mmol) in dichlormethane (10 mL) was added perbromomethane (2.3 g, 6.94 mmol) and triphenylphosphine (1.8 g, 6.86 mmol) at 0° C. After being stirred at room temperature for 2 hours, the mixture was quenched with saturated sodium bicarbonate solution (100 mL), and extracted with dichloroethane (100 mL) for twice. The organic layers were washed with brine (100 mL), dried over Na₂SO₄₍ₛ₎, and filtered. The filtrate was concentrated and purified by silica gel column chromatography (petroleum ether:ethyl acetate=5:1) to give the title compound (1.3 g, 85% purity from LCMS, 88% yield) as a yellow oil. LC-MS (ESI): R_{T}=1.57 min, mass calcd. for C₇H₇Br₂N 264.9, m/z found 265.8 [M+H]⁺.

Intermediate 34-4

(3R,7S)-9-(1-(6-Bromopyridin-3-yl)ethyl)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (1.3 g, 2.01 mmol, 100% purity) and 2-bromo-5-(1-bromoethyl)pyridine 34-3 (700 mg, purity 90%, 2.38 mmol) in 2-methyltetrahydrofuran (6 mL) was added 50% wt. sodium hydroxide in water (6 mL) slowly at 0° C. After being stirred at 0° C. for 2 hours, the mixture was diluted with water (60 mL) and concentrated at room temperature under reduced pressure to remove the volatile. The remained aqueous layer was extracted with ethyl acetate (60 mL) twice and brine (120 mL), dried over Na₂SO₄₍ₛ₎ and filtered. The filtrate was concentrated and purified by C18 column (acetonitrile:water (+0.02% ammonium acetate)=5% to 100%) to give the title compound (1.4 g, 100% purity, 84% yield) as while solids. LC-MS (ESI): R_{T}=2.00 min, mass calcd. for C₄₁H₄₂BrCl₂N₅O₃Si 829.2, m/z found 830.2 [M+H]⁺.

Intermediate 34-5

(3R,7S)-7-(((tert-Butyldiphenylsilyl)oxy)methyl)-2-(3,4-dichlorobenzoyl)-3-methyl-9-(1-(6-(2-oxopyrrolidin-1-yl)pyridin-3-yl)ethyl)-1,2,3,4,8,9-hexahydropyrido[4′,3′:3,4]pyrazolo[1,5-a]pyrazin-10(7H)-one

(3R,7S)-9-(1-(6-bromopyridin-3-yl)ethyl)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one 34-4 (400 mg, 100% purity, 0.481 mmol), pyrrolidin-2-one (400 mg, 4.70 mmol), cesium carbonate (300 mg, 0.921 mmol), N1,N2-dimethylethane-1,2-diamine (30 mg, 0.340 mmol) and copper(I) iodide (24 mg, 0.126 mmol) were mixed in 1,4-dioxane (15 mL) and N,N-dimethylformamide (1.5 mL) at 30° C. After being stirred at 80° C. for 2 hours, the mixture was filtered and concentrated to give the crude. The crude was diluted with water (50 mL), extracted with ethyl acetate (50 mL) twice, brine (120 mL), dried over Na₂SO₄₍ₛ₎ and filtered. The filtrate was concentrated and purified by C18 column (acetonitrile:water (+0.02% ammonium acetate)=5% to 100%) to give the title compound (350 mg, 98% purity, 85% yield) as while solids. LC-MS (ESI): R_{T}=1.79 min and 1.91 min, mass calcd. for C₄₅H₄₈Cl₂N₆O₄Si 834.3, m/z found 835.5 [M+H]⁺.

Intermediate 34-6

(3R,7S)-2-(3,4-Dichlorobenzoyl)-7-(hydroxymethyl)-3-methyl-9-(1-(6-(2-oxopyrrolidin-1-yl)pyridin-3-yl)ethyl)-1,2,3,4,8,9-hexahydropyrido[4′,3′:3,4]pyrazolo[1,5-a]pyrazin-10(7H)-one

To a solution of (3R,7S)-7-(((tert-Butyldiphenylsilyl)oxy)methyl)-2-(3,4-dichlorobenzoyl)-3-methyl-9-(1-(6-(2-oxopyrrolidin-1-yl)pyridin-3-yl)ethyl)-1,2,3,4,8,9-hexahydropyrido[4′,3′:3,4] pyrazolo[1,5-a]pyrazin-10(7H)-one 34-5 (350 mg, 98% purity, 0.410 mmol) in tetrahydrofuran (5 mL) was added 1 M tetrabutylammonium fluoride (0.6 mL, 0.6 mmol) in tetrahydrofuran at 0° C. After being stirred at 0° C. for 4 hours, the mixture was filtered and concentrated under reduced pressure to give crude. The crude was purified by C18 column (acetonitrile:water (+0.02% ammonium acetate)=5% to 100%) to give the title compound (220 mg, 100% purity, 90% yield) as while solids. LC-MS (ESI): R_{T}=1.50 min, mass calcd. for C₂₉H₃₀Cl₂N₆O₄ 596.2, m/z found 597.3 [M+H]⁺.

Intermediate 34-7

(3R,7S)-2-(3,4-Dichlorobenzoyl)-3-methyl-10-oxo-9-(1-(6-(2-oxopyrrolidin-1-yl)pyridin-3-yl)ethyl)-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-7-(hydroxymethyl)-3-methyl-9-(1-(6-(2-oxopyrrolidin-1-yl)pyridin-3-yl)ethyl)-1,2,3,4,8,9-hexahydropyrido[4′,3′:3,4]pyrazolo[1,5-a]pyrazin-10(7H)-one 34-6 (220 mg, 100% purity, 0.368 mmol) in acetonitrile (5 mL) were added 2,2,6,6-tetramethylpiperidinooxy (120 mg, 0.768 mmol), sodium hypochlorite aqueous solution (0.45 mL, 0.756 mmol), sodium chlorite (85 mg, 0.752 mmol), saturated sodium dihydrogenphosphate aqueous solution (5 mL) at 0° C. After being stirred at 25° C. for 16 hours, the mixture was diluted with water (50 mL) and extracted with ethyl acetate (50 mL) twice. The combined organic layers were washed with brine (50 mL), dried over Na₂SO₄₍ₛ₎ and filtered. The filtrate was concentrated to give the crude (200 mg, 82% purity, 73% yield). LC-MS (ESI): R_{T}=1.93 min, mass calcd. for C₂₉H₃₀Cl₂N₆O₅ 610.2, m/z found 611.3 [M+H]⁺.

Compound 34

(3R,7S)-2-(3,4-Dichlorobenzoyl)-N,3-dimethyl-10-oxo-9-(1-(6-(2-oxopyrrolidin-1-yl)pyridin-3-yl)ethyl)-1,2,3,4,7,8,9,10-octahydropyrido[4′,3′:3,4]pyrazolo[1,5-a]pyrazine-7-carboxamide

To a solution of (3R,7S)-2-(3,4-dichlorobenzoyl)-3-methyl-10-oxo-9-(1-(6-(2-oxopyrrolidin-1-yl)pyridin-3-yl)ethyl)-1,2,3,4,7,8,9,10-octahydropyrido[4′,3′:3,4]pyrazolo[1,5-a]pyrazine-7-carboxylic acid 34-7 (200 mg, 0.268 mmol, purity 82%) in N,N-dimethylformamide (6 mL) were added methanamine hydrochloride (50 mg, 0.704 mmol), 1H-benzo[d][1,2,3]triazol-1-ol (75 mg, 0.56 mmol), 1-(3-dimethylaminopropyl)-3-ethylcarbodiimide hydrochloride (100 mg, 0.522 mmol) and triethylamine (0.3 mL, 2.08 mmol) at 0° C. After being stirred at 0° C. for 2 hours, the mixture was added ethyl acetate (50 mL) washed with water (50 mL) twice, brine (100 mL), dried over Na₂SO₄₍ₛ₎ and filtered. The filtrate was concentrated under reduced pressure and purified by C18 column (acetonitrile:water (+0.02% ammonium acetate)=5% to 100%) to give the title compound (120 mg, 100% purity, 72% yield) as while solids. LC-MS (ESI): R_{T}=1.48 min, mass calcd. for C₃₀H₃₁Cl₂N₇O₄ 623.2, m/z found 624.5 [M+H]⁺.

Compounds 34A and 34B

(3R,7S)-2-(3,4-Dichlorobenzoyl)-N,3-dimethyl-10-oxo-9-((R*)-1-(6-(2-oxopyrrolidin-1-yl)pyridin-3-yl)ethyl)-1,2,3,4,7,8,9,10-octahydropyrido[4′,3′:3,4]pyrazolo[1,5-a]pyrazine-7-carboxamide (34A), and (3R,7S)-2-(3,4-dichlorobenzoyl)-N,3-dimethyl-O-oxo-9-((S*)-1-(6-(2-oxopyrrolidin-1-yl)pyridin-3-yl)ethyl)-1,2,3,4,7,8,9,10-octahydropyrido[4′,3′:3,4]pyrazolo[1,5-a]pyrazine-7-carboxamide (34B)

A racemic mixture of (3R,7S)-2-(3,4-dichlorobenzoyl)-N,3-dimethyl-10-oxo-9-(1-(6-(2-oxopyrrolidin-1-yl)pyridin-3-yl)ethyl)-1,2,3,4,7,8,9,10-octahydropyrido[4′,3′:3,4]pyrazolo[1,5-a]pyrazine-7-carboxamide 34 (120 mg, 0.192 mmol, 100% purity) was separated by chiral HPLC (separation condition: Column: Chiralpak IE, 5 m 30*250 mm, MeOH:DCM=70:30, 25 ml/min; Col. Temp: 30° C.; Wavelength: 254 nm) to afford the desired products compound 34A (40 mg, 98.6% purity, 33% yield, 100% stereopure) and compound 34B (40 mg, 98.8% purity, 33% yield, 100% stereopure) as white solids.

Compound 34A

LC-MS (ESI): R_{T}=2.945 min, mass calcd. for C₃₀H₃₁Cl₂N₇O₄ 623.2, m/z found 624.5 [M+H]⁺. Chiral analysis (Column: Chiralpak IE 5 μm 4.6*250 mm; Mobile Phase: MeOH:DCM=70:30 at 1 mL/min; Temp: 30° C.; Wavelength: 254 nm, R_{T}=7.513 min). ¹H NMR δ 8.38 (d, J=8.8 Hz, 1H), 8.29 (s, 1H), 7.59-7.51 (m, 3H), 7.28-7.26 (m, 1H), 5.91-5.43 (m, 3H), 4.86-4.39 (m, 3H), 4.15-4.05 (m, 2H), 3.90-3.82 (m, 2H), 3.05 (br s, 1H), 2.73-2.64 (m, 6H), 2.17-2.09 (m, 2H), 1.61 (d, J=6.8 Hz, 3H), 1.31 (d, J=6.8 Hz, 3H).

Compound 34B

LC-MS (ESI): R_{T}=3.065 min, mass calcd. for C₃₀H₃₁Cl₂N₇O₄ 623.2, m/z found 624.5 [M+H]⁺. Chiral analysis (Column: Chiralpak IE 5 μm 4.6*250 mm; Mobile Phase: MeOH:DCM=70:30 at 1 mL/min; Temp: 30° C.; Wavelength: 254 nm, R_{T}=11.058 min). ¹H NMR δ 8.39-8.36 (m, 2H), 7.67-7.65 (m, 1H), 7.54-7.52 (m, 2H), 7.28-7.26 (m, 1H), 5.99-5.36 (m, 3H), 4.83-4.35 (m, 3H), 4.14-4.02 (m, 3H), 3.43-3.38 (m, 1H), 3.14-2.96 (m, 1H), 2.80-2.73 (m, 3H), 2.68-2.64 (m, 3H), 2.17-2.10 (m, 2H), 1.60 (d, J=6.8 Hz, 3H), 1.30-1.29 (m, 3H).

Compounds 35A and 35B

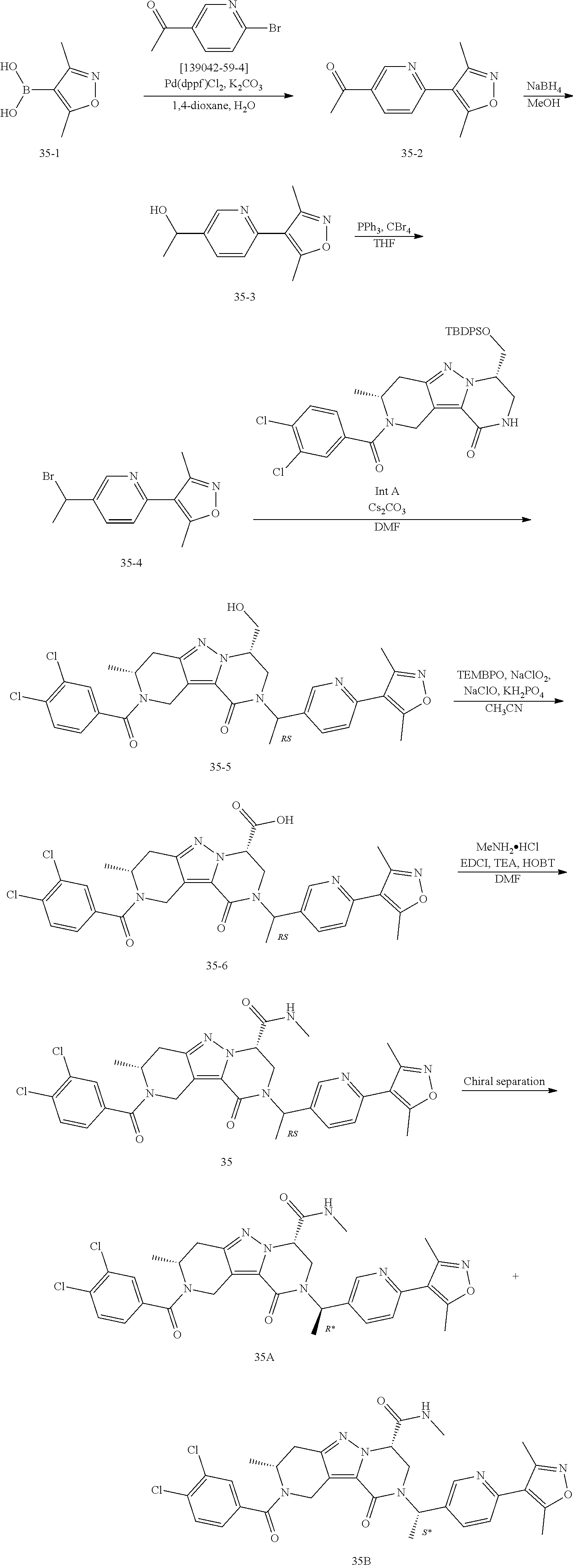

Intermediate 35-2

1-(6-(3,5-Dimethylisoxazol-4-yl)pyridin-3-yl)ethanone

A solution of 5-Acetyl-2-bromopyridine (5.00 g, 25.0 mmol), (3,5-dimethylisoxazol-4-yl)boronic acid 35-1 (4.00 g, 28.4 mmol), 1,1′-bis(diphenylphosphino)ferrocene-palladium(II)dichloride dichloromethane complex (2.00 g, 2.45 mmol) and potassium carbonate (10.0 g, 72.4 mmol) in 1,4-dioxane (25 mL) and water (5 mL) was heated at 90° C. overnight under nitrogen atmosphere. The resulting mixture was cooled down and concentrated, the residue was purified by silica gel chromatography column (ethyl acetate:petroleum ether=1:5) to obtain the title compound (4.00 g, 90% purity from ¹H NMR, 67% yield) as yellow solids. LC-MS (ESI): R_{T}=1.37 min, mass calcd. for C₁₂H₁₂N₂O₂ 216.1, m/z found 217.1 [M+H]⁺. ¹H NMR (300 MHz, CDCl₃) δ 9.23 (s, 1H), 8.36-8.32 (m, 1H), 7.50 (d, J=8.4 Hz, 1H), 2.72 (s, 3H), 2.69 (s, 3H), 2.53 (s, 3H).

Intermediate 35-3

1-(6-(3,5-Dimethylisoxazol-4-yl)pyridin-3-yl)ethanol

To the solution of 1-(6-(3,5-dimethylisoxazol-4-yl)pyridin-3-yl)ethenone 35-2 (4.00 g, 90% purity, 0.807 mmol) in methanol (4 mL) was added sodium borohydride (100 mg, 2.64 mmol) at 0° C. After being stirred at 30° C. for 3 hours, the mixture was quenched with saturated ammonium chloride aqueous solution (50 mL) at 0° C., and extracted with ethyl acetate (100 mL) for twice. The combined organic layers were washed by brine (50 mL), dried over Na₂SO₄₍ₛ₎, filtered and concentrated to give the title compound (3.80 g, 79% purity from LCMS, 82% yield) as yellow oil. LC-MS (ESI): R_{T}=1.26 min, mass calcd. for C₁₂H₁₄N₂O₂ 218.1, m/z found 219.1 [M+H]⁺. ¹H NMR (300 MHz, CDCl₃) δ 8.70 (s, 1H), 7.84 (d, J=7.8 Hz, 1H), 7.35 (d, J=8.1 Hz, 1H), 5.12-4.97 (m, 1H), 2.59 (s, 3H), 2.45 (s, 3H), 1.87-1.79 (m, 1H), 1.60 (d, J=6.3 Hz, 3H).

Intermediate 35-4

4-(5-(1-Bromoethyl)pyridin-2-yl)-3,5-dimethylisoxazole

To a solution of 1-(6-(3,5-dimethylisoxazol-4-yl)-3-yl)ethanol 35-3 (3.60 g, 79% purity, 12.9 mmol) in tetrahydrofuran (36 mL) was added triphenylphosphine (5.50 g, 21.0 mmol) and perbromomethane (5.50 g, 16.6 mmol) at 0° C. After being stirred at 25° C. for 2 hours, the reaction was concentrated to get a residue, which was purified by column chromatography on silica gel (petroleum ether:ethyl acetate=20:1 to 5:1) to give the desired compound (2.80 g, 90% purity from ¹H NMR, 69% yield) as yellow oil. LC-MS (ESI): R_{T}=1.59 min, mass calcd. for C₁₂H₁₃BrN₂O 280.0, m/z found 281.0 [M+H]⁺. ¹H NMR (300 MHz, CDCl₃) δ 8.74 (s, 1H), 7.88 (d, J=6.6 Hz, 1H), 7.36 (d, J=8.1 Hz, 1H), 5.31-5.24 (m, 1H), 2.61 (s, 3H), 2.47 (s, 3H), 2.13 (d, J=6.9 Hz, 3H).

Intermediate 35-5

(3R,7S)-2-(3,4-Dichlorobenzoyl)-9-(1-(6-(3,5-dimethylisoxazol-4-yl)pyridin-3-yl)ethyl)-7-(hydroxymethyl)-3-methyl-1,2,3,4,8,9-hexahydropyrido[4′,3′:3,4]pyrazolo[1,5-a]pyrazin-10(7H)-one

To a mixture of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (2.00 g, 90% purity, 2.78 mmol) and 4-(5-(1-bromoethyl)pyridin-2-yl)-3,5-dimethylisoxazole 35-4 (2.00 g, 90% purity, 6.40 mmol) in N,N-dimethylformamide (30 mL) was added cesium carbonate (4.50 g, 13.8 mmol). After being heated at 70° C. for 3 hours, the mixture was diluted with water (50 mL) and extracted with ethyl acetate (50 mL) twice. The combined organic layers were washed with brine (50 mL), dried over Na₂SO₄₍ₛ₎ and filtered. The filtrate was concentrated and purified by column chromatography on silica gel (petroleum ether: ethyl acetate=0:1) to give the desired compound (1.20 g, 60% purity from LCMS, 43% yield) as yellow solids. LC-MS (ESI): R_{T}=0.98 min and 1.00 min, mass calcd. for C₃₀H₃₀Cl₂N₆O₄ 608.2, m/z found 609.1 [M+H]⁺.

Intermediate 35-6

(3R,7S)-2-(3,4-Dichlorobenzoyl)-9-(1-(6-(3,5-dimethylisoxazol-4-yl)pyridin-3-yl)ethyl)-3-methyl-10-oxo-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-9-(1-(6-(3,5-dimethylisoxazol-4-yl)pyridin-3-yl)ethyl)-7-(hydroxymethyl)-3-methyl-1,2,3,4,8,9-hexahydropyrido[4′,3′:3,4]pyrazolo[1,5-a]pyrazin-10(7H)-one 35-5 (1.00 g, 60% purity, 0.984 mmol) in acetonitrile (13 mL) was added saturated potassium dihydrogenphosphate aqueous solution (13 mL), sodium chlorite (300 mg, 80% purity, 2.65 mmol), 2,2,6,6-tetramethylpiperidinooxy (500 mg, 3.20 mmol) and sodium hypochlorite aqueous solution (2 mL, 10% purity, 3.36 mmol) at 0° C. After being stirred at 20° C. for 4 hours, the reaction mixture was quenched with saturated sodium thiosulfate aqueous solution (10 mL), acidized with 1 M hydrochloric acid solution to pH=4˜5, extracted with ethyl acetate (30 mL) for three times. The combined organic layers were washed with brine (20 mL), dried over Na₂SO₄₍ₛ₎, and filtered. The filtrate was concentrated and purified by C18 column (acetonitrile:water=5% to 95%) to give the title compound (500 mg, 95% purity from LCMS, 77% yield) as white solids. LC-MS (ESI): R_{T}=1.31 min &1.34 min, mass calcd. for C₃₀H₂₈Cl₂N₆O₅ 622.1, m/z found 623.1 [M+H]⁺.

Compound 35

(3R,7S)-2-(3,4-Dichlorobenzoyl)-9-(1-(6-(3,5-dimethylisoxazol-4-yl)pyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide

To a solution of (3R,7S)-2-(3,4-dichlorobenzoyl)-9-(1-(6-(3,5-dimethylisoxazol-4-yl)pyridin-3-yl)ethyl)-3-methyl-10-oxo-1,2,3,4,7,8,9,10-octahydropyrido[4′,3′:3,4]pyrazolo[1,5-a]pyrazine-7-carboxylic acid 35-6 (400 mg, 95% purity, 0.609 mmol), 1H-benzo[d][1,2,3]triazol-1-ol (150 mg, 1.11 mmol), 1-(3-dimethylaminopropyl)-3-ethylcarbodiimide hydrochloride (220 mg, 1.15 mmol) and methanamine hydrochloride (120 mg, 1.78 mmol) in N,N-dimethylformamide (8 mL) was added triethylamine (150 mg, 1.11 mol) at 0° C. After being stirred at room temperature for 2 hours, the mixture was diluted with water (50 mL) and extracted with ethyl acetate (50 mL) twice. The combined organic layers were washed with brine (10 mL), dried over Na₂SO₄₍ₛ₎ and filtered. The filtrate was concentrated to give the title compound (400 mg, 90% purity from LCMS, 93% yield) as white solids. LC-MS (ESI): R_{T}=1.53 min and 1.56 min, mass calcd. for C₃₁H₃₁Cl₂N₇O₄ 635.2, m/z found 636.1 [M+H]⁺.

Compounds 35A and 35B

(3R,7S)-2-(3,4-Dichlorobenzoyl)-9-((R*)-1-(6-(3,5-dimethylisoxazol-4-yl)pyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide (35A), and (3R,7S)-2-(3,4-dichlorobenzoyl)-9-((S*)-1-(6-(3,5-dimethylisoxazol-4-yl)pyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide (35B)

A racemic mixture of (3R,7S)-2-(3,4-dichlorobenzoyl)-9-(1-(6-(3,5-dimethylisoxazol-4-yl)pyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide 35 (400 mg, 90% purity, 0.566 mmol) was separated by Prep-HPLC (acetonitrile:water (0.1% ammonium bicarbonate)=30%-50%) to afford the title compound 35A (39 mg, 97.4% purity from Chiral HPLC, 10.6% yield, 97.4% stereopure) as white solids and compound 35B (79 mg, 98.6% purity from Chiral HPLC, 21.6% yield, 98.6% stereopure) as white solids.

Compound 35A

LC-MS (ESI): R_{T}=3.559 min, mass calcd. for C₃₁H₃₁Cl₂N₇O₄ 635.2, m/z found 636.2 [M+H]⁺. Chiral analysis (Column: Chiralpak IE 5 μm, 4.6*250 mm; Mobile Phase: ACN:IPA=70:30 at 1 mL/min; Temp 30° C.; Wavelength: 254 nm; R_{T}=7.922 min). ¹H NMR (400 MHz, CDCl₃) δ 8.61 (s, 1H), 7.69 (d, J=9.6 Hz, 1H), 7.54-7.52 (m, 2H), 7.32-7.26 (m, 2H), 5.98-5.15 (m, 3H), 4.89-4.87 (m, 1H), 4.63-4.30 (m, 2H), 3.99-3.89 (m, 2H), 3.20-2.95 (m, 1H), 2.70-2.68 (m, 1H), 2.68 (d, J=5.2 Hz, 3H), 2.58 (s, 3H), 2.43 (s, 3H), 1.66 (d, J=6.8 Hz, 3H), 1.31 (d, J=6.4 Hz, 3H).

Compound 35B

LC-MS (ESI): R_{T}=3.596 min, mass calcd. for C₃₁H₃₁Cl₂N₇O₄ 635.2, m/z found 636.3 [M+H]⁺. Chiral analysis (Column: Chiralpak IE 5 μm, 4.6*250 mm; Mobile Phase: ACN:IPA=70:30 at 1 mL/min; Temp 30° C.; Wavelength: 254 nm; R_{T}=10.583 min). ¹H NMR (400 MHz, CDCl₃) δ 8.69 (s, 1H), 7.74 (d, J=8.0 Hz, 1H), 7.54-7.52 (m, 2H), 7.33 (d, J=8.0 Hz, 1H), 7.28-7.26 (m, 1H), 6.03-5.20 (m, 3H), 4.88 (s, 1H), 4.65-4.33 (m, 2H), 4.19 (d, J=13.2 Hz, 1H), 3.53-3.49 (m, 1H), 3.16-2.98 (m, 1H), 2.81 (d, J=4.8 Hz, 3H), 2.74-2.68 (m, 1H), 2.58 (s, 3H), 2.43 (s, 3H), 1.66 (d, J=7.6 Hz, 3H), 1.30 (d, J=6.8 Hz, 3H).

Compounds 36A and 36B

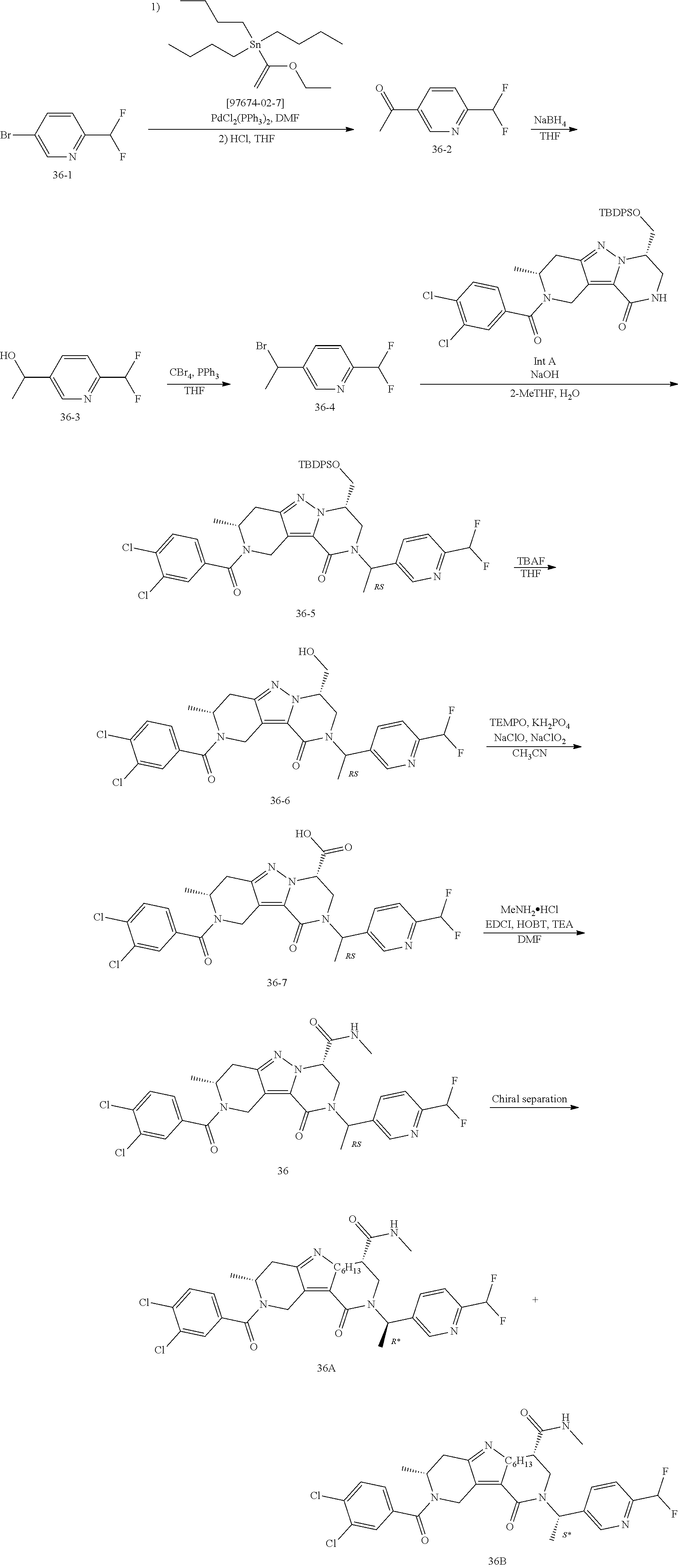

Intermediate 36-2

1-(6-(Difluoromethyl)pyridin-3-yl)ethanone

To a solution of degassed solution of 5-bromo-2-(difluoromethyl)pyridine 36-1 (500 mg, 2.40 mmol) and tributyl(1-ethoxyethenyl)stannane (1.0 mL, 2.96 mmol) in N,N-dimethylformamide (10 mL) was added bis(triphenylphosphine)palladium(II) chloride (20 mg, 0.03 mmol). After being stirred at 100° C. for 2.5 hours, the reaction mixture was diluted with ether (20 mL) and treated with potassium fluoride aqueous solution (700 mg of potassium fluoride in 20 mL water). After being stirred vigorously for 1 hour, the mixture was filtered with kieselguhr. The filtrate was diluted with ethyl acetate (30 mL), washed with saturated sodium bicarbonate aqueous solution (30 mL), brine (30 mL), dried over Na₂SO₄₍ₛ₎, filtered and concentrated under reduced pressure to give the crude. To a solution of the crude in tetrahydrofuran (10 mL) was added 2M hydrochloride aqueous solution (10 mL, 20 mmol) at room temperature. After being stirred at room temperature for 15 minutes, the reaction mixture was quenched with water (20 mL), extracted with ethyl acetate (30 mL) twice. The combined organic layers were washed with brine (30 mL), dried over Na₂SO₄₍ₛ₎ and filtered. The filtrate was concentrated under reduced pressure to give the residue and purified by silica gel column chromatography (petroleum ether:ethyl acetate=4:1) to give the title compound (400 mg, 90% purity by ¹H NMR, 87.5% yield) as yellow oil. LC-MS (ESI): R_{T}=1.30 min, mass calcd. for C₈H₇F₂NO 171.1, m/z found 172.1 [M+H]⁺. ¹H NMR (400 MHz, CDCl₃) δ 9.18 (d, J=1.2 Hz, 1H), 8.38 (dd, J=8.0 Hz, 1.6 Hz, 1H), 7.77 (d, J=8.0 Hz, 1H), 6.69 (t, J=55.2 Hz, 1H), 2.68 (s, 3H).

Intermediate 36-3

1-(6-(Difluoromethyl)pyridin-3-yl)ethanol

To a solution of 1-(6-(difluoromethyl)-3-yl) 36-2 (400 mg, 90% purity, 2.10 mmol) in tetrahydrofuran (5 mL) was added sodium borohydride (160 mg, 4.23 mmol) at 0° C. After addition, the mixture was stirred at room temperature for 2 hours. The mixture was quenched with water (30 mL). The organic phase was separated, and the aqueous phase was extracted with ethyl acetate (30 mL) for three times. The combined organic layers were washed with brine (30 mL), dried over Na₂SO₄₍ₛ₎, filtered and concentrated under reduced pressure to get the crude, which was purified by C18 column (acetonitrile:water=30% to 55%) to give the title compound (264 mg, 95% purity from ¹H NMR, 68.9% yield) as yellow oil. LC-MS (ESI): R_{T}=1.14 min, mass calcd. for C₈H₉F₂NO 173.1, m/z found 174.0 [M+H]⁺. ¹H NMR (400 MHz, CDCl₃) δ 8.64 (s, 1H), 7.88 (dd, J=8.0, 2.0 Hz, 1H), 7.63 (d, J=8.0 Hz, 1H), 6.64 (t, J=55.6 Hz, 1H), 5.03-5.02 (m, 1H), 2.03 (d, J=3.2 Hz, 1H), 1.55 (d, J=6.4 Hz, 3H).

Intermediate 36-4

5-(1-Bromoethyl)-2-(difluoromethyl)pyridine

To a solution of 1-(6-(difluoromethyl)-3-yl)ethanol 36-3 (500 mg, 95% purity, 2.74 mmol) in tetrahydrofuran (15 mL) were added triphenylphosphine (1.2 g, 4.58 mmol) and perbromomethane (1.2 g, 3.62 mmol) at 0° C. After being stirred at 25° C. for 1 hour, the mixture was filtered. The filtrate was concentrated under reduced pressure to give the residue, which was purified by silica gel column chromatography (petroleum ether:ethyl acetate=10:1) to give the title compound (520 mg, 95% purity by ¹H NMR, 76.3% yield) as yellow oil. LC-MS (ESI): R_{T}=1.60 min, mass calcd. for C₈H₈BrF₂N 235.0, m/z found 236.0 [M+H]⁺. ¹H NMR (400 MHz, CDCl₃) δ 8.69 (d, J=1.2 Hz, 1H), 7.94 (dd, J=8.0 and 2.0 Hz, 1H), 7.64 (d, J=8.0 Hz, 1H), 6.64 (t, J=55.2 Hz, 1H), 5.20 (q, J=7.2 Hz, 1H), 2.07 (d, J=7.2 Hz, 3H).

Intermediate 36-5

(3R,7S)-7-(((tert-Butyldiphenylsilyl)oxy)methyl)-2-(3,4-dichlorobenzoyl)-9-(1-(6-(difluoromethyl)pyridin-3-yl)ethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (250 mg, 100% purity, 0.39 mmol) and 5-(1-bromoethyl)-2-(difluoromethyl)pyridine 36-4 (120 mg, 95% purity, 0.48 mmol) in 2-methyltetrahydrofuran (3 mL) was added 50% wt. sodium hydroxide in water (3 mL) slowly at 30° C. After being stirred at 30° C. for 2 hours, the reaction mixture was diluted with water (30 mL) and extracted with ethyl acetate (30 mL) twice. The combined organic layers were washed with brine (30 mL) twice, dried over Na₂SO₄₍ₛ₎ and filtered. The filtrate was concentrated under reduced pressure to give crude compound (400 mg, 72.5% purity from LCMS, 93.6% yield) as yellow solids. The crude was used directly. LC-MS (ESI): R_{T}=2.30 min and 2.32 min, mass calcd. for C₄₂H₄₃Cl₂F₂N₅O₃Si 801.3, m/z found 802.3 [M+H]⁺.

Intermediate 36-6

(3R,7S)-2-(3,4-Dichlorobenzoyl)-9-(1-(6-(difluoromethyl)pyridin-3-yl)ethyl)-7-(hydroxymethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-9-(1-(6-(difluoromethyl)pyridin-3-yl)ethyl)-3-methyl-1,2,3,4,8,9-hexahydropyrido[4′,3′:3,4]pyrazolo[1,5-a]pyrazin-10(7H)-one 36-5 (400 mg, 72.5% purity, 0.36 mmol) in tetrahydrofuran (5 mL) was added 1M tetrabutylammonium fluoride in tetrahydrofuran (1 mL, 1 mmol) at 0° C. After being stirred at 0° C. for 1 hour, the mixture was concentrated under reduced pressure to give crude. The crude was purified by C18 column (acetonitrile:water=30% to 70%) to give the title compound (210 mg, 96.4% purity from LCMS, 99.3% yield) as yellow oil. LC-MS (ESI): R_{T}=1.54 min, mass calcd. for C₂₆H₂₅Cl₂F₂N₅O₃ 563.1, m/z found 564.2 [M+H]⁺.

Intermediate 36-7

(3R,7S)-2-(3,4-Dichlorobenzoyl)-9-(1-(6-(difluoromethyl)pyridin-3-yl)ethyl)-3-methyl-10-oxo-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-9-(1-(6-(difluoromethyl)pyridin-3-yl)ethyl)-7-(hydroxymethyl)-3-methyl-1,2,3,4,8,9-hexahydropyrido[4′,3′:3,4]pyrazolo[1,5-a]pyrazin-10(7H)-one 36-6 (210 mg, 96.4% purity, 0.36 mmol) in acetonitrile (3 mL) was added 2,2,6,6-tetramethylpiperidinooxy (115 mg, 0.74 mmol), sodium chlorite (80 mg, 80% purity, 0.71 mmol), saturated potassium dihydrogen phosphate aqueous solution (3 mL) and sodium hypochlorite aqueous solution (0.8 mL, 5.5% purity, 0.74 mmol), at 0° C. After being stirred at 20° C. overnight, the mixture was diluted with saturated sodium sulfite aqueous solution (20 mL) and extracted with ethyl acetate (30 mL) twice. The combined organic layers were washed with brine (30 mL), dried over Na₂SO₄₍ₛ₎ and filtered. The filtrate was concentrated to give the crude, which was purified by C18 (acetonitrile:water=35% to 55%) to give the desired product (200 mg, 100% purity from LCMS, 96.4% yield) as white solids. LC-MS (ESI): R_{T}=1.29 min, mass calcd. for C₂₆H₂₃Cl₂F₂N₅O₄ 577.1, m/z found 578.1 [M+H]⁺.

Compound 36

(3R,7S)-2-(3,4-Dichlorobenzoyl)-9-(1-(6-(difluoromethyl)pyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide

A mixture of (3R,7S)-2-(3,4-dichlorobenzoyl)-9-(1-(6-(difluoromethyl)pyridin-3-yl)ethyl)-3-methyl-10-oxo-1,2,3,4,7,8,9,10-octahydropyrido[4′,3′:3,4]pyrazolo[1,5-a]pyrazine-7-carboxylic acid 36-7 (500 mg, 84% purity, 0.726 mmol), methylamine hydrochloride (150 mg, 2.22 mmol), 1-(3-dimethylaminopropyl)-3-ethylcarbodiimide hydrochloride (280 mg, 1.46 mmol) and 1H-benzo[d][1,2,3]triazol-1-ol (200 mg, 1.48 mmol) in N,N-dimethylformamide (15 mL) at 0° C. was added trimethylamine (0.6 mL, 4.32 mmol). After being stirred at room temperature under nitrogen overnight, the mixture was acidified to pH=6 with 0.5M hydrochloride aqueous solution and extracted with ethyl acetate (20 mL) twice. The combined organic layers were concentrated under reduced pressure to give a residue, which was purified by C18 column (acetonitrile:water=05% to 80%) to give the title compound (360 mg, 100% purity, 84% yield) as white solids. LC-MS (ESI): R_{T}=1.61 min, mass calcd. for C₂₇H₂₆Cl₂F₂N₆O₃ 590.1, m/z found 591.3 [M+H]⁺.

Compounds 36A and 36B

(3R,7S)-2-(3,4-Dichlorobenzoyl)-9-((R*)-1-(6-(difluoromethyl)pyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide (36A), and (3R,7S)-2-(3,4-dichlorobenzoyl)-9-((S*)-1-(6-(difluoromethyl)pyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide (36B)

The racemic mixture of (3R,7S)-2-(3,4-dichlorobenzoyl)-9-(1-(6-(difluoromethyl)pyridin-3-yl)ethyl)-N,3-dimethyl-10-oxo-1,2,3,4,7,8,9,10-octahydropyrido[4′,3′:3,4]pyrazolo[1,5-a]pyrazine-7-carboxamide 36 (440 mg, 100% purity, 0.744 mmol) was separated by chiral Prep. HPLC (separation method: Column: Chiralpak IB N-5, 5 m 30*250 mm; Mobile Phase: 100% ACN at 60 mL/min; Col. Temp: 30° C.; Wavelength: 254 nm, Back pressure: 100 bar) to give the title compound 36A (101.3 mg, 97.6% purity, 22% yield, 100% stereopure) as white solids and compound 36B (178.1 mg, 99.5% purity, 40% yield, 99.9% stereopure) as white solids.

Compound 36A

LC-MS (ESI): R_{T}=3.024 min, mass calcd. for C₂₇H₂₆Cl₂F₂N₆O₃ 590.1, m/z found 591.2 [M+H]⁺. Chiral analysis (Column: Superchiral IB N-5, 5 μm 4.6*250 mm; Mobile Phase: 100% CH₃CN at 1.0 mL/min; Temp: 30° C.; Wavelength: 254 nm, Back pressure: 100 bar; R_{T}=4.974 min). ¹H NMR (400 MHz, CDCl₃) δ 8.59 (s, 1H), 7.79 (d, J=8.0 Hz, 1H), 7.62 (d, J=8.0 Hz, 1H), 7.54-7.51 (m, 2H), 7.28-7.25 (m, 1H), 6.64 (t, J=55.6 Hz, 1H), 6.07-5.29 (m, 3H), 4.89-4.29 (m, 3H), 4.00-3.89 (m, 2H), 3.17-2.98 (m, 1H), 2.74-2.67 (m, 4H), 1.65 (d, J=7.2 Hz, 3H), 1.30 (d, J=6.4 Hz, 3H). ¹⁹F NMR (376 MHz, CDCl₃) δ −115.81.

Compound 36B

LC-MS (ESI): R_{T}=3.063 min, mass calcd. for C₂₇H₂₆Cl₂F₂N₆O₃ 590.1, m/z found 591.2 [M+H]⁺. Chiral analysis (Column: Superchiral IB N-5, 5 μm 4.6*250 mm; Mobile Phase: 100% CH₃CN at 1.0 mL/min; Temp: 30° C.; Wavelength: 254 nm, Back pressure: 100 bar; R_{T}=6.219 min). ¹H NMR (400 MHz, CDCl₃) δ 8.67 (s, 1H), 7.84 (d, J=6.8 Hz, 1H), 7.63 (d, J=8.0 Hz, 1H), 7.54-7.52 (m, 2H), 7.28-7.25 (m, 1H), 6.63 (t, J=55.2 Hz, 1H), 6.19-5.28 (m, 3H), 4.95-4.31 (m, 3H), 4.19-4.15 (m, 1H), 3.45 (dd, J=° 13.2, 4.8 Hz, 1H), 3.15-2.95 (m, 1H), 2.81 (d, J=4.8 Hz, 3H), 2.73-2.69 (m, 1H), 1.66 (d, J=7.2 Hz, 3H), 1.29 (d, J=6.8 Hz, 3H). ¹⁹F NMR (376 MHz, CDCl₃) δ −115.73.

Compounds 37A and 37B

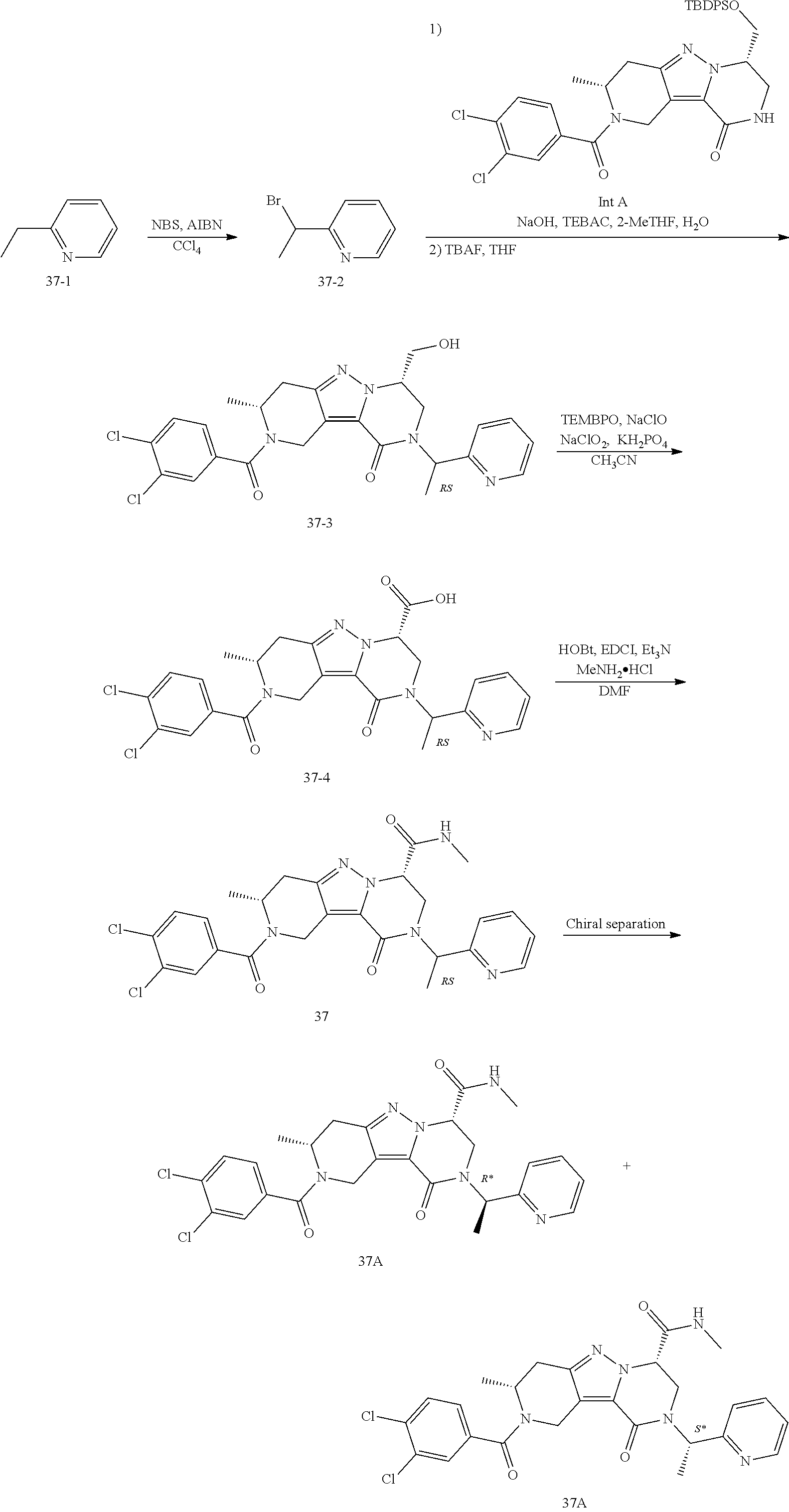

Intermediate 37-2

2-(1-Bromoethyl)pyridine

To a solution of 2-ethylpyridine 37-1 (2.0 g, 18.7 mmol), N-bromosuccinimide (3.7 g, 20.8 mmol) and 2,2′-azobis(2-methylpropionitrile) (307 mg, 1.87 mmol) in carbon tetrachloride (50 mL). After being stirred at 90° C. for 1 hour, the reaction mixture was diluted with water (30 mL) and extracted with ethyl acetate (30 mL) twice. The combined organic layers were washed with water (30 mL) for three times, brine (30 mL), dried over Na₂SO₄₍ₛ₎ and filtered. The filtrate was concentrated under reduced pressure to give crude, which was purified by silica gel column chromatography (petroleum ether:ethyl acetate=100:1 to 50:1) to give the title compound (2.4 g, 90% purity from ¹H NMR, 62% yield) as red oil. ¹H NMR (400 MHz, CDCl₃) δ 8.58-8.57 (m, 1H), 7.71-7.67 (m, 1H), 7.46-7.44 (m, 1H), 7.22-7.18 (m, 1H), 5.24 (q, J=7.2 Hz, 1H), 2.08 (d, J=6.8 Hz, 3H)

Intermediate 37-3

(3R,7S)-2-(3,4-Dichlorobenzoyl)-7-(hydroxymethyl)-3-methyl-9-(1-(pyridin-2-yl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichloro benzoyl)-3-methyl-1,2,3,4,8,9-hexahydropyrido[4′,3′:3,4]pyrazolo[1,5-a]pyrazin-10(7H)-one Int A (1.5 g, 100% purity, 2.32 mmol) and 2-(1-bromoethyl)pyridine 37-2 (957 mg, 90% purity, 4.63 mmol) in 2-methyltetrahydrofuran (15 mL) was added 50% wt. sodium hydroxide aqueous solution (15 mL) and benzyltriethylammonium chloride (106 mg, 0.465 mmol). After being stirred at 20° C. for 3 hours, the mixture was added into water (50 mL) and extracted with dichloromethane (50 mL) twice. The combined organic layers were washed with water (50 mL), brine (50 mL), dried over Na₂SO₄₍ₛ₎ and filtered. The filtrate was concentrated under reduced pressure give a residue, which was diluted with tetrahydrofuran (20 mL), 1M tetrabutylammonium fluoride in tetrahydrofuran (2.3 mL, 2.3 mmol) was added into the solution. After being stirred at 20° C. for 3 hours. the reaction mixture was diluted with water (30 mL) and extracted with ethyl acetate (30 mL) twice. The combined organic layers were washed with water (30 mL) for three times, brine (30 mL), dried over Na₂SO₄₍ₛ₎ and filtered. The filtrate was concentrated to give a residue, which was purified by C18 column (acetonitrile:water=5% to 95%) to give the title compound (700 mg, 96% purity from LCMS, 70% yield) as yellow oil. LC-MS (ESI): R_{T}=1.319 min, mass calcd. for C₂₅H₂₅Cl₂N₅O₃ 513.1 m/z found 514.2 [M+H]⁺.

Intermediate 37-4

(3R,7S)-2-(3,4-Dichlorobenzoyl)-3-methyl-10-oxo-9-(1-(pyridin-2-yl)ethyl)-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-7-(hydroxymethyl)-3-methyl-9-(1-(pyridin-2-yl)ethyl)-1,2,3,4,8,9-hexahydropyrido[4′,3′:3,4]pyrazolo[1,5-a]pyrazin-10(7H)-one 37-3 (700 mg, 96% purity, 1.31 mmol) in acetonitrile (10 mL) was added saturated potassium dihydrogenphosphate aqueous solution (10 mL), sodium chlorite (300 mg, 80% purity, 2.65 mmol), 2,2,6,6-tetramethylpiperidinooxy (410 mg, 2.62 mmol) and sodium hypochlorite aqueous solution (1.6 mL, 2.69 mmol) at 0° C. After being stirring at 0° C. to 20° C. for 14 hours, the reaction mixture was diluted with 0.1 M hydrochloride aqueous solution (40 mL) and extracted with ethyl acetate (40 mL) twice. The combined organic layers were washed with brine (40 mL) and dried over Na₂SO₄₍ₛ₎ and filtered. The filtrate was concentrated to give a residue, which was purified by C18 column (acetonitrile:water=45% to 60%) to give the title compound (400 mg, 99% purity from LCMS, 57% yield) as white solids. LC-MS (ESI): R_{T}=1.126 min, mass calcd. for C₂₅H₂₃Cl₂N₅O₄ 527.1 m/z found 528.0 [M+H]⁺.

Compound 37

(3R,7S)-2-(3,4-Dichlorobenzoyl)-N,3-dimethyl-10-oxo-9-(1-(pyridin-2-yl)ethyl)-1,2,3,4,7,8,9,10-octahydropyrido[4′,3′:3,4]pyrazolo[1,5-a]pyrazine-7-carboxamide

To a solution of (3R,7S)-2-(3,4-dichlorobenzoyl)-3-methyl-10-oxo-9-(1-(pyridin-2-yl)ethyl)-1,2,3,4,7,8,9,10-octahydropyrido[4′,3′:3,4]pyrazolo[1,5-a]pyrazine-7-carboxylic acid 37-4 (400 mg, 99% purity, 0.749 mmol) in N,N-dimethylformamide (15 mL) was added methylamine hydrochloride (127 mg, 1.88 mmol), benzotriazol-1-ol (203 mg, 1.50 mmol), 1-(3-dimethylaminopropyl)-3-ethylcarbodiimide hydrochloride (287 mg, 1.50 mmol) and triethylamine (530 mg, 5.24 mmol) at 0° C. After being stirred at 0° C. for 2 hours, the mixture was acidified to pH=6 with 0.05 M hydrochloride aqueous solution and extracted with ethyl acetate (60 mL) twice. The combined organic layers were washed with water (60 mL) for three times and brine (20 mL), dried over Na₂SO₄₍ₛ₎ and filtered. The filtrate was concentrated under reduced pressure to give a residue. It was purified by C18 column (acetonitrile:water=55% to 60%) to give the title compound (300 mg, 100% purity, 74% yield) as white solids. LC-MS (ESI): R_{T}=1.45 min and 1.47 min, mass calcd. for C₂₆H₂₆Cl₂N₆O₃ 540.1 m/z found 541.3 [M+H]⁺.

Compounds 37A and 37B

(3R,7S)-2-(3,4-Dichlorobenzoyl)-N,3-dimethyl-10-oxo-9-((R*)-1-(pyridin-2-yl)ethyl)-1,2,3,4,7,8,9,10-octahydropyrido[4′,3′:3,4]pyrazolo[1,5-a]pyrazine-7-carboxamide (37A), and (3R,7S)-2-(3,4-dichlorobenzoyl)-N,3-dimethyl-10-oxo-9-((S*)-1-(pyridin-2-yl)ethyl)-1,2,3,4,7,8,9,10-octahydropyrido[4′,3′:3,4]pyrazolo[1,5-a]pyrazine-7-carboxamide (37B)

(3R,7S)-2-(3,4-dichlorobenzoyl)-N,3-dimethyl-10-oxo-9-(1-(pyridin-2-yl)ethyl)-1,2,3,4,7,8,9,10-octahydropyrido[4′,3′:3,4]pyrazolo[1,5-a]pyrazine-7-carboxamide 37 (300 mg, 100% purity, 0.554 mmol) was separated by chiral Prep. HPLC (separation condition: Column: Chiralpak IH 5 μm 20*250 mm; Mobile Phase: CAN at 12 mL/min; Temp: 30° C.; Wavelength: 214 nm) to afford the title compound 37-A (41.8 mg, 97.3% purity, 14% yield, 100% stereopure) as white solids and compound 37-B (66.4 mg, 98.5% purity, 22% yield, 100% stereopure) as white solids.

Compound 37A

LC-MS (ESI): R_{T}=3.346 min, mass calcd. for C₂₆H₂₆Cl₂N₆O₃ 540.1 m/z found 541.2 [M+H]⁺. Chiral analysis (Column: Superchiral IH 5 μm 4.6*250 mm; Mobile Phase: ACN 100% at 1 mL/min; Temp: 30° C.; Wavelength: 254 nm; Rt=6.089 min)¹H NMR (400 MHz, CDCl₃) δ 8.58 (d, J=4.4 Hz, 1H), 7.67-7.63 (m, 1H), 7.54-7.50 (m, 2H), 7.25-7.21 (m, 3H), 5.93-5.45 (m, 3H), 4.89-4.37 (m, 3H), 4.13-3.97 (m, 2H), 3.03 (br s, 1H), 2.73-2.69 (m, 1H), 2.65 (d, J=4.8 Hz, 3H), 1.65 (d, J=7.2 Hz, 3H), 1.29 (d, J=6.8 Hz, 3H).

Compound 37B

LC-MS (ESI): R_{T}=3.476 min, mass calcd. for C₂₆H₂₆Cl₂N₆O₃ 540.1 m/z found 541.2 [M+H]⁺. Chiral analysis (Column: Superchiral IH 5 μm 4.6*250 mm; Mobile Phase: ACN 100% at 1 mL/min; Temp: 30° C.; Wavelength: 254 nm; Rt=7.514 min). ¹H NMR (400 MHz, CDCl₃) δ 8.55 (d, J=4.0 Hz, 1H), 7.68-7.64 (m, 1H), 7.52-7.49 (m, 2H), 7.34-7.32 (m, 1H), 7.25-7.17 (m, 2H), 6.03-5.30 (m, 3H), 4.86-4.31 (m, 4H), 3.80-3.73 (m, 1H), 3.02 (br s, 1H), 2.81 (d, J=4.4 Hz, 3H), 2.73-2.67 (m, 1H), 1.64 (d, J=6.8 Hz, 3H), 1.30 (d, J=6.8 Hz, 3H).

Compounds 38A and 38B

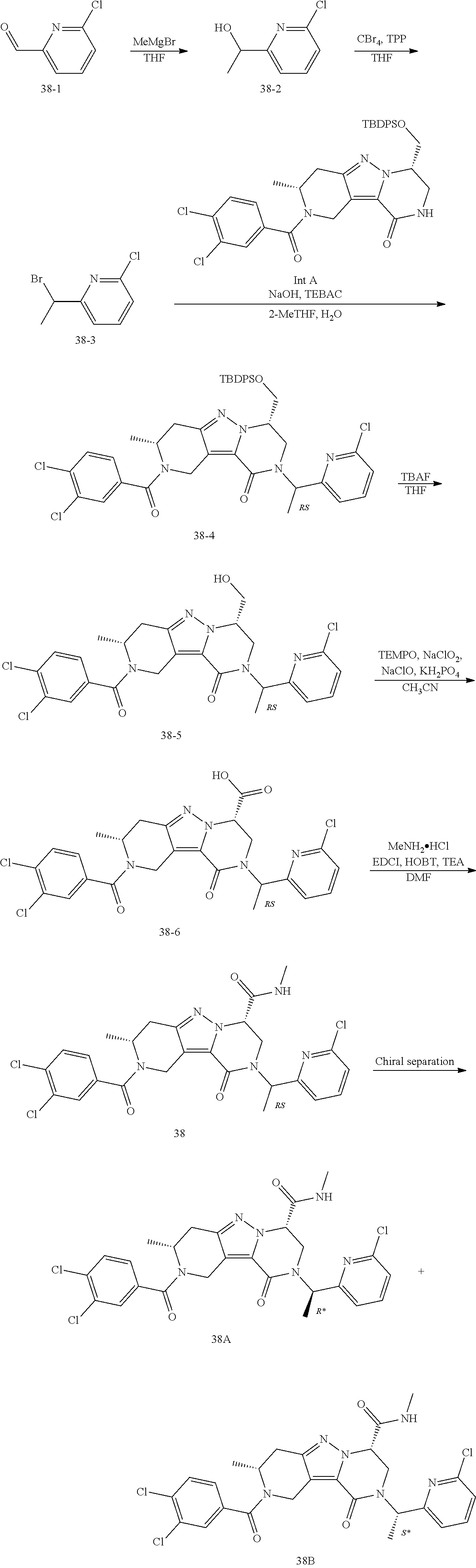

Intermediate 38-2

1-(6-Chloropyridin-2-yl)ethan-1-ol

To the solution of 6-chloropicolinaldehyde 38-1 (1.0 g, 7.06 mmol) in tetrahydrofuran (10 mL) was added 1 M methylmagnesium bromide in 2-methyltetrahydrofuran (10 mL, 10 mmol) at 0° C. After being stirred at 0° C. for 1 hour, the mixture was quenched with ammonium chloride aqueous solution (20 mL) and extracted with ethyl acetate (20 mL) twice. The combined organic layers were washed with brine (35 mL), dried over Na₂SO₄₍ₛ₎ and filtered. The filtrate was concentrated to give the title compound (1.0 g, 98% purity, 88% yield) as yellow oil. LC-MS (ESI): R_{T}=1.35 min, mass calcd. for C₇H₈ClNO 157.0, m/z found 158.3 [M+H]⁺.

Intermediate 38-3

2-(1-Bromoethyl)-6-chloropyridine

To a solution of 1-(6-chloropyridin-2-yl)ethanol 38-2 (150 mg, 98% purity, 0.933 mmol) in tetrahydrofuran (2 mL) were added triphenylphosphine (441 mg, 1.33 mmol) and perbromomethane (441 mg, 1.68 mmol) at 0° C. After being stirred at 25° C. for 4 hours, the mixture was filtered. The filtrate was concentrated and purified by column chromatography on silica gel (petroleum ether:ethyl acetate=5:1) to give the title compound (190 mg, 100% purity, 92.4% yield) as yellow oil. LC-MS (ESI): R_{T}=1.62 min, mass calcd. for C₇H₇BrClN 218.9, m/z found 220.0 [M+H]⁺.

Intermediate 38-4

(3R,7S)-7-(((tert-Butyldiphenylsilyl)oxy)methyl)-9-(1-(6-chloropyridin-2-yl)ethyl)-2-(3,4-dichlorobenzoyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (420 mg, 90% purity, 0.584 mmol) in 2-methyltetrahydrofuran (4 mL) and 50% wt. sodium hydroxide in water (4 mL, 125.0 mmol) was added 2-(1-bromoethyl)-6-chloropyridine 38-3 (190 mg, 100% purity, 0.862 mmol) and benzyltriethylammonium chloride (21 mg, 0.092 mmol) at room temperature. After being stirred at room temperature for 2 hours, the reaction mixture was quenched with water (10 mL) and extracted with ethyl acetate (10 mL) twice. The combined organic layers were washed with brine (10 mL), dried over Na₂SO₄₍ₛ₎ and filtered. The filtrate was concentrated and purified by C18 column (acetonitrile:water=50% to 60%) to give the title compound (400 mg, 88% purity, 76.6% yield) as white solids. LC-MS (ESI): R_{T}=2.05 min and 2.10 min, mass calcd. for C₄₁H₄₂C₁₃N₅O₃Si 785.2, m/z found 786.1 [M+H]⁺.

Intermediate 38-5

(3R,7S)-9-(1-(6-Chloropyridin-2-yl)ethyl)-2-(3,4-dichlorobenzoyl)-7-(hydroxymethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-9-(1-(6-chloropyridin-2-yl)ethyl)-2-(3,4-dichlorobenzoyl)-3-methyl-1,2,3,4,8,9-hexahydropyrido[4′,3′:3,4]pyrazolo[1,5-a]pyrazin-10(7H)-one 38-4 (400 mg, 88% purity, 0.447 mmol) in tetrahydrofuran (6 mL) was added 1 M tetrabutylammonium fluoride in tetrahydrofuran (0.8 mL, 0.8 mmol) at room temperature. After being stirred at room temperature for 1 hour, the reaction mixture was quenched with water (10 mL) and extracted with ethyl acetate (10 mL) twice. The combined organic layers were washed with brine (20 mL) twice, dried over Na₂SO₄₍ₛ₎ and filtered. The filtrate was concentrated and purified by column chromatography on silica gel (dichloromethane:ethyl acetate=10:1) to give desired compound (230 mg, 100% purity, 93.7% yield) as white solids. LC-MS (ESI): R_{T}=1.60 min, mass calcd. for C₂₅H₂₄C₁₃N₅O₃ 547.1, m/z found 548.4 [M+H]⁺.

Intermediate 38-6

(3R,7S)-9-(1-(6-Chloropyridin-2-yl)ethyl)-2-(3,4-dichlorobenzoyl)-3-methyl-10-oxo-1,2,3,4,7,8,9,10-octahydropyrido[4′,3′:3,4]pyrazolo[1,5-a]pyrazine-7-carboxylic acid

To a solution of (3R)-9-(1-(6-chloropyridin-2-yl)ethyl)-2-(3,4-dichlorobenzoyl)-7-(hydroxymethyl)-3-methyl-1,2,3,4,8,9-hexahydropyrido[4′,3′:3,4]pyrazolo[1,5-a]pyrazin-10(7H)-one 38-5 (280 mg, 100% purity, 0.510 mmol), sodium chlorite (115 mg, 1.02 mmol) and 2,2,6,6-tetramethylpiperidinooxy (160 mg, 1.02 mmol) in acetonitrile (3 mL) and saturated potassium dihydrogenphosphate aqueous solution (3 mL) was added sodium hypochlorite aqueous solution (0.6 mL, 1.01 mmol) at 0° C. After being stirred at room temperature overnight, the reaction was quenched with saturated sodium sulfite aqueous solution (10 mL), acidized with 1 M hydrochloride to pH˜4 and extracted with ethyl acetate (15 mL) twice. The combined organic layers were dried over Na₂SO₄₍ₛ₎ and filtered. The filtrate was concentrated and purified by C18 column (acetonitrile:water=50% to 60%) to give the title compound (250 mg, 100% purity from LCMS, 87.1% yield) as white solids. LC-MS (ESI): R_{T}=1.33 min, mass calcd. for C₂₅H₂₂C₁₃N₅O₄ 561.1, m/z found 562.4 [M+H]⁺.

Compound 38

(3R,7S)-9-(1-(6-Chloropyridin-2-yl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide

To a solution of (3R,7S)-9-(1-(6-Chloropyridin-2-yl)ethyl)-2-(3,4-dichlorobenzoyl)-3-methyl-10-oxo-1,2,3,4,7,8,9,10-octahydropyrido[4′,3′:3,4]pyrazolo[1,5-a]pyrazine-7-carboxylic acid 38-6 (250 mg, 100% purity, 0.444 mmol), 1-(3-dimethylaminopropyl)-3-ethylcarbodiimide hydrochloride (170 mg, 0.887 mmol), methylamine hydrochloride (70 mg, 1.04 mmol) and 1H-benzo[d][1,2,3]triazol-1-ol (125 mg, 0.925 mmol) in N,N-dimethylformamide (3 mL) was added triethylamine (0.5 mL, 2.82 mmol) at 0° C. After being stirred at room temperature for 2 hours, the mixture was quenched with ammonium chloride aqueous solution (10 mL) and extracted with ethyl acetate (10 mL) twice. The combined organic layers were washed with brine (20 mL), dried over Na₂SO₄₍ₛ₎ and filtered. The filtrate was concentrated and purified by C18 column (acetonitrile:water (0.1% ammonium bicarbonate=45% to 55%) to give the title compound (200 mg, 100% purity, 78.2% yield) as yellow solids. LC-MS (ESI): R_{T}=1.60 min, mass calcd. for C₂₆H₂₅C₁₃N₆O₃ 574.1, m/z found 575.2 [M+H]⁺.

Compounds 38A and 38B

(3R,7S)-9-((R*)-1-(6-Chloropyridin-2-yl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide (38A), and (3R,7S)-9-((S*)-1-(6-chloropyridin-2-yl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide (38B)

(3R,7S)-9-(1-(6-chloropyridin-2-yl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide 38 (260 mg, 100% purity, 0.451 mmol) was separated by chiral Prep. (Column: Chiralpak IC 5 μm 30*250 mm; Mobile Phase: MeOH:EtOH=50:50 at 25 mL/min; Temp: 30° C.; Wavelength: 254 nm) to give the compound 38A (59.6 mg, 99.3% purity, 22.8% yield, 100% stereopure) as white solids and compound 38B (62.9 mg, 99.5% purity, 24.1% yield, 99.9% stereopure) as white solids.

Compound 38A

LC-MS (ESI): R_{T}=2.349 min, mass calcd. for C₂₆H₂₅C₁₃N₆O₃ 574.1, m/z found 575.1 [M+H]⁺. Chiral analysis (Column: Chiralpak IC 5 μm 4.6*250 mm; Mobile Phase: MeOH:EtOH=50:50 at 1 mL/min; Temp: 30° C.; Wavelength: 254 nm, R_{T}=6.049 min). ¹H NMR (400 MHz, CDCl₃) δ 7.64-7.59 (m, 1H), 7.56-7.48 (m, 2H), 7.30-7.26 (m, 1H), 7.26-7.23 (m, 1H), 7.21-7.15 (m, 1H), 6.06-5.40 (m, 3H), 4.90-4.37 (m, 3H), 4.22-4.12 (m, 1H), 4.03-3.94 (m, 1H), 3.18-2.96 (m, 1H), 2.77-2.62 (m, 4H), 1.63 (d, J=6.8 Hz, 3H), 1.38-1.22 (m, 3H).

Compound 38B

LC-MS (ESI): R_{T}=2.515 min, mass calcd. for C₂₆H₂₅C₁₃N₆O₃ 574.1, m/z found 575.1 [M+H]⁺. Chiral analysis (Column: Chiralpak IC 5 μm 4.6*250 mm; Mobile Phase: MeOH:EtOH=50:50 at 1 mL/min; Temp: 30° C.; Wavelength: 254 nm, R_{T}=7.141 min). ¹H NMR (400 MHz, CDCl₃) δ 7.64-7.59 (m, 1H), 7.54-7.47 (m, 2H), 7.25-7.20 (m, 3H), 6.10-5.72 (m, 2H), 5.63-5.23 (m, 1H), 5.03-4.62 (m, 2H), 4.52-4.20 (m, 2H), 3.96-3.86 (m, 1H), 3.18-2.96 (m, 1H), 2.81 (d, J=4.8 Hz, 3H), 2.76-2.64 (m, 1H), 1.62 (d, J=6.8 Hz, 3H), 1.34-1.25 (m, 3H).

Compounds 39A and 39B

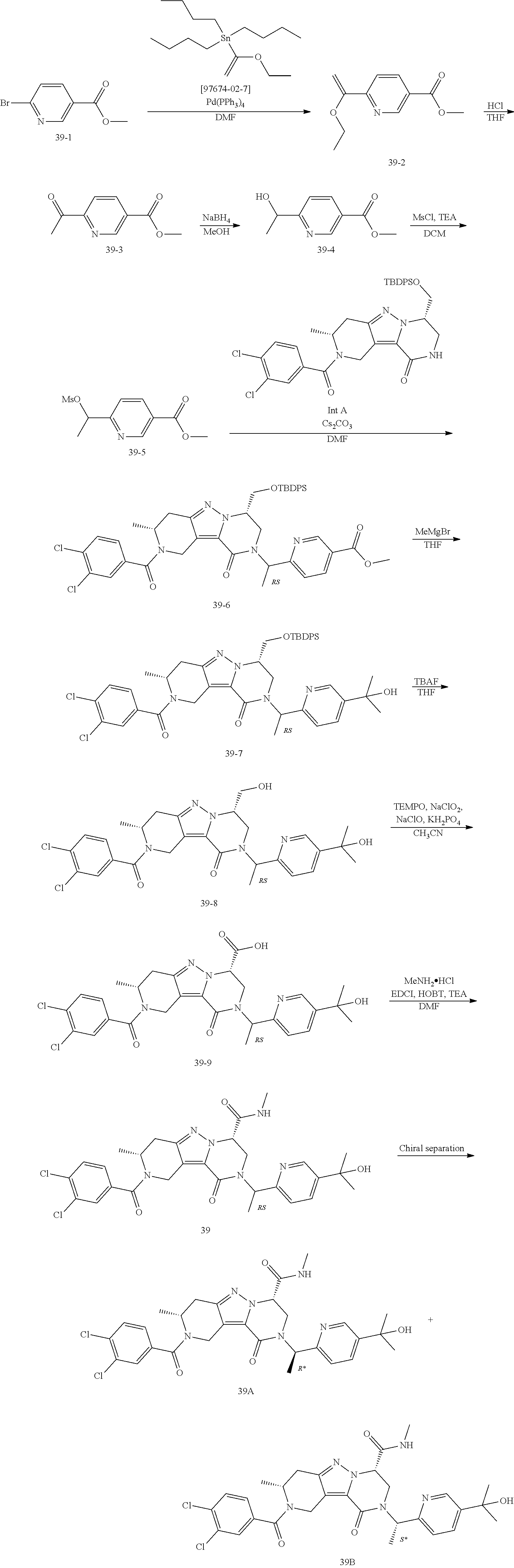

Intermediate 39-2

Methyl 6-(1-ethoxyvinyl)nicotinate

To a solution of methyl 6-bromonicotinate 39-1 (5.00 g, 23.1 mmol) in N,N-dimethylformamide (60 mL) was added tetrakis(triphenylphosphine)palladium (1.40 g, 1.21 mmol) and tributyl(1-ethoxyvinyl)stannane (10 mL, 29.6 mmol) at room temperature under nitrogen atmosphere. After being stirred at 100° C. overnight under nitrogen atmosphere, the mixture was diluted with saturated potassium fluoride aqueous solution (100 mL) and filtered. The filtrate was extracted with ethyl acetate (100 mL) for three times. The combined organic layers were washed with brine (100 mL), dried over Na₂SO₄₍ₛ₎ and filtered. The filtrate was concentrated and purified by silica gel column chromatography (petroleum ether:ethyl acetate=20:1) to give the title compound (3.30 g, 90% purity from ¹H NMR, 62% yield) as light yellow solids. LC-MS (ESI): R_{T}=1.65 min, mass calcd. for C¹¹H₁₃NO₃ 207.1, m/z found 208.4 [M+H]⁺. ¹H NMR (300 MHz, CDCl₃) δ 9.24-9.21 (m, 1H), 8.37-8.33 (m, 1H), 7.83 (d, J=8.4 Hz, 1H), 5.66-5.63 (m, 1H), 4.60-4.55 (m, 1H), 4.10-4.02 (m, 5H), 1.53 (t, J=6.9 Hz, 3H).

Intermediate 39-3

Methyl 6-acetylnicotinate

To a solution of methyl 6-(1-ethoxyvinyl)nicotinate 39-2 (3.30 g, 90% purity, 14.3 mmol) in tetrahydrofuran (30 mL) was added 2 M hydrochloride aqueous solution (30 mL, 60.0 mmol) at room temperature under nitrogen atmosphere. After being stirred at room temperature for 1 hour, the reaction mixture was added saturated sodium bicarbonate aqueous solution (100 mL) and extracted with ethyl acetate (50 mL) for three times. The combined organic layers were washed with brine (100 mL) then dried Na₂SO₄₍ₛ₎, concentrated to give the title compound (2.80 g, 90% purity from ¹H NMR, 98% yield) as white solids. LC-MS (ESI): R_{T}=1.38 min, mass calcd. for C₉H₉NO₃ 179.1, m/z found 180.7 [M+H]⁺. ¹H NMR (300 MHz, CDCl₃) δ 9.31 (s, 1H), 8.49-8.45 (m, 1H), 8.15 (d, J=9.9 Hz, 1H), 4.04 (s, 3H), 2.81 (s, 3H).

Intermediate 39-4

Methyl 6-(1-hydroxyethyl)nicotinate

To the solution of methyl 6-acetylnicotinate 39-3 (7.60 g, 57.5% purity, 24.4 mmol) in methanol (200 mL) was added sodium borohydride (1.10 g, 29.1 mmol) at 0° C. After being stirred at room temperature for 2 hours, the mixture was quenched with saturated ammonium chloride aqueous solution (50 mL), then extracted with ethyl acetate (100 mL) for three times, dried over Na₂SO₄₍ₛ₎ and filtered. The filtrate was concentrated to give the title compound (3.50 g, 90% purity from ¹H NMR, 71.3% yield) as yellow oil. LC-MS (ESI): R_{T}=1.17 min, mass calcd. for C₉H₁₁NO₃ 181.1, m/z found 182.1 [M+H]⁺. ¹H NMR (300 MHz, CDCl₃) δ 9.15 (d, J=J=1.2 Hz, 1H), 8.31 (dd, J=2.1 Hz, 8.4 Hz, 1H), 7.43 (d, J=8.4 Hz, 1H), 5.01-4.95 (m, 1H), 4.22-4.11 (m, 1H), 3.98 (s, 3H), 1.55 (d, J=6.6 Hz, 3H).

Intermediate 39-5

Methyl 6-(1-((methylsulfonyl)oxy)ethyl)nicotinate

To a solution of methyl 6-(1-hydroxyethyl)nicotinate 39-4 (3.50 g, 90% purity, 8.94 mmol) in dichloromethane (18 mL) was added triethylamine (5.20 g, 51.4 mmol) and methanesulfonyl chloride (3.00 g, 26.2 mmol) at 0° C. under nitrogen atmosphere. After being stirred at room temperature for 3 hours, the reaction mixture was added saturated sodium bicarbonate aqueous solution (40 mL) and extracted with dichloromethane (40 mL) for three times. The combined organic layers were washed with brine (50 mL) then dried Na₂SO₄₍ₛ₎, concentrated to give the title compound (4.40 g, 90% purity from ¹H NMR, 87.9% yield) as yellow solids. LC-MS (ESI): R_{T}=1.37 min, mass calcd. for C₁₀H₁₃NO₅S 259.1, m/z found 260.0 [M+H]⁺. ¹H NMR (300 MHz, CDCl₃) δ 9.21 (d, J=1.5 Hz, 1H), 8.38 (dd, J=8.1 and 2.1 Hz, 1H), 7.60 (d, J=8.4 Hz, 1H), 5.86 (d, J=6.6 Hz, 1H), 3.99 (s, 3H), 3.04 (s, 3H), 1.80 (d, J=6.6 Hz, 3H).

Intermediate 39-6

Methyl 6-(1-((3R,7S)-7-(((tert-butyldiphenylsilyl)oxy)methyl)-2-(3,4-dichlorobenzoyl)-3-methyl-10-oxo-1,2,3,4,7,8-hexahydropyrido[4′,3′:3,4]pyrazolo[1,5-a]pyrazin-9(10H)-yl)ethyl)nicotinate

To a mixture of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (500 mg, 90% purity, 0.695 mmol) and methyl 6-(1-((methylsulfonyl)oxy)ethyl)nicotinate 39-5 (900 mg, 3.12 mmol) in N,N-dimethylformamide (10 mL) was added cesium carbonate (1.30 g, 3.99 mmol). After being stirred at 50° C. for 3 hours, the mixture was diluted with water (50 mL) and extracted with ethyl acetate (20 mL) twice. The combined organic layers were washed with brine (10 mL), dried over Na₂SO₄₍ₛ₎ and filtered. The filtrate was concentrated and purified by C18 column (acetonitrile:water (0.1% ammonium bicarbonate)=50%-95%) to give the title compound (270 mg, 92.0% purity from LCMS, 44.1% yield) as yellow solids. LC-MS (ESI): R_{T}=2.25 min and 2.34 min, mass calcd. for C₄₃H₄₅Cl₂N₅O₅Si 809.3, m/z found 810.2 [M+H]⁺.

Intermediate 39-7

(3R,7S)-7-(((tert-Butyldiphenylsilyl)oxy)methyl)-2-(3,4-dichlorobenzoyl)-9-(1-(5-(2-hydroxypropan-2-yl)pyridin-2-yl)ethyl)-3-methyl-1,2,3,4,8,9-hexahydropyrido[4′,3′: 3,4]pyrazolo[1,5-a]pyrazin-10(7H)-one

To the solution of methyl 6-(1-((3R,7S)-7-(((tert-butyldiphenylsilyl)oxy)methyl)-2-(3,4-dichlorobenzoyl)-3-methyl-10-oxo-1,2,3,4,7,8-hexahydropyrido[4′,3′:3,4]pyrazolo[1,5-a]pyrazin-9(10H)-yl)ethyl)nicotinate 39-6 (600 mg, 90% purity, 0.667 mmol) in tetrahydrofuran (10 mL) was added 1M methylmagnesium bromide in tetrahydrofuran (4 mL, 4.00 mmol) at −30° C. The mixture was stirred at −30° C. for 2 hours. The mixture was quenched with saturated ammonium chloride aqueous solution (10 mL) and extracted with ethyl acetate (50 mL) twice. The combined organic layers were washed with brine (50 mL), dried over Na₂SO₄₍ₛ₎ and filtered. The filtrate was concentrated to give the title compound (650 mg, 59.8% purity from LCMS, 72.0% yield) as yellow solids. LC-MS (ESI): R_{T}=1.70 min and 1.78 min, mass calcd. for C₄₄H₄₉Cl₂N₅O₄Si 809.3, m/z found 810.2 [M+H]⁺.

Intermediate 39-8

(3R,7S)-2-(3,4-Dichlorobenzoyl)-7-(hydroxymethyl)-9-(1-(5-(2-hydroxypropan-2-yl)pyridin-2-yl)ethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-9-(1-(5-(2-hydroxypropan-2-yl)pyridin-2-yl)ethyl)-3-methyl-1,2,3,4,8,9-hexahydropyrido[4′,3′:3,4]pyrazolo[1,5-a]pyrazin-10(7H)-one 39-7 (600 mg, 93.8% purity, 0.694 mmol) in tetrahydrofuran (10 mL) was added 1 M tetrabutylammonium fluoride (2 mL, 2 mmol) at 0° C. Then the reaction solution was stirred at room temperature under nitrogen atmosphere for 3 hours. The mixture was diluted with water (50 mL) and extracted with ethyl acetate (20 mL) twice. The combined organic layers were washed with brine (10 mL), dried over Na₂SO₄₍ₛ₎ and filtered. The filtrate was concentrated and purified by C18 column (acetonitrile:water (0.1% ammonium bicarbonate)=5%-95%) to give the title compound (300 mg, 91.7% purity from LCMS, 69.2% yield) as yellow oil. LC-MS (ESI): R_{T}=1.44 min and 1.47 min, mass calcd. for C₂₈H₃₁Cl₂N₅O₄ 571.2, m/z found 572.1 [M+H]⁺.

Intermediate 39-9

(3R,7S)-2-(3,4-Dichlorobenzoyl)-9-(1-(5-(2-hydroxypropan-2-yl)pyridin-2-yl)ethyl)-3-methyl-10-oxo-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-7-(hydroxymethyl)-9-(1-(5-(2-hydroxypropan-2-yl)pyridin-2-yl)ethyl)-3-methyl-1,2,3,4,8,9-hexahydropyrido[4′,3′:3,4]pyrazolo[1,5-a]pyrazin-10(7H)-one 39-8 (250 mg, 91.7% purity, 0.400 mmol) in acetonitrile (2.5 mL) was added saturated potassium dihydrogenphosphate aqueous solution (2.5 mL), sodium chlorite (75 mg, 80% purity, 0.663 mmol), 2,2,6,6-tetramethylpiperidinooxy (125 mg, 0.800 mmol) and sodium hypochlorite aqueous solution (0.75 mL, 10% purity, 1.26 mmol) at 0° C. After being stirred at 20° C. for 16 hours, the reaction mixture was filtered, washed by acetonitrile (10 mL), then quenched with saturated sodium sulfite aqueous solution (1 mL), acidized with 1 M hydrochloride aqueous solution to pH˜4, extracted with ethyl acetate (20 mL) twice. The combined organic layers were washed with brine (20 mL), dried over Na₂SO₄₍ₛ₎, and filtered. The filtrate was concentrated to get a residue, which was purified by C18 column (acetonitrile:water (0.1% ammonium bicarbonate)=5%-95%) to give the title compound (170 mg, 90.3% purity from LCMS, 65.4% yield) as white solids. LC-MS (ESI): R_{T}=1.23 min, mass calcd. for C₂₈H₂₉Cl₂N₅O₅ 585.2, m/z found 586.1 [M+H]⁺.

Compound 39

(3R,7S)-2-(3,4-Dichlorobenzoyl)-9-(1-(5-(2-hydroxypropan-2-yl)pyridin-2-yl)ethyl)-N,3-dimethyl-10-oxo-1,2,3,4,7,8,9,10-octahydropyrido[4′,3′:3,4]pyrazolo[1,5-a]pyrazine-7-carboxamide

To a solution of (3R,7S)-2-(3,4-dichlorobenzoyl)-9-(1-(5-(2-hydroxypropan-2-yl)pyridin-2-yl)ethyl)-3-methyl-10-oxo-1,2,3,4,7,8,9,10-octahydropyrido[4′,3′:3,4]pyrazolo[1,5-a]pyrazine-7-carboxylic acid 39-9 (100 mg, 90% purity, 0.153 mmol), methanamine hydrochloride (30 mg, 0.444 mmol), 1-(3-dimethylaminopropyl)-3-ethylcarbodiimide hydrochloride (60 mg, 0.313 mmol) and 1-hydroxybenzotriazole (45 mg, 0.333 mmol) in N,N-dimethylformamide (2 mL) was slowly added triethylamine (100 mg, 0.988 mmol) at 0° C. After being stirred at 0° C. under nitrogen atmosphere for 3 hours. The reaction mixture was quenched with water (10 mL) and extracted with ethyl acetate (10 mL) for three times.

The combined organic layers were dried over Na₂SO₄₍ₛ₎ and filtered. The filtrate was concentrated and purified by C18 column (acetonitrile:water=5% to 100%) to give the title compound (50 mg, 90% purity from ¹H NMR, 48.9% yield) as white solids. LC-MS (ESI): R_{T}=1.60 min and 1.62 min, mass calcd. for C₂₉H₃₂Cl₂N₆O₄ 598.2, m/z found 599.2 [M+H]⁺.

Compounds 39A and 39B

(3R,7S)-2-(3,4-Dichlorobenzoyl)-9-((R*)-1-(5-(2-hydroxypropan-2-yl)pyridin-2-yl) ethyl)-N,3-dimethyl-10-oxo-1,2,3,4,7,8,9,10-octahydropyrido[4′,3′:3,4]pyrazolo[1,5-a]pyrazine-7-carboxamide (39A), and (3R,7S)-2-(3,4-dichlorobenzoyl)-9-((S*)-1-(5-(2-hydroxypropan-2-yl)pyridin-2-yl)ethyl)-N,3-dimethyl-10-oxo-1,2,3,4,7,8,9,10-octahydropyrido[4′,3′:3,4]pyrazolo[1,5-a]pyrazine-7-carboxamide (39B)

(3R,7S)-2-(3,4-dichlorobenzoyl)-9-(1-(5-(2-hydroxypropan-2-yl)pyridin-2-yl)ethyl)-N,3-dimethyl-10-oxo-1,2,3,4,7,8,9,10-octahydropyrido[4′,3′:3,4]pyrazolo[1,5-a]pyrazine-7-carboxamide 39 (90 mg, 90% purity, 0.135 mmol) was separated by Chiral Prep-HPLC (Column: Chiralpak IE 10 μm 30 mm*250 mm; Mobile Phase: ACN:IPA=80:20 at 30 mL/min; Col. Temp: 30° C.; Wavelength: 254 nm) to afford the title compound 39A (20 mg, 99.8% purity from LCMS, 24.6% yield, 100% stereopure) as white solids and compound 39B (17 mg, 99.5% purity from LCMS, 20.9% yield, 99.2% stereopure) as white solids.

Compound 39A

LC-MS (ESI): R_{T}=3.278 min, mass calcd. for C₂₉H₃₂Cl₂N₆O₄ 598.2, m/z found 599.2 [M+H]⁺. Chiral analysis (Column: Chiralpak IE; Column size: 5 μm 4.6 mm*250 mm; Mobile Phase: Hexane:IPA=80:20 at 30 mL/min; Col. Temp: 30° C.; Wave length: 254 nm; Rt=8.865 min). ¹H NMR (400 MHz, CDCl₃) δ 8.70 (s, 1H), 7.80-7.78 (m, 1H), 7.53-7.50 (m, 2H), 7.25-7.22 (m, 2H), 6.18-5.89 (m, 3H), 4.84-4.00 (m, 5H), 3.08-2.65 (m, 5H), 1.61 (d, J=3.2 Hz, 9H), 1.29 (d, J=6.0 Hz, 3H).

Compound 39B

LC-MS (ESI): R_{T}=3.313 min, mass calcd. for C₂₉H₃₂Cl₂N₆O₄ 598.2, m/z found 599.2 [M+H]⁺. Chiral analysis (Column: Chiralpak IE; Column size: 5 μm 4.6 mm*250 mm; Mobile Phase: Hexane:IPA=80:20 at 30 mL/min; Col. Temp: 30° C.; Wave length: 254 nm; Rt=10.729 min). ¹H NMR (400 MHz, CDCl₃) δ 8.67 (s, 1H), 7.81-7.78 (m, 1H), 7.52-7.47 (m, 2H), 7.33-7.28 (m, 1H), 7.24-7.22 (m, 1H), 6.11-5.53 (m, 3H), 4.89-3.73 (m, 5H), 3.13-2.66 (m, 5H), 1.68-1.58 (m, 9H), 1.29 (d, J=6.0 Hz, 3H).

Compounds 40A and 40B

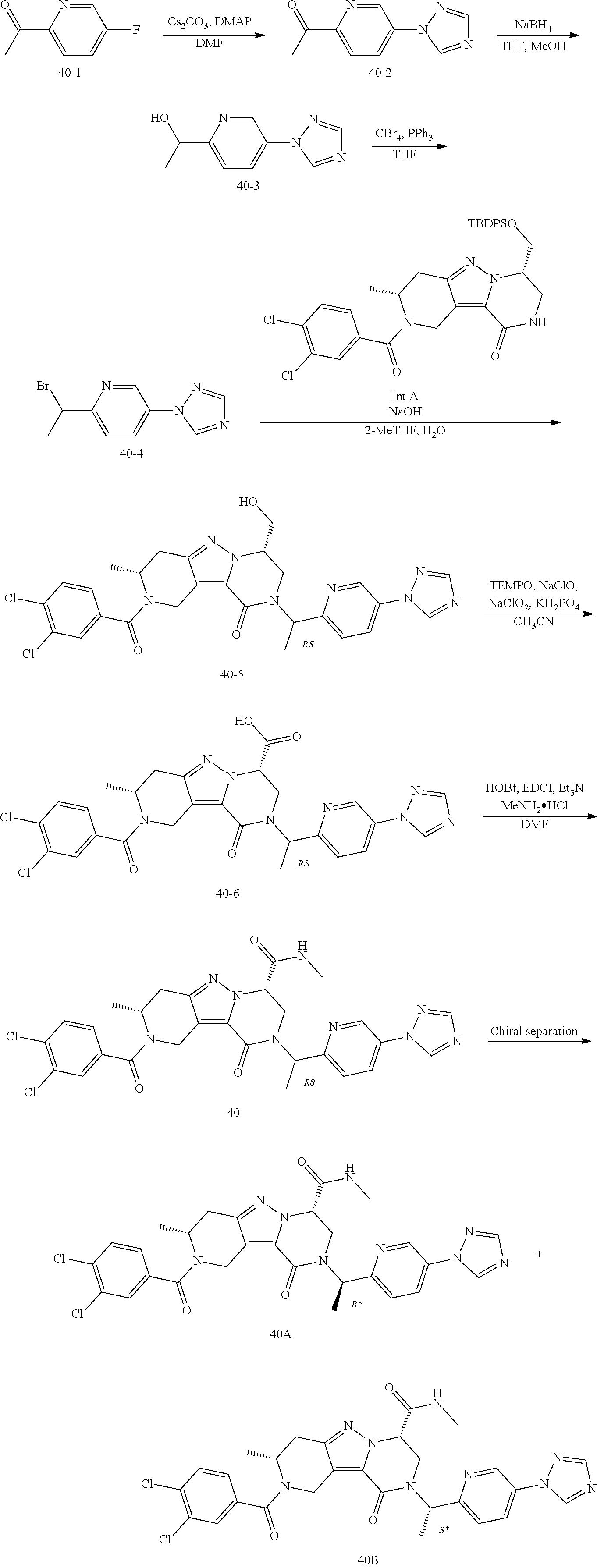

Intermediate 40-2

1-(5-(1H-1,2,4-Triazol-1-yl)pyridin-2-yl)ethan-1-one

To the solution of 1-(5-fluoropyridin-2-yl)ethan-1-one 40-1 (1.0 g, 7.19 mmol) and 1H-1,2,4-triazole (1.5 g, 21.7 mmol) in N,N-dimethylformamide (30 mL) was added cesium carbonate (4.7 g, 14.4 mmol) and N,N-dimethylpyridin-4-amine (0.9 g, 7.37 mmol) at room temperature. After being stirred at 80° C. for 6 hours, the mixture was diluted with water (100 mL) and extracted with ethyl acetate (200 mL) twice. The combined organic layers were washed with brine (100 mL), dried over Na₂SO₄₍ₛ₎ and filtered. The filtrate was concentrated and purified by C18 chromatography (acetonitrile:water (+0.02% ammonium bicarbonate)=30%-40%) to give the title product (900 mg, 100% purity from LCMS, 67% yield) as yellow solids. LC-MS (ESI): R_{T}=1.17 min, mass calcd. for C₉H₈N₄O 188.1, m/z found 189.1 [M+H]⁺.

Intermediate 40-3

1-(5-(1H-1,2,4-Triazol-1-yl)pyridin-2-yl)ethan-1-ol

To the solution of 1-(5-(1H-1,2,4-triazol-1-yl)pyridin-2-yl)ethan-1-one 40-2 (800 mg, 4.25 mmol) in tetrahydrofuran (24 mL) and methanol (8 mL) was added sodium tetrahydroborate (80 mg, 2.12 mmol) at 0° C. After being stirred at 0° C. for 1 hour, the mixture was added ethyl acetate (80 mL), washed with water (16 mL), dried over Na₂SO₄₍ₛ₎ and filtered. The filtrate was concentrated under reduced pressure to give crude title compound (800 mg, 100% purity from LCMS, 99% yield) as yellow oil. LC-MS (ESI): R_{T}=0.33 min, mass calcd. for C₉H₁₀N₄O 190.1, m/z found 191.1 [M+H]⁺.

Intermediate 40-4

2-(1-Bromoethyl)-5-(1H-1,2,4-triazol-1-yl)pyridine

To a solution of 1-(5-(1H-1,2,4-triazol-1-yl)pyridin-2-yl)ethan-1-ol 40-3 (800 mg, 100% purity, 4.21 mmol) in tetrahydrofuran (16 mL) was added perbromomethane (2.1 g, 6.33 mmol) and triphenylphosphine (2.2 g, 8.39 mmol) at 0° C. After being stirring for 1 hour at room temperature, the mixture was concentrated under reduced pressure and purified by C18 chromatography (acetonitrile:water (+0.02% ammonium bicarbonate)=45%-65%) to give the title product (450 mg, 78% purity, 33% yield) as white solids. LC-MS (ESI): R_{T}=1.39 min, mass calcd. for C₉H₉BrN₄ 252.0, m/z found 253.0 [M+H]⁺.

Intermediate 40-5

(3R,7S)-9-(1-(5-(1H-1,2,4-Triazol-1-yl)pyridin-2-yl)ethyl)-2-(3,4-dichlorobenzoyl)-7-(hydroxymethyl)-3-methyl-1,2,3,4,8,9-hexahydropyrido[4′,3′:3,4] pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a] pyrazin-10(7H)-one Int A (670 mg, 1.03 mmol) and 2-(1-bromoethyl)-5-(1H-1,2,4-triazol-1-yl)pyridine 40-4 (450 mg, 78% purity, 1.39 mmol) in 2-methyltetrahydrofuran (7 mL) was added 50% wt. sodium hydroxide in water (7 mL) slowly at 0° C. After being stirred at room temperature for 3 hours, the mixture was added ethyl acetate (100 mL) washed with water (100 mL) twice, brine (200 mL), dried over Na₂SO₄₍ₛ₎ and filtered. The filtrate was concentrated and purified by C18 chromatography (acetonitrile:water (+0.02% ammonium bicarbonate)=45%-65%) to give the title product (210 mg, 85% purity, 30% yield) as white solids. LC-MS (ESI): R_{T}=2.52 min, mass calcd. for C₂₇H₂₆Cl₂N₈O₃ 580.2, m/z found 581.0 [M+H]⁺.

Intermediate 40-6

(3R,7S)-9-(1-(5-(1H-1,2,4-Triazol-1-yl)pyridin-2-yl)ethyl)-2-(3,4-dichlorobenzoyl)-3-methyl-10-oxo-1,2,3,4,7,8,9,10-octahydropyrido[4′,3′:3,4]pyrazolo[1,5-a]pyrazine-7-carboxylic acid

To a solution of (3R,7S)-9-(1-(5-(1H-1,2,4-triazol-1-yl)pyridin-2-yl)ethyl)-2-(3,4-dichlorobenzoyl)-7-(hydroxymethyl)-3-methyl-1,2,3,4,8,9-hexahydropyrido[4′,3′:3,4]pyrazolo[1,5-a]pyrazin-10(7H)-one 40-5 (210 mg, 85% purity, 0.31 mmol) in acetonitrile (5 mL) were added 2,2,6,6-tetramethylpiperidinooxy (96 mg, 0.61 mmol), sodium hypochlorite aqueous solution (0.4 mL, 0.67 mmol), sodium chlorite (56 mg, 0.62 mmol), saturated sodium dihydrogenphosphate aqueous solution (5 mL) at 0° C. After being stirred at 0° C. for 6 hours, the mixture was diluted with water (20 mL) and extracted with ethyl acetate (40 mL) twice. The combined organic layers were washed with brine (40 mL), dried over Na₂SO₄₍ₛ₎ and filtered. The filtrate was concentrated to give the title compound (200 mg, 83% purity, 91% yield). LC-MS (ESI): R_{T}=1.27 min, mass calcd. for C₂₇H₂₄Cl₂N₈O₄ 594.1, m/z found 593.0 [M−H]⁻.

Compound 40

(3R,7S)-9-(1-(5-(1H-1,2,4-Triazol-1-yl)pyridin-2-yl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide

To a mixture of (3R,7S)-9-(1-(5-(1H-1,2,4-triazol-1-yl)pyridin-2-yl)ethyl)-2-(3,4-dichlorobenzoyl)-3-methyl-10-oxo-1,2,3,4,7,8,9,10-octahydropyrido[4′,3′:3,4] pyrazolo[1,5-a]pyrazine-7-carboxylic acid 40-6 (200 mg, 83% purity, 0.28 mmol), methanamine hydrochloride (47 mg, 0.70 mmol), 1-(3-dimethylaminopropyl)-3-ethylcarbodiimide hydrochloride (107 mg, 0.558 mmol) and 1H-benzo[d][1,2,3]triazol-1-ol (76 mg, 0.56 mmol) in N,N-dimethylformamide (5 mL) at 0° C. was added dropwise triethylamine (0.26 mL, 2.00 mmol) within 60 minutes. After being stirred at 0° C. for 1 hour, the mixture was diluted with water (30 mL), extracted with ethyl acetate (30 mL) twice. The combined organic layers were washed with brine (60 mL), dried over Na₂SO₄₍ₛ₎ and filtered. The filtrate was concentrated and purified by C18 column (acetonitrile:water (+0.02% ammonium bicarbonate)=45% to 60%) to give the title compound (140 mg, 100% purity from LCMS, 83% yield) as white solids. LC-MS (ESI): R_{T}=1.44 min, mass calcd. for C₂₈H₂₇Cl₂N₉O₃ 607.2, m/z found 608.6 [M+H]⁺.

Compounds 40A and 40B

(3R,7S)-9-((R*)-1-(5-(1H-1,2,4-Triazol-1-yl)pyridin-2-yl)ethyl)-2-(3,4-dichlorobenzoyl)-N,3-dimethyl-10-oxo-1,2,3,4,7,8,9,10-octahydropyrido[4′,3′:3,4]pyrazolo [1,5-a]pyrazine-7-carboxamide (40A), and (3R,7S)-9-((S*)-1-(5-(1H-1,2,4-triazol-1-yl)pyridin-2-yl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide (40B)

A racemic mixture of (3R,7S)-9-(1-(5-(1H-1,2,4-triazol-1-yl)pyridin-2-yl)ethyl)-2-(3,4-dichlorobenzoyl)-N,3-dimethyl-10-oxo-1,2,3,4,7,8,9,10-octahydropyrido[4′,3′:3,4]pyrazolo[1,5-a]pyrazine-7-carboxamide 40 (160 mg, 100% purity, 0.26 mmol) was separated by chiral Prep. HPLC (separation condition: Column: Chiralpak IB N-5 5 μm 20*250 mm; Mobile Phase: ACN:IPA=90:10 at 15 mL/min; Temp: 30° C.; Wavelength: 254 nm) to afford Peak 1 (50 mg, 87.8% purity from SFC, 27% yield) and compound 40B (65 mg, 99.7% purity, 41% yield, 98.9% stereopure) as white solids. Peak 1 was separated by chiral Prep. HPLC (separation condition: Column: Chiralpak IE 5 μm 20*250 mm; Mobile Phase: MeOH:DCM=50:50 at 15 mL/min; Temp: 30° C.; Wavelength: 230 nm) to afford the title compound 40A (35 mg, 99.6% purity, 79% yield, 100% stereopure) as white solids.

Compound 40A

LC-MS (ESI): R_{T}=2.977 min, mass calcd. for C₂₈H₂₇Cl₂N₉O₃ 607.2, m/z found 608.2 [M+H]⁺. Chiral analysis (Column: Chiralpak IB N-5 5 μm 4.6*250 mm; Mobile Phase: ACN:IPA=90:10 at 1 mL/min; Temp: 30° C.; Wavelength: 254 nm; Rt=5.774 min). ¹H NMR (400 MHz, CDCl₃) δ 8.93 (d, J=2.4 Hz, 1H), 8.61 (s, 1H), 8.15 (s, 1H), 8.00-7.98 (m, 1H), 7.54-7.51 (m, 2H), 7.43 (d, J=8.4 Hz, 1H), 7.27-7.25 (m, 1H), 5.98-5.35 (m, 3H), 4.89-4.16 (m, 4H), 4.04-4.00 (m, 1H), 3.06 (s, 1H), 2.73-2.69 (m, 1H), 2.66 (d, J=4.8 Hz, 3H), 1.69 (d, J=6.8 Hz, 3H), 1.29 (d, J=6.8 Hz, 3H).

Compound 40B

LC-MS (ESI): R_{T}=2.972 min, mass calcd. for C₂₈H₂₇Cl₂N₉O₃ 607.2, m/z found 608.2 [M+H]⁺. Chiral analysis (Column: Chiralpak IB N-5 5 μm 4.6*250 mm; Mobile Phase: ACN:IPA=90:10 at 1 mL/min; Temp: 30° C.; Wavelength: 254 nm; Rt=9.858 min). ¹H NMR (400 MHz, CDCl₃) δ 8.91 (d, J=2.4 Hz, 1H), 8.58 (s, 1H), 8.15 (s, 1H), 8.01-7.99 (m, 1H), 7.51-7.49 (m, 3H), 7.26-7.23 (m, 1H), 5.94-5.37 (m, 3H), 4.91-4.41 (m, 4H), 3.87-3.82 (m, 1H), 3.05-3.02 (m, 1H), 2.82 (d, J=4.8 Hz, 3H), 2.72 (d, J=16.4 Hz, 1H), 1.68 (d, J=7.2 Hz, 3H), 1.29 (d, J=6.8 Hz, 3H).

Compounds 41A and 41B

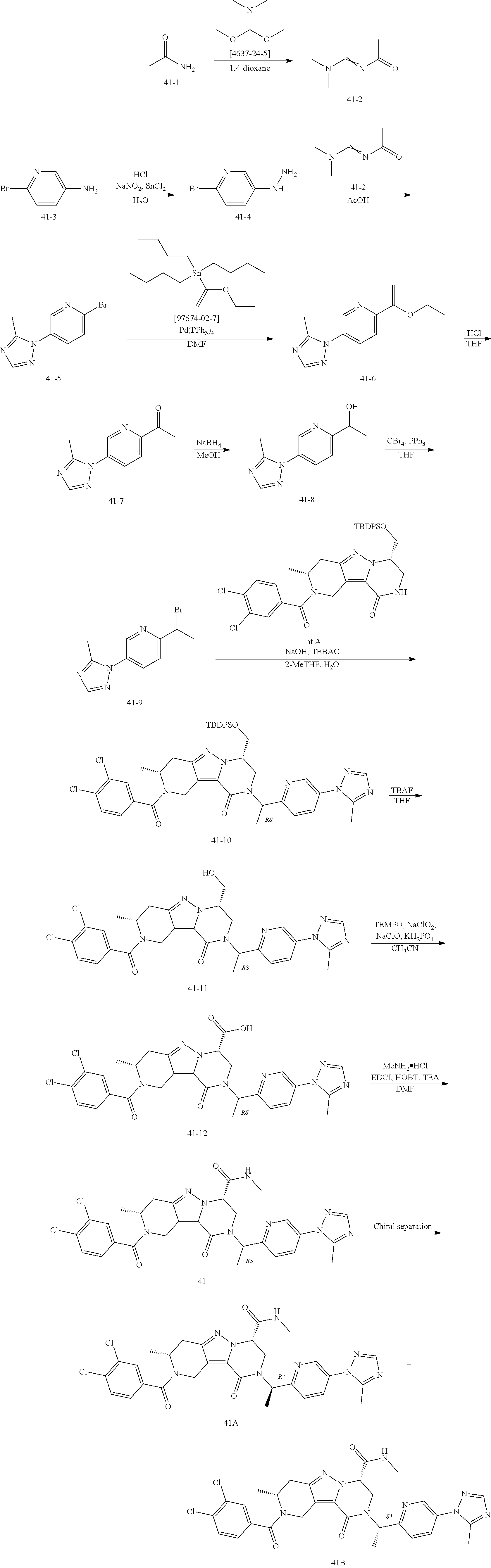

Intermediate 41-2

N-((Dimethylamino)methylene)acetamide

To a solution of acetamide 41-1 (1.0 g, 16.9 mmol) in 1,4-dioxane (35 mL) was added 1,1-dimethoxy-N,N-dimethylmethanamine (3.4 ml, 25.59 mmol) at 0° C. Then the reaction mixture was stirred at 80° C. under nitrogen atmosphere for 2 hours. The reaction mixture was concentrated to give the title compound (2.0 g, 96% purity from LCMS, 99% yield) as yellow oil. LC-MS (ESI): R_{T}=0.234 min, mass calcd. for C₅H₁₀N₂O 114.1, m/z found 115.1 [M+H]⁺.

Intermediate 41-4

2-Bromo-5-hydrazinylpyridine

A solution of 6-bromopyridin-3-amine 41-3 (8.00 g, 90% purity, 41.6 mmol) in 6 M hydrochloric acid aqueous solution (70 mL, 420 mmol) was cooled to 0° C. A solution of sodium nitrite (2.90 g, 42.0 mmol) in water (120 mL) precooled to 0° C. was added over 5 minutes and the reaction mixture was stirred for 45 minutes at 0° C. Stannous chloride dihydrate (24.0 g, 106 mmol) was suspended in 6 M aqueous hydrochloric acid aqueous solution (70 mL, 420 mmol) precooled to 0° C. Then, it was added to the reaction mixture over 5 minutes. The reaction mixture was stirred for 60 minutes at 0° C. The reaction was quenched via addition of 40% w/w solution of potassium hydroxide in water until the PH is 12-14. The mixture was diluted with water and ethyl acetate. The organic layers were separated, and the aqueous layer was extracted with ethyl acetate (200 mL) three times. The combined organic layers were dried over sodium sulfate, filtered and concentrated to give the desired compound (8.00 g, 65% purity from LCMS, 67% yield) as brown solids. LC-MS (ESI): R_{T}=1.003 min, mass calcd. for C₅H₆BrN₃ 187.0, m/z found 188.0 [M+H]⁺.

Intermediate 41-5

2-Bromo-5-(5-methyl-1H-1,2,4-triazol-1-yl)pyridine

The 2-bromo-5-hydrazinylpyridine 41-4 (2.40 g, 82% purity, 10.5 mmol) in acetic acid (40 mL) was added N-((dimethylamino)methylene)acetamide 41-2 (3.00 g, 96% purity, 25.1 mmol) at room temperature. After being heated at 90° C. for 2 hours, the mixture was filtered and the filtrate was extracted with ethyl acetate (100 mL) three times. The combined organic layers were washed with water (50 mL), brine (50 mL) and concentrated to get the desired compound (2.10 g, 56% purity from LCMS, 47% yield) as yellow solids. LC-MS (ESI): R_{T}=1.24 min, mass calcd. for C₈H₇BrN₄ 238.0, m/z found 239.0 [M+H]⁺.

Intermediate 41-6

2-(1-Ethoxyvinyl)-5-(5-methyl-11H-1,2,4-triazol-1-yl)pyridine

To a solution of 2-bromo-5-(5-methyl-1H-1,2,4-triazol-1-yl)pyridine 41-5 (8.20 g, 50% purity, 17.2 mmol) and tributyl(1-ethoxyvinyl)stannane (20 mL, 59.2 mmol) in N,N-dimethylformamide (160 mL) was added tetrakis(triphenylphosphine)palladium (2.00 g, 1.73 mmol) under nitrogen atmosphere. After being heated at 100° C. for 2 hours, the mixture was diluted with saturated potassium fluoride aqueous solution (200 mL) and filtered. The filtrate was extracted with ethyl acetate (200 mL) three times. The combined organic layers were washed with brine (80 mL), dried over Na₂SO₄₍ₛ₎ and filtered. The filtrate was concentrated and purified by C18 chromatography (acetonitrile:water (+0.02% ammonium bicarbonate)=50% to 70%) to give the desired compound (1.90 g, 92% purity from LCMS, 45% yield) as yellow solids. LC-MS (ESI): R_{T}=1.294 min, mass calcd. for C₁₂H₁₄N₄O 230.1, m/z found 231.2 [M+H]⁺.

Intermediate 41-7

1-(5-(5-Methyl-1H-1,2,4-triazol-1-yl)pyridin-2-yl)ethanone

To a solution of 2-(1-ethoxyvinyl)-5-(5-methyl-1H-1,2,4-triazol-1-yl)pyridine 41-6 (2.00 g, 100% purity, 8.69 mmol) in tetrahydrofuran (20 mL) was added 2 M hydrogen chloride in water (20 mL, 40 mmol) at 0° C. under nitrogen atmosphere. After being stirred at room temperature for 2 hours, the reaction mixture was added saturated sodium bicarbonate aqueous solution (50 mL) and extracted with ethyl acetate (100 mL) for three times. The combined organic layers were washed with brine (50 mL), dried Na₂SO₄₍ₛ₎, and concentrated to give the title compound (1.80 g, 90% purity from ¹H NMR, 92% yield) as yellow solids. LC-MS (ESI): R_{T}=1.055 min, mass calcd. for C₁₀H₁₀N₄O 202.1, m/z found 203.1 [M+H]⁺. ¹H NMR (300 MHz, CDCl₃) δ 8.91 (d, J=2.4 Hz, 1H), 8.26 (d, J=8.4 Hz, 1H), 8.05-8.02 (m, 2H), 2.81 (s, 3H), 2.69 (s, 3H).

Intermediate 41-8

1-(5-(5-Methyl-1H-1,2,4-triazol-1-yl)pyridin-2-yl)ethanol

To the solution of 1-(5-(5-methyl-1H-1,2,4-triazol-1-yl)pyridin-2-yl)ethanone 41-7 (3.00 g, 90% purity, 13.4 mmol) in methanol (40 mL) was added sodium borohydride (500 mg, 13.2 mmol) at 0° C. After being stirred at 0° C. for 1 hour, the mixture was quenched with saturated ammonium chloride aqueous solution (50 mL), extracted with ethyl acetate (100 mL) three times, dried over Na₂SO₄₍ₛ₎ and filtered. The filtrate was concentrated and purified by silica gel column chromatography (petroleum ether:ethyl acetate=0:1) to give the title compound (2.00 g, 90% purity from ¹H NMR, 66% yield) as yellow oil. ¹H NMR (300 MHz, CDCl₃) δ 8.71 (d, J=2.1 Hz, 1H), 8.00 (s, 1H), 7.89-7.85 (m, 1H), 7.55 (d, J=8.4 Hz, 1H), 5.07-5.00 (m, 1H), 4.16-3.94 (m, 1H), 2.60 (s, 3H), 1.59 (d, J=6.6 Hz, 3H).

Intermediate 41-9

2-(1-Bromoethyl)-5-(5-methyl-1H-1,2,4-triazol-1-yl)pyridine

To a solution of 1-(5-(5-methyl-1H-1,2,4-triazol-1-yl)pyridin-2-yl)ethanol 41-8 (1.20 g, 90% purity, 5.29 mmol) in tetrahydrofuran (20 mL) was added triphenylphosphine (2.70 g, 10.3 mmol) and perbromomethane (3.00 g, 9.05 mmol) at 0° C. After being stirred at 25° C. for 2 hours, the reaction was concentrated to get a residue, which was purified by column chromatography on silica gel (petroleum ether:ethyl acetate=10:1 to 0:1) to give the desired compound (1.00 g, 90% purity from ¹H NMR, 64% yield) as white solid. ¹H NMR (300 MHz, CDCl₃) δ 8.74 (d, J=2.4 Hz, 1H), 8.01 (s, 1H), 7.90-7.86 (m, 1H), 7.51-7.46 (m, 1H), 5.32 (q, J=13.8 Hz, 1H), 2.63 (s, 3H), 2.14 (d, J=6.9 Hz, 3H).

Intermediate 41-10

(3R,7S)-7-(((tert-Butyldiphenylsilyl)oxy)methyl)-2-(3,4-dichlorobenzoyl)-3-methyl-9-(1-(5-(5-methyl-1H-1,2,4-triazol-1-yl)pyridin-2-yl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (900 mg, 90% purity, 1.25 mmol), benzyltriethylammonium chloride (135 mg, 0.593 mmol) and 2-(1-bromoethyl)-5-(5-methyl-1H-1,2,4-triazol-1-yl)pyridine 41-9 (700 mg, 90% purity, 2.36 mmol) in 2-methyltetrahydrofuran (9 mL) was added 50% wt. sodium hydroxide in water (9 mL) slowly at 30° C. After being stirred at 30° C. for 4 hours, the reaction mixture was diluted with water (50 mL) and extracted with ethyl acetate (100 mL) twice. The combined organic layers were washed with brine (50 mL) twice, dried over Na₂SO₄₍ₛ₎, filtered and concentrated under reduced pressure to give the title compound (1.50 g, 33% purity from LCMS, 47% yield) as yellow solids. LC-MS (ESI): R_{T}=2.08 min and 2.17 min, mass calcd. for C₄₄H₄₆Cl₂N₈O₃Si 832.3, m/z found 833.3 [M+H]⁺.

Intermediate 41-11

(3R,7S)-2-(3,4-Dichlorobenzoyl)-7-(hydroxymethyl)-3-methyl-9-(1-(5-(5-methyl-1H-1,2,4-triazol-1-yl)pyridin-2-yl)ethyl)-1,2,3,4,8,9-hexahydropyrido[4′,3′:3,4]pyrazolo [1,5-a]pyrazin-10(7H)-one

To a solution of (3R,7S)-7-(((tert-butyldiphenylsilyl)oxy)methyl)-2-(3,4-dichlorobenzo yl)-3-methyl-9-(1-(5-(5-methyl-1H-1,2,4-triazol-1-yl)pyridin-2-yl)ethyl)-1,2,3,4,8,9-hexa hydropyrido[4′,3′:3,4]pyrazolo[1,5-a]pyrazin-10(7H)-one 41-10 (2.00 g, 30% purity, 0.720 mmol) in tetrahydrofuran (15 mL) was added 1 M tetrabutylammonium fluoride (2 mL, 2 mmol) at 0° C. After being stirred at room temperature for 3 hours under nitrogen atmosphere, the mixture was diluted with water (50 mL) and extracted with ethyl acetate (80 mL) twice. The combined organic layers were washed with brine (50 mL), dried over Na₂SO₄₍ₛ₎ and filtered. The filtrate was concentrated and purified by C18 column (acetonitrile:water (0.1% ammonium bicarbonate)=5%-95%) to give the title compound (480 mg, 88% purity from LCMS, 99% yield) as yellow solids. LC-MS (ESI): R_{T}=1.47 min, mass calcd. for C₂₈H₂₈Cl₂N₈O₃ 594.2, m/z found 595.1 [M+H]⁺.

Intermediate 41-12

(3R,7S)-2-(3,4-Dichlorobenzoyl)-3-methyl-9-(1-(5-(5-methyl-1H-1,2,4-triazol-1-yl)pyridin-2-yl)ethyl)-10-oxo-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-7-(hydroxymethyl)-3-methyl-9-(1-(5-(5-methyl-1H-1,2,4-triazol-1-yl)pyridin-2-yl)ethyl)-1,2,3,4,8,9-hexahydropyrido[4′,3′:3,4]pyrazolo[1,5-a]pyrazin-10(7H)-one 41-11 (380 mg, 88% purity, 0.561 mmol) in acetonitrile (5 mL) was added saturated potassium dihydrogenphosphate aqueous solution (5 mL), sodium chlorite (120 mg, 80% purity, 1.06 mmol), 2,2,6,6-tetramethylpiperidinooxy (200 mg, 1.28 mmol) and sodium hypochlorite aqueous solution (0.8 mL, 10% purity, 1.25 mmol) at 0° C.

After being stirred at 20° C. for 4 hours, the reaction mixture was purified by C18 column (acetonitrile:water (0.1% ammonium bicarbonate)=5%-95%) to give the title compound (350 mg, 95% purity from LCMS, 98% yield) as white solids. LC-MS (ESI): R_{T}=1.25 min, mass calcd. for C₂₈H₂₆Cl₂N₈O₄ 608.1, m/z found 609.1 [M+H]⁺.

Compound 41

(3R,7S)-2-(3,4-Dichlorobenzoyl)-N,3-dimethyl-9-(1-(5-(5-methyl-1H-1,2,4-triazol-1-yl)pyridin-2-yl)ethyl)-10-oxo-1,2,3,4,7,8,9,10-octahydropyrido[4′,3′:3,4]pyrazolo[1,5-a]pyrazine-7-carboxamide

To a solution of (3R,7S)-2-(3,4-dichlorobenzoyl)-3-methyl-9-(1-(5-(5-methyl-1H-1,2,4-triazol-1-yl)pyridin-2-yl)ethyl)-10-oxo-1,2,3,4,7,8,9,10-octahydropyrido[4′,3′:3,4]pyrazolo[1,5-a]pyrazine-7-carboxylic acid 41-12 (340 mg, 90% purity, 0.502 mmol), 1H-benzo[d][1,2,3]triazol-1-ol (130 mg, 0.962 mmol), 1-(3-dimethylamino propyl)-3-ethylcarbodiimide hydrochloride (190 mg, 0.991 mmol) and methanamine hydrochloride (100 mg, 1.48) in N,N-dimethylformamide (6 mL) was added triethylamine (320 mg, 3.16 mol) at 0° C. After being stirred at room temperature for 4 hours, the reaction mixture was directly purified by C18 column (acetonitrile:water (0.1% ammonium bicarbonate)=5%-95%) to give the title compound (200 mg, 91% purity from LCMS, 58% yield) as white solids. LC-MS (ESI): R_{T}=1.43 min, mass calcd. for C₂₉H₂₉Cl₂N₉O₃ 621.2, m/z found 622.1 [M+H]⁺.

Compounds 41A and 41B

(3R,7S)-2-(3,4-Dichlorobenzoyl)-N,3-dimethyl-9-((R*)-1-(5-(5-methyl-1H-1,2,4-triazol-1-yl)pyridin-2-yl)ethyl)-10-oxo-1,2,3,4,7,8,9,10-octahydropyrido[4′,3′:3,4] pyrazolo[1,5-a]pyrazine-7-carboxamide (41A), and (3R,7S)-2-(3,4-dichlorobenzoyl)-N,3-dimethyl-9-((S*)-1-(5-(5-methyl-1H-1,2,4-triazol-1-yl)pyridin-2-yl)ethyl)-10-oxo-1,2,3,4,7,8,9,10-octahydropyrido[4′,3′:3,4] pyrazolo[1,5-a]pyrazine-7-carboxamide (41B)

(3R,7S)-2-(3,4-dichlorobenzoyl)-N,3-dimethyl-9-(1-(5-(5-methyl-1H-1,2,4-triazol-1-yl)pyridin-2-yl)ethyl)-10-oxo-1,2,3,4,7,8,9,10-octahydropyrido[4′,3′:3,4] pyrazolo[1,5-a]pyrazine-7-carboxamide 41 (200 mg, 91% purity, 0.291 mmol) was separated by Chiral Prep-HPLC (Column: Chiralpak IC 10 μm 30 mm*250 mm; Mobile Phase: ACN:IPA=70:30 at 25 mL/min; Col. Temp: 30° C.; Wavelength: 254 nm) to afford the title compound 41A (40 mg, 99.8% purity from LCMS, 22.0% yield, 100% stereopure) as a white solid and compound 41B (50 mg, 99.8% purity from LCMS, 27.5% yield, 100% stereopure) as a white solid.

Compound 41A

LC-MS (ESI): R_{T}=3.237 min, mass calcd. for C₂₉H₂₉Cl₂N₉O₃ 621.2, m/z found 622.2 [M+H]⁺. Chiral analysis (Column: Chiralpak IC 5 μm, 4.6*250 mm; Mobile Phase: ACN:IPA=70:30 at 1 mL/min; Temp 30° C.; Wavelength: 254 nm; R_{T}=6.179 min). ¹H NMR (400 MHz, CDCl₃) δ 8.71 (d, J=2.4 Hz, 1H), 7.98 (s, 1H), 7.80-7.77 (m, 1H), 7.54-7.43 (m, 3H), 7.27-7.25 (m, 1H), 6.02-4.49 (m, 6H), 4.22-4.16 (m, 1H), 4.03-3.98 (m, 1H), 3.14-2.98 (m, 1H), 2.75-2.69 (m, 1H), 2.64 (d, J=4.8 Hz, 3H), 2.61 (s, 3H), 1.70 (d, J=6.8 Hz, 3H), 1.29 (d, J=6.4 Hz, 3H).

Compound 41B

LC-MS (ESI): R_{T}=3.178 min, mass calcd. for C₂₉H₂₉Cl₂N₉O₃ 621.2, m/z found 622.2 [M+H]⁺. Chiral analysis (Column: Chiralpak IC 5 μm, 4.6*250 mm; Mobile Phase: ACN:IPA=70:30 at 1 mL/min; Temp 30° C.; Wavelength: 254 nm; R_{T}=7.120 min). ¹H NMR (400 MHz, CDCl₃) δ 8.69 (d, J=2.4 Hz, 1H), 7.97 (s, 1H), 7.83-7.80 (m, 1H), 7.52-7.50 (m, 3H), 7.26-7.23 (m, 1H), 5.95-4.45 (m, 7H), 3.89-3.84 (m, 1H), 3.13-2.70 (m, 5H), 2.57 (s, 3H), 1.69 (d, J=7.2 Hz, 3H), 1.30 (d, J=6.4 Hz, 3H).

Compounds 42A and 42B

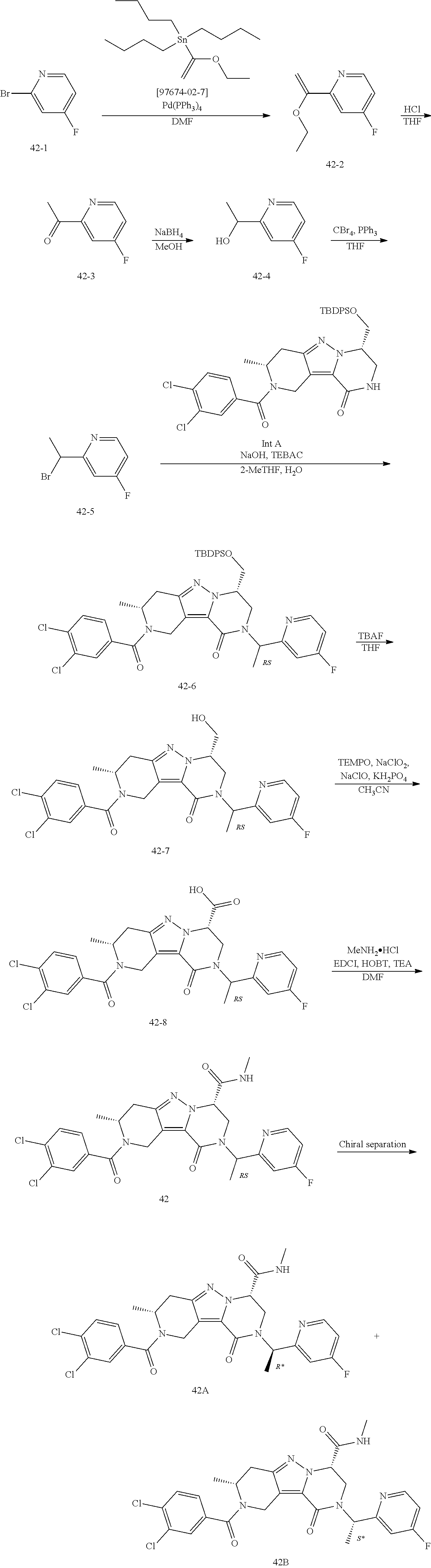

Intermediate 42-2

2-(1-Ethoxyvinyl)-4-fluoropyridine

A mixture of 2-bromo-4-fluoropyridine 42-1 (2 g, 11.4 mmol), tributyl(1-ethoxyvinyl)stannane (7 ml, 20.7 mmol) and tetrakis(triphenylphosphine)palladium(0) (300 mg, 0.26 mmol) in N,N-dimethylformide (20 ml) was stirred at 120° C. under nitrogen atmosphere for 8 hours. After being cooled down to room temperature, the reaction mixture was quenched with saturated potassium fluoride aqueous solution (30 mL). The mixture was stirred at room temperature for 1.5 hours, and extracted with ethyl acetate (80 mL) for three times. The combined organic layers were washed with brine (20 mL), dried over Na₂SO₄₍ₛ₎, and filtered. The filtrate was concentrated and purified by silica gel column chromatography (petroleum ether:ethyl acetate=100:1 to 10:1) to give the title compound (1.52 g, 99% purity from LCMS, 79% yield) as yellow oil. LC-MS (ESI): R_{T}=1.626 min, mass calcd. for C₉H₁₀FNO 167.1, m/z found 168.2 [M+H]⁺.

Intermediate 42-3

1-(4-Fluoropyridin-2-yl)ethanone

To a solution of 2-(1-ethoxyvinyl)-4-fluoropyridine 42-2 (1.52 g, 99% purity, 9.00 mmol) in tetrahydrofuran (28 mL) was added 1 M hydrochloride aqueous solution in water (14 mL) at 0° C. After being stirred at room temperature for 5 hours, the reaction mixture was acidized with saturated sodium bicarbonate aqueous solution to pH=8˜9, and extracted with dichloromethane (50 mL) three times. The combined organic layers were washed with brine (20 mL) twice, dried over Na₂SO₄₍ₛ₎ and filtered. The filtrate was concentrated and purified by column chromatography on silica gel (petroleum ether:acetone=100:1-50:1) to give the title compound (1.1 g, 71% purity from LCMS, 62% yield) as yellow oil. LC-MS (ESI): R_{T}=1.139 min, mass calcd. for C₇H₆FNO 139.0, m/z found 140.2 [M+H]⁺.

Intermediate 42-4

1-(4-Fluoropyridin-2-yl)ethanol

To a solution of 1-(4-fluoropyridin-2-yl)ethanone 42-3 (1.1 g, 71% purity, 5.61 mmol) in methanol (12 mL) was slowly added sodium tetrahydroborate (215 mg, 5.68 mmol) at 0° C. After being stirred at room temperature for 2 hours, the reaction was quenched with acetone (10 mL) dropwise and concentrated to give a residue, which was purified by column chromatography on silica gel (dichloromethane:methanol=100:1-50:1) to give the title compound (365 mg, 99% purity from LCMS, 46% yield) as colorless oil. LC-MS (ESI): R_{T}=0.501 min, mass calcd. for C₇H₈FNO 141.1, m/z found 142.1 [M+H]⁺. ¹H NMR (400 MHz, DMSO-d₆) δ 8.53-8.50 (m, 1H), 7.33-7.30 (m, 1H), 7.19-7.14 (m, 1H), 5.51-5.50 (m, 1H), 4.77-4.71 (m, 1H), 1.37 (d, J=6.4 Hz, 3H).

Intermediate 42-5

2-(1-Bromoethyl)-4-fluoropyridine

To a solution of 1-(4-fluoropyridin-2-yl)ethanol 42-4 (565 mg, 95% purity, 3.80 mmol) in tetrahydrofuran (25 mL) was added triphenylphosphine (1.8 g, 6.86 mmol) and perbromomethane (1.8 g, 5.43 mmol) at 0° C. After being stirred at room temperature for 2 hours, the mixture was filtered. The filtrate was concentrated and purified by column chromatography on silica gel (petroleum ether:ethyl acetate=100:1-60:1) to give the title compound (600 mg, 97% purity from LCMS, 75% yield) as colorless oil. LC-MS (ESI): R_{T}=1.318 min, mass calcd. for C₇H₇BrFN 203.0, m/z found 204.0 [M+H]⁺. ¹H NMR (400 MHz, DMSO-d₆) δ 8.63-8.59 (m, 1H), 7.54-7.51 (m, 1H), 7.31-7.27 (m, 1H), 5.46 (q, J=7.2 Hz, 1H), 2.00 (d, J=6.8 Hz, 3H).

Intermediate 42-6

(3R,7S)-7-(((tert-Butyldiphenylsilyl)oxy)methyl)-2-(3,4-dichlorobenzoyl)-9-(1-(4-fluoropyridin-2-yl)ethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (1 g, 90% purity, 1.39 mmol), 2-(1-bromoethyl)-4-fluoropyridine 42-5 (400 mg, 97% purity, 1.90 mmol) and benzyltriethylammonium chloride (65 mg, 0.29 mmol) in 2-methyltetrahydrofuran (9 mL) and 50% wt. sodium hydroxide aqueous solution (9 mL). The reaction mixture was stirred at room temperature under nitrogen atmosphere for 3 hours, then quenched with water (10 mL) and extracted with ethyl acetate (25 mL) twice. The combined organic layers were dried over Na₂SO₄₍ₛ₎ and filtered. The filtrate was concentrated under reduced pressure to give crude, which was purified by C18 column (acetonitrile:water=5% to 90%) to give the title compound (1.15 g, 86% purity from LCMS, 92% yield) as white solids. LC-MS (ESI): R_{T}=1.680 min, mass calcd. for C₄₁H₄₂Cl₂FN₅O₃Si 770.8, m/z found 771.0 [M+H]⁺.

Intermediate 42-7

(3R,7S)-2-(3,4-Dichlorobenzoyl)-9-(1-(4-fluoropyridin-2-yl)ethyl)-7-(hydroxymethyl)-3-methyl-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(3,4-dichlorobenzoyl)-9-(1-(4-fluoropyridin-2-yl)ethyl)-3-methyl-1,2,3,4,8,9-hexahydropyrido[4′,3′:3,4]pyrazolo[1,5-a]pyrazin-10(7H)-one 42-6 (1.15 g, 86% purity, 1.28 mmol) in tetrahydrofuran (12 mL) was added 1 M tetrabutylammonium fluoride in tetrahydrofuran (1.2 mL, 1.2 mmol) at 0° C., then the mixture was stirred at room temperature for 2 hours. The reaction mixture was poured into ethyl acetate (25 mL) and washed with water (10 mL) three times. The combined organic layers were dried over Na₂SO₄₍ₛ₎ and filtered. The filtrate was concentrated under reduced pressure to give the residue, which was purified by silica gel column chromatography (dichloromethane:methanol=90:1-50:1) to give the title compound (680 mg, 99% purity from LCMS, 99% yield) as yellow solids. LC-MS (ESI): R_{T}=1.457 min, mass calcd. for C₂₅H₂₄Cl₂FN₅O₃ 531.1, m/z found 532.2 [M+H]⁺.

Intermediate 42-8

(3R,7S)-2-(3,4-Dichlorobenzoyl)-9-(1-(4-fluoropyridin-2-yl)ethyl)-3-methyl-10-oxo-1,2,3,4,7,8,9,10-octahydropyrido[4′,3′:3,4]pyrazolo[1,5-a]pyrazine-7-carboxylic acid

To a solution of (3R,7S)-2-(3,4-dichlorobenzoyl)-9-(1-(4-fluoropyridin-2-yl)ethyl)-7-(hydroxymethyl)-3-methyl-1,2,3,4,8,9-hexahydropyrido[4′,3′:3,4]pyrazolo[1,5-a]pyrazin-10(7H)-one 42-7 (680 mg, 99% purity, 1.26 mmol) in acetonitrile (7 mL) were added saturated potassium dihydrogen phosphate aqueous solution (7 mL), 2,2,6,6-tetramethylpiperidinooxy (403 mg, 2.58 mmol), sodium chlorite (295 mg, 2.61 mmol), sodium hypochlorite aqueous solution aqueous solution (1.55 mL, 2.60 mmol) at 0° C. The reaction was allowed to slowly return to room temperature. After being stirred at room temperature overnight, the reaction mixture was quenched with saturated sodium thiosulfate (15 mL), acidized with 1 M hydrochloride aqueous solution to pH=6˜7, and extracted with ethyl acetate (30 mL) for three times. The combined organic layers were washed with brine (10 mL), dried over Na₂SO₄₍ₛ₎, filtered. The filtrate was concentrated under reduced pressure to give crude, which was purified by C18 column (acetonitrile:water=5% to 90%) to give the title compound (580 mg, 99% purity from LCMS, 83% yield) as white solids. LC-MS (ESI): R_{T}=1.404 min, mass calcd. for C₂₅H₂₂Cl₂FN₅O₄ 545.1, m/z found 546.2 [M+H]⁺.

Compound 42

(3R,7S)-2-(3,4-Dichlorobenzoyl)-9-(1-(4-fluoropyridin-2-yl)ethyl)-N,3-dimethyl-10-oxo-1,2,3,4,7,8,9,10-octahydropyrido[4′,3′:3,4]pyrazolo[1,5-a]pyrazine-7-carboxamide

To a solution of (3R,7S)-2-(3,4-dichlorobenzoyl)-9-(1-(4-fluoropyridin-2-yl)ethyl)-3-methyl-10-oxo-1,2,3,4,7,8,9,10-octahydropyrido[4′,3′:3,4]pyrazolo[1,5-a]pyrazine-7-carboxylic acid 42-8 (580 mg, 99% purity, 1.05 mmol), methylamine hydrochloride (184 mg, 2.73 mmol), 1-(3-dimethylaminopropyl)-3-ethylcarbodiimide hydrochloride (418 mg, 2.18 mmol) and 1H-benzo[d][1,2,3]triazol-1-ol (298 mg, 2.21 mmol) in N,N-dimethylformamide (10 mL) was added triethylamine (1 mL, 7.21 mmol) at 0° C. The reaction was allowed to slowly return to room temperature. After being stirred at room temperature under nitrogen atmosphere for 3 hours, the reaction mixture was quenched with water (10 mL) and extracted with ethyl acetate (25 mL) three times. The combined organic layers were dried over Na₂SO₄₍ₛ₎ and filtered. The filtrate was concentrated and purified by C18 column (acetonitrile:water=5% to 100%) to give the title compound (370 mg, 100% purity from LCMS, 63% yield) as white solids. LC-MS (ESI): R_{T}=1.52 min, mass calcd. for C₂₆H₂₅Cl₂FN₆O₃ 558.1, m/z found 559.4 [M+H]⁺.

Compounds 42A and 42B

(3R,7S)-2-(3,4-Dichlorobenzoyl)-9-((R*)-1-(4-fluoropyridin-2-yl)ethyl)-N,3-dimethyl-10-oxo-1,2,3,4,7,8,9,10-octahydropyrido[4′,3′:3,4]pyrazolo[1,5-a]pyrazine-7-carboxamide (42A), and (3R,7S)-2-(3,4-dichlorobenzoyl)-9-((S*)-1-(4-fluoropyridin-2-yl)ethyl)-N,3-dimethyl-10-oxo-1,2,3,4,7,8,9,10-octahydropyrido[4′,3′:3,4]pyrazolo[1,5-a]pyrazine-7-carboxamide (42B)

A mixture of (3R,7S)-2-(3,4-dichlorobenzoyl)-9-(1-(4-fluoropyridin-2-yl)ethyl)-N,3-dimethyl-10-oxo-1,2,3,4,7,8,9,10-octahydropyrido[4′,3′:3,4]pyrazolo[1,5-a]pyrazine-7-carboxamide 42 (350 mg, 100% purity, 0.626 mmol) was separated by chiral. HPLC (separation condition: Column: Chiralpak IE 5 μm 20*250 mm; Mobile Phase: Hex:EtOH=30:70 at 60 g/min; Temp: 30° C.; Wavelength: 254 nm.) to afford the title compound 42A (79.5 mg, 22.2% yield, 97.7% purity, 100% stereopure) as a white solid and compound 42B (80.9 mg, 23% yield, 99.7% purity, 100% stereopure) as a white solid.

Compound 42A

LC-MS (ESI): R_{T}=3.568 min, mass calcd. for C₂₆H₂₅Cl₂FN₆O₃ 558.1, m/z found 559.2 [M+H]⁺. Chiral analysis (Column: Chiralpak IE 5 μm 4.6*250 mm; Mobile Phase: Hex:EtOH=30:70 at 1 mL/min; Col. Temp: 30° C.; Wavelength: 254 nm; R_{T}=7.629 min). ¹H NMR (400 MHz, DMSO-d₆) δ 8.60-8.56 (m, 1H), 7.88 (d, J=4.4 Hz, 1H), 7.76-7.74 (m, 2H), 7.46 (d, J=8.0 Hz, 1H), 7.29-7.26 (m, 1H), 7.08 (br s, 1H), 5.87-5.65 (m, 1H), 5.43-5.24 (m, 1H), 5.02 (s, 1H), 4.61-4.42 (m, 1H), 4.26-4.00 (m, 2H), 3.70-3.57 (m, 1H), 2.93-2.89 (m, 1H), 2.63-2.54 (m, 1H), 2.42 (d, J=4.4 Hz, 3H), 1.54-1.49 (m, 3H), 1.23-1.17 (m, 3H). ¹⁹F NMR (376 MHz, DMSO-d₆) δ −103.37.

Compound 42B

LC-MS (ESI): R_{T}=3.558 min, mass calcd. for C₂₆H₂₅Cl₂FN₆O₃ 558.1, m/z found 559.2 [M+H]⁺. Chiral analysis (Column: Chiralpak IE 5 μm 4.6*250 mm; Mobile Phase: Hex:EtOH=30:70 at 1 mL/min; Col. Temp: 30° C.; Wavelength: 254 nm; R_{T}=9.002 min). ¹H NMR (400 MHz, DMSO-d₆) δ 8.57 (s, 1H), 8.06 (s, 1H), 7.75-7.72 (m, 2H), 7.46-7.26 (m, 3H), 5.75-5.65 (m, 1H), 5.41-5.23 (m, 1H), 5.06 (s, 1H), 4.63-4.35 (m, 1H), 4.18 (br s, 1H), 3.82 (br s, 2H), 2.92-2.89 (m, 1H), 2.64 (d, J=4.4 Hz, 3H), 2.59-2.52 (m, 1H), 1.51-1.38 (m, 3H), 1.22-1.11 (m, 3H). ¹⁹F NMR (376 MHz, DMSO-d₆) δ −103.07.

Compounds 43A and 43B

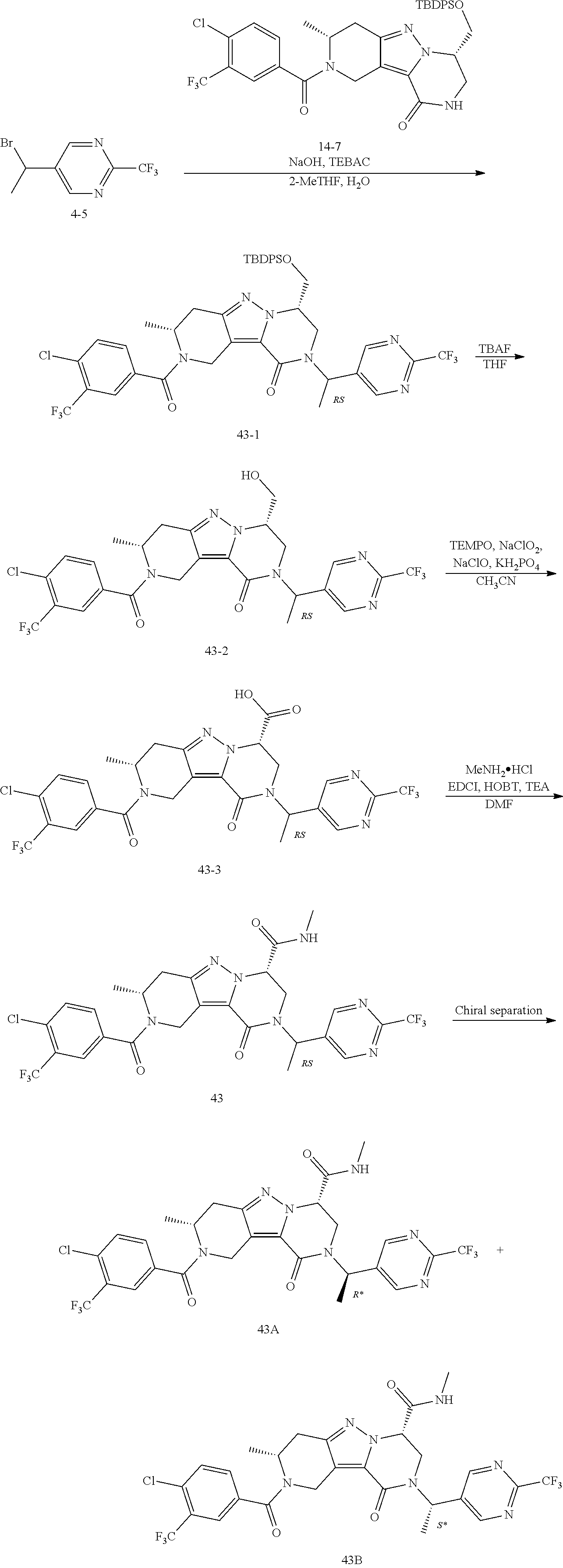

Intermediate 43-1

(3R,7S)-7-(((tert-Butyldiphenylsilyl)oxy)methyl)-2-(4-chloro-3-(trifluoromethyl)benzoyl)-3-methyl-9-(1-(2-(trifluoromethyl)pyrimidin-5-yl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(4-chloro-3-(tri fluoromethyl)benzoyl)-3-methyl-1,2,3,4,8,9-hexahydropyrido[4′,3′:3,4]pyrazolo[1,5-a]pyrazin-10(7H)-one 14-7 (1.5 g, 90% purity, 1.98 mmol) and 5-(1-bromoethyl)-2-(trifluoromethyl)pyrimidine 4-5 (750 mg, 95% purity, 2.79 mmol) in 2-methyltetrahydrofuran (15 mL) was added benzyltriethylammonium chloride (75 mg, 0.33 mmol) and 50% wt. sodium hydroxide in water (7 mL) slowly at 0° C. After being stirred at 30° C. for 1 hour, the reaction mixture was diluted with water (50 mL) and extracted with ethyl acetate (50 mL) twice. The combined organic layers were washed with brine (50 mL) twice, dried over Na₂SO₄₍ₛ₎ and filtered. The filtrate was concentrated under reduced pressure to give a crude. The crude was purified by C18 column (acetonitrile:water=90% to 95%) to give the title compound (270 mg, 100% purity from LCMS, 15.9% yield) as yellow solids. LC-MS (ESI): R_{T}=1.94 min, mass calcd. for C₄₂H₄₁ClF₆N₆O₃Si 854.3, m/z found 855.6 [M+H]⁺.

Intermediate 43-2

(3R,7S)-2-(4-Chloro-3-(trifluoromethyl)benzoyl)-7-(hydroxymethyl)-3-methyl-9-(1-(2-(trifluoromethyl)pyrimidin-5-yl)ethyl)-1,2,3,4,8,9-hexahydropyrido[4′,3′:3,4]pyrazolo[1,5-a]pyrazin-10(7H)-one

To a solution of (3R,7S)-7-(((tert-butyldiphenylsilyl)oxy)methyl)-2-(4-chloro-3-(trifluoromethyl)benzoyl)-3-methyl-9-(1-(2-(trifluoromethyl)pyrimidin-5-yl)ethyl)-1,2,3,4,8,9-hexahydropyrido[4′,3′:3,4]pyrazolo[1,5-a]pyrazin-10(7H)-one 43-1 (320 mg, 100% purity, 0.37 mmol) in tetrahydrofuran (3 mL) was added 1 M tetrabutylammonium fluoride in tetrahydrofuran (0.6 mL, 0.6 mmol) at 0° C. After being stirred at 0° C. for 2 hours, the mixture was concentrated under reduced pressure to give a crude. The crude was purified by C18 column (acetonitrile:water=40% to 55%) to give the title compound (190 mg, 90% purity from ¹H NMR, 74.1% yield) as yellow solids. LC-MS (ESI): R_{T}=1.62 min, mass calcd. for C₂₆H₂₃ClF₆N₆O₃ 616.1, m/z found 617.4 [M+H]⁺. ¹H NMR (400 MHz, CDCl₃) δ 8.93-8.90 (m, 2H), 7.79 (s, 1H), 7.60-7.53 (m, 2H), 6.21-6.03 (m, 1H), 5.69-5.30 (m, 1H), 4.78-4.25 (m, 3H), 4.07-3.66 (m, 3H), 3.46-3.24 (m, 1H), 3.16-2.49 (m, 3H), 1.73 (d, J=6.4 Hz, 3H), 1.28-1.24 (m, 3H).

Intermediate 43-3

(3R,7S)-2-(4-Chloro-3-(trifluoromethyl)benzoyl)-3-methyl-10-oxo-9-(1-(2-(trifluoromethyl)pyrimidin-5-yl)ethyl)-1,2,3,4,7,8,9,10-octahydropyrido[4′,3′:3,4]pyrazolo[1,5-a]pyrazine-7-carboxylic acid

To a solution of (3R,7S)-2-(4-chloro-3-(trifluoromethyl)benzoyl)-7-(hydroxymethyl)-3-methyl-9-(1-(2-(trifluoromethyl)pyrimidin-5-yl)ethyl)-1,2,3,4,8,9-hexahydropyrido[4′,3′:3,4]pyrazolo[1,5-a]pyrazin-10(7H)-one 43-2 (190 mg, 90% purity, 0.28 mmol) in acetonitrile (4 mL) were added 2,2,6,6-tetramethylpiperidinooxy (90 mg, 0.58 mmol), sodium chlorite (65 mg, 80% purity, 0.58 mmol), saturated potassium dihydrogenphosphate aqueous solution (4 mL) and sodium hypochlorite aqueous solution (0.33 mL, 10% purity, 0.55 mmol) at 0° C. After being stirred at 0° C. for 2 hours, the mixture was diluted with sodium sulfite saturated solution (20 mL) and extracted with ethyl acetate (30 mL) twice. The combined organic layers were washed with brine (30 mL), dried over Na₂SO₄₍ₛ₎ and filtered. The filtrate was concentrated to give the title compound (190 mg, 90.2% purity from LCMS, 99.0% yield) as yellow solids. LC-MS (ESI): R_{T}=1.33 min, mass calcd. for C₂₆H₂₁ClF₆N₆O₄ 630.1, m/z found 631.3 [M+H]⁺.

Compound 43

(3R,7S)-2-(4-Chloro-3-(trifluoromethyl)benzoyl)-N,3-dimethyl-10-oxo-9-(1-(2-(trifluoromethyl)pyrimidin-5-yl)ethyl)-1,2,3,4,7,8,9,10-octahydropyrido[4′,3′:3,4]pyrazolo[1,5-a]pyrazine-7-carboxamide

To the mixture of (3R,7S)-2-(4-chloro-3-(trifluoromethyl)benzoyl)-3-methyl-10-oxo-9-(1-(2-(trifluoromethyl)pyrimidin-5-yl)ethyl)-1,2,3,4,7,8,9,10-octahydropyrido[4′,3′:3,4]pyrazolo[1,5-a]pyrazine-7-carboxylic acid 43-3 (190 mg, 90% purity, 0.27 mmol), methanamine hydrochloride (40 mg, 0.59 mmol), 1-ethyl-(3-(3-dimethylamino)propyl)-carbodiimide hydrochloride (110 mg, 0.57 mmol) and benzotriazol-1-ol (80 mg, 0.59 mmol) in N,N-dimethylformamide (5 mL) at 0° C. was added trimethylamine (0.2 mL, 1.42 mmol). After being stirred at 0° C. under nitrogen atmosphere for 2 hours, the mixture was acidified to pH=6 with 0.5 M hydrochloride aqueous solution and extracted with ethyl acetate (50 mL) twice. The combined organic layers were washed with water (30 mL) three times and brine (30 mL), dried over Na₂SO₄₍ₛ₎ and filtered. The filtrate was concentrated under reduced pressure to give a residue, which was purified by C18 column (acetonitrile:water=55% to 75%) to give the title compound (97 mg, 100% purity from LCMS, 55.6% yield) as white solids. LC-MS (ESI): R_{T}=1.60 min, mass calcd. for C₂₇H₂₄ClF₆N₇O₃ 643.2, m/z found 644.4 [M+H]⁺.

Compounds 43A and 43B

(3R,7S)-2-(4-Chloro-3-(trifluoromethyl)benzoyl)-N,3-dimethyl-10-oxo-9-((R*)-1-(2-(trifluoromethyl)pyrimidin-5-yl)ethyl)-1,2,3,4,7,8,9,10-octahydropyrido[4′,3′:3,4]pyrazolo[1,5-a]pyrazine-7-carboxamide (43A), and (3R,7S)-2-(4-chloro-3-(trifluoromethyl)benzoyl)-N,3-dimethyl-10-oxo-9-((S*)-1-(2-(trifluoromethyl)pyrimidin-5-yl)ethyl)-1,2,3,4,7,8,9,10-octahydropyrido[4′,3′:3,4]pyrazolo[1,5-a]pyrazine-7-carboxamide (43B)

A racemic mixture of (3R,7S)-2-(4-chloro-3-(trifluoromethyl)benzoyl)-N,3-dimethyl-10-oxo-9-(1-(2-(trifluoromethyl)pyrimidin-5-yl)ethyl)-1,2,3,4,7,8,9,10-octahydropyrido[4′,3′:3,4]pyrazolo[1,5-a]pyrazine-7-carboxamide 43 (97 mg, 97.8% purity, 0.21 mmol) was separated by chiral Prep. HPLC (separation conditon: Column: Chiralpak IE 5 μm 20*250 mm; Mobile Phase: Hex:EtOH=30:70 at 25 mL/min; Temp: 30° C.; Wavelength: 254 nm) to give the title compound 43A (20 mg, 99.6% purity from LCMS, 20.5% yield, 100% stereopure) and compound 43B (25 mg, 99.8% purity from LCMS, 25.7% yield, 100% stereopure) as white solids.

Compound 43A

LC-MS (ESI): R_{T}=4.214 min, mass calcd. for C₂₇H₂₄ClF₆N₇O₃ 643.2, m/z found 644.2 [M+H]⁺. Chiral analysis (Column: Chiralpak IC 5 μm 4.6*250 mm; Mobile Phase: Hex:EtOH=30:70 at 1 mL/min; Col. Temp: 30° C.; Wavelength: 254 nm; Rt=5.239 min). ¹H NMR (400 MHz, CDCl₃) δ 8.84 (s, 2H), 7.77 (s, 1H), 7.59-7.55 (m, 2H), 5.99-5.50 (m, 3H), 4.94-4.88 (m, 1H), 4.78-4.30 (m, 2H), 4.12-4.09 (m, 1H), 3.95-3.90 (m, 1H), 3.15-2.99 (m, 1H), 2.76-2.68 (m, 4H), 1.71 (d, J=7.2 Hz, 3H), 1.33 (d, J=6.4 Hz, 3H). ¹⁹F NMR (376 MHz, CDCl₃) δ −62.85, −70.25.

Compound 43B

LC-MS (ESI): R_{T}=4.288 min, mass calcd. for C₂₇H₂₄ClF₆N₇O₃ 643.2, m/z found 644.2 [M+H]⁺. Chiral analysis (Column: Chiralpak IC 5 μm 4.6*250 mm; Mobile Phase: Hex:EtOH=30:70 at 1 mL/min; Col. Temp: 30° C.; Wavelength: 254 nm; Rt=8.959 min). ¹H NMR (400 MHz, CDCl₃) δ 8.92 (s, 2H), 7.77 (s, 1H), 7.61-7.52 (m, 2H), 6.11-5.36 (m, 3H), 4.93-4.30 (m, 4H), 3.65-3.61 (m, 1H), 3.18-2.96 (m, 1H), 2.82 (d, J=4.8 Hz, 3H), 2.75-2.65 (m, 1H), 1.76 (d, J=7.2 Hz, 3H), 1.30 (d, J=6.8 Hz, 3H). ¹⁹F NMR (376 MHz, CDCl₃) δ −62.84, −70.29.

Compounds 44A and 44B

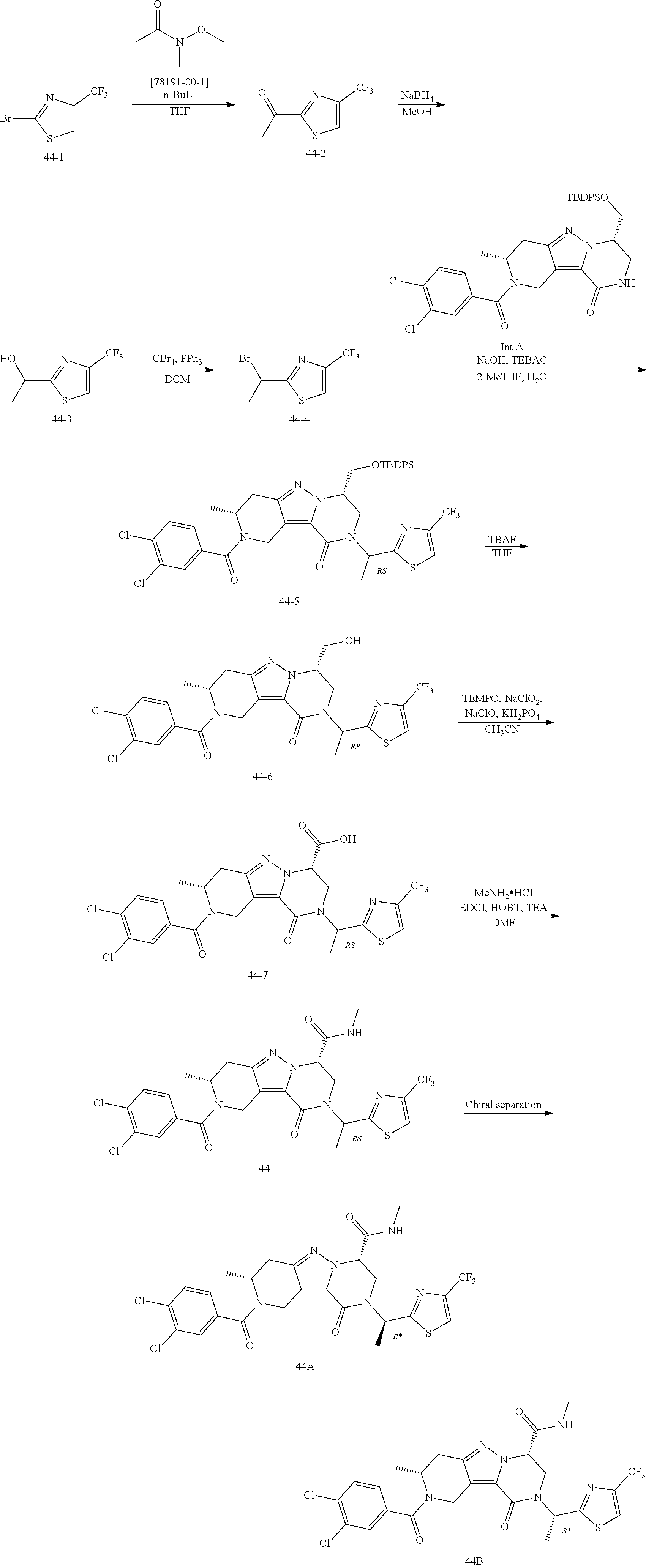

Intermediate 44-2

1-(4-(Trifluoromethyl)thiazol-2-yl)ethanone

To a solution of 2-bromo-4-(trifluoromethyl)thiazole 44-1 (2.90 g, 12.5 mmol) in tetrahydrofuran (40 mL) was cooled to −60° C., then 2.5 M n-butyllithium in tetrahydrofuran (6 mL, 15.0 mmol) was added dropwise at −60° C. and the mixture was stirred at −60° C. for 1 hour. Then, N-methoxy-N-methylacetamide (1.90 g, 18.4 mmol) was added dropwise at −60° C. After being stirred at −60° C. for 2 hours and room temperature for 16 hours, the reaction mixture was cooled to 0° C. and quenched by saturated ammonium chloride aqueous solution (100 mL) and extracted with ethyl acetate (100 mL) for three times. The combined organic layers were washed by brine (100 mL), dried over Na₂SO₄₍ₛ₎, filtered and concentrated under reduced pressure to give a residue, which was purified by silica gel column (petroleum ether:ethyl acetate=5:1) to give the title compound (2.00 g, 87% purity from ¹H NMR, 71% yield) as a yellow solid. ¹H NMR (400 MHz, CDCl₃) δ 8.07-8.05 (m, 1H), 2.75 (s, 3H).

Intermediate 44-3

1-(4-(Trifluoromethyl)thiazol-2-yl)ethanol

The solution of 1-(4-(trifluoromethyl)thiazol-2-yl)ethanone 44-2 (2.00 g, 8.92 mmol) in methanol (20 mL) was cooled to 0° C., then sodium borohydride (670 mg, 17.7 mmol) was added. The reaction mixture was stirred at 0° C. for 2 hours and quenched by water (2 mL) and concentrated to give a residue, which was purified by silica gel column (dichloromethane:methanol=15:1) to give the title compound (1.70 g, 71% purity from LCMS, 69% yield) as a yellow solid. LC-MS (ESI): R_{T}=1.05 min, mass calcd. for C₆H₆F₃NOS 197.0, m/z found 198.0 [M+H]⁺. ¹H NMR (400 MHz, CDCl₃) δ 8.39-8.37 (m, 1H), 6.39-6.34 (m, 1H), 5.02-4.94 (m, 1H), 1.47 (d, J=6.8 Hz, 3H).

Intermediate 44-4

2-(1-Bromoethyl)-4-(trifluoromethyl)thiazole

The solution of 1-(4-(trifluoromethyl)thiazol-2-yl)ethanol 44-3 (1.20 g, 71% purity, 3.07 mmol) in dichloromethane (20 mL) was cooled to 0° C. Then triphenylphosphine (1.70 g, 6.48 mmol) and tetrabromomethane (2.10 g, 6.33 mmol) were added. After being stirred at 0° C. to room temperature for 8 hours, the reaction mixture was concentrated to give a residue, which was purified by silica gel column (petroleum ether:ethyl acetate=5:1) to give the title compound (700 mg, 90% purity from ¹H NMR, 56% yield) as a yellow solid. ¹H NMR (400 MHz, CDCl₃) δ 7.79-7.78 (m, 1H), 5.44-5.38 (m, 1H), 2.16 (d, J=6.8 Hz, 311).

Intermediate 44-5

(3R,7S)-7-(((tert-Butyldiphenylsilyl)oxy)methyl)-2-(3,4-dichlorobenzoyl)-3-methyl-9-(1-(4-(trifluoromethyl)thiazol-2-yl)ethyl)-1,2,3,4,8,9-hexahydropyrido[4′,3′:3,4]pyrazolo[1,5-a]pyrazin-10(7H)-one

To a mixture of 2-(1-bromoethyl)-4-(trifluoromethyl)thiazole 44-4 (400 mg, 1.38 mmol) and (3R,7S)-7-(((tert-butyldiphenylsilyl)oxy)methyl)-2-(3,4-dichlorobenzoyl)-3-methyl-1,2,3,4,8,9-hexahydropyrido[4′,3′:3,4]pyrazolo[1,5-a]pyrazin-10(7H)-one Int A (950 mg, 1.35 mmol) in 2-methyltetrahydrofuran (15 mL) and 50% wt. sodium hydroxide aqueous solution (10 mL) was added benzyltriethylammonium chloride (100 mg, 0.42 mmol) at 0° C. The reaction mixture was stirred at 0° C. to room temperature for 3 hours. The reaction mixture was diluted with ethyl acetate (100 mL) and the organic layer was washed by saturated ammonium chloride aqueous solution (50 mL) and brine (50 mL). The organic layer was dried over Na₂SO₄₍ₛ₎, filtered and concentrated under reduced pressure to give a residue, which was purified by silica gel column (dichloromethane:methanol=12:1) to give the title compound (1.20 g, 67% purity from LCMS, 70% yield) as a yellow solid. LC-MS (ESI): R_{T}=1.895 min, mass calcd. for C₄₀H₄₀Cl₂F₃N₅O₃SSi 825.2, m/z found 825.9 [M+H]⁺.

Intermediate 44-6

(3R,7S)-2-(3,4-Dichlorobenzoyl)-7-(hydroxymethyl)-3-methyl-9-(1-(4-(trifluoromethyl)thiazol-2-yl)ethyl)-1,2,3,4,8,9-hexahydropyrido[4′,3′:3,4]pyrazolo[1,5-a]pyrazin-10(7H)-one

The solution of (3R,7S)-7-(((tert-butyldiphenylsilyl)oxy)methyl)-2-(3,4-dichlorobenzoyl)-3-methyl-9-(1-(4-(trifluoromethyl)thiazol-2-yl)ethyl)-1,2,3,4,8,9-hexahydropyrido[4′,3′:3,4]pyrazolo[1,5-a]pyrazin-10(7H)-one 44-5 (1.20 g, 67% purity, 0.974 mmol) in tetrahydrofuran (20 mL) was cooled to 0° C., then 1 M tetrabutylammonium fluoride in tetrahydrofuran (1.5 mL, 1.50 mmol) was added dropwise. After being stirred at 0° C. for 1 hour, the reaction mixture was concentrated under reduced pressure and purified by silica gel column (dichloromethane:methanol=15:1) to afford the title compound (550 mg, 95% purity from LCMS, 91% yield) as a yellow solid. LC-MS (ESI): R_{T}=1.44 min, mass calcd. for C₂₄H₂₂Cl₂F₃N₅O₃S 587.1, m/z found 587.9 [M+H]⁺. ¹H NMR (400 MHz, CDCl₃) δ 7.81-7.76 (m, 1H), 7.53-7.50 (m, 2H), 7.25-7.24 (m, 1H), 6.36-5.38 (m, 2H), 4.87-4.32 (m, 3H), 4.08-4.00 (m, 1H), 3.98-3.72 (m, 3H), 3.66-3.59 (m, 1H), 3.12-2.98 (m, 1H), 2.73-2.65 (m, 1H), 1.76-1.74 (m, 3H), 1.28-1.24 (m, 3H).

Intermediate 44-7

(3R,7S)-2-(3,4-Dichlorobenzoyl)-3-methyl-10-oxo-9-(1-(4-(trifluoromethyl)thiazol-2-yl)ethyl)-1,2,3,4,7,8,9,10-octahydropyrido[4′,3′:3,4]pyrazolo[1,5-a]pyrazine-7-carboxylic acid

To a suspension of (3R,7S)-2-(3,4-dichlorobenzoyl)-7-(hydroxymethyl)-3-methyl-9-(1-(4-(trifluoromethyl)thiazol-2-yl)ethyl)-1,2,3,4,8,9-hexahydropyrido[4′,3′:3,4]pyrazolo[1,5-a]pyrazin-10(7H)-one 44-6 (550 mg, 95% purity, 0.890 mmol) in acetonitrile (5 mL) was added saturated potassium dihydrogen phosphate aqueous solution (5 mL). The mixture was cooled to 0° C., then 2,2,6,6-tetramethylpiperidinyloxy (347 mg, 2.22 mmol), sodium chlorite (251 mg, 2.22 mmol) and sodium hypochlorite aqueous solution (1.3 mL, 2.18 mmol) was added. After being stirred at room temperature for 16 hours, the reaction mixture was adjusted pH to 5-6 by 1 M hydrochloride aqueous solution and extracted with dichloromethane (50 mL) for three times. The combined organic layers were washed by brine (50 mL), dried over Na₂SO₄₍ₛ₎, filtered and concentrated under reduced pressure to give a residue, which was purified by silica gel column (dichloromethane:methanol=5:1) to give the title compound (480 mg, 92% purity from LCMS, 83% yield) as a yellow solid. LC-MS (ESI): R_{T}=1.211 min, mass calcd. for C₂₄H₂₀Cl₂F₃N₅O₄S 601.1, m/z found 601.9 [M+H]⁺.

Compound 44

(3R,7S)-2-(3,4-Dichlorobenzoyl)-N,3-dimethyl-10-oxo-9-(1-(4-(trifluoromethyl)thiazol-2-yl)ethyl)-1,2,3,4,7,8,9,10-octahydropyrido[4′,3′:3,4]pyrazolo[1,5-a]pyrazine-7-carboxamide

To a mixture of (3R,7S)-2-(3,4-dichlorobenzoyl)-3-methyl-10-oxo-9-(1-(4-(tri fluoromethyl)thiazol-2-yl)ethyl)-1,2,3,4,7,8,9,10-octahydropyrido[4′,3′:3,4]pyrazolo[1,5-a]pyrazine-7-carboxylic acid 44-7 (480 mg, 92% purity, 0.730 mmol) and methylamine hydrochloride (100 mg, 1.48 mmol) in dimethylformamide (8 mL) was added 1-hydroxybenzotrizole (198 mg, 1.47 mmol) and 1-(3-dimethylaminopropyl)-3-ethylcarbodiimide hydrochloride (281 mg, 1.47 mmol). After being cooled down to 0° C., a solution of triethylamine (223 mg, 2.20 mmol) in N, N-dimethylformamide (1 mL) was added dropwise within 30 minutes. The reaction mixture was stirred at 0° C. for 1 hour. The reaction mixture was quenched by saturated ammonium chloride aqueous solution (20 mL) and extracted with ethyl acetate (50 mL) for three times. The combined organic layers were washed by water (50 mL) for three times and brine (50 mL), dried over Na₂SO₄₍ₛ₎, filtered and concentrated under reduced pressure, which was purified by silica gel column (dichloromethane:methanol=12:1) to give the title compound (350 mg, 98% purity from LCMS, 76% yield) as a yellow solid. LC-MS (ESI): R_{T}, =1.41 min, mass calcd. for C₂₅H₂₃Cl₂F₃N₆O₃S 614.1, m/z found 614.9 [M+H]⁺.

Compounds 44A and 44B

(3R,7S)-2-(3,4-Dichlorobenzoyl)-N,3-dimethyl-10-oxo-9-((R*)-1-(4-(trifluoromethyl)thiazol-2-yl)ethyl)-1,2,3,4,7,8,9,10-octahydropyrido[4′,3′:3,4]pyrazolo[1,5-a]pyrazine-7-carboxamide (44A), and (3R,7S)-2-(3,4-dichlorobenzoyl)-N,3-dimethyl-10-oxo-9-((S*)-1-(4-(trifluoromethyl)thiazol-2-yl)ethyl)-1,2,3,4,7,8,9,10-octahydropyrido[4′,3′:3,4]pyrazolo[1,5-a]pyrazine-7-carboxamide (44B)

A racemic mixture of (3R,7S)-2-(3,4-dichlorobenzoyl)-N,3-dimethyl-10-oxo-9-(1-(4-(trifluoromethyl)thiazol-2-yl)ethyl)-1,2,3,4,7,8,9,10-octahydropyrido[4′,3′:3,4]pyrazolo[1,5-a]pyrazine-7-carboxamide 44 (350 mg, 98% purity, 0.560 mmol) was purified by chiral Prep. HPLC (Column: Chiralpak IA 5 μm 20*250 mm; Mobile Phase: Hex:EtOH=30:70 at 30 mL/min; Temp: 30° C.; Wavelength: 254 nm) to give the title compound 44A (87.4 mg, 96.2% purity from LCMS, 24.5% yield, 100% stereopure) as a white solid and compound 44B (116 mg, 98.9% purity from LCMS, 33.5% yield, 99.9% stereopure) as a white solid.

Compound 44A

LC-MS (ESI): R_{T}=3.844 min, mass calcd. for C₂₅H₂₃Cl₂F₃N₆O₃S 614.1, m/z found 615.0 [M+H]⁺. Chiral analysis (Column: Chiralpak IA 5 μm 4.6*250 mm; Mobile Phase: Hex:EtOH=30:70 at 1.0 mL/min; Temp: 30° C.; Wavelength: 254 nm; R_{T}=6.112 min). ¹H NMR (400 MHz, CDCl₃) δ 7.78 (s, 1H), 7.54-7.51 (m, 2H), 7.28-7.26 (m, 1H), 6.13-5.41 (m, 3H), 4.93-4.32 (m, 3H), 4.21-4.15 (m, 1H), 4.00-3.97 (m, 1H), 3.12-2.98 (m, 1H), 2.78-2.71 (m, 4H), 1.77-1.75 (m, 3H), 1.32-1.30 (m, 3H).

Compound 44B

LC-MS (ESI): R_{T}=3.916 min, mass calcd. for C₂₅H₂₃Cl₂F₃N₆O₃S 614.1, m/z found 615.1 [M+H]⁺. Chiral analysis (Column: Chiralpak IA 5 μm 4.6*250 mm; Mobile Phase: Hex:EtOH=30:70 at 1.0 mL/min; Temp: 30° C.; Wavelength: 254 nm; R_{T}=8.405 min). ¹H NMR (400 MHz, CDCl₃) δ 7.74 (s, 1H), 7.52-7.50 (m, 2H), 7.25-7.23 (m, 1H), 6.23-5.35 (m, 3H), 4.95-4.38 (m, 4H), 3.88-3.82 (m, 1H), 3.15-2.94 (m, 1H), 2.82-2.70 (m, 4H), 1.77-1.75 (m, 3H), 1.30-1.28 (m, 3H).

Compound 45

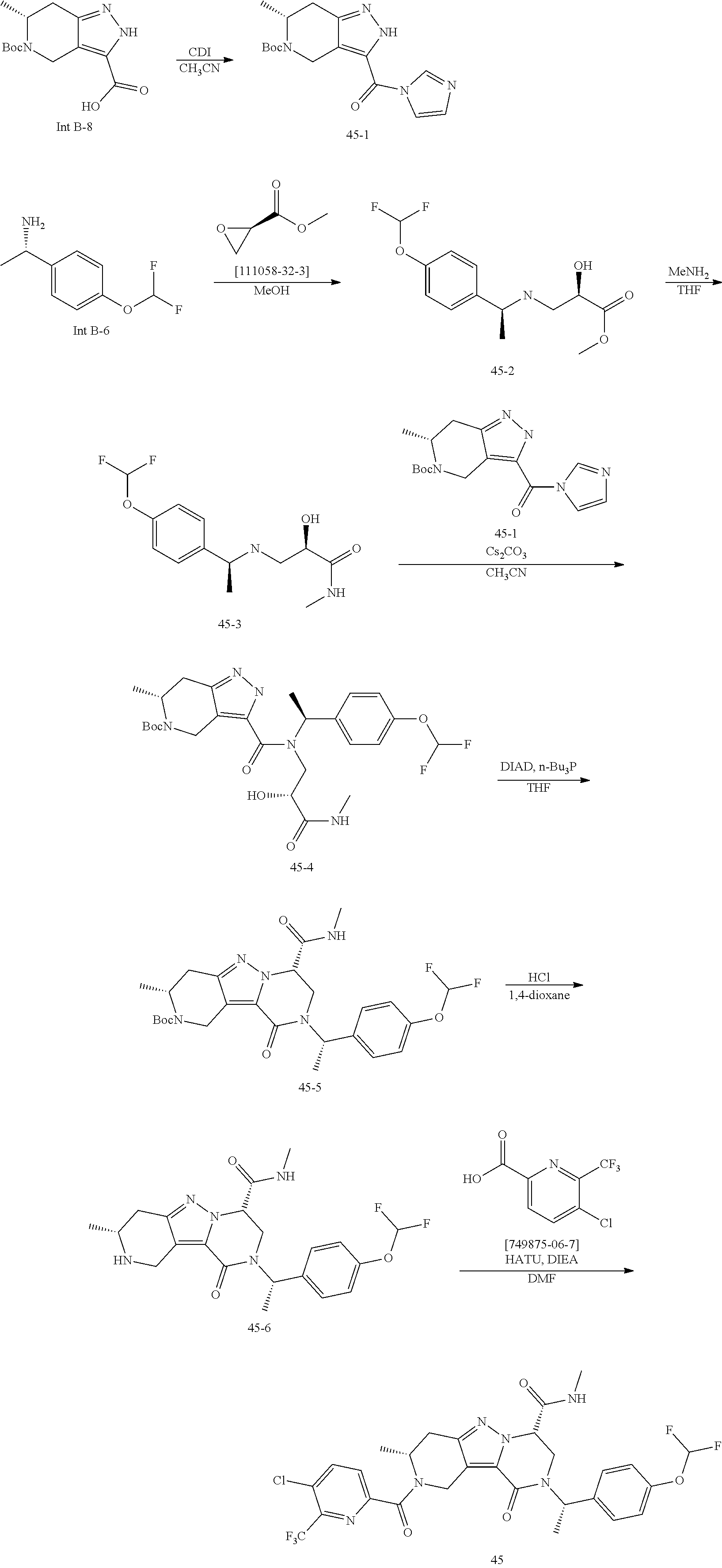

Intermediate 45-1

tert-Butyl (R)-3-(1H-imidazole-1-carbonyl)-6-methyl-2,4,6,7-tetrahydro-5H-pyrazolo[4,3-c]pyridine-5-carboxylate

To a solution of (R)-5-(tert-butoxycarbonyl)-6-methyl-4,5,6,7-tetrahydro-2H-pyrazolo[4,3-c]pyridine-3-carboxylic acid Int B-8 (20 g, 100% purity, 71.1 mmol) in acetonitrile (250 mL) was added di(1H-imidazol-1-yl)methanone (12 g, 74.0 mmol) at 0° C. After being stirred at 0° C. for 1 hour, the mixture was added water (100 mL), and ethyl acetate (200 mL). The organic layers were washed by brine (200 mL), dried over Na₂SO₄₍ₛ₎ and filtered. The filtrate was concentrated to give the title compound (21 g, 100% purity, 89.1% yield) as a light white solid. LC-MS (ESI): R_{T}=1.50 min, mass calcd. for C₁₆H₂₁N₅O₃ 331.4, m/z found 332.1 [M+H]⁺.

Intermediate 45-2

Methyl (R)-3-(((S)-1-(4-(difluoromethoxy)phenyl)ethyl)amino)-2-hydroxypropanoate

To the solution of (S)-1-(4-(difluoromethoxy)phenyl)ethan-1-amine Int B-6 (4.2 g, 95% purity, 21.3 mmol) in methanol (40 mL) was added methyl (R)-oxirane-2-carboxylate (2.28 g, 23.3 mmol) at 30° C. in a seal tube. After being stirred at 60° C. for 24 hours, LCMS showed 43% starting material remained. Then, the mixture was added methyl (R)-oxirane-2-carboxylate (1.0 g, 9.8 mmol) at 30° C. After being stirred at 60° C. for 19 hours, LCMS showed 34% starting material remained. The mixture was concentrated under reduced pressure and added water (10 mL). The aqueous layer was extracted with ethyl acetate (30 mL) three times. The combined organic layers were dried over Na₂SO₄₍ₛ₎ and filtered. The filtrate was concentrated under reduced pressure to give the title compound (1.5 g, 50% purity from LCMS, 12% yield) as yellow oil. LC-MS (ESI): R_{T}=1.385 min, mass calcd. for C₁₃H₁₇F₂NO₄ 289.1, m/z found 290.2 [M+H]⁺.

Intermediate 45-3

(R)-3-(((S)-1-(4-(Difluoromethoxy)phenyl)ethyl)amino)-2-hydroxy-N-methylpropanamide

To methyl (R)-3-(((S)-1-(4-(difluoromethoxy)phenyl)ethyl)amino)-2-hydroxypropanoate 45-2 (1.5 g, 50% purity, 2.59 mmol) was added 2 M methylamine in tetrahydrofuran (3 mL, 6 mmol) at 30° C. After being stirred at 70° C. for 48 hours, the mixture was concentrated under reduced pressure and purified by C18 column (acetonitrile:water=5% to 100%) to give the crude, which was purified by column gel column chromatography (dichloromethane:methanol=19:1) to give the title compound (540 mg, 80% purity from LCMS, 58% yield) as yellow oil. LC-MS (ESI): R_{T}=1.25 min, mass calcd. for C₁₃H₁₈F₂N₂O₃ 288.1, m/z found 289.1 [M+H]⁺.

Intermediate 45-4

tert-Butyl (R)-3-(((S)-1-(4-(difluoromethoxy)phenyl)ethyl)((R)-2-hydroxy-3-(methylamino)-3-oxopropyl)carbamoyl)-6-methyl-2,4,6,7-tetrahydro-5H-pyrazolo[4,3-c]pyridine-5-carboxylate

To a solution of (R)-3-(((S)-1-(4-(difluoromethoxy)phenyl)ethyl)amino)-2-hydroxy-N-methylpropanamide 45-3 (440 mg, 80% purity, 1.22 mmol) in acetonitrile (9 mL) was added tert-butyl (R)-3-(1H-imidazole-1-carbonyl)-6-methyl-2,4,6,7-tetrahydro-5H-pyrazolo[4,3-c]pyridine-5-carboxylate 45-1 (485 mg, 1.46 mmol) and cesium carbonate (796 mg, 2.44 mmol) at 0° C. under nitrogen atmosphere. After being stirred at 45° C. for 4 hours, the mixture was concentrated, purified by silica gel column chromatography (dichloromethane methanol=19:1) to give the title compound (430 mg, 93% purity from LCMS, 59% yield) as white solids. LC-MS (ESI): R_{T}=1.15 min, mass calcd. for C₂₆H₃₅F₂N₅O₆ 551.3, m/z found 552.2 [M+H]⁺.

Intermediate 45-5

tert-Butyl (3R,7S)-9-((S)-1-(4-(difluoromethoxy)phenyl)ethyl)-3-methyl-7-(methylcarbamoyl)-10-oxo-3,4,7,8,9,10-hexahydropyrido[4′,3′:3,4]pyrazolo[1,5-a]pyrazine-2(1H)-carboxylate

To a solution of tert-butyl (R)-3-(((S)-1-(4-(difluoromethoxy)phenyl)ethyl)((R)-2-hydroxy-3-(methylamino)-3-oxopropyl)carbamoyl)-6-methyl-2,4,6,7-tetrahydro-5H-pyrazolo[4,3-c]pyridine-5-carboxylate 45-4 (430 mg, 93% purity, 0.73 mmol) in tetrahydrofuran (16 mL) was added tributylphosphine (0.26 mL, 1.04 mmol) and diisopropyl (E)-diazene-1,2-dicarboxylate (0.22 mL, 1.12 mmol) at 0° C. under nitrogen atmosphere. After being stirred at room temperature for 2 hours, the mixture was concentrated, purified by silica gel column chromatography (dichloromethane:methanol=20:1) to give the title compound (340 mg, 98% purity from LCMS, 86% yield) as white solids. LC-MS (ESI): R_{T}=1.203 min, mass calcd. for C₂₆H₃₃F₂N₅O₅ 533.2, m/z found 534.2 [M+H]⁺.

Intermediate 45-6

(3R,7S)-9-((S)-1-(4-(Difluoromethoxy)phenyl)ethyl)-N,3-dimethyl-10-oxo-1,2,3,4, 7,8,9,10-octahydropyrido[4′,3′:3,4]pyrazolo[1,5-a]pyrazine-7-carboxamide

To a solution of tert-butyl (3R,7S)-9-((S)-1-(4-(difluoromethoxy)phenyl)ethyl)-3-methyl-7-(methylcarbamoyl)-10-oxo-3,4,7,8,9,10-hexahydropyrido[4′,3′:3,4]pyrazolo [1,5-a]pyrazine-2(1H)-carboxylate 45-5 (290 mg, 98% purity, 0.53 mmol) in dichloromethane (5 mL) was added 4 M hydrochloride in 1,4-dioxane (2.5 mL, 10 mmol) at 0° C. After being stirred at room temperature for 1 hour, the reaction mixture was washed with saturated sodium bicarbonate and extracted with dichloromethane (10 mL) twice. The combined organic layers were dried over Na₂SO₄₍ₛ₎ and filtered. The filtrate was concentrated under reduced pressure to give the title compound (200 mg, 95% purity from ¹H NMR, 82% yield) as yellow solids, which was purified by C18 column (acetonitrile: pure water=5% to 66%) to give the title compound (42.5 mg, 98% purity from LCMS, 22% yield, 100% stereopure) as white solids. LC-MS (ESI): R_{T}=2.427 min, mass calcd. for C₂₁H₂₅F₂N₅O₃ 433.2, m/z found 434.1 [M+H]⁺. Chiral analysis (Column: Chiralpak IB N-5 5 μm 4.6*250 mm; Mobile Phase: ACN=100% at 1 mL/min; Col. Temp: 30° C.; Wavelength: 254 nm at R_{T}=5.632 min). ¹H NMR (400 MHz, DMSO-d₆) δ 7.93-7.89 (m, 1H), 7.41-7.04 (m, 5H), 5.77 (q, J=J=6.8 Hz, 1H), 4.96-4.94 (m, 1H), 4.10-4.06 (m, 1H), 3.79-3.75 (m, 1H), 3.66-3.62 (m, 1H), 3.51-3.46 (m, 1H), 2.81-2.73 (m, 1H), 2.62-2.58 (m, 4H), 2.22-2.16 (m, 2H), 1.40 (d, J=7.2 Hz, 3H), 1.15 (d, J=6.0 Hz, 3H). ¹⁹F NMR (376 MHz, DMSO-d₆) δ −82.04.

Compound 45

(3R,7S)-2-(5-Chloro-6-(trifluoromethyl)picolinoyl)-9-((S)-1-(4-(difluoromethoxy) phenyl)ethyl)-N,3-dimethyl-10-oxo-1,2,3,4,7,8,9,10-octahydropyrido[4′,3′:3,4]pyrazolo[1,5-a]pyrazine-7-carboxamide

To a mixture of (3R,7S)-9-((S)-1-(4-(difluoromethoxy)phenyl)ethyl)-N,3-dimethyl-10-oxo-1,2,3,4,7,8,9,10-octahydropyrido[4′,3′:3,4]pyrazolo[1,5-a]pyrazine-7-carboxamide 45-6 (9 mg, 99.3% purity, 0.02 mmol), 5-chloro-6-(trifluoromethyl)picolinic acid (6.5 mg, 0.03 mmol) and HATU (12 mg, 0.03 mmol) in N,N-dimethylformamide (0.5 mL) was added N-ethyl-N-isopropylpropan-2-amine (0.01 mL, 0.06 mmol) at 0° C. After being stirred at room temperature for 1 hour, the reaction mixture was added water (1 mL) and extracted with ethyl acetate (2 mL) for three times. The organic layers were washed with brine (1 mL) and the filtrate was concentrated and purified by C18 column (acetonitrile:water=5% to 95%) to give the title compound (4 mg, 97.7% purity from LCMS, 29.6% yield) as white solids. LC-MS (ESI): R_{T}=3.722 min, mass calcd. for C₂₈H₂₆ClF₅N₆O₄ 640.2, m/z found 641.1 [M+H]⁺. ¹H NMR (400 MHz, CDCl₃) δ 8.02-7.89 (m, 2H), 7.40-7.33 (m, 2H), 7.13-7.09 (m, 2H), 6.50 (t, J=73.6 Hz, 1H), 6.09-5.28 (m, 3H), 4.86-4.63 (m, 2H), 4.45-4.40 (m, 1H), 4.11-4.01 (m, 1H), 3.40-3.11 (m, 2H), 2.80 (d, J=4.8 Hz, 3H), 2.75-2.67 (m, 1H), 1.60-1.57 (m, 3H), 1.37-1.31 (m, 3H). ¹⁹F NMR (376 MHz, CDCl₃) δ −66.04, −66.17, −80.99, −81.04.

Compound 46

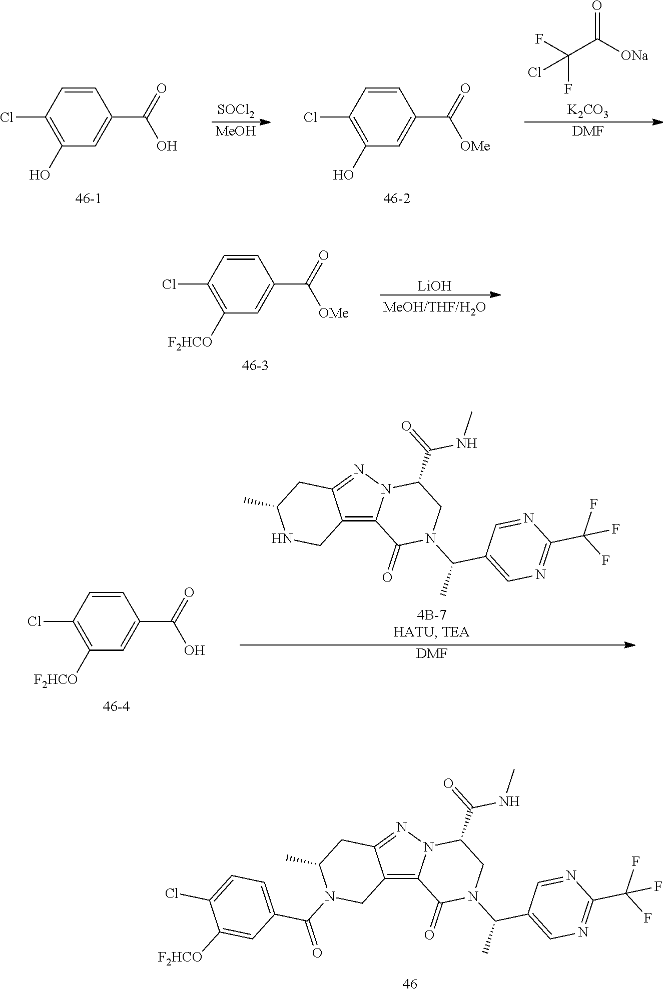

Intermediate 46-2

Methyl 4-chloro-3-hydroxybenzoate

To a solution of 4-chloro-3-hydroxybenzoic acid 46-1 (3.0 g, 17.4 mmol) in methanol (30 mL) was added sulfurous dichloride (2.48 g, 20.9 mmol) dropwise at 0° C. The resulting mixture was stirred at 0° C. for 10 minutes, then heated to 60° C. and stirred for 2 hours. After cooled to the room temperature, the reaction mixture was concentrated to afford a residue, which was dissolved in ethyl acetate (50 mL), washed with 5% wt. sodium bicarbonate aqueous solution (20 mL), brine (20 mL), dried over Na₂SO₄₍ₛ₎ and filtered. The filtrate was concentrated in vacuo to give the title product (3.20 g, 96.4% purity from LCMS, 95% yield) as yellow solids. LC-MS (ESI): R_{T}=1.391 min, mass calcd. for C₈H₇ClO₃ 186.0 m/z found 373.1 [2M+H]⁺.

Intermediate 46-3

Methyl 4-chloro-3-(difluoromethoxy)benzoate

To a solution of methyl 4-chloro-3-hydroxybenzoate 46-2 (3.20 g, 17.2 mmol) and potassium carbonate (2.84 g, 20.6 mmol) in N,N-dimethylformamide (50 mL) was added sodium 2-chloro-2,2-difluoroacetate (3.14 g, 20.6 mmol). The resulting mixture was stirred at 80° C. overnight. After cooled down to room temperature, the reaction mixture was poured into water (200 mL), extracted with ethyl acetate (100 mL) for three times. The combined organic layers were washed with brine (100 mL), dried over Na₂SO₄₍ₛ₎ and filtered. The filtrate was concentrated in vacuo to afford a residue, which was purified by column chromatography on silica gel (petroleum ether:ethyl acetate=8:1) to give the title product (2.0 g, 90% purity from ¹NMR, 44% yield) as yellow solids. ¹H NMR (400 MHz, CDCl₃) δ 7.89 (s, 1H), 7.85 (dd, J=8.4 and 1.6 Hz, 1H), 7.53 (d, J=8.4 Hz, 1H), 6.59 (t, J=72.8 Hz, 1H), 3.93 (s, 3H).

Intermediate 46-4

4-Chloro-3-(difluoromethoxy)benzoic acid

To a solution of methyl 4-chloro-3-(difluoromethoxy)benzoate 46-3 (2.0 g, 90% purity, 7.61 mmol) in methanol (5 mL) and tetrahydrofuran (15 mL) was added a solution of lithium hydroxide monohydrate (638.5 mg, 15.2 mmol) in water (10 mL) at 20° C. The resulting mixture was stirred at 20° C. for 2 hours, then poured into water (50 mL) and extracted with ethyl acetate (20 mL) twice. The aqueous layer was acidified by 10% wt. hydrochloride aqueous solution to pH=4 and extracted with dichloromethane (20 mL) for three times. The combined dichloromethane organic layers were washed with brine (10 mL), dried over Na₂SO₄₍ₛ₎ and filtered. The filtrate was concentrated in vacuo to give the title product (1.85 g, 90% purity from ¹NMR, 98.3% yield) as yellow solids. ¹H NMR (400 MHz, CDCl₃) δ 7.97 (s, 1H), 7.93 (dd, J=8.4 and 1.6 Hz, 1H), 7.58 (d, J=8.4 Hz, 1H), 6.61 (t, J=72.8 Hz, 1H).

Compound 46

(3R,7S)-2-(4-chloro-3-(difluoromethoxy)benzoyl)-N,3-dimethyl-10-oxo-9-((S)-1-(2-(trifluoromethyl)pyrimidin-5-yl)ethyl)-1,2,3,4,7,8,9,10-octahydropyrido [4′,3′:3,4]pyrazolo[1,5-a]pyrazine-7-carboxamide

To the mixture of (3R,7S)—N,3-dimethyl-10-oxo-9-((S)-1-(2-(trifluoromethyl) pyrimidin-5-yl)ethyl)-1,2,3,4,7,8,9,10-octahydropyrido[4′,3′:3,4]pyrazolo[1,5-a]pyrazine-7-carboxamide (contain 20% isomer (3R,7S)—N,3-dimethyl-10-oxo-9-((R)-1-(2-(trifluoromethyl) pyrimidin-5-yl)ethyl)-1,2,3,4,7,8,9,10-octahydropyrido[4′,3′:3,4]pyrazolo[1,5-a]pyrazine-7-carboxamide) 4B-7 (150 mg, 78.9% purity, 0.271 mmol) and 4-chloro-3-(difluoromethoxy)benzoic acid 46-4 (80 mg, 90% purity, 0.33 mmol) in N,N-dimethylformamide (2 mL) was added 2-(3H-[1,2,3]triazolo[4,5-b]pyridin-3-yl)-1,1,3,3-tetramethylisouronium hexafluoro phosphate (V) (134 mg, 0.35 mmol) at room temperature. After stirred at 0° C. for 10 minutes, triethylamine (82 mg, 0.81 mmol) was added dropwise at 0° C. Then the stirring was continued at 0° C. for 3 hours. The reaction mixture was quenched with brine (10 mL), extracted with acetate (10 mL) for three times, dried over Na₂SO₄₍ₛ₎, filtered. The filtrate was concentrated to get a residue, which was purified by C18 chromatography (acetonitrile:water (+0.02% ammonium bicarbonate)=40-60%) to give the title compound mixture (85.2 mg, 96.5% purity from LCMS, 47.4% yield) as white solids. LC-MS (ESI): R_{T}=3.073 min, mass calcd. for C₂₇H₂₅ClF₅N₇O₄ 641.2 m/z found 642.1 [M+H]⁺. The mixture was further purified with chiral HPLC (Column: Chiral ART Cellulose-SC 10 m 50*250 mm; Mobile Phase: ACN=100 at 100 mL/min; Col. Temp: 25° C.; Wavelength: 254 nm) to afford the title compound 46 (40 mg, 95.1% purity from LCMS, 44.9% yield, 99.4% stereopure) as white solids. 46: LC-MS (ESI): R_{T}=3.080 min, mass calcd. for C₂₇H₂₅ClF₅N₇O₄ 641.2 m/z found 642.1 [M+H]⁺. Chiral analysis (Column: Chiral ART Cellulose-SC 10 μm 4.6*250 mm; Mobile Phase: ACN=100 at 1 mL/min; Col. Temp: 25° C.; Wavelength: 254 nm, R_{T}=5.067 min). ¹H NMR (400 MHz, CDCl₃) δ 8.92 (s, 2H), 7.54-7.52 (m, 1H), 7.31 (s, 1H), 7.26-7.24 (m, 1H), 6.57 (t, J=72.8 Hz, 1H), 6.07 (br s, 1H), 5.85 (br s, 1H), 5.69-5.30 (m, 1H), 4.94 (s, 1H), 4.82-4.30 (m, 3H), 3.65-3.60 (m, 1H), 3.04 (br s, 1H), 2.82 (d, J=4.8 Hz, 3H), 2.74-2.70 (m, 1H), 1.75 (d, J=7.2 Hz, 3H), 1.30-1.29 (m, 3H). ¹⁹F NMR (376 MHz, CDCl₃) δ −70.28, −81.58-−82.77.

Compound 47

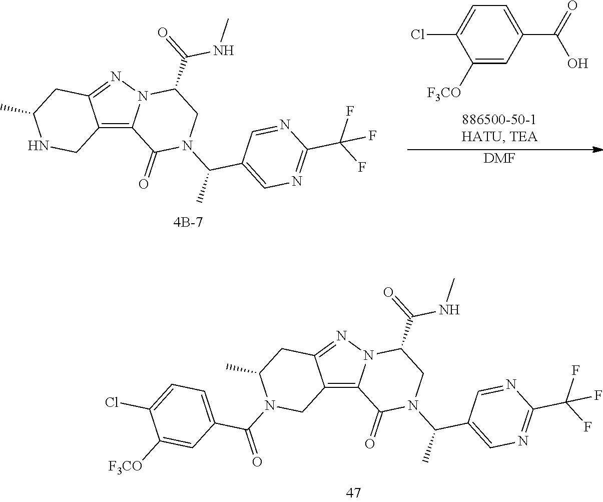

Compound 47

(3R,7S)-2-(4-chloro-3-(trifluoromethoxy)benzoyl)-N,3-dimethyl-10-oxo-9-((S)-1-(2-(trifluoromethyl)pyrimidin-5-yl)ethyl)-1,2,3,4,7,8,9,10-octahydropyrido [4′,3′:3,4]pyrazolo[1,5-a]pyrazine-7-carboxamide

To a mixture of (3R,7S)—N,3-dimethyl-10-oxo-9-((S)-1-(2-(trifluoromethyl) pyrimidin-5-yl)ethyl)-1,2,3,4,7,8,9,10-octahydropyrido[4′,3′:3,4]pyrazolo[1,5-a]pyrazine-7-carboxamide (contain 20% isomer (3R,7S)—N,3-dimethyl-10-oxo-9-((R)-1-(2-(trifluoromethyl) pyrimidin-5-yl)ethyl)-1,2,3,4,7,8,9,10-octahydropyrido[4′,3′:3,4]pyrazolo[1,5-a]pyrazine-7-carboxamide) 4B-7 (150 mg, 78.9% purity, 0.27 mmol) and 4-chloro-3-(trifluoromethoxy)benzoic acid (78 mg, 0.33 mmol) in N,N-dimethylformamide (2 mL) was added 2-(3H-[1,2,3]triazolo[4,5-b]pyridin-3-yl)-1,1,3,3-tetramethylisouronium hexafluorophosphate(V) (134 mg, 0.35 mmol). After stirred at 0° C. for 10 minutes, triethylamine (82 mg, 0.81 mmol) was added dropwise to the reaction at 0° C. Then stirring was continued at 0° C. for 3 hours. The reaction mixture was quenched with brine (10 mL), extracted with acetate (10 mL) for three times, dried over Na₂SO₄₍ₛ₎, filtered. The filtrate was concentrated to get a residue, which was purified by C18 chromatography (acetonitrile:water (+0.02% ammonium bicarbonate)=40-60%) to give a mixture (123 mg, 97.1% purity from LCMS, 67.6% yield) as white solids. LC-MS (ESI): R_{T}=3.307 min, mass calcd. for C₂₇H₂₄ClF₆N₇O₄ 659.2 m/z found 660.1 [M+H]⁺. The mixture was further purified with chiral HPLC (Column: Chiral ART Cellulose-SC 10 μm 50*250 mm; Mobile Phase: ACN=100 at 100 mL/min; Col. Temp: 25° C.; Wavelength: 254 nm) to afford the title compound 47 (35 mg, 28% yield, 95% purity from ¹HNMR, 99.7% stereopure) as white solids.

47

LC-MS (ESI): R_{T}=10.84 min, mass calcd. for C₂₇H₂₄ClF₆N₇O₄ 659.2 m/z found 660.6 [M+H]⁺. Chiral analysis (Column: Chiral ART Cellulose-SC 10 m 4.6*250 mm; Mobile Phase: ACN=100 at 1 mL/min; Col. Temp: 25° C.; Wavelength: 254 nm, R_{T}=5.345 min). ¹H NMR (400 MHz, CDCl₃) δ 8.92 (s, 2H), 7.57 (d, J=8.4 Hz, 1H), 7.39 (s, 1H), 7.32 (dd, J=8.4 and 2.0 Hz, 1H), 6.09 (br s, 1H), 5.84-5.45 (m, 2H), 4.94 (br s, 1H), 4.77-4.47 (m, 2H), 4.32 (d, J=12.4 Hz, 1H), 3.63 (d, J=12.4 Hz, 1H), 3.08 (br s, 1H), 2.82 (d, J=4.8 Hz, 3H), 2.75-2.71 (m, 1H), 1.75 (d, J=7.2 Hz, 3H), 1.29 (d, J=6.4 Hz, 3H). ¹⁹F NMR (376 MHz, CDCl₃) δ −57.77, −70.28.

Biological Examples

### Anti-HBV Activity of Compounds of Formula (D

### Procedure

The anti HBV activity was measured using the HepG2.117 cell line, a stable, inducibly HBV producing cell line, which replicates HBV in the absence of doxycycline (Tet-off system). The HepG2 cell line is available from ATCCR under number HB-8065. Transfection of the HepG2 cell line can be as described in Sun and Nassal 2006 Journal of Hepatology 45 (2006) 636-645 “Stable HepG2-and Huh7-based human hepatoma cell lines for efficient regulated expression of infectious hepatitis B virus”.

For the antiviral assay, HBV replication was induced, followed by a treatment with serially diluted compound in 96-well plates. After 3 days of treatment, the antiviral activity was determined by quantification of intracellular HBV DNA using real-time PCR and an HBV specific primer set and probe.

| | | |
|---|---|---|
| Forward Primer | GTGTCTGCGGCGTTTTATCA | SEQ ID. No: 1 |
| Reverse Primer | GACAAACGGGCAACATACCTT | SEQ ID. No: 2 |
| Taqman Probe | CCTCTKCATCCTGCTGCTATGCCTCATC_FAM- | SEQ ID. No: 3 |
| | BHQ1 | |

Cytotoxicity of the compounds was tested using HepG2 or HepG2.117 cells, incubated for 3 or 4 days in the presence of compounds. The viability of the cells was assessed using the PERKIN ELMER ATPlite Luminescence Assay System.”

Human Liver Microsome Stability t½ (Min) of Compounds of Formula (I)

### Procedure

Pooled human liver microsomes (0.5 mg/mL) was pre-incubated with 1 μM test compounds or control compounds (verapamil, warfarin and cerivastatin) in 0.1 M phosphate buffer pH 7.4 containing 1 mM MgCl₂ at 37° C. The final concentration of DMSO and acetonitrile are 0.05% and 0.2%, respectively. All incubations are performed singularly for each compound. NADPH was added to initiate the reaction with the final concentration of 1 mM and the final incubation volume of 400 μL.

At 6 time points (0, 5, 10, 20, 40 and 60 min) reactions were stopped by the removal of 50 μL of the incubation mixture into plates containing acetonitrile at a of 1:3 (v:v). The plates were centrifuged at 3000 rpm for 10 min at 4° C. to precipitate the protein. Following protein precipitation, the sample supernatants were combined in cassettes of up to 8 compounds. The internal standard was added (1:1 supernatant to internal standard solution) and samples were analyzed using standard LC-MS conditions.

From a plot of ln peak area ratio (compound peak area/internal standard peak area) against time, the gradient of the line is determined. Subsequently, half-life is calculated using the equations below:

$$Elimination rate constant ( k ) = ( - gradient )$$

$$Half - life ( t 1 / 2 ) ( min ) = 0.693 / k$$

### Results

N.A.=not available
CC50 values: 3-days incubation unless marked with*(*=4-days incubation)

TABLE 4

| Compound | EC50 (μM, | CC50 (μM, | HLM Metabolism t_{1/2} |
|---|---|---|---|
| Number | mean value) | mean value) | (min) |
| 1B | 0.38 | >50.0 | >180 |
| 2A | 0.042 | >50.0 | >180 |
| 2B | 0.017 | >50.0 | >180 |
| 3B | 0.051 | >50.0 | >180 |
| 4A | 2.443 | >50.0 | >180 |
| 4B | 0.16 | >50.0 | >180 |
| 5B | 0.019 | >50.0 | 86.2 |
| 6B | 0.092 | >50.0 | >180 |
| 7B | 0.049 | >50.0 | >180 |
| 8B | 0.071 | >50.0 | >180 |
| 9B | 0.061 | >50.0 | >180 |
| 10B | 0.019 | 46.82 | >180 |
| 11A | 0.12 | >50.0 | >180 |
| 11B | 0.052 | 23.42 | >180 |
| 12A | 0.059 | 24.43 | >180 |
| 12B | 0.027 | 27.38 | >180 |
| 13A | 0.34 | >50.0 | >180 |
| 13B | 0.033 | 46.82 | >180 |
| 14B | 0.031 | >50.0 | >180 |
| 15A | 0.086 | >50.0 | >180 |
| 15B | 0.029 | 29.81 | >180 |
| 16A | 0.021 | 40.40 | >180 |
| 16B | 0.020 | 16.92 | >180 |
| 17 | 0.036 | 39.06 | >180 |
| 18A | 0.597 | >50.0 | >180 |
| 18B | 0.037 | >50.0 | >180 |
| 19A | 0.120 | >50.0 | >180 |
| 19B | 0.028 | >50.0 | >180 |
| 20A | 0.089 | >50.0 | >180 |
| 20B | 0.022 | >50.0 | >180 |
| 21 | 0.086 | >50.0 | >180 |
| 22A | 0.270 | >50.0 | >180 |
| 22B | 0.050 | >50.0 | >180 |
| 23A | 0.060 | >50.0 | >180 |
| 23B | 0.023 | >50.0 | >180 |
| 24A | 0.054 | 20.58 | >180 |
| 24B | 0.013 | 25.53 | >180 |
| 25 | 0.010 | >50.0 | 77.9 |
| 26A | 0.190 | 29.74 | >180 |
| 26B | 0.090 | >50.0 | 67.2 |
| 27A | 0.031 | >50.0 | >180 |
| 27B | 0.046 | >50.0 | >180 |
| 28 | 0.019 | 29.53 | >180 |
| 29A | 0.820 | >50.0 | >180 |
| 29B | 0.082 | >50.0 | >180 |
| 30A | 0.016 | >50.0 | >180 |
| 30B | 0.017 | 17.24 | 90.9 |
| 31A | 0.066 | >50.0 | >180 |
| 31B | 0.018 | >50.0 | >180 |
| 32A | 0.055 | >50.0 | 127 |
| 32B | 0.011 | 48.69 | >180 |
| 33A | 0.062 | >50.0 | 47.9 |
| 33B | 0.055 | >50.0 | >180 |
| 34A | 0.380 | >50.0 | 110 |
| 34B | 0.049 | >50.0 | >180 |
| 35A | 0.390 | >50.0 | 69.5 |
| 35B | 0.082 | >50.0 | 130 |
| 36A | 0.086 | >50.0 | >180 |
| 36B | 0.050 | >50.0 | >180 |
| 37A | 0.089 | >50.0 | >180 |
| 37B | 0.067 | >50.0 | >180 |
| 38A | 0.068 | >50.0 | >180 |
| 38B | 0.040 | >50.0 | 144 |
| 39A | 1.676 | >50.0 | >180 |
| 39B | 0.120 | >50.0 | >180 |
| 40A | 0.280 | >50.0 | >180 |
| 40B | 0.097 | >50.0 | >180 |
| 41A | 2.212 | >50.0 | >180 |
| 41B | 0.100 | >50.0 | >180 |
| 42A | 0.280 | >50.0 | >180 |
| 42B | 0.020 | >50.0 | >180 |
| 43A | 0.610 | >50.0 | >180 |
| 43B | 0.057 | >50.0 | >180 |
| 44A | 0.120 | >50.0 | >180 |
| 44B | 0.091 | >50.0 | >180 |
| 45 | 0.067 | 49.8 | >180 |
| 46 | N.A. | N.A. | N.A. |
| 47 | N.A. | N.A. | N.A. |
| Ref 1 | 0.069 | >50 | 15.4 |
| Ref 2 | 0.026 | 19.84 | 7.05 |
| Ref 3 | 0.072 | >50 | 6.6 |
| Ref 4 | 0.076 | >50 | 13.5 |
| Ref 5 | 0.022 | >50 | 7.9 |

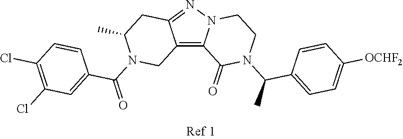 [SMILES: C[C@@H]1Cc2nn3c(c2CN1C(=O)c1ccc(Cl)c(Cl)c1)C(=O)N([C@H](C)c1ccc(OC(F)F)cc1)CC3]

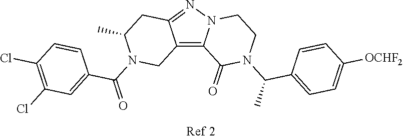 [SMILES: C[C@@H]1Cc2nn3c(c2CN1C(=O)c1ccc(Cl)c(Cl)c1)C(=O)N([C@@H](C)c1ccc(OC(F)F)cc1)CC3]

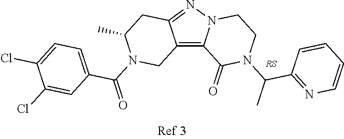 [SMILES: CC(c1ccccn1)N1CCn2nc3c(c2C1=O)CN(C(=O)c1ccc(Cl)c(Cl)c1)[C@H](C)C3]

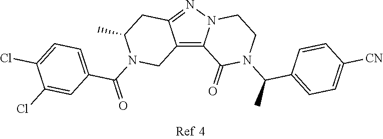 [SMILES: C[C@@H]1Cc2nn3c(c2CN1C(=O)c1ccc(Cl)c(Cl)c1)C(=O)N([C@H](C)c1ccc(C#N)cc1)CC3]

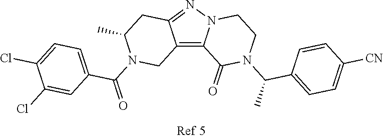 [SMILES: C[C@@H]1Cc2nn3c(c2CN1C(=O)c1ccc(Cl)c(Cl)c1)C(=O)N([C@@H](C)c1ccc(C#N)cc1)CC3]

Ref 1, Ref 2, Ref 3, Ref 4 and Ref 5 can be prepared according to the procedures of WO2020/243135.

According to the experimental results, the present compounds showed improved human liver microsome stability as well as reasonable anti-HBV activity. As compared to comparative compounds (e.g., reference compounds 1-5), the half-life (t½) of the present compounds are significantly increased, showing great improvement in human metabolic stability.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims. All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A compound of Formula (I), 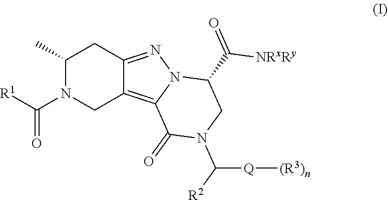
or a stereoisomeric or a tautomeric form thereof, wherein
R¹ is selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₁₋₆alkoxyl, C₃₋₆cycloalkyl and CN, each of C₁₋₆alkyl, C₁₋₆alkoxyl and C₃₋₆cycloalkyl is optionally substituted with 1, 2, 3, 4 or 5 substituents independently selected from the group consisting of halo, hydroxyl and CN;
R² is selected from the group consisting of H, CHF₂, CF₃, C₁₋₆alkyl, C₁₋₆alkylOC₁₋₆alkyl and C₃₋₆cycloalkyl;
Q represents a ring selected from the group consisting of phenyl, 5-membered heteroaryl and 6-membered heteroaryl;
n represents 0, 1, 2 or 3;
each R³ independently represents a substituent selected from the group consisting of CN, C₁₋₆alkyl, C₁₋₆alkoxyl, C₃₋₆cycloalkyl, 5-membered heteroaryl, 6-membered heteroaryl, 4-8 membered heterocyclyl, halo, N(R_{S})₂, S(O)R_{S} and S(O)₂R_{S}, each of C₁₋₆alkyl, C₁₋₆alkoxyl, C₃₋₆cycloalkyl, 5-membered heteroaryl, 6-membered heteroaryl, 4-8 membered heterocyclyl, N(R_{S})₂, S(O)R_{S} and S(O)₂R_{S} is optionally substituted with 1, 2, 3, 4 or 5 substituents, each of said substituents independently selected from the group consisting of halo, hydroxyl, C₁₋₆alkyl and oxo; R_{S} is each independently selected from the group consisting of H, C₁₋₆alkyl, C₁₋₆alkoxyl and C₃₋₆cycloalkyl;
Rₓ and R_{y} are each independently selected from the group consisting of hydrogen, C₁₋₆alkyl, and C₃₋₆cycloalkyl,
or a pharmaceutically acceptable salt or a solvate thereof;
wherein the following compounds are excluded: 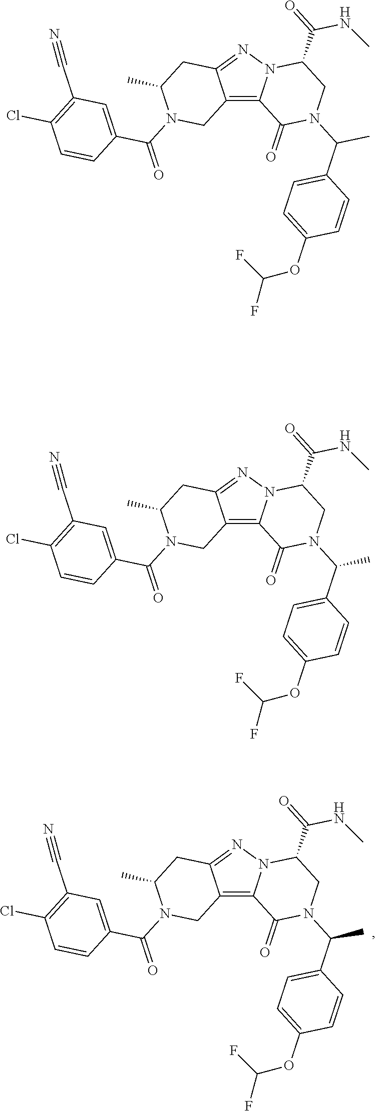 [SMILES: CNC(=O)[C@@H]1CN(C(C)c2ccc(OC(F)F)cc2)C(=O)c2c3c(nn21)C[C@@H](C)N(C(=O)c1ccc(Cl)c(C#N)c1)C3 ; CNC(=O)[C@@H]1CN([C@H](C)c2ccc(OC(F)F)cc2)C(=O)c2c3c(nn21)C[C@@H](C)N(C(=O)c1ccc(Cl)c(C#N)c1)C3 ; CNC(=O)[C@@H]1CN([C@@H](C)c2ccc(OC(F)F)cc2)C(=O)c2c3c(nn21)C[C@@H](C)N(C(=O)c1ccc(Cl)c(C#N)c1)C3]
or a pharmaceutically acceptable salt or a solvate thereof.

2. The compound of claim 1, wherein R¹ is a ring selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₃₋₆cycloalkyl, CF₃, CHF₂, OCHF₂, CN and OCF₃.

3. The compound of claim 2, wherein R¹ is phenyl or pyridyl, which is substituted with 1, 2 or 3 substituents, each of said substituents independently selected from the group consisting of halo, C₁₋₆alkyl, CF₃, CN, and CHF₂.

4. The compound of claim 1, wherein the structural unit 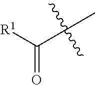
in Formula (I) satisfies Formula (Ia) 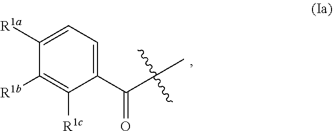
wherein R^{1a}, R^{1b}, and R^{1c}, each independently are selected from the group consisting of hydrogen, halo, C₁₋₆alkyl, C₃₋₆cycloalkyl, CF₃, CHF₂, OCHF₂, CN and OCF₃, with at least one of R^{1a}, R^{1b}, and R^{1c} not being hydrogen.

5. The compound of claim 4, wherein R^{1a} and R^{1b} are independently selected from the group consisting of halo, CN, CHF₂, CF₃, OCHF₂ and OCF₃, and wherein R^{1c} is hydrogen.

6. The compound of claim 1, wherein R² is selected from the group consisting of CHF₂, CF₃, C₁₋₆alkyl, C₁₋₆alkylOC₁₋₆alkyl and C₃₋₆cycloalkyl;
the structure of Formula (I) has Formula (I-1) or Formula (I-2)

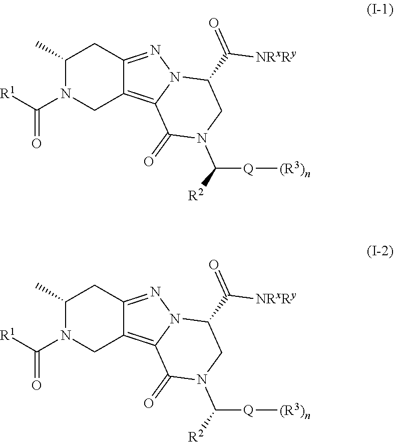

7. The compound of claim 1, wherein R² is methyl or ethyl.

8. The compound of claim 1, wherein Q is a ring selected from the group consisting of phenyl, pyridyl, pyrimidinyl, pyridazinyl, pyrazinyl, pyrazolyl, imidazolyl, and thiazolyl.

9. The compound of claim 1, wherein the structural unit 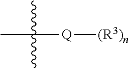
in Formula (I) satisfies Formula (Ib) 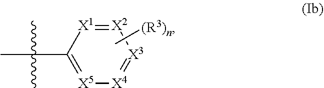
wherein
all of X¹, X², X³, X⁴ and X⁵ are CH; or
one or two of X¹, X², X³, X⁴ and X⁵ are N, and rest of them are CH.

10. The compound of claim 9, wherein the structural unit 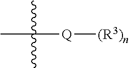
of Formula (I) satisfies Formula (Ic) 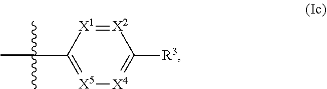
wherein
all of X¹, X², X⁴ and X⁵ are CH;
X² is N, and X¹, X⁴ and X⁵ are CH; or
X¹ is N, and X², X⁴ and X⁵ are CH.

11. The compound of claim 9, wherein
both of X¹ and X² are N, and X⁴ and X⁵ are CH;
both of X² and X⁴ are N, and X¹ and X⁵ are CH;
both of X¹ and X⁴ are N, and X² and X⁵ are CH; or
both of X¹ and X⁵ are N, and X² and X⁴ are CH.

12. The compound of claim 1, wherein the structural unit 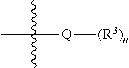
in Formula (I) of satisfies Formula (Ib′) 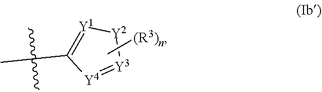
wherein one or two of Y¹, Y², Y³ and Y⁴ are S, N or NH, and rest of them are C.

13. The compound of claim 1, wherein halo is F, Cl or Br.

14. The compound of claim 1, wherein n is 1 or 2.

15. The compound of claim 1, wherein one of R^{x} and R^{Y} is hydrogen, and the other is C₁₋₆alkyl or C₃₋₆cycloalkyl; or
R^{x} and R are both H or C₁₋₆alkyl; or
R^{x} and R are each independently selected from the group consisting of C₁₋₆alkyl, and C₃₋₆cycloalkyl.

16. The compound of claim 1, wherein one R³ is independently selected from the group consisting of halo, CH₃, 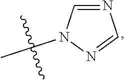 [SMILES: *n1cncn1]
C(CH₃)₂OH, CH₂CF₃, CF₃, OCHF₂, SO₂CH₃, 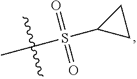 [SMILES: *S(=O)(=O)C1CC1]
OCH₃, CN, 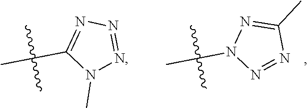
cyclopropyl, NHCH₂CF₃, 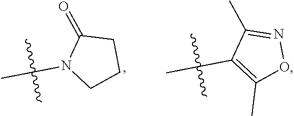
CHF₂, and

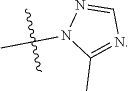 [SMILES: *n1ncnc1C]

17. A compound of claim 1, selected from the group consisting of the following compounds, or a stereoisomeric or a tautomeric form thereof: 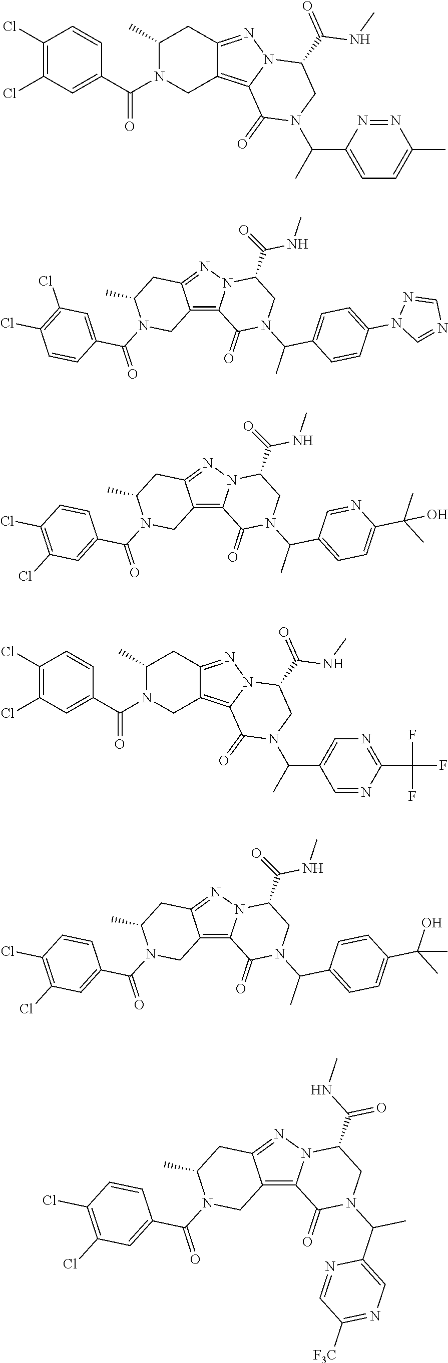 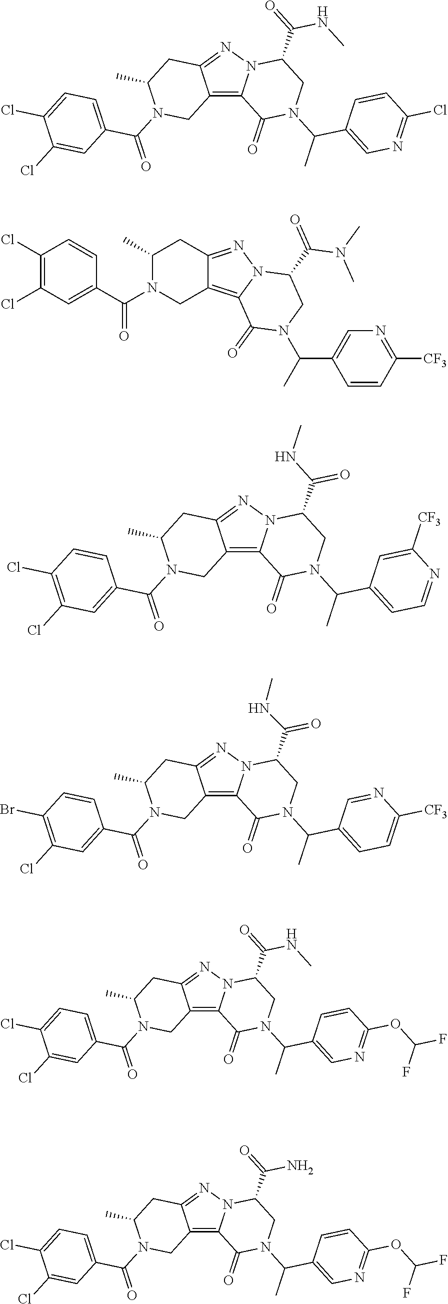 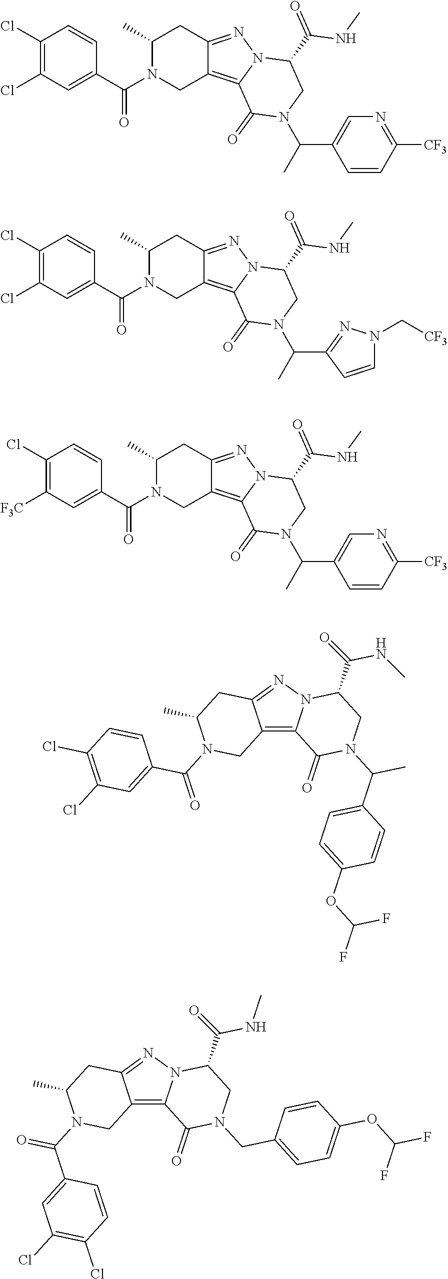 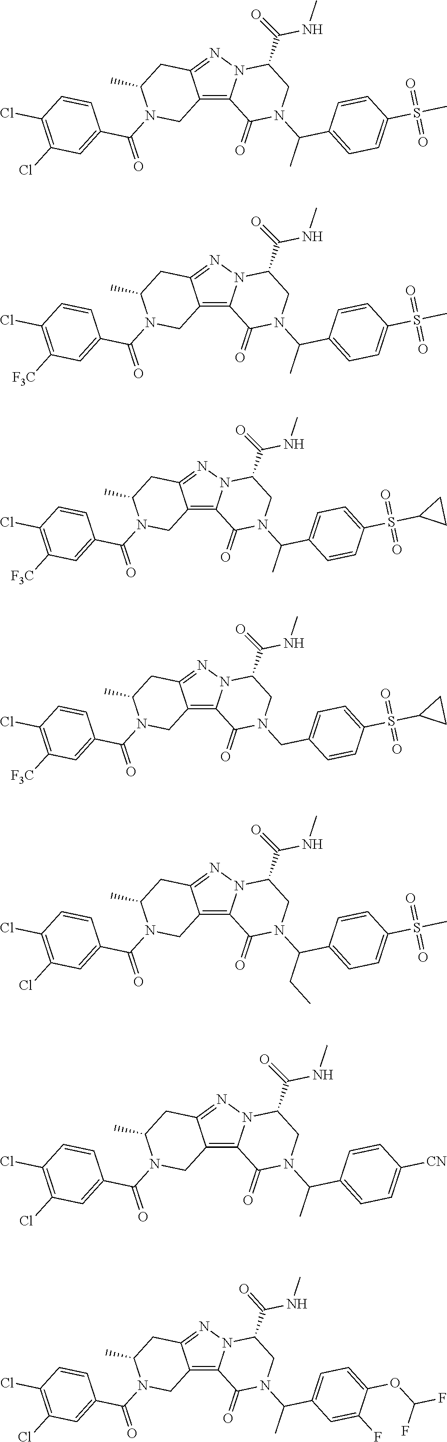 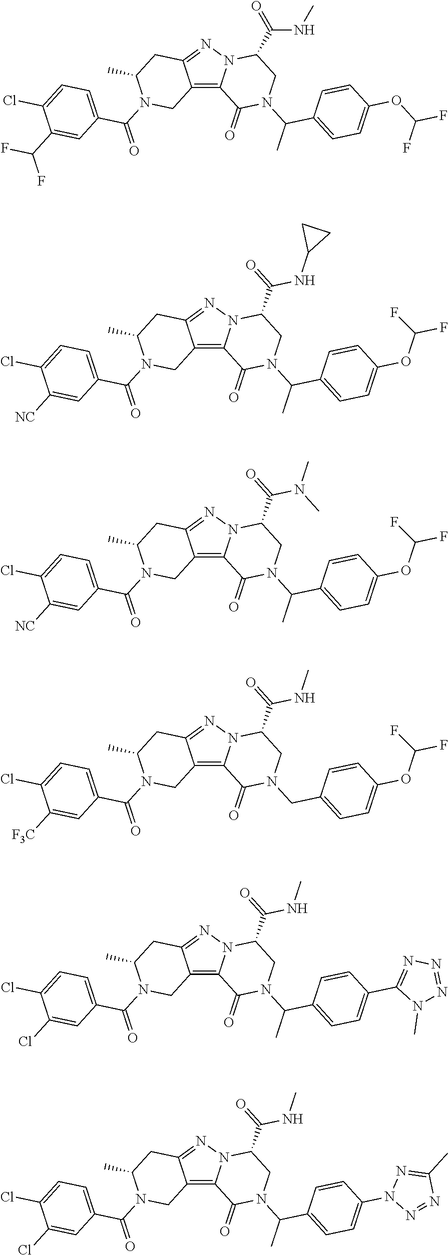 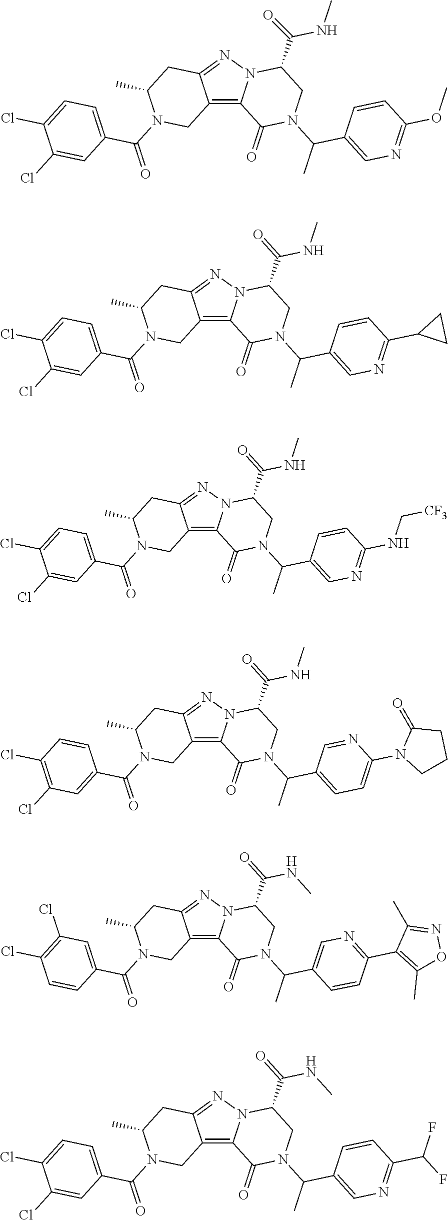 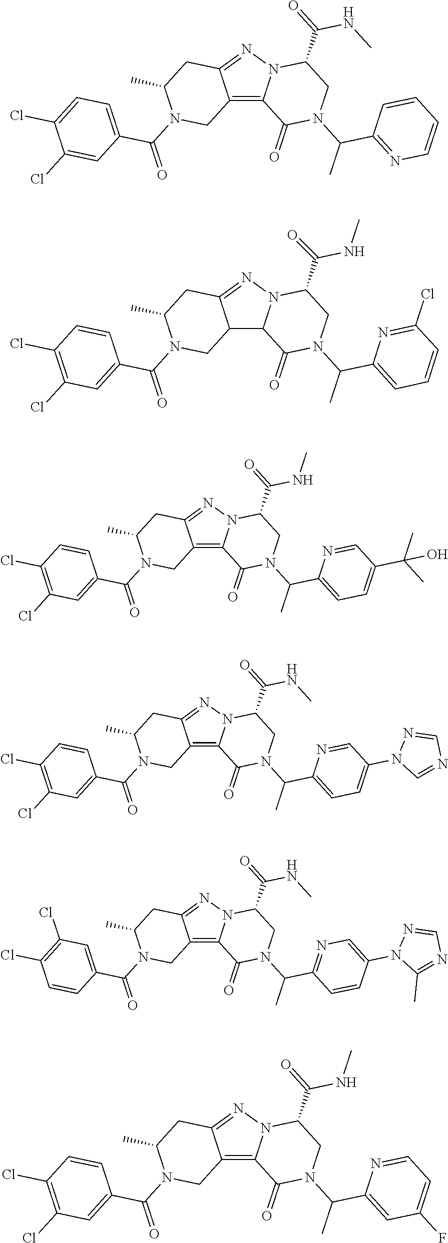 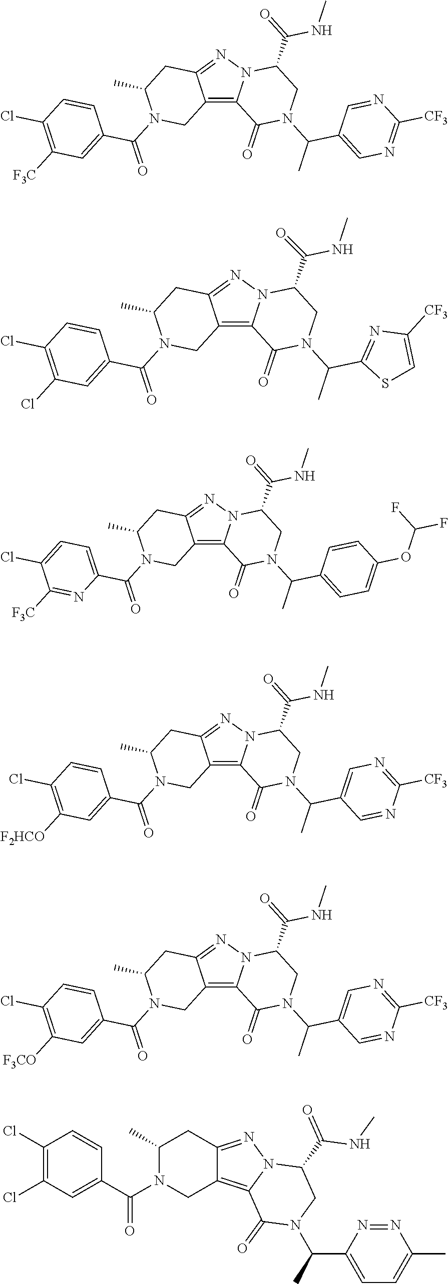 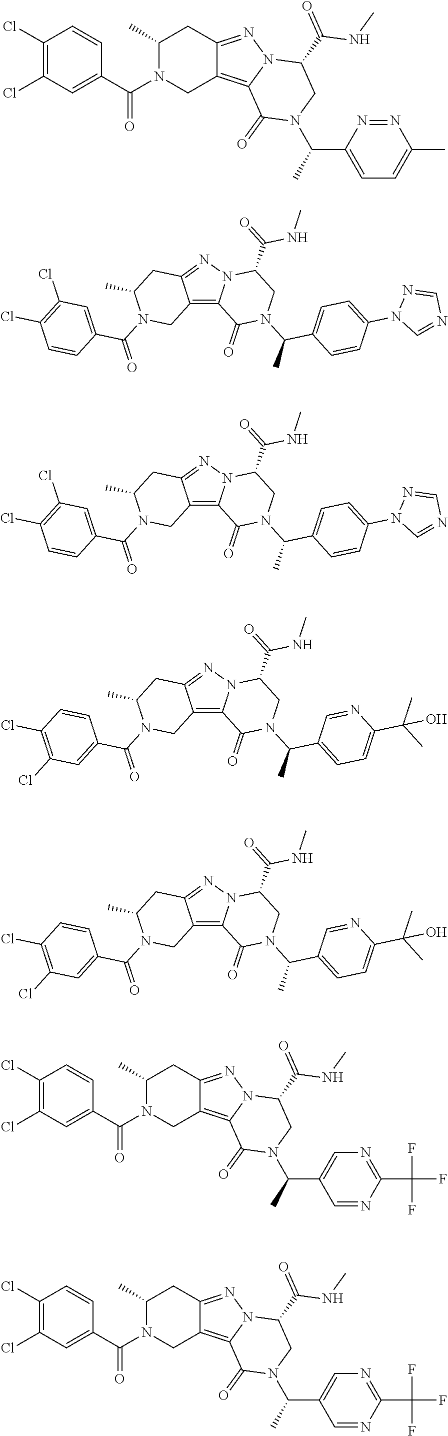 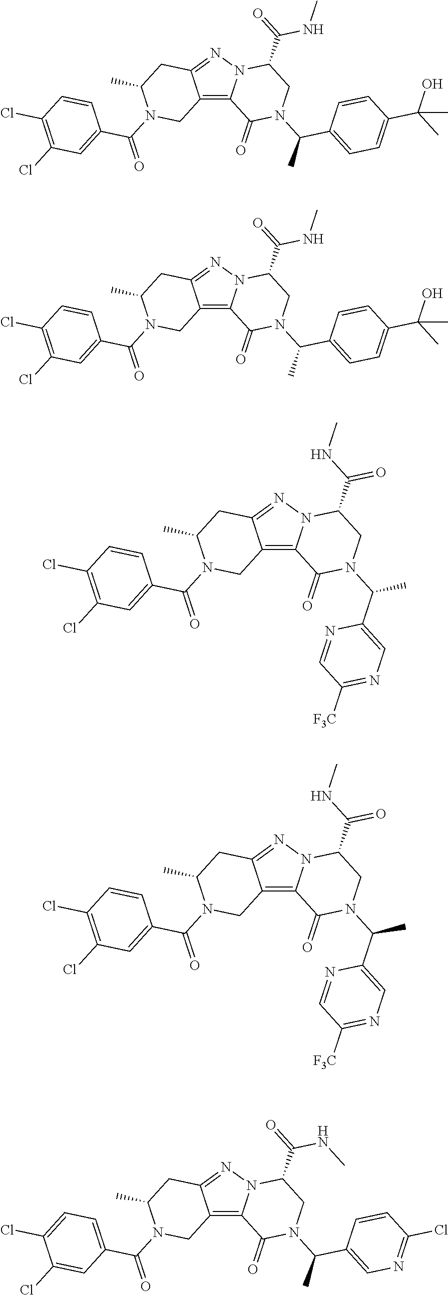 [SMILES: CNC(=O)[C@@H]1CN([C@H](C)c2ccc(C(C)(C)O)cc2)C(=O)c2c3c(nn21)C[C@@H](C)N(C(=O)c1ccc(Cl)c(Cl)c1)C3 ; CNC(=O)[C@@H]1CN([C@@H](C)c2ccc(C(C)(C)O)cc2)C(=O)c2c3c(nn21)C[C@@H](C)N(C(=O)c1ccc(Cl)c(Cl)c1)C3 ; CNC(=O)[C@@H]1CN([C@H](C)c2cnc(C(F)(F)F)cn2)C(=O)c2c3c(nn21)C[C@@H](C)N(C(=O)c1ccc(Cl)c(Cl)c1)C3 ; CNC(=O)[C@@H]1CN([C@@H](C)c2cnc(C(F)(F)F)cn2)C(=O)c2c3c(nn21)C[C@@H](C)N(C(=O)c1ccc(Cl)c(Cl)c1)C3 ; CNC(=O)[C@@H]1CN([C@H](C)c2ccc(Cl)nc2)C(=O)c2c3c(nn21)C[C@@H](C)N(C(=O)c1ccc(Cl)c(Cl)c1)C3] 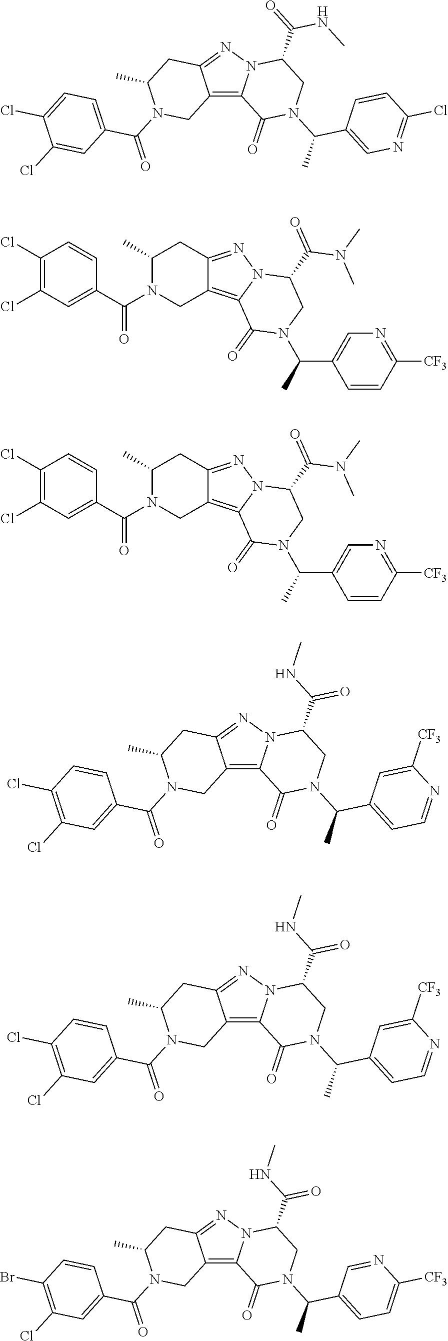 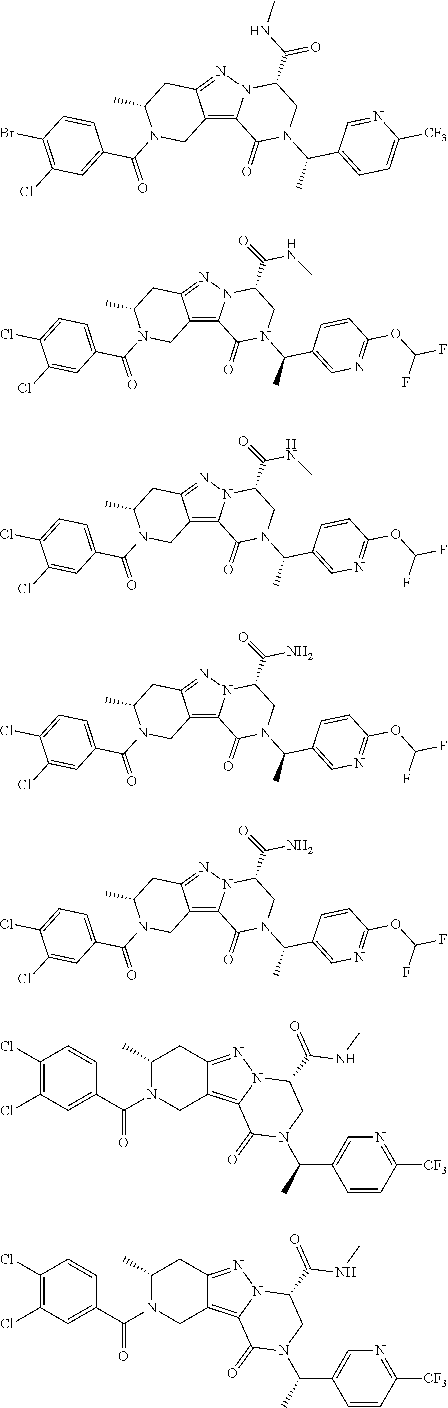 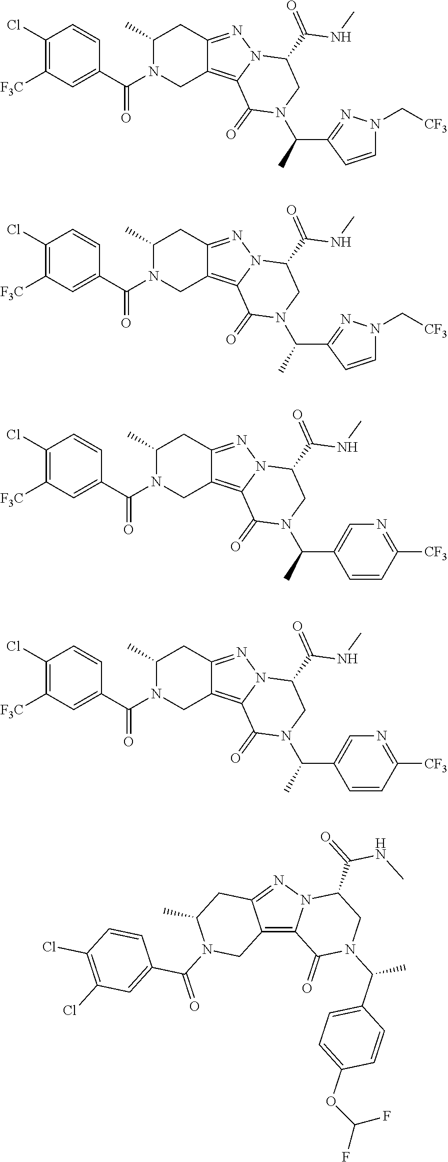 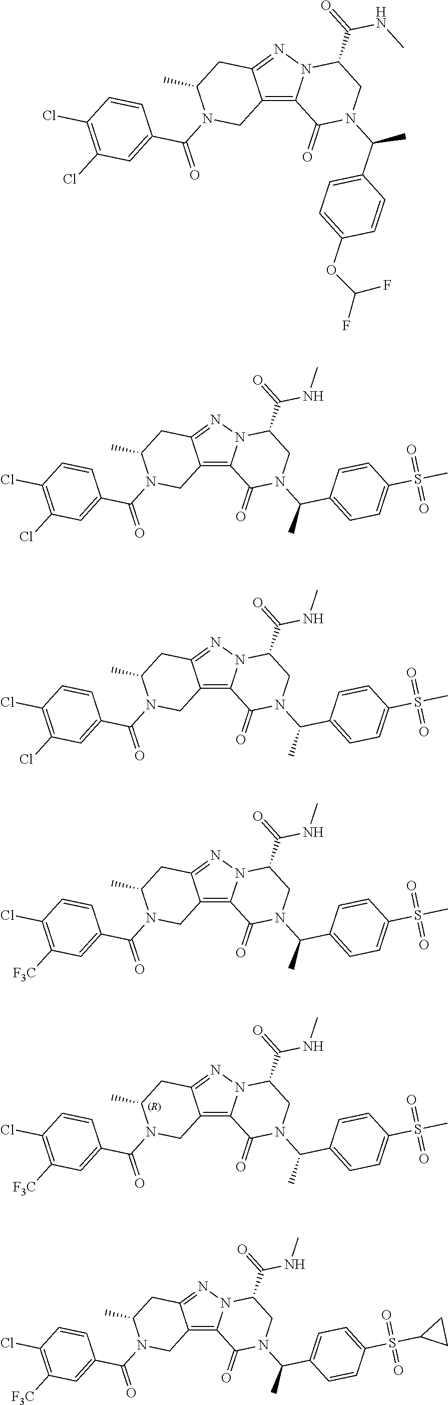 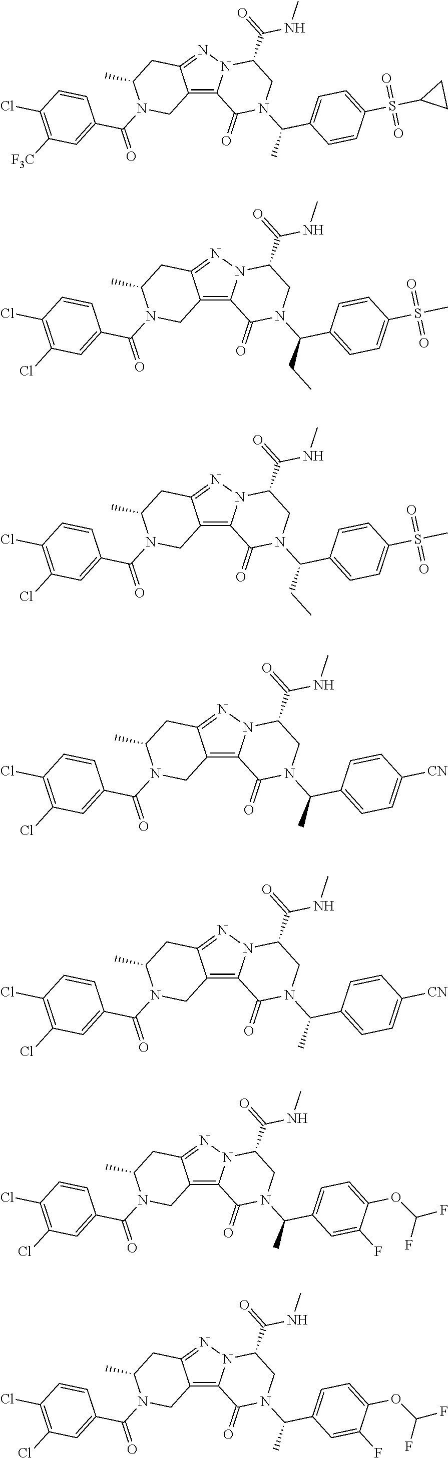 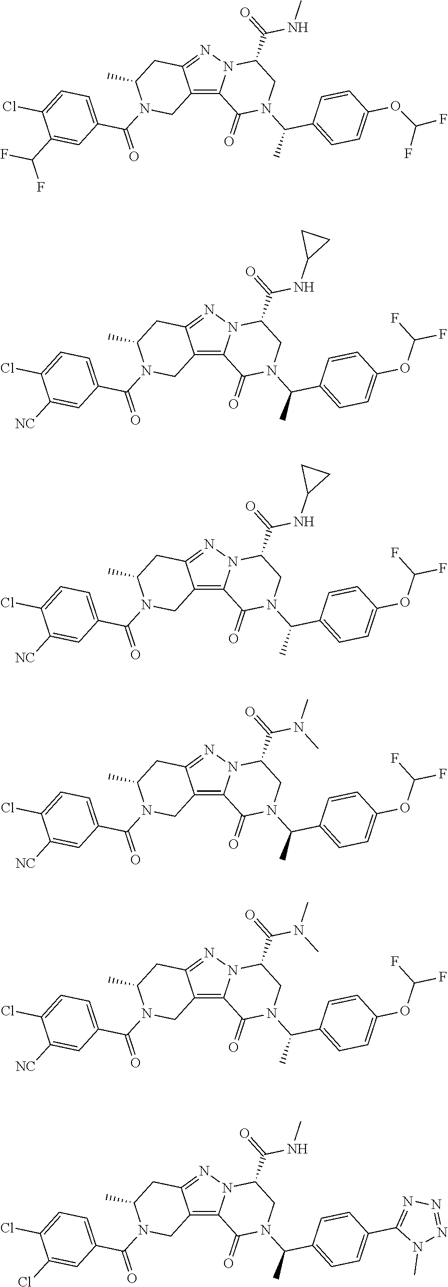 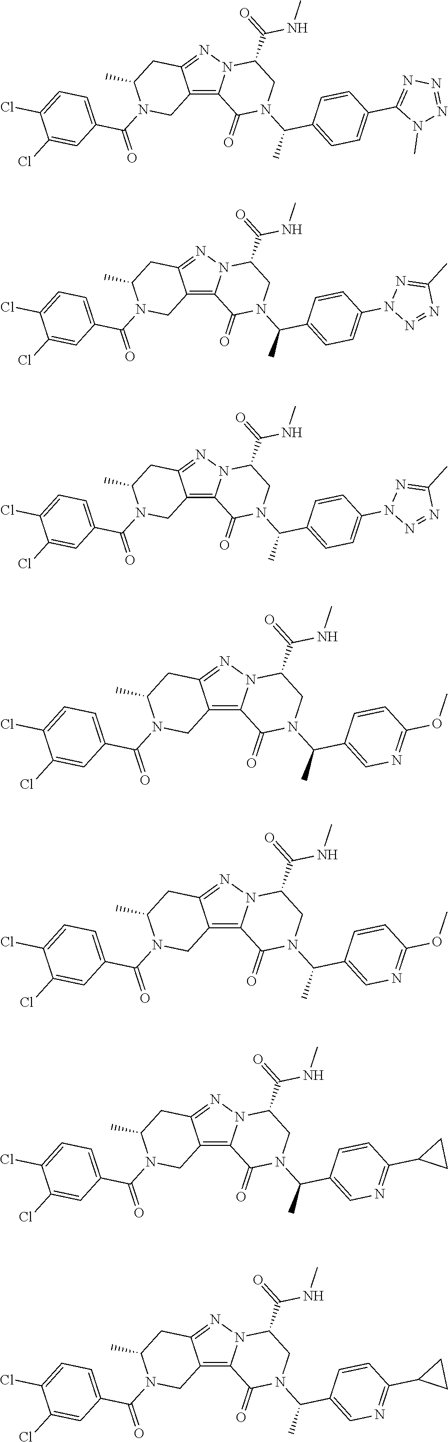 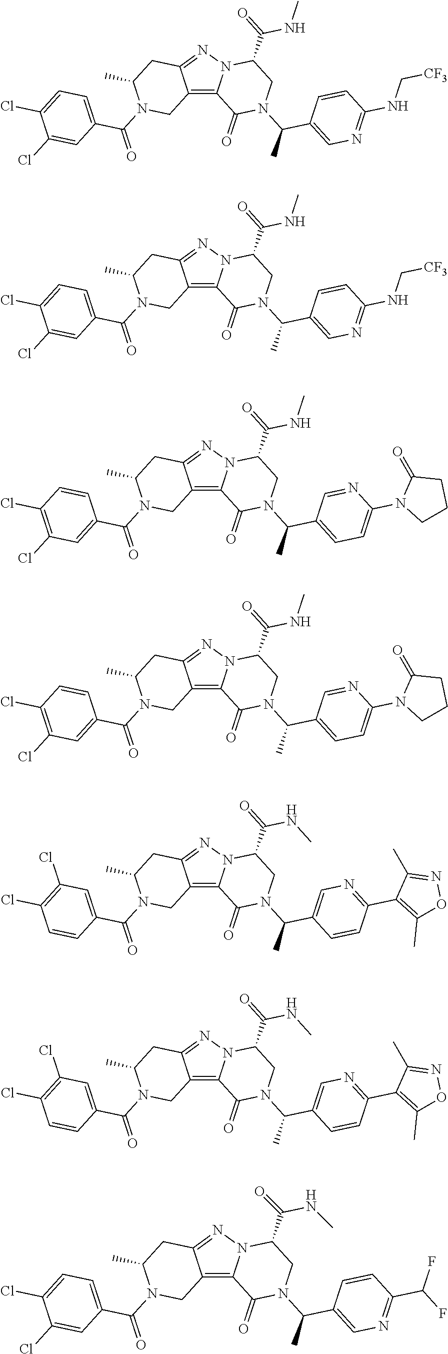 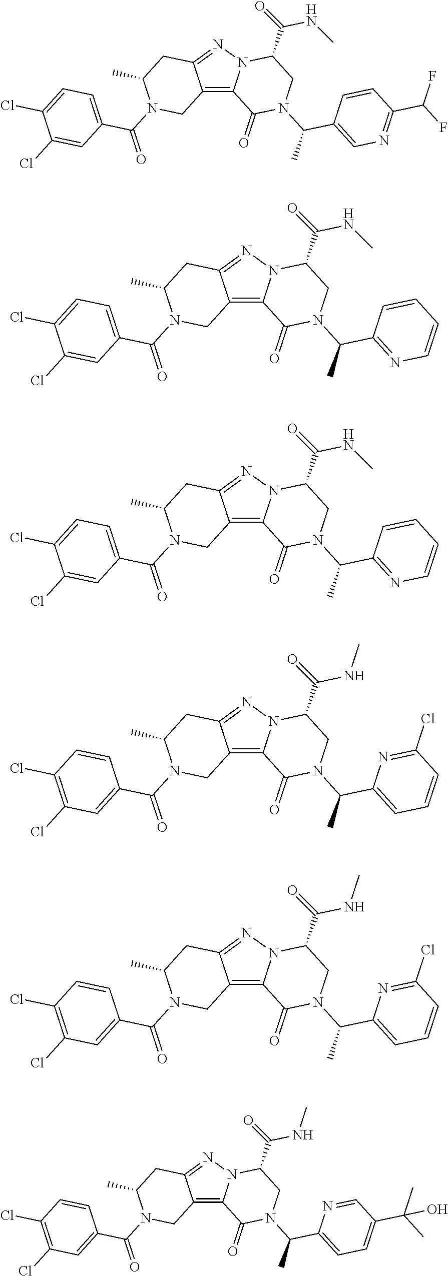 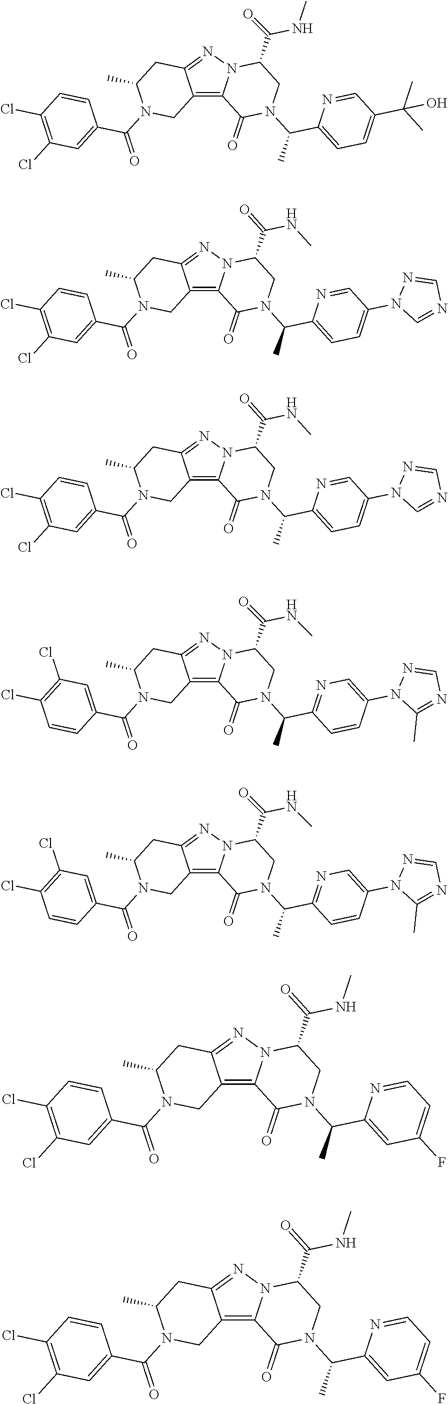 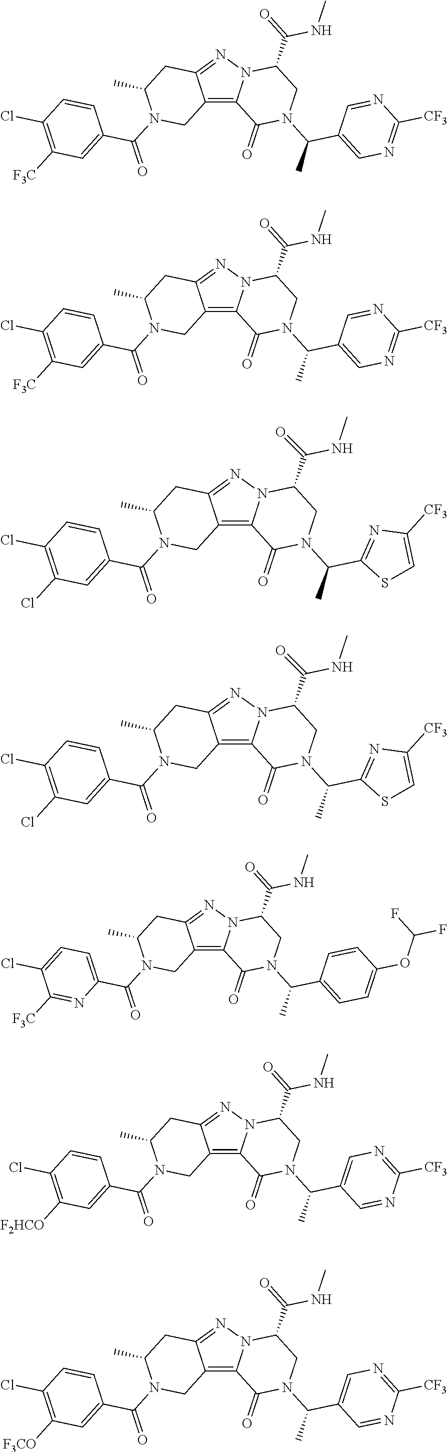
or a pharmaceutically acceptable salt, N-oxide, or a solvate thereof.

18. (canceled)

19. A pharmaceutical composition, which comprises the compound of claim 1, and which further comprises at least one pharmaceutically acceptable excipient.

20-22. (canceled)

23. A method of treating an HBV infection or an HBV-induced disease in an individual in need thereof, comprising administering to the individual a therapeutically effective amount of a compound of Formula (I), 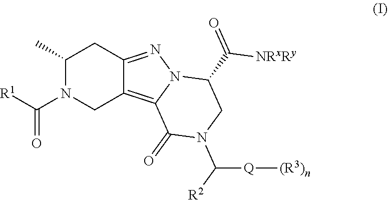
or a stereoisomeric or a tautomeric form thereof, wherein
R¹ is selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₁₋₆alkoxyl, C₃₋₆cycloalkyl and CN, each of C₁₋₆alkyl, C₁₋₆alkoxyl and C₃₋₆cycloalkyl is optionally substituted with 1, 2, 3, 4 or 5 substituents independently selected from the group consisting of halo, hydroxyl and CN;
R² is selected from the group consisting of H, CHF₂, CF₃, C₁₋₆alkyl, C₁₋₆alkylOC₁₋₆alkyl and C₃₋₆Cycloalkyl;
Q represents a ring selected from the group consisting of phenyl, 5-membered heteroaryl and 6-membered heteroaryl;
n represents 0, 1, 2 or 3;
each R³ independently represents a substituent selected from the group consisting of CN, C₁₋₆alkyl, C₁₋₆alkoxyl, C₃₋₆cycloalkyl, 5-membered heteroaryl, 6-membered heteroaryl, 4-8 membered heterocyclyl, halo, N(R_{S})₂, S(O)R_{S} and S(O)₂R_{S}, each of C₁₋₆alkyl, C₁₋₆alkoxyl, C₃₋₆cycloalkyl, 5-membered heteroaryl, 6-membered heteroaryl, 4-8 membered heterocyclyl, N(R_{S})₂, S(O)R_{S} and S(O)₂R_{S} is optionally substituted with 1, 2, 3, 4 or 5 substituents, each of said substituents independently selected from the group consisting of halo, hydroxyl, C₁₋₆alkyl and oxo; R_{S} is each independently selected from the group consisting of H, C₁₋₆alkyl, C₁₋₆alkoxyl and C₃₋₆cycloalkyl;
Rₓ and R_{y} are each independently selected from the group consisting of hydrogen, C₁₋₆alkyl, and C₃₋₆cycloalkyl,
or a pharmaceutically acceptable salt or a solvate thereof;
wherein the following compounds are excluded: 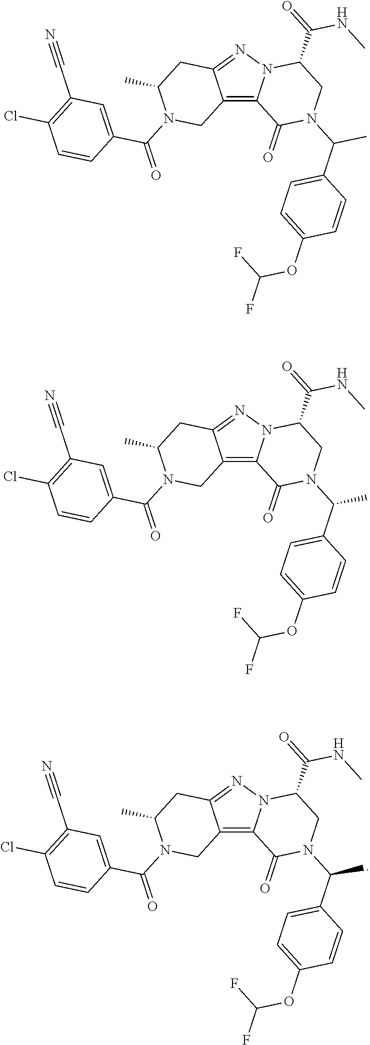 [SMILES: CNC(=O)[C@@H]1CN(C(C)c2ccc(OC(F)F)cc2)C(=O)c2c3c(nn21)C[C@@H](C)N(C(=O)c1ccc(Cl)c(C#N)c1)C3 ; CNC(=O)[C@@H]1CN([C@H](C)c2ccc(OC(F)F)cc2)C(=O)c2c3c(nn21)C[C@@H](C)N(C(=O)c1ccc(Cl)c(C#N)c1)C3 ; CNC(=O)[C@@H]1CN([C@@H](C)c2ccc(OC(F)F)cc2)C(=O)c2c3c(nn21)C[C@@H](C)N(C(=O)c1ccc(Cl)c(C#N)c1)C3]
or a pharmaceutically acceptable salt or a solvate thereof.

24. A product comprising a first compound and a second compound as a combined preparation for simultaneous, separate or sequential use in the prevention or treatment of an HBV infection or of an HBV-induced disease in a subject in need thereof, wherein said first compound is different from said second compound, wherein said first compound is a compound of Formula (I), 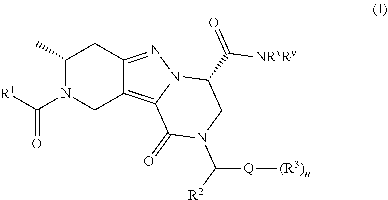
or a stereoisomeric or a tautomeric form thereof, wherein
R¹ is selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₁₋₆alkoxyl, C₃₋₆cycloalkyl and CN, each of C₁₋₆alkyl, C₁₋₆alkoxyl and C₃₋₆cycloalkyl is optionally substituted with 1, 2, 3, 4 or 5 substituents independently selected from the group consisting of halo, hydroxyl and CN;
R² is selected from the group consisting of H, CHF₂, CF₃, C₁₋₆alkyl, C₁₋₆alkylOC₁₋₆alkyl and C₃₋₆Cycloalkyl;
Q represents a ring selected from the group consisting of phenyl, 5-membered heteroaryl and 6-membered heteroaryl;
n represents 0, 1, 2 or 3;
each R³ independently represents a substituent selected from the group consisting of CN, C₁₋₆alkyl, C₁₋₆alkoxyl, C₃₋₆cycloalkyl, 5-membered heteroaryl, 6-membered heteroaryl, 4-8 membered heterocyclyl, halo, N(R_{S})₂, S(O)R_{S} and S(O)₂R_{S}, each of C₁₋₆alkyl, C₁₋₆alkoxyl, C₃₋₆cycloalkyl, 5-membered heteroaryl, 6-membered heteroaryl, 4-8 membered heterocyclyl, N(R_{S})₂, S(O)R_{S} and S(O)₂R_{S} is optionally substituted with 1, 2, 3, 4 or 5 substituents, each of said substituents independently selected from the group consisting of halo, hydroxyl, C₁₋₆alkyl and oxo; R_{S} is each independently selected from the group consisting of H, C₁₋₆alkyl, C₁₋₆alkoxyl and C₃₋₆cycloalkyl;
Rₓ and R_{y} are each independently selected from the group consisting of hydrogen, C₁₋₆alkyl, and C₃₋₆cycloalkyl,
or a pharmaceutically acceptable salt or a solvate thereof;
wherein the following compounds are excluded: 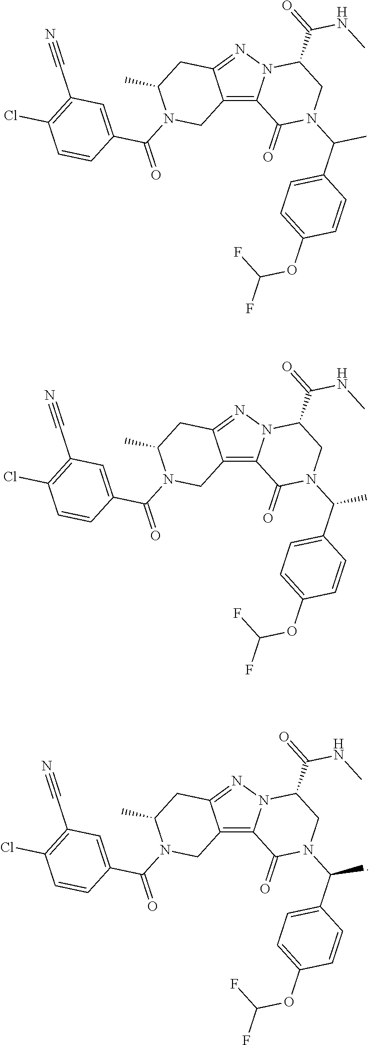 [SMILES: CNC(=O)[C@@H]1CN(C(C)c2ccc(OC(F)F)cc2)C(=O)c2c3c(nn21)C[C@@H](C)N(C(=O)c1ccc(Cl)c(C#N)c1)C3 ; CNC(=O)[C@@H]1CN([C@H](C)c2ccc(OC(F)F)cc2)C(=O)c2c3c(nn21)C[C@@H](C)N(C(=O)c1ccc(Cl)c(C#N)c1)C3 ; CNC(=O)[C@@H]1CN([C@@H](C)c2ccc(OC(F)F)cc2)C(=O)c2c3c(nn21)C[C@@H](C)N(C(=O)c1ccc(Cl)c(C#N)c1)C3]
or a pharmaceutically acceptable salt or a solvate thereof, and wherein said second compound is another HBV inhibitor.

25-26. (canceled)
